(19) United States
(12) Patent Application Publication
(10) Pub. No.: US 2014/0315881 A1
(43) Pub. Date: Oct. 23, 2014

(54) COMPOUNDS AND METHODS

(71) Applicant: Tempero Pharmaceuticals, Inc., Cambridge, MA (US)

(72) Inventors: Erkan Baloglu, Stoneham, MA (US); Gary J. Bohnert, Cambridge, MA (US); Shomir GHOSH, Cambridge, MA (US); Mercedes Lobera, Cambridge, MA (US); Darby R. Schmidt, Arlington, MA (US); Leonard Sung, Cambridge, MA (US)

(21) Appl. No.: 14/235,500

(22) PCT Filed: Jul. 27, 2012
(86) PCT No.: PCT/US12/48735

§ 371 Date: Jan. 28, 2014

Related U.S. Application Data
(60) Provisional application No. 61/513,203, filed on Jul. 29, 2011.
(60) Provisional application No. 61/533,928, filed on Sep. 13, 2011.

(51) Int. Cl. C07D 491/048; C07D 405/04; C07D 413/06; C07D 307/80; C07D 401/04; C07D 417/04; C07D 413/14; C07D 413/04; C07D 405/06

(52) U.S. Cl. CPC C07D 491/048; C07D 413/04; C07D 405/04; C07D 413/06; C07D 405/06; C07D 401/04; C07D 417/04; C07D 413/14; C07D 307/80
USPC 514/210.18; 548/247; 514/378; 546/284.1; 514/337; 544/333; 514/256; 548/217; 514/375; 546/271.7; 514/338; 546/141; 514/309; 546/273.4; 546/167; 514/314; 546/270.1; 544/364; 514/253.11; 514/210.2; 544/353; 514/249; 549/471; 514/469; 540/552; 514/211.09; 540/569; 514/221; 544/137; 514/233.5

(57) ABSTRACT

The present invention relates to novel retinoid-related orphan receptor gamma (RORγ) modulators and their use in the treatment of diseases mediated by RORγ.

The present invention relates to novel retinoid-related orphan receptor gamma (RORγ) modulators and their use in the treatment of diseases mediated by RORγ.

### BACKGROUND OF THE INVENTION

Retinoid-related orphan receptors (RORs) are transcription factors which belong to the steroid hormone nuclear receptor superfamily (Jetten & Joo(2006) Adv. Dev. Biol. 16:313-355). The ROR family consists of three members, ROR alpha (RORα), ROR beta (RORβ), and ROR gamma (RORγ), each encoded by a separate gene (RORA, RORB, and RORC, respectively). RORs contain four principal domains shared by the majority of nuclear receptors: an N-terminal A/B domain, a DNA-binding domain, a hinge domain, and a ligand binding domain. Each ROR gene generates several isoforms which differ only in their N-terminal A/B domain. Two isoforms of RORγ have been identified: RORγ1 and RORγt (also known as RORγ2). RORγ is a term used to describe both RORγ1 and/or RORγt.

While RORγ1 is expressed in a variety of tissues including thymus, muscle, kidney and liver, RORγt is exclusively expressed in the cells of the immune system. RORγt has been identified as a key regulator of Th17 cell differentiation. Th17 cells are a subset of T helper cells which produce IL-17 and other proinflammatory cytokines. Th17 cells have been shown to have key functions in several mouse autoimmune disease models including experimental autoimmune encephalomyelitis (EAE) and collagen-induced arthritis (CIA). In addition, Th17 cells or their products have been shown to be associated with the pathology of a variety of human inflammatory and autoimmune disorders including multiple sclerosis, rheumatoid arthritis, psoriasis, Crohn's disease and asthma (Jetten (2009) Nucl. Recept. Signal. 7:e003; Manel et al. (2008) Nat. Immunol. 9:641-649). The pathogenesis of chronic autoimmune diseases including multiple sclerosis and rheumatoid arthritis arises from the break in tolerance towards self-antigens and the development of auto-aggressive effector T cells infiltrating the target tissues. Studies have shown that Th17 cells are one of the important drivers of the inflammatory process in tissue-specific autoimmunity (Steinman (2008) J. Exp. Med. 205:1517-1522; Leung et al. (2010) Cell. Mol. Immunol. 7:182-189). There is evidence that Th17 cells are activated during the disease process and are responsible for recruiting other inflammatory cells types, especially neutrophils, to mediate pathology in the target tissues (Korn et al. (2009) Annu. Rev. Immunol. 27:485-517).

RORγt plays a critical role in the pathogenic responses of Th17 cells (Ivanov et al. (2006) Cell 126:1121-1133). RORγt deficient mice produce few Th17 cells. In addition, RORγt deficiency resulted in amelioration of EAE. Further support for the role of RORγt in the pathogenesis of autoimmune or inflammatory diseases can be found in the following references: Jetten & Joo (2006) Adv. Dev. Biol. 16:313-355; Meier et al. (2007) Immunity 26:643-654; Aloisi & Pujol-Borrell (2006) Nat. Rev. Immunol. 6:205-217; Jager et al. (2009) J. Immunol. 183:7169-7177; Serafini et al. (2004) Brain Pathol. 14:164-174; Magliozzi et al. (2007) Brain 130:1089-1104; Barnes (2008) Nat. Rev. Immunol. 8:183-192.

In light of the role RORγ plays in the pathogenesis of diseases, it is desirable to prepare compounds that modulate RORγ activity, which can be used in the treatment of diseases mediated by RORγ.

### SUMMARY OF THE INVENTION

The invention is directed to novel RORγ modulators and their use in the treatment of diseases mediated by RORγ. Specifically, the invention is directed to compounds according to Formula (I):

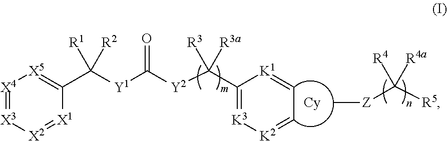

wherein:

m and n are each independently 0, 1, or 2;

X¹, X², X³, X⁴, and X⁵ are each independently selected from N, N⁺—O⁻, CH, and CR⁶, wherein 0-3 of X¹, X², X³, X⁴, and X⁵ are N or N⁺—O⁻ and 0-3 of X¹, X², X³, X⁴, and X⁵ are CR⁶;

one of Y¹ and Y² is O or NR⁸ and the other is a bond;

or X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;

K¹, K², and K³ are each independently selected from N, CH, and CR⁶, wherein 0-2 of K¹, K², and K³ are N and 0-2 of K¹, K², and K³ are CR⁶;

Z is O, C═O, NH, or a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

R¹¹ is (C₁-C₆)alkyl, or (C₁-C₆)haloalkyl; and

Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R¹⁰;

or a salt thereof, particularly, a pharmaceutically acceptable salt thereof.

In another aspect, this invention provides a pharmaceutical composition comprising a compound of Formula (I), or a pharmaceutically acceptable salt thereof, and a pharmaceutically acceptable excipient.

In another aspect, this invention provides for the use of a compound of Formula (I) or a pharmaceutically acceptable salt thereof for the treatment of diseases mediated by RORγ. The invention further provides for the use of a compound of Formula (I) or a pharmaceutically acceptable salt thereof as an active therapeutic substance in the treatment of a disease mediated by RORγ.

In another aspect, the invention provides a compound of Formula (I) or a pharmaceutically acceptable salt thereof for use in therapy.

In another aspect, the invention provides the use of a compound of Formula (I) or a pharmaceutically acceptable salt thereof in the manufacture of a medicament for the treatment of diseases mediated by RORγ.

Examples of such diseases for which compounds of Formula (I) may be used include autoimmune or inflammatory diseases such as multiple sclerosis, rheumatoid arthritis, psoriasis, uveitis, dry eye, glomerulonephritis, Crohn's disease and asthma, especially psoriasis

In yet another aspect, the invention is directed to methods of treating such diseases for example by administering to a patient (e.g. human) in need thereof an effective amount of a compound of Formula (I) or a pharmaceutically acceptable salt thereof.

### DETAILED DESCRIPTION OF THE INVENTION

As used herein, the term “alkyl” represents a saturated, straight, or branched hydrocarbon moiety. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl. C₀alkyl means that no alkyl group is present in the moiety. Thus, —((C₀)alkyl)CONH₂ is equivalent to —CONH₂.

When the term “alkyl” is used in combination with other substituent groups, such as “haloalkyl”, “hydroxyalkyl”, “alkoxyalkyl”, “arylalkyl”, or “heteroarylalkyl”, the term “alkyl” is intended to encompass a divalent straight or branched-chain hydrocarbon radical. For example, “arylalkyl” is intended to mean the radical-alkylaryl, wherein the alkyl moiety thereof is a divalent straight or branched-chain carbon radical and the aryl moiety thereof is as defined herein, and is represented by, for example, the bonding arrangement present in a benzyl group (—CH₂-phenyl); “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical, and is represented by, for example, a trifluoromethyl group (—CF₃).

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” means an alkyl radical containing the specified number of carbon atoms attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Aryl” represents a group or moiety comprising an aromatic, monovalent monocyclic or bicyclic hydrocarbon radical containing from 6 to 10 carbon ring atoms, which may be fused to one or more cycloalkyl rings.

Generally, in the compounds of this invention, aryl is phenyl.

Heterocyclic groups may be heteroaryl or heterocycloalkyl groups.

“Heteroaryl” represents a group or moiety comprising an aromatic monovalent monocyclic or bicyclic radical, containing 5 to 10 ring atoms, including 1 to 4 heteroatoms independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including 1 to 4 heteroatoms independently selected from nitrogen, oxygen and sulfur. Illustrative examples of heteroaryls useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Generally, the heteroaryl groups present in the compounds of this invention are 5-membered and/or 6-memebred monocyclic heteroaryl groups. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur. Illustrative examples of heterocycloalkyls useful in the present invention include, but are not limited to, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-7 membered heterocycloalkyl groups, such as pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, and hexahydro-1H-1,4-diazepinyl.

It is to be understood that the terms heterocyclic, heteroaryl, and heterocycloalkyl are intended to encompass stable heterocyclic, heteroaryl, or heterocycloalkyl groups where a ring nitrogen heteroatom is optionally oxidized (e.g., heteroaryl groups containing an N-oxide, such as pyridinyl-N-oxide) or where a ring sulfur heteroatom is optionally oxidized (e.g., heterocycloalkyl groups containing sulfones or sulfoxide moieties, such as tetrahydrothienyl-1-oxide (a tetrahydrothienyl sulfoxide) or tetrahydrothienyl-1,1-dioxide (a tetrahydrothienyl sulfone)).

“Oxo” represents a double-bonded oxygen moiety; for example, if attached directly to a carbon atom forms a carbonyl moiety (C═O).

Further, it is to be understood that when a heteroaryl (e.g. pyridyl) is substituted by hydroxyl, the resulting hydroxy-substitued heteroaryl group may exist predominantly in a tautomeric form where the hydroxyl appears as an oxo substituent on a corresponding partially unsaturated heterocycloalkyl (e.g., a 2-oxo-1,2-dihydropyridinyl group). For the purposes of this invention, a hydroxy substituent on a heteroaryl group, particularly pyridyl, is intended to encompass such an oxo substituted, partially unsaturated heterocycloalkyl group, particularly 2-oxo-1,2-dihydropyridinyl.

The terms “halogen” and “halo” represent chloro, fluoro, bromo, or iodo substituents. “Hydroxy” or “hydroxyl” is intended to mean the radical —OH.

“RORγ” refers to all isoforms encoded by the RORC gene which include RORγ1 and RORγt.

“RORγ modulator” refers to a chemical compound that inhibits, either directly or indirectly, the activity of RORγ. RORγ modulators include antagonists and inverse agonists of RORγ.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, the term “compound(s) of the invention” means a compound of Formula (I) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” indicates that a group, such as alkyl, cycloalkyl, alkoxy, heterocycloalkyl, aryl, or heteroaryl, may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different. The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions.

This invention is further directed to the compounds according to Formula (I), wherein:

m and n are each independently 0, 1, or 2;

X¹, X², X³, X⁴, and X⁵ are each independently selected from N, N⁺—O⁻, CH, and CR⁶, wherein 0-3 of X¹, X², X³, X⁴, and X⁵ are N or N⁺—O⁻ and 0-3 of X¹, X², X³, X⁴, and X⁵ are CR⁶;

one of Y¹ and Y² is O or NR⁸ and the other is a bond;

or X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;

K¹, K², and K³ are each independently selected from N, CH, and CR⁶, wherein 0-2 of K¹, K², and K³ are N and 0-2 of K¹, K², and K³ are CR⁶;

Z is O, C═O, NH, or a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R¹⁰;

or a salt thereof, particularly, a pharmaceutically acceptable salt thereof.

Suitably, m and n are each independently 0, 1, or 2. In another embodiment of this invention, m is 1 and n is 0 or 1.

Suitably, X¹, X², X³, X⁴, and X⁵ are each independently selected from N, N⁺—O⁻ (i.e. N-oxide), CH, and CR⁶, wherein 0-3 of X¹, X², X³, X⁴, and X⁵ are N or N⁺—O⁻ and 0-3 of X¹, X², X³, X⁴, and X⁵ are CR⁶. In another embodiment of this invention, X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶. In another embodiment of this invention, X¹ and X⁵ are each independently selected from N, N⁺—O⁻, CH, and CR⁶, and X², X³, and X⁴ are each independently selected from CH and CR⁶, wherein at least one of X¹ and X⁵ is N or N⁺—O⁻ and 0-3 of X¹, X², X³, X⁴, and X⁵ are CR⁶. In another embodiment of this invention, X¹ and X⁵ are each independently selected from N, N⁺—O⁻, and a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. N, N⁺—O⁻, CH, and CR⁶, wherein R⁶ is halogen, cyano, (C₁-C₄)alkyl, (C₁-C₄)haloalkyl, (C₁-C₄)alkoxy, or ((C₁-C₄)alkyl)((C₁-C₄)alkyl)amino), and X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. CH or CR⁶, wherein R⁶ is halogen, cyano, (C₁-C₄)alkyl, (C₁-C₄)haloalkyl, (C₁-C₄)alkoxy, or ((C₁-C₄)alkyl((C₁-C₄)alkyl)amino), wherein at least one of X¹ and X⁵ is N or N⁺—O⁻ and 2-4 of X¹, X², X³, X⁴, and X⁵ are a carbon atom substituted by hydrogen (i.e. CH). In another embodiment of this invention, X² is N or N⁺—O⁻, and X¹, X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X³, X⁴, and X⁵ are a carbon atom substituted by hydrogen. In another embodiment of this invention, X¹, X², X³, X⁴, and X⁵ are each independently selected from CH and CR⁶, wherein 0-3 of X¹, X², X³, X⁴, and X⁵ are CR⁶. In another embodiment of this invention, X¹, X², X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-5 of X¹, X², X³, X⁴, and X⁵ are a carbon atom substituted by hydrogen. In another embodiment of this invention, X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

Suitably, one of Y¹ and Y² is O or NR⁸ and the other is a bond. In another embodiment of this invention, one of Y¹ and Y² is O, NH, or N((C₁-C₄)alkyl) and the other is a bond. In a specific embodiment of this invention, Y¹ is NH or NCH₃ and Y² is a bond. In another specific embodiment of this invention, Y¹ is NH and Y² is a bond. In another specific embodiment of this invention, Y¹ is a bond and Y² is NH.

In another embodiment of this invention, X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl. In another embodiment of this invention, X¹ is CR⁶, Y¹ is NR⁸, Y² is a bond, and R⁶ and R⁸ taken together represent —CH₂—, —CH₂CH₂—, or —CH₂CH₂CH₂—.

Suitably, K¹, K², and K³ are each independently selected from N, CH, and CR⁶, wherein 0-2 of K¹, K², and K³ are N and 0-2 of K¹, K², and K³ are CR⁶. In another embodiment of this invention, K¹, K², and K³ are each independently selected from CH and CR⁶, wherein 0-2 of K¹, K², and K³ are CR⁶. In another embodiment of this invention, K¹, K², and K³ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. CH or CR⁶, wherein R⁶ is halogen, (C₁-C₄)alkyl, (C₁-C₄)haloalkyl, cyano, (C₁-C₄)alkoxy, or ((C₁-C₄)alkyl)((C₁-C₄)alkyl)amino), wherein 1-3 of K¹, K², and K³ are a carbon atom substituted by hydrogen (i.e. CH). In a specific embodiment of this invention, K¹, K², and K³ are each independently CH.

Suitably, Z is O, C═O, NH, or a bond. In another embodiment of this invention, Z is O, C═O, or a bond. In a specific embodiment of this invention, Z is a bond.

Suitably, R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶. In another embodiment of this invention, R¹ is (C₃-C₆)alkyl, (C₃-C₈)cycloalkyl, (C₁-C₆)alkoxy(C₁-C₂)alkyl, aryl, or heteroaryl, each of which is optionally substituted one, two, or three times, independently, by R⁶. In another embodiment of this invention, R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. wherein R⁶ is halogen, (C₁-C₄)alkyl, (C₁-C₄)haloalkyl, cyano, (C₁-C₄)alkoxy, or ((C₁-C₄)alkyl)((C₁-C₄)alkyl)amino). In another embodiment of this invention, R¹ is (C₃-C₆)alkyl. In another embodiment of this invention, R¹ is (C₅-C₆)alkyl. In another embodiment of this invention, R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl((C₁-C₄)alkyl)amino. In a specific embodiment of this invention, R¹ is phenyl or pyridinyl. In another specific embodiment of this invention, R¹ is phenyl.

Suitably, R² is hydrogen, (C₁-C₄)alkyl, or (C₁-C₄)haloalkyl. In another embodiment of this invention, R² is hydrogen or (C₁-C₄)alkyl. In another embodiment of this invention, R² is hydrogen or methyl. In a specific embodiment of this invention, R² is hydrogen.

In another embodiment of this invention, R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶. In another embodiment of this invention, R¹ and R² taken together represent —CH₂CH₂CH₂—, —CH₂CH₂CH₂CH₂—, or —CH₂CH₂CH₂CH₂CH₂—.

Suitably, R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino. In another embodiment of this invention, R³ and R^{3a} are each independently hydrogen or methyl. In a specific embodiment of this invention, R³ and R^{3a} are each independently hydrogen.

Suitably, R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl. In another embodiment of this invention, R⁴ is (C₁-C₄)alkyl, (C₁-C₄)haloalkyl, —OR⁹, or —NR⁸R⁹, wherein said (C₁-C₄)alkyl or (C₁-C₆)haloalkyl is optionally substituted by hydroxyl, —OR⁹, —CO₂R⁷, —CONR⁷R⁸, or —NR⁸R⁹. In another embodiment of this invention, R⁴ is halogen, (C₁-C₄)alkyl, (C₁-C₄)alkylamino, ((C₁-C₄)alkyl)((C₁-C₄)alkyl)amino, (C₁-C₄)alkoxy(C₁-C₄)alkylamino, —SO₂(C₁-C₄)alkyl, —NHCO₂(C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₄)alkyl)CO₂R⁷, —O((C₁-C₄)alkyl)CONH₂, —O((C₁-C₄)alkyl)CONH(C₁-C₄)alkyl, —O((C₁-C₄)alkyl)CON((C₁-C₄)alkyl)((C₁-C₄)alkyl), —SO₂(C₁-C₄)alkyl, or CO₂R⁷. In another embodiment of this invention, R⁴ is halogen, (C₁-C₄)alkyl, (C₁-C₄)alkylamino, ((C₁-C₄)alkyl((C₁-C₄)alkyl)amino, (C₁-C₄)alkoxy(C₁-C₄)alkylamino, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, or —O((C₁-C₃)alkyl)CON((C₁-C₄)alkyl)((C₁-C₄)alkyl). In another embodiment of this invention, R⁴ is (C₁-C₄)alkoxy, hydroxy(C₂-C₄)alkoxy, (C₁-C₄)alkoxy(C₂-C₄)alkoxy, amino(C₂-C₄)alkoxy, —SO₂(C₁-C₄)alkyl, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, or —O((C₁-C₃)alkyl)CON((C₁-C₄)alkyl((C₁-C₄)alkyl). In another embodiment of this invention, R⁴ is (C₁-C₄)alkoxy, —SO₂(C₁-C₄)alkyl, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, or —O((C₁-C₃)alkyl)CON((C₁-C₄)alkyl)((C₁-C₄)alkyl).

In another embodiment of this invention, R^{4a} is hydrogen, halogen, or (C₁-C₄)alkyl. In another embodiment of this invention, R^{4a} is hydrogen, fluorine, or methyl. In another embodiment of this invention, R^{4a} is hydrogen or methyl. In a specific embodiment of this invention, R^{4a} is hydrogen. In another specific embodiment of this invention, R^{4a} is methyl.

In another embodiment of this invention, R⁴ and R^{4a} are each hydrogen. In another embodiment of this invention, R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷. In another embodiment of this invention, R⁴ and R^{4a} taken together represent —CH₂CH₂—, —CH₂CH₂CH₂—, —CH₂CH₂CH₂CH₂—, or —CH₂CH₂CH₂CH₂CH₂—.

Suitably, R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, R⁵ is 5- or 6-membered heteroaryl which is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, R⁵ is 5- or 6-membered heteroaryl which is optionally substituted one, two, or three times, independently, by (C₁-C₄)alkyl, (C₁-C₄)haloalkyl, (C₃-C₆)cycloalkyl, halogen, cyano, hydroxyl, hydroxy(C₁-C₆)alkyl, (C₁-C₄)alkoxy, —((C₀-C₃)alkyl)CO₂H, ((C₀-C₃)alkyl)CO₂(C₁-C₄)alkyl, —((C₀-C₃)alkyl)CONH₂, —((C₀-C₃)alkyl)CONH(C₁-C₄)alkyl, —((C₀-C₃)alkyl)CON((C₁-C₄)alkyl)((C₁-C₄)alkyl), (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, or ((C₁-C₄)alkyl)((C₁-C₄)alkyl)amino. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl. In another embodiment of this invention, R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, or isothiazolyl, each of which is optionally substituted one or two times, independently, by (C₁-C₄)alkyl. In another embodiment of this invention, R⁵ is isoxazolyl which is optionally substituted one or two times, independently, by (C₁-C₄)alkyl. In another embodiment of this invention, R⁵ is pyridinyl, pyridinyl N-oxide, pyridazinyl, pyrazinyl, pyrimidinyl, or triazinyl, each of which is optionally substituted one or two times, independently, by (C₁-C₄)alkyl. In another embodiment of this invention, R⁵ is a pyridinyl or pyridinyl N-oxide group, which group is optionally substituted one or two times, independently, by (C₁-C₄)alkyl.

Suitably, Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R¹⁰. In another embodiment of this invention, Cy taken together with the two carbon atoms of the phenyl or heteroaryl group to which it is fused comprises a five membered aromatic ring, containing a heteroatom selected from oxygen, nitrogen, and sulfur and optionally containing an additional nitrogen atom, which ring is optionally substituted by (C₁-C₄)alkyl. In another embodiment of this invention, Cy taken together with the two carbon atoms of the phenyl or heteroaryl group to which it is fused comprises a six membered aromatic ring, optionally containing one or two nitrogen atoms, which ring is optionally substituted by (C₁-C₄)alkyl, (C₁-C₄)haloalkyl, (C₁-C₄)alkoxy, halogen, or hydroxyl.

One particular embodiment of the invention is a compound of Formula (Ia):

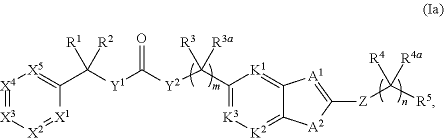

wherein:

m is 1;

n is 0 or 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹ is N, CH, or CR¹⁰;

A² is O, S, NH, or NR⁹;

Z is a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

R¹¹ is (C₁-C₆)alkyl, or (C₁-C₆)haloalkyl;

or a pharmaceutically acceptable salt thereof.

Another particular embodiment of the invention is a compound of Formula (Ib):

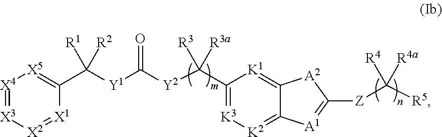

wherein:

m is 1;

n is 0 or 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹ is N, CH, or Cie;

A² is O, S, NH, or NR⁹;

Z is a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —OC₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

R¹¹ is (C₁-C₆)alkyl, or (C₁-C₆)haloalkyl;

or a pharmaceutically acceptable salt thereof.

Another particular embodiment of the invention is a compound of Formula (Ia) or (Ib) wherein:

m is 1;

n is 0 or 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹ is N or CH;

A² is O, S, NH, or N((C₁-C₄)alkyl;

Z is a bond;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydrogen, (C₁-C₄)alkyl, (C₁-C₄)haloalkyl, —SO₂R¹¹, —OR⁹, or —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;

R^{4a} is hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷; and

R¹¹ is (C₁-C₆)alkyl, or (C₁-C₆)haloalkyl;

or a pharmaceutically acceptable salt thereof.

Another particular embodiment of the invention is a compound of Formula (Ia) or (Ib), wherein:

m is 1; n is 0 or 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)allyl)((C₁-C₄)allyl)amino, wherein 2-4 of X², X³, X⁴, and X⁵ are a carbon atom substituted by hydrogen;

Y¹ is NH and Y² is a bond;

K¹, K², and K³ are each independently CH;

A¹ is N or CH;

A² is O, S, NH, or N((C₁-C₄)alkyl;

Z is a bond;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)allyl)amino;

R² is hydrogen;

R³ and R^{3a} are each independently hydrogen or methyl;

R⁴ is (C₁-C₄)alkoxy, hydroxy(C₂-C₄)alkoxy, (C₁-C₄)alkoxy(C₂-C₄)alkoxy, amino(C₂-C₄)alkoxy, —SO₂(C₁-C₄)alkyl, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, or —O((C₁-C₃)alkyl)CON((C₁-C₄)alkyl)((C₁-C₄)allyl);

R^{4a} is hydrogen; and

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl;

or a pharmaceutically acceptable salt thereof.

Another particular embodiment of the invention is a compound of Formula (Ic):

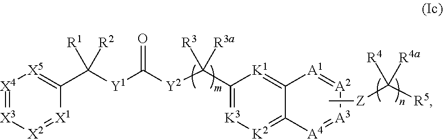

wherein:

m is 1;

n is 0 or 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹, A², A³, and A⁴ are each independently selected from N, C, CH, and CR¹⁰, wherein 0-2 of A¹, A², A³, and A⁴ are N, 0-1 of A¹, A², A³, and A⁴ are CR¹⁰, and 1 of A¹, A², A³, and A⁴ is C to which Z is attached,

Z is a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

R¹¹ is (C₁-C₆)alkyl, or (C₁-C₆)haloalkyl;

or a pharmaceutically acceptable salt thereof.

Another particular embodiment of the invention is a compound of Formula (Ic) wherein:

m is 1;

n is 0 or 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹, A², A³, and A⁴ are each independently selected from N, C, and CH, wherein 1-2 of A¹, A², A³, and A⁴ are N, and 1 of A¹, A², A³, and A⁴ is C to which Z is attached;

R¹ is phenyl optionally substituted one or two times, independently, by halogen, (C₁-C₄)alkyl, (C₁-C₄)halo alkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydrogen, (C₁-C₄)alkyl, (C₁-C₄)haloalkyl, —SO₂R¹¹, —OR⁹, or —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;

R^{4a} is hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl;

R⁷ is hydrogen, (C₁-C₆)alkyl, (C₁-C₆)halo alkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)halo alkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl) ((C₁-C₄)alkyl), amino, (C₁-C₄)alkylamino, ((C₃-C₁)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷; and

R¹¹ is ((C₁-C₆)alkyl, or (C₁-C₆)haloalkyl;

or a pharmaceutically acceptable salt thereof.

Another particular embodiment of the invention is a compound of Formula (Id):

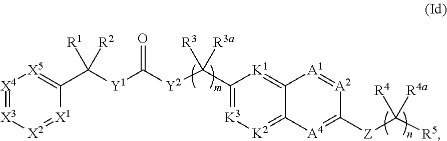

wherein:

m is 1;

n is 0 or 1;

X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;

Y¹ is NH or NCH₃ and Y² is a bond;

K¹, K², and K³ are each independently selected from N, CH, and CR⁶, wherein 0-1 of K¹, K², and K³ are N and 0-1 of K¹, K², and K³ are CR⁶;

A¹, A², and A⁴ are each independently selected from N, CH, and CR¹⁰, wherein 0-2 of A¹, A², and A⁴ are N, and 0-1 of A¹, A², and A⁴ are CR¹⁰;

Z is a bond;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;

or R⁴ and R^{4a} are each hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and

R¹¹ is (C₁-C₆)alkyl, or (C₁-C₆)haloalkyl;

or a pharmaceutically acceptable salt thereof.

Another particular embodiment of the invention is a compound of Formula (Id) wherein:

m is 1;

n is 0 or 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen;

Y¹ is NH and Y² is a bond;

K¹, K², and K³ are each independently CH;

A¹, A², and A⁴ are each independently selected from N and CH, wherein 1-2 of A¹, A², and A⁴ are N;

Z is a bond;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

R⁴ is hydrogen, (C₁-C₄)alkyl, (C₁-C₄)haloalkyl, —SO₂R¹¹, —OR⁹, or —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;

R^{4a} is hydrogen;

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷; and

R¹¹ is (C₁-C₆)alkyl, or (C₁-C₆)haloalkyl;

or a pharmaceutically acceptable salt thereof.

Another particular embodiment of the invention is a compound of Formula (Id) wherein:

m is 1;

n is 0 or 1;

X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)allyl)((C₁-C₄)allyl)amino, wherein 2-4 of X², X³, X⁴, and X⁵ are a carbon atom substituted by hydrogen;

Y¹ is NH and Y² is a bond;

K¹, K², and K³ are each independently CH;

A¹, A², and A⁴ are each independently selected from N and CH, wherein 1-2 of A¹, A², and A⁴ are N;

Z is a bond;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)allyl)amino;

R² is hydrogen;

R³ and R^{3a} are each independently hydrogen or methyl;

R⁴ is (C₁-C₄)alkoxy, hydroxy(C₂-C₄)alkoxy, (C₁-C₄)alkoxy(C₂-C₄)alkoxy, amino(C₂-C₄)alkoxy, —SO₂(C₁-C₄)alkyl, —O((C₁-C₃)alkyl)CO₂H, —O((C₁-C₃)alkyl)CO₂(C₁-C₄)alkyl, —O((C₁-C₃)alkyl)CON H₂, —O((C₁-C₃)allyl)CONH(C₁-C₄)alkyl, or —O((C₁-C₃)alkyl)CON((C₁-C₄)alkyl((C₁-C₄)alkyl;

R^{4a} is hydrogen; and

R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by (C₁-C₄)alkyl;

or a pharmaceutically acceptable salt thereof.

Specific compounds of this invention include:
N-((4-chlorophenyl)(phenyl)methyl)-2-(2-(3,5-dimethylisoxazol-4-yl)benzofuran-5-yl)acetamide;
2-(2-(3,5-dimethylisoxazol-4-yl)benzofuran-5-yl)-N-(1-(2,4-dimethylphenyl)-4-methylpentyl)acetamide;
N-((2,4-dimethylphenyl)(phenyl)methyl)-2-(2-(pyridin-3-yl)benzofuran-5-yl)acetamide;
2-(2-(3,5-dimethylisoxazol-4-yl)benzofuran-5-yl)-N-((2,4-dimethylphenyl)(phenyl)methyl)-2-methylpropanamide;
2-(2-(3,5-dimethylisoxazol-4-yl)benzofuran-5-yl)-N-(phenyl(p-tolyl)methyl)acetamide;
N-((2,4-dichlorophenyl)(phenyl)methyl)-2-(2-(3,5-dimethylisoxazol-4-yl)benzofuran-5-yl)acetamide;
N-((4-chlorophenyl)(phenyl)methyl)-2-(2-(3,5-dimethylisoxazol-4-yl)benzofuran-5-yl)-2-methylpropanamide;
N-((4-chloro-2-methylphenyl)(phenyl)methyl)-2-(2-(3,5-dimethylisoxazol-4-yl)benzofuran-5-yl)-2-methylpropanamide;
2-(2-(3,5-dimethylisoxazole-4-carbonyl)benzofuran-5-yl)-N-((2,4-dimethylphenyl)(phenyl)methyl)acetamide;
N-((4-chloro-2-methylphenyl)(phenyl)methyl)-2-(2-(pyrimidin-5-yl)benzofuran-5-yl)acetamide;
methyl 3-(5-(2-(((2,4-dimethylphenyl)(phenyl)methyl)amino)-2-oxoethyl)benzofuran-2-yl)picolinate;
N-((4-chloro-2-methylphenyl)(phenyl)methyl)-2-(2-(2-(hydroxymethyl)pyridin-3-yl)benzofuran-5-yl)acetamide;
2-((2-((3,5-dimethylisoxazol-4-yl)(ethoxy)methyl)benzofuran-5-yl)-N-((2,4-dimethylphenyl)(phenyl)methyl)acetamide;
2-(2-((3,5-dimethylisoxazol-4-yl)methyl)benzofuran-5-yl)-N-((2,4-dimethylphenyl)(phenyl)methyl)acetamide;
N-((4-chloro-2-methylphenyl)(phenyl)methyl)-2-(2-((2-methylpyridin-3-yl)methyl)benzofuran-5-yl)acetamide;
N-((2,4-dimethylphenyl)(phenyl)methyl)-2-(2-(2-methylpyridin-3-yl)methyl)benzofuran-5-yl)acetamide;
2-(2-(3,5-dimethylisoxazol-4-yl)benzo[d]oxazol-5-yl)-N-((2,4-dimethylphenyl)(phenyl)methyl)acetamide;
N-((4-chloro-2-methylphenyl)(phenyl)methyl)-2-(2-(2,4-dimethylpyridin-3-yl)benzo[d]oxazol-5-yl)acetamide;
N-((4-chloro-2-methylphenyl)(phenyl)methyl)-2-methyl-2-(2-(2-methylpyridin-3-yl)benzo[d]oxazol-5-yl)propanamide;
2-(4-chloro-2-methylphenyl)-N-((2-(2-methylpyridin-3-yl)benzofuran-5-yl)methyl)-2-phenylacetamide;
N-((4-chloro-2-methylphenyl)(phenyl)methyl)-2-hydroxy-2-(2-(2-methylpyridin-3-yl)benzofuran-5-yl)acetamide;
N-((4-chloro-2-methylphenyl)(phenyl)methyl)-2-(2-(2-methylpyridin-3-yl)-1-oxo-1,2,3,4-tetrahydroisoquinolin-6-yl)acetamide;
N-((4-chloro-2-methylphenyl)(phenyl)methyl)-2-(2-(2-methylpyridin-3-yl)-1H-benzo[d]imidazol-5-yl)acetamide;
N-((4-chloro-2-methylphenyl)(phenyl)methyl)-2-(2-(2-methylpyridin-3-yl)quinolin-6-yl)acetamide;
2-(2-(3,5-dimethylisoxazol-4-yl)quinolin-6-yl)-N-((2,4-dimethylphenyl)(phenyl)methyl)acetamide;
N-((4-chloro-2-methylphenyl)(phenyl)methyl)-2-(2-(2-methylpyridin-3-yl)benzo[d]thiazol-6-yl)acetamide;
N-((2,4-dimethylphenyl)(phenyl)methyl)-2-(2-(2-methylpyridin-3-yl)benzo[d]thiazol-6-yl)acetamide;
methyl ((3,5-dimethylisoxazol-4-yl)(5-(2-(((2,4-dimethylphenyl)(phenyl)methyl)amino)-2-oxoethyl)benzofuran-2-yl)methyl)carbamate;
2-(2-((3,5-dimethylisoxazol-4-yl)((2-methoxyethyl)amino)methyl)benzofuran-5-yl)-N-((2,4-dimethylphenyl)(phenyl)methyl)acetamide;
2-(2-((3,5-dimethylisoxazol-4-yl)(2-hydroxy-2-methylpropoxy)methyl)benzofuran-5-yl)-N-((2,4-dimethylphenyl)(phenyl)methyl)acetamide;
2-((3,5-dimethylisoxazol-4-yl)(5-(2-(((2,4-dimethylphenyl)(phenyl)methyl)amino)-2-oxoethyl)benzofuran-2-yl)methoxy)acetic acid;
methyl 3-((3,5-dimethylisoxazol-4-yl)(5-(2-(((2,4-dimethylphenyl)(phenyl)methyl)amino)-2-oxoethyl)benzofuran-2-yl)methoxy)propanoate;
3-((3,5-dimethylisoxazol-4-yl)(5-(2-(((2,4-dimethylphenyl)(phenyl)methyl)amino)-2-oxoethyl)benzofuran-2-yl)methoxy)propanoic acid;
2-((3,5-dimethylisoxazol-4-yl)(5-(2-(((2,4-dimethylphenyl)(phenyl)methyl)amino)-2-oxoethyl)benzofuran-2-yl)methoxy)-N,N-dimethylacetamide;
2-(2-((2-amino-2-methylpropoxy)(3,5-dimethylisoxazol-4-yl)methyl)benzofuran-5-yl)-N-((2,4-dimethylphenyl)(phenyl)methyl)acetamide;
2-((3,5-dimethylisoxazol-4-yl)(5-(2-(((2,4-dimethylphenyl)(phenyl)methyl)amino)-2-oxoethyl)benzofuran-2-yl)methoxy)-2-methylpropanoic acid;
1-((3,5-dimethylisoxazol-4-yl)(5-(2-(((2,4-dimethylphenyl)(phenyl)methyl)amino)-2-oxoethyl)benzofuran-2-yl)methyl)azetidine-3-carboxylic acid;
2-(2-((3-aminoazetidin-1-yl)(3,5-dimethylisoxazol-4-yl)methyl)benzofuran-5-yl)-N-((2,4-dimethylphenyl)(phenyl)methyl)acetamide;
N-((2,4-dimethylphenyl)(phenyl)methyl)-2-(2-(piperazin-1-yl(pyridin-4-yl)methyl)benzofuran-5-yl)acetamide;
N-(1-(3,5-dimethylpyridin-2-yl)-4-methylpentyl)-2-(2-(2-methylpyridin-3-yl)quinolin-6-yl)acetamide;
2-(2-(3,5-dimethylisoxazol-4-yl)benzofuran-5-yl)-N-(1-(2,4-dimethylphenyl)-2-isopropoxyethyl)acetamide;
N-((2,4-dimethylphenyl)(phenyl)methyl)-2-(3-(2-methylpyridin-3-yl)quinolin-6-yl)acetamide;
2-(2-((2-amino-2-oxo ethoxy)(pyridin-4-yl)methyl)benzofuran-5-yl)-N-((2,4-dimethylphenyl)(phenyl)methyl)acetamide;
2-(2-((2-amino ethoxy)(3,5-dimethylisoxazol-4-yl)methyl)benzofuran-5-yl)-N-((2,4-dimethylphenyl)(phenyl)methyl)acetamide;
N-((2,4-dimethylphenyl)(phenyl)methyl)-2-(2-(1-hydroxy-1-(pyridin-4-yl)ethyl)benzofuran-5-yl)acetamide;
4-(1-(5-(2-(((2,4-dimethylphenyl)(phenyl)methyl)amino)-2-oxoethyl)benzofuran-2-yl)-1-hydroxyethyl)pyridine 1-oxide;
N-((2,4-dimethylphenyl)(phenyl)methyl)-2-(2-(1-hydroxy-1-(2-methylpyridin-4-yl)ethyl)benzofuran-5-yl)acetamide;
N—((S)-(2,4-dimethylphenyl)(phenyl)methyl)-2-(2-(1-hydroxy-1-(3-methylpyridin-4-yl)ethyl)benzofuran-5-yl)acetamide;
2-(2-((3-amino azetidin-1-yl)(3-methylpyridin-4-yl)methyl)benzofuran-5-yl)-N—((S)-(2,4-dimethylphenyl)(phenyl)methyl)acetamide;
2-(2-((3,5-dimethylisoxazol-4-yl)methyl)-1H-benzo[d]imidazol-5-yl)-N-((2,4-dimethylphenyl)(phenyl)methyl)acetamide;
2-(2-((3,5-dimethylisoxazol-4-yl)methyl)benzo[d]oxazol-5-yl)-N-((2,4-dimethylphenyl)(phenyl)methyl)acetamide;
N-((4-chloro-2-methylphenyl)(phenyl)methyl)-2-(2-(2,4-dimethylpyridin-3-yl)benzo[d]oxazol-5-yl)acetamide;
N-((4-chloro-2-methylphenyl)(phenyl)methyl)-2-(2-(2-phenylpyridin-3-yl)benzofuran-5-yl)acetamide;
N-((2,4-dimethylphenyl)(phenyl)methyl)-2-(2-(2-methylpyridin-3-yl)-1,2,3,4-tetrahydroisoquinolin-6-yl)acetamide;
N-((2,4-dimethylphenyl)(phenyl)methyl)-2-(2-(2-methylpyridin-3-yl)-1,2,3,4-tetrahydroisoquinolin-8-yl)acetamide;
2-(2-(1-(3,5-dimethylisoxazol-4-yl)-1-hydroxyethyl)benzofuran-5-yl)-N-((2,4-dimethylphenyl)(phenyl)methyl)acetamide;
5-(5-(2-(((4-chloro-2-methylphenyl)(phenyl)methyl)amino)-2-oxoethyl)benzofuran-2-yl)nicotinic acid;
2-(2-(1-(3,5-dimethylisoxazol-4-yl)ethyl)benzofuran-5-yl)-N-((2,4-dimethylphenyl)(phenyl)methyl)acetamide;
2-(2-((3,5-dimethylisoxazol-4-yl)difluoromethyl)benzofuran-5-yl)-N-((2,4-dimethylphenyl)(phenyl)methyl)acetamide;
4-(5-(2-(((2,4-dimethylphenyl)(phenyl)methyl)amino)-2-oxoethyl)benzofuran-2-yl)-5-methylisoxazole-3-carboxylic acid;
N-((2,4-dimethylphenyl)(phenyl)methyl)-2-(3-(2-methylpyridin-3-yl)quinoxalin-6-yl)acetamide;
N-((2,4-dimethylphenyl)(phenyl)methyl)-2-(2-(2-methylpyridin-3-yl)quinoxalin-6-yl)acetamide;
2-(2-((3,5-dimethylisoxazol-4-yl)(2-methoxy-2-methylpropoxy)methyl)benzofuran-5-yl)-N-((2,4-dimethylphenyl)(phenyl)methyl)acetamide;
2-(2-(((1H-tetrazol-5-yl)methoxy)(3,5-dimethylisoxazol-4-yl)methyl)benzofuran-5-yl)-N-((2,4-dimethylphenyl)(phenyl)methyl)acetamide;
2-(2-((3,5-dimethylisoxazol-4-yl)(5-(hydroxymethyl)-1H-tetrazol-1-yl)methyl)benzofuran-5-yl)-N-((2,4-dimethylphenyl)(phenyl)methyl)acetamide;
methyl 2-((3,5-dimethylisoxazol-4-yl)(5-(2-((2,4-dimethylphenyl)(phenyl)methyl)amino)-2-oxoethyl)benzofuran-2-yl)methoxy)-2-methylpropanoate;
2-(2-((3,5-dimethylisoxazol-4-yl)(methoxy)methyl)benzofuran-5-yl)-N-((2,4-dimethylphenyl)(phenyl)methyl)acetamide;
2-(2-(((2-amino-2-oxoethyl)amino)(3,5-dimethylisoxazol-4-yl)methyl)benzofuran-5-yl)-N-((2,4-dimethylphenyl)(phenyl)methyl)acetamide;
2-(2-(3,5-dimethylisoxazol-4-yl)benzofuran-5-yl)-N-(4-methyl-1-(p-tolyl)pentyl)acetamide;
2-(1-(2-amino-2-oxoethyl)-2-(3,5-dimethylisoxazol-4-yl)-1H-benzo[d]imidazol-6-yl)-N-((2,4-dimethylphenyl)(phenyl)methyl)acetamide;
2-(1-(2-amino-2-oxoethyl)-2-(3,5-dimethylisoxazol-4-yl)-1H-benzo[d]imidazol-5-yl)-N-((2,4-dimethylphenyl)(phenyl)methyl)acetamide;
2-(2-((R)-(2-amino-2-methylpropoxy)(3,5-dimethylisoxazol-4-yl)methyl)benzofuran-5-yl)-N—((S)-(2,4-dimethylphenyl)(phenyl)methyl)acetamide;
2-(2-((S)-(2-amino-2-methylpropoxy)(3,5-dimethylisoxazol-4-yl)methyl)benzofuran-5-yl)-N—((S)-(2,4-dimethylphenyl)(phenyl)methyl)acetamide;
2-(2-((2-amino-2-methylpropoxy)(3,5-dimethylisoxazol-4-yl)methyl)benzofuran-5-yl)-N-(1-(2,4-dimethylphenyl)-4-methylpentyl)acetamide;
2-(2-((R)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide;
2-(2-((S)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide;
2-(2-((azetidin-3-yloxy)(3,5-dimethylisoxazol-4-yl)methyl)benzofuran-5-yl)-N-((2,4-dimethylphenyl)(phenyl)methyl)acetamide;
2-(2-((3,5-dimethylisoxazol-4-yl)(1H-tetrazol-1-yl)methyl)benzofuran-5-yl)-N-((2,4-dimethylphenyl)(phenyl)methyl)acetamide;
2-(2-((3,5-dimethylisoxazol-4-yl)(2H-tetrazol-2-yl)methyl)benzofuran-5-yl)-N-((2,4-dimethylphenyl)(phenyl)methyl)acetamide;
2-(2-((5-amino-2H-tetrazol-2-yl)(3,5-dimethylisoxazol-4-yl)methyl)benzofuran-5-yl)-N-((2,4-dimethylphenyl)(phenyl)methyl)acetamide;
2-(2-(((1H-tetrazol-5-yl)amino)(3,5-dimethylisoxazol-4-yl)methyl)benzofuran-5-yl)-N-((2,4-dimethylphenyl)(phenyl)methyl)acetamide;
2-(2-((R)-(3-aminoazetidin-1-yl)(3,5-dimethylisoxazol-4-yl)methyl)benzofuran-5-yl)-N—((S)-(2,4-dimethylphenyl)(phenyl)methyl)acetamide;
2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide;
2-(2-((R)-(3-aminoazetidin-1-yl)(pyridin-4-yl)methyl)benzofuran-5-yl)-N—((S)-(2,4-dimethylphenyl)(phenyl)methyl)acetamide;
2-(2-((S)-(3-aminoazetidin-1-yl)(pyridin-4-yl)methyl)benzofuran-5-yl)-N—((S)-(2,4-dimethylphenyl)(phenyl)methyl)acetamide;
ethyl 2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetate;
ethyl 2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetate;
2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetic acid;
2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetic acid;
2-(2-((3,5-dimethylisoxazol-4-yl)(methylsulfonyl)methyl)benzofuran-5-yl)-N-((2,4-dimethylphenyl)(phenyl)methyl)acetamide;
2-(2-((3,5-dimethylisoxazol-4-yl)(methylsulfonamido)methyl)benzofuran-5-yl)-N-((2,4-dimethylphenyl)(phenyl)methyl)acetamide;
N—((S)-(2,4-dimethylphenyl)(phenyl)methyl)-2-(2-(1-hydroxy-1-(pyridin-4-yl) ethyl)benzofuran-5-yl)acetamide;
4-((S)-1-(5-(2-(((S)-(2,4-dimethylphenyl)(phenyl)methyl)amino)-2-oxoethyl)benzofuran-2-yl)-1-hydroxyethyl)pyridine 1-oxide;
4-((R)-1-(5-(2-(((S)-(2,4-dimethylphenyl)(phenyl)methyl)amino)-2-oxoethyl)benzofuran-2-yl)-1-hydroxyethyl)pyridine 1-oxide;
N—((S)-(2,4-dimethylphenyl)(phenyl)methyl)-2-(2-(1-hydroxy-1-(pyrimidin-4-yl)ethyl)furo[3,2-b]pyridin-5-yl)acetamide;
N—((S)-(2,4-dimethylphenyl)(phenyl)methyl)-2-(2-(1-hydroxy-1-(2-methylpyridin-4-yl)ethyl)furo[2,3-b]pyridin-5-yl)acetamide;
1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid;
2-(2-((3-amino azetidin-1-yl)(3,5-dimethylisoxazol-4-yl)methyl)benzofuran-5-yl)-N-((4-hydroxy-2-methylphenyl)(phenyl)methyl)acetamide;
2-(2-((3-aminoazetidin-1-yl)(3,5-dimethylisoxazol-4-yl)methyl)benzofuran-5-yl)-N-((4-methoxy-2-methylphenyl)(phenyl)methyl)acetamide;
2-(2-((2-amino-2-methylpropoxy)(3,5-dimethylisoxazol-4-yl)methyl)benzofuran-6-yl)-N-((4-hydroxy-2-methylphenyl)(phenyl)methyl)acetamide;
1-((R)-(3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid;
1-((S)-(3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid;
2-(2-((R)-(3-amino azetidin-1-yl)(3,5-dimethylisoxazol-4-yl)methyl)benzofuran-5-yl)-N—((S)-(2,4-dimethylphenyl)(phenyl)methyl)acetamide;
2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide;
2-(2-((3,5-dimethylisoxazol-4-yl)(3-(methylsulfonyl)azetidin-1-yl)methyl)furo[3,2-b]pyridin-5-yl)-N—((S)-(2,4-dimethylphenyl)(phenyl)methyl)acetamide;
1-(1-(4-fluorophenyl)-6-methyl-3,4-dihydroisoquinolin-2(1H)-yl)-2-(2-(1-hydroxy-1-(pyridin-4-yl)ethyl)benzofuran-5-yl)ethanone;
4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoic acid;
4-(3,5-dimethylisoxazol-4-yl)-4-(5-(2-(((2,4-dimethylphenyl)(phenyl)methyl)amino)-2-oxoethyl)benzofuran-2-yl)butanoic acid;
1-((4-chlorophenyl)(5-(2-(((S)-(2,4-dimethylphenyl)(phenyl)methyl)amino)-2-oxoethyl)benzofuran-2-yl)methoxy)cyclopropanec arb oxylic acid;
2-(2-((4-chlorophenyl)(methoxy)methyl)benzofuran-5-yl)-N—((S)-(2,4-dimethylphenyl)(phenyl)methyl)acetamide;
N-((2,4-dimethylphenyl)(phenyl)methyl)-2-(2-(1-hydroxy-1-(2-hydroxypyridin-4-yl)ethyl)furo[3,2-b]pyridin-5-yl)acetamide;
1-((3,5-dimethylisoxazol-4-yl)(5-(2-(((S)-(2,4-dimethylphenyl)(phenyl)methyl)amino)-2-oxoethyl)-7-fluorobenzofuran-2-yl)methoxy)cyclopropanecarboxylic acid;
1-((2,4-dimethyloxazol-5-yl)(5-(2-(((S)-(2,4-dimethylphenyl)(phenyl)methyl)amino)-2-oxoethyl)benzofuran-2-yl)methoxy)cyc lopropanec arb oxylic acid;
2-(2-(3,5-dimethylisoxazol-4-yl)-1H-benzo[d]imidazol-6-yl)-N-((2,4-dimethylphenyl)(phenyl)methyl)acetamide;
2-(2-((2-amino-2-methylpropoxy)(pyridin-4-yl)methyl)benzofuran-5-yl)-N-((2,4-dimethylphenyl)(phenyl)methyl)acetamide;
N-((4-chloro-2-methylphenyl)(pyridin-2-yl)methyl)-2-(2-(3,5-dimethylisoxazol-4-yl)benzofuran-5-yl)acetamide;
N-((4-cyano-2-methylphenyl)(phenyl)methyl)-2-(2-(1-hydroxy-1-(pyridin-4-yl)ethyl)benzofuran-5-yl)acetamide;
3-(5-(2-(((S)-(2,4-dimethylphenyl)(phenyl)methyl)amino)-2-oxoethyl)benzofuran-2-yl)-3-(pyridin-4-yl)propanoic acid;
N—((S)-(2,4-dimethylphenyl)(phenyl)methyl)-2-(2-(2,2,2-trifluoro-1-hydroxy-1-(pyridin-4-yl)ethyl)benzofuran-5-yl)acetamide;
2-(2-(1-hydroxy-1-(pyridin-4-yl)ethyl)benzofuran-5-yl)-N-((3-methylpyridin-4-yl)(phenyl)methyl)acetamide;
2-(2-((3,5-dimethylisoxazol-4-yl)(hydroxy)methyl)benzofuran-5-yl)-1-(5-phenyl-2,3-dihydrobenzo[f][1,4]oxazepin-4(5H)-yl)ethanone;
2-(2-(1-(3,5-dimethylisoxazol-4-yl)-2-hydroxyethyl)benzofuran-5-yl)-N—((S)-(2,4-dimethylphenyl)(phenyl)methyl)acetamide;
4-((R)-1-hydroxy-1-(5-(2-((R)-6-methyl-1-phenyl-3,4-dihydroisoquinolin-2(1H)-yl)-2-oxoethyl)benzofuran-2-yl)ethyl)pyridine 1-oxide;
4-((R)-1-hydroxy-1-(5-(2-((S)-6-methyl-1-phenyl-3,4-dihydroisoquinolin-2(1H)-yl)-2-oxoethyl)benzofuran-2-yl)ethyl)pyridine 1-oxide;
4-((S)-1-hydroxy-1-(5-(2-((R)-6-methyl-1-phenyl-3,4-dihydroisoquinolin-2(1H)-yl)-2-oxoethyl)benzofuran-2-yl)ethyl)pyridine 1-oxide;
4-((S)-1-hydroxy-1-(5-(2-((S)-6-methyl-1-phenyl-3,4-dihydroisoquinolin-2(1H)-yl)-2-oxoethyl)benzofuran-2-yl)ethyl)pyridine 1-oxide;
1-(1,8-dimethyl-5-phenyl-2,3-dihydro-1H-benzo[e][1,4]diazepin-4(5H)-yl)-2-(2-(1-hydroxy-1-(pyridin-4-yl)ethyl)benzofuran-5-yl)ethanone;
2-(2-(3,5-dimethylisoxazol-4-yl)benzofuran-5-yl)-N-(1-(2-methoxy-4-methylphenyl)-4-methylpentyl)acetamide;
N-((4-chloro-2-methylphenyl)(phenyl)methyl)-2-(2-(3,5-dimethylisoxazol-4-yl)benzofuran-5-yl)acetamide;
2-(2-(3,5-dimethylisoxazol-4-yl)benzofuran-5-yl)-N-(1-(2,6-dimethylpyridin-3-yl)-4-methylpentyl)acetamide;
2-(2-(3,5-dimethylisoxazol-4-yl)benzofuran-5-yl)-N-(4-methyl-1-(4-methyl-2-(trifluoromethyl)phenyl)pentyl)acetamide;
N-(1-(2,4-dichlorophenyl)-4-methylpentyl)-2-(2-(3,5-dimethylisoxazol-4-yl)benzofuran-5-yl)acetamide;
N-(1-(4-chloro-2,6-difluorophenyl)-4-methylpentyl)-2-(2-(3,5-dimethylisoxazol-4-yl)benzofuran-5-yl)acetamide;
N-((2,4-dimethylphenyl)(phenyl)methyl)-2-(2-(pyridin-3-yl)benzofuran-5-yl)acetamide;
N-((4-chloro-2-methylphenyl)(phenyl)methyl)-2-(2-(pyridin-3-yl)benzofuran-5-yl)acetamide;
2-(2-((S)-(3,5-dimethylisoxazol-4-yl)(hydroxy)methyl)benzofuran-5-yl)-1-(6-methyl-1-phenyl-3,4-dihydroisoquinolin-2(1H)-yl)ethanone;
N—((S)-(2,4-dimethylphenyl)(phenyl)methyl)-2-(2-(1-hydroxy-1-(pyridin-3-yl)ethyl)benzofuran-5-yl)acetamide;
N—((S)-(2,4-dimethylphenyl)(phenyl)methyl)-2-(2-(1-(3-fluoropyridin-4-yl)-1-hydroxyethyl)benzofuran-5-yl)acetamide;
1-(6-chloro-1-phenyl-3,4-dihydroisoquinolin-2(1H)-yl)-2-(2-(1-(3-fluoropyridin-4-yl)-1-hydroxyethyl)benzofuran-5-yl)ethanone;
1-(8-chloro-1-phenyl-3,4-dihydroisoquinolin-2(1H)-yl)-2-(2-(1-(3-fluoropyridin-4-yl)-1-hydroxyethyl)benzofuran-5-yl)ethanone;
N-((2,4-dimethylphenyl)(4-fluorophenyl)methyl)-2-(2-(1-hydroxy-1-(pyridin-4-yl)ethyl)benzofuran-5-yl)acetamide;
2-(2-(1-hydroxy-1-(pyridin-4-yl)ethyl)benzofuran-5-yl)-1-(6-methoxy-1-phenyl-3,4-dihydroisoquinolin-2(1H)-yl)ethanone;
1-(6-chloro-1-phenyl-3,4-dihydroisoquinolin-2(1H)-yl)-2-(2-(1-hydroxy-1-(pyridin-4-yl)ethyl)benzofuran-5-yl)ethanone;
1-(8-chloro-1-phenyl-3,4-dihydroisoquinolin-2(1H)-yl)-2-(2-(1-hydroxy-1-(pyridin-4-yl)ethyl)benzofuran-5-yl)ethanone;
2-(2-(1-(3-fluoropyridin-4-yl)-1-hydroxyethyl)benzofuran-5-yl)-N-((4-(hydroxymethyl)-2-methylphenyl)(phenyl)methyl)acetamide;
2-(2-(1-(3-fluoropyridin-4-yl)-1-hydroxyethyl)benzofuran-5-yl)-N-((4-methoxy-2-methylphenyl)(phenyl)methyl)acetamide;
1-(1-(4-fluorophenyl)-6-methyl-3,4-dihydroisoquinolin-2(1H)-yl)-2-(2-(1-(3-fluoropyridin-4-yl)-1-hydroxyethyl)benzofuran-5-yl)ethanone;
N-((4-chloro-2-methylphenyl)(phenyl)methyl)-2-(2-(1-(3-fluoropyridin-4-yl)-1-hydroxyethyl)benzofuran-5-yl)acetamide;
2-(2-(1-hydroxy-1-(pyridin-4-yl)ethyl)benzofuran-5-yl)-N-((4-(hydroxymethyl)-2-methylphenyl)(phenyl)methyl)acetamide;
2-(2-(1-(3-fluoropyridin-4-yl)-1-hydroxyethyl)benzofuran-5-yl)-1-(6-methoxy-1-phenyl-3,4-dihydroisoquinolin-2(1H)-yl)ethanone;
N-((4-chlorophenyl)(2,4-dimethylphenyl)methyl)-2-(2-(pyridin-3-yl)benzofuran-5-yl)acetamide;
N-((4-chloro-2-methylphenyl)(phenyl)methyl)-2-(2-(2-(trifluoromethyl)pyridin-3-yl)benzofuran-5-yl)acetamide;
N-((4-chloro-2-methylphenyl)(phenyl)methyl)-2-methyl-2-(2-(pyrimidin-5-yl)benzofuran-5-yl)propanamide;
N-((4-chloro-2-methylphenyl)(phenyl)methyl)-2-methyl-2-(2-(2-methylpyridin-3-yl)benzofuran-5-yl)propanamide;
N-((2,4-dimethylphenyl)(phenyl)methyl)-2-(2-(pyridin-4-yl)benzofuran-5-yl)acetamide;
2-(2-(3,5-dimethylisoxazol-4-yl)benzofuran-5-yl)-N-((2,4-dimethylphenyl)(phenyl)methyl)acetamide;
2-(2-(3,5-dimethylisoxazol-4-yl)benzofuran-5-yl)-N-((4-methoxyphenyl) (phenyl)methyl)acetamide;
2-(2-(3,5-dimethylisoxazol-4-yl)benzofuran-5-yl)-N-((2,4-dimethylphenyl)(4-fluorophenyl)methyl)acetamide;
2-(2-(3,5-dimethylisoxazol-4-yl)benzo[d]oxazol-5-yl)-N-((4-methoxyphenyl)(phenyl)methyl)acetamide;
2-(2-(3,5-dimethylisoxazol-4-yl)benzo[d]oxazol-5-yl)-N-((2,4-dimethylphenyl)(4-fluorophenyl)methyl)acetamide;
N-((4-chloro-2-methylphenyl)(phenyl)methyl)-2-(2-(3,5-dimethylisoxazol-4-yl)benzo[d]oxazol-5-yl)acetamide;
N-((4-chloro-2-methylphenyl)(phenyl)methyl)-2-(2-(2-methylpyridin-3-yl)benzofuran-5-yl)acetamide;
N-((4-chloro-2-methylphenyl)(phenyl)methyl)-2-(2-(2-methylpyridin-3-yl)benzo[d]oxazol-5-yl)acetamide;
N-((4-chloro-2-methylphenyl)(phenyl)methyl)-2-(2-(4,6-dimethylpyrimidin-5-yl)benzo[d]oxazol-5-yl)acetamide;
N-((4-chloro-2-methylphenyl)(phenyl)methyl)-2-(2-(2-hydroxypyridin-3-yl)benzo[d]oxazol-5-yl)acetamide;
N-((2,4-dimethylphenyl)(phenyl)methyl)-2-(2-(pyridin-4-yl)benzo[d]oxazol-5-yl)acetamide;
N-((2,4-dimethylphenyl)(phenyl)methyl)-2-(2-(2-methylpyridin-3-yl)quinolin-6-yl)acetamide;
2-(2-((3,5-dimethylisoxazol-4-yl)(neopentyloxy)methyl)benzofuran-5-yl)-N-((2,4-dimethylphenyl)(phenyl)methyl)acetamide;
2-(2-((3,5-dimethylisoxazol-4-yl)(2-methoxyethoxy)methyl)benzofuran-5-yl)-N-((2,4-dimethylphenyl)(phenyl)methyl)acetamide;
2-(2-((3,5-dimethylisoxazol-4-yl)(4-hydroxypiperidin-1-yl)methyl)benzofuran-5-yl)-N-((2,4-dimethylphenyl)(phenyl)methyl)acetamide;
2-(2((3,5-dimethylisoxazol-4-yl)(3,3-dimethylpiperazin-1-yl)methyl)benzofuran-5-yl)-N-((2,4-dimethylphenyl)(phenyl)methyl)acetamide;
2-(2-((3,5-dimethylisoxazol-4-yl)(morpholino)methyl)benzofuran-5-yl)-N-((2,4-dimethylphenyl)(phenyl)methyl)acetamide;
2-(((3,5-dimethylisoxazol-4-yl)(5-(2-(((2,4-dimethylphenyl)(phenyl)methyl)amino)-2-oxoethyl)benzofuran-2-yl)methyl)amino)acetic acid;
2-(2-((3,5-dimethylisoxazol-4-yl)((2-hydroxyethyl)amino)methyl)benzofuran-5-yl)-N-((2,4-dimethylphenyl)(phenyl)methyl)acetamide;
2-((3,5-dimethylisoxazol-4-yl)(5-(2-(((2,4-dimethylphenyl)(phenyl)methyl)amino)-2-oxoethyl)benzofuran-2-yl)methyl)amino)-N,N-dimethylacetamide;
2-(2-((3-amino-3-methylbutoxy)(3,5-dimethylisoxazol-4-yl)methyl)benzofuran-5-yl)-N-((2,4-dimethylphenyl)(phenyl)methyl)acetamide;
2-((5-(2-(((2,4-dimethylphenyl)(phenyl)methyl)amino)-2-oxoethyl)benzofuran-2-yl)(2-methylpyridin-3-yl)methoxy)acetic acid;
tert-butyl 2-((3,5-dimethylisoxazol-4-yl)(5-(2-(((2,4-dimethylphenyl)(phenyl)methyl)amino)-2-oxoethyl)benzofuran-2-yl)methoxy)acetate;
2-(2-((2-amino-2-oxoethoxy)(3,5-dimethylisoxazol-4-yl)methyl)benzofuran-5-yl)-N-((2,4-dimethylphenyl)(phenyl)methyl)acetamide;
2-(2-((2-amino-2-oxoethoxy)(3,5-dimethylisoxazol-4-yl)methyl)benzofuran-5-yl)-N-(1-(2,4-dimethylphenyl)-4-methylpentyl)acetamide;
2-((S)-(3,5-dimethylisoxazol-4-yl)(5-(2-((S)-(2,4-dimethylphenyl)(phenyl)methyl)amino)-2-oxoethyl)benzofuran-2-yl)methoxy)acetic acid;
N-((2,4-dimethylphenyl)(phenyl)methyl)-2-(2-(3-methylpyridin-4-yl)benzofuran-5-yl)acetamide;
2-(2-((2-amino-2-methylpropoxy)(3,5-dimethylisoxazol-4-yl)methyl)benzofuran-5-yl)-N-(1-(3,5-dimethylpyridin-2-yl)-4-methylpentyl)acetamide;
2-((3,5-dimethylisoxazol-4-yl)(5-(2-((1-(3,5-dimethylpyridin-2-yl)-4-methylpentyl)amino)-2-oxoethyl)benzofuran-2-yl)methoxy)-2-methylpropanoic acid;
2-((3,5-dimethylisoxazol-4-yl)(5-(2-((1-(2,4-dimethylphenyl)-4-methylpentyl)amino)-2-oxoethyl)benzofuran-2-yl)methoxy)-2-methylpropanoic acid;
2-((5-(2-(((2,4-dimethylphenyl)(phenyl)methyl)amino)-2-oxoethyl)benzofuran-2-yl)(pyridin-4-yl)methoxy)acetic acid;
2-((S)-(3,5-dimethylisoxazol-4-yl)(5-(2-(((S)-(2,4-dimethylphenyl)(phenyl)methyl)amino)-2-oxoethyl)benzofuran-2-yl)methoxy)-2-methylpropanoic acid;
2-((R)-(3,5-dimethylisoxazol-4-yl)(5-(2-(((R)-(2,4-dimethylphenyl)(phenyl)methyl)amino)-2-oxoethyl)benzofuran-2-yl)methoxy)-2-methylpropanoic acid;
2-(((S)-(3,5-dimethylisoxazol-4-yl)(5-(2-(((R)-(2,4-dimethylphenyl)(phenyl)methyl)amino)-2-oxoethyl)benzofuran-2-yl)methoxy)-2-methylpropanoic acid;
2-((R)-(3,5-dimethylisoxazol-4-yl)(5-(2-(((S)-(2,4-dimethylphenyl)(phenyl)methyl)amino)-2-oxoethyl)benzofuran-2-yl)methoxy)-2-methylpropanoic acid;
2-((5-(2-(((2,4-dimethylphenyl)(phenyl)methyl)amino)-2-oxoethyl)benzofuran-2-yl)(pyridin-4-yl)methoxy)-2-methylpropanoic acid;
N-((2,4-dimethylphenyl)(phenyl)methyl)-2-(2-(((2-hydroxy-2-methylpropyl)amino)(pyridin-4-yl)methyl)benzofuran-5-yl)acetamide;
2-(2-((3-amino-3-methylazetidin-1-yl)(3,5-dimethylisoxazol-4-yl)methyl)benzofuran-5-yl)-N-((2,4-dimethylphenyl)(phenyl)methyl)acetamide;
2-(2-((4-aminopiperidin-1-yl)(3,5-dimethylisoxazol-4-yl)methyl)benzofuran-5-yl)-N-((2,4-dimethylphenyl)(phenyl)methyl)acetamide;
2-(2-((3-aminoazetidin-1-yl)(3,5-dimethylisoxazol-4-yl)methyl)benzofuran-5-yl)-N-((4-chloro-2-methylphenyl)(phenyl)methyl)acetamide;
2-(2-((3-aminoazetidin-1-yl)(3-fluoropyridin-4-yl)methyl)benzofuran-5-yl)-N—((S)-(2,4-dimethylphenyl)(phenyl)methyl)acetamide;
2-(2-((3-aminoazetidin-1-yl)(pyridin-4-yl)methyl)benzofuran-5-yl)-N-((2,4-dimethylphenyl)(phenyl)methyl)acetamide;
2-(2-((3-aminoazetidin-1-yl)(3,5-dimethylisoxazol-4-yl)methyl)benzofuran-5-yl)-N-((4-methyl-2-(trifluoromethyl)phenyl)(phenyl)methyl)acetamide;
2-(2-((3-aminoazetidin-1-yl)(3,5-dimethylisoxazol-4-yl)methyl)benzofuran-5-yl)-N-((2-(dimethylamino)-4-methylphenyl)(phenyl)methyl)acetamide;
2-(2-((3-amino azetidin-1-yl)(3,5-dimethylisoxazol-4-yl)methyl)benzofuran-5-yl)-N-(4-methyl-1-(4-methyl-2-(trifluoromethyl)phenyl)pentyl)acetamide;
2-(2-((3-amino azetidin-1-yl)(3,5-dimethylisoxazol-4-yl)methyl)benzofuran-5-yl)-N-((2,4-dimethylphenyl)(pyridin-2-yl)methyl)acetamide;
2-(2-((3-amino azetidin-1-yl)(3,5-dimethylisoxazol-4-yl)methyl)benzofuran-5-yl)-N-((2-methoxy-4-methylphenyl)(phenyl)methyl)acetamide;
2-(2-(3,5-dimethylisoxazol-4-yl)benzofuran-5-yl)-N-(1-(3,5-dimethylpyridin-2-yl)-4-methylpentyl)acetamide;
N-(1-(2-(dimethylamino)-4-methylphenyl)-4-methylpentyl)-2-(2-(3,5-dimethylisoxazol-4-yl)benzofuran-5-yl)acetamide;
N-(1-(2,4-dimethylphenyl)-4-methylpentyl)-2-(2-(2-methylpyridin-3-yl)quinolin-6-yl)acetamide;
2-(2-(((2-amino-2-methylpropyl)amino)(3,5-dimethylisoxazol-4-yl)methyl)benzofuran-5-yl)-N-((2,4-dimethylphenyl)(phenyl)methyl)acetamide;
2-(2-((3,5-dimethylisoxazol-4-yl)(piperazin-1-yl)methyl)benzofuran-5-yl)-N-((2,4-dimethylphenyl)(phenyl)methyl)acetamide;
2-(2-((3,5-dimethylisoxazol-4-yl)(piperazin-1-yl)methyl)benzofuran-5-yl)-N-(1-(3,5-dimethylpyridin-2-yl)-4-methylpentyl)acetamide;
2-(2-((3,5-dimethylisoxazol-4-yl)(2-methyl-2-(methylamino)prop oxy)methyl)benzofuran-5-yl)-N-((2,4-dimethylphenyl)(phenyl)methyl)acetamide;
N-((4-chloro-2-methylphenyl)(phenyl)methyl)-2-(2-(pyrimidin-5-yl)benzo[d]oxazol-5-yl)acetamide;
N-((4-chloro-2-methylphenyl)(phenyl)methyl)-2-(2-(2-methylpyridin-3-yl)benzo[d]oxazol-5-yl)acetamide;
N-((4-chloro-2-methylphenyl)(phenyl)methyl)-2-(2-(4,6-dimethylpyrimidin-5-yl)benzo[d]oxazol-5-yl)acetamide;
N-((4-chloro-2-methylphenyl)(phenyl)methyl)-2-(2-(2-oxo-1,2-dihydropyridin-3-yl)benzo[d]oxazol-5-yl)acetamide;
methyl 4-(5-(2-(((2,4-dimethylphenyl)(phenyl)methyl)amino)-2-oxoethyl)benzofuran-2-yl)-5-methylisoxazole-3-carboxylate;
2-((3,5-dimethylisoxazol-4-yl)(5-(2-(((2,4-dimethylphenyl)(phenyl)methyl)amino)-2-oxoethyl)benzofuran-2-yl)methoxy)-2-methylpropanamide;
2-((5-(2-(((2,4-dimethylphenyl)(phenyl)methyl)amino)-2-oxoethyl)benzofuran-2-yl)(pyridin-4-yl)methoxy)-2-methylpropanamide;
4-(1-(5-(2-(((S)-(2,4-dimethylphenyl)(phenyl)methyl)amino)-2-oxoethyl)benzofuran-2-yl)-1-hydroxyethyl)pyridine 1-oxide;
4-(1-(5-(2-(((S)-(2,4-dimethylphenyl)(phenyl)methyl)amino)-2-oxoethyl)benzofuran-2-yl)-1-hydroxyethyl)pyrimidine 1-oxide;
4-(1-(5-(2-(((S)-(2,4-dimethylphenyl)(phenyl)methyl)amino)-2-oxoethyl)benzofuran-2-yl)-1-hydroxyethyl)-2-methylpyridine 1-oxide;
4-(1-(5-(2-(1-(4-fluorophenyl)-6-methyl-3,4-dihydroisoquinolin-2(1H)-yl)-2-oxoethyl)benzofuran-2-yl)-1-hydroxyethyl)pyridine 1-oxide;
4-(1-hydroxy-1-(5-(2-(((4-methoxy-2-methylphenyl)(phenyl)methyl)amino)-2-oxoethyl)benzofuran-2-yl)ethyl)pyridine 1-oxide;
4-(1-(6-(2-(((4-chloro-2-methylphenyl)(phenyl)methyl)amino)-2-oxoethyl)benzofuran-2-yl)-1-hydroxyethyl)pyridine 1-oxide;
4-(1-hydroxy-1-(5-(2-(8-methyl-1-phenyl-3,4-dihydroisoquinolin-2(1H)-yl)-2-oxoethyl)benzofuran-2-yl)ethyl)pyridine 1-oxide;
4-(1-hydroxy-1-(5-(2-(((2-methoxyphenyl)(phenyl)methyl)amino)-2-oxoethyl)benzofuran-2-yl)ethyl)pyridine 1-oxide;
4-(1-hydroxy-1-(5-(2-(6-methyl-1-phenyl-3,4-dihydroisoquinolin-2(1H)-yl)-2-oxoethyl)benzofuran-2-yl)ethyl)pyridine 1-oxide;
4-(1-hydroxy-1-(5-(2-oxo-2-((phenyl(o-tolyl)methyl)amino)ethyl)benzofuran-2-yl)ethyl)pyridine 1-oxide;
3-(1-(5-(2-(((S)-(2,4-dimethylphenyl)(phenyl)methyl)amino)-2-oxoethyl)benzofuran-2-yl)-1-hydroxyethyl)pyridine 1-oxide;
4-(1-(5-(2-(((S)-(2,4-dimethylphenyl)(phenyl)methyl)amino)-2-oxoethyl)benzofuran-2-yl)-1-hydroxyethyl)-3-fluoropyridine 1-oxide;
4-(1-(5-(2-(((2,4-dimethylphenyl)(4-fluorophenyl)methyl)amino)-2-oxoethyl)benzofuran-2-yl)-1-hydroxyethyl)-3-fluoropyridine 1-oxide;
4-(1-(5-(2-(6-chloro-1-phenyl-3,4-dihydroisoquinolin-2(1H)-yl)-2-oxoethyl)benzofuran-2-yl)-1-hydroxyethyl)-3-fluoropyridine 1-oxide;
4-(1-(5-(2-(((2,4-dimethylphenyl)(4-fluorophenyl)methyl)amino)-2-oxoethyl)benzofuran-2-yl)-1-hydroxyethyl)pyridine 1-oxide;
4-(1-hydroxy-1-(5-(2-(6-methoxy-1-phenyl-3,4-dihydroisoquinolin-2(1H)-yl)-2-oxoethyl)benzofuran-2-yl)ethyl)pyridine 1-oxide;
4-(1-(5-(2-(6-chloro-1-phenyl-3,4-dihydroisoquinolin-2(1H)-yl)-2-oxoethyl)benzofuran-2-yl)-1-hydroxyethyl)pyridine 1-oxide;
4-(1-(5-(2-(8-chloro-1-phenyl-3,4-dihydroisoquinolin-2(1H)-yl)-2-oxoethyl)benzofuran-2-yl)-1-hydroxyethyl)pyridine 1-oxide;
3-fluoro-4-(1-hydroxy-1-(5-(2-(((4-(hydroxymethyl)-2-methylphenyl)(phenyl)methyl)amino)-2-oxoethyl)benzofuran-2-yl)ethyl)pyridine 1-oxide;
3-fluoro-4-(1-hydroxy-1-(5-(2-(((4-methoxy-2-methylphenyl)(phenyl)methyl)amino)-2-oxoethyl)benzofuran-2-yl)ethyl)pyridine 1-oxide;
3-fluoro-4-(1-(5-(2-(1-(4-fluorophenyl)-6-methyl-3,4-dihydroisoquinolin-2(1H)-yl)-2-oxoethyl)benzofuran-2-yl)-1-hydroxyethyl)pyridine 1-oxide;
4-(1-(5-(2-(((4-chloro-2-methylphenyl)(phenyl)methyl)amino)-2-oxoethyl)benzofuran-2-yl)-1-hydroxyethyl)-3-fluoropyridine 1-oxide;
4-(1-hydroxy-1-(5-(2-(((4-(hydroxymethyl)-2-methylphenyl)(phenyl)methyl)amino)-2-oxoethyl)benzofuran-2-yl)ethyl)pyridine 1-oxide;
3-fluoro-4-(1-hydroxy-1-(5-(2-(6-methoxy-1-phenyl-3,4-dihydroisoquinolin-2(1H)-yl)-2-oxoethyl)benzofuran-2-yl)ethyl)pyridine 1-oxide;
N—((S)-(2,4-dimethylphenyl)(phenyl)methyl)-2-(2-(1-hydroxy-1-(2-methylpyridin-4-yl)ethyl)furo[3,2-b]pyridin-5-yl)acetamide;
N—((S)-(2,4-dimethylphenyl)(phenyl)methyl)-2-(2-(1-hydroxy-1-(3-methylpyridin-4-yl)ethyl)furo[3,2-b]pyridin-5-yl)acetamide;
N—((S)-(2,4-dimethylphenyl)(phenyl)methyl)-2-(2-(1-hydroxy-1-(6-methylpyrimidin-4-yl)ethyl)benzofuran-5-yl)acetamide;
N—((S)-(2,4-dimethylphenyl)(phenyl)methyl)-2-(2-(1-hydroxy-1-(pyridin-4-yl)ethyl)furo[3,2-b]pyridin-5-yl)acetamide;
N—((S)-(2,4-dimethylphenyl)(phenyl)methyl)-2-(2-(1-hydroxy-1-(2-methylpyridin-4-yOethyl)benzofuran-5-yl)acetamide;
N—((S)-(2,4-dimethylphenyl)(phenyl)methyl)-2-(2-(1-hydroxy-1-(pyrimidin-4-yl)ethyl)benzofuran-5-yl)acetamide;
N—((S)-(2,4-dimethylphenyl)(phenyl)methyl)-2-(2-(1-hydroxy-1-(2-methylpyrimidin-4-yl)ethyl)benzofuran-5-yl)acetamide;
N-((4-chloro-2-methylphenyl)(phenyl)methyl)-2-(2-(1-hydroxy-1-(pyridin-4-yl)ethyl)benzofuran-6-yl)acetamide;
2-(2-(1-hydroxy-1-(pyridin-4-yl)ethyl)benzofuran-5-yl)-1-(8-methyl-1-phenyl-3,4-dihydroisoquinolin-2(1H)-yl)ethanone;
N-(4-(2,4-dimethylphenyl)tetrahydro-2H-pyran-4-yl)-2-(2-(1-hydroxy-1-(pyridin-4-yl)ethyl)benzofuran-5-yl)acetamide;
2-(2-(1-hydroxy-1-(pyridin-4-yl)ethyl)benzofuran-5-yl)-N-((2-methoxyphenyl)(phenyl)methyl)acetamide;
2-(2-(1-hydroxy-1-(pyridin-4-yl)ethyl)benzofuran-5-yl)-1-(6-methyl-1-phenyl-3,4-dihydroisoquinolin-2(1H)-yl)ethanone;
2-(2-(1-hydroxy-1-(pyridin-4-yl)ethyl)benzofuran-5-yl)-N-(phenyl(o-tolyl)methyl)acetamide;
N-((2,4-dimethylphenyl)(4-fluorophenyl)methyl)-2-(2-(1-(3-fluoropyridin-4-yl)-1-hydroxyethyl)benzofuran-5-yl)acetamide;
2-(2-(1-(4-chlorophenyl)-1-hydroxyethyl)benzofuran-5-yl)-N—((S)-(2,4-dimethylphenyl)(phenyl)methyl)acetamide;
2-(2-((3,5-dimethylisoxazol-4-yl)((2-hydroxy-2-methylpropyl)amino)methyl)benzofuran-5-yl)-N-((2,4-dimethylphenyl)(phenyl)methyl)acetamide;

or pharmaceutically acceptable salts thereof.

A particular embodiment of a deuterated compound of Formula (I) is 4-(1-(5-(2-((deutero-(2,4-dimethylphenyl)(phenyl)methyl)amino)-2-oxoethyl)benzofuran-2-yl)-1-hydroxyethyl)pyridine 1-oxide.

The meaning of any functional group or substituent thereon at any one occurrence in Formula (I), or any subformula thereof, is independent of its meaning, or any other functional group's or substituent's meaning, at any other occurrence, unless stated otherwise.

It is to be understood that in a compound of Formula (I) wherein m is 2, each instance of R³ is selected independently from the other R³. Similarly, in a compound of Formula (I) wherein m is 2, each instance of R^{3a} is selected independently from the other R^{3a}. It is to be further understood that in a compound of Formula (I) wherein n is 2, each instance of R⁴ is selected independently from the other R⁴. Similarly, in a compound of Formula (I) wherein n is 2, each instance of R^{4a} is selected independently from the other R^{4a}.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula (I), or in any chemical structure illustrated herein, is not specified the structure is intended to encompass all individual stereoisomers and all mixtures thereof. Thus, compounds according to Formula (I) containing one or more chiral center may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” means products whose enantiomeric excess is 99% ee or greater.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of Formula (I), or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula (I) are preferably pharmaceutically acceptable. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of Formula (I).

Salts of the compounds of Formula (I) containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the compounds of Formula (I) containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other non-pharmaceutically acceptable salts, e.g. trifluoroacetate, may be used, for example in the isolation of compounds of the invention, and are included within the scope of this invention.

The invention includes within its scope all possible stoichiometric and non-stoichiometric forms of the salts of the compounds of Formula (I).

If a compound of Formula (I) containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of Formula (I) containing a carboxylic acid or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

The invention also includes various deuterated forms of the compounds of Formula (I), for example, the compound of Example 225, 4-(1-(5-(2-((deutero-(2,4-dimethylphenyl) (phenyl)methyl)amino)-2-oxoethyl)benzofuran-2-yl)-1-hydroxyethyl)pyridine 1-oxide. Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride or sodium borodeuteride).

### Methods of Use

Modulators of RORγ can be useful in the treatment of diseases mediated by RORγ, particularly autoimmune or inflammatory diseases and cancer. Such inflammatory or autoimmune diseases include multiple sclerosis, rheumatoid arthritis, psoriasis, Crohn's disease, inflammatory bowel disease, graft-versus-host disease (GVHD), Sjorgen's syndrome, optic neuritis, chronic obstructive pulmonary disease, asthma, type I diabetes, neuromyelitis optica, myasthenia gravis, uveitis, Behcets disease, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, dry eye and glomerulonephritis, myocarditis, especially psoriasis Such cancers include multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer. Accordingly, in another aspect the invention is directed to methods of treating such diseases using a compound of Formula (I) or a pharmaceutically acceptable salt thereof. The methods of treatment of the invention comprise administering an effective amount of a compound according to Formula (I) or a pharmaceutically acceptable salt thereof to a patient (particularly a human) in need thereof.

In a further aspect, the invention is directed to a compound of Formula (I) or a pharmaceutically acceptable salt thereof for use in therapy. In particular, for use in the treatment of diseases mediated by RORγ, particularly autoimmune or inflammatory diseases and cancer, such as those disclosed above.

In a further aspect, the invention is directed to the use of a compound of Formula (I) or a pharmaceutically acceptable salt thereof in the manufacture of a medicament for the treatment of diseases mediated by RORγ, particularly autoimmune or inflammatory diseases and cancer, such as those disclosed above.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” refers to a human or a mammal, especially a human.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

It will be appreciated by those skilled in the art that certain protected derivatives of compounds of Formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention.

Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred “pro-moieties” for compounds of the invention include: ester, carbonate ester, hemi-ester, phosphate ester, nitro ester, sulfate ester, sulfoxide, amide, carbamate, azo-, phosphamide, glycoside, ether, acetal, and ketal derivatives of the compounds of Formula (I).

Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound.

The invention includes the use of compounds of the invention for the preparation of a composition for treating or ameliorating diseases mediated by RORγ in a subject in need thereof, wherein the composition comprises a mixture of one or more of the compounds of the invention and an optional pharmaceutically acceptable excipient.

The invention further includes the use of compounds of the invention as an active therapeutic substance, in particular in the treatment of diseases mediated by RORγ. In another embodiment, the invention relates to the use of compounds of the invention in the preparation of a medicament for the treatment of diseases mediated by RORγ.

Examples of such diseases include autoimmune or inflammatory diseases such as multiple sclerosis, rheumatoid arthritis, psoriasis, Crohn's disease, inflammatory bowel disease, Sjorgen's syndrome, optic neuritis, chronic obstructive pulmonary disease, asthma, type I diabetes, neuromyelitis optica, Myasthenia Gravis, uveitis, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, Dry Eye, glomerulonephritis, myocarditis and cancer diseases including multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer.

The invention includes the use of compounds of the invention for the preparation of a composition for treating or ameliorating diseases mediated by RORγ in a subject in need thereof, wherein the composition comprises a mixture of one or more of the compounds of the invention and an optional pharmaceutically acceptable excipient.

The compounds of the invention may be used alone or in combination with one or more other therapeutic agents. Accordingly the present invention provides a combination comprising a compound of Formula (I) or a pharmaceutically acceptable salt thereof and one or more other therapeutic agents. Such combinations may be presented individually (wherein each active is in separate composition) or the actives are presented in a combined composition.

This invention provides a combination of a compound of Formula (I), or a pharmaceutically acceptable salt thereof, and one or more therapeutic agents for the treatment of an inflammatory disease and/or an autoimmune disease, for example, a TNF-a inhibitor; a non-selective COX-1/COX-2 inhibitor; a selective COX-2 inhibitor, such as celecoxib; agents including methotrexate, leflunomide, sulfasalazine, azathioprine, penicillamine, bucillamine, actarit, mizoribine, lobenzarit, hydroxychloroquine, d-penicillamine, aurothiomalate, auranofin, parenteral and/or oral gold, cyclophosphamide, a BAFF/APRIL inhibitor, CTLA-4-Ig, or a mimetic of CTLA-4-Ig; 5-lipoxygenase (5-LO) inhibitor, or a 5-lipoxygenase activating protein (FLAP) antagonist; a leukotriene modifier, including a leukotriene receptor antagonist, such as montelukast, zafirlukast, pranlukast; a phosphodiesterase type IV (PDE-IV) inhibitor, such as cilomilast (ariflo) or roflumilast; an antihistamine H1 receptor antagonist; anticholinergic agents such as muscarinic antagonists (ipratropium bromide and tiotropium bromide), as well as selective muscarinic M3 antagonists; β-adrenoceptor agonists such as salmeterol, formoterol, arformoterol, terbutaline, metaproterenol, albuterol and the like; a DP receptor antagonist, such as S-5751 and laropiprant; TP receptor antagonists such as seratrodast; neurokinin antagonists (1 NK2); VLA-4 antagonists; a corticosteroid, such as triamcinolone acetonide, budesonide, beclomethasone, fluticasone and mometasone; insulin-like growth factor type I (IGF-1) mimetic; kinase inhibitors including Janus Kinase inhibitors (e.g., JAK 1 and/or JAK2 and/or JAK 3 and/or TYK2), p38 MAPK, Syk or IKK2; rituximab; selective co-stimulation modulator such as abatacept; IL-1 inhibitor anakinra, IL-6 inhibitor tocilizumab, and IL12/IL-23 inhibitor ustekimumab; anti-IL17 antibody, anti-IL17R antibody, anti-IL21 antibody, or anti-IL22 antibody, S1P1 agonists including fingolimod; interferon beta 1; natalizumab; a mTOR inhibitor such as rapamycin, cyclosporine, tacrolimus; non-steroidal antiinflammatory agent (NSAID), including alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac, flufenamic acid, meclofenamic acid, mefenamic acid, niflumic acid, tolfenamic acid, diflunisal and flufenisal, isoxicam, piroxicam, sudoxicam, tenoxican, acetyl salicylic acid, apazone, bezpiperylon, feprazone, mofebutazone, oxyphenbutazone, phenylbutazone; fumaric acid derivative, BG-12; chemokine or chemokine receptor inhibitor, such as a CCR-1, CCR-2, CCR-3 and CCR-9 antagonist.

This invention further provides a combination of a compound of Formula (I), or a pharmaceutically acceptable salt thereof, and one or more therapeutic agents for the treatment of multiple myeloma, for example, Bortezomibdexamethasone, Bortezomibdexamethasone cyclophosphamide, Bortezomibdexamethasonelenalidomide, Lenalidomidedexamethasone, Melphalanprednisonethalidomide, Melphalanprednisonebortezomib, Melphalanprednisone lenalidomide, Lenalidomide-dexamethasone-clarithromycin and any of the above combinations plus agents used to treat bone disease in multiple myeloma including bisphosphonates, RANK-L inhibitors such as Denusomab and anabolic bone building drugs such as parathyroid hormone (PTH).

This invention also provides a combination of a compound of Formula (I), or a pharmaceutically acceptable salt thereof, and one or more therapeutic agents for the treatment of colon and/or rectal cancer, for example FOLFOX® (leucovorin [folinic acid], 5-Fluoruracil, and oxaliplatin), FOLFIRI® (leucovorin, 5-Fluoruracil, and irinotecan), CapeOX® (capecitabine and oxaliplatin), any of the above combinations plus either bevacizumab or cetuximab (but not both), 5-Fluoruracil and leucovorin, with or without bevacizumab, Capecitabine, with or without bevacizumab, FOLFOXIRI® (leucovorin, 5-Fluoruracil, oxaliplatin, and irinotecan), Irinotecan, with or without cetuximab, Cetuximab alone, and Panitumumab alone.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically acceptable excipient(s).

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional therapeutically active compounds.

As used herein, “pharmaceutically acceptable excipient” means a pharmaceutically acceptable material, composition, or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Compound Preparation

The compounds of Formula (I) may be obtained by using synthetic procedures illustrated in the Schemes below or by drawing on the knowledge of a skilled organic chemist. The reaction sequences provided in these Schemes are applicable for producing compounds of the invention having a variety of different X¹-X⁵, R¹, R³, R^{3a}, and R⁵ groups, as defined herein, employing appropriate precursors. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

The compounds of Formula (I) containing a benzofuran moiety may be prepared from commercially available phenol derivatives according to Schemes 1-4. Substituted aryl methyl amines of Formula (II) may be prepared from commercially available aryl nitrile starting materials according to Scheme 1. Substituted aryl acetic acids of Formula (III) may be prepared from commercially available aryl bromide starting materials according to Scheme 4. The compounds of Formula (I) containing a benzoxazole moiety may be prepared from commercially available phenol derivatives according to Schemes 5 and 6. The compounds of Formula (I) containing a benzimidazole moiety may be prepared from commercially available aniline derivatives according to Scheme 7. The compounds of Formula (I) containing a benzothiazole moiety may be prepared from commercially available aniline derivatives according to Scheme 8. The compounds of Formula (I) containing a quinoline moiety may be prepared from commercially available aniline derivatives according to Scheme 9. The compounds of Formula (I) containing a 1-oxo-1,2,3,4-tetrahydroisoquinolinyl moiety may be prepared from commercially available benzene derivatives according to Scheme 10.

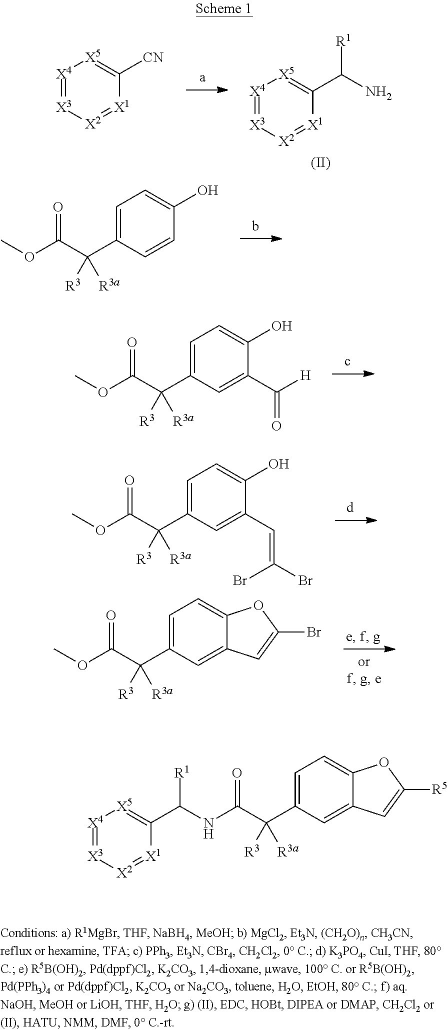

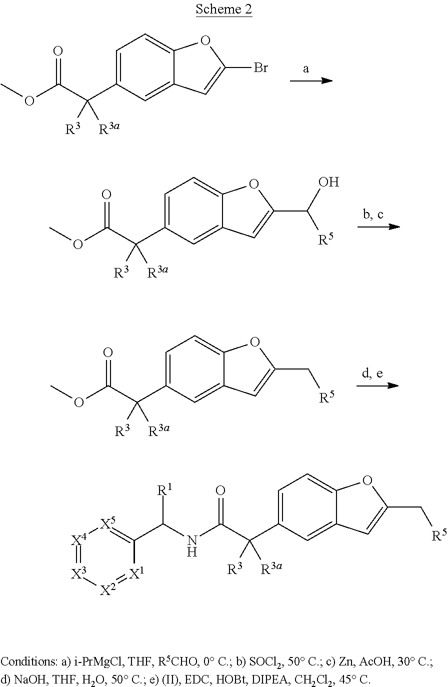

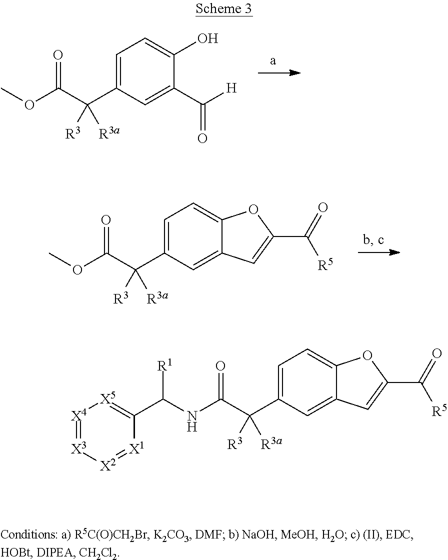

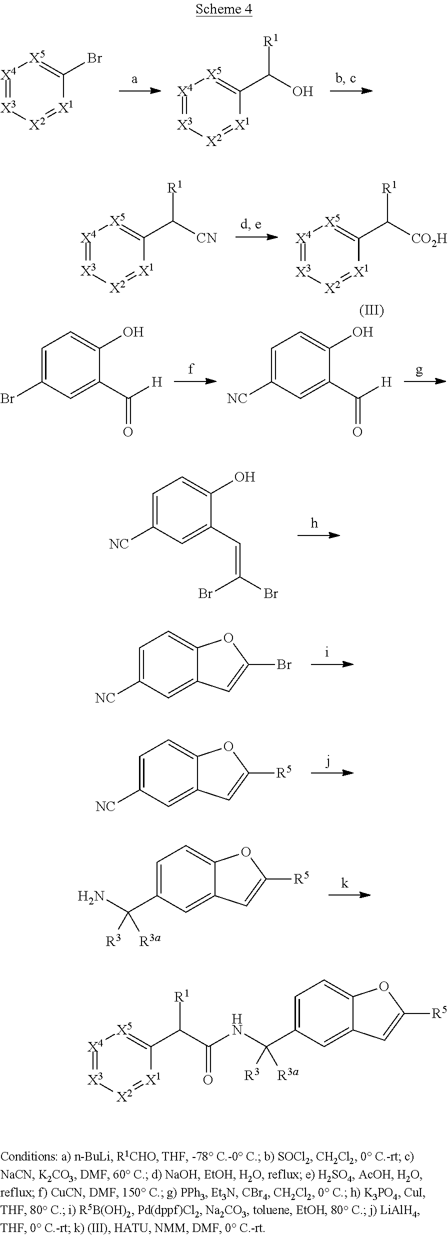

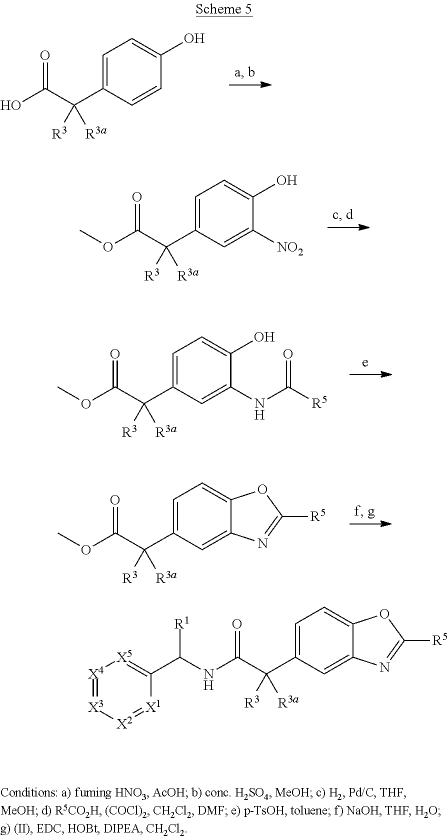

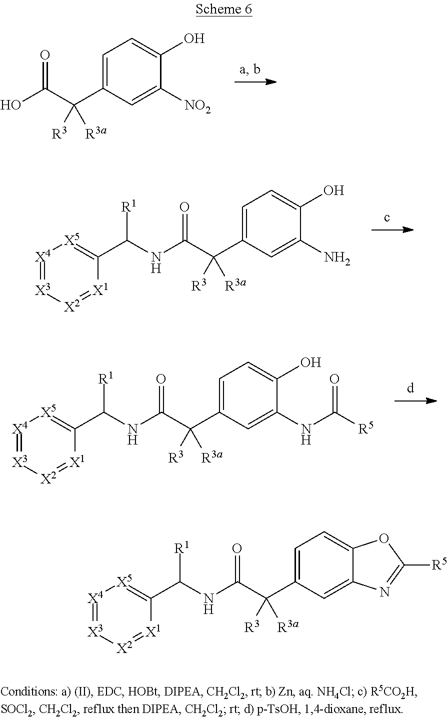

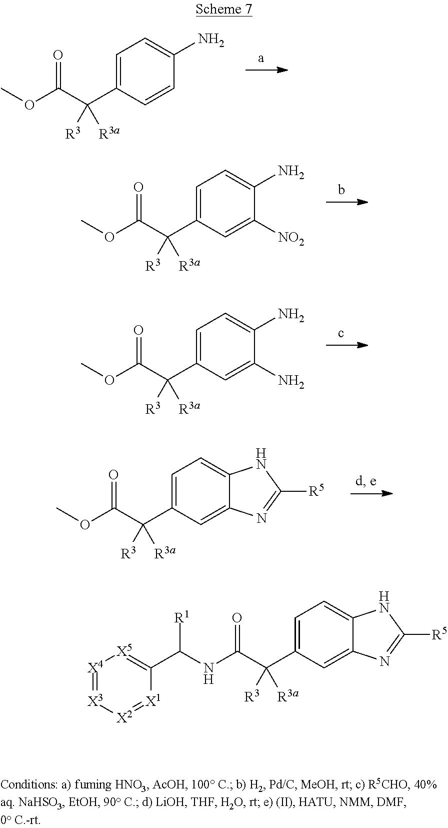

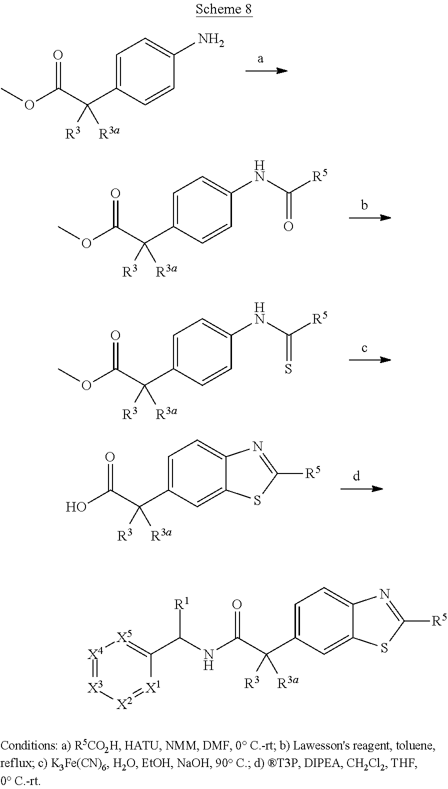

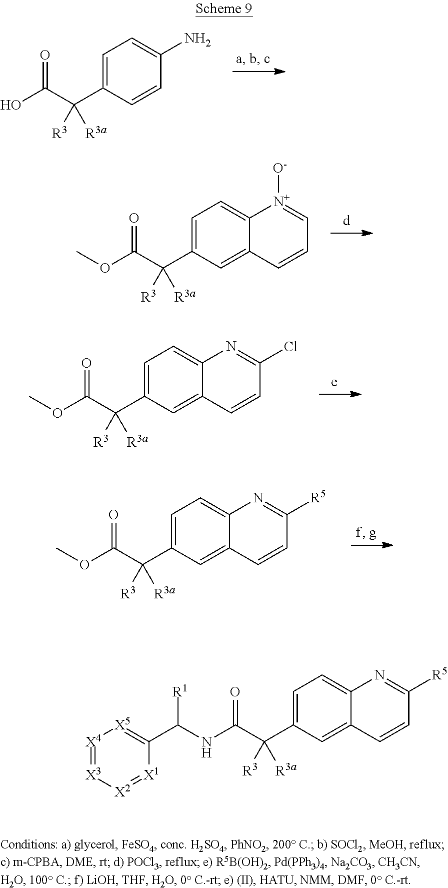

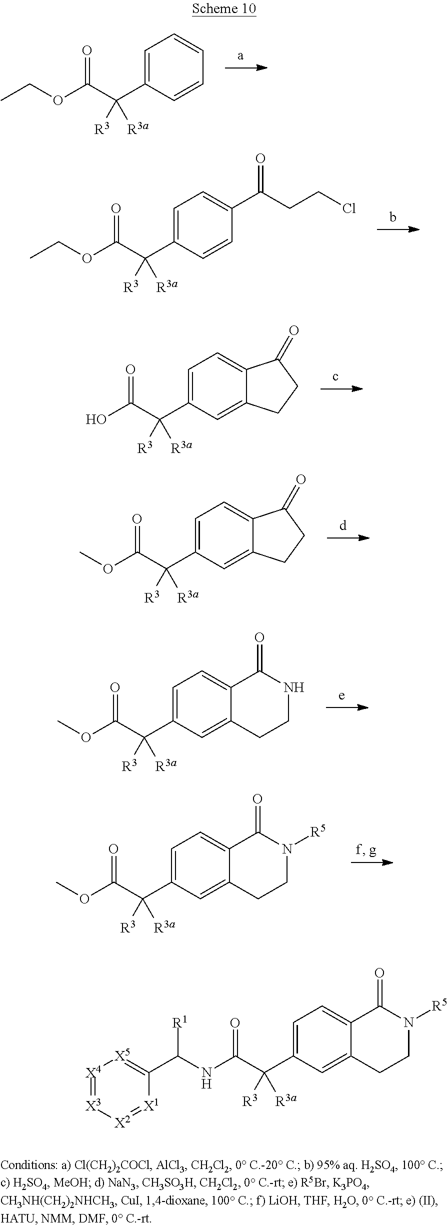

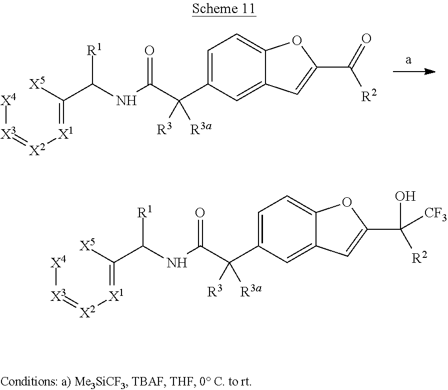

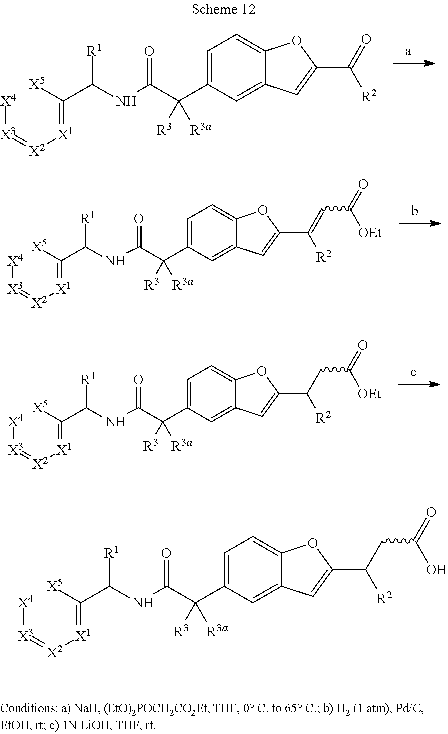

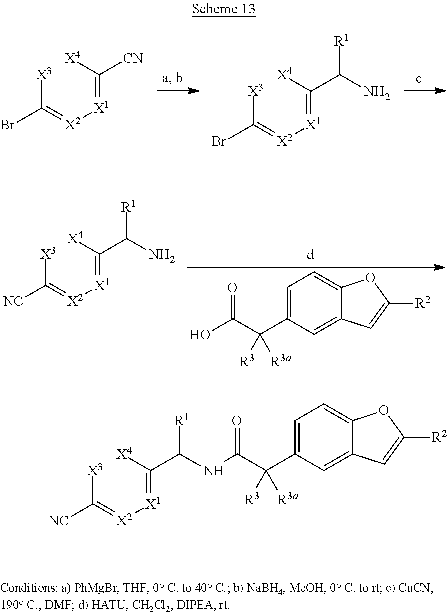

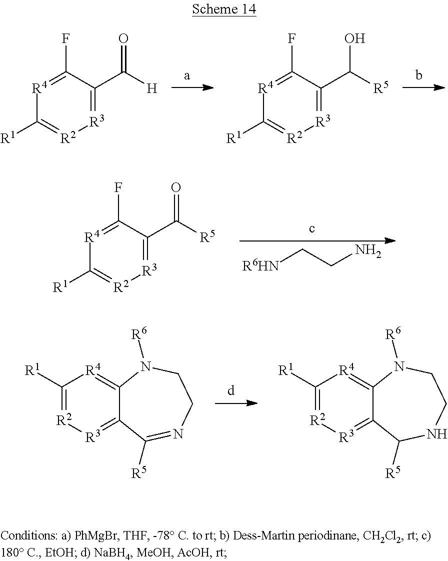

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Compounds names were generated using the software program ChemBioDraw Ultra V12.0 available from CambridgeSoft Corporation, 100 CambridgePark Drive, Cambridge, Mass. 02140 USA (http://www.cambridgesoft.com).

### Abbreviations

AcOH acetic acid
AIBN azobisisobutyronitrile
AlCl₃ aluminum trichloride
aq. aqueous
Ar argon gas
Br₂ bromine
CBr₄ carbon tetrabromide
CCl₄ carbon tetrachloride
CH₂Cl₂ dichloromethane
CH₃CN acetonitrile
CH₃I methyl iodide
(CH₂O)ₙ paraformaldehyde
CH₃SO₃H methanesulfonic acid
conc. Concentrated
Cs₂CO₃ cesium carbonate
CuBr copper(I) bromide
CuCN copper(I) cyanide
CuI copper(I) iodide
(COCl)₂ oxalyl chloride
DIPEA N,N-diisopropylethylamine
DMAP 4-(dimethylamino)pyridine
DME 1,2-dimethoxyethane
DMF N,N-dimethylformamide
DMSO dimethylsulfoxide
EtOAc ethyl acetate
EDC N-(3-dimethylaminopropyl)-N′-ethylcarbodiimide hydrochloride
Et₃N triethylamine
Et₂O diethyl ether
EtOH ethanol
FeSO₄ iron(II) sulfate
h hour(s)
H₂ hydrogen gas
HATU O-(7-azabenzotriazol-1-yl)-N,N,N′,N′-tetramethyluronium hexafluorophosphate
HBr hydrobromic acid
HCl hydrochloric acid
H₂O water
HNO₃ nitric acid
HOBt hydroxybenzotriazole
HPLC high-performance liquid chromatography
H₂SO₄ sulfuric acid
I₂ iodine
i-PrMgCl isopropylmagnesium chloride
K₂CO₃ potassium carbonate
K₃Fe(CN)₆ potassium ferricyanide
KOt-Bu potassium tert-butoxide
K₃PO₄ potassium phosphate tribasic
LCMS liquid chromatography mass spectrometry
LiAlH₄ lithium aluminum hydride
LiOH lithium hydroxide
m-CPBA meta-chloroperbenzoic acid
MeMgBr methyl magnesium bromide
MeOH methanol
Mg magnesium
MgCl₂ magnesium chloride
min minute(s)
MnO₂ manganese dioxide
N₂ nitrogen gas
NaB H₄ sodium borohydride
NaCN sodium cyanide
Na₂CO₃ sodium carbonate
NaH sodium hydride
NaHCO₃ sodium bicarbonate
NaHSO₃ sodium bisulfite
NaN₃ sodium azide
NaOH sodium hydroxide
Na₂SO₄ sodium sulfate
NBS N-Bromosuccinimide
n-BuLi n-butyllithium
NH₄Cl ammonium chloride
NMM N-methylmorpholine
PCC pyridinium chlorochromate
Pd/C palladium on carbon
Pd(dppf)Cl_{2 [}1,1′-bis(diphenylpho sphino)ferrocene]dichloropalladium(II)
Pd(PPh₃)₄ tetrakis(triphenylphosphine)palladium(0)
PhNO₂ nitrobenzene
POCl₃ phosphoryl chloride
PPh₃ triphenylphosphine
p-TsOH para-toluene sulfonic acid
R_{f} retention factor
rt room temperature
Rₜ retention time
SOCl₂ thionyl chloride
TFA trifluoroacetic acid
THF tetrahydrofuran
TLC thin layer chromatography
®T3P 2,4,6-tripropyl-1,3,5,2,4,6-trioxatriphosphorinane 2,4,6-trioxide
Zn zinc powder

### LCMS Conditions

LCMS-P1: Column:Waters Sunfire C18, 3.5 μm, 50×4.6 mm; Temperature: 50° C.; Mobile Phase: A: water (0.05% TFA) B: acetonitrile (0.05% TFA); Gradient: 5% B for 0.2 min, increase to 95% B within 1.2 min, 95% B for 1.6 min, return to 5% B within 0.01 min.; Flow Rate: 1.8 mL/min, Detection: PDA 190-400 nm

LCMS-G7: Column:XBridge C18, 3.6 μm, 50×4.6 mm; Temperature: 50° C.; Mobile Phase: A: water (0.1% formic acid) B: methanol; Gradient: 10% B for 0.1 min, increase to 95% B within 2.5 min, 95% B for 2.5 min, return to 10% B within 0.1 min, 10% B for 2 min. Flow Rate: 1.0 mL/min; Detection: PDA 190-400 nm

LCMS-G9: Column:XBridge C18, 3.6 μm, 50×4.6 mm; Temperature: 50° C.; Mobile Phase: A: water (0.1% ammonium acetate) B: methanol; Gradient: 10% B for 0.2 min, increase to 95% B within 5 min, 95% B for 2 min, return to 10% B within 0.1 min, 10% B for 2 min.; Flow Rate: 0.8 mL/min; Detection: PDA 190-400 nm

LCMS-G12: Column: Sunfire C18, 5 μm, 50×4.6 mm; Temperature: 50° C.; Mobile Phase: A: water (0.1% formic acid) B: methanol; Gradient: 30% B for 0.1 min, increase to 90% B within 4 min, 99% B for 4 min, return to 30% B within 0.1 min, 10% B for 2 min.; Flow Rate: 0.8 mL/min; Detection: PDA 190-400 nm

LCMS-G30: Column: Eclipse XDB C18, 5 μm, 250×4.6 mm; Temperature: 50° C.; Mobile Phase: A: water (0.05% TFA) B: acetonitrile (0.05% TFA); Gradient: 30% B for 0.2 min, increase to 95% B within 15 min, 95% B for 5 min, return to 30% B within 3 min 30% B for 5 min. Flow Rate: 0.8 mL/min; Detection: PDA 190-400 nm

LCMS-X: Column: Eclipse XDB C18, 5 μm, 150×4.6 mm; Temperature: 50° C.; Mobile Phase: A: water (0.1% formic acid) B: acetonitrile (0.1% formic acid); Gradient: 10% B for 0.1 min, increase to 90% B within 5 min, 100% within 2 min, 100% B for 4 min, return to 10% B within 0.01 min, 10% B for 1 min.; Flow Rate: 1.0 mL/min; Detection: PDA 190-400 nm

LCMS-T1: Column: Eclipse XDB C18, 5 μm, 150×4.6 mm; Temperature: 50° C. Mobile Phase: water (0.05% TFA) B: acetonitrile (0.05% TFA); Gradient: 5% B for 0.1 min, increase to 95% B within 7 min, 100% within 2 min, return to 5% B within 0.1 min, 5% B for 3 min.; Flow Rate: 1.0 mL/min; Detection: PDA 190-400 nm

LCMS-TFA: Column: Zorbax XDB C18, 3.5 μm, 50×4.6 mm; Temperature: 35° C.; Mobile Phase: water (0.05% TFA) B: acetonitrile (0.05% TFA); Gradient: 5% B for 0.1 min, increase to 100% B within 7 min, return to 5% B within 0.1 min, 5% B for 3 min.; Flow Rate: 1.0 mL/min; Detection: PDA 190-400 nm, (analyze at 220, 254, 280 nm)

LCMS-AMF: Column: Zorbax XDB C18, 3.5 μm, 50×4.6 mm; Temperature: 35° C.; Mobile Phase: water (10 mM ammonium formate) B: acetonitrile; Gradient: 5% B for 0.1 min, increase to 100% B within 7 min, 100%, return to 5% B within 0.1 min, 5% B for 3 min.; Flow Rate: 1.0 mL/min; Detection: PDA 190-400 nm, (analyze at 220, 254, 280 nm)

### Preparation 1

### 2-(4-hydroxy-3-nitrophenyl)acetic acid

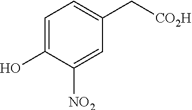 [SMILES: O=C(O)Cc1ccc(O)c([N+](=O)[O-])c1]

Concentrated nitric acid (57.5 mL, 592 mmol) was added drop-wise to a suspension of 2-(4-hydroxyphenyl)acetic acid (50 g, 328.9 mmol) in 500 mL acetic acid at 0° C. After the addition, the mixture was stirred at 0° C. for 1 h. The brown solution was then allowed to warm to rt, and a yellow precipitate was formed. The solid was collected and recrystallized from EtOAc/petroleum ether (1/3) to provide 2-(4-hydroxy-3-nitrophenyl)acetic acid as yellow solid (16.0 g, yield 24.7%). LC-MS: 198.1 [M+H]⁺; Rt=1.260 min. ¹H NMR (DMSO-d6, 400 MHz): δ 3.44 (s, 2H), 7.06 (d, J=8.4 Hz, 1H), 7.42 (dd, J₁=1.2 Hz, J₂=1.2 Hz, 1H), 7.79 (d, J=2.0 Hz, 1H), 10.84 (s, 1H), 12.42 (s, 1H).

### Preparation 2

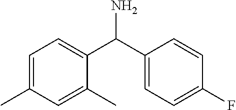 [SMILES: Cc1ccc(C(N)c2ccc(F)cc2)c(C)c1]

### (2,4-dimethylphenyl)(4-fluorophenyl)methanamine

4-Fluoro phenyl magnesium bromide (0.8 M in THF, 14.3 mL, 11.45 mmol) was added very slowly to a solution of 2,4-dimethylbenzonitrile (1.0 g, 7.63 mmol) in anhydrous THF (15 mL) at 0° C. After 10 min the reaction mixture was warmed to rt, and stirred for 1 h. The reaction mixture was then cooled to 0° C., and MeOH (5 mL) was added very slowly, followed by sodium borohydride (0.433 g, 11.45 mmol). The resulted mixture was refluxed for 4 h and then stirred at rt overnight. Crushed ice was then added to the reaction mixture. The reaction mixture was extracted with ethyl acetate (40 mL), and the combined extracts were washed with brine (15 mL), dried over Na₂SO₄ and concentrated under reduced pressure to obtained a crude product which was purified using silica gel chromatography using 20% EtOAc: Hexanes to yield (2,4-dimethylphenyl)(4-fluorophenyl)methanamine (0.500 g, 28.73%) as a light yellow semi solid. ¹H NMR (400 MHz, DMSO) δ 6.90-7.37 (m, 7H), 5.19 (s, 1H), 2.22 (s, 3H), 2.13 (s, 5H).

### Preparation 3

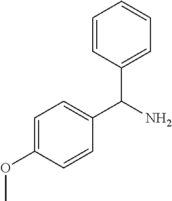 [SMILES: COc1ccc(C(N)c2ccccc2)cc1]

### (4-methoxyphenyl)(phenyl)methanamine

The title intermediate was synthesized from phenyl magnesium bromide and 4-methoxybenzonitrile following essentially the procedure of Example 1(c) (0.717 g, 89.52%) as a light brown oil. ¹H NMR (400 MHz, DMSO) δ 7.36-7.38 (d, 2H), 7.24-7.31 (m, 4H), 7.15-7.17 (d, 1H), 6.84-6.85 (d, 1H), 6.82 (s, 1H), 5.03 (s, 1H), 3.70 (s, 3H).

### Preparation 4

### 1-(2,6-dimethylpyridin-3-yl)-4-methylpentan-1-amine

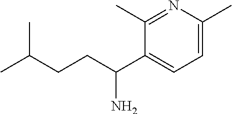

This intermediate was synthesized from 2,6-dimethylnicotinonitrile and isopentylmagnesium bromide essentially as described in example 2 (a) (500 mg, 6.2%). LC-MS: 207.1 [M+H]⁺; Rt: 1.26 min

### Preparation 5

### 4-methyl-1-(4-methyl-2-(trifluoromethyl)phenyl)pentan-1-amine

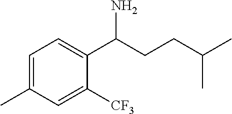 [SMILES: Cc1ccc(C(N)CCC(C)C)c(C(F)(F)F)c1]

This intermediate was synthesized from 4-methyl-2-(trifluoromethyl)benzonitrile and isopentylmagnesium bromide essentially as described in example 2 (a) (100 mg, Yield: 47.7%). LC-MSA024: 243.2 [M−NH2]+; Rt=1.33 min

### Preparation 6

### 1-(2,4-dichlorophenyl)-4-methylpentan-1-amine

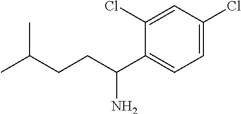

This intermediate was synthesized from 2,4-dichlorobenzonitrile and isopentylmagnesium bromide essentially as described in example 2 (a) (30 mg, Yield: 40%). LC-MS: 246 [M+H]+; Rt=1.35 min

### Preparation 7

### 1-(2,4-dichlorophenyl)-4-methylpentan-1-amine

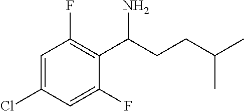 [SMILES: CC(C)CCC(N)c1c(F)cc(Cl)cc1F]

This compound was synthesized from 4-chloro-2,6-difluorobenzonitrile and isopentylmagnesium bromide essentially as described in example 2 (a) (25 mg, Yield: 27%). LC-MSA024: 237.1 [M−NH2]+; Rt=1.30 min.

### Preparation 8

### 6-methylpyrimidine-4-carboxylic acid

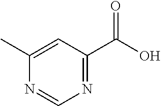

### a) methyl 6-methylpyrimidine-4-carboxylate

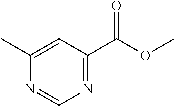

A 500 mL round bottom flask was charged with 400 mL H₂O and KMnO₄ (22.1 g, 140 mmol). 4,6-Dimethylpyrimidine was then added (35 mmole) and the mixture was refluxed for 20 h. After cooling to rt, the mixture was filtered through a pad of Celite. The filtrate was acidified to pH ˜3. All the solvent was concentrated under reduced pressure. MeOH saturated with HCl (300 mL) was then added to the resulting residue and the mixture was then refluxed for 15 h, and concentrated under reduced pressure. The resulting oil was then dissolved in CH₂Cl₂ (150 mL). The organic phase was separated, washed with saturated NaHCO₃, dried over MgSO₄, and concentrated under reduced pressure. The resulting residue was purified by column chromatography (SiO₂, 10 percent ether-methylene chloride) to yield methyl 6-methylpyrimidine-4-carboxylate (1.12 g, 21.5%). LCMSA036: 153.2 [M+H]⁺; Rt=1.14 min; Purity: 34.4% (254 nm).

### b) 6-methylpyrimidine-4-carboxylic acid

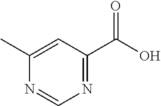

LiOH.H₂O (262 mg, 10.9 mmol) was added to a solution of methyl 6-methylpyrimidine-4-carboxylate1.12 g, 7.30 mmol) in 15 mL of MeOH and 3 mL of water. The mixture was stirred at rt for 2 h. The mixture was then acidified with 0.5N HCl solution to pH 3-4, and all solvent was concentrated under reduced pressure to obtain 6-methylpyrimidine-4-carboxylic acid (800 mg, yield: 80%). LC-MS036: 139.1[M+H]⁺; Rt=0.93 min.

### Preparation 9

### (4-methyl-2-(trifluoromethyl)phenyl)(phenyl)methanamine

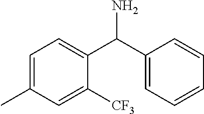 [SMILES: Cc1ccc(C(N)c2ccccc2)c(C(F)(F)F)c1]

### a) (4-methyl-2-(trifluoromethyl)phenyl)(phenyl)methanimine

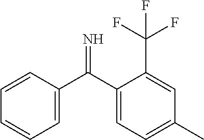 [SMILES: Cc1ccc(C(=N)c2ccccc2)c(C(F)(F)F)c1]

Phenylmagnesium bromide (1M in THF, 4.05 mL, 4.05 mmol) was added to a solution of 4-methyl-2-(trifluoromethyl)benzonitrile (250 mg, 1.35 mmol) in THF (10 mL) and stirred at 70° C. overnight. The reaction mixture was then diluted with EtOAc (30 mL), washed with brine (30 mL), dried over Na₂SO₄, and concentrated under reduced pressure. The resulting residue was purified by Pre-TLC (Petroleum ether: EtOAc=5:1) to afford (4-methyl-2-(trifluoromethyl)phenyl)(phenyl)methanimine (110 mg, yield: 31%) as a yellow oil. LCMSA027: 264[MH]⁺; Rt: 0.94 min.

### b) (4-methyl-2-(trifluoromethyl)phenyl)(phenyl)methanamine

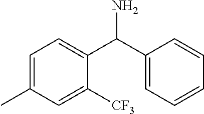 [SMILES: Cc1ccc(C(N)c2ccccc2)c(C(F)(F)F)c1]

A mixture of (4-methyl-2-(trifluoromethyl)phenyl)(phenyl)methanimine (110 mg, 0.41 mmol), NH₃H₂O (1 mL) and Raney-Ni (485 mg, 8.36 mmol) in MeOH (20 mL) was stirred under H₂ atmosphere (1 atm) at rt overnight. The reaction mixture was then filtered through a pad of Celite, and concentrated under reduced pressure to afford (4-methyl-2-(trifluoromethyl)phenyl)(phenyl)methanamine (90 mg, yield: 81%) as a yellow oil. LCMSA020: 249[M−NH₂]⁺; Rt: 1.75 min

### Preparation 10

### 2-(amino(phenyl)methyl)-N,N,5-trimethylaniline

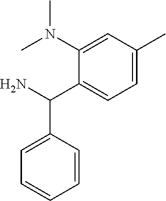 [SMILES: Cc1ccc(C(N)c2ccccc2)c(N(C)C)c1]

### a) 2-(dimethylamino)-4-methylbenzonitrile

A mixture of 2-amino-4-methylbenzonitrile (400 mg, 3 mmol), HCHO (37% aq, 4.91 g, 60 mmol), AcOH (0.5 mL) and NaCNBH3 (378 mg, 9 mmol) in CH₃CN (20 mL) was stirred at rt overnight. The mixture was then concentrated under reduced pressure, diluted with EtOAc (30 mL), washed with brine (40 mL), dried over sodium sulfate, filtered, concentrated under reduced pressure, and purified by Pre-TLC(Petroleum ether: EtOAc=7:1) to afford 2-(dimethylamino)-4-methylbenzonitrile (230 mg, 47%) as yellow oil. LC-MSA027: m/z 161 [M+H]+; Rt=1.11 min Purity 93% (214 nm).

### b) 2-(amino(phenyl)methyl)-N,N,5-trimethylaniline

This intermediate was synthesized from 2-(dimethylamino)-4-methylbenzonitrile and phenylmagnesium bromide essentially as described in preparation 9 (150 mg, crude) as a yellow oil. LCMSA036: 224[M−NH₂]⁺; Rt: 1.24 min

### Preparation 11

### 2-(amino(phenyl)methyl)-5-methylphenol

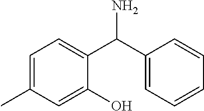 [SMILES: Cc1ccc(C(N)c2ccccc2)c(O)c1]

This compound was synthesized from 2-hydroxy-4-methylbenzonitrile and phenylmagnesium bromide essentially as described in example 2 (a) (324 mg, yield: 50%, 2 steps). LC-MS (036): 197.1 [M−NH₂]⁺; Rt=1.11 min.

### Preparation 12

### 1-(4-fluorophenyl)-6-methyl-1,2,3,4-tetrahydroisoquinoline

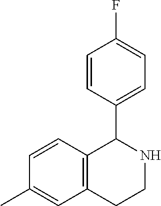 [SMILES: Cc1ccc2c(c1)CCNC2c1ccc(F)cc1]

### a) 2-(m-tolyl)ethanamine

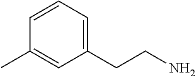

This intermediate was synthesized from 2-(m-tolyl)acetonitrile essentially as described in Preparation 9 (b) (4.6 g, yield: 90%). LC-MS (020): 136.2 [M+H]⁺; Rt: 1.217 min, Purity 83% (254).

### b) 1-(4-fluorophenyl)-6-methyl-1,2,3,4-tetrahydroisoquinoline

A solution of 2-(m-tolyl)ethanamine (1.35 g, 10 mmol) and 4-fluorobenzaldehyde (1.24 g, 10 mmol) in 20 mL of EtOH was refluxed for 2 h and then EtOH was removed under reduced pressure. Trifluoromethanesulfonic acid (20 mL) was added to the resulting residue and the solution was stirred at 60° C. overnight. After cooling to rt, the mixture was poured over ice-water and basified with 1 N NaOH to pH 11. The mixture was extracted with CH₂Cl₂ (30 mL×3). The combined extracts were washed with brine (10 mL), dried over Na₂SO₄, and concentrated under reduced pressure. The resulting residue was purified by flash column (petroleum ether/EtOAc 5/1) to give 1-(4-fluorophenyl)-6-methyl-1,2,3,4-tetrahydroisoquinoline (935 mg, yield: 32%).

### Preparation 13

### 2-(2-(1-hydroxy-1-(pyridin-4-yl)ethyl)benzofuran-5-yl)acetic acid

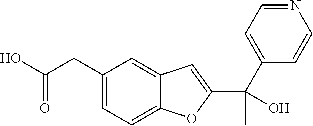 [SMILES: CC(O)(c1ccncc1)c1cc2cc(CC(=O)O)ccc2o1]

### a) 1-(pyridin-4-yl)-3-(triisopropylsilyl)prop-2-yn-1-ol

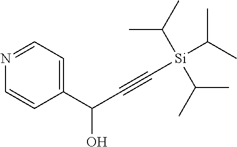 [SMILES: CC(C)[Si](C#CC(O)c1ccncc1)(C(C)C)C(C)C]

n-BuLi (2.5M, 98 mL) was added to a solution of ethynyltriisopropylsilane (49 g, 268.8 mmol) in THF (500 mL) at 0° C. under N₂ atmosphere. The mixture was stirred at 0° C. for 1 h, then isonicotinaldehyde (24 g, 224 mmol) was added drop-wise, and the resulting reaction mixture was stirred at 0° C. for 1 h. A saturated NH₄Cl aqueous solution (200 mL) was then added, and the mixture was extracted with EtOAc (300 mL×3). The combined extracts were washed with brine (500 mL×3), dried over Na₂SO₄, and concentrated under reduced pressure to yield 1-(pyridin-4-yl)-3-(triisopropylsilyl)prop-2-yn-1-ol (62 g, crude) as a yellow oil. LCMSA027: 290[MH]⁺; Rt: 1.45 min

### b) 1-(pyridin-4-yl)-3-(triisopropylsilyl)prop-2-yn-1-one

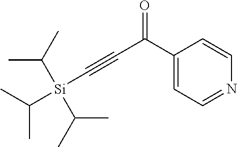 [SMILES: CC(C)[Si](C#CC(=O)c1ccncc1)(C(C)C)C(C)C]

MnO₂ (45 g, 519 mmol) was added portion-wise to a solution of 1-(pyridin-4-yl)-3-(triisopropylsilyl)prop-2-yn-1-ol (30 g, 103.8 mmol) in CH₂Cl₂ (500 mL) and the reaction mixture was refluxed for 2 h. The reaction was then filtered through of a pad of Celite and the filtrate was concentrated under reduced pressure to afford 1-(pyridin-4-yl)-3-(triisopropylsilyl)prop-2-yn-1-one (27.8 g, yield: 93%) as a yellow oil. LCMSA020: 288.2[MH]⁺; Rt: 2.06 min.

### c) 2-(pyridin-4-yl)-4-(triisopropylsilyl)but-3-yn-2-ol

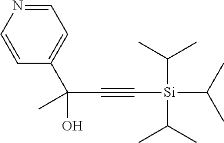 [SMILES: CC(C)[Si](C#CC(C)(O)c1ccncc1)(C(C)C)C(C)C]

MeMgBr (3M, 27.87 mL) was addd to a solution of 1-(pyridin-4-yl)-3-(triisopropylsilyl)prop-2-yn-1-one (8 g, 27.87 mmol) in THF (100 mL) at 0° C. under nitrogen atmosphere. The reaction mixture was stirred at 0° C. for 2 h, and then quenched with 100 mL NH₄Cl (aq), and extracted with EtOAc (100 mL×3). The combines extracts were washed with brine (50 mL), dried over sodium sulfate, and concentrated under reduced pressure to yield 2-(pyridin-4-yl)-4-(triisopropylsilyl)but-3-yn-2-ol (5.1 g, crude) as a yellow solid. LCMSA027: 304.0[MH]⁺; Rt: 1.16 min.

### d) 2-(pyridin-4-yl)but-3-yn-2-ol

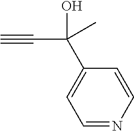

TBAF (35.36 mmol, 35.36 mL, 1N in THF) was added to a solution of 2-(pyridin-4-yl)-4-(triisopropylsilyl)but-3-yn-2-ol (8.93 g, 29.47 mmol) in THF (30 mL). The reaction mixture was stirred at rt for 2 h. A saturated NH₄Cl solution (100 mL) was then added, and the mixture was extracted with EtOAc (100 mL×3). The combined extracts were washed with brine (50 mL×3), dried over sodium sulfate, and concentrated under reduced pressure. Thre resulting residue was purified by silica gel column (Petroleum ether: EtOAc=10:1-5:1) to give 2-(pyridin-4-yl)but-3-yn-2-01 (3.6 g, 83.7%) as a yellow oil. LCMSA027: 148.1 [M+H]⁺; Rt: 0.46 min.

### e) methyl 2-(2-(1-hydroxy-1-(pyridin-4-yl)ethyl)benzofuran-5-yl)acetate

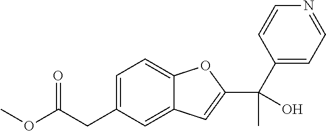 [SMILES: COC(=O)Cc1ccc2oc(C(C)(O)c3ccncc3)cc2c1]

A mixture of 2-(pyridin-4-yl)but-3-yn-2-ol (3.6 g, 24.48 mmol), methyl 2-(4-hydroxy-3-iodophenyl)acetate (4.75 g, 16.32 mmol), Pd(PPh₃)₂Cl₂(1.13 g, 1.632 mmol) and CuI (402 mg, 2.12 mmol) in Et₃N (20 mL) and DMF (20 mL) was stirred under N₂ atmosphere at 100° C. overnight. The reaction was then filtered through of a pad of Celite, and the filtrate was concentrated under reduced pressue. Water (200 mL) was added, and the resulting mixture was extracted with EtOAc (50 mL×3). The combined extracts were washed with brine (100 mL), dried over Na₂SO₄, and concentrated under reduced pressure. The resulting residue was purified by silica gel column (Petroleum ether:EtOAc=5:1-3:1) to afford methyl 2-(2-(1-hydroxy-1-(pyridin-4-yl)ethyl)benzofuran-5-yl)acetate (4.8 g, 94%) as a yellow oil. LCMSA027: 311.9[M+H]⁺; Rt: 0.89 min.

### f) 2-(2-(1-hydroxy-1-(pyridin-4-yl)ethyl)benzofuran-5-yl)acetic acid

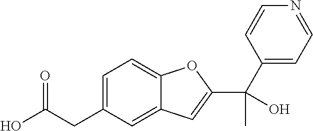 [SMILES: CC(O)(c1ccncc1)c1cc2cc(CC(=O)O)ccc2o1]

A solution of LiOH (740 mg, 30.86 mmol) in water (5 mL) was added to a mixture of methyl 2-(2-(1-hydroxy-1-(pyridin-4-yl)ethyl)benzofuran-5-yl)acetate (4.8 g, 15.43 mmol) in MeOH (20 mL), and the reaction mixture was stirred at 40° C. for 30 min. The reaction mixture was then concentrated under reduced pressure to remove the MeOH, and water (50 mL) was added. The pH of the reaction mixture was adjusted to ˜5-6 with diluted HCl. Then the mixture was extracted with EtOAc (50 mL×3). The combined extracts were washed with brine, dried over sodium sulfate, and concentrated under reduced pressure to yield 2-(2-(1-hydroxy-1-(pyridin-4-yl)ethyl)benzofuran-5-yl)acetic acid (4 g, yield: 87%) as a white solid. LCMSA020: 298.1[M+H]⁺; Rt: 1.18 min. ¹H NMR (MeOD, 400 MHz): δ 12.27 (s, 1H), 7.48-7.40 (m, 5H), 7.15 (m, 1H), 6.81 (s, 1H), 6.37 (s, 1H), 3.63 (s, 2H), 1.84 (s, 3H).

### Preparation 14

### 2-(2-(1-(3-fluoropyridin-4-yl)-1-hydroxyethyl)benzofuran-5-yl)acetic acid

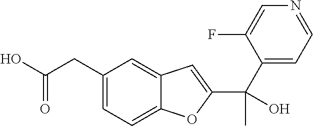 [SMILES: CC(O)(c1cc2cc(CC(=O)O)ccc2o1)c1ccncc1F]

The title intermediate was synthesized from 3-fluoroisonicotinaldehydefollowing essentially the procedure described in Preparation 13. LCMSA038: 316[M+H]⁺; Rt: 1.44 min. ¹HNMR (MeOD, 400 MHz): δ 8.50 (s, 2H), 7.89 (s, 1H), 7.51 (s, 1H), 7.35 (d, J=8.4 Hz, 1H), 7.20 (d, J=8.4 Hz, 1H), 6.74 (s, 1H), 3.70 (s, 2H), 2.03 (s, 3H)

### Preparation 15

### 6-methyl-1-phenyl-1,2,3,4-tetrahydroisoquinoline and 8-methyl-1-phenyl-1,2,3,4-tetrahydroisoquinoline

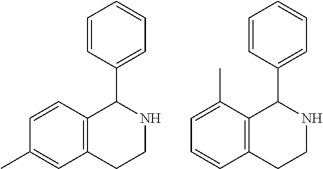

The title intermediates were synthesized from f 2-(m-tolyl)acetonitrile and benzaldehyde following essentially the procedure described in Preparation 12 and they were obtained in the following amounts:

6-methyl-1-phenyl-1,2,3,4-tetrahydroisoquinoline: 800 mg, yield: 32%. LC-MS (020): 224.2 [M+H]⁺; Rt: 1.682 min. ¹H NMR (CDCl₃, 400 MHz): δ 7.33-7.25 (m, 5H), 6.96 (s, 1H), 6.85 (d, J=7.6 Hz, 1H), 6.63 (d, J=7.6 Hz, 1H), 5.07 (s, 1H), 3.29-3.24 (m, 1H), 3.11-2.99 (m, 2H), 2.81-2.75 (m, 1H), 1.93 (s, 3H).

8-methyl-1-phenyl-1,2,3,4-tetrahydroisoquinoline: 150 mg, yield 6%. LC-MS (020): 224.1 [M+H]⁺; Rt: 1.646 min.

### Preparation 16

### 4-(2,4-dimethylphenyl)tetrahydro-2H-pyran-4-amine

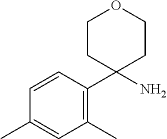

### a) 4-(2,4-dimethylphenyl)tetrahydro-2H-pyran-4-ol

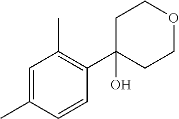

A solution of 1-bromo-2,4-dimethylbenzene (5000 mg, 27.2 mmol) in THF (25 mL) was cooled to −78° C. under N₂ protection and n-BuLi (13 mL, 32.6 mmol) was added drop-wise to the mixture. After addition, it was stirred for 30 min under this temperature. A solution of tetrahydropyran-4-one (2700 mg, 27.2 mmol) dissolved in THF (5 mL) was added dropwise to the mixture. The mixture was allowed to warm up to rt and stirred overnight. NH₄Cl (10 mL) was added to reaction mixture, and the mixture was extracted with ethyl acetate (20 mL×3). The combined organic layers were washed with brine (15 mL×3), dried over Na₂SO₄, and concentrated under reduced pressure. The resulting oil was purified by column chromatography on silica gel (petroleum ether: EtOAc=5:1) to give 4-(2,4-dimethylphenyl)tetrahydro-2H-pyran-4-ol as a white solid (4000 mg, yield 90%). LC-MSA036: 188.70 [M−OH]⁺; Rt=1.576 min, purity 90% (214 nm).

### b) 2-chloro-N-(4-(2,4-dimethylphenyl)tetrahydro-2H-pyran-4-yl)acetamide

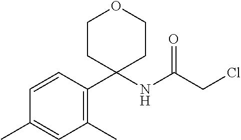 [SMILES: Cc1ccc(C2(NC(=O)CCl)CCOCC2)c(C)c1]

A solution of 4-(2,4-dimethylphenyl)tetrahydro-2H-pyran-4-ol (2000 mg, 9.7 mmol) dissolved in 2-chloroacetonitrile (6.7 mL), then it was cooled to 0° C. A solution of conc. H₂SO₄ (4 drops) in AcOH (13 mL) was added drop-wise to the mixture. After addition, the mixture was stirred overnight at rt. It was basified with 5N NaOH to pH ˜8, and extracted with EtOAc (15 mL×3). The combined extracts were dried over Na₂SO₄, and concentrated under reduced pressure. The resulting residue was purified by column chromatography on silica gel by EtOAc: petroleum ether=3:1 to afford 2-chloro-N-(4-(2,4-dimethylphenyl)tetrahydro-2H-pyran-4-yl)acetamide as a white solid (230 mg, yield 8.4%). LCMSA036: 304.70 [M+Na]⁺; Rt=1.577 min.

### c) 4-(2,4-dimethylphenyl)tetrahydro-2H-pyran-4-amine

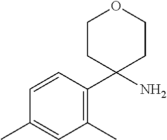

A solution of 2-chloro-N-(4-(2,4-dimethylphenyl)tetrahydro-2H-pyran-4-yl)acetamide (700 mg, 2.49 mmol) and thiourea (227 mg, 2.99 mmol) in AcOH (1.12 mL) and EtOH (7 mL) was refluxed overnight. Water (5 mL) was then added to the mixture, followed by 5N NaOH until the pH of the solution was ˜8. Then the mixture was extracted with EtOAc (10 mL×3). The combined extracts were dried over Na₂SO₄, and concentrated under reduced pressure. The resulting residue was purified by column chromatography on silica gel by CH₂Cl₂: MeOH=10:1 to afford the title intermediate as a white solid (150 mg, yield 29.4%). LCMSA044: 188[M−NH2]⁺; Rt=1.63 min.

### Preparation 17

### (2-methoxyphenyl)(phenyl)methanamine

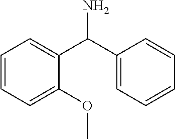 [SMILES: COc1ccccc1C(N)c1ccccc1]

This intermediate was synthesized from 2-methoxybenzonitrile and phenylmagnesium bromide essentially as described in example 2 (a). LC-MS of compound: 197.0 [M−NH2]⁺; Rt=1.17 min, Purity 60% (214).

### Preparation 18

### phenyl(o-tolyl)methanamine

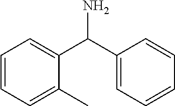

This intermediate was synthesized from 2-methylbenzonitrile and phenylmagnesium chloride essentially as described in example 2 (a). (360 mg, 43%). LCMS-P1: 181 [M−NH₂]⁺; Rₜ: 1.604 min. ¹H NMR (500 MHz, CDCl₃) δ ppm 7.59-7.18 (m, 9H), 5.41 (s, 1H), 2.30 (s, 3H), 1.84 (s, 2H).

### Preparation 19

### 2-bromo-1-(4-chlorophenyl)ethanone

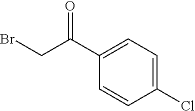 [SMILES: O=C(CBr)c1ccc(Cl)cc1]

Br₂ (23 g, 146 mmol) was added drop-wise to a solution of 1-(4-chlorophenyl)ethanone (20 g, 139 mmol) in HOAc/HBr (160 mL). After the addition, the mixture was stirred at rt for 2 h. Then the mixture was poured into the ice-water, and extracted with EtOAc (3×150 mL). The combined extracts were dried over Na₂SO₄, and concentrated under reduced pressure. The resulting residue was recrystallized from petroleum ether (100 mL) to get 2-bromo-1-(4-chlorophenyl)ethanone (9.8 g, 32.6%). LC-MS (036): 232.9 [M+H]⁺; Rt: 1.71 min.

### Preparation 20

### 2-(2-(1-hydroxy-1-(pyridin-3-yl)ethyl)benzofuran-5-yl)acetic acid

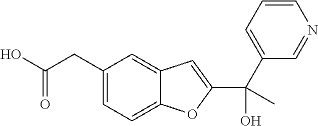 [SMILES: CC(O)(c1cccnc1)c1cc2cc(CC(=O)O)ccc2o1]

The title intermediate was synthesized from nicotinaldehyde following essentially the procedure described in Preparation 13. LCMSA036: 298.7[M+H]⁺; Rt: 1.07 min.

### Preparation 21

### ethyl 2-(2-(1-hydroxy-1-(2-hydroxypyridin-4-yl)ethyl)furo[3,2-b]pyridin-5-yl)acetate

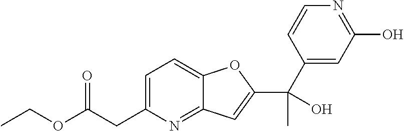 [SMILES: CCOC(=O)Cc1ccc2oc(C(C)(O)c3ccnc(O)c3)cc2n1]

The title intermediate was synthesized from 2-hydroxyisonicotinaldehyde following essentially the procedure described in Preparation 13 steps (a) through (e). LC-MS (036): 343.2; Rt=1.22 min

### Preparation 22

### 1-(3,5-dimethylisoxazol-4-yl)prop-2-yn-1-ol

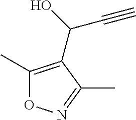

### a) 1-(3,5-dimethylisoxazol-4-yl)-3-(triisopropylsilyl)prop-2-yn-1-ol

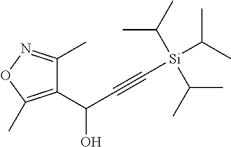 [SMILES: Cc1noc(C)c1C(O)C#C[Si](C(C)C)(C(C)C)C(C)C]

n-BuLi (48 mmol, 19 mL, 2.5 N in THF) was added to a solution of ethynyltriisopropylsilane (8 g, 44 mmol) in THF (30 mL) at 0° C. After stirring at 0° C. for 30 min, a solution of 3,5-dimethylisoxazole-4-carbaldehyde (2, 5 g, 40 mmol) in THF (20 mL) was added. The reactio mixture was further stirred for 2 h at 0° C., quenched with 2N HCl (24 mL), and extracted with EtOAc. The combined extracts were concentrated under reduced pressure, and the resulting residue was purified by flash column (petroleum ether/EtOAc=5/1) to give 1-(3,5-dimethylisoxazol-4-yl)-3-(triisopropylsilyl)prop-2-yn-1-ol (11 g, yield: 92%) as an oil. LCMSA039: 308[M+H]+; Rt: 1.98 min

### b) 1-(3,5-dimethylisoxazol-4-yl)prop-2-yn-1-ol

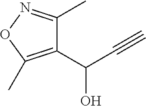

TBAF (19.7 mmol, 19.7 mL, 1N in THF) was added to a solution of 1-(3,5-dimethylisoxazol-4-yl)-3-(triisopropylsilyl)prop-2-yn-1-ol (5.5 g, 179 mmol) in THF (50 mL) at 0° C. After stirring at 0° C. for 2 h, the reaction mixture was extracted with EtOAc. The combined extracts were dried over Na₂SO₄, and concentrated under reduced pressure. The resulting resiude was purified by flash column (petroleum ether/EtOAc=2/1) to give 1-(3,5-dimethylisoxazol-4-yl)prop-2-yn-1-ol (2.45 g, yield: 90%) as an oil. LCMSA036: 152.1[M+H]+; Rt: 1.21 min.

### Preparations 23 and 24

### 6-chloro-1-phenyl-1,2,3,4-tetrahydroisoquinoline and 8-chloro-1-phenyl-1,2,3,4-tetrahydroisoquinoline

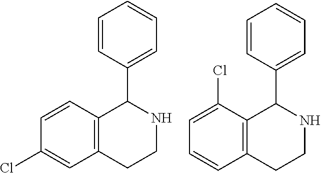

### a) N-benzylidene-2-(3-chlorophenyl)ethanamine

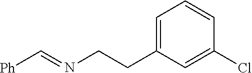 [SMILES: Clc1cccc(CC/N=C/c2ccccc2)c1]

A solution of 2-(3-chlorophenyl)ethanamine (1.55 g, 10 mmol) and benzaldehyde (1.06 g, 10 mmol) in 20 mL of EtOH was refluxed for 2 h. EtOH was then removed under reduced pressure and the residue was put under vacuum to remove all the volatile solvent and it was carried through without further purification (2.5 g, yield 100%).

### b) 6-chloro-1-phenyl-1,2,3,4-tetrahydroisoquinoline and 8-chloro-1-phenyl-1,2,3,4-tetrahydroisoquinoline

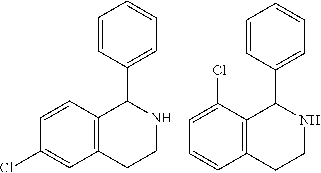

A solution of N-benzylidene-2-(3-chlorophenyl)ethanamine (2.4 g, 10 mmol) in 25 mL of trifluoromethanesulfonic acid was stirred at 90° C. overnight. After cooling to rt, the mixture was poured into ice-water and neutralized with 1 N NaOH to pH ˜8. The mixture was extracted with CH₂Cl₂ (30 mL×3). The combined extracts were washed with brine (10 mL), dried over Na₂SO₄, and concentrated under reduced pressure. The resulting residue was purified by flash column (petroleum ether/EtOAc=4/1) to give 800 mg of 6-chloro-1-phenyl-1,2,3,4-tetrahydroisoquinoline and 8-chloro-1-phenyl-1,2,3,4-tetrahydroisoquinoline as a mixture (in a 2:1 ratio). LC-MS (038): 244.1 [M+H]⁺; Rt: 1.32 min for 8-chloro-1-phenyl-1,2,3,4-tetrahydroisoquinoline and 1.35 min for 6-chloro-1-phenyl-1,2,3,4-tetrahydroisoquinoline.

### Preparation 25

### 6-methoxy-1-phenyl-1,2,3,4-tetrahydroisoquinoline

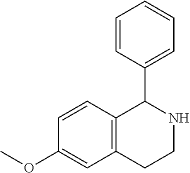 [SMILES: COc1ccc2c(c1)CCNC2c1ccccc1]

### a) N-benzylidene-2-(3-methoxyphenyl)ethanamine

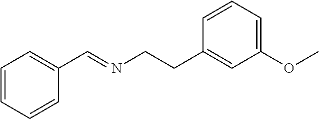 [SMILES: COc1cccc(CC/N=C/c2ccccc2)c1]

This intermediate was synthesized from 2-(3-methoxyphenyl)ethanamine and benzaldehyde essentially as described in preparations 23 and 24 (a) (8 g, crude).

### b) 6-methoxy-1-phenyl-1,2,3,4-tetrahydroisoquinoline

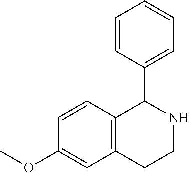 [SMILES: COc1ccc2c(c1)CCNC2c1ccccc1]

A solution of N-benzylidene-2-(3-methoxyphenyl)ethanamine (2 g, 8.4 mmol) in TFA (8 ml) was irradiated at 150 W in a microwave oven at 90° C. for 10 min. The mixture was then poured into 10 ml ice-water, basified to pH 8-9 with 2N NaOH, and extracted with CH₂Cl₂ (50 ml). The combined extracts were washed with 20 ml brine, dried over sodium sulfate, and concentrated under reduced pressure. The resulting residue was recrystallized from MeOH and petroleum ether to afford 6-methoxy-1-phenyl-1,2,3,4-tetrahydroisoquinoline (0.68 g, yield: 34%) as white solid. LCMSA (044): 240 [M+H]⁺; Rt: 1.75 min.

### Preparation 26

(5)-2-(2-((3,5-dimethylisoxazol-4-yl) (hydroxy)methyl)benzofuran-5-yl)acetic acid

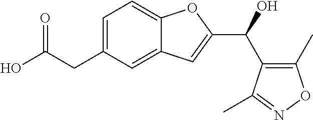 [SMILES: Cc1noc(C)c1[C@H](O)c1cc2cc(CC(=O)O)ccc2o1]

### a) (S)-1-(3,5-dimethylisoxazol-4-yl)-3-(triisopropylsilyl)prop-2-yn-1-ol

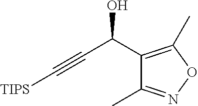

(Triisopropylsilyl)acetylene (219 g, 1.2 mol) was added to a diethylzinc solution (1.1 L, 1.02 Kg, 1.0M in toluene, 1.1 mol.) at ˜20-25° C. and then refluxed for 5 h. The reaction was cooled to ˜20-25° C. and (R)-Binol (55.2 g, 192 mmol) in CH₂Cl₂ (3.6 L) was added. The reaction mixture was stirred for 1 h, and then Ti(OⁱPr)₄ (158 g, 0.48 mol) was added. Then the mixture was stirred for another 1 h and a solution of 3,5-dimethylisoxazole-4-carbaldehyde (60 g, 0.48 mol, 1.0 eq) in CH₂Cl₂ (1.1 L) was added. The solution was stirred for 10 min, and then at rt an additional 16 h. Sat. NH4Cl solution (3 L) was added to the reaction and the mixture was filtered through a pad of Celite (500 g). The filter cake was washed with CH₂Cl₂ (2.1 L). The combined organic layers were washed with water (1 L), and brine (1 L), and concentrated under reduced pressure at ˜60-70° C. to give about 500 mL of a pale yellow liquid, which was treated with 1 L of n-heptane. A solid precipitated, which was filtered, and the filtrate was concentrated under reduced pressure at ˜60-70° C. to give about 300 mL of a pale yellow liquid (297 g, crude).

### b) (R)-1-(3,5-dimethylisoxazol-4-yl)prop-2-yn-1-ol

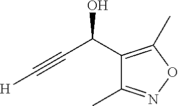

This intermediate was synthesized from (S)-1-(3,5-dimethylisoxazol-4-yl)-3-(triisopropylsilyl)prop-2-yn-1-ol essentially as described in preparation 22 (b) (45 g, yield: 62% for 2 steps).

### c) methyl 2-(4-hydroxy-3-iodophenyl)acetate

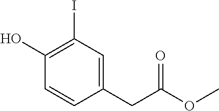

Methyl 2-(4-hydroxyphenyl)acetate (10.0 g, 0.060 mol) and NaI (10.0 g, 0.066 mol) were dissolved in DMF (50.0 mL), and the solution was cooled to −10° C. NaClO aqueous (60 mL) was added drop wise to the reaction mixture while keeping the temperature below 5° C. The mixture was stirred at −5˜5° C. for 10-25 min, and then quenched with a solution of 10% NaHSO3 aq. (100 mL, aq.) with stirring while keeping the temperature below 5° C., The yellow solution was stirred for 10 min at ˜10° C. and then the pH was adjusted to ˜2-3 with 2N HCl aqueous (40 mL). The mixture was extracted with EtOAc (100 mL, 50 mL, 50 mL), The combined extracts were washed with 10% NaHSO3 aq. (20 mL×2) and brine (20 mL*2), dried over Na2SO4 overnight, and concentrated under reduced pressure to give methyl 2-(4-hydroxy-3-iodophenyl)acetate (15.4 g, yield: 87.5%)

### d) (S)-methyl 2-(2-((3,5-dimethylisoxazol-4-yl)(hydroxy)methyl)benzofuran-5-yl)acetate

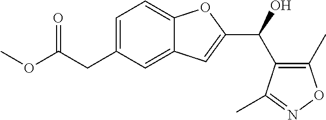

Methyl 2-(4-hydroxy-3-iodophenyl)acetate (78 g, 0.27 mol), Pd(PPh₃)₂Cl₂(1.85 g, 2.7 mmol), CuI (1 g, 5.3 mmol), and K₂CO₃(68.5 g, 0.49 mmol) were added to 800 mL of EtOAc and the reaction was degassed. The mixture was heated to 50-60° C. and (R)-1-(3,5-dimethylisoxazol-4-yl)prop-2-yn-1-ol (50 g, 0.33 mol) in EtOAc (200 mL) was dropped by syringe over 1 h. Then the mixture was stirring at 50-60° C. for 3 h and LCMS showed compound 7-I was 3%. Then the reaction was continued to stir at 50-60° C. for another 1 h. The reaction was then filtered through a pad of Celite (50 g) and the filter cake was washed with EtOAc (500 mL). The combined filtrates were washed with water (500 mL) and brine (500 mL), and concentrated under reduced pressure to give (S)-methyl 2-(2-((3,5-dimethylisoxazol-4-yl)(hydroxy)methyl)benzofuran-5-yl)acetate, which was carried through without further purification.

### e) (S)-2-(2-((3,5-dimethylisoxazol-4-yl)(hydroxy)methyl)benzofuran-5-yl)acetic acid

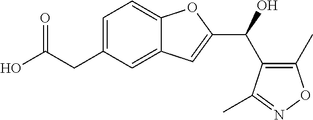 [SMILES: Cc1noc(C)c1[C@H](O)c1cc2cc(CC(=O)O)ccc2o1]

(S)-methyl 2-(2-((3,5-dimethylisoxazol-4-yl)(hydroxy)methyl)benzofuran-5-yl)acetate from step (d) was dissolved in 200 mL of THF at 15˜25° C. A solution of LiOH—H2O (17 g, 0.41 mol) in 50 mL of water was added and the mixture was stirred at 30±5° C. for 2 h. The mixture was then washed with EtOAc (30 mL*3) after 30.0 mL of water was added at rt. The organics were washed with water (30 mL*2). The combined aqueous layers were neutralized to pH ˜2-4 with 1N HCl aq. (˜30 mL) while a lot of pale yellow solid appeared. The aqueous was extracted with EtOAc (3*40 mL) and the organics were washed with water (20 mL) and brine (20 mL). The organics was concentrated under reduced pressure (<40° C.) to give yellow solid which was slurried in 25.0 mL of MTBE overnight at rt (10˜15° C.). and then filtered, washed with MTBE (10 mL×2) and dried to give (S)-2-(2-((3,5-dimethylisoxazol-4-yl)(hydroxy)methyl)benzofuran-5-yl)acetic acid as apale yellow solid (6.2 g, yield: 65%).

### Example 1

### N-((4-chlorophenyl)(phenyl)methyl)-2-(2-(3,5-dimethylisoxazol-4-yl)benzofuran-5-yl)acetamide

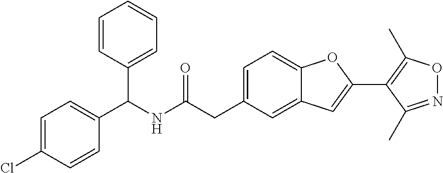 [SMILES: Cc1noc(C)c1-c1cc2cc(CC(=O)NC(c3ccccc3)c3ccc(Cl)cc3)ccc2o1]

### a) methyl 2-(4-hydroxyphenyl)acetate

To a solution of 2-(4-hydroxyphenyl)acetic acid (100 g, 658 mmol) in MeOH (1000 mL) was added dropwise conc. H₂SO₄ (40 mL) at 0° C. and the reaction was heated at 80° C. overnight. The reaction mixture was cooled to rt and MeOH was removed under reduced pressure. The resultant residue was dissolved in water (500 mL) and neutralized (pH=7) using saturated aq. NaHCO₃. The aqueous layer was extracted with EtOAc (3×500 mL). The combined organic layers were dried over Na₂SO₄ and concentrated under reduced pressure to afford the title compound (101 g, 92%) as a pale yellow solid. LCMS-P1: m/z=167.2 [M+H]⁺; Rₜ: 1.276 min.

### b) methyl 2-(3-formyl-4-hydroxyphenyl)acetate

To a solution of methyl 2-(4-hydroxyphenyl)acetate (30 g, 180 mmol) in CH₃CN (150 mL) was added MgCl₂ (33.8 g, 360 mmol) and Et₃N (72.6 g, 720 mmol) under N₂ and the reaction mixture was refluxed for 1 h. (CH₂O)ₙ was added and the reaction was refluxed overnight. After cooling to rt, Et₂O (200 mL) was added, and 1 M aq. HCl (300 mL) was added. The organic layer was separated and washed with 1 M aq. HCl (3×300 mL), dried over Na₂SO₄, and concentrated. The resultant residue was purified by column chromatography (5:1 petroleum ether/EtOAC) to obtain the title compound (21.6 g, 62%) as a yellow oil. LCMS-P1: m/z=195 [M+H]⁺; Rₜ: 1.352 min.

### c) methyl 2-(3-(2,2-dibromovinyl)-4-hydroxyphenyl)acetate

To a solution of CBr₄ (23 g, 70 mmol) in CH₂Cl₂ (100 mL) was added a solution of PPh₃ (27.5 g, 105 mmol) in CH₂Cl₂ (50 mL) at 0° C. and the reaction mixture was stirred for 15 min at the same temperature. A solution of methyl 2-(3-formyl-4-hydroxyphenyl)acetate (7 g, 35 mmol) in CH₂Cl₂ (25 mL) and Et₃N (10.6 g, 105 mmol) was added at 0° C. over 1 h. Following the addition, the reaction mixture was stirred for another 2 h. Water (150 mL) was added to the reaction mixture slowly, followed by extraction with CH₂Cl₂ (3×200 mL). The organic layer was dried over Na₂SO₄ and concentrated. The resultant residue was purified by column chromatography (10:1 petroleum ether/EtOAc) to obtain the title compound (4.6 g, 38%) as a yellow oil. LCMS-P1: m/z=349 [M+H]⁺; Rₜ: 1.625 min.

### d) methyl 2-(2-bromobenzofuran-5-yl)acetate

To a solution of methyl 2-(3-(2,2-dibromovinyl)-4-hydroxyphenyl)acetate (2.7 g, 7.75 mmol) in THF (200 mL) were added K₃PO₄ (3.28 g, 15.5 mmol) and CuI (59 mg, 0.31 mmol) under N₂. The reaction mixture was stirred at 80° C. for 12 h. Water (100 mL) was added to the reaction mixture, followed by extraction with CH₂Cl₂ (3×100 mL). The combined organic layers were dried over Na₂SO₄ and concentrated. The resultant residue was purified by column chromatography (50:1 petroleum ether/EtOAc) to obtain the title compound (1.525 g, 73%) as a yellow solid. LCMS-P1: m/z=269/271 [M+H]⁺; Rₜ: 1.670 min. ¹H NMR (400 MHz, DMSO-d₆) δ ppm 7.54 (d, 1H), 7.50 (d, 1H), 7.21 (d, 1H), 7.11 (s, 1H), 3.77 (s, 2H), 3.61 (s, 3H).

### e) methyl 2-(2-(3,5-dimethylisoxazol-4-yl)benzofuran-5-yl)acetate

To a solution of methyl 2-(2-bromobenzofuran-5-yl)acetate (1.00 g, 3.71 mmol) in degassed toluene (10 mL) at 25° C. were added K₂CO₃ (1.5 g, 11.3 mmol) as a solution in water (10 mL), 3,5-dimethylisoxazol-4-ylboronic acid (0.624 g, 4.45 mmol) as a solution in EtOH (10 mL), and Pd(PPh₃)₄ (0.43 g, 0.371 mmol). The reaction mixture was stirred for 10 min under Ar atmosphere. The reaction mixture was then heated to 80° C. and stirred for 4 h under Ar atmosphere at the same temperature. The reaction mixture was concentrated, diluted with EtOAc (25 mL), and washed with water (25 mL). The reaction mixture was dried over Na₂SO₄, concentrated, and purified by column chromatography (1% EtOAc/hexanes) to provide the title compound (0.650 g, 61%).

### f) 2-(2-(3,5-dimethylisoxazol-4-yl)benzofuran-5-yl)acetic acid

To a solution of methyl 2-(2-(3,5-dimethylisoxazol-4-yl)benzofuran-5-yl)acetate (0.65 g, 2.280 mmol) in MeOH (35 mL) was added dropwise NaOH (0.273 g, 6.82 mmol) in water (15 mL) and the reaction mixture was stirred at rt for 2 h. MeOH was distilled off under reduced pressure and water (25 mL) was added to the reaction mixture. The aqueous layer was washed with Et₂O and then acidified to pH=4 using 6.0 M HCl. The resultant precipitate was filtered and dried under reduced pressure to provide the title compound (0.40 g, 64%). ¹H NMR (400 MHz, DMSO-d₆) δ ppm 12.32 (s, 1H), 7.55 (d, 2H), 7.21 (d, 1H), 7.08 (s, 1H), 3.67 (s, 2H), 2.68 (s, 3H), 2.45 (s, 3H).

### g) N-((4-chlorophenyl)(phenyl)methyl)-2-(2-(3,5-dimethylisoxazol-4-yl)benzofuran-5-yl)acetamide

To a solution of 2-(2-(3,5-dimethylisoxazol-4-yl)benzofuran-5-yl)acetic acid (0.10 g, 0.369 mmol) in THF (5 mL) was added portionwise EDC (0.106 g, 0.553 mmol) and the reaction mixture was stirred at rt for 2 h. (4-chlorophenyl)(phenyl)methanamine (0.103 g, 0.405 mmol), HOBt (0.028 g, 0.184 mmol), and Et₃N (0.153 mL, 1.107 mmol) were added and the reaction mixture was stirred for 24 h at rt. Water (10 mL) was added to the reaction mixture, followed by extraction with EtOAc (2×25 mL). The organic layer was dried over Na₂SO₄ and concentrated. The resultant residue was purified by column chromatography (30% EtoAc/hexane) to provide the title compound (0.070 g, 39%). LCMS-X1: m/z=471.4 [M+H]⁺; Rₜ: 7.20 min. ¹H NMR (400 MHz, DMSO-d₆) δ ppm 9.07 (d, 1H), 7.54 (d, 2H), 7.31-7.40 (m, 2H), 7.22-7.28 (m, 8H), 7.01 (s, 1H), 6.11 (d, 1H), 3.64 (s, 2H), 2.67 (s, 3H), 2.45 (s, 3H).

### Example 2

### 2-(2-(3,5-dimethylisoxazol-4-yl)benzofuran-5-yl)-N-(1-(2,4-dimethylphenyl)-4-methylpentyl)acetamide

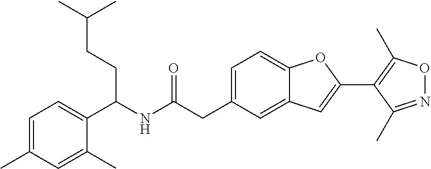 [SMILES: Cc1ccc(C(CCC(C)C)NC(=O)Cc2ccc3oc(-c4c(C)noc4C)cc3c2)c(C)c1]

### a) 1-(2,4-dimethylphenyl)-4-methylpentan-1-amine

To a solution of 4-methylpentanenitrile (1.0 g, 10.3 mmol) in THF (40 mL) was added (2,4-dimethylphenyl)magnesium bromide (16 mL, 12.4 mmol, 0.78 N in THF) at 0° C. under N₂. The reaction mixture was allowed to warm up to rt, and stirred for 2 h. MeOH (10 mL) was added to the reaction mixture followed by NaBH₄ (391 mg, 10.3 mmol). After stirring at rt for an additional 2 h, most of the organic solvent was removed under reduced pressure, and water (30 mL) was added to the reaction mixture. The reaction mixture was extracted with EtOAc (3×30 mL). The combined organic layers were washed with brine, dried over Na₂SO₄, and concentrated. The resultant residue was purified by flash chromatography (3:1 to 1:1 petroleum ether/EtOAc) to afford the title compound (202 mg, 10%). LC-MS: 189 [M−NH₂]⁺; Rₜ: 1.32 min.

### b) 2-(2-(3,5-dimethylisoxazol-4-yl)benzofuran-5-yl)-N-(1-(2,4-dimethylphenyl)-4-methylpentyl)acetamide

To a solution of 2-(2-(3,5-dimethylisoxazol-4-yl)benzofuran-5-yl)acetic acid (45 mg, 0.166 mmol) in CH₂Cl₂ (10 mL) were added 1-(2,4-dimethylphenyl)-4-methylpentan-1-amine (37 mg, 0.18 mmol), EDC (64 mg, 0.332 mmol), HOBt (45 mg, 0.332 mmol), and DIPEA (86 mg, 0.664 mmol). The reaction mixture was stirred at rt overnight. The reaction mixture was diluted with water (30 mL) and extracted with CH₂Cl₂ (3×30 mL). The combined organic layers were washed with 1% aq. HCl dried over Na₂SO₄, and concentrated. The resultant residue was purified by Preparatory TLC (1:3 EtOAc/petroleum ether) to provide the title compound (25 mg, 33%) as a white solid. LC-MS-P1: m/z=459.3 [M+H]⁺; Rₜ: 1.92 min. ¹H NMR (400 MHz, MeOH-d₄) δ ppm 7.52 (s, 1H), 7.44 (d, J=8.4 Hz, 1H), 7.23 (m, 1H), 7.16 (d, J=8.0 Hz, 1H), 6.97 (m, 1H), 6.88 (s, 1H), 5.03 (t, 1H), 3.60 (q, J=6.0 Hz, 2H), 2.68 (s, 3H), 2.48 (s, 3H), 2.30 (s, 3H), 2.26 (s, 3H), 1.75-1.67 (m, 2H), 1.56-1.48 (m, 1H), 1.31-1.48 (m, 2H), 0.85 (t, 6H).

### Example 3

### N-((2,4-dimethylphenyl)(phenyl)methyl)-2-(2-(pyridin-3-yl)benzofuran-5-yl)acetamide

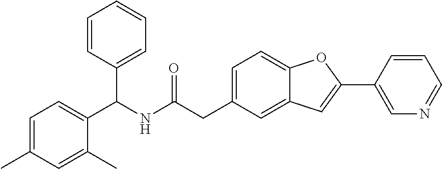 [SMILES: Cc1ccc(C(NC(=O)Cc2ccc3oc(-c4cccnc4)cc3c2)c2ccccc2)c(C)c1]

### a) methyl 2-(2-(pyridin-3-yl)benzofuran-5-yl)acetate

To a solution of methyl 2-(2-bromobenzofuran-5-yl)acetate (0.10 g, 0.37 mmol) in degassed toluene (3 mL) and EtOH (3 mL) at 25° C. were added a solution of Na₂CO₃ (0.118 g, 1.114 mmol) in water (10 mL), pyridin-3-ylboronic acid (0.06 g, 0.48 mmol), and Pd(dppf)Cl₂.CH₂Cl₂ complex (0.031 g, 0.037 mmol). The reaction was stirred for 10 min under Ar atmosphere, followed by heating to 80° C. for 6 h. The reaction mixture was concentrated, diluted with EtOAc (25 mL), washed with water (25 mL), dried over Na₂SO₄, and concentration. The resultant residue was purified by column chromatography (20% EtOAc/hexanes) to provide the title compound (0.060 g, 57%).

### b) 2-(2-(pyridin-3-yl)benzofuran-5-yl)acetic acid

To a solution of methyl 2-(2-(pyridin-3-yl)benzofuran-5-yl)acetate (0.65 g, 2.313 mmol) was added dropwise a solution of NaOH (0.185 g, 4.62 mmol) in water (12 mL) and the reaction mixture was stirred at 80° C. for 30 min. The reaction mixture was then cooled to 0° C. and acidified with 6 N HCl. The resultant precipitate was filtered, washed with hexanes, and dried under vacuum to provide the title compound (0.20 g, 34%). ¹H NMR (400 MHz, DMSO-d₆) δ ppm 9.14 (s, 1H), 8.59 (d, 1H), 8.27 (d, 1H), 7.52-7.59 (m, 4H), 7.24 (d, 1H), 3.69 (s, 2H).

### c) (2,4-dimethylphenyl)(phenyl)methanamine hydrochloride

To a suspension of Mg (1.32 g, 55 mmol) in THF (60 mL) was added a catalytic amount of I₂ and 1-bromo-2,4-dimethylbenzene (0.50 g, 2.7 mmol). The reaction was initiated by heating and additional 1-bromo-2,4-dimethylbenzene (8.75 g, 47.3 mmol) was added dropwise. The reaction mixture was stirred at rt for 4 h under N₂. Benzonitrile (5.15 g, 50 mmol) was added dropwise to the Grignard system and the reaction mixture was stirred at rt for 16 h. The reaction was quenched by the addition of MeOH (20 mL), followed by portionwise addition of NaBH₄ (1.9 g, 50 mmol). After stirring at rt for 5 h, the reaction was quenched by the addition of water (20 mL). Solvent was removed under reduced pressure. The residue was extracted with EtOAc (3×100 mL). The organic layer was washed with 1 N HCl and the resultant precipitate was collected by filtration to afford the title compound (8.8 g, 84%) as a white solid. LC-MS-P1: 195 [M-NH₂]⁺; Rₜ: 1.232 min. ¹H NMR (500 MHz, DMSO-d₆) δ ppm 9.15 (s, 2H), 7.55 (d, J=8.0 Hz, 1H), 7.45-7.32 (m, 5H), 7.12 (d, J=8.0 Hz, 1H), 7.04 (s, 1H), 5.64-5.62 (m, 1H), 2.27 (s, 3H), 2.22 (s, 3H).

### d) N-((2,4-dimethylphenyl)(phenyl)methyl)-2-(2-(pyridin-3-yl)benzofuran-5-yl)acetamide

To a solution of 2-(2-(pyridin-3-yl)benzofuran-5-yl)acetic acid (0.10 g, 0.395 mmol) in THF (5 mL), was added EDC (0.113 g, 0.592 mmol) and the reaction mixture was stirred at rt for 30 min. (2,4-dimethylphenyl)(phenyl)methanamine hydrochloride (0.10 g, 0.474 mmol), HOBt (0.060 g, 0.395 mmol), and Et₃N (0.153 mL, 1.107 mmol) were added and the reaction mixture was stirred for 24 h at rt. Ice cold water (10 mL) was added and the resultant precipitate was filtered and dried under vacuum to provide the title compound (0.012 g, 6.8%). LCMS-X1: m/z=447.3 [M+H]⁺; Rₜ: 6.69 min. ¹H NMR (400 MHz, DMSO-d₆) δ ppm 9.14 (s, 1H), 8.92 (d, 1H), 8.60 (d, 1H), 8.27 (d, 1H), 7.52-7.59 (m, 4H), 7.22-7.33 (m, 4H), 7.17-7.18 (m, 2H), 6.95-7.00 (m, 3H), 6.19 (d, 1H), 3.62 (s, 2H), 2.18 (s, 3H), 2.15 (s, 3H).

### Example 4

### 2-(2-(3,5-dimethylisoxazol-4-yl)benzofuran-5-yl)-N-((2,4-dimethylphenyl)(phenyl)methyl)-2-methylpropanamide

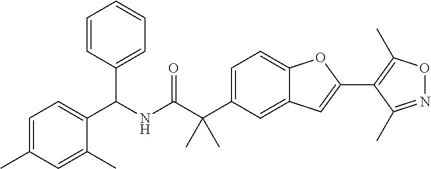 [SMILES: Cc1ccc(C(NC(=O)C(C)(C)c2ccc3oc(-c4c(C)noc4C)cc3c2)c2ccccc2)c(C)c1]

### a) methyl 2-(4-methoxyphenyl)acetate

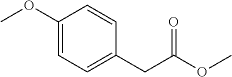

To a solution of 2-(4-methoxyphenyl)acetic acid (10.0 g, 60.24 mmol) in MeOH (80 mL) at 0° C. was added dropwise conc. H₂SO₄ (5 mL). The reaction was then refluxed at 80° C. overnight. The reaction mixture was cooled to rt and MeOH was distilled out under reduced pressure. The resultant residue was dissolved in water and neutralized (pH=7) using saturated aq. NaHCO₃. The aqueous layer was extracted with EtOAc (3×100 mL), dried over Na₂SO₄, and distilled under reduced pressure to provide the title compound (11.0 g, 93%). ¹H NMR (400 MHz, CDCl₃) δ ppm 7.22 (d, 2H), 6.87-6.90 (m, 2H), 3.81 (s, 3H), 3.71 (s, 2H), 3.59 (s, 3H).

### b) methyl 2-(4-methoxyphenyl)-2-methylpropanoate

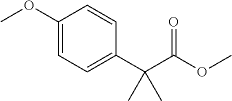

To a solution of methyl 2-(4-methoxyphenyl)acetate (10.0 g, 55.49 mmol) in THF (100 mL) at −78° C. was added methyl iodide (23.64 g, 166.5 mmol) very slowly. KOt-Bu (18.68 g, 166.5 mmol) was then added portionwise over 30 min and the reaction mixture was stirred at −78° C. for 1 h followed by rt for another 1 h. The reaction was quenched by the addition of water (25 mL) and extracted with EtOAc (2×250 mL). The organic layer was dried over Na₂SO₄ and concentrated to obtain the title compound (10.46 g, 91%).

### c) 2-(4-hydroxyphenyl)-2-methylpropanoic acid

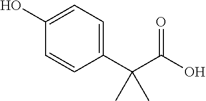

To a solution of methyl 2-(4-methoxyphenyl)acetate (10.0 g, 48.00 mmol) in MeOH (20 mL) was added dropwise a solution of NaOH (7.69 g, 192.3 mmol) in water (50 mL). The reaction mixture was stirred at rt for 6 h. The MeOH was distilled off at reduced pressure and water (25 mL) was added. The aqueous layer was washed with Et₂O and then acidified to pH=4 using 6.0 M HCl. The resultant precipitate was filtered, washed with hexanes, and dried. The resultant solid was dissolved in CH₂Cl₂ (150 mL) and cooled to −78° C. followed by the addition of BBr₃ (1.0 M solution in CH₂Cl₂) (72.0 mL, 72.00 mmol) over 30 min. After the completion of the addition, the temperature was raised to 0° C., and the reaction mixture was stirred for an additional 30 min Water was slowly added and the organic layer was separated, dried over Na₂SO₄, and concentrated to provide the title compound (4.30 g, 50%). ¹H NMR (400 MHz, DMSO-d₆) δ ppm 12.17 (br s, 1H), 9.31 (s, 1H), 7.14 (d, 2H), 6.71 (d, 2H), 1.42 (s, 6H).

### d) methyl 2-(4-hydroxyphenyl)-2-methylpropanoate

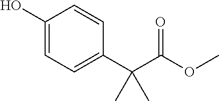

To a solution of 2-(4-hydroxyphenyl)-2-methylpropanoic acid (5.0 g, 27.70 mmol) in MeOH (50 mL) at 0° C. was added dropwise conc. H₂SO₄ (3 mL). The reaction mixture was then refluxed at 80° C. for 3 h. The reaction mixture was then cooled to rt and MeOH was distilled out under reduced pressure. The resultant residue was dissolved in water and neutralized (pH=7) using saturated aq. NaHCO₃. The aqueous layer was extracted with EtOAc (3×50 mL), dried over Na₂SO₄, and distilled under reduced pressure to provide the title compound (4.80 g, 89%). ¹H NMR (400 MHz, CDCl₃) δ ppm 7.10 (d, 2H), 6.71 (dd, 2H), 3.56 (s, 3H), 1.44 (s, 6H).

### e) methyl 2-(3-formyl-4-hydroxyphenyl)-2-methylpropanoate

To a solution of methyl 2-(4-hydroxyphenyl)-2-methylpropanoate (4.8 g, 24.7 mmol) in TFA (96 mL) at 0° C. was added hexamine (1.38 g, 9.88 mmol) over 10 min and the reaction mixture was warmed to rt followed by heating to 80° C. for 4-6 h. TFA was removed at reduced pressure. The reaction mixture was then diluted with water (50 mL) and neutralized with aq. NaHCO₃ (pH=8). The reaction mixture was extracted with CH₂Cl₂ (3×100 mL). The combined organic layers were dried over Na₂SO₄ and concentration. The resultant residue was purified by column chromatography (8% EtOAc/hexanes) to provide the title compound (1.48 g, 27%). ¹H NMR (400 MHz, DMSO-d₆) δ ppm 10.71 (s, 1H), 10.25 (s, 1H), 7.59 (d, 1H), 7.48, (dd, 1H), 6.98 (d, 1H), 3.58 (s, 3H), 1.49 (s, 6H).

### f) methyl 2-(3-(2,2-dibromovinyl)-4-hydroxyphenyl)-2-methylpropanoate

The title compound was synthesized from methyl 2-(3-formyl-4-hydroxyphenyl)-2-methylpropanoate following essentially the procedure of Example 1(c) (2.0 g, 80%). ¹H NMR (400 MHz, DMSO-d₆) δ ppm 9.97 (s, 1H), 7.60 (s, 1H), 7.53-7.54, (d, 1H), 7.16 (dd, 1H), 6.83 (d, 1H), 3.57 (s, 3H), 1.46 (s, 6H).

### g) methyl 2-(2-bromobenzofuran-5-yl)-2-methylpropanoate

The title compound was synthesized from methyl 2-(3-(2,2-dibromovinyl)-4-hydroxyphenyl)-2-methylpropanoate following essentially the procedure of Example 1(d) (0.96 g, 61%). ¹H NMR (400 MHz, DMSO-d₆) δ ppm 7.53-7.55 (m, 2H), 7.25 (dd, 1H), 7.10 (s, 1H), 3.58 (s, 3H), 1.54 (s, 6H).

### h) methyl 2-(2-(3,5-dimethylisoxazol-4-yl)benzofuran-5-yl)-2-methylpropanoate

The title compound was synthesized from methyl 2-(2-bromobenzofuran-5-yl)-2-methylpropanoate following essentially the procedure of Example 1(e), except Na₂CO₃ was used (0.600 g, 63%). ¹H NMR (400 MHz, DMSO-d₆) δ ppm 7.56-7.60 (m, 2H), 7.26 (dd, 1H), 7.08 (s, 1H), 3.59 (s, 3H), 2.67 (s, 3H), 1.57 (s, 6H).

### i) 2-(2-(3,5-dimethylisoxazol-4-yl)benzofuran-5-yl)-2-methylpropanoic acid

The title compound was synthesized from methyl 2-(2-(3,5-dimethylisoxazol-4-yl)benzofuran-5-yl)-2-methylpropanoate following essentially the procedure of Example 1(f) (0.35 g, 64%). ¹H NMR (400 MHz, DMSO-d₆) δ ppm 12.35 (br s, 1H), 7.63 (d, 1H), 7.57 (d, 1H), 7.31 (dd, 1H), 7.09 (s, 1H), 2.67 (s, 3H), 2.44 (s, 3H), 1.54 (s, 6H).

### j) 2-(2-(3,5-dimethylisoxazol-4-yl)benzofuran-5-yl)-N-((2,4-dimethylphenyl)(phenyl)methyl)-2-methylpropanamide

To a solution of 2-(2-(3,5-dimethylisoxazol-4-yl)benzofuran-5-yl)-2-methylpropanoic acid (0.05 g, 0.16 mmol) in DMF (2 mL), EDC (0.04 g, 0.20 mmol), HOBt (0.03 g, 0.200 mmol), DMAP (0.03 g, 0.033 mmol), and (2,4-dimethylphenyl)(phenyl)methanamine (0.04 g, 0.18 mmol) were added and the reaction mixture was stirred overnight at rt. Water (50 mL) was added and the reaction mixture was extracted with EtOAc (100 mL). The organic layer was dried over Na₂SO₄, concentrated, and purified by column chromatography (0.8% MeOH/CH₂Cl₂) to provide the title compound (33 mg, 40%). LCMS-X1: m/z=469.3 [M+H]⁺; Rₜ: 5.62 min. ¹H NMR (400 MHz, DMSO-d₆) δ ppm 7.96 (d, 1H), 7.53-7.57 (m, 2H), 7.18-7.27 (m, 4H), 7.05 (d, 3H), 6.95 (s, 1H), 6.80-6.87 (m, 2H), 6.28 (d, 1H), 2.67 (s, 3H), 2.33 (s, 3H), 2.46 (s, 3H), 1.46 (s, 3H), 1.09 (s, 6H).

### Example 5

### 2-(2-(3,5-dimethylisoxazol-4-yl)benzofuran-5-yl)-N-(phenyl(p-tolyl)methyl)acetamide

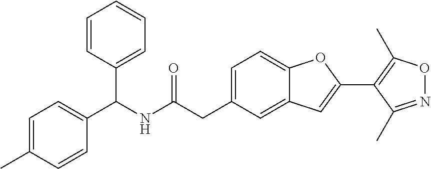 [SMILES: Cc1ccc(C(NC(=O)Cc2ccc3oc(-c4c(C)noc4C)cc3c2)c2ccccc2)cc1]

To a stirred solution of 2-(2-(3,5-dimethylisoxazol-4-yl)benzofuran-5-yl)acetic acid (60 mg, 0.22 mmol) in dry DMF (3 mL) were added HATU (100 mg, 0.26 mmol) followed by (4-methylphenyl)(phenyl)methanamine (43 mg, 0.22 mmol) in dry DMF (2 mL) and NMM (0.1 mL, 0.66 mmol) at 0° C. The reaction mixture was warmed slowly to rt and stirred for an additional 3 h. The reaction mixture was diluted with EtOAc. The organic layer was washed with 10% aq. NaHCO₃ solution, water, and brine. The organic layer was dried over anhydrous Na₂SO₄, filtered, and concentrated under reduced pressure. The resultant residue was purified by column chromatography (30% EtOAc/petroleum ether) followed by preparative TLC (30% EtOAc/petroleum ether) to afford the title compound (10 mg, 10%). TLC: 40% EtOAc/petroleum ether, R_{f}=0.3. LCMS-G12: m/z=452.0 [M+H]⁺; Rₜ: 5.39 min. ¹H NMR (400 MHz, DMSO-d₆) δ ppm 8.99 (d, J=8.5 Hz, 1H), 7.54-7.52 (m, 2H), 7.32-7.29 (m, 2H), 7.25-7.22 (m, 4H), 7.16-7.10 (m, 4H), 7.06 (s, 1H), 6.05 (d, J=8.5 Hz, 1H), 3.63 (s, 2H), 2.67 (s, 3H), 2.44 (s, 3H), 2.25 (s, 3H).

### Example 6

### N-((2,4-dichlorophenyl)(phenyl)methyl)-2-(2-(3,5-dimethylisoxazol-4-yl)benzofuran-5-yl)acetamide

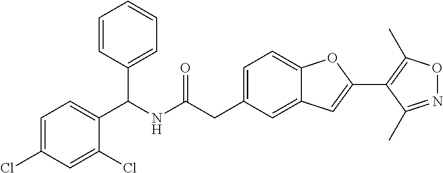 [SMILES: Cc1noc(C)c1-c1cc2cc(CC(=O)NC(c3ccccc3)c3ccc(Cl)cc3Cl)ccc2o1]

The title compound was synthesized from 2-(2-(3,5-dimethylisoxazol-4-yl)benzofuran-5-yl)acetic acid and (2,4-dichlorophenyl)(phenyl)methanamine following essentially the procedure of Example 5 (20 mg, 18%). TLC: 40% EtOAc/petroleum ether, R_{f}=0.4. LCMS-G12: m/z=505.4 [M+H]⁺; Rₜ: 6.33 min. ¹H NMR (400 MHz, DMSO-d₆) δ ppm 9.10 (d, J=8.2 Hz, 1H), 7.61 (d, J=1.8 Hz, 1H), 7.55-7.52 (m, 2H), 7.49-7.41 (m, 2H), 7.36-7.32 (m, 2H), 7.30-7.26 (m, 1H), 7.22 (dd, J=8.4, 1.7 Hz, 1H), 7.18-7.16 (m, 2H), 7.07 (s, 1H), 6.34 (d, J=7.9 Hz, 1H), 3.63 (s, 2H), 2.67 (s, 3H), 2.44 (s, 3H).

### Example 7

### N-((4-chlorophenyl)(phenyl)methyl)-2-(2-(3,5-dimethylisoxazol-4-yl)benzofuran-5-yl)-2-methylpropanamide

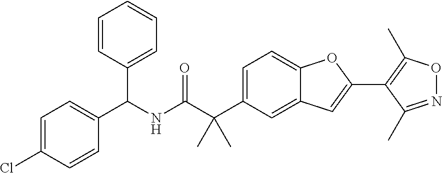 [SMILES: Cc1noc(C)c1-c1cc2cc(C(C)(C)C(=O)NC(c3ccccc3)c3ccc(Cl)cc3)ccc2o1]

The title compound was synthesized from 2-(2-(3,5-dimethylisoxazol-4-yl)benzofuran-5-yl)-2-methylpropanoic acid and (4-chlorophenyl)(phenyl)methanamine hydrochloride following essentially the procedure of Example 2(b) (10 mg, 11.8%). LCMS-P1: m/z 499 [M+H]⁺; Rₜ: 2.20 min. ¹H NMR (400 MHz, MeOD-d₄) δ ppm 7.56 (d, J=1.6 Hz, 1H), 7.47 (d, J=8.8 Hz, 1H), 7.24-7.29 (m, 7H), 7.09-7.10 (m, 4H), 6.88 (s, 1H), 6.34 (s, 1H), 2.70 (s, 3H), 2.50 (s, 3H), 1.64 (m, 6H).

### Example 8

### N-((4-chloro-2-methylphenyl)(phenyl)methyl)-2-(2-(3,5-dimethylisoxazol-4-yl)benzofuran-5-yl)-2-methylpropanamide

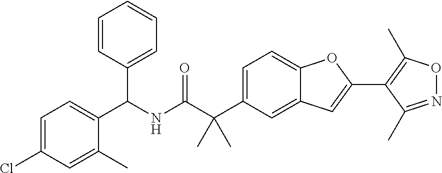 [SMILES: Cc1cc(Cl)ccc1C(NC(=O)C(C)(C)c1ccc2oc(-c3c(C)noc3C)cc2c1)c1ccccc1]

### (a) (4-chloro-2-methylphenyl)(phenyl)methanamine

This compound was synthesized from 4-chloro-2-methylbenzonitrile and phenylmagnesium bromide essentially as described in example 2 (a) (0.16 g, 20.83%). LC-MS: 215.0 [M−NH2]⁺; Rt=1.261 min. ¹H NMR (400 MHz, DMSO-d₆) δ ppm 7.59-7.61 (d, 1H), 7.24-7.32 (m, 4H), 7.15-7.19 (q, 3H), 5.37 (s, 1H), 2.31-2.34 (t, 1H), 2.26 (s, 3H).

### b) N-((4-chloro-2-methylphenyl)(phenyl)methyl)-2-(2-(3,5-dimethylisoxazol-4-yl)benzofuran-5-yl)-2-methylpropanamide

The title compound was synthesized from 2-(2-(3,5-dimethylisoxazol-4-yl)benzofuran-5-yl)-2-methylpropanoic acid and (4-chloro-2-methylphenyl)(phenyl)methanamine hydrochloride following essentially the procedure of Example 2(b) (18 mg, 21%). LCMS-P1: m/z 513 [M+H]⁺; Rₜ: 2.24 min. ¹H NMR (400 MHz, MeOD-d₄) δ ppm 7.57 (d, J=1.6 Hz, 1H), 7.48 (d, J=8.8 Hz 1H), 7.03-7.29 (m, 5H), 7.03 (m, 3H), 6.83-6.88 (m, 2H), 6.34 (s, 1H), 2.69 (s, 3H), 2.48 (s, 3H), 2.20 (s, 3H), 1.62 (m, 6H).

### Example 9

### 2-(2-(3,5-dimethylisoxazole-4-carbonyl)benzofuran-5-yl)-N-((2,4-dimethylphenyl)(phenyl)methyl)acetamide

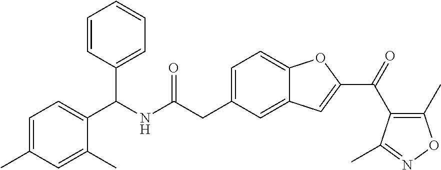

### a) methyl 3,5-dimethylisoxazole-4-carboxylate

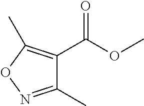

To a solution of 3,5-dimethylisoxazole-4-carboxylic acid (9.2 g, 65.2 mmol) in MeOH (50 mL) was added SOCl₂ (15.3 g, 130.4 mmol) very slowly. The reaction mixture was heated to 70° C. overnight. The reaction mixture was then cooled to rt, concentrated, and purified by column chromatography (10% EtOAc/petroleum ether) to afford the title compound (9.0 g, 89%). LCMS-P1: 156 [M+H]⁺; Rₜ: 1.404 min.

### b) (3,5-dimethylisoxazol-4-yl)methanol

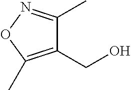

To a stirred solution of methyl 3,5-dimethylisoxazole-4-carboxylate (9.0 g, 58 mmol) in THF (200 mL) at 0° C. was added LiAlH₄ (2.42 g, 63.8 mmol) portionwise. After addition, the reaction mixture was allowed to warm up to rt and stirred overnight. The reaction mixture was quenched by the successive addition of water (2.5 mL), 10% aq. NaOH (5 mL), and water (7.5 mL). The organic layer was separated, dried over Na₂SO₄, filtered, and concentrated to afford the title compound (5.0 g, 68%). LCMS-P1: 128 [M+H]⁺; Rₜ: 0.963 min. ¹H NMR (400 MHz, CDCl₃) δ ppm 4.37 (s, 2H), 2.30 (s, 3H), 2.20 (s, 3H).

### c) 3,5-dimethylisoxazole-4-carbaldehyde

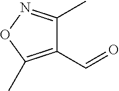

To a solution of (3,5-dimethylisoxazol-4-yl)methanol (1.00 g, 7.86 mmol) in CH₂Cl₂ (20 mL) at 0° C. was added Dess-Martin periodinane (4.17 g, 9.83 mmol) very slowly over 10 min and the reaction mixture was warmed to rt. The reaction mixture was stirred at rt for 60 min and then filtered through Celite and washed through with CH₂Cl₂. The organic layer was dried over Na₂SO₄, concentrated, and purified by column chromatography (15% EtOAc/hexanes) to provide the title compound (0.450 g, 46%). ¹H NMR (400 MHz, DMSO-d₆) δ ppm 9.92 (s, 1H), 2.68 (s, 3H), 2.37 (s, 3H).

### d) 1-(3,5-dimethylisoxazol-4-yl)ethanol

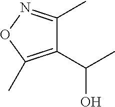

To a solution of 3,5-dimethylisoxazole-4-carbaldehyde (0.450 g, 0.36 mmol) in dry THF (3 mL) at 0° C. was added MeMgBr (3.0 M solution in Et₂O) (1.2 mL, 3.60 mmol) dropwise over 10 min and the reaction mixture was warmed to rt. The reaction mixture was stirred at rt for 90 min. Water (10 mL) was added very slowly and the reaction mixture was extracted with EtOAc (100 mL). The organic layer was dried over Na₂SO₄, concentrated, and purified by column chromatography (27% EtOAc/hexanes) to provide the title compound (0.480 g, 95%). ¹H NMR (400 MHz, DMSO-d₆) δ ppm 5.07 (d, 1H), 4.65-4.70 (m, 1H), 2.33 (s, 3H), 2.20 (s, 3H), 1.32 (d, 3H).

### e) 1-(3,5-dimethylisoxazol-4-yl)ethanone

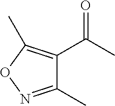

To a solution of 1-(3,5-dimethylisoxazol-4-yl)ethanol (0.30 g 2.12 mmol) in CH₂Cl₂ (8 mL) at 0° C. was added PCC (0.068 g, 3.19 mmol) and reaction was stirred for 1 h at rt. Water (25 mL) was added slowly and the reaction mixture was extracted with CH₂Cl₂ (3×10 mL). The organic layer was dried over Na₂SO₄, filtered, concentrated, and purified by column chromatography (10% EtOAc/hexane) to provide the title compound (0.10 g, 34%). ¹H NMR (400 MHz, CDCl₃) δ ppm 2.68 (s, 3H), 2.44 (s, 5H), 2.24 (s, 1H).

### f) 2-bromo-1-(3,5-dimethylisoxazol-4-yl)ethanone

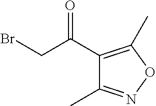

To a solution of 1-(3,5-dimethylisoxazol-4-yl)ethanone (1.0 g, 7.13 mmol) in CCl₄ (40 mL) was added AcOH (1.0 g, 1.142 mmol). The reaction mixture was stirred at 48° C., followed by dropwise addition of Br₂ (0.37 mL, 7.19 mmol) in CCl₄ (30 mL). The reaction mixture was stirred at 48° C. for an additional 20 min. Ice cold water (50 mL) was added and the reaction mixture was extracted with CH₂Cl₂ (3×25 mL). The organic layer was dried over Na₂SO₄, filtered, concentrated, and purified by column chromatography (5% EtOAc/hexane) to provide the title compound (0.80 g, 51%). ¹H NMR (400 MHz, CDCl₃) δ ppm 4.18 (s, 2H), 2.74 (s, 3H), 2.52 (s, 3H).

### g) methyl 2-(2-(3,5-dimethylisoxazole-4-carbonyl)benzofuran-5-yl)acetate

To a solution of methyl 2-(3-formyl-4-hydroxyphenyl)acetate (0.622 g, 3.21 mmol) in DMF (5 mL) at 10° C. was added K₂CO₃ (0.886 g, 6.42 mmol) and the reaction mixture was stirred for 30 min. A solution of 2-bromo-1-(3,5-dimethylisoxazol-4-yl)ethanone (0.70 g, 3.21 mmol) in DMF (2 mL) was added and the reaction mixture was warmed to rt. The reaction mixture was stirred at rt for 15 h. Water (10 mL) was added and the reaction mixture was extracted with EtOAc (3×25 mL), washed with brine (25 mL), dried over Na₂SO₄, filtered, concentrated, and purified using column chromatography (5% EtOAc/hexanes) to obtain the title compound (0.80 g, 71%) as a white solid. ¹H NMR (400 MHz, CDCl₃) δ ppm 7.68 (s, 1H), 7.57 (d, 2H), 7.45-7.47 (m, 2H), 3.76 (d, 5H), 2.56 (s, 3H), 2.41 (s, 3H).

### h) 2-(2-(3,5-dimethylisoxazole-4-carbonyl)benzofuran-5-yl)acetic acid

The title compound was synthesized from methyl 2-(2-(3,5-dimethylisoxazole-4-carbonyl)benzofuran-5-yl)acetate following essentially the procedure of Example 1(f) (0.080 g, 47%). ¹H NMR (400 MHz, DMSO-d₆) δ ppm 12.41 (s, 1H), 7.72 (d, 2H), 7.48 (d, 2H), 4.04 (s, 2H), 2.52 (s, 3H), 2.22 (s, 3H).

### i) 2-(2-(3,5-dimethylisoxazole-4-carbonyl)benzofuran-5-yl)-N-((2,4-dimethylphenyl)(phenyl)methyl)acetamide

The title compound was synthesized from 2-(2-(3,5-dimethylisoxazole-4-carbonyl)benzofuran-5-yl)acetic acid and (2,4-dimethylphenyl)(phenyl)methanamine following essentially the procedure of Example 1(g) (0.080 g, 47%). LCMS-X1: m/z=492.9 [M+H]⁺; Rₜ: 4.16 min. ¹H NMR (400 MHz, DMSO-d₆) δ ppm 8.96 (d, 1H), 7.88 (s, 1H), 7.70 (d, 2H), 7.48-7.50 (m, 1H), 7.29-7.33 (m, 2H), 7.25 (d, 1H), 7.17 (d, 2H), 6.94-7.00 (m, 3H), 6.18 (d, 1H), 3.66 (s, 2H), 2.52 (s, 3H), 2.30 (s, 3H), 2.23 (s, 3H), 2.15 (s, 3H).

### Example 10

### N-((4-chloro-2-methylphenyl)(phenyl)methyl)-2-(2-(pyrimidin-5-yl)benzofuran-5-yl)acetamide

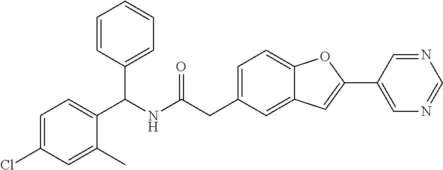 [SMILES: Cc1cc(Cl)ccc1C(NC(=O)Cc1ccc2oc(-c3cncnc3)cc2c1)c1ccccc1]

### a) 2-(2-bromobenzofuran-5-yl)acetic acid

The title compound was prepared from methyl 2-(2-bromobenzofuran-5-yl)acetate following essentially the procedure of Example 1(f). LCMS-P1: 255/257 [M+H]⁺; Rₜ: 1.115 min.

### b) 2-(2-bromobenzofuran-5-yl)-N-((4-chloro-2-methylphenyl)(phenyl)methyl)acetamide

To a stirred solution of 2-(2-bromobenzofuran-5-yl)acetic acid (177 mg, 0.7 mmol) in CH₂Cl₂ (20 mL) was added HOBt (108 mg, 0.8 mmol), EDC (154 mg, 0.8 mmol), DIPEA (271 mg, 2.1 mmol) and (4-chloro-2-methylphenyl)(phenyl)methanamine (223 mg, 0.8 mmol). The reaction mixture was stirred at rt overnight. The reaction mixture was washed with dilute HCl (3×10 mL) and brine (3×10 mL), dried over Na₂SO₄, and concentrated. The resultant residue was purified by column chromatography (20% EtOAc/petroleum ether) to obtain the title compound (216 mg, 66%) as a white solid. LCMS-P1: 468/470 [M+H]⁺; Rₜ: 1.853 min.

### c) N-((4-chloro-2-methylphenyl)(phenyl)methyl)-2-(2-(pyrimidin-5-yl)benzofuran-5-yl)acetamide

To a solution of 2-(2-bromobenzofuran-5-yl)-N-((4-chloro-2-methylphenyl)(phenyl)methyl)acetamide (60 mg, 0.13 mmol) in 1,4-dioxane (1 mL) and 1 drop water was added pyrimidin-5-ylboronic acid (24 mg, 0.19 mmol), Pd(dppf)Cl₂ (29 mg, 0.04 mmol), and K₂CO₃ (36 mg, 0.26 mmol) under N₂. The reaction vessel was sealed and heated under microwave irradiation at 100° C. for 30 min. After cooling to rt, water (10 mL) was added and the reaction mixture was extracted with EtOAc (3×10 mL). The combined organic layers were washed with brine (3×10 mL), dried over Na₂SO₄, and concentrated. The resultant residue was purified by column chromatography (5:1 petroleum ether/EtOAc) to obtain the title compound (25 mg, 42%) as a white solid. LCMS-P1: 468 [M+H]⁺; Rₜ: 1.722 min. ¹H NMR (500 MHz, CDCl₃) δ ppm 9.20 (d, J=6.0 Hz, 3H), 7.57-7.55 (m, 2H), 7.29-7.25 (m, 3H), 7.18-7.15 (m, 2H), 7.10-6.84 (m, 3H), 6.35 (d, J=8.0 Hz, 1H), 6.87 (d, J=8.0 Hz, 1H), 5.96 (d, J=8.0 Hz, 1H), 3.75 (s, 2H), 2.05 (s, 3H).

### Example 11

### methyl 3-(5-(2-(((2,4-dimethylphenyl)(phenyl)methyl)amino)-2-oxoethyl)benzofuran-2-yl)picolinate

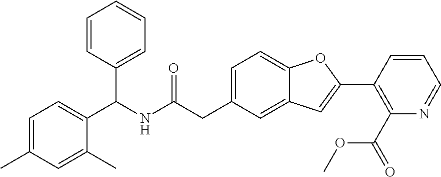 [SMILES: COC(=O)c1ncccc1-c1cc2cc(CC(=O)NC(c3ccccc3)c3ccc(C)cc3C)ccc2o1]

### a) benzyl 2-(2-bromobenzofuran-5-yl)acetate

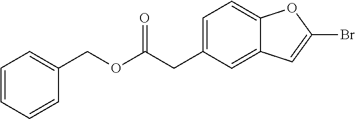 [SMILES: O=C(Cc1ccc2oc(Br)cc2c1)OCc1ccccc1]

To a solution of 2-(2-bromobenzofuran-5-yl)acetic acid (2.53 g, 9.92 mmol) and benzyl bromide (1.42 mL, 11.9 mmol) in DMF (60 mL) was added K₂CO₃ (2.74 g, 19.84 mmol), and the reaction mixture was stirred at rt for 1 h. The reaction mixture was diluted with water (100 mL), extracted with EtOAc (3×60 mL), and washed with brine (3×60 mL). The combined organic layers were dried over anhydrous Na₂SO₄, filtered, and concentrated. The resultant residue was recrystallized with CH₂Cl₂ and petroleum ether to obtain the title compound (3.08 g, 90%) as a white crystalline solid. LCMS-P1: 368/370 [M+Na]⁺; Rₜ: 2.06 min.

### b) (5-(2-(benzyloxy)-2-oxoethyl)benzofuran-2-yl)boronic acid

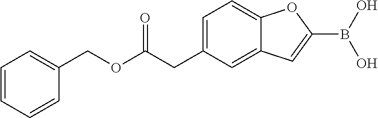 [SMILES: O=C(Cc1ccc2oc(B(O)O)cc2c1)OCc1ccccc1]

To a solution of benzyl 2-(2-bromobenzofuran-5-yl)acetate (2 g, 5.8 mmol) in dry THF (30 mL) at 0° C. under N₂ was added i-PrMgCl (3.8 mL, 7.5 mmol, 2 N in THF). The reaction mixture was stirred at 0° C. for 1 h. Triisopropylborate (1.8 mL, 7.5 mmol) was added in one portion and the reaction mixture was stirred for an additional 4 h. Saturated NH₄Cl (40 mL) was added and the reaction mixture was extracted with EtOAc (3×40 mL). The combined organic layers were washed with brine (3×30 mL) and dried over Na₂SO₄. The solvent was evaporated to obtain the title compound (1.69 g, 41%) as a yellow solid. LCMS-P1: 333.0 [M+Na]⁺; Rₜ: 1.75 min.

### c) methyl 3-(5-(2-(benzyloxy)-2-oxoethyl)benzofuran-2-yl)picolinate

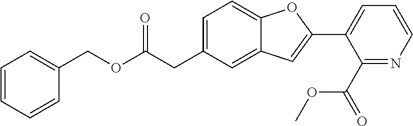 [SMILES: COC(=O)c1ncccc1-c1cc2cc(CC(=O)OCc3ccccc3)ccc2o1]

To a solution of (5-(2-(benzyloxy)-2-oxoethyl)benzofuran-2-yl)boronic acid (832 mg, 2.68 mmol) and methyl 3-bromopicolinate (579 mg, 2.68 mmol) in 1,4-dioxane (10 mL) and several drops of water were added Pd(dppf)Cl₂ (196 mg, 0.27 mmol) and K₂CO₃ (739 mg, 5.36 mmol). The reaction mixture was heated under microwave irradiation at 100° C. for 30 min. After filtration, water was added, and the reaction mixture was extracted with EtOAc (3×20 mL). After the solvent was removed, the resultant residue was purified by column chromatography (5:1 petroleum ether/EtOAc) to obtain the title compound (148 mg, 83%) as a yellow oil. LCMS-P1: 402.0 [M+H]⁺; Rₜ: 1.86 min.

### d) 2-(2-(2-(methoxycarbonyl)pyridin-3-yl)benzofuran-5-yl)acetic acid

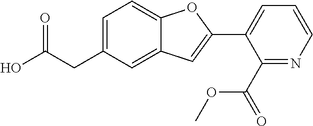 [SMILES: COC(=O)c1ncccc1-c1cc2cc(CC(=O)O)ccc2o1]

To a solution of methyl 3-(5-(2-(benzyloxy)-2-oxoethyl)benzofuran-2-yl)picolinate (75 mg, 0.19 mmol) in EtOH (10 mL) was added 10% Pd/C (6 mg). The reaction mixture was stirred at rt under H₂ atmosphere for 1 h, followed by filtration. The filtrate was concentrated to obtain the title compound (69 mg, 82%) as pale yellow solid. LCMS-P1: 312.0 [M+H]⁺; Rₜ: 1.41 min.

### e) methyl 3-(5-(2-(((2,4-dimethylphenyl)(phenyl)methyl)amino)-2-oxoethyl)benzofuran-2-yl)picolinate

The title compound was synthesized from 2-(2-(2-(methoxycarbonyl)pyridin-3-yl)benzofuran-5-yl)acetic acid and (2,4-dimethylphenyl)(phenyl)methanamine hydrochloride following essentially the procedure of Example 2(b) (54 mg, 49%). The title compound was obtained as a white solid. LCMS-P1: 505.0 [M+H]⁺; Rₜ: 1.8 min. ¹H NMR (400 MHz, CDCl₃) δ ppm 8.66 (d, J=5.6 Hz, 1H), 8.19 (dd, J=8.4, 1.6 Hz, 1H), 7.53 (t, J=6.4 Hz, 2H), 7.48 (d, J=8.4 Hz, 1H), 7.21-7.24 (m, 4H), 7.07 (d, J=8.0 Hz, 2H), 7.03 (s, 1H), 6.97 (s, 1H) 6.91 (d, J=7.2 Hz, 1H), 6.77 (d, J=8.4 Hz, 1H), 6.38 (d, J=8.4 Hz, 1H), 5.92 (d, J=8.4 Hz, 1H), 3.98 (s, 3H), 3.73 (s, 2H), 2.28 (s, 3H), 2.21 (s, 3H).

### Example 12

### N-((4-chloro-2-methylphenyl)(phenyl)methyl)-2-(2-(2-(hydroxymethyl)pyridin-3-yl)benzofuran-5-yl)acetamide

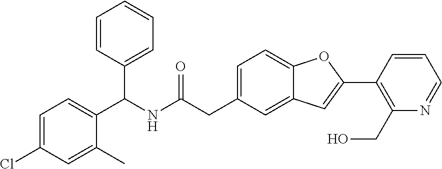 [SMILES: Cc1cc(Cl)ccc1C(NC(=O)Cc1ccc2oc(-c3cccnc3CO)cc2c1)c1ccccc1]

### a) (3-bromopyridin-2-yl)methanol

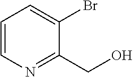

To a solution of methyl 3-bromopicolinate (1 g, 4.6 mmol) in MeOH (50 mL) at 0° C. was added NaBH₄ (883 mg, 23.2 mmol). The reaction mixture was stirred at rt overnight, followed by concentration under reduced pressure. The resultant residue was dissolved in EtOAc (50 mL), washed with aq. NH₄Cl (3×20 mL), dried over Na₂SO₄, filtered, and concentrated to afford the title compound (101 g, 92%) as a white solid. LCMS-P1: 190/192 [M+H]⁺; Rₜ: 1.070 min.

### b) (5-(2-methoxy-2-oxoethyl)benzofuran-2-yl)boronic acid

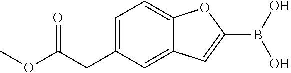 [SMILES: COC(=O)Cc1ccc2oc(B(O)O)cc2c1]

To a solution of methyl 2-(2-bromobenzofuran-5-yl)acetate (268 mg, 1.0 mmol) in THF (15 mL) was added i-PrMgCl (133 mg, 1.3 mmol) under N₂ at 0° C. The reaction mixture was stirred for 0.5 h. Triisopropylborate (245 mg, 1.3 mmol) was added and the reaction mixture was allowed to stir at rt overnight. Water (15 mL) was added and the reaction mixture was extracted with EtOAc (3×10 mL). The organic layer was dried over Na₂SO₄, filtered, and concentrated to obtain the title compound (150 mg, 65%) as a yellow solid. LCMS-P1: 235.0 [M+H]⁺; Rₜ: 1.281 min.

### c) methyl 2-(2-(2-(hydroxymethyl)pyridin-3-yl)benzofuran-5-yl)acetate

The title compound was synthesized from (5-(2-methoxy-2-oxoethyl)benzofuran-2-yl)boronic acid and (3-bromopyridin-2-yl)methanol following essentially the procedure of Example 11(c) and purified by preparative TLC with 1:2 EtOAc/CH₂Cl₂ (40 mg, 71%) as pale yellow solid. LCMS-P1: 298.0 [M+H]⁺; Rₜ: 1.252 min.

### d) 2-(2-(2-(hydroxymethyl)pyridin-3-yl)benzofuran-5-yl)acetic acid

To a solution of methyl 2-(2-(2-(hydroxymethyl)pyridin-3-yl)benzofuran-5-yl)acetate (40 mg, 0.13 mmol) in THF (8 mL) and water (2 mL) was added LiOH (28 mg, 0.67 mmol). The reaction mixture was stirred at rt overnight. Water (10 mL) was added and then 1 N HCl was used to adjust the pH value of the aqueous phase to 3-4. The organic layer was evaporated to afford the title compound (35 mg, 81%) as a yellow solid. LCMS-P1: 284.0 [M+H]⁺; Rₜ: 1.114 min.

### e) N-((4-chloro-2-methylphenyl)(phenyl)methyl)-2-(2-(2-(hydroxymethyl)pyridin-3-yl)benzofuran-5-yl)acetamide

To a stirred solution of 2-(2-(2-(hydroxymethyl)pyridin-3-yl)benzofuran-5-yl)acetic acid (35 mg, 0.09 mmol) in DMF (5 mL) were added HOBt (15 mg, 0.11 mmol), EDC (29 mg, 0.11 mmol), DIPEA (47 mg, 0.36 mmol), and (4-chloro-2-methylphenyl)(phenyl)methanamine hydrochloride (29 mg, 0.11 mmol). The reaction mixture was stirred at rt overnight. The reaction mixture was washed with diluted HCl (3×10 mL), brine (3×10 mL), dried over Na₂SO₄, and concentrated. The resultant residue was purified by preparative HPLC to obtain the title compound (5 mg, 11%) as a white solid. LCMS-P1: 497.0 [M+H]⁺; Rₜ: 1.544 min. ¹H NMR (400 MHz, DMSO-d₆) δ ppm 9.00 (d, J=8.4 Hz, 1H), 8.60 (s, 1H), 8.30 (d, J=7.6 Hz, 1H), 7.58-7.53 (m, 3H), 7.44 (s, 1H), 7.35-7.22 (m, 6H), 7.19 (d, J=7.2 Hz, 2H), 7.13 (d, J=8.4 Hz, 1H), 6.20 (d, J=8.4 Hz, 1H), 4.81 (s, 2H), 3.62 (s, 2H), 2.18 (s, 3H).

### Example 13

### 2-(2-((3,5-dimethylisoxazol-4-yl)(ethoxy)methyl)benzofuran-5-yl)-N-((2,4-dimethylphenyl)(phenyl)methyl)acetamide

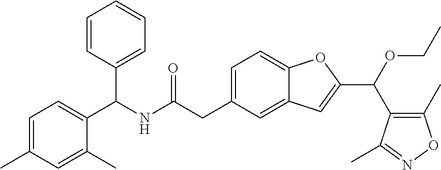 [SMILES: CCOC(c1cc2cc(CC(=O)NC(c3ccccc3)c3ccc(C)cc3C)ccc2o1)c1c(C)noc1C]

### a) methyl 2-(2-((3,5-dimethylisoxazol-4-yl)(hydroxy)methyl)benzofuran-5-yl)acetate

To a solution of methyl 2-(2-bromobenzofuran-5-yl)acetate (2.0 mg, 7.46 mmol) in THF (50 mL) at 0° C. was added i-PrMgCl (5.6 mL, 11.2 mmol, 2 N in THF). The reaction mixture was stirred at 0° C. for 30 min. 3,5-dimethylisoxazole-4-carbaldehyde (1.5 g, 12 mmol) was added and the reaction mixture was stirred for 2 h. Saturated NH₄Cl (10 mL) was added and the reaction mixture was extracted with EtOAc (3×50 mL). The combined organic layers were washed with brine (3×15 mL), dried over Na₂SO₄, filtered, and concentrated. The resultant residue was purified by column chromatography (30% EtOAc/petroleum ether) to afford the title compound (900 mg, 38%) as a yellow oil. LCMS-P1: 316 [M+H]⁺; Rₜ: 1.481 min.

### b) 2-(2-((3,5-dimethylisoxazol-4-yl)(hydroxy)methyl)benzofuran-5-yl)acetic acid

To a solution of methyl 2-(24(3,5-dimethylisoxazol-4-yl)(hydroxy)methyl)benzofuran-5-yl)acetate (900 mg, 2.86 mmol) in THF (20 mL) and water (10 mL) was added LiOH (600.6 mg, 14.3 mmol). The reaction mixture was stirred at rt for 1 h and then heated to 40° C. for 2 h. Water (10 mL) was added, and AcOH was used to adjust the aqueous phase to pH=6-7. The reaction mixture was extracted with EtOAc (3×50 mL). The combined organic layers were washed with brine (3×15 mL), dried over Na₂SO₄, filtered, and concentrated to afford the title compound (650 mg, 75.6%) as a yellowish solid. LCMS-P1: 302 [M+H]⁺; Rₜ: 1.277 min.

### c) 2-(2-((3,5-dimethylisoxazol-4-yl)(hydroxy)methyl)benzofuran-5-yl)-N-((2,4-dimethylphenyl)(phenyl)methyl)acetamide

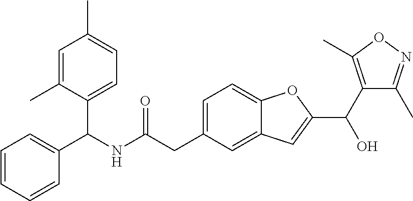 [SMILES: Cc1ccc(C(NC(=O)Cc2ccc3oc(C(O)c4c(C)noc4C)cc3c2)c2ccccc2)c(C)c1]

The title compound was synthesized from 2-(2-((3,5-dimethylisoxazol-4-yl)(hydroxy)methyl)benzofuran-5-yl)acetic acid and (2,4-dimethylphenyl)(phenyl)methanamine hydrochloride following essentially the procedure of Example 10(b) (805 mg, 75%) as a white solid. LCMS-P1: 495 [M+H]⁺; Rₜ: 1.688 min. ¹H NMR (400 MHz, CDCl₃) δ ppm 7.34-7.31 (m, 2H), 7.19-7.09 (m, 4H), 6.97 (d, J=7.2 Hz, 2H), 6.89 (s, 1H), 6.81 (d, J=8.0 Hz, 1H), 6.67 (d, J=8.0 Hz, 1H), 6.46 (d, J=8.0 Hz, 1H), 6.46 (s, 1H), 6.28 (d, J=8.0 Hz, 1H), 5.90 (d, J=8.0 Hz, 1H), 5.77 (s, 1H), 3.61 (s, 2H), 2.82 (br s, 1H), 2.30 (s, 3H), 2.20 (s, 3H), 2.16 (s, 3H), 2.12 (s, 3H).

### d) 2-(2-((3,5-dimethylisoxazol-4-yl)(ethoxy)methyl)benzofuran-5-yl)-N-((2,4-dimethylphenyl)(phenyl)methyl)acetamide

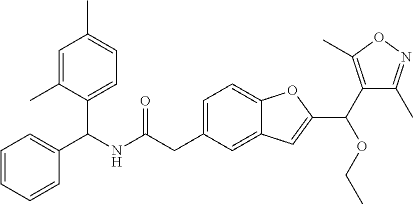 [SMILES: CCOC(c1cc2cc(CC(=O)NC(c3ccccc3)c3ccc(C)cc3C)ccc2o1)c1c(C)noc1C]

To a solution of 2-(2-((3,5-dimethylisoxazol-4-yl)(hydroxy)methyl)benzofuran-5-yl)-N-((2,4-dimethylphenyl)(phenyl)methyl)acetamide (20 mg, 0.04 mmol) in CH₂Cl₂ (5 mL) were added TFA (22.8 mg) and Et₃SiH (23.2 mg) at rt and the reaction mixture was stirred for 16 h. The reaction mixture was quenched by water (5 mL), extracted with CH₂Cl₂, washed with water, dried over Na₂SO₄, filtered, and concentrated. The resultant residue was purified by preparative HPLC to obtain the title compound (5 mg, 25%). LCMS-P1: 523 [M+H]⁺; Rₜ: 1.857 min. ¹H NMR (500 MHz, CDCl₃) δ ppm 7.42-7.39 (m, 2H), 7.24-7.12 (m, 4H), 7.03 (d, J=7.5 Hz, 2H), 6.96 (s, 1H), 6.88 (d, J=8.0 Hz, 1H), 6.72 (d, J=7.5 Hz, 1H), 6.56 (s, 1H), 6.35 (d, J=6.8 Hz, 1H), 5.91 (d, J=6.4 Hz, 1H), 5.41 (s, 1H), 3.70 (s, 2H), 3.61-3.57 (m, 2H), 2.40 (s, 3H), 2.27 (s, 3H), 2.25 (s, 3H), 2.19 (s, 3H), 1.30-1.25 (m, 3H).

### Example 14

### 2-(2-((3,5-dimethylisoxazol-4-yl)methyl)benzofuran-5-yl)-N-((2,4-dimethylphenyl)(phenyl)methyl)acetamide

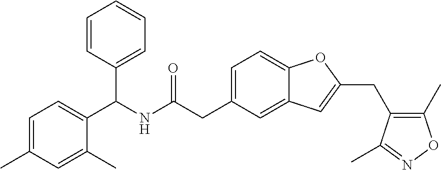

The title compound was obtained as a byproduct of Example 13(d) (7 mg, 37%). LCMS-P1: 479 [M+H]⁺; Rₜ: 1.803 min. ¹H NMR (400 MHz, CDCl₃) δ ppm 7.37-7.20 (m, 5H), 7.14-6.72 (m, 6H), 6.37-5.91 (m, 3H), 3.79 (s, 2H), 3.69 (s, 2H), 2.37 (s, 3H), 2.27 (s, 3H), 2.22 (s, 3H), 2.19 (s, 3H).

### Example 15

### N-((4-chloro-2-methylphenyl)(phenyl)methyl)-2-(2-((2-methylpyridin-3-yl)methyl)benzofuran-5-yl)acetamide

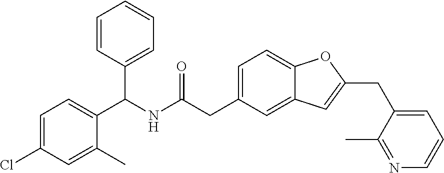 [SMILES: Cc1cc(Cl)ccc1C(NC(=O)Cc1ccc2oc(Cc3cccnc3C)cc2c1)c1ccccc1]

### a) 2-methylnicotinaldehyde

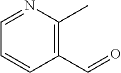

To a stirred solution of (2-methylpyridin-3-yl)methanol (200 mg, 1.6 mmol) in CH₂Cl₂ (10 mL) was added MnO₂ (200 mg, 2.3 mmol). The reaction mixture was refluxed overnight under N₂. The solid was removed by filtration and the filtrate was concentrated. The resultant residue was purified by flash chromatography (50% EtOAc/petroleum ether) to afford the title compound (120 mg, 60%) as a colorless liquid. LCMS-P1: 122.0 [M+H]⁺; Rₜ: 0.344 min.

### b) methyl 2-(2-(hydroxy(2-methylpyridin-3-yl)methyl)benzofuran-5-yl)acetate

To a solution of methyl 2-(2-bromobenzofuran-5-yl)acetate (0.35 g, 1.3 mmol) in dry THF (5 mL) at 0° C. was added i-PrMgCl (1.3 M solution in THF) (1.3 mL, 1.6 mmol) dropwise and the reaction mixture was stirred for 30 min. To the reaction mixture was added a solution of 2-methylnicotinaldehyde (157 mg, 1.3 mmol) in dry THF (5 mL) and the reaction mixture was stirred for an additional 2 h at 0° C. The reaction mixture was quenched with saturated NH₄Cl and extracted with EtOAc. The organic layer was dried over anhydrous Na₂SO₄, filtered, and concentrated under reduced pressure. The resultant residue was purified by flash column chromatography (5-10% MeOH/CH₂Cl₂) to afford the title compound (160 mg, 40%) as a yellow viscous liquid. TLC: 100% EtOAc, R_{f}=0.2. ¹H NMR (300 MHz, MeOD-d₄) δ ppm 8.35 (dd, J=4.9, 1.6 Hz, 1H), 8.00 (dd, J=7.9, 1.5 Hz, 1H), 7.44 (d, J=1.3 Hz, 1H), 7.37-7.30 (m, 2H), 7.16 (dd, J=8.4, 1.6 Hz, 1H), 6.56 (s, 1H), 6.08 (s, 1H), 3.69 (s, 2H), 3.65 (s, 3H), 2.52 (s, 3H).

### c) methyl 2-(2-(chloro(2-methylpyridin-3-yl)methyl)benzofuran-5-yl)acetate

A stirred solution of methyl 2-(2-(hydroxy(2-methylpyridin-3-yl)methyl)benzofuran-5-yl)acetate (100 mg, 0.032 mmol) in SOCl₂ (5 mL) was heated to 50° C. under N₂ for 1 h. After cooling to rt, SOCl₂ was removed under reduced pressure to afford the title compound.

### d) methyl 2-(2-((2-methylpyridin-3-yl)methyl)benzofuran-5-yl)acetate

Zn (125 mg, 1.9 mmol) was added to a solution of methyl 2-(2-(chloro(2-methylpyridin-3-yl)methyl)benzofuran-5-yl)acetate (freshly prepared) in AcOH (10 mL). The reaction mixture was stirred at 30° C. overnight. Zn was removed by filtration, and AcOH was removed under reduced pressure. The resultant residue was neutralized with saturated aq. NaHCO₃ and extracted with EtOAc (3×30 mL). The combined organic layers were washed with brine, dried over Na₂SO₄, filtered, and concentrated. The resultant residue was purified by flash chromatography (33-50% EtOAc/petroleum ether) to afford the title compound (40 mg, 42%). LCMS-P1: 296.0 [M+H]⁺; Rₜ: 1.346 min.

### e) 2-(2-((2-methylpyridin-3-yl)methyl)benzofuran-5-yl)acetic acid

To a stirred solution of methyl 2-(2-((2-methylpyridin-3-yl)methyl)benzofuran-5-yl)acetate (40 mg, 0.14 mmol) in THF (4 mL) and H₂O (1 mL) was added NaOH (16 mg, 0.42 mmol). The reaction mixture was stirred overnight at 50° C. After cooling to rt, 1 N aq. HCl (0.5 mL) was added. All solvent was removed under reduced pressure to afford the title compound (60 mg). LCMS-P1: 282.0 [M+H]⁺; Rₜ: 1.173 min.

### f) N-((4-chloro-2-methylphenyl)(phenyl)methyl)-2-(2-((2-methylpyridin-3-yl)methyl)benzofuran-5-yl)acetamide

To a stirred solution of 2-(2-((2-methylpyridin-3-yl)methyl)benzofuran-5-yl)acetic acid (60 mg) in DMF (4 mL) was added HOBt (30 mg, 0.21 mmol), EDC (40 mg, 0.21 mmol), DIPEA (1 mL), and (4-chloro-2-methylphenyl)(phenyl)methanamine (38 mg, 0.21 mmol). The reaction mixture was stirred at 45° C. overnight. After cooling to rt, the reaction mixture was extracted with EtOAc (3×15 mL). The combined organic layers were washed with brine (2×15 mL), dried over Na₂SO₄, and concentrated. The resultant residue was purified by column chromatography (25-50% EtOAc/petroleum ether) to obtain the title compound (20 mg, 30% over two steps) as a white solid. LCMS-P1: 495.0 [M+H]⁺; Rₜ: 1.517 min. ¹H NMR (400 MHz, CDCl₃) δ ppm 8.36 (d, J=4 Hz, 1H), 7.43 (d, J=8.0 Hz, 1H), 7.32-6.93 (m, 12H), 6.74 (d, J=8.0 Hz, 1H), 6.25 (d, J=8.0 Hz, 1H), 6.21 (s, 1H), 5.85 (d, J=8.0 Hz, 1H), 4.03 (s, 2H), 3.62 (s, 2H), 2.53 (s, 3H), 2.10 (s, 3H).

### Example 16

### N-((2,4-dimethylphenyl)(phenyl)methyl)-2-(2-((2-methylpyridin-3-yl)methyl)benzofuran-5-yl)acetamide

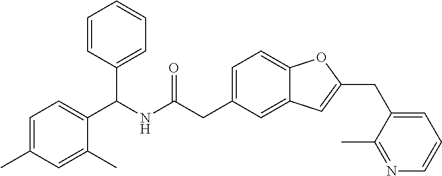 [SMILES: Cc1ccc(C(NC(=O)Cc2ccc3oc(Cc4cccnc4C)cc3c2)c2ccccc2)c(C)c1]

To the solution of 2-(2((2-methylpyridin-3-yl)methyl)benzofuran-5-yl)acetic acid in DMF (3 mL) was added (2,4-dimethylphenyl)(phenyl)methanamine (44 mg, 0.18 mmol), EDC (34 mg, 0.18 mmol), HOBt (24 mg, 0.18 mmol), and DIPEA (38 mg, 0.3 mmol). The reaction mixture was heated at 45° C. overnight. After cooling to rt, the reaction mixture was poured into water and extracted with EtOAc (2×5 mL). The combined organic layers were washed with brine, dried over Na₂SO₄, filtered, and concentrated. The resultant residue was purified by preparative TLC to afford the title compound (11 mg, 15.5% over two steps) as a white solid. LCMS-P1: 475 [M+H]⁺. ¹H NMR (400 MHz, DMSO-d₆) δ ppm 8.85 (d, J=8.4 Hz, 1H), 8.35 (d, J=4.8 Hz, 1H), 7.57 (d, J=7.6 Hz, 1H), 7.39 (d, J=9.6 Hz, 2H), 7.30 (t, J=7.2 Hz, 2H), 7.24-7.12 (m, 5H), 6.99-6.92 (m, 3H), 6.52 (s, 1H), 6.15 (d, J=8.4 Hz, 1H), 4.17 (s, 2H), 3.56 (s, 2H), 2.50 (s, 3H), 2.22 (s 3H), 2.14 (s 3H).

### Example 17

### 2-(2-(3,5-dimethylisoxazol-4-yl)benzo[d]oxazol-5-yl)-N-((2,4-dimethylphenyl)(phenyl)methyl)acetamide

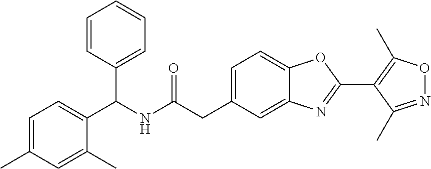 [SMILES: Cc1ccc(C(NC(=O)Cc2ccc3oc(-c4c(C)noc4C)nc3c2)c2ccccc2)c(C)c1]

### a) 2-(4-hydroxy-3-nitrophenyl)acetic acid

To a solution of 2-(4-hydroxyphenyl)acetic acid (10 g, 65.7 mmol) in AcOH (100 mL) at 0° C. was added concentrated HNO₃ (5.3 mL, 118.4 mmol) dropwise and the reaction mixture was stirred for 1 h. The yellow solution was allowed to warm to rt. The resultant precipitate was collected by filtration and recrystallized from EtOAc/hexanes to provide the title compound (5.0 g, 39%) as a yellow solid. ¹H NMR (400 MHz, DMSO-d₆) δ ppm 12.44 (br s, 1H), 10.87 (s, 1H), 7.80 (d, 1H), 7.44 (dd, 1H), 7.08 (d, 1H), 3.59 (s, 2H).

### b) methyl 2-(4-hydroxy-3-nitrophenyl)acetate

To a solution of 2-(4-hydroxy-3-nitrophenyl)acetic acid (5.0 g, 25.38 mmol) in MeOH (15 mL) was added concentrated H₂SO₄ (1 mL) and the reaction mixture was refluxed overnight at 70° C. The reaction mixture was cooled to rt and then concentrated. The resultant residue was neutralized with saturated aq. NaHCO₃. The resultant precipitate was collected by filtration, washed with water, and dried under vacuum to provide the title compound (5.0 g, 93%). ¹H NMR (400 MHz, DMSO-d₆) δ ppm 10.91 (s, 1H), 7.82 (d, 1H), 7.45 (dd, 1H), 7.09 (d, 1H), 3.71 (s, 2H), 3.62 (s, 3H).

### c) methyl 2-(3-amino-4-hydroxyphenyl)acetate

To a solution of methyl 2-(4-hydroxy-3-nitrophenyl)acetate (4.5 g, 21.3 mmol) in a mixture of MeOH (15 mL) and THF (15 mL) was added 10% Pd/C (0.900 g) and the reaction mixture was stirred at rt for 2 h under 5 kg of H₂ pressure. The reaction mixture was filtered through Celite®, washed through with MeOH, and concentrated. The resultant residue was purified by column chromatography (2% MeOH/CH₂Cl₂) to obtain the title compound (2.8 g, 65%). ¹H NMR (400 MHz, DMSO-d₆) δ ppm 8.92 (s, 1H), 6.55 (d, 1H), 6.47 (d, 1H), 6.26 (dd, 1H), 4.51 (s, 2H), 3.57 (s, 3H), 3.38 (s, 2H).

### d) methyl 2-(3-(3,5-dimethylisoxazole-4-carboxamido)-4-hydroxyphenyl)acetate

To a solution of 3,5-dimethylisoxazole-4-carboxylic acid (2.4 g, 1.70 mmol) in CH₂Cl₂ (15 mL), and DMF (0.5 mL) at 0° C. was added (COCl)₂ (1.76 mL, 2.04 mmol) and the reaction mixture was stirred for 1 h. Following concentration, the residue was dissolved in CH₂Cl₂ (15 mL) and Et₃N (8.3 mL, 6.01 mmol) and methyl 2-(3-amino-4-hydroxyphenyl)acetate were added. The reaction mixture was stirred at rt overnight. The reaction mixture was poured into water (150 mL), extracted with EtOAc (350 mL), dried over Na₂SO₄, filtered, and concentrated to provide the title compound (5.0 g). e) methyl 2-(2-(3,5-dimethylisoxazol-4-yl)benzo[d]oxazol-5-yl)acetate

To a solution of methyl 2-(3-(3,5-dimethylisoxazole-4-carboxamido)-4-hydroxyphenyl)acetate (5.0 g) in toluene (20 mL) was added p-TsOH (4.37 g, 2.29 mmol). The reaction mixture was refluxed for 4 h and then stirred at rt overnight. The reaction mixture was poured into water (150 mL), extracted with EtOAc (350 mL), dried over Na₂SO₄, filtered, and concentrated. The resultant residue was purified by column chromatography (14% EtOAc/hexanes) to obtain the title compound (0.9 g). ¹H NMR (400 MHz, DMSO-d₆) δ ppm 7.72 (s, 1H), 7.69 (d, 2H), 7.31 (dd, 1H), 3.83 (s, 2H), 3.63 (s, 3H), 2.81 (s, 3H), 2.56 (s, 3H).

### f) 2-(2-(3,5-dimethylisoxazol-4-yl)benzo[d]oxazol-5-yl)acetic acid

To a solution of methyl 2-(2-(3,5-dimethylisoxazol-4-yl)benzo[d]oxazol-5-yl)acetate (0.9 g, 3.14 mmol) in EtOH (2 mL) was added 4 N NaOH (30 mL) and the reaction mixture was stirred at 45° C. for 30 min. Following concentration, the resultant residue was acidified with 1 N HCl. The resultant precipitate was collected by filtration, dissolved in MeOH (15 mL), dried over Na₂SO₄, filtered, and concentrated to provide the title compound (0.734 g, 86%). ¹H NMR (400 MHz, DMSO-d₆) δ ppm 12.5 (br s, 1H), 7.68-7.72 (m, 2H), 7.31 (dd, 1H), 3.72 (s, 2H), 2.82 (s, 3H), 2.57 (s, 3H).

### g) 2-(2-(3,5-dimethylisoxazol-4-yl)benzo[d]oxazol-5-yl)-N-((2,4-dimethylphenyl)(phenyl)methyl)acetamide

To a solution of 2-(2-(3,5-dimethylisoxazol-4-yl)benzo[d]oxazol-5-yl)acetic acid (0.100 g, 0.36 mmol) in DMF (5 mL), EDC (0.085 g, 0.44 mmol), HOBt (0.067 g, 0.44 mmol), DMAP (0.090 g, 0.73 mmol), and (2,4-dimethylphenyl)(phenyl)methanamine (0.093 g, 0.44 mmol) were added, and the reaction mixture was stirred overnight at rt. Water (10 mL) was added dropwise. The resultant precipitate was collected by filtration, washed with hexanes (10 mL), and dried under vacuum to provide the title compound (0.080 g, 47%). LCMS-X¹: m/z=457.6 [M+H]⁺; Rₜ: 7.22 min. ¹H NMR (400 MHz, DMSO-d₆) δ ppm 8.93 (d, 1H), 7.66-7.71 (m, 2H), 7.32 (t, 3H), 7.24-7.29 (m, 1H), 7.18 (d, 2H), 6.96-7.00 (m, 3H), 6.19 (d, 1H), 3.66 (s, 2H), 2.82 (s, 3H), 2.57 (s, 3H), 2.32 (s, 3H), 2.15 (s, 3H).

### Example 18

### N-((4-chloro-2-methylphenyl)(phenyl)methyl)-2-(2-(2,4-dimethylpyridin-3-yl)benzo[d]oxazol-5-yl)acetamide

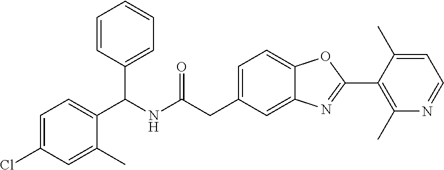 [SMILES: Cc1cc(Cl)ccc1C(NC(=O)Cc1ccc2oc(-c3c(C)ccnc3C)nc2c1)c1ccccc1]

### a) N-((4-chloro-2-methylphenyl)(phenyl)methyl)-2-(4-hydroxy-3-nitrophenyl)acetamide

The title compound was synthesized from 2-(4-hydroxy-3-nitrophenyl)acetic acid and (4-chloro-2-methylphenyl)(phenyl)methanamine following essentially the procedure of Example 2(b) (8.6 g, 69%). LCMS-P1: m/z 411 [M+H]⁺; Rₜ: 1.71 min.

### b) 2-(3-amino-4-hydroxyphenyl)-N-((4-chloro-2-methylphenyl)(phenyl)methyl)acetamide

To a solution of N-((4-chloro-2-methylphenyl)(phenyl)methyl)-2-(4-hydroxy-3-nitrophenyl)acetamide (6.0 g, 14.6 mmol) in aq. NH₄Cl (100 mL) were added Zn (4.75 g, 73 mmol) and EtOH (25 mL). The reaction mixture was stirred at 60° C. for 3 h. Upon cooling to rt, Zn was removed by filtration and the reaction mixture was extracted with EtOAc (3×150 mL), washed with brine, dried over Na₂SO₄, filtered, and concentrated. The resultant residue was purified by column chromatography (25% EtOAc/petroleum ether) to provide the title compound (5.0 g, 90%) as a brown solid. LCMS-P1: m/z 381.0.0 [M+H]⁺; Rₜ: 1.714 min. ¹H NMR (400 MHz, DMSO-d₆) δ ppm 2.17 (s, 3H), 3.25 (s, 2H), 4.45 (s, 2H), 6.16 (d, J=8.0 Hz, 1H), 6.26 (dd, J=10.4, 2.0 Hz, 1H), 6.50 (dd, J=8.0, 2.0 Hz, 2H), 7.23 (m, 8H), 8.77 (t, J=8.0 Hz, 2H).

### c) N-(5-(2-(((4-chloro-2-methylphenyl)(phenyl)methyl)amino)-2-oxoethyl)-2-hydroxyphenyl)-2,4-dimethylnicotinamide

To a solution of 2,4-dimethylnicotinic acid (151 mg, 1 mmol) in CH₂Cl₂ (30 mL) was added SOCl₂ (595 mg, 5 mmol). The reaction mixture was heated to reflux for 1 h. The solvent was removed under reduced pressure and the residue was diluted with CH₂Cl₂ (5 mL). The solution was added dropwise to another solution of 2-(3-amino-4-hydroxyphenyl)-N-((4-chloro-2-methylphenyl)(phenyl)methyl)acetamide (380 mg, 1 mmol) and DIPEA (258 mg, 2 mmol) in CH₂Cl₂ (50 mL) at rt. After stirring for 3 h, water (20 mL) was added. The reaction mixture was extracted with CH₂Cl₂ (3×60 mL), washed with water, dried over Na₂SO₄, filtered, and concentrated to afford the title compound (320 mg, 62%). LCMS-P1: m/z 514 [M+H]⁺; Rₜ: 1.525 min.

### d) N-((4-chloro-2-methylphenyl)(phenyl)methyl)-2-(2-(2,4-dimethylpyridin-3-yl)benzo[d]oxazol-5-yl)acetamide

To a solution of N-(5-(2-(((4-chloro-2-methylphenyl)(phenyl)methyl)amino)-2-oxoethyl)-2-hydroxyphenyl)-2,4-dimethylnicotinamide (320 mg, 0.62 mmol) in 1,4-dioxane (10 mL) was added p-TsOH (106.6 mg, 0.62 mmol). The reaction mixture was heated to reflux for 10 h. After cooling to rt, water (30 mL) was added. The reaction mixture was extracted with EtOAc (3×30 mL), washed with brine, dried over Na₂SO₄, filtered, and concentrated. The resultant residue was purified by preparative HPLC to afford the title compound (15 mg, 5%). LCMS-P1: 496 [M+H]⁺; Rₜ: 1.546 min. ¹H NMR (500 MHz, DMSO-d₆) δ ppm 9.06 (d, J=8.0 Hz, 1H), 8.53 (d, J=5.0 Hz, 1H), 7.74 (d, J=7.5 Hz, 2H), 7.40-6.20 (m, 10H), 6.21 (d, J=8.0 Hz, 1H), 3.71 (s, 2H), 2.46 (s, 3H), 2.31 (s, 3H), 2.21 (s, 3H).

### Example 19

### N-((4-chloro-2-methylphenyl)(phenyl)methyl)-2-methyl-2-(2-(2-methylpyridin-3-yl)benzo[d]oxazol-5-yl)propanamide

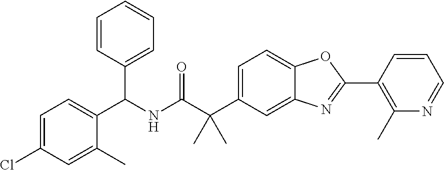 [SMILES: Cc1cc(Cl)ccc1C(NC(=O)C(C)(C)c1ccc2oc(-c3cccnc3C)nc2c1)c1ccccc1]

### a) methyl 2-(4-methoxy-3-nitrophenyl)acetate

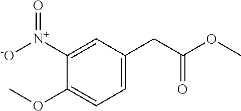

To a solution of methyl 2-(4-hydroxy-3-nitrophenyl)acetate (4.87 g, 23.1 mmol) at rt was added K₂CO₃ (8 g, 58 mmol), followed by slow addition of CH₃I (6.6 g, 46.5 mmol). The reaction mixture was stirred at rt overnight. The reaction mixture was then poured into ice-water (100 mL) and extracted with CH₂Cl₂ (3×300 mL). The combined organic layers were washed with brine, dried over Na₂SO₄, filtered, and concentrated to afford the title compound (5 g, 96%) as a yellow solid. LCMS-P1: 226.0 [M+H]⁺; Rₜ: 1.438 min.

### b) methyl 2-(4-methoxy-3-nitrophenyl)-2-methylpropanoate

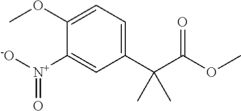

To a solution of methyl 2-(4-methoxy-3-nitrophenyl)acetate (4.6 g, 20.4 mmol) in THF (100 mL) at rt was added NaH (60%, 2.5 g, 62.5 mmol) slowly. The reaction mixture was stirred at rt for 2 h. CH₃I (14.5 g, 102.1 mmol) was added slowly at rt, and the reaction mixture was stirred at rt overnight. The reaction mixture was cooled to 0° C. and poured into saturated NH₄Cl (100 mL). The reaction mixture was extracted with CH₂Cl₂ (3×300 mL). The combined organic layers were washed with brine, dried over Na₂SO₄, filtered, and concentrated. The resultant residue was purified by column chromatography (1:10 EtOAc/petroleum ether) to provide the title compound (2 g, 39%). LCMS-P1: 254.0 [M+H]⁺; Rₜ: 1.58 min.

### c) 2-(4-hydroxy-3-nitrophenyl)-2-methylpropanoic acid

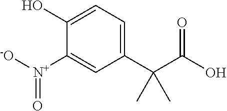

To a stirred solution of methyl 2-(4-methoxy-3-nitrophenyl)-2-methylpropanoate (1.2 g, 4.74 mmol) in CH₂Cl₂ (30 mL) at −40° C. was added a solution of BBr₃ in CH₂Cl₂ (5 mL) slowly, and the reaction mixture was allowed to warm to rt and stirred overnight. The reaction mixture was diluted with water (150 mL) and extracted with CH₂Cl₂ (3×300 mL). The combined organic layers were dried over Na₂SO₄, filtered, and concentrated. The resultant residue was purified by column chromatography (1:6 EtOAc/petroleum ether) to provide the title compound (580 mg, 54%). LCMS-P1: m/z 226 [M+H]⁺; Rₜ: 1.38 min.

### d) N-((4-chloro-2-methylphenyl)(phenyl)methyl)-2-(4-hydroxy-3-nitrophenyl)-2-methylpropanamide

The title compound was synthesized from 2-(4-hydroxy-3-nitrophenyl)-2-methylpropanoic acid and (4-chloro-2-methylphenyl)(phenyl)methanamine following essentially the procedure of Example 10(b) (1.0 g, 52%). LCMS-P1: m/z 439 [M+H]⁺; Rₜ: 1.85 min.

### e) 2-(3-amino-4-hydroxyphenyl)-N-((4-chloro-2-methylphenyl)(phenyl)methyl)-2-methylpropanamide

The title compound was synthesized from N-((4-chloro-2-methylphenyl)(phenyl)methyl)-2-(4-hydroxy-3-nitrophenyl)-2-methylpropanamide following essentially the procedure of Example 18(b).

### f) N-(5-(2-(((4-chloro-2-methylphenyl)(phenyl)methyl)amino)-2-oxoethyl)-2-hydroxyphenyl)-2-methylnicotinamide

To a solution of 2-methylnicotinic acid (110 mg, 0.8 mmol) in CH₂Cl₂ (10 mL) was added SOCl₂ (595 mg, 5 mmol). The reaction mixture was heated to reflux for 1 h. The solvent was removed under reduced pressure, and the residue was diluted with CH₂Cl₂ (5 mL). The solution was added dropwise to another solution of 2-(3-amino-4-hydroxyphenyl)-N-((4-chloro-2-methylphenyl)(phenyl)methyl)-2-methylpropanamide (250 mg, 0.6 mmol) and DIPEA (237 mg, 1.84 mmol) in CH₂Cl₂ (10 mL) at rt. After stirring for 3 h, water (20 mL) was added and the reaction mixture was extracted with CH₂Cl₂ (3×60 mL). The combined organic layers were washed with water, dried over Na₂SO₄, filtered, and concentrated to afford the title compound (300 mg, 93%). LC-MS: m/z 528 [M+H]⁺; Rₜ: 2.07 min.

### g) N-((4-chloro-2-methylphenyl)(phenyl)methyl)-2-methyl-2-(2-(2-methylpyridin-3-yl)benzo[d]oxazol-5-yl)propanamide

The title compound was synthesized from N-(5-(2-(((4-chloro-2-methylphenyl)(phenyl)methyl)amino)-2-oxoethyl)-2-hydroxyphenyl)-2-methylnicotinamide following essentially the procedure of Example 18(d). LC-MS: 510 [M+H]⁺; Rₜ: 2.19 min. ¹H NMR (400 MHz, MeOD-d₄) δ ppm 8.60 (m, 2H), 7.81 (d, J=8 Hz, 1H), 7.67 (d, J=4 Hz, 1H), 7.49-7.53 (m, 1H), 7.41-7.44 (m, 1H), 7.18-7.30 (m, 4H), 7.05 (m, 3H), 6.89 (d, J=2 Hz, 1H), 6.38 (s, 1H) 3.08 (s, 3H), 2.46 (s, 3H), 2.36 (s, 3H), 1.68 (s, 6H).

### Example 20

### 2-(4-chloro-2-methylphenyl)-N-((2-(2-methylpyridin-3-yl)benzofuran-5-yl)methyl)-2-phenylacetamide

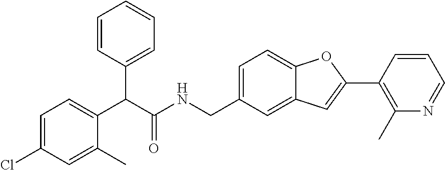 [SMILES: Cc1cc(Cl)ccc1C(C(=O)NCc1ccc2oc(-c3cccnc3C)cc2c1)c1ccccc1]

### a) (4-chloro-2-methylphenyl)(phenyl)methanol

To a solution of 1-bromo-4-chloro-2-methylbenzene (10.25 g, 50 mmol) in THF (300 mL) under Ar at −78° C. was added n-BuLi (22 mL, 2.5 N in hexanes) dropwise over 15 min. After 20 min, benzaldehyde (6.89 g, 65 mmol) was added. The reaction mixture was slowly warmed to rt, saturated NH₄Cl (15 mL) was added to quench the reaction, and the reaction mixture was extracted with EtOAc (3×150 mL). The combined organic layers were washed with brine (20 mL), dried over Na₂SO₄, filtered, and concentrated. The resultant residue was purified by column chromatography (40:1 petroleum ether/EtOAc) to obtain the title compound (8.0 g, 69%). LCMS-P1: 215 [M−OH]⁺; Rₜ: 1.67 min.

### b) 4-chloro-1-(chloro(phenyl)methyl)-2-methylbenzene

To a solution of (4-chloro-2-methylphenyl)(phenyl)methanol (2.32 g, 10 mmol) in CH₂Cl₂ (20 mL) at rt was added SOCl₂ (2.38 g). The reaction mixture was stirred for 3 h and then concentrated to afford the title compound (2.4 g) as a yellowish liquid (96%).

### c) 2-(4-chloro-2-methylphenyl)-2-phenylacetonitrile

A solution of 4-chloro-1-(chloro(phenyl)methyl)-2-methylbenzene (2.2 g, 8.8 mmol), K₂CO₃ (1.22 g, 8.8 mmol), and sodium cyanide (0.43 g, 8.8 mmol) in DMF (30 mL) was heated to 60° C. for 10 h. Water (60 mL) was added to the reaction mixture, and the reaction mixture was extracted with EtOAc (3×30 mL). The organic layers were washed with brine (3×30 mL), dried over Na₂SO₄, filtered, and concentrated to afford the title compound (2.4 g, 100%).

### d) 2-(4-chloro-2-methylphenyl)-2-phenylacetic acid

To a solution of 2-(4-chloro-2-methylphenyl)-2-phenylacetonitrile (400 mg, 1.65 mmol) in EtOH (12 mL) was added aq. NaOH (79 mg, 1.9 mmol), and the reaction mixture was refluxed at 100° C. for 7 h. The reaction mixture was concentrated under reduced pressure and diluted with water. The aqueous layer was acidified to pH 3 using 1.5 N HCl and extracted with EtOAc. The organic layer was dried over anhydrous Na₂SO₄, filtered, and concentrated under reduced pressure. The residue was dissolved in glacial AcOH (12 mL) to which was added conc. H₂SO₄ (12.5 mL) and water (12.5 mL). The reaction mixture was refluxed at 110° C. for 10 h. The reaction mixture was diluted with water and extracted with EtOAc. The organic layer was washed with water and brine, dried over anhydrous Na₂SO₄, filtered, and concentrated under reduced pressure to afford the title compound (200 mg, 80%). TLC: 20% EtOAc/petroleum ether, R_{f}=0.2. ¹H NMR (300 MHz, DMSO-d₆) δ ppm 12.88 (br s, 1H), 7.35-7.21 (m, 7H), 7.08 (d, J=8.1 Hz, 1H), 5.16 (s, 1H), 2.22 (s, 3H).

### e) 3-formyl-4-hydroxybenzonitrile

5-Bromosalicylaldehyde (20 g, 0.099 mol) was dissolved in dry DMF (80 mL) and CuCN (18.6 g, 0.21 mol) was added. The reaction mixture was heated to 150° C. under N₂ for 5 h. The reaction mixture was allowed to cool to rt. The reaction mixture was diluted with EtOAc and filtered through Celite®. The filtrate was diluted with EtOAc, washed with water and brine, dried over anhydrous Na₂SO₄, filtered, and concentrated. The resultant residue was purified by column chromatography (20-25% EtOAc/petroleum ether) to afford the title compound (3.0 g, 21%) as yellow solid. TLC: 20% EtOAc/petroleum ether, R_{f}=0.2. ¹H NMR (300 MHz, CDCl₃) δ ppm 11.47 (s, 1H), 9.93 (s, 1H), 7.94 (s, 1H), 7.78 (dd, J=8.7, 1.2 Hz, 1H), 7.12 (d, J=8.8 Hz, 1H).

### f) 3-(2,2-dibromovinyl)-4-hydroxybenzonitrile

To a solution of CCl₄ (12.8 g, 0.039 mol) in dry CH₂Cl₂ (30 mL) at 0° C. was added a solution of PPh₃ (32.0 g, 0.122 mol) in dry CH₂Cl₂ (30 mL) and reaction mixture was stirred for 15 min at 0° C. To the reaction mixture were added a solution of 3-formyl-4-hydroxybenzonitrile (3.0 g, 20.39 mmol) in dry CH₂Cl₂ (15 mL) followed by Et₃N (16.2 mL, 0.122 mol) dropwise and the reaction mixture was stirred at 0° C. for an additional 2 h. The reaction mixture was diluted with water and extracted with CH₂Cl₂. The organic layer was dried over anhydrous Na₂SO₄, filtered, and concentrated under reduced pressure. The resultant residue was purified by column chromatography (30% EtOAc/petroleum ether) to afford the title compound (0.4 g, 7%). TLC: 40% EtOAc/petroleum ether, R_{f}=0.4.

### g) 2-bromobenzofuran-5-carbonitrile

To a solution of 3-(2,2-dibromovinyl)-4-hydroxybenzonitrile (400 mg, 1.32 mmol) in dry THF (60 mL) was added K₃PO₄ (560 mg, 2.6 mmol). The reaction mixture was degassed with N₂ for 15 min. CuI (10 mg, 0.05 mmol) was added and the reaction mixture was stirred at 80′C for 3 h. The reaction mixture was diluted with water and extracted with CH₂Cl₂. The organic layer was dried over anhydrous Na₂SO₄, filtered, and concentrated under reduced pressure. The resultant residue was purified by column chromatography (6% EtOAc/petroleum ether) to afford the title compound (100 mg, 34%). TLC: 30% EtOAc/petroleum ether, R_{f}=0.5. ¹H NMR (300 MHz, DMSO-d₆) δ ppm 8.79 (s, 1H), 8.47-8.37 (m, 2H), 7.88 (s, 1H).

### h) 2-(2-methylpyridin-3-yl)benzofuran-5-carbonitrile

To a solution of 2-bromobenzofuran-5-carbonitrile (100 mg, 0.45 mmol) in toluene (3 mL) and EtOH (3 mL) was added 2-methylpyridine-3-boronic acid (74 mg, 0.54 mmol) and the solution was purged with Ar for 10 min. A solution of Na₂CO₃ (143 mg, 1.3 mmol) in water (8 mL) and catalytic Pd(dppf)Cl₂CH₂.Cl₂ (36 mg, 0.04 mmol) were added and the reaction mixture was heated to 80° C. for 3 h. The reaction mixture was cooled to rt and solvent was removed under reduced pressure. The resultant residue was diluted with EtOAc, washed with water and brine, dried over anhydrous Na₂SO₄, filtered, and concentrated under reduced pressure. The resultant residue was purified by column chromatography (25% EtOAc/petroleum ether) to afford the title compound (60 mg, 57%). TLC: 50% EtOAc/petroleum ether, R_{f}=0.3. ¹H NMR (400 MHz, CDCl₃) δ ppm 8.58 (dd, J=4.8, 1.6 Hz, 1H), 8.16 (dd, J=7.9, 1.7 Hz, 1H), 8.00 (m, 1H), 7.64 (m, 2H), 7.31 (dd, J=8.0, 4.8 Hz, 1H), 7.06 (s, 1H), 2.85 (s, 3H).

### i) (2-(2-methylpyridin-3-yl)benzofuran-5-yl)methanamine

To a solution of 2-(2-methylpyridin-3-yl)benzofuran-5-carbonitrile (75 mg, 0.32 mmol) in anhydrous THF (5 mL) was added LiAlH₄ (0.32 mL, 0.64 mmol, 2 M in THF) dropwise at 0° C. The reaction mixture was allowed to warm to rt and stirred for an additional 1.5 h. The reaction mixture was quenched carefully with water and diluted with EtOAc. The organic layer was washed with brine, dried over anhydrous Na₂SO₄, filtered, and concentrated under reduced pressure to afford the title compound (40 mg). TLC: 20% MeOH/CHCl₃, R_{f}=0.2. ¹H NMR (300 MHz, DMSO-d₆) δ ppm 8.49-8.47 (m, 1H), 8.19-8.15 (m, 1H), 7.63 (s, 1H), 7.55 (d, J=8.3 Hz, 1H), 7.38 (dd, J=8.0, 4.5 Hz, 1H), 7.33-7.29 (m, 2H), 3.80 (s, 2H), 2.75 (s, 3H).

### j) 2-(4-chloro-2-methylphenyl)-N-((2-(2-methylpyridin-3-yl)benzofuran-5-yl)methyl)-2-phenylacetamide

To a stirred solution of 2-(4-chloro-2-methylphenyl)-2-phenylacetic acid (43 mg, 0.16 mmol) in dry DMF (4 mL) were added HATU (76 mg, 0.2 mmol) followed by (2-(2-methylpyridin-3-yl)benzofuran-5-yl)methanamine (40 mg, 0.16 mmol) and NMM (50 mg, 0.5 mmol) at 0° C. The reaction mixture was slowly warmed to rt and stirred for an additional 1.5 h. The reaction mixture was diluted with EtOAc and washed with water and brine. The organic layer was dried over anhydrous Na₂SO₄, filtered, and concentrated under reduced pressure. The resultant residue was purified by preparative TLC (30% EtOAc/petroleum ether) to afford the title compound (30 mg, 38%). LCMS-G30: m/z=481.2 [M+H]⁺; Rₜ: 11.24 min. TLC: 70% EtOAc/petroleum ether, R_{f}=0.3. ¹H NMR (400 MHz, DMSO-d₆) δ ppm 8.86-8.83 (m, 1H), 8.51 (dd, J=4.7, 1.7 Hz, 1H), 8.19 (dd, J=7.9, 1.5 Hz, 1H), 7.59 (d, J=8.2 Hz, 1H), 7.55 (m, 1H), 7.41 (dd, J=7.9, 4.9 Hz, 1H), 7.34-7.29 (m, 4H), 7.27-7.21 (m, 6H), 5.17 (s, 1H), 4.41 (dd, J=5.5, 3.7 Hz, 2H), 2.76 (s, 3H), 2.23 (s, 3H).

### Example 21

### N-((4-chloro-2-methylphenyl)(phenyl)methyl)-2-hydroxy-2-(2-(2-methylpyridin-3-yl)benzofuran-5-yl)acetamide

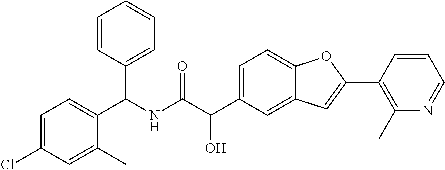 [SMILES: Cc1cc(Cl)ccc1C(NC(=O)C(O)c1ccc2oc(-c3cccnc3C)cc2c1)c1ccccc1]

### a) methyl 2-bromo-2-(2-bromobenzofuran-5-yl)acetate

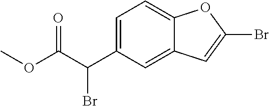 [SMILES: COC(=O)C(Br)c1ccc2oc(Br)cc2c1]

To a solution of methyl 2-(2-bromobenzofuran-5-yl)acetate (536 mg, 2 mmol) in CCl₄ (20 mL) was added NBS (427 mg, 2.4 mmol) and AIBN (394 mg, 2.4 mmol). The reaction mixture was stirred at reflux for 10 h. Water (10 mL) was added to the reaction mixture and the reaction mixture was extracted with EtOAc (3×20 mL). The combined organic layers were washed with brine, dried over Na₂SO₄, filtered, and concentrated to afford the title compound (280 mg, 40%) as a yellow oil. LCMS-P1: 347 [M+H]⁺; Rₜ: 1.673 min.

### b) 2-(2-bromobenzofuran-5-yl)-2-hydroxyacetic acid

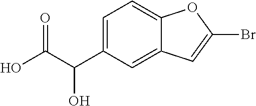 [SMILES: O=C(O)C(O)c1ccc2oc(Br)cc2c1]

The title compound was synthesized from methyl 2-bromo-2-(2-bromobenzofuran-5-yl)acetate following essentially the procedure of Example 1(f). LCMS-P1: 253/255 [M−OH]⁺; Rₜ: 1.321 min.

### c) 2-(2-bromobenzofuran-5-yl)-N-((4-chloro-2-methylphenyl)(phenyl)methyl)-2-hydroxyacetamide

The title compound was synthesized from 2-(2-bromobenzofuran-5-yl)-2-hydroxyacetic acid and (4-chloro-2-methylphenyl)(phenyl)methanamine hydrochloride following essentially the procedure of Example 2(b) (200 mg, 56%). LCMS-P1: 484/486 [M+H]⁺; Rₜ: 1.816 min.

### d) N-((4-chloro-2-methylphenyl)(phenyl)methyl)-2-hydroxy-2-(2-(2-methylpyridin-3-yl)benzofuran-5-yl)acetamide

A solution of 2-(2-bromobenzofuran-5-yl)-N-((4-chloro-2-methylphenyl)(phenyl)methyl)-2-hydroxyacetamide (200 mg, 0.41 mmol), 2-methylpyridin-3-ylboronic acid (56.2 mg, 0.41 mmol), Pd(dppf)Cl₂ (33.5 mg, 0.041 mmol), and K₂CO₃ (113 mg, 0.82 mmol) in 1,4-dioxane (10 mL) and water (2 mL) was stirred under Ar at 90° C. for 3 h. Water (10 mL) was added and the reaction mixture was extracted with EtOAc (3×10 mL). The combined organic layers were washed with brine, dried over Na₂SO₄, filtered, and concentrated. The resultant residue was purified by preparative HPLC to obtain the title compound (31 mg, 17.8%). LCMS-P1: 497 [M+H]⁺; Rₜ: 1.618 min. ¹H NMR (500 MHz, DMSO-d₆) δ ppm 8.52 (s, 1H), 8.30 (s, 1H), 7.57-7.19 (m, 7H), 7.08-6.80 (m, 7H), 6.27-6.22 (m, 1H), 5.16 (d, J=8.5 Hz, 1H), 2.78 (d, J=6.0 Hz, 3H), 2.10 (d, J=16.5 Hz, 3H).

### Example 22

### N-((4-chloro-2-methylphenyl)(phenyl)methyl)-2-(2-(2-methylpyridin-3-yl)-1-oxo-1,2,3,4-tetrahydroisoquinolin-6-yl)acetamide

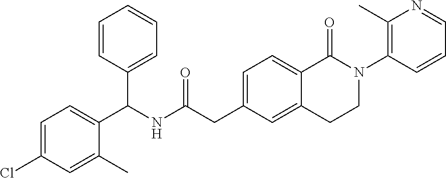 [SMILES: Cc1cc(Cl)ccc1C(NC(=O)Cc1ccc2c(c1)CCN(c1cccnc1C)C2=O)c1ccccc1]

### a) ethyl 2-(4-(3-chloropropanoyl)phenyl)acetate

A solution of ethyl phenylacetate (20 g, 0.158 mol) and 3-chloropropionyl chloride (14 mL, 0.143 mol) in CH₂Cl₂ (100 mL) was added dropwise to a suspension of aluminium chloride (48.5 g, 0.364 mol) in CH₂Cl₂ (200 mL) at 0° C. The reaction mixture was stirred at 20° C. for 24 h and then quenched with ice-water. The reaction mixture was extracted with CH₂Cl₂, dried over anhydrous Na₂SO₄, filtered, and concentrated under reduced pressure. The resultant residue was purified by column chromatography (15% EtOAc/petroleum ether) to afford the title compound (13.0 g, 32%) as a brown viscous liquid. TLC: 20% EtOAc/petroleum ether, R_{f}=0.3. ¹H NMR (400 MHz, CDCl₃) δ ppm 7.93 (d, J=8.2 Hz, 1H), 7.88-7.86 (m, 1H), 7.54-7.45 (m, 1H), 7.41 (d, J=8.2 Hz, 1H), 4.19-4.14 (m, 2H), 3.92 (t, J=6.7 Hz, 2H), 3.69 (s, 2H), 3.48-3.43 (m, 2H), 1.28-1.24 (m, 3H).

### b) 2-(1-oxo-2,3-dihydro-1H-inden-5-yl)acetic acid

Ethyl 2-(4-(3-chloropropanoyl)phenyl)acetate (13.0 g, 0.051 mol) in 95% aq. H₂SO₄ (200 mL) was heated to 100° C. for 1 h. The reaction mixture was cooled to rt and poured into ice-cold water. The reaction mixture was extracted with CH₂Cl₂, dried over anhydrous Na₂SO₄, filtered, and concentrated under reduced pressure to afford the title compound (6.0 g). TLC: 50% EtOAc/petroleum ether, R_{f}=0.2. ¹H NMR (300 MHz, CDCl₃) δ ppm 7.75-7.68 (m, 1H), 7.54-7.29 (m, 2H), 3.75 (m, 2H), 3.16-3.10 (m, 2H), 2.75-2.69 (m, 2H).

### c) methyl 2-(1-oxo-2,3-dihydro-1H-inden-5-yl)acetate

To a solution of 2-(1-oxo-2,3-dihydro-1H-inden-5-yl)acetic acid (6.0 g, 31.5 mmol) in dry MeOH (60 mL) was added a catalytic amount of H₂SO₄ (1.5 mL) and the reaction mixture was heated to 70° C. for 1 h. The reaction mixture was cooled to rt and concentrated under reduced pressure. The reaction mixture was extracted with EtOAc, dried over anhydrous Na₂SO₄, filtered, and concentrated under reduced pressure. The resultant residue was purified by column chromatography (15-20% EtOAc/petroleum ether) to afford the title compound (5.0 g, 78%) as a brown viscous liquid. TLC: 50% EtOAc/petroleum ether, R_{f}=0.4. ¹H NMR (400 MHz, CDCl₃) δ ppm 7.73-7.65 (m, 1H), 7.54-7.44 (m, 1H), 7.41-7.27 (m, 1H), 3.72 (s, 3H), 3.70 (m, 2H), 3.15-3.10 (m, 2H), 2.72-2.68 (m, 2H).

### d) methyl 2-(1-oxo-1,2,3,4-tetrahydroisoquinolin-6-yl)acetate

Methanesulfonic acid (23.5 g, 24.5 mmol) was added to a solution of methyl 2-(1-oxo-2,3-dihydro-1H-inden-5-yl)acetate (5.0 g, 24.5 mmol) in CH₂Cl₂ (50 mL) at 0° C. NaN₃ (2.38 g, 36.7 mmol) was added slowly portionwise to the reaction mixture, followed by stirring at rt for 3 h. The reaction mixture was quenched with 20% aq. NaOH and extracted with EtOAc. The organic layer was washed with water, dried over anhydrous Na₂SO₄, filtered, and concentrated under reduced pressure. The resultant residue was purified by column chromatography (2-3% MeOH/CH₂Cl₂) to afford the title compound (2.1 g, 39%) as a brown viscous liquid. TLC: 50% EtOAc/petroleum ether, R_{f}=0.2. ¹H NMR (300 MHz, MeOD-d₄) δ ppm 7.91-7.83 (m, 1H), 7.43-7.22 (m, 2H), 3.70-3.67 (m, 5H), 3.50-3.45 (m, 2H), 2.98-2.93 (m, 2H).

### e) methyl 2-(2-(2-methylpyridin-3-yl)-1-oxo-1,2,3,4-tetrahydroisoquinolin-6-yl)acetate

To a stirred solution of methyl 2-(1-oxo-1,2,3,4-tetrahydroisoquinolin-6-yl)acetate (500 mg, 2.2 mmol) in dry 1,4-dioxane (25 mL) were added 3-bromo-2-methylpyridine (390 mg, 2.2 mmol) followed by N,N′-dimethylethylenediamine (160 mg, 1.8 mmol) and K₃PO₄ (960 mg, 4.5 mmol). The solution was degassed with N₂ for 5 min. CuI (173 mg, 0.91 mmol) was added and the reaction mixture was heated to 100° C. for 24 h. The reaction mixture was filtered through Celite® and the filtrated was concentrated under reduced pressure and diluted with EtOAc. The solution was washed with water and brine, dried over anhydrous Na₂SO₄, filtered, and concentrated under reduced pressure. The resultant residue was purified by column chromatography (5% MeOH/CH₂Cl₂) to afford the title compound (260 mg, 37%) as a pale yellow liquid. TLC: 100% EtOAc, R_{f}=0.3. ¹H NMR (400 MHz, MeOD-d₄) δ ppm 8.44-8.43 (m, 1H), 8.01-7.93 (m, 1H), 7.81-7.78 (m, 1H), 7.49-7.31 (m, 3H), 4.07-4.00 (m, 1H), 3.85-3.73 (m, 3H), 3.71-3.69 (m, 3H), 3.21-3.20 (m, 2H), 2.49 (s, 3H).

### f) 2-(2-(2-methylpyridin-3-yl)-1-oxo-1,2,3,4-tetrahydroisoquinolin-6-yl)acetic acid

The title compound was synthesized from methyl 2-(2-(2-methylpyridin-3-yl)-1-oxo-1,2,3,4-tetrahydroisoquinolin-6-yl)acetate following essentially the procedure of Example 1(f). TLC: 10% MeOH/CH₂Cl₂, R_{f}=0.2.

### g) N-((4-chloro-2-methylphenyl)(phenyl)methyl)-2-(2-(2-methylpyridin-3-yl)-1-oxo-1,2,3,4-tetrahydroisoquinolin-6-yl)acetamide

The compound was synthesized from 2-(2-(2-methylpyridin-3-yl)-1-oxo-1,2,3,4-tetrahydroisoquinolin-6-yl)acetic acid and (4-chloro-2-methylphenyl)(phenyl)methanamine following essentially the procedure of Example 5 and was purified by column chromatography (5% MeOH/CH₂Cl₂) followed by preparative TLC (5% MeOH/CH₂Cl₂) to afford the title compound (40 mg, 23%). TLC: 10% MeOH/CH₂Cl₂, R_{f}=0.3. LCMS-G30: m/z=508.2 [M+H]⁺; Rₜ: 3.87 min. ¹H NMR (400 MHz, MeOD-d₄) δ ppm 8.45-8.42 (m, 1H), 8.00-7.95 (m, 1H), 7.81-7.77 (m, 1H), 7.53-7.38 (m, 2H), 7.36-7.28 (m, 5H), 7.22-7.12 (m, 5H), 6.33-6.28 (m, 1H), 4.06-3.99 (m, 1H), 3.84-3.79 (m, 1H), 3.69-3.66 (m, 2H), 3.26-3.12 (m, 2H), 2.48 (s, 3H), 2.23 (s, 3H).

### Example 23

### N-((4-chloro-2-methylphenyl)(phenyl)methyl)-2-(2-(2-methylpyridin-3-yl)-1H-benzo[d]imidazol-5-yl)acetamide

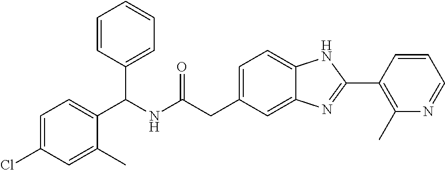 [SMILES: Cc1cc(Cl)ccc1C(NC(=O)Cc1ccc2[nH]c(-c3cccnc3C)nc2c1)c1ccccc1]

### a) methyl 2-(4-amino-3-nitrophenyl)acetate

Fuming HNO₃ (3 mL) was added dropwise to a solution of methyl 2-(4-aminophenyl)acetate (6.0 g, 36.32 mmol) in glacial AcOH (200 mL) at 0° C. The reaction mixture was allowed to warm to rt and then heated to 100° C. for 6 h. The reaction mixture was cooled to rt, concentrated under reduced pressure and azeotroped with toluene. The reaction mixture was neutralized using 10% aq. NaOH and extracted with EtOAc. The organic layer was washed with water and brine, dried over anhydrous Na₂SO₄, filtered, and concentrated under reduced pressure. The resultant residue was purified by column chromatography (20% EtOAc/petroleum ether) to afford the title compound (1.4 g, 18%) as an orange solid. TLC: 50% EtOAc/petroleum ether, R_{f}=0.5. ¹H NMR (400 MHz, DMSO-d₆) δ ppm 7.88 (d, J=1.8 Hz, 1H), 7.41 (br s, 2H), 7.31 (dd, J=8.9, 2.1 Hz, 1H), 6.97 (d, J=8.5 Hz, 1H), 3.61 (m, 5H).

### b) methyl 2-(3,4-diaminophenyl)acetate

Methyl 2-(4-amino-3-nitrophenyl)acetate (800 mg, 3.81 mmol) was hydrogenated using 10% Pd/C (120 mg) in MeOH (80 mL) under H₂ balloon pressure for 4 h. The reaction mixture was filtered through Celite® and washed through with MeOH. The solvent was concentrated to obtain the title compound (500 mg, 73%). TLC: 100% EtOAc, R_{f}=0.3. ¹H NMR (300 MHz, DMSO-d₆) δ ppm 6.41-6.37 (m, 2H), 6.24-6.21 (m, 1H), 4.43-4.35 (m, 4H), 3.55 (s, 5H).

### c) methyl 2-(2-(2-methylpyridin-3-yl)-1H-benzo[d]imidazol-5-yl)acetate

To a solution of 2-methylnicotinaldehyde (336 mg, 2.77 mmol) in EtOH (1.0 mL) was added 40% NaHSO₃ (3.3 mL) and the reaction mixture was stirred at rt for 30 min. A solution of methyl 2-(3,4-diaminophenyl)acetate (500 mg, 2.77 mmol) in EtOH (2.3 mL) was added and the reaction mixture was stirred at 90° C. for 4 h. The reaction mixture was diluted with water and extracted with EtOAc. The organic layer was washed with water and brine, dried over anhydrous Na₂SO₄, filtered, and concentrated under reduced pressure. The resultant residue was purified by column chromatography (100% EtOAc) to afford the title compound (230 mg, 29%). TLC: 100% EtOAc, R_{f}=0.2. ¹H NMR (400 MHz, DMSO-d₆) δ ppm 12.78 (br s, 1H), 8.57 (dd, J=4.8, 1.8 Hz, 1H), 8.13 (dd, J=7.8, 1.8 Hz, 1H), 7.65-7.46 (m, 2H), 7.43 (dd, J=7.8, 4.8 Hz, 1H), 7.16-7.14 (m, 1H), 3.81 (m, 2H), 3.63 (s, 3H), 2.81 (s, 3H).

### d) 2-(2-(2-methylpyridin-3-yl)-1H-benzo[d]imidazol-5-yl)acetic acid

The title compound was synthesized from methyl 2-(2-(2-methylpyridin-3-yl)-1H-benzo[d]imidazol-5-yl)acetate following essentially the procedure of Example 1(f). TLC: 20% MeOH/CH₂Cl₂, R_{f}=0.2. ¹H NMR (400 MHz, DMSO-d₆) δ ppm 8.79 (dd, J=5.2, 1.2 Hz, 1H), 8.58-8.56 (m, 1H), 7.78-7.73 (m, 3H), 7.39-7.37 (m, 1H), 3.80 (s, 2H), 2.96 (s, 3H).

### e) N-((4-chloro-2-methylphenyl)(phenyl)methyl)-2-(2-(2-methylpyridin-3-yl)-1H-benzo[d]imidazol-5-yl)acetamide

The compound was synthesized from 2-(2-(2-methylpyridin-3-yl)-1H-benzo[d]imidazol-5-yl)acetic acid and (4-chloro-2-methylphenyl)(phenyl)methanamine following essentially the procedure of Example 5 and was purified by column chromatography (100% EtOAc) followed by preparative TLC (10% MeOH/CH₂Cl₂) to afford the title compound (30 mg, 9%). TLC: 100% EtOAc, R_{f}=0.1. LCMS-T1: m/z=481.1 [M+H]⁺; Rₜ: 5.19 min. ¹H NMR (400 MHz, DMSO-d₆) δ ppm 12.87 (br s, 1H), 8.99 (d, J=8.2 Hz, 1H), 8.58 (d, J=3.4 Hz, 1H), 8.13 (d, J=7.9 Hz, 1H), 7.57-7.52 (m, 2H), 7.45-7.42 (m, 1H), 7.35-7.31 (m, 2H), 7.28-7.24 (m, 2H), 7.22-7.18 (m, 3H), 7.16-7.12 (m, 1H), 6.20 (d, J=8.2 Hz, 1H), 3.64 (s, 2H), 2.80 (s, 3H), 2.19 (s, 3H).

### Example 24

### N-((4-chloro-2-methylphenyl)(phenyl)methyl)-2-(2-(2-methylpyridin-3-yl)quinolin-6-yl)acetamide

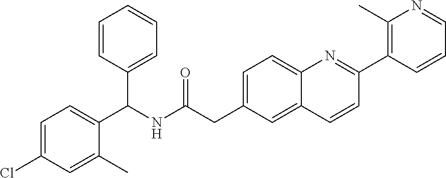 [SMILES: Cc1cc(Cl)ccc1C(NC(=O)Cc1ccc2nc(-c3cccnc3C)ccc2c1)c1ccccc1]

### a) 2-(quinolin-6-yl)acetic acid

A mixture of p-aminophenylacetic acid (20 g, 0.132 mol), FeSO₄ (4.9 g, 0.032 mol), glycerol (51 g, 0.555 mol), PhNO₂ (10.5 g, 0.086 mol), and concentrated H₂SO₄ (23 mL) was heated to 200° C. for 5 h. The reaction mixture was treated with 4 N aq. NaOH to adjust the pH to ˜10. The reaction mixture was filtered and the clear filtrate was acidified with AcOH to pH˜5. The resultant precipitate was collected by filtration to afford the title compound (12.5 g, 51%) as a brown solid. TLC: 20% MeOH/CH₂Cl₂, R_{f}=0.2. ¹H NMR (300 MHz, DMSO-d₆) δ ppm 8.85 (m, 1H), 8.31-8.28 (m, 1H), 7.96 (d, J=8.3 Hz, 1H), 7.82 (m, 1H), 7.67-7.64 (m, 1H), 7.50-7.49 (m, 1H), 3.78 (s, 2H).

### b) methyl 2-(quinolin-6-yl)acetate

2-(quinolin-6-yl)acetic acid (12.0 g, 0.064 mol) was dissolved in dry MeOH (120 mL) and cooled to 0° C. SOCl₂ (7.0 mL, 0.096 mol) was added, and the reaction mixture was refluxed for 2 h. The reaction mixture was concentrated under reduced pressure and the resultant residue was dissolved in EtOAc, washed with water and brine, dried over anhydrous Na₂SO₄, filtered, and concentrated under reduced pressure. The resultant residue was purified by column chromatography (20-25% EtOAc/petroleum ether) to afford the title compound (9.8 g, 76%). TLC: 20% MeOH/CH₂Cl₂, R_{f}=0.6. ¹H NMR (300 MHz, DMSO-d₆) δ ppm 8.86 (dd, J=4.3, 1.6 Hz, 1H), 8.32-8.29 (m, 1H), 7.97 (d, J=8.6 Hz, 1H), 7.84 (m, 1H), 7.66 (dd, J=8.8, 2.0 Hz, 1H), 7.50 (dd, J=8.2, 4.3 Hz, 1H), 3.90 (s, 2H), 3.63 (s, 3H).

### c) 6-(2-methoxy-2-oxoethyl)quinoline 1-oxide

m-CPBA (0.77 g, 4.4 mmol) was added to a solution of methyl 2-(quinolin-6-yl)acetate (0.6 g, 2.98 mmol) in DME (5 mL) at rt and stirred for 2 h. The reaction mixture was diluted with water and basified to pH ˜10 using saturated aq. K₂CO₃. The reaction mixture was extracted with EtOAc, dried over anhydrous Na₂SO₄, filtered, and concentrated under reduced pressure to afford the title compound (800 mg). TLC: 10% MeOH/CHCl₃, R_{f}=0.1. ¹H NMR (300 MHz, DMSO-d₆) δ ppm 8.55-8.53 (m, 1H), 8.48-8.45 (m, 1H), 7.95 (m, 1H), 7.86 (m, 1H), 7.72-7.69 (m, 1H), 7.45-7.41 (m, 1H), 3.92 (s, 2H), 3.63 (s, 3H).

### d) methyl 2-(2-chloroquinolin-6-yl)acetate

6-(2-methoxy-2-oxoethyl)quinoline 1-oxide (800 mg, 3.68 mmol) and POCl₃ (24 mL) was refluxed for 30 min, followed by concentration under reduced pressure. The resultant residue was treated with saturated aq. NaHCO₃ and extracted with EtOAc. The organic layer was dried over anhydrous Na₂SO₄, filtered, and concentrated under reduced pressure. The resultant residue was purified by column chromatography (20-25% EtOAc/petroleum ether) to afford the title compound (350 mg, 50%) as an off-white solid. TLC: 50% EtOAc/petroleum ether, R_{f}=0.5. ¹H NMR (400 MHz, CDCl₃) δ ppm 8.07 (d, J=8.5 Hz, 1H), 8.00 (d, J=8.8 Hz, 1H), 7.73 (s, 1H), 7.67 (dd, J=8.7, 1.9 Hz, 1H), 7.39 (d, J=8.5 Hz, 1H), 3.82 (s, 2H), 3.73 (s, 3H).

### e) methyl 2-(2-(2-methylpyridin-3-yl)quinolin-6-yl)acetate

Methyl 2-(2-chloroquinolin-6-yl)acetate (100 mg, 0.42 mmol), 2-methylpyridine-3-boronic acid (93 mg, 0.42 mmol), and Na₂CO₃ (225 mg, 2.12 mmol) were dissolved in 3:1 CH₃CN/H₂O (10 mL) and the solution was purged with Ar for 10 min. Pd(PPh₃)₄ (25 mg, 0.02 mmol) was added and the reaction mixture was heated to 100° C. for 3 h in a sealed tube. The reaction mixture was cooled to rt, diluted with water, and extracted with EtOAc. The organic layer was washed with water and brine, dried over anhydrous Na₂SO₄, filtered, and concentrated under reduced pressure to afford the title compound (110 mg). TLC: 50% EtOAc/petroleum ether, R_{f}=0.2.

### f) 2-(2-(2-methylpyridin-3-yl)quinolin-6-yl)acetic acid

The title compound was synthesized from methyl 2-(2-(2-methylpyridin-3-yl)quinolin-6-yl)acetate following essentially the procedure of Example 1(f). TLC: 10% MeOH/CHCl₃, R_{f}=0.1. ¹H NMR (400 MHz, DMSO-d₆) δ ppm 8.82-8.81 (m, 1H), 8.57 (d, J=8.5 Hz, 2H), 8.06 (d, J=8.5 Hz, 1H), 7.97 (m, 1H), 7.91-7.89 (m, 2H), 7.78 (dd, J=8.8, 2.0 Hz, 1H), 3.86 (s, 2H), 2.83 (s, 3H).

### g) N-((4-chloro-2-methylphenyl)(phenyl)methyl)-2-(2-(2-methylpyridin-3-yl)quinolin-6-yl)acetamide

The compound was synthesized from 2-(2-(2-methylpyridin-3-yl)quinolin-6-yl)acetic acid and (4-chloro-2-methylphenyl)(phenyl)methanamine following essentially the procedure of Example 5 (23 mg, 14%). TLC: 50% EtOAc/petroleum ether, R_{f}=0.3. LCMS-G7: m/z=492.2 [M+H]⁺; Rₜ: 4.06 min. ¹H NMR (400 MHz, CDCl₃) δ ppm 8.62 (dd, J=4.4, 1.2 Hz, 1H), 8.21 (d, J=8.5 Hz, 1H), 8.15 (d, J=8.5 Hz, 1H), 7.86 (d, J=7.3 Hz, 1H), 7.79 (s, 1H), 7.69 (dd, J=8.7, 1.9 Hz, 1H), 7.58 (d, J=8.2 Hz, 1H), 7.32-7.29 (m, 3H), 7.16 (s, 1H), 7.11-7.07 (m, 3H), 6.91 (d, J=8.2 Hz, 1H), 6.38 (d, J=7.6 Hz, 1H), 5.98 (d, J=7.3 Hz, 1H), 3.86 (s, 2H), 2.97-2.89 (m, 1H), 2.67 (s, 3H), 2.23 (s, 3H).

### Example 25

### 2-(2-(3,5-dimethylisoxazol-4-yl)quinolin-6-yl)-N-((2,4-dimethylphenyl)(phenyl)methyl)acetamide

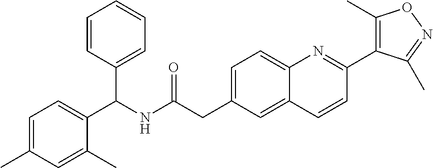 [SMILES: Cc1ccc(C(NC(=O)Cc2ccc3nc(-c4c(C)noc4C)ccc3c2)c2ccccc2)c(C)c1]

### a) methyl 2-(2-(3,5-dimethylisoxazol-4-yl)quinolin-6-yl)acetate

The title compound was synthesized from methyl 2-(2-chloroquinolin-6-yl)acetate following essentially the procedure of Example 10(c) and purified by column chromatography (50% EtOAc/petroleum ether) to obtain the title compound (45 mg, 20%) as pale yellow solid. LCMS-P1: 297.0 [M+H]⁺; Rₜ: 1.540 min.

### b) 2-(2-(3,5-dimethylisoxazol-4-yl)quinolin-6-yl)acetic acid

The title compound was synthesized from methyl 2-(2-(3,5-dimethylisoxazol-4-yl)quinolin-6-yl)acetate following essentially the procedure of Example 1(f). LCMS-P1: 283.0 [M+H]⁺; Rₜ: 1.310 min.

### c) 2-(2-(3,5-dimethylisoxazol-4-yl)quinolin-6-yl)-N-((2,4-dimethylphenyl)(phenyl)methyl)acetamide

The title compound was synthesized from 2-(2-(3,5-dimethylisoxazol-4-yl)quinolin-6-yl)acetic acid and (2,4-dimethylphenyl)(phenyl)methanamine hydrochloride following essentially the procedure of Example 2(b) and purified by preparative HPLC to obtain the title compound (26 mg, 39%) as a white solid. LCMS-P1: 476.0 [M+H]⁺; Rₜ: 1.745 min. ¹H NMR (500 MHz, DMSO-d₆) δ ppm 9.01 (d, J=8.4 Hz, 1H), 8.38 (d, J=8.4 Hz, 1H), 8.27 (d, J=1.2 Hz, 1H), 7.95 (d, J=8.4 Hz, 1H), 7.71-7.68 (m, 2H), 7.33-6.96 (m, 8H), 6.22 (d, J=8.4 Hz, 1H), 3.74 (s, 2H), 2.65 (s, 3H), 2.47 (s, 3H), 2.22 (s, 3H), 2.15 (s, 3H).

### Example 26

### N-((4-chloro-2-methylphenyl)(phenyl)methyl)-2-(2-(2-methylpyridin-3-yl)benzo[d]thiazol-6-yl)acetamide

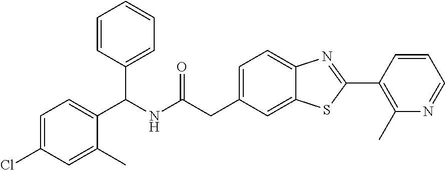 [SMILES: Cc1cc(Cl)ccc1C(NC(=O)Cc1ccc2nc(-c3cccnc3C)sc2c1)c1ccccc1]

### a) methyl 2-(4-(2-methylnicotinamido)phenyl)acetate

The compound was synthesized from methyl 2-(4-aminophenyl)acetate and 2-methylnicotinic acid following essentially the procedure of Example 5 (1.1 g, 64%). TLC: 10% MeOH/CHCl₃, R_{f}=0.3. ¹H NMR (300 MHz, DMSO-d₆) δ ppm 10.43 (s, 1H), 8.54 (dd, J=4.9, 1.2 Hz, 1H), 7.85-7.83 (m, 1H), 7.65 (d, J=8.3 Hz, 2H), 7.33 (dd, J=7.8, 4.9 Hz, 1H), 7.23 (d, J=8.3 Hz, 2H), 3.63 (s, 2H), 3.60 (s, 3H), 2.54 (s, 3H).

### b) methyl 2-(4-(2-methylpyridine-3-carbothioamido)phenyl)acetate

To a solution of methyl 2-(4-(2-methylnicotinamido)phenyl)acetate (1.1 g, 3.87 mmol) in toluene (22 mL) was added Lawesson's reagent (984 mg, 2.33 mmol). The reaction mixture was heated to reflux for 12 h, followed by concentrated under reduced pressure. The resultant residue was purified by column chromatography (3% MeOH/CHCl₃) to afford the title compound (0.45 g, 39%) as a pale yellow liquid. TLC: 10% MeOH/CHCl₃, R_{f}=0.35. ¹H NMR (300 MHz, DMSO-d₆) δ ppm 12.03 (s, 1H), 8.47 (dd, J=4.9, 1.6 Hz, 1H), 7.86 (d, J=8.3 Hz, 2H), 7.69 (dd, J=7.6, 1.4 Hz, 1H), 7.34-7.27 (m, 3H), 3.70 (s, 2H), 3.61 (s, 3H), 2.50 (s, 3H).

### c) 2-(2-(2-methylpyridin-3-yl)benzo[d]thiazol-6-yl)acetic acid

To a stirred solution of K₃Fe(CN)₆ (0.65 g, 2.0 mmol) in water (1.4 mL) at 90° C. was added a solution of methyl 2-(4-(2-methylpyridine-3-carbothioamido)phenyl)acetate (150 mg, 0.5 mmol) in EtOH (0.23 mL) and 10% aq. NaOH (1.2 mL) over a period of 2 h. The reaction mixture was stirred at 90° C. for an additional 2 h. The reaction mixture was cooled to 0° C. and the resultant precipitate was collected by filtration and dried under vacuum. The solid was treated with 3:1 CH₂Cl₂/EtOH (50 mL). The un-dissolved solid material was filtered off and the clear filtrate was concentrated under reduced pressure to afford the title compound (70 mg). TLC: 10% MeOH/CHCl₃, R_{f}=0.2.

### d) N-((4-chloro-2-methylphenyl)(phenyl)methyl)-2-(2-(2-methylpyridin-3-yl)benzo[d]thiazol-6-yl)acetamide

To a stirred solution of 2-(2-(2-methylpyridin-3-yl)benzo[d]thiazol-6-yl)acetic acid (110 mg, 0.39 mmol) and (4-chloro-2-methylphenyl)(phenyl)methanamine (90 mg, 0.39 mmol) in 1:1 CH₂Cl₂/THF (2.2 mL) at 0° C. was added DIPEA (0.14 mL, 0.78 mmol) followed by a 50% solution of ®T3P in EtOAc (0.3 mL, 0.47 mmol). The reaction mixture was stirred at rt for 2 h and then concentrated under reduced pressure. The resultant residue was diluted with EtOAc, washed with water and brine, dried over anhydrous Na₂SO₄, filtered, and concentrated under reduced pressure. The resultant residue was purified by preparative TLC (5% MeOH/CHCl₃) to afford the title compound (10 mg, 5%). TLC: 10% MeOH/CHCl₃, R_{f}=0.6. LCMS-G7: m/z=497.2 [M+H]⁺; Rₜ: 4.12 min. ¹H NMR (400 MHz, CDCl₃) δ ppm 8.65-8.64 (m, 1H), 8.18-8.16 (m, 1H), 8.11 (d, J=8.2 Hz, 1H), 7.89 (m, 1H), 7.46 (dd, J=8.4, 1.6 Hz, 1H), 7.38-7.34 (m, 1H), 7.32-7.29 (m, 2H), 7.17-7.16 (m, 1H), 7.12-7.06 (m, 3H), 6.91 (d, J=8.2 Hz, 1H), 6.36 (d, J=7.9 Hz, 1H), 5.98-5.95 (m, 1H), 3.80 (s, 2H), 2.98 (s, 3H), 2.22 (s, 3H).

### Example 27

### N-((2,4-dimethylphenyl)(phenyl)methyl)-2-(2-(2-methylpyridin-3-yl)benzo[d]thiazol-6-yl)acetamide

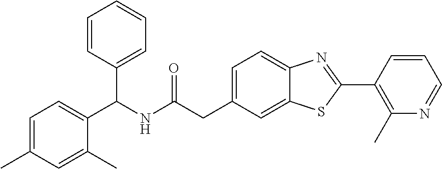 [SMILES: Cc1ccc(C(NC(=O)Cc2ccc3nc(-c4cccnc4C)sc3c2)c2ccccc2)c(C)c1]

### a) ethyl 2-(2-bromobenzo[d]thiazol-6-yl)acetate

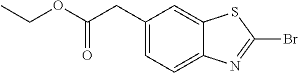 [SMILES: CCOC(=O)Cc1ccc2nc(Br)sc2c1]

To a suspension of ethyl 2-(2-aminobenzo[d]thiazol-6-yl)acetate (2.4 g, 10.2 mmol) and HBr (6 mL) in AcOH at 0° C. was slowly added a solution of NaNO₂ (774 mg, 11.22 mmol) in water (10 mL). After 30 min, the reaction mixture was allowed to warm to rt and CH₃CN (3 mL) was added followed by CuBr (3.35 g, 10.2 mmol). After 4 h, brine (20 mL) was added and the reaction mixture was extracted with EtOAc (3×30 mL). The combined organic layers were washed with brine (3×15 mL), dried over Na₂SO₄, filtered, and concentrated. The resultant residue was purified by column chromatography (20:1 petroleum ether/EtOAc) to obtain the title compound (670 mg, 22%). LC-MS: 300/302 [M+H]⁺; Rₜ: 1.69 min.

### b) ethyl 2-(2-(2-methylpyridin-3-yl)benzo[d]thiazol-6-yl)acetate

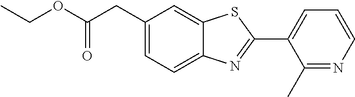 [SMILES: CCOC(=O)Cc1ccc2nc(-c3cccnc3C)sc2c1]

A solution of ethyl 2-(2-bromobenzo[d]thiazol-6-yl)acetate (240 mg, 0.8 mmol), 2-methylpyridine-3-boronic acid (164 mg, 1.2 mmol), Pd(dppf)Cl₂ (71 mg, 0.096 mmol), and K₂CO₃ (331 mg, 2.4 mmol) in 1,4-dioxane (3 mL) and water (1.5 mL) under Ar was heated under microwave irradiation at 110° C. for 30 min. Water (10 mL) was added and the reaction mixture was extracted with EtOAc (3×10 mL), washed with brine, dried over Na₂SO₄, filtered, and concentrated. The resultant residue was purified by column chromatography (33% EtOAc/petroleum ether) to obtain the title compound (168 mg, 67%). LCMS-P1: 313.0 [M+2H]⁺; Rₜ: 1.66 min.

### c) 2-(2-(2-methylpyridin-3-yl)benzo[d]thiazol-6-yl)acetic acid

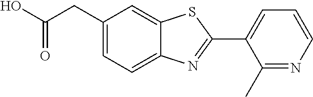 [SMILES: Cc1ncccc1-c1nc2ccc(CC(=O)O)cc2s1]

The title compound was synthesized from ethyl 2-(2-(2-methylpyridin-3-yl)benzo[d]thiazol-6-yl)acetate following essentially the procedure of Example 1(f) (51 mg, 85%). LCMS-P1: 285.1 [M+H]⁺, Rₜ: 1.04 min.

### d) N-((2,4-dimethylphenyl)(phenyl)methyl)-2-(2-(2-methylpyridin-3-yl)benzo[d]thiazol-6-yl)acetamide

The title compound was synthesized from 2-(2-(2-methylpyridin-3-yl)benzo[d]thiazol-6-yl)acetic acid and (2,4-dimethylphenyl)(phenyl)methanamine hydrochloride following essentially the procedure of Example 2(b) (19 mg, 25%). LCMS-P1: m/z 478.1 [M+H]⁺; Rₜ: 2.06 min. ¹H NMR (400 MHz, MeOD-d₄) δ ppm 8.58 (d, J=4.0 Hz, 1H), 8.22 (d, J=8.0 Hz, 1H), 8.04 (d, J=8.4 Hz, 1H), 7.99 (s, 1H), 7.54 (d, J=8.0 Hz, 1H), 7.54 (d, J=8.0 Hz, 1H), 7.47 (dd, J=7.6, 4.4 Hz, 1H), 7.33-7.17 (m, 5H), 7.00-6.94 (m, 3H), 4.88 (s, 1H), 3.78 (s, 2H), 2.86 (s, 3H), 2.28 (s, 3H), 2.19 (s, 3H).

### Example 28

### methyl ((3,5-dimethylisoxazol-4-yl)(5-(2-(((2,4-dimethylphenyl)(phenyl)methyl)amino)-2-oxoethyl)benzofuran-2-yl)methyl)carbamate

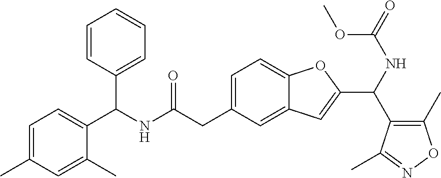

### a) 2-(2-(chloro(3,5-dimethylisoxazol-4-yl)methyl)benzofuran-5-yl)-N-((2,4-dimethylphenyl)(phenyl)methyl)acetamide

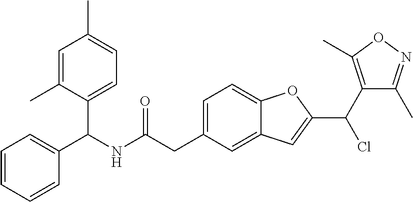 [SMILES: Cc1ccc(C(NC(=O)Cc2ccc3oc(C(Cl)c4c(C)noc4C)cc3c2)c2ccccc2)c(C)c1]

To a solution of 2-(2-((3,5-dimethylisoxazol-4-yl)(hydroxy)methyl)benzofuran-5-yl)-N-((2,4-dimethylphenyl)(phenyl)methyl)acetamide (70 mg, 0.14 mmol) in CH₂Cl₂ (10 mL) at 0° C. was added SOCl₂ (0.02 mL). The reaction mixture was stirred at rt for 2 h followed by concentration under reduced pressure to afford the title compound (70 mg, 72%).

### b) methyl ((3,5-dimethylisoxazol-4-yl)(5-(2-(((2,4-dimethylphenyl)(phenyl)methyl)amino)-2-oxoethyl)benzofuran-2-yl)methyl)carbamate

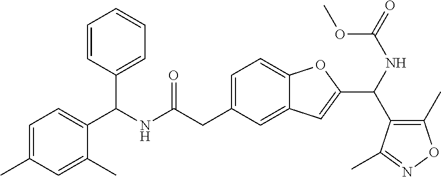

To a solution of 2-(2-(chloro(3,5-dimethylisoxazol-4-yl)methyl)benzofuran-5-yl)-N-((2,4-dimethylphenyl)(phenyl)methyl)acetamide (45 mg, 0.088 mmol) in CH₂Cl₂ (5 mL) was added methyl carbamate (13 mg, 0.176 mmol). The reaction mixture was stirred at 45° C. for 2 h. Water (30 mL) was added and the reaction mixture was extracted with EtOAc (3×30 mL). The combined organic layers were washed with brine (3×15 mL), dried over Na₂SO₄, filtered, and concentrated. The resultant residue was purified by preparative HPLC to afford the title compound (18 mg, 37%) as a white solid. LCMS-P1: 552.2 [M+H]⁺; Rₜ: 2.02 min. ¹H NMR (400 MHz, MeOD-d₄) δ ppm 7.50 (s, 1H), 7.40 (d, J=8.8 Hz, 1H), 7.23-7.28 (m, 4H), 7.14 (d, J=7.2 Hz, 2H), 6.93-6.98 (m, 3H), 6.67 (s, 1H), 6.29 (s, 1H), 6.01 (s, 1H), 3.71 (s, 3H), 3.67 (s, 2H), 2.28 (s, 6H), 2.16 (s, 3H), 2.15 (s, 3H).

### Example 29

### 2-(2-((3,5-dimethylisoxazol-4-yl)((2-methoxyethyl)amino)methyl)benzofuran-5-yl)-N-((2,4-dimethylphenyl)(phenyl)methyl)acetamide

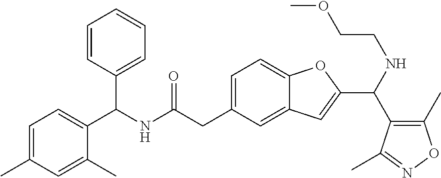

The title compound was synthesized from 2-(2-(chloro(3,5-dimethylisoxazol-4-yl)methyl)benzofuran-5-yl)-N-((2,4-dimethylphenyl)(phenyl)methyl)acetamide and 2-methoxyethanamine following essentially the procedure of Example 28(b) except carried out at rt for 12 h (15 mg, 40%). ¹H NMR (400 MHz, MeOD-d₄) δ ppm 7.50 (s, 1H), 7.37 (d, J=8.0 Hz, 1H), 7.13-7.28 (m, 6H), 6.93-6.98 (m, 3H), 6.69 (s, 1H), 6.28 (s, 1H), 5.08 (s, 1H), 3.66 (s, 2H), 3.55 (t, 2H), 3.37 (s, 3H), 2.79-2.84 (m, 2H), 2.40 (s, 3H), 2.27 (s, 3H), 2.23 (s, 3H), 2.16 (s, 3H).

### Example 30

### 2-(2-((3,5-dimethylisoxazol-4-yl)(2-hydroxy-2-methylpropoxy)methyl)benzofuran-5-yl)-N-((2,4-dimethylphenyl)(phenyl)methyl)acetamide

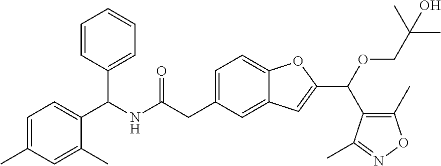 [SMILES: Cc1ccc(C(NC(=O)Cc2ccc3oc(C(OCC(C)(C)O)c4c(C)noc4C)cc3c2)c2ccccc2)c(C)c1]

To a solution of 2-(2-((3,5-dimethylisoxazol-4-yl)(hydroxy)methyl)benzofuran-5-yl)-N-((2,4-dimethylphenyl)(phenyl)methyl)acetamide (42 mg, 0.085 mmol) in xylenes (10 mL) was added 2,2-dimethyloxirane (12 mg, 0.17 mmol), KOt-Bu (19 mg, 0.17 mmol), and 18-crown-6 (4 mg, 0.017 mmol). The reaction mixture was stirred at 100° C. for 3 h. Water (30 mL) was added and the reaction mixture was extracted with EtOAc (3×30 mL). The combined organic layers were washed with brine (3×15 mL), dried over Na₂SO₄, filtered, and concentrated. The resultant residue was purified by preparative HPLC to afford the title compound (3 mg, 6%) as a white solid. LCMS-P1: 567.3 [M+H]⁺; Rₜ: 2.05 min. ¹H NMR (400 MHz, MeOD-d₄) δ ppm 7.51 (s, 1H), 7.38 (d, J=8.8 Hz, 1H), 7.24-7.29 (m, 5H), 7.14 (d, J=6.8 Hz, 2H), 6.93-6.98 (m, 3H), 6.78 (s, 1H), 6.29 (s, 1H), 5.64 (s, 1H), 3.67 (s, 1H), 3.45-3.49 (m, 2H), 2.44 (s, 3H), 2.27 (s, 3H), 2.25 (s, 3H), 2.16 (s, 3H), 1.23 (s, 6H).

### Example 31

### 2-((3,5-dimethylisoxazol-4-yl)(5-(2-(((2,4-dimethylphenyl)(phenyl)methyl)amino)-2-oxoethyl)benzofuran-2-yl)methoxy)acetic acid

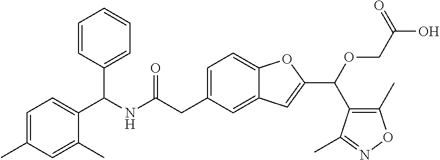 [SMILES: Cc1ccc(C(NC(=O)Cc2ccc3oc(C(OCC(=O)O)c4c(C)noc4C)cc3c2)c2ccccc2)c(C)c1]

### a) ethyl 24(3,5-dimethylisoxazol-4-yl)(5-(2-(((2,4-dimethylphenyl)(phenyl)methyl)amino)-2-oxoethyl)benzofuran-2-yl)methoxy)acetate

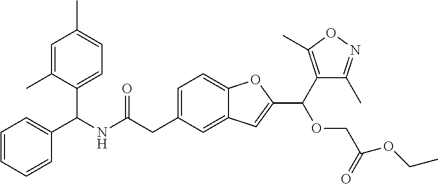 [SMILES: CCOC(=O)COC(c1cc2cc(CC(=O)NC(c3ccccc3)c3ccc(C)cc3C)ccc2o1)c1c(C)noc1C]

To a solution of 2-(2-((3,5-dimethylisoxazol-4-yl)(hydroxy)methyl)benzofuran-5-yl)-N-((2,4-dimethylphenyl)(phenyl)methyl)acetamide (100 mg, 0.2 mmol) in THF (10 mL) at rt was added NaH (9.6 mg, 60% in oil). The reaction mixture was stirred for 30 min, followed by the addition of ethyl 2-bromoacetate (33 mg, 0.2 mmol). The reaction mixture was stirred for 3 h. Water (20 mL) was added and the reaction mixture was extracted with EtOAc, washed with brine, dried over Na₂SO₄, filtered, and concentrated to afford the title compound (100 mg, 86%) as a yellowish solid. LCMS-P1: 581 [M+H]⁺; Rₜ: 1.768 min.

### b) 2-((3,5-dimethylisoxazol-4-yl)(5-(2-(((2,4-dimethylphenyl)(phenyl)methyl)amino)-2-oxoethyl)benzofuran-2-yl)methoxy)acetic acid

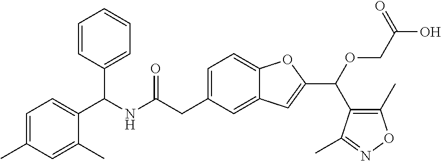 [SMILES: Cc1ccc(C(NC(=O)Cc2ccc3oc(C(OCC(=O)O)c4c(C)noc4C)cc3c2)c2ccccc2)c(C)c1]

To a solution of ethyl 24(3,5-dimethylisoxazol-4-yl)(5-(2-(((2,4-dimethylphenyl)(phenyl)methyl)amino)-2-oxoethyl)benzofuran-2-yl)methoxy)acetate (100 mg, 0.17 mmol) in THF (5 mL) and water (2 mL) was added LiOH (14.3 mg, 0.34 mmol). The reaction mixture was stirred at rt for 3 h and then acidified with 1% aq. HCl to pH=6-7. The reaction mixture was extracted with EtOAc (3×20 mL). The combined organic layers were washed with brine (3×20 mL), dried over Na₂SO₄, filtered, and concentrated. The resultant residue was purified by preparative HPLC to afford the title compound (72 mg, 77%). LCMS-P1: 553 [M+H]⁺; Rₜ: 1.743 min. ¹H NMR (400 MHz, CDCl₃) δ ppm 12.80 (br s, 1H), 8.90 (d, J=4.4 Hz, 1H), 7.50-7.46 (m, 2H), 7.32-7.16 (m, 6H), 7.00-6.89 (m, 4H), 6.18 (d, J=8.4 Hz, 1H), 5.81 (s, 1H), 4.12 (s, 2H), 3.59 (s, 2H), 2.40 (s, 3H), 2.22 (s, 3H), 2.16 (s, 3H), 2.12 (s, 3H).

### Example 32

### methyl 3-((3,5-dimethylisoxazol-4-yl)(5-(2-(((2,4-dimethylphenyl)(phenyl)methyl)amino)-2-oxoethyl)benzofuran-2-yl)methoxy)propanoate

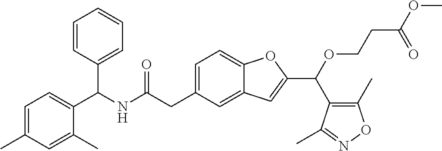

To a solution of 2-(2-((3,5-dimethylisoxazol-4-yl)(hydroxy)methyl)benzofuran-5-yl)-N-((2,4-dimethylphenyl)(phenyl)methyl)acetamide (100 mg, 0.20 mmol) in THF (20 mL) at rt under N₂ was added a catalytic amount of NaH. The reaction mixture was stirred at rt for 30 min, followed by the addition of methyl acrylate (69 mg, 0.80 mmol). The reaction mixture was stirred at rt overnight, then diluted with water (30 mL) and extracted with CH₂Cl₂ (3×30 mL). The combined organic layers were washed with 1% aq. HCl, dried over Na₂SO₄, filtered, and concentrated. The resultant residue was purified by preparative TLC (10% EtOAc/petroleum ether) to provide the title compound (23 mg, 20%) as a yellow oil. LCMS-P1: m/z 581.3 [M+1-1]⁺; Rₜ: 2.08 min. ¹H NMR (400 MHz, CDCl₃) δ ppm 7.42 (m, 2H), 7.23 (m, 3H), 7.05 (m, 3H), 6.97 (m, 1H), 6.88 (d, J=8.8 Hz, 1H), 6.75 (s, 1H), 6.61 (s, 1H), 6.38 (m, 1H), 5.92 (d, J=8.0 Hz, 1H), 3.85 (m, 2H), 3.71 (m, 5H), 2.73 (t, 2H), 2.41 (s, 3H), 2.28 (s, 3H), 2.24 (s, 3H), 2.20 (s, 3H).

### Example 33

### 3-((3,5-dimethylisoxazol-4-yl)(5-(2-(((2,4-dimethylphenyl)(phenyl)methyl)amino)-2-oxoethyl)benzofuran-2-yl)methoxy)propanoic acid

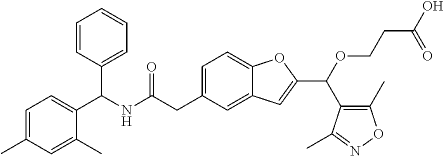 [SMILES: Cc1ccc(C(NC(=O)Cc2ccc3oc(C(OCCC(=O)O)c4c(C)noc4C)cc3c2)c2ccccc2)c(C)c1]

The title compound was synthesized from methyl 3((3,5-dimethylisoxazol-4-yl)(5-(2-(((2,4-dimethylphenyl)(phenyl)methyl)amino)-2-oxoethyl)benzofuran-2-yl)methoxy)propanoate following essentially the procedure of Example 1(f) and purified by preparative TLC (33% EtOAc/petroleum ether) to provide the title compound (12 mg, 63%) as a yellow oil. LCMS-P1: 589 [M+Na]⁺; Rₜ: 1.87 min. ¹H NMR (400 MHz, CDCl₃) δ ppm 7.42 (m, 2H), 7.23 (m, 3H), 7.05 (m, 3H), 6.97 (m, 1H), 6.88 (d, J=8.8 Hz, 1H), 6.75 (s, 1H), 6.61 (s, 1H), 6.38 (m, 1H), 5.92 (d, J=8.0 Hz 1H), 3.85 (m, 2H), 3.71 (s, 2H), 2.73 (t, 2H), 2.41 (s, 3H), 2.28 (s, 3H), 2.24 (s, 3H), 2.20 (s, 3H).

### Example 34

### 2-((3,5-dimethylisoxazol-4-yl)(5-(2-(((2,4-dimethylphenyl)(phenyl)methyl)amino)-2-oxoethyl)benzofuran-2-yl)methoxy)-N,N-dimethylacetamide

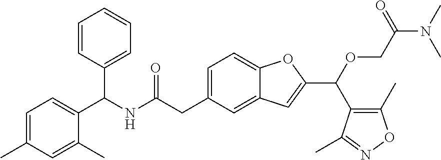

The title compound was synthesized from 2-((3,5-dimethylisoxazol-4-yl)(5-(2-(((2,4-dimethylphenyl)(phenyl)methyl)amino)-2-oxoethyl)benzofuran-2-yl)methoxy)acetic acid and dimethylamine following essentially the procedure of Example 10(b) (11 mg, 26%). ¹H NMR (400 MHz, MeOD-d₄) δ ppm 7.53 (s, 1H), 7.40 (d, J=8.0 Hz, 1H), 7.13-7.29 (m, 6H), 6.93-6.98 (m, 3H), 6.82 (s, 1H), 6.29 (s, 1H), 5.77 (s, 1H), 4.32 (s, 2H), 3.67 (s, 2H), 3.01 (s, 3H), 2.92 (s, 3H), 2.45 (s, 3H), 2.27 (s, 3H), 2.25 (s, 3H), 2.17 (s, 3H).

### Example 35

### 2-(2-((2-amino-2-methylpropoxy)(3,5-dimethylisoxazol-4-yl)methyl)benzofuran-5-yl)-N-((2,4-dimethylphenyl)(phenyl)methyl)acetamide

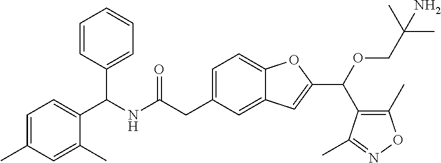 [SMILES: Cc1ccc(C(NC(=O)Cc2ccc3oc(C(OCC(C)(C)N)c4c(C)noc4C)cc3c2)c2ccccc2)c(C)c1]

To a solution of 2-(2-((3,5-dimethylisoxazol-4-yl)(hydroxy)methyl)benzofuran-5-yl)-N-((2,4-dimethylphenyl)(phenyl)methyl)acetamide (30 mg, 0.06 mmol) and 2-amino-2-methylpropan-1-ol (6 mg, 0.066 mmol) in CH₂Cl₂ (10 mL) at rt under N₂ was added TFA (0.03 mL). The reaction mixture was stirred at rt for 3 h, followed by concentration under reduced pressure. The resultant residue was purified by preparative TLC (100% EtOAc) to provide the title compound (25 mg, 73%) as a white solid. LCMS-P1: m/z 566.3 [M+H]⁺; Rₜ: 2.13 min. ¹H NMR (400 MHz, MeOD-d₄) δ ppm 7.53 (s, 1H), 7.40 (d, J=8.4 Hz, 1H), 7.14-7.28 (m, 6H), 6.94-6.98 (m, 3H), 6.81 (s, 1H), 6.29 (s, 1H), 5.67 (s, 1H), 3.67 (s, 2H), 3.45-3.49 (m, 2H), 2.45 (s, 3H), 2.26-2.27 (m, 6H), 2.17 (s, 3H), 1.26-1.28 (m, 6H).

### Example 36

### 2-((3,5-dimethylisoxazol-4-yl)(5-(2-(((2,4-dimethylphenyl)(phenyl)methyl)amino)-2-oxoethyl)benzofuran-2-yl)methoxy)-2-methylpropanoic acid

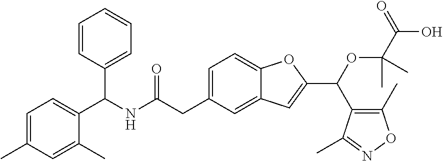 [SMILES: Cc1ccc(C(NC(=O)Cc2ccc3oc(C(OC(C)(C)C(=O)O)c4c(C)noc4C)cc3c2)c2ccccc2)c(C)c1]

### a) ethyl 24(3,5-dimethylisoxazol-4-yl)(5-(2-(((2,4-dimethylphenyl)(phenyl)methyl)amino)-2-oxoethyl)benzofuran-2-yl)methoxy)-2-methylpropanoate

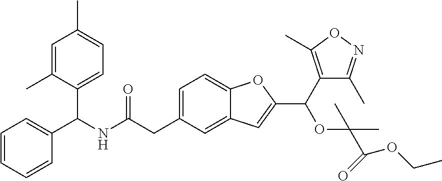 [SMILES: CCOC(=O)C(C)(C)OC(c1cc2cc(CC(=O)NC(c3ccccc3)c3ccc(C)cc3C)ccc2o1)c1c(C)noc1C]

A mixture of 2-(2-((3,5-dimethylisoxazol-4-yl)(hydroxy)methyl)benzofuran-5-yl)-N-((2,4-dimethylphenyl)(phenyl)methyl)acetamide (25 mg, 0.05 mmol), ethyl 2-bromo-2-methylpropanoate (19.5 mg, 0.1 mmol) and Cs₂CO₃ (32.5 mg, 0.1 mmol) in CH₃CN (5 mL) was stirred at reflux for 16 h. Water (20 mL) was added and the reaction mixture was extracted with EtOAc (3×20 mL), washed with brine, and dried over Na₂SO₄, filtered, and concentrated to afford the title compound (12 mg, 40%). LCMS-P1: 609.3 [M+H]⁺; Rₜ: 1.950 min.

### b) 2-((3,5-dimethylisoxazol-4-yl)(5-(2-(((2,4-dimethylphenyl)(phenyl)methyl)amino)-2-oxoethyl)benzofuran-2-yl)methoxy)-2-methylpropanoic acid

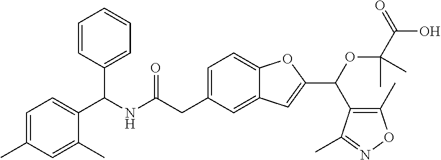 [SMILES: Cc1ccc(C(NC(=O)Cc2ccc3oc(C(OC(C)(C)C(=O)O)c4c(C)noc4C)cc3c2)c2ccccc2)c(C)c1]

To a solution of ethyl 24(3,5-dimethylisoxazol-4-yl)(5-(2-((2,4-dimethylphenyl)(phenyl)methyl)amino)-2-oxoethyl)benzofuran-2-yl)methoxy)-2-methylpropanoate (12 mg, 0.02 mmol) in THF (5 mL) and water (2 mL) was added LiOH (1.7 mg, 0.04 mmol). The reaction mixture was stirred at rt for 3 h and then acidified with 1% aq. HCl to pH=6˜7. The reaction mixture was extracted with EtOAc (3×20 mL). The combined organic layers were washed with brine (3×20 mL), dried over Na₂SO₄, filtered, and concentrated. The resultant residue was purified by preparative HPLC to obtain the title compound (2 mg, 17%). LCMS-P1: 581.3 [M+H]⁺; Rₜ: 1.779 min. ¹H NMR (400 MHz, CDCl₃) δ ppm 7.32-7.30 (m, 2H), 7.19-7.06 (m, 4H), 6.97-6.80 (m, 4H), 6.65 (d, J=7.6 Hz, 1H), 6.46 (s, 1H), 6.29 (d, J=8.0 Hz, 1H), 5.90 (d, J=8.0 Hz, 1H), 5.64 (s, 1H), 3.59 (s, 2H), 2.38 (s, 3H), 2.20 (s, 6H), 2.12 (s, 3H), 1.47 (s, 3H), 1.45 (s, 3H).

### Example 37

### 1-((3,5-dimethylisoxazol-4-yl)(5-(2-(((2,4-dimethylphenyl)(phenyl)methyl)amino)-2-oxoethyl)benzofuran-2-yl)methyl)azetidine-3-carboxylic acid

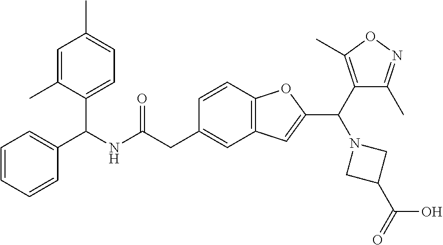 [SMILES: Cc1ccc(C(NC(=O)Cc2ccc3oc(C(c4c(C)noc4C)N4CC(C(=O)O)C4)cc3c2)c2ccccc2)c(C)c1]

### a) methyl 1-((3,5-dimethylisoxazol-4-yl)(5-(2-(((2,4-dimethylphenyl)(phenyl)methyl)amino)-2-oxoethyl)benzofuran-2-yl)methyl)azetidine-3-carboxylate

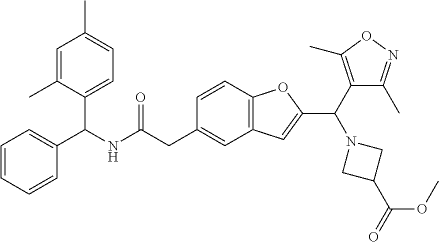 [SMILES: COC(=O)C1CN(C(c2cc3cc(CC(=O)NC(c4ccccc4)c4ccc(C)cc4C)ccc3o2)c2c(C)noc2C)C1]

2-(2-(Chloro(3,5-dimethylisoxazol-4-yl)methyl)benzofuran-5-yl)-N-((2,4-dimethylphenyl)(phenyl)methyl)acetamide (55 mg, 0.11 mmol) was added to a solution of methyl azetidine-3-carboxylate hydrochloride (30 mg, 0.20 mmol) and K₂CO₃ (30 mg, 0.22 mmol) in CH₃CN (10 mL). The mixture was stirred at rt for 12 h. Water (30 mL) was then added and the mixture was extracted with ethyl acetate (30 mL×3). The combined extracts were washed with brine (15 mL×3) and dried over Na₂SO₄. The solvent was concentrated under reduced pressure to to give title compound (48 mg, yield 73.8%). LCMS-A012: no mass; Rt=1.78 min.

### b) 1-((3,5-dimethylisoxazol-4-yl)(5-(2-(((2,4-dimethylphenyl)(phenyl)methyl)amino)-2-oxoethyl)benzofuran-2-yl)methyl)azetidine-3-carboxylic acid

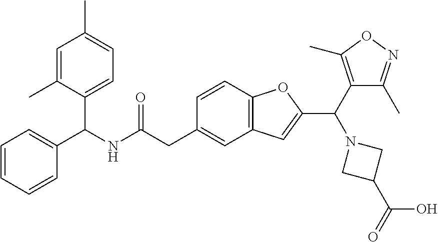 [SMILES: Cc1ccc(C(NC(=O)Cc2ccc3oc(C(c4c(C)noc4C)N4CC(C(=O)O)C4)cc3c2)c2ccccc2)c(C)c1]

The title compound was synthesized from methyl 1-((3,5-dimethylisoxazol-4-yl)(5-(2-(((2,4-dimethylphenyl)(phenyl)methyl)amino)-2-oxoethyl)benzofuran-2-yl)methyl)azetidine-3-carboxylate following essentially the procedure of Example 31 (b) (15 mg, Yield: 32.6%). LCMS-A024: 600 [M+Na]⁺. Rt=1.47 min. ¹H NMR (MeOH-d₄, 400 MHz): δ 7.50 (s, 1H), 7.37 (d, J=8.4 Hz, 1H), 7.28-7.23 (m, 4H), 7.14 (d, J=7.2 Hz, 2H), 6.98 (s, 1H), 6.93 (m, 2H), 6.75 (s, 1H), 6.28 (s, 1H), 5.00 (s, 1H), 3.99-3.95 (m, 1H), 3.87-3.79 (m, 2H), 3.77-3.71 (m, 1H), 3.67 (s, 2H), 3.56-3.47 (m, 1H), 2.40 (s, 3H), 2.27 (s, 3H), 2.26 (s, 3H), 2.17 (s, 3H).

### Example 38

### 2-(2-((3-aminoazetidin-1-yl)(3,5-dimethylisoxazol-4-yl)methyl)benzofuran-5-yl)-N-((2,4-dimethylphenyl)(phenyl)methyl)acetamide

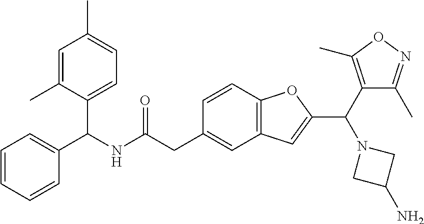 [SMILES: Cc1ccc(C(NC(=O)Cc2ccc3oc(C(c4c(C)noc4C)N4CC(N)C4)cc3c2)c2ccccc2)c(C)c1]

### a) tert-butyl (1-((3,5-dimethylisoxazol-4-yl)(5-(2-(((2,4-dimethylphenyl)(phenyl)methyl)amino)-2-oxoethyl)benzofuran-2-yl)methyl)azetidin-3-yl)carbamate

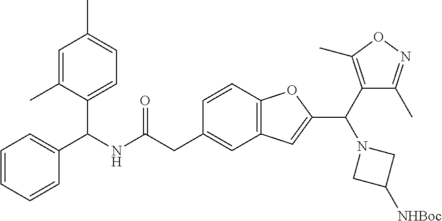 [SMILES: Cc1ccc(C(NC(=O)Cc2ccc3oc(C(c4c(C)noc4C)N4CC(NC(=O)OC(C)(C)C)C4)cc3c2)c2ccccc2)c(C)c1]

The title compound was synthesized from 2-(2-(chloro(3,5-dimethylisoxazol-4-yl)methyl)benzofuran-5-yl)-N-((2,4-dimethylphenyl)(phenyl)methyl)acetamide following essentially the procedure of Example 37 (a) (52 mg, yield 73%). LCMS-A010: 649.3[M+H]⁺; Rt=2.08 min.

### b) 2-(2-((3-aminoazetidin-1-yl)(3,5-dimethylisoxazol-4-yl)methyl)benzofuran-5-yl)-N-((2,4-dimethylphenyl)(phenyl)methyl)acetamide

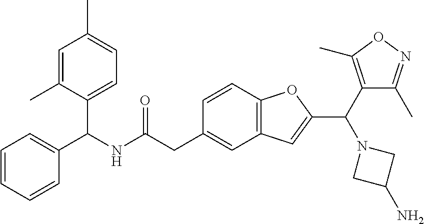 [SMILES: Cc1ccc(C(NC(=O)Cc2ccc3oc(C(c4c(C)noc4C)N4CC(N)C4)cc3c2)c2ccccc2)c(C)c1]

HCl solution in MeOH (3 mL) was added to a suspension of tert-butyl (1-((3,5-dimethylisoxazol-4-yl)(5-(2-(((2,4-dimethylphenyl)(phenyl)methyl)amino)-2-oxoethyl)benzofuran-2-yl)methyl)azetidin-3-yl)carbamate (38 mg, 0.059 mmol) in MeOH (0.5 mL). After stirring at rt for 1 h, the mixture was evaporated under reduced pressure to give a yellow oil, which was purified by Pre-TLC on silica gel (EtOAc) to provide title compound (3 mg, yield: 9%). ¹H NMR (MeOH-d₄, 400 MHz): δ 7.48 (s, 1H), 7.34 (d, J=8.4 Hz, 1H), 7.28-7.20 (m, 4H), 7.14 (d, J=6.8 Hz, 2H), 6.98 (s, 1H), 6.93-6.92 (m, 2H), 6.64 (s, 1H), 6.28 (s, 1H), 4.61 (s, 1H), 3.72-3.70 (m, 1H), 3.65 (s, 2H), 3.53-3.54 (m, 1H), 3.50-3.48 (m, 1H), 2.93-2.92 (m, 1H), 2.84-2.83 (m, 1H), 2.53 (s, 3H), 2.28 (s, 3H), 2.27 (s, 3H), 2.16 (s, 3H).

### Example 39

### N-((2,4-dimethylphenyl)(phenyl)methyl)-2-(2-(piperazin-1-yl(pyridin-4-yl)methyl)benzofuran-5-yl)acetamide

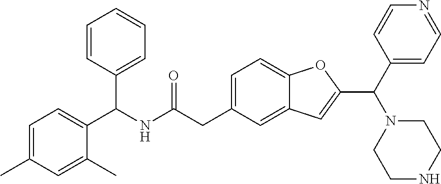 [SMILES: Cc1ccc(C(NC(=O)Cc2ccc3oc(C(c4ccncc4)N4CCNCC4)cc3c2)c2ccccc2)c(C)c1]

### a) methyl 2-(2-(hydroxy(pyridin-4-yl)methyl)benzofuran-5-yl)acetate

The title compound was synthesized from methyl 2-(2-bromobenzofuran-5-yl)acetate following essentially the procedure of Example 13 (a) (110 mg, yield 12.3%). LCMS-A024: 298.1 [M+H]⁺; Rt=1.14 min.

### b) 2-(2-(hydroxy(pyridin-4-yl)methyl)benzofuran-5-yl)acetic acid

The title compound was synthesized from methyl 2-(2-(hydroxy(pyridin-4-yl)methyl)benzofuran-5-yl)acetate following essentially the procedure of Example 1 (f) (57 mg, yield 100%). LCMS-A024: 284.1 [M+H]⁺; Rt=1.18 min.

### c) N-((2,4-dimethylphenyl)(phenyl)methyl)-2-(2-(hydroxy(pyridin-4-yl)methyl)benzofuran-5-yl)acetamide

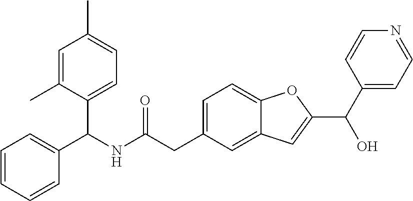 [SMILES: Cc1ccc(C(NC(=O)Cc2ccc3oc(C(O)c4ccncc4)cc3c2)c2ccccc2)c(C)c1]

The title compound was synthesized from 2-(2-(hydroxy(pyridin-4-yl)methyl)benzofuran-5-yl)acetic acid and (2,4-dimethylphenyl)(phenyl)methanamine following essentially the procedure of Example 12 (e) (11 mg, yield: 11.6%). LCMS-A024: 477.2 [M+H]⁺; Rt=1.45 min. ¹H NMR (CDCl₃, 400 MHz): δ 8.65 (s, 2H), 7.45-7.40 (m, 4H), 7.24-7.18 (m, 4H), 7.05-7.03 (m, 2H), 6.97 (s, 1H), 6.88 (d, J=8.4 Hz, 1H), 6.73 (d, J=8.0 Hz, 1H), 6.55 (s, 1H), 6.35 (d, J=8.4 Hz, 1H), 5.96 (s, 1H), 5.9 (d, J=8.0 Hz, 1H), 3.70 (s, 2H), 2.28 (s, 3H), 2.20 (s, 3H).

### d) 2-(2-(chloro(pyridin-4-yl)methyl)benzofuran-5-yl)-N-((2,4-dimethylphenyl)(phenyl)methyl)acetamide

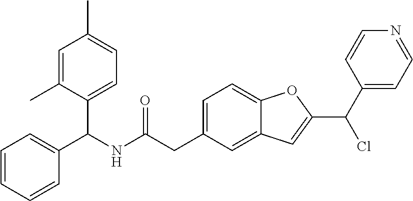 [SMILES: Cc1ccc(C(NC(=O)Cc2ccc3oc(C(Cl)c4ccncc4)cc3c2)c2ccccc2)c(C)c1]

The title compound was synthesized from N-((2,4-dimethylphenyl)(phenyl)methyl)-2-(2-(hydroxy(pyridin-4-yl)methyl)benzofuran-5-yl)acetamide following essentially the procedure of Example 28 (a).

### e) tert-butyl 4-((5-(2-(((2,4-dimethylphenyl)(phenyl)methyl)amino)-2-oxoethyl)benzofuran-2-yl)(pyridin-4-yl)methyl)piperazine-1-carboxylate

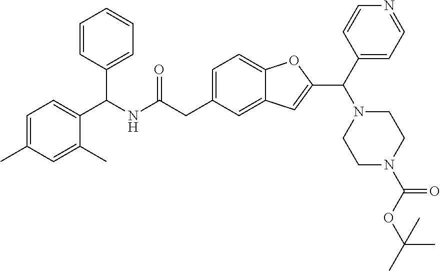 [SMILES: Cc1ccc(C(NC(=O)Cc2ccc3oc(C(c4ccncc4)N4CCN(C(=O)OC(C)(C)C)CC4)cc3c2)c2ccccc2)c(C)c1]

tert-Butyl piperazine-1-carboxylate (38 mg, 0.2 mmol) was added to the mixture of 2-(2-(chloro(pyridin-4-yl)methyl)benzofuran-5-yl)-N-((2,4-dimethylphenyl)(phenyl)methyl)acetamide (50 mg, 0.1 mmol) DIPEA (26 mg, 0.2 mmol) in CH₃CN (4 mL). The reaction was stirred at 82° C. for 20 h. Then water (5 mL) was added. The mixture was extracted with EtOAc (5 mL×3), dried over Na₂SO₄, filtered, and concentrated under reduced pressure to give tert-butyl 4-((5-(2-(((2,4-dimethylphenyl)(phenyl)methyl)amino)-2-oxoethyl)benzofuran-2-yl)(pyridin-4-yl)methyl)piperazine-1-carboxylate as a crude product. LCMS-A012: 645.3 [M+H]⁺; Rt=1.63 min.

### f) N-((2,4-dimethylphenyl)(phenyl)methyl)-2-(2-(piperazin-1-yl(pyridin-4-yl)methyl)benzofuran-5-yl)acetamide

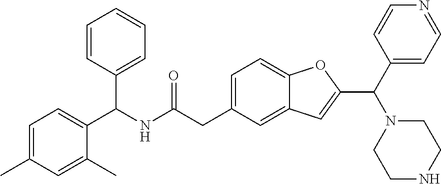 [SMILES: Cc1ccc(C(NC(=O)Cc2ccc3oc(C(c4ccncc4)N4CCNCC4)cc3c2)c2ccccc2)c(C)c1]

A solution of tert-butyl 4-((5-(2-(((2,4-dimethylphenyl)(phenyl)methyl)amino)-2-oxoethyl)benzofuran-2-yl)(pyridin-4-yl)methyl)piperazine-1-carboxylate (70 mg, 11 mmol) in TFA/CH₂Cl₂ (1.5 mL/5 mL) was stirred at rt for 1 h. The reaction mixture was then was neutralized with diluted NaOH to pH ˜5-6 and extracted with EtOAc (5 mL×3). The organic layers were dried over Na₂SO₄, filtered, and concentrated under reduced pressure. The crude product was purified by reverse phase HPLC using water/acetonitrile with 0.05% TFA to give 5 mg of N-((2,4-dimethylphenyl)(phenyl)methyl)-2-(2-(piperazin-1-yl(pyridin-4-yl)methyl)benzofuran-5-yl)acetamide. LCMS-A012: 545.3 [M+H]⁺; Rt=1.49 min. ¹H NMR (CDCl₃, 400 MHz): δ 8.53 (d, J=6 Hz, 2H), 7.62 (d, J=6 Hz, 2H), 7.51 (s, 1H), 7.38 (d, J=8.4 Hz, 1H), 7.30-7.23 (m, 4H), 7.16 (d, J=6.8 Hz, 2H), 6.70-6.91 (m, 3H), 6.83 (s, 1H), 6.28 (s, 1H), 4.64 (s, 1H), 3.66 (s, 2H), 3.02-3.00 (m, 4H), 2.58 (s, 4H), 2.27 (s, 3H), 2.17 (s, 3H).

### Example 40

### N-(1-(3,5-dimethylpyridin-2-yl)-4-methylpentyl)-2-(2-(2-methylpyridin-3-yl)quinolin-6-yl)acetamide

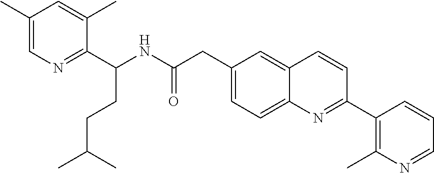 [SMILES: Cc1cnc(C(CCC(C)C)NC(=O)Cc2ccc3nc(-c4cccnc4C)ccc3c2)c(C)c1]

### a) 2-(2-chloroquinolin-6-yl)acetic acid

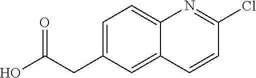 [SMILES: O=C(O)Cc1ccc2nc(Cl)ccc2c1]

This compound was synthesized from 6-(2-methoxy-2-oxoethyl)quinoline 1-oxide essentially as described in example 24 (d) (177 mg, yield 28.6%). LCMS-A024: 222.70.0 [M+H]⁺; Rt: 1.406 min.

### b) 2-(2-chloroquinolin-6-yl)-N-(1-(3,5-dimethylpyridin-2-yl)-4-methylpentyl)acetamide

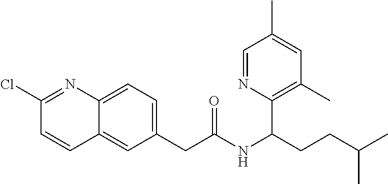 [SMILES: Cc1cnc(C(CCC(C)C)NC(=O)Cc2ccc3nc(Cl)ccc3c2)c(C)c1]

This compound was synthesized from 2-(2-chloroquinolin-6-yl)acetic acid and 1-(3,5-dimethylpyridin-2-yl)-4-methylpentan-1-amine essentially as described in example 2 (b) (60 mg, yield: 32.4%). LCMS-A010: 410.30 [M+H]⁺; Rt: 2.173 min.

### c) N-(1-(3,5-dimethylpyridin-2-yl)-4-methylpentyl)-2-(2-(2-methylpyridin-3-yl)quinolin-6-yl)acetamide

This compound was synthesized from (2-methylpyridin-3-yl)boronic acid and 2-(2-chloroquinolin-6-yl)-N-(1-(3,5-dimethylpyridin-2-yl)-4-methylpentyl)acetamide essentially as described in example 11 (c) (20 mg, yield: 29.4%). LCMS-A012: 467.30 [M+H]⁺; Rt=1.973 min. ¹H NMR (DMSO-d₆, 400 MHz): δ 8.59 (d, J=6 Hz, 1H), 8.21 (d, J=8 Hz 1H), 8.10 (d, J=8.4 Hz, 2H), 7.84 (d, J=9.6 Hz, 1H), 7.80 (s, 1H), 7.68 (d, J=8.4 Hz, 1H), 7.54 (d, J=8.8 Hz, 1H), 7.30-7.26 (m, 2H), 6.99 (d, J=8 Hz, 1H), 5.30-5.25 (m, 1H), 3.78 (s, 2H), 2.65 (s, 3H), 2.34 (s, 3H), 2.27 (s, 3H), 1.79-1.58 (m, 2H), 1.47-1.41 (m, 1H), 1.11-1.05 (m, 2H), 0.78-0.76 (m, 6H).

### Example 41

### 2-(2-(3,5-dimethylisoxazol-4-yl)benzofuran-5-yl)-N-(1-(2,4-dimethylphenyl)-2-isopropoxyethyl)acetamide

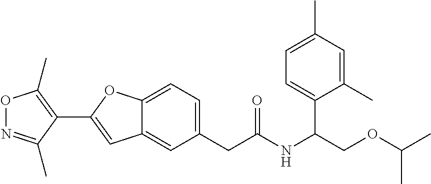 [SMILES: Cc1ccc(C(COC(C)C)NC(=O)Cc2ccc3oc(-c4c(C)noc4C)cc3c2)c(C)c1]

### (a) 2-(chloromethoxy)propane

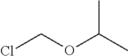

Dry hydrogen chloride was bubbled into a solution of (CH₂O)ₙ (11 g, 0.34 mol) in propan-2-ol (20 g, 0.34 mol) at 15-20° C. until all the solid was dissolved. The organic phase was separated and dried with CaCl₂. The title compound (15.6 g, yield: 49.6%) was purified by distillation. ¹H NMR (CDCl₃, 500 MHz): δ 5.54 (s, 2H), 4.10-4.00 (m, 1H), 1.23 (s, 3H), 1.22 (S, 3H).

### (b) 2-isopropoxyacetonitrile

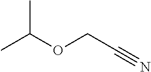

A well-stirred mixture of CuCN (2.31 g, 0.26 mol) in diethyl ether (30 mL) was heated to reflux and 2-(chloromethoxy)propane (10.8 g, 0.1 mol) in diethyl ether (30 mL) was added slowly over 1 h. The solid was removed by filtration. Title compound was obtained by distillation (3.3 g, yield: 34%). ¹H NMR (CDCl₃, 500 MHz): δ 4.24 (s, 2H), 3.86-3.77 (m, 1H), 1.23 (s, 3H), 1.21 (S, 3H).

### (c) 1-(2,4-dimethylphenyl)-2-isopropoxyethanamine

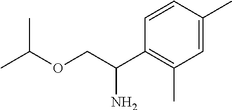

To a solution of 2-isopropoxyacetonitrile (500 mg, 0.005 mol) in THF (100 mL) was added (2,4-dimethylphenyl)magnesium bromide (5.225 g, 0.025 mol) at 0° C. under nitrogen. To the mixture was added MeOH (5 mL) followed by addition of NaBH₄ (0.028 g, 0.75 mol) at 0° C. To the mixture was added water (5 mL) and the mixture was extracted with EtOAc (10 mL×3). The organic layers were dried over Na₂SO₄, filtered, and concentrated under reduced pressure. Title compound was obtained by flash column (269 mg, yield: 26%). LCMS-A024: 208.7 [M+H]⁺; Rt=1.30 min.

### d) 2-(2-(3,5-dimethylisoxazol-4-yl)benzofuran-5-yl)-N-(1-(2,4-dimethylphenyl)-2-isopropoxyethyl)acetamide

This compound was synthesized from 2-(2-(3,5-dimethylisoxazol-4-yl)benzofuran-5-yl)acetic acid and 1-(2,4-dimethylphenyl)-2-isopropoxyethanamine essentially as described in Example 2 (b) to give title compound (56 mg, yield: 30.4%). LCMS-A024: 461.7 [M+14]⁺; Rₜ=1.47 min. ¹H NMR (DMSO-d₆, 400 MHz): δ 8.46 (d, J=8.0 Hz, 1H), 7.51-7.49 (m, 2H), 7.20 (d, J=8.0 Hz, 2H), 7.03 (s, 1H), 6.96-6.93 (m, 2H), 5.10-5.04 (m, 1H), 3.56-3.50 (m, 3H), 3.45-3.47 (d, J=6.8 Hz, 2H), 2.65 (s, 3H), 2.43 (s, 3H), 2.23 (s, 3H), 2.20 (s, 3H), 1.04-1.00 (m, 6H)

### Example 42

### N-((2,4-dimethylphenyl)(phenyl)methyl)-2-(3-(2-methylpyridin-3-yl)quinolin-6-yl)acetamide

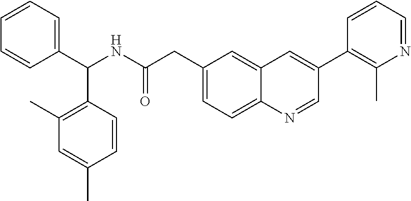 [SMILES: Cc1ccc(C(NC(=O)Cc2ccc3ncc(-c4cccnc4C)cc3c2)c2ccccc2)c(C)c1]

### a) methyl 2-(3-bromoquinolin-6-yl)acetate

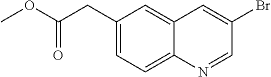 [SMILES: COC(=O)Cc1ccc2ncc(Br)cc2c1]

Bromine (316 mg, 2 mmol) was added to a solution of methyl 2-(quinolin-6-yl)acetate (179 mg, 1 mmol) in CCl₄ (15 mL). Then the reaction mixture was heated to 100° C. for 2 h. Pyridine (158 mg, 2 mmol) was added to the mixture. Then the reaction mixture was reflux for an additional 2 h. After cooling to rt, CH₂Cl₂ (15 mL) and NaHCO₃ (15 mL) were added to the mixture. The organic layer was separated, washed with brine and water, dried over Na₂SO₄, and concentrated under reduced pressure. The resulting residue was purified by reverse phase HPLC using water/acetonitrile with 0.05% TFA to get title compound (80 mg, gelid: 29%). LCMS-A012: 280.99 [M+H]⁺; Rt: 1.909 min.

### b) 2-(3-bromoquinolin-6-yl)acetic acid

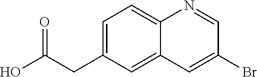 [SMILES: O=C(O)Cc1ccc2ncc(Br)cc2c1]

This compound was synthesized from methyl 2-(3-bromoquinolin-6-yl)acetate essentially as described in Example 31 (b) (130 mg, yield 96.3%). LCMS-A024: 265.97 [M+H]⁺; Rt=1.39 min.

### c) 2-(3-bromoquinolin-6-yl)-N-((2,4-dimethylphenyl)(phenyl)methyl)acetamide

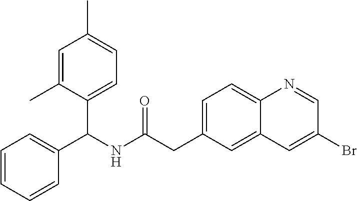 [SMILES: Cc1ccc(C(NC(=O)Cc2ccc3ncc(Br)cc3c2)c2ccccc2)c(C)c1]

This compound was prepared from 2-(3-bromoquinolin-6-yl)acetic acid and (2,4-dimethylphenyl)(phenyl)methanamine as described in Example 2 (b) (150 mg, yield: 65.5%). LCMS-A010: 459.10 [M+H]⁺; Rt: 2.24 min.

### d) N-((2,4-dimethylphenyl)(phenyl)methyl)-2-(3-(2-methylpyridin-3-yl)quinolin-6-yl)acetamide

This compound was prepared from (2-methylpyridin-3-yl)boronic acid and 2-(3-bromoquinolin-6-yl)-N-((2,4-dimethylphenyl)(phenyl)methyl)acetamide as described in Example 11 (c) (16 mg, yield: 31%). LCMS-A012: 472.23 [M+H]⁺; Rt=1.998 min. ¹H NMR (DMSO-d₆, 400 MHz): δ 9.02 (d, J=8 Hz, 1H), 8.89 (d, J=2.4 Hz, 1H), 8.54 (d, J=5.2 Hz, 1H), 8.36 (s, 1H), 8.01 (d, J=8.8 Hz, 1H), 7.85 (s, 1H), 7.79 (d, J=7.6 Hz, 1H), 7.74 (d, J=8.4 Hz, 1H), 7.40-7.37 (m, 1H), 7.33-7.29 (m, 2H), 7.25-718 (m, 3H), 7.02-6.94 (m, 3H), 6.21 (d, J=8.4 Hz, 1H), 3.76 (s, 2H), 2.49 (s, 3H), 2.22 (s, 3H), 2.16 (s, 3H).

### Example 43

### 2-(2-((2-amino-2-oxoethoxy)(pyridin-4-yl)methyl)benzofuran-5-yl)-N-((2,4-dimethylphenyl)(phenyl)methyl)acetamide

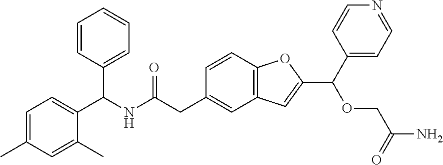 [SMILES: Cc1ccc(C(NC(=O)Cc2ccc3oc(C(OCC(N)=O)c4ccncc4)cc3c2)c2ccccc2)c(C)c1]

NH₄Cl (4 mg, 0.066 mmol), HOBt (16 mg, 0.12 mmol), EDCI (15 mg, 0.12 mmol), and DIPEA (35 mg, 0.18 mmol) were added to a solution of compound 2-((5-(2-(((2,4-dimethylphenyl)(phenyl)methyl)amino)-2-oxoethyl)benzofuran-2-yl)(pyridin-4-yl)methoxy)acetic acid (32 mg, 0.06 mmol) in CH₂Cl₂ (8 mL). The mixture was stirred at rt for 8 h. Water (50 mL) was then added to reaction mixture, and the mixture was extracted with ethyl acetate (30 mL×3). The combined extracts were dried over Na₂SO₄ and concentrated under reduced pressure to yield a red oil. The oil was purified by silica gel chromatography using 100% EtOAc to give 2-(2-((2-amino-2-oxoethoxy)(pyridin-4-yl)methyl)benzofuran-5-yl)-N-((2,4-dimethylphenyl)(phenyl)methyl)acetamide as a white solid (6 mg, yield 19%). LCMS-A024: 534.20[M+H]⁺; Rt=1.20 min. ¹H NMR (MeOH-d₄, 400 MHz): δ 8.58 (d, J=5.6 Hz, 2H), 7.63 (d, J=6.0 Hz, 2H), 7.53 (s, 1H), 7.38 (d, J=8.4 Hz, 1H), 7.30-7.23 (m, 4H), 7.14 (d, J=6.8 Hz, 2H), 6.98 (s, 1H), 6.94-6.92 (m, 2H), 6.87 (s, 1H), 6.28 (s, 1H), 5.86 (s, 1H), 4.11 (d, J=2.4 Hz, 2H), 3.66 (s, 2H), 2.27 (s, 3H), 2.16 (s, 3H).

### Example 44

### 2-(2-((2-amino ethoxy)(3,5-dimethylisoxazol-4-yl)methyl)benzofuran-5-yl)-N-((2,4-dimethylphenyl)(phenyl)methyl)acetamide

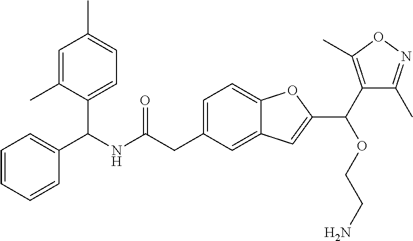 [SMILES: Cc1ccc(C(NC(=O)Cc2ccc3oc(C(OCCN)c4c(C)noc4C)cc3c2)c2ccccc2)c(C)c1]

### a) 2-(2-((3,5-dimethylisoxazol-4-yl)(2-(1,3-dioxoisoindolin-2-yl)ethoxy)methyl)benzofuran-5-yl)-N-((2,4-dimethylphenyl)(phenyl)methyl)acetamide

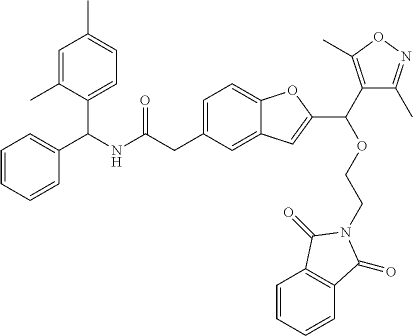 [SMILES: Cc1ccc(C(NC(=O)Cc2ccc3oc(C(OCCN4C(=O)c5ccccc5C4=O)c4c(C)noc4C)cc3c2)c2ccccc2)c(C)c1]

A solution of 2-(2-(chloro(3,5-dimethylisoxazol-4-yl)methyl)benzofuran-5-yl)-N-((2,4-dimethylphenyl)(phenyl)methyl)acetamide (61 mg, 0.12 mmol) and 2-(2-hydroxyethyl)isoindoline-1,3-dione (69 mg, 0.36 mmol) in CH₂Cl₂ (8 mL) was stirred at rt for 24 h. Then the solvent was concentrated under reduced pressure, and the resulting residue was purified with silica gel column chromatography (Petroleum ether/EtOAc=2/1) to obtain title compound (47 mg, yield: 59%) as a white solid.

### b) 2-(2-((2-aminoethoxy)(3,5-dimethylisoxazol-4-yl)methyl)benzofuran-5-yl)-N-((2,4-dimethylphenyl)(phenyl)methyl)acetamide

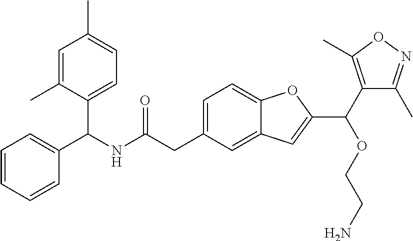 [SMILES: Cc1ccc(C(NC(=O)Cc2ccc3oc(C(OCCN)c4c(C)noc4C)cc3c2)c2ccccc2)c(C)c1]

Hydrazine hydrate (0.1 mL, 2.16 mmol) was added to a solution of 2-(2-((3,5-dimethylisoxazol-4-yl)(2-(1,3-dioxoisoindolin-2-yl)ethoxy)methyl)benzofuran-5-yl)-N-((2,4-dimethylphenyl)(phenyl)methyl)acetamide (71 mg, 0.108 mmol) in MeOH (6 mL). The mixture was stirred at 40° C. for 1 h. The solvent was concentrated under reduced pressure and the resulting residue was purified by reverse phase HPLC using water/acetonitrile with 0.05% TFA to obtain title compound (31 mg, yield: 54%) as a white solid. LCMS-A024: 538.2[M+H]⁺; Rt=1.453 min. ¹H NMR (DMSO-d₆, 400 MHz): δ 7.50-7.45 (m, 2H), 7.32-7.29 (m, 2H), 7.26-7.20 (m, 2H), 7.16 (d, J=8.8 Hz, 2H), 6.99-6.93 (m, 3H), 6.18 (d, J=8.8 Hz, 1H), 5.77 (s, 1H), 5.32 (t, J=6.8 Hz, 1H), 3.59 (s, 2H), 3.06 (br, 2H), 2.67 (s, 1H), 2.40 (s, 3H), 2.33 (s, 1H), 2.23 (s, 3H), 2.18 (s, 3H), 2.14 (s, 3H).

### Example 45

### N-((2,4-dimethylphenyl)(phenyl)methyl)-2-(2-(1-hydroxy-1-(pyridin-4-yl)ethyl)benzofuran-5-yl)acetamide

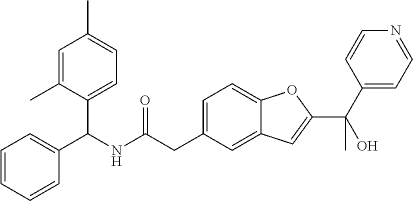 [SMILES: Cc1ccc(C(NC(=O)Cc2ccc3oc(C(C)(O)c4ccncc4)cc3c2)c2ccccc2)c(C)c1]

### a) methyl 2-(2-isonicotinoylbenzofuran-5-yl)acetate

K₂CO₃ (15.18 g, 110 mmol) was added to a solution of methyl 2-(3-formyl-4-hydroxyphenyl)acetate (9.7 g, 50 mmol) in DMF (160 mL) at rt. After stirring at rt for 30 min, 2-bromo-1-(pyridin-4-yl)ethanone hydrobromide (14.05 g, 50 mmol) was added to the mixture. The mixture was stirred at rt overnight, and water was added. The reaction mixture was extracted with EtOAc (250 mL×3), and the combined extracts were dried over Na₂SO₄, and concentrated under reduced pressure. The resulting crude product was purified by flash column (petroleum ether/EtOAc=1/1 to 1/1) to give 3.9 grams of title compound (yield: 26%). LCMSA024: 296.1 [M+H]+; Rt: 1.237 min

### b) 2-(2-isonicotinoylbenzofuran-5-yl)acetic acid

This compound was synthesized from methyl 2-(2-isonicotinoylbenzofuran-5-yl)acetate essentially as described in example 31 (b) (650 mg, 97.6%). LCMS-A027: 282.3 [M+H]+; Rt=1.10 min.

### c) N-((2,4-dimethylphenyl)(phenyl)methyl)-2-(2-isonicotinoylbenzofuran-5-yl)acetamide

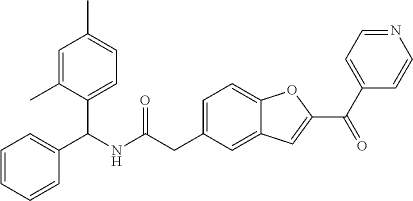 [SMILES: Cc1ccc(C(NC(=O)Cc2ccc3oc(C(=O)c4ccncc4)cc3c2)c2ccccc2)c(C)c1]

This compound was synthesized from 2-(2-isonicotinoylbenzofuran-5-yl)acetic acid and (2,4-dimethylphenyl)(phenyl)methanamine essentially as described in example 31 (b) (1.1 g, yield: 92.8%). LCMS-A036: 475.2 [M+H]⁺; Rt: 1.72 min.

### d) N-((2,4-dimethylphenyl)(phenyl)methyl)-2-(2-(1-hydroxy-1-(pyridin-4-yl)ethyl)benzofuran-5-yl)acetamide

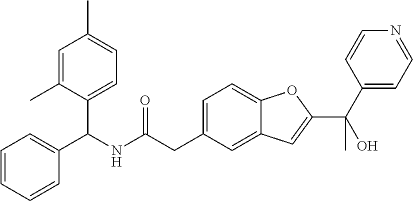 [SMILES: Cc1ccc(C(NC(=O)Cc2ccc3oc(C(C)(O)c4ccncc4)cc3c2)c2ccccc2)c(C)c1]

Methylmagnesium bromide (3M, 0.8 mL) was added to a solution of N-((2,4-dimethylphenyl)(phenyl)methyl)-2-(2-isonicotinoylbenzofuran-5-yl)acetamide (1.0 g, 2.1 mmol) in THF (30 mL) at 0° C. under nitrogen atmosphere. The mixture was stirred at rt for 4 h, then quenched with NH₄Cl (aqueous), and extracted with EtOAc (50 mL×3). The combined extracts were washed with brine, dried over sodium sulfate, and concentrated under reduced pressure. The resulting residue was purified by reverse phase HPLC using water/acetonitrile with 0.05% TFA to yield N-((2,4-dimethylphenyl)(phenyl)methyl)-2-(2-(1-hydroxy-1-(pyridin-4-yl)ethyl)benzofuran-5-yl)acetamide (0.9 g, 87.4%). LCMS-036: 491.2 [M+H]⁺; Rt: 1.41 min. ¹H NMR (CDCl₃, 400 MHz): δ 8.53 (s, 2H), 7.66 (s, 2H), 7.36-7.07 (m, 7H), 7.00-6.62 (m, 6H), 6.28 (d, J=8.0 Hz, 1H), 5.99 (d, J=7.6 Hz, 1H), 3.60 (s, 2H), 2.20 (s, 3H), 2.13 (s, 3H), 1.89 (s, 3H).

### Example 46

### 4-(1-(5-(2-(((2,4-dimethylphenyl)(phenyl)methyl)amino)-2-oxoethyl)benzofuran-2-yl)-1-hydroxyethyl)pyridine 1-oxide

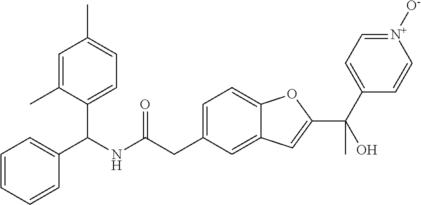 [SMILES: Cc1ccc(C(NC(=O)Cc2ccc3oc(C(C)(O)c4cc[n+]([O-])cc4)cc3c2)c2ccccc2)c(C)c1]

m-CPBA (175.4 mg, 1.02 mmol) was added to a solution of N-((2,4-dimethylphenyl)(phenyl)methyl)-2-(2-(1-hydroxy-1-(pyridin-4-yl)ethyl)benzofuran-5-yl)acetamide (500 mg, 1.02 mmol) in CH₂Cl₂ (20 mL). The reaction mixture was stirred at rt for 4 h, and then was extracted with CH₂Cl₂. The combined extracts were washed with brine, dried over sodium sulfate and concentrated under reduced pressure resulting in crude 4-(1-(5-(2-(((2,4-dimethylphenyl)(phenyl)methyl)amino)-2-oxoethyl)benzofuran-2-yl)-1-hydroxyethyl)pyridine 1-oxide (400 mg, 77.5%). Further purification afforded 4-(1-(5-(2-(((2,4-dimethylphenyl)(phenyl)methyl)amino)-2-oxoethyl)benzofuran-2-yl)-1-hydroxyethyl)pyridine 1-oxide (260 mg, 50% yield). LCMS-A020: 507.2 [M+H]⁺; Rt: 1.57 min. ¹H NMR (CDCL3, 400 MHz): δ 8.19 (d, J=6.0 Hz, 2H), 7.44-7.06 (m, 9H), 6.97-6.66 (m, 6H), 6.37 (d, J=8.0 Hz, 1H), 5.99 (d, J=7.6 Hz, 1H), 3.69 (s, 2H), 2.28 (s, 3H), 2.21 (s, 3H), 1.95 (s, 3H).

### Example 47

### N-((2,4-dimethylphenyl)(phenyl)methyl)-2-(2-(1-hydroxy-1-(2-methylpyridin-4-yl)ethyl)benzofuran-5-yl)acetamide

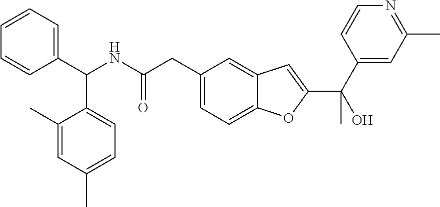 [SMILES: Cc1ccc(C(NC(=O)Cc2ccc3oc(C(C)(O)c4ccnc(C)c4)cc3c2)c2ccccc2)c(C)c1]

### a) N-methoxy-N,2-dimethylisonicotinamide

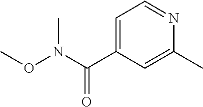

A mixture of 2-methylisonicotinic acid (582 mg, 4.25 mmol), EDCI (1.63 g, 8.5 mmol), HOBt (1.15 g, 8.5 mmol) and DIPEA (2.19 g, 17 mmol) in DMF (20 mL) was stirred at rt for 0.5 h. Then N,O-dimethylhydroxylamine hydrochloride (456 mg, 4.67 mmol) was added. After the addition, the mixture was stirred at rt overnight. Then the reaction solution was washed with water (20 mL×3) and brine (20 mL). The organic layer was dried over anhydrous sodium sulfate and filtered. The filtrate was concentrated and purified by silica gel column chromatography (petroleum ether/EtOAc=1/1) to obtain title compound (648 mg, yield: 85%) as pale yellow oil. LCMS-A024: 181.2 [M+H]⁺; Rₜ=0.729 min.

### b) 1-(2-methylpyridin-4-yl)ethanone

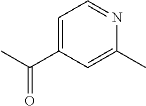

Methyl lithium (3M, 1.7 mL, 3.48 mmol) was added to a solution of N-methoxy-N,2-dimethylisonicotinamide (314 mg, 1.74 mmol) in dry THF (4 mL) at −78° C. under nitrogen protection. The mixture was stirred at −78° C. and slowly warm up to rt for 1.5 h. After completion, 10 mL of NH₄Cl solution (saturated) was added to the mixture, and it was extracted with EtOAc (10 mL×3). The combined organic layers were washed with brine (20 mL), dried over Na₂SO₄, and filtered. The filtrate was concentrated, and the residue was purified by silica gel column chromatography (CH₂Cl₂/MeOH=50/1) to obtain title compound (120 mg, yield: 51%). LCMS-A010: 136.1 [M+H]⁺; Rₜ=0.585 min

### c) 2-bromo-1-(2-methylpyridin-4-yl)ethanone hydrobromide

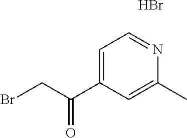

To a solution of 1-(2-methylpyridin-4-yl)ethanone (256 mg, 1.9 mmol) in hydrogen bromide-acetic acid solution (40%, 2 mL) at 0° C. was added a solution of Br₂ (303 mg, 1.9 mmol) in hydrogen bromide-acetic acid solution (40%, 0.5 mL) slowly. After the addition, the reaction mixture was stirred at 0° C. and was allowed to warm to rt overnight. After completion, the reaction mixture was concentrated to dryness, and the residue was washed with EtOAc (2 mL×3) to obtain title compound (444 mg, yield: 80%). LCMS-A010: 213.9 [M+H]⁺; Rₜ=1.484 min.

### d) methyl 2-(2-(2-methylisonicotinoyl)benzofuran-5-yl)acetate

This compound was synthesized from 2-bromo-1-(2-methylpyridin-4-yl)ethanone hydrochloride and methyl 2-(3-formyl-4-hydroxyphenyl)acetate essentially as described in example 45 (a) (370 mg, yield: 80%). LCMSA022: 310.1 [M+H]⁺; Rₜ=1.249 min.

### e) 2-(2-(2-methylisonicotinoyl)benzofuran-5-yl)acetic acid hydrochloride

This compound was synthesized from methyl 2-(2-(2-methylisonicotinoyl)benzofuran-5-yl)acetate essentially as described in example 31 (b) (397 mg, crude). LCMS-A024: 296.1 [M+H]⁺; Rₜ=0.947 min.

### f) N-((2,4-dimethylphenyl)(phenyl)methyl)-2-(2-(2-methylisonicotinoyl)benzofuran-5-yl)acetamide

This compound was synthesized from 2-(2-(2-methylisonicotinoyl)benzofuran-5-yl)acetic acid hydrochloride and (2,4-dimethylphenyl)(phenyl)methanamine hydrochloride essentially as described in example 12 (e) (560 mg, crude). LCMS-A024: 489.2[M+H]⁺; Rₜ=1.327 min.

### g) N-((2,4-dimethylphenyl)(phenyl)methyl)-2-(2-(1-hydroxy-1-(2-methylpyridin-4-yl)ethyl)benzofuran-5-yl)acetamide

This compound was synthesized from N-((2,4-dimethylphenyl)(phenyl)methyl)-2-(2-(2-methylisonicotinoyl)benzofuran-5-yl)acetamide essentially as described in example E (d) (6 mg, 19%). LCMS-A020: 505.3 [M+H]⁺; Rt: 1.69 min. ¹H NMR (MeOH-d₄, 400 MHz): δ 8.37 (d, J=5.6 Hz, 1H), 7.52 (d, J=1.2 Hz, 1H), 7.43 (s, 1H), 7.36 (d, J=8 Hz, 2H), 7.29-7.22 (m, 4H), 7.14 (d, J=7.6 Hz, 2H), 6.99-6.94 (m, 3H), 6.74 (d, J=0.8 Hz, 1H), 6.29 (s, 1H), 3.67 (s, 2H), 2.53 (s, 3H), 2.28 (s, 3H), 2.17 (s, 3H), 2.05 (s, 3H).

### Example 48

### N—((S)-(2,4-dimethylphenyl)(phenyl)methyl)-2-(2-(1-hydroxy-1-(3-methylpyridin-4-yl)ethyl)benzofuran-5-yl)acetamide

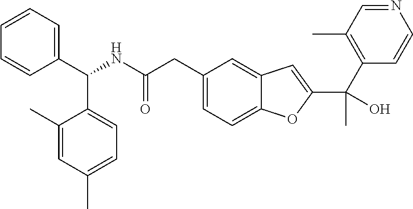 [SMILES: Cc1ccc([C@@H](NC(=O)Cc2ccc3oc(C(C)(O)c4ccncc4C)cc3c2)c2ccccc2)c(C)c1]

### a) 3-Methylisonicotinaldehyde

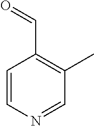

PdCl₂(dppf) CH₂Cl₂ (220 mg, 0.27 mmol) and K₂CO₃ (745 mg, 5.4 mmol) were added to a solution of 3-chloroisonicotinaldehyde (382 mg, 2.7 mmol) and 2,4,6-trimethyl-1,3,5,2,4,6-trioxatriborinane (680 mg, 5.4 mmol) in 5 mL of dioxane and 0.6 mL of water, and the mixture was stirred at 100° C. for 3 h. At that point, solvent was removed under reduced pressure, and water (15 mL) was added to the mixture. The mixture was then extracted with EtOAc (3×15 mL). The combined organic extracts were washed with brine (30 mL), dried over Na₂SO₄, and filtered. The filtrate was concentrated unde reduced pressure to obtain crude 3-methylisonicotinaldehyde (680 mg, crude) as a dark brown solid, which was carried through without further purification. LCMSA012: 122.1 [M+H]⁺; Rₜ=0.906 min; Purity: 30.1% (254 nm).

### b) 1-(3-Methylpyridin-4-yl)ethanol

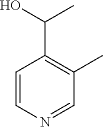

MeMgBr (3M, 2 mL, 5.95 mmol) was added to a solution of crude 3-methylisonicotinaldehyde (600 mg, 4.96 mmol) in 6 mL of dry THF at 0° C. (NaCl/ice bath) under N₂. The resulted mixture was then allowed to warm up to rt and stirred for 2 h. A NH₄Cl solution (sat., 10 mL) was then added to the mixture. The mixture was then extracted with EtOAc (3×10 mL). The combined organic extracts were washed with brine (20 mL), dried over Na₂SO₄, and filtered. The filtrate was concentrated under reduced pressure to obtain crude 1-(3-methylpyridin-4-yl)ethanol as brown oil (615 mg, crude), which was carried through without further purification. LCMSA012: 138.1 [M+H]⁺; Rₜ=0.860 min; Purity: 56.2% (254 nm).

### c) 1-(3-Methylpyridin-4-yl)ethanone

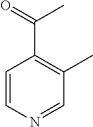

Manganese(IV) oxide (1.35 g, 15.5 mol) was added to a solution of 1-(3-methylpyridin-4-yl)ethanol (530 mg, 3.87 mol) in CH₂Cl₂ (5 mL). The mixture was stirred at 50° C. overnight, the cooled to rt and filtered. The filtrate was concentrated under reduced pressure and the resulting residue was purified with silica gel column chromatography (CH₂Cl₂/MeOH=50/1) to obtain title compound (115 g, 22%). LCMSA022: 136.2 [M+H]⁺; Rₜ=0.795 min; Purity=41.5% (214 nm).

### d) 2-Bromo-1-(3-methylpyridin-4-yl)ethanone

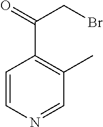

To a solution of 1-(3-methylpyridin-4-yl)ethanone (115 mg, 0.85 mmol) in hydrogen bromide-acetic acid solution (40%, 1.5 mL) at 0° C. was added a solution of Br₂ (136 mg, 0.85 mmol) in hydrogen bromide-acetic acid solution (40%, 1 mL) was added slowly. After the addition, the reaction mixture was stirred at 0° C. and then the ice was allowed to melt over 7 h. The reaction mixture was concentrated under reduced pressure, and the residue was washed with EtOAc (2 mL×3) to obtain title compound (157 mg, yield: 87%), which was carried through without further purification. LCMS010: 216.0 [M+H]⁺; Rₜ=1.203 min; Purity: 71.5% (214 nm)

### e) Methyl 2-(2-(3-methylisonicotinoyl)benzofuran-5-yl)acetate

This compound was synthesized from 2-bromo-1-(3-methylpyridin-4-yl)ethanone essentially as described in Example 45 (a) (89 mg, yield: 54%). LCMSA024: 310.1 [M+H]⁺; Rₜ=1.096 min.

### f) 2-(2-(3-Methylisonicotinoyl)benzofuran-5-yl)acetic acid

This compound was synthesized from methyl 2-(2-(3-methylisonicotinoyl)benzofuran-5-yl)acetate essentially as described in example 31 (b) (95 mg, yield: 100%) and was used as such for the next step. LCMS022: 296.1 [M+H]⁺; Rₜ=1.112 min.

### g) (S)-(2,4-dimethylphenyl)(phenyl)methanamine

(2,4-dimethylphenyl)(phenyl)methanamine (139 g) was resolved using the following method:

Instrument: Thar SFC Prep 200 (Thar Technologies, Waters); Column: ChiralPak AD-H, 50 mm I.D.×250 mm Length, 5 μm (Daicel Chemical Industries Co., Ltd); Column Temperature: 35° C.; Mobile Phase: CO2/MeOH/DEA=50/50/0.5; Flow rate: 120 g/min; Back Pressure: 100 Bar; Wavelength: 214 nm; Cycle time: 3.3 min; Injection Volume: 3.0 mL; Load per injection: 90 mg; Feed solution: 3000 mg dissolved in 100 mL MeOH.

Said resolution yielded 2 enantiomers:
Peak 1, 55 g, RT: 3.9 min, 100% ee;
Peak 2, 50 g, RT: 5.0 min, 100% ee.
   Stereochemistry of peak 1 and peak 2 was assigned by VCD:
peak 1: (R)-(2,4-dimethylphenyl)(phenyl)methanamine
peak 2: (S)-(2,4-dimethylphenyl)(phenyl)methanamine

Alternatively (S)-(2,4-dimethylphenyl)(phenyl)methanamine can also be obtained by recrystallization:

D-DBTA.H₂O (dibenzoyl-D-tartaric acid monohydrate) (101.8 g, 284 mmol) was added to a mixture of MeOH (2.44 L) and H₂O (0.6 L). The mixture was heated until all the solid was dissolved. Then (2,4-dimethylphenyl)(phenyl)methanamine (R/S=1/1) (60 g, 284 mmol) was added drop-wise. After stirring at 65° C. for 3 h, the mixture was slowly cooled to rt slowly and stirred at rt overnight. The solid was collected by filtration and dried under reduced pressure to afford 81 grams of white solid. This white solid was resuspended in MeOH (3.24 L) and heated to 65° C. until all the solid disappeared. Then H₂O (0.81 L) was added. Then the mixture was stirred at 65° C. for 20 mins and then allowed to cool to rt slowly and stirred at rt overnight. The solid was collected and dried under reduced pressure to give 18.8 grams of (S)-(2,4-dimethylphenyl)(phenyl)methanamine-dibenzoyl-D-tartaric acid salt as a white solid (yield: 12% in 2 steps, 97% ee). LC-MS036: 195.1 [M−NH₂]⁺; Rt: 1.247 min

### h) (S)—N-((2,4-dimethylphenyl)(phenyl)methyl)-2-(2-(3-methylisonicotinoyl)benzofuran-5-yl)acetamide

This compound was synthesized from 2-(2-(3-methylisonicotinoyl)benzofuran-5-yl)acetic acid and (S)-(2,4-dimethylphenyl)(phenyl)methanamine hydrochloride essentially as described in example 2 (b) (148 mg, crude). LCMS-024: 489.2[M+H]⁺; Rt=1.347 min.

### i) N—((S)-(2,4-dimethylphenyl)(phenyl)methyl)-2-(2-(1-hydroxy-1-(3-methylpyridin-4-yl)ethyl)benzofuran-5-yl)acetamide

This compound was synthesized from (S)—N-((2,4-dimethylphenyl)(phenyl)methyl)-2-(2-(3-methylisonicotinoyl)benzofuran-5-yl)acetamide essentially as described in Example 45 (d) (15 mg, yield: 18%). LCMS-A020: 505.7[M+H]⁺; Rt=1.69 min. ¹H NMR (CDCl₃, 400 MHz): δ 8.48 (d, J=5.6 Hz, 1H), 8.36 (s, 1H), 7.64 (d, J=5.2 Hz, 1H), 747 (s, 1H), 7.37 (d, J=8.4 Hz, 1H), 7.26-7.18 (m, 5H), 7.06 (d, J=7.2 Hz, 1H), 6.97 (s, 1H), 6.88 (d, J=7.6 Hz, 1H), 6.67 (d, J=7.6 Hz, 1H), 6.67 (s, 1H), 6.36 (d, J=8.0 Hz, 1H), 5.62 (d, J=8.0, 1H), 3.70 (s, 2H), 2.28 (s, 3H), 2.20 (s, 3H), 2.15 (s, 3H), 2.00 (s, 3H).

### Example 49

### 2-(2-((3-aminoazetidin-1-yl)(3-methylpyridin-4-yl)methyl)benzofuran-5-yl)-N—((S)-(2,4-dimethylphenyl)(phenyl)methyl)acetamide

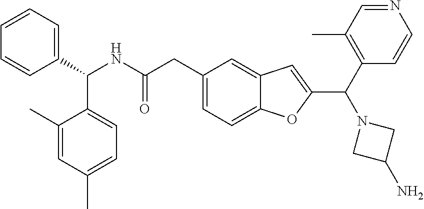 [SMILES: Cc1ccc([C@@H](NC(=O)Cc2ccc3oc(C(c4ccncc4C)N4CC(N)C4)cc3c2)c2ccccc2)c(C)c1]

a) N—((S)-(2,4-dimethylphenyl)(phenyl)methyl)-2-(2-(hydroxy(3-methylpyridin-4-yl)methyl)benzofuran-5-yl)acetamide

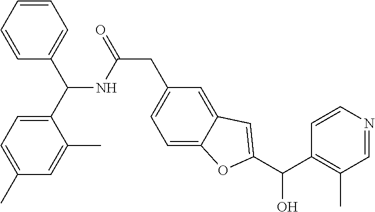 [SMILES: Cc1ccc(C(NC(=O)Cc2ccc3oc(C(O)c4ccncc4C)cc3c2)c2ccccc2)c(C)c1]

This compound was synthesized from (S)—N-((2,4-dimethylphenyl)(phenyl)methyl)-2-(2-(3-methylisonicotinoyl)benzofuran-5-yl)acetamide essentially as described in example 12 (a) (38 mg, yield: 26%). LCMS-A024: 491.2 [M+H]⁺; Rt=1.197 min.

### b) 2-(2-(chloro(3-methylpyridin-4-yl)methyl)benzofuran-5-yl)-N—((S)-(2,4-dimethylphenyl)(phenyl)methyl)acetamide

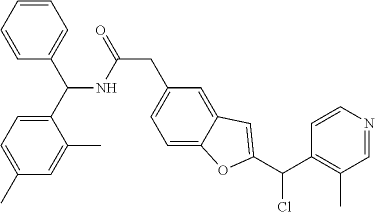 [SMILES: Cc1ccc(C(NC(=O)Cc2ccc3oc(C(Cl)c4ccncc4C)cc3c2)c2ccccc2)c(C)c1]

This compound was synthesized from N—((S)-(2,4-dimethylphenyl)(phenyl)methyl)-2-(2-(hydroxy(3-methylpyridin-4-yl)methyl)benzofuran-5-yl)acetamide essentially as described in example 28 (a) (120 mg, 77%).

### c) tert-butyl (1-((5-(2-(((S)-(2,4-dimethylphenyl)(phenyl)methyl)amino)-2-oxoethyl)benzofuran-2-yl)(3-methylpyridin-4-yl)methyl)azetidin-3-yl)carbamate

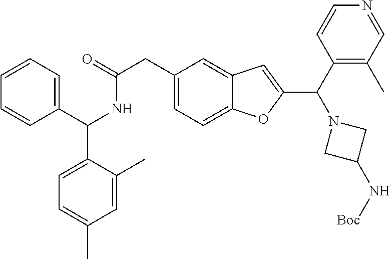 [SMILES: Cc1ccc(C(NC(=O)Cc2ccc3oc(C(c4ccncc4C)N4CC(NC(=O)OC(C)(C)C)C4)cc3c2)c2ccccc2)c(C)c1]

This compound was synthesized from 2-(2-(chloro(3-methylpyridin-4-yl)methyl)benzofuran-5-yl)-N—((S)-(2,4-dimethylphenyl)(phenyl)methyl)acetamide essentially as described in example 39 (e) (50 mg, yield 49.5%). LCMS-A012: 645.7[M+H]⁺; Rt=1.79 min.

### d) 2-(2-((3-aminoazetidin-1-yl)(3-methylpyridin-4-yl)methyl)benzofuran-5-yl)-N—((S)-(2,4-dimethylphenyl)(phenyl)methyl)acetamide

This compound was synthesized from tert-butyl (1-((5-(2-(((S)-(2,4-dimethylphenyl)(phenyl)methyl)amino)-2-oxoethyl)benzofuran-2-yl)(3-methylpyridin-4-yl)methyl)azetidin-3-yl)carbamate essentially as described in example 39 (f) (10 mg, yield 23%). LCMSA020: 545.7[M+H]⁺; Rt=1.61 min. ¹H NMR (DMSO, 400 MHz): δ 8.87 (d, J=8.0 Hz, 1H), 8.62 (d, J=4.4 Hz, 1H), 8.54 (s, 1H), 7.47 (s, 1H), 7.38 (d, J=8.0 Hz, 1H), 7.31-7.14 (m, 7H), 7.01-6.90 (m, 4H), 6.16 (d, J=8.4 Hz, 1H), 5.12 (s, 1H), 3.88-3.83 (m, 1H), 3.59-3.54 (m, 3H), 3.52-3.48 (m, 1H), 3.37-3.34 (m, 1H), 3.17-3.14 (m, 1H), 2.37 (s, 3H), 2.22 (s, 3H), 2.14 (s, 3H).

### Example 50

### 2-(2-((3,5-dimethylisoxazol-4-yl)methyl)-1H-benzo[d]imidazol-5-yl)-N-((2,4-dimethylphenyl)(phenyl)methyl)acetamide

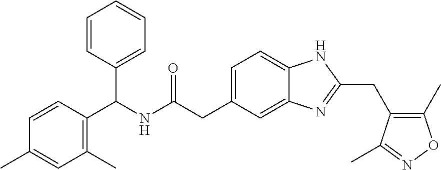

### a) 4-(2,2-dibromovinyl)-3,5-dimethylisoxazole

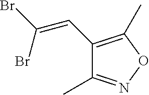

This compound was synthesized from 3,5-dimethylisoxazole-4-carbaldehyde essentially as described in example 1 (c).

### b) Methyl 2-(2-((3,5-dimethylisoxazol-4-yl)methyl)-1H-benzo[d]imidazol-6-yl)acetate

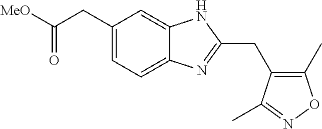 [SMILES: COC(=O)Cc1ccc2nc(Cc3c(C)noc3C)[nH]c2c1]

Methyl 2-(3,4-diaminophenyl)acetate (300 mg, 1.66 mmol), 4-(2,2-dibromovinyl)-3,5-dimethylisoxazole (crude), and 1,8-diazabicycloundec-7-ene (0.9 g, 8.3 mmol) in methylpyrrolidone (3 mL) were heated overnight at 100° C. The reaction mixture was diluted with ethyl acetate and water was added. The organic layer was dried over sodium sulfate, and concentrated under reduced pressure. The resulting residue was purified using silica gel chromatography (0-100% EtOAc/Hexanes) to give title compound (0.1 g, 25%). LCMS-T1: 300.1 [M+H]⁺; Rt: 3.49 min

### c) 2-(2-((3,5-dimethylisoxazol-4-yl)methyl)-1H-benzo[d]imidazol-5-yl)-N-((2,4-dimethylphenyl)(phenyl)methyl)acetamide

This compound was synthesized from methyl 2-(2-((3,5-dimethylisoxazol-4-yl)methyl)-1H-benzo[d]imidazol-6-yl)acetate and (2,4-dimethylphenyl)(phenyl)methanamine essentially as described in Example 45 (b) and (c) to give title compound (12 mg). LCMS-T1: 479.4 [M+H]⁺; Rt: 5.42 min ¹H NMR (CDCl₃, 400 MHz): 7.48 (d, J=11 Hz, 1H), 7.30-7.15 (m, 6H), 7.10-6.94 (m, 4H), 6.32 (s, 1H), 3.63 (s, 2H), 3.11 (s, 2H), 2.33 (s, 3H), 2.24 (s, 6H), 2.20 (s, 3H).

### Example 51

### 2-(2-((3,5-dimethylisoxazol-4-yl)methyl)benzo[d]oxazol-5-yl)-N-((2,4-dimethylphenyl)(phenyl)methyl)acetamide

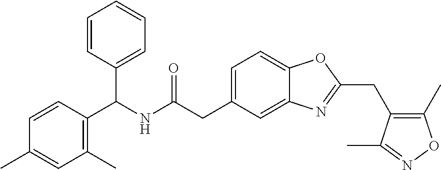

This compound was synthesized from 1-(3-amino-4-hydroxyphenyl)-4-(2,4-dimethylphenyl)-4-phenylbutan-2-one and 4-(2,2-dibromovinyl)-3,5-dimethylisoxazole essentially as described in example 50 (b) (8 mg, 2%). LCMS-T1: 480.1 [M+H]⁺; Rt: 6.34 min. ¹H NMR (CDCl₃, 400 MHz): 7.56-7.42 (m, 3H), 7.26-7.19 (m, 5H), 7.04-6.86 (m, 2H), 6.74 (d, J=7.6 Hz, 1H), 6.33 (d, J=7.5 Hz, 1H), 5.92 (s, 1H), 3.95 (s, 2H), 3.72 (s, 2H), 2.40 (s, 3H), 2.26 (s, 6H), 2.18 (s, 3H).

### Example 52

### N-((4-chloro-2-methylphenyl)(phenyl)methyl)-2-(2-(2,4-dimethylpyridin-3-yl)benzo[d]oxazol-5-yl)acetamide

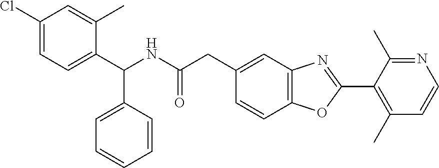 [SMILES: Cc1cc(Cl)ccc1C(NC(=O)Cc1ccc2oc(-c3c(C)ccnc3C)nc2c1)c1ccccc1]

### a) N-(5-(2-(((4-chloro-2-methylphenyl)(phenyl)methyl)amino)-2-oxoethyl)-2-hydroxyphenyl)-2,4-dimethylnicotinamide

This compound was synthesized from 2,4-dimethylnicotinic acid and 2-(3-amino-4-hydroxyphenyl)-N-((4-chloro-2-methylphenyl)(phenyl)methyl)acetamide essentially as described in example 18 (c) (320 mg, 62.4%). LC-MS: m/z 514 [M+H]⁺; Rt=1.525 min.

### b) N-((4-chloro-2-methylphenyl)(phenyl)methyl)-2-(2-(2,4-dimethylpyridin-3-yl)benzo[d]oxazol-5-yl)acetamide

This compound was synthesized from N-(5-(2-(((4-chloro-2-methylphenyl)(phenyl)methyl)amino)-2-oxoethyl)-2-hydroxyphenyl)-2,4-dimethylnicotinamide essentially as described in example 18 (d) (15 mg). LC-MS: 496 [M+H]⁺; Rt=1.546 min ¹H NMR (DMSO, 500 MHz): δ 9.06 (d, J=8.0 Hz, 1H), 8.53 (d, J=5.0 Hz, 1H), 7.74 (d, J=7.5 Hz, 2H), 7.40-7.21 (m, 7H), 7.20-6.20 (m, 5H), 6.21 (d, J=8.0 Hz, 1H), 3.71 (s, 2H), 2.46 (s, 3H), 2.31 (s, 3H), 2.21 (s, 3H).

### Example 53

### N-((4-chloro-2-methylphenyl)(phenyl)methyl)-2-(2-(2-phenylpyridin-3-yl)benzofuran-5-yl)acetamide

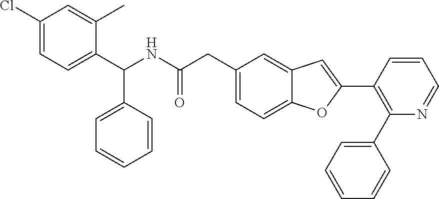 [SMILES: Cc1cc(Cl)ccc1C(NC(=O)Cc1ccc2oc(-c3cccnc3-c3ccccc3)cc2c1)c1ccccc1]

### a) 2-phenyl-3-(4,4,5,5-tetramethyl-1,3,2-dioxaborolan-2-yl)pyridine

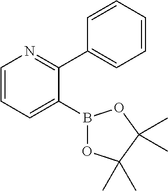 [SMILES: CC1(C)OB(c2cccnc2-c2ccccc2)OC1(C)C]

Bis(pinacolato)diboron (284 mg, 1.12 mmol), Pd(dppf)Cl₂ (191 mg, 0.26 mmol), and KOAc (253 mg, 2.58 mmol) were added to a solution of 3-bromo-2-phenylpyridine (200 mg, 0.86 mmol) in dioxane (20 mL) under N₂. The resulting mixture was refluxed for 2 h. After cooling to rt, water (40 mL) was added and the resulting mixture was extracted with EtOAc (30 mL×3). The combined extracts were washed with brine (10 mL×3), dried over Na₂SO₄, and concentrated under reduced pressure. The resulting residue was purified by Pre-TLC with EtOAc/petroleum ether: ½ to obtain 2-phenyl-3-(4,4,5,5-tetramethyl-1,3,2-dioxaborolan-2-yl)pyridine (68 mg, yield: 28%) as yellow solid. LC-MS: 282 [M+H]⁺; Rt=1.444 min.

### b) N-((4-chloro-2-methylphenyl)(phenyl)methyl)-2-(2-(2-phenylpyridin-3-yl)benzofuran-5-yl)acetamide

This compound was synthesized from 2-phenyl-3-(4,4,5,5-tetramethyl-1,3,2-dioxaborolan-2-yl)pyridine and 2-(2-bromobenzofuran-5-yl)-N-((4-chloro-2-methylphenyl)(phenyl)methyl)acetamide essentially as described in example 11 (c) (5 mg, yield: 9%). LC-MS: 544 [M+H]⁺; Rt=1.810 min. ¹H NMR (DMSO-d6, 500 MHz): δ 8.96 (d, J=8.4 Hz, 1H), 8.71-8.70 (m, 1H), 8.26-8.24 (m, 1H), 8.57-8.54 (m, 1H), 7.43-7.08 (m, 16H), 6.46 (s, 1H), 6.18 (d, J=8.4 Hz, 1H), 3.56 (s, 2H), 2.17 (s, 3H).

### Examples 54 and 55

### N-((2,4-dimethylphenyl)(phenyl)methyl)-2-(2-(2-methylpyridin-3-yl)-1,2,3,4-tetrahydroisoquinolin-6-yl)acetamide and N-((2,4-dimethylphenyl)(phenyl)methyl)-2-(2-(2-methylpyridin-3-yl)-1,2,3,4-tetrahydroisoquinolin-8-yl)acetamide

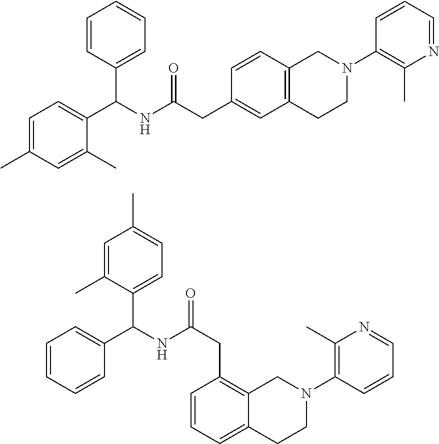

### a) 2-(3-bromophenyl)ethanamine

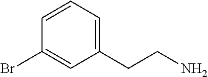

Concentrated H₂SO₄ (2.5 g, 25.5 mmol) was added slowly to a solution of LiAlH₄ (3.1 g, 81.6 mmol) in dry THF (100 mL) at 0° C. After stirring at 0° C. for 1 h, a solution of 2-(3-bromophenyl)acetonitrile (10 g, 51 mmol) in THF was added drop-wise. The mixture was allowed to warm up to rt and stirred at rt for 1 h, then cooled back to 0° C. and quenched by the addition of a 1:1 THF:H₂O mixture (12.4 mL), followed by a 3.6 M solution of NaOH (24.4 mL). The solid was removed by filtration, and the filtrate was concentrated under reduced pressure to give 2-(3-bromophenyl)ethanamine as a yellow oil (10.1 g, yield 98.1%), which was carried through without further purification. LC-MS (011): 202.7 [M+H]⁺; Rt: 1.64 min.

### b) N-(3-bromophenethyl)-2,2,2-trifluoroacetamide

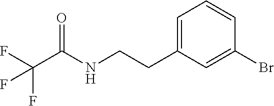 [SMILES: O=C(NCCc1cccc(Br)c1)C(F)(F)F]

2,6-Lutidine (6.4 mL, 55.0 mmol) was added to a solution of 2-(3-bromophenyl)ethanamine (10 g, 50.0 mmol) in CH₂Cl₂ (250 mL), followed by addition of (CF₃CO)₂O (7.8 mL 55.0 mmol), and the reaction mixture was stirred overnight. NaHCO₃ (sat) solution (100 mL) was then added and the mixture was extracted with EtOAc (3×80 mL). The combined extracts were collected and concentrated under reduced pressure. The residue was recrystallizd by petroleum ether to obtain N-(3-bromophenethyl)-2,2,2-trifluoroacetamide (7.8 g, yield 52.7%) as a white solid. LC-MS (024): 298.7 [M+H]⁺; Rt: 1.71 min

### c) 1-(6-bromo-3,4-dihydroisoquinolin-2(1H)-yl)-2,2,2-trifluoroethanone and 1-(8-bromo-3,4-dihydroisoquinolin-2(1H)-yl)-2,2,2-trifluoro ethanone

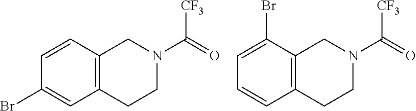

Concentrated H₂SO₄ (8.8 mL 33.2 mmol) and (CH₂O) (894 mg, 29.8 mmol) were added to a solution of N-(3-bromophenethyl)-2,2,2-trifluoroacetamide (6.3 g, 21.3 mmol) in AcOH (23 mL) under N₂ at 0° C. The mixture was allowed to warm up to rt and stirred overnight. The mixture was then poured into ice-water and extracted with EtOAc (3×40 mL). The combined extracts were concentrated under reduced pressure to get the desired white solid as a mixture 1-(6-bromo-3,4-dihydroisoquinolin-2(1H)-yl)-2,2,2-trifluoroethanone and 1-(8-bromo-3,4-dihydroisoquinolin-2(1H)-yl)-2,2,2-trifluoroethanone (6.0 g, yield 91.6%), which was used without further purification. LC-MS (024): 310.7 [M+H]⁺; Rt: 2.09 min

### d) 2,2,2-trifluoro-1-(6-(4,4,5,5-tetramethyl-1,3,2-dioxaborolan-2-yl)-3,4-dihydroisoquinolin-2(1H)-yl)ethanone and 2,2,2-trifluoro-1-(8-(4,4,5,5-tetramethyl-1,3,2-dioxaborolan-2-yl)-3,4-dihydroisoquinolin-2(1H)-yl)ethanone

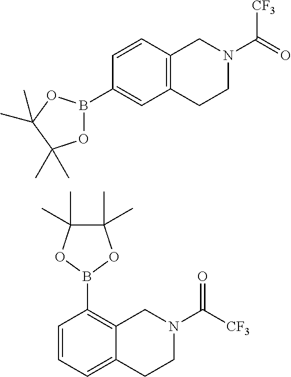

These compounds were synthesized from 1-(6-bromo-3,4-dihydroisoquinolin-2(1H)-yl)-2,2,2-trifluoroethanone and 1-(8-bromo-3,4-dihydroisoquinolin-2(1H)-yl)-2,2,2-trifluoroethanone essentially as described in example 53 (a) (11.20 g, 74.7%) as white product. LC-MS (022): 308.97 [M+H]⁺; Rt: 1.723 min

### e) ethyl 2-(2-(2,2,2-trifluoroacetyl)-1,2,3,4-tetrahydroisoquinolin-6-yl)acetate and ethyl 2-(2-(2,2,2-trifluoroacetyl)-1,2,3,4-tetrahydroisoquinolin-8-yl)acetate

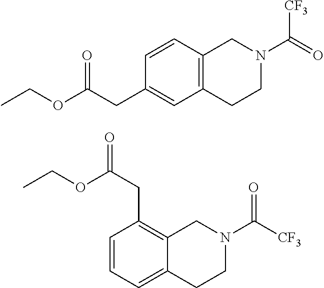

These compounds were synthesized from 2,2,2-trifluoro-1-(6-(4,4,5,5-tetramethyl-1,3,2-dioxab oro lan-2-yl)-3,4-dihydroisoquinolin-2(1H)-yl)ethanone and 2,2,2-trifluoro-1-(8-(4,4,5,5-tetramethyl-1,3,2-dioxaborolan-2-yl)-3,4-dihydroisoquinolin-2(1H)-yl)ethanone essentially as described in example 11 (c) (2.70 g, 57.2%) as colorless liquid. LC-MS (022): 315.7 [M+H]⁺; Rt: 1.54 min

### f) ethyl 2-(1,2,3,4-tetrahydroisoquinolin-6-yl)acetate and ethyl 2-(1,2,3,4-tetrahydroisoquinolin-8-yl)acetate

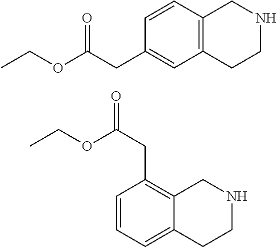

K₂CO₃ (5.93 g, 42.95 mmol) was added to a solution of compound ethyl 2-(2-(2,2,2-trifluoroacetyl)-1,2,3,4-tetrahydroisoquinolin-6-yl)acetate and ethyl 2-(2-(2,2,2-trifluoroacetyl)-1,2,3,4-tetrahydroisoquinolin-8-yl)acetate (2.71 g, 8.59 mmol) in EtOH/H₂O (80 mL, 95%). The mixture was stirred overnight at rt, and then extracted with EtOAc (3×80 mL). The combined extracts were dried over Na₂SO₄, and concentrated under reduced pressure. The resulting residue was purified by P-HPLC to get ethyl 2-(1,2,3,4-tetrahydroisoquinolin-6-yl)acetate and ethyl 2-(1,2,3,4-tetrahydroisoquinolin-8-yl)acetate (752 mg, 40%) as a colourless liquid. LC-MS (010): 219.7 [M+H]⁺; Rt: 1.45 min

### g) ethyl 2-(2-(2-methylpyridin-3-yl)-1,2,3,4-tetrahydroisoquinolin-6-yl)acetate and ethyl 2-(2-(2-methylpyridin-3-yl)-1,2,3,4-tetrahydroisoquinolin-8-yl)acetate

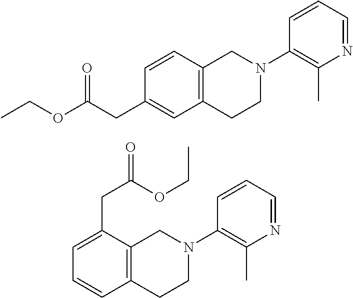

3-Bromo-2-methylpyridine (454 mg, 1.69 mmol), x-phos (73 mg, 0.153 mmol), Pd(dba)₂ (74 mg, 75 mmol), and Cs₂CO₃ (1.49 g, 4.6 mmol) were added to a solution of ethyl 2-(1,2,3,4-tetrahydroisoquinolin-6-yl)acetate and ethyl 2-(1,2,3,4-tetrahydroisoquinolin-8-yl)acetate (336 mg, 1.53 mmol) in toluene (40 mL). The mixture was degassed and recharged with N₂, then stirred overnight at 80° C. The reaction mixture was then extracted with EtOAc (3×50 mL). The combined extracts were washed with water (100 mL), dried over Na₂SO₄, and concentrated under reduced pressure. The resulting residue was purified by P-HPLC to obtain ethyl 2-(2-(2-methylpyridin-3-yl)-1,2,3,4-tetrahydroisoquinolin-6-yl)acetate and ethyl 2-(2-(2-methylpyridin-3-yl)-1,2,3,4-tetrahydroisoquinolin-8-yl)acetate (106 mg, 16.7%) as a white solid. LC-MS (024): 310.7 [M+H]⁺; Rt=1.07 min. Purity: 100% (254 nm).

### h) 2-(2-(2-methylpyridin-3-yl)-1,2,3,4-tetrahydroisoquinolin-6-yl)acetic acid and 2-(2-(2-methylpyridin-3-yl)-1,2,3,4-tetrahydroisoquinolin-8-yl)acetic acid

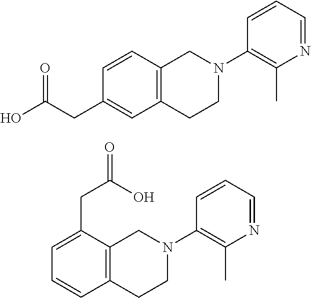

These compounds were synthesized from ethyl 2-(2-(2-methylpyridin-3-yl)-1,2,3,4-tetrahydroisoquinolin-6-yl)acetate and ethyl 2-(2-(2-methylpyridin-3-yl)-1,2,3,4-tetrahydroisoquinolin-8-yl)acetate essentially as described in example 11 (c) (103 mg) as solid. LC-MS (024): 282.7 [M+H]⁺; Rt=0.91 min.

### i) N-((2,4-dimethylphenyl)(phenyl)methyl)-2-(2-(2-methylpyridin-3-yl)-1,2,3,4-tetrahydroisoquinolin-8-yl)acetamide and N-((2,4-dimethylphenyl)(phenyl)methyl)-2-(2-(2-methylpyridin-3-yl)-1,2,3,4-tetrahydroisoquinolin-6-yl)acetamide

These compounds were synthesized from 2-(2-(2-methylpyridin-3-yl)-1,2,3,4-tetrahydroisoquinolin-6-yl)acetic acid and 2-(2-(2-methylpyridin-3-yl)-1,2,3,4-tetrahydroisoquinolin-8-yl)acetic acid and (2,4-dimethylphenyl)(phenyl)methanamine essentially as described in example 2 (b):

N-((2,4-dimethylphenyl)(phenyl)methyl)-2-(2-(2-methylpyridin-3-yl)-1,2,3,4-tetrahydroisoquinolin-8-yl)acetamide: (2 mg, yield: 1.2%); LC-MS (024): 476.7 [M+H]⁺; Rt: 1.583 min. ¹H NMR (MeOD, 400 MHz): δ 8.27-8.26 (d, J=5.6 Hz, 1H), 7.98-7.96 (d, J=8.4 Hz, 1H), 7.66-7.62 (m, 1H), 7.28-7.24 (m, 3H), 7.21-7.10 (m, 5H), 6.98-6.96 (m, 2H), 6.89-6.87 (d, J=8.0 Hz, 1H), 6.30 (s, 1H), 4.36-4.32 (m, 2H), 3.65 (s, 2H), 3.45-3.42 (m, 2H), 2.98-2.95 (m, 2H), 2.72 (s, 3H), 2.25 (s, 3H), 2.17 (s, 3H).

N-((2,4-dimethylphenyl)(phenyl)methyl)-2-(2-(2-methylpyridin-3-yl)-1,2,3,4-tetrahydroisoquinolin-6-yl)acetamide: (4 mg, yield: 2.3%); LC-MS (024): 476.7 [M+H]⁺; Rt=1.546 min. ¹H NMR (MeOD, 400 MHz): δ 8.32-8.31 (d, J=5.6 Hz, 1H), 8.21-8.19 (d, J=8.4 Hz, 1H), 7.82-7.79 (m, 1H), 7.33-7.25 (m, 3H), 7.17-7.11 (m, 5H), 7.00-6.96 (m, 3H), 6.31 (s, 1H), 6.58 (s, 1H), 4.32 (s, 2H), 3.57 (s, 2H), 3.45-3.42 (m, 2H), 3.02-2.99 (m, 2H), 2.77 (s, 3H), 2.28 (s, 3H), 2.19 (s, 3H).

### Example 56

### 2-(2-(1-(3,5-dimethylisoxazol-4-yl)-1-hydroxyethyl)benzofuran-5-yl)-N-((2,4-dimethylphenyl)(phenyl)methyl)acetamide

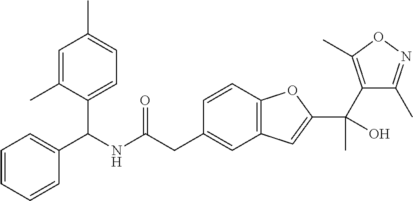 [SMILES: Cc1ccc(C(NC(=O)Cc2ccc3oc(C(C)(O)c4c(C)noc4C)cc3c2)c2ccccc2)c(C)c1]

This compound was synthesized from 2-(2-(3,5-dimethylisoxazole-4-carbonyl)benzofuran-5-yl)-N-((2,4-dimethylphenyl)(phenyl)methyl)acetamide essentially as described in Example 45 (d) (15 mg, yield: 24%). LC-MS: 509 [M+H]⁺; Rt=1.705 min. ¹H NMR (CDCl₃, 500 MHz): δ 7.39-7.33 (m, 2H), 7.19-7.11 (m, 5H), 6.99-6.81 (m, 4H), 6.69-5.90 (m, 4H), 3.63 (s, 2H), 2.20 (s, 3H), 2.19 (s, 3H), 2.16 (s, 3H), 2.12 (s, 3H), 1.87 (s, 3H).

### Example 57

### 5-(5-(2-(((4-chloro-2-methylphenyl)(phenyl)methyl)amino)-2-oxoethyl)benzofuran-2-yl)nicotinic acid

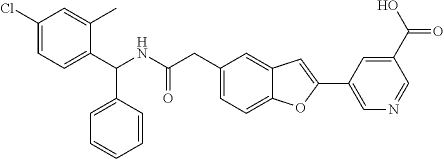 [SMILES: Cc1cc(Cl)ccc1C(NC(=O)Cc1ccc2oc(-c3cncc(C(=O)O)c3)cc2c1)c1ccccc1]

### a) ethyl 5-(5-(2-(((4-chloro-2-methylphenyl)(phenyl)methyl)amino)-2-oxoethyl)benzofuran-2-yl)nicotinate

This compound was synthesized from 2-(2-bromobenzofuran-5-yl)-N4(4-chloro-2-methylphenyl)(phenyl)methyl)acetamide and ethyl 5-(4,4,5,5-tetramethyl-1,3,2-dioxaborolan-2-yl)nicotinate essentially as described in example 11 (c) (70 mg, yield: 62%). LC-MS: 539 [M+H]⁺; Rt=1.872 min

### b) 5-(5-(2-(((4-chloro-2-methylphenyl)(phenyl)methyl)amino)-2-oxoethyl)benzofuran-2-yl)nicotinic acid

This compound was synthesized from ethyl 5-(5-(2-(((4-chloro-2-methylphenyl)(phenyl)methyl)amino)-2-oxoethyl)benzofuran-2-yl)nicotinate essentially as described in example 31 (b) (19 mg, yield 29%). LC-MS: 510.9 [M+H]⁺; Rt=1.673 min. ¹H NMR (DMSO-d₆, 400 MHz): δ 9.33 (d, J=2.0 Hz, 1H), 9.04-8.99 (m, 2H), 8.65-8.64 (m, 1H), 7.73 (s, 1H), 7.61-7.58 (m, 2H), 7.35-7.11 (m, 10H), 6.20 (d, J=8.0 Hz, 1H), 3.63 (s, 2H), 2.18 (s, 3H).

### Example 58

### 2-(2-(1-(3,5-dimethylisoxazol-4-yl)ethyl)benzofuran-5-yl)-N-((2,4-dimethylphenyl)(phenyl)methyl)acetamide

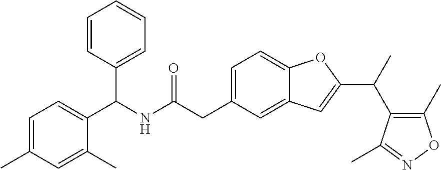

### a) 2-(2-(1-(3,5-dimethylisoxazol-4-yl)vinyl)benzofuran-5-yl)-N-((2,4-dimethylphenyl)(phenyl)methyl)acetamide

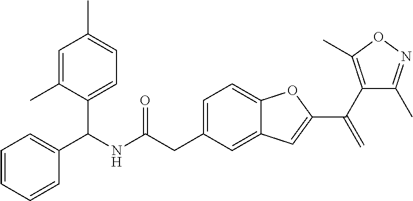 [SMILES: C=C(c1cc2cc(CC(=O)NC(c3ccccc3)c3ccc(C)cc3C)ccc2o1)c1c(C)noc1C]

TFA (23 mg) and Et₃SiH (23 mg) were added to a solution of 2-(2-(1-(3,5-dimethylisoxazol-4-yl)-1-hydroxyethyl)benzofuran-5-yl)-N-((2,4-dimethylphenyl)(phenyl)methyl)acetamide (20 mg, 0.04 mmol) in CH₂Cl₂ (10 mL) at rt, and the reaction mixture was stirred for 3 h. The reaction mixture was then quenched by the addition of water (5 mL), and the mixture was extracted with CH₂Cl₂. The combined extracts were washed with water, dried over sodium sulfate, and concentrated under reduced pressure to obtain 2-(2-(1-(3,5-dimethylisoxazol-4-yOvinyl)benzofuran-5-yl)-N-((2,4-dimethylphenyl)(phenyl)methyl)acetamide (15 mg, yield: 76.5%), which was carried through without further purification. LC-MS: 491.2 [M+H]⁺; Rt: 1.893 min.

### b) 2-(2-(1-(3,5-dimethylisoxazol-4-yl)ethyl)benzofuran-5-yl)-N-((2,4-dimethylphenyl)(phenyl)methyl)acetamide

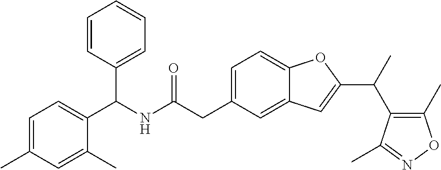

A mixture of 2-(2-(1-(3,5-dimethylisoxazol-4-yl)vinyl)benzofuran-5-yl)-N-((2,4-dimethylphenyl)(phenyl)methyl)acetamide (15 mg) and Pd/C (10 mg) in ethanol (10 mL) was stirred under hydrogen atmosphere at rt for 3 h. The mixture was then filtered through a pad of Celite. The organic layer was concentrated under reduced pressure and the resulting residue which was purified by Pre-HPLC to obtain 2-(2-(1-(3,5-dimethylisoxazol-4-yOethyl)benzofuran-5-yl)-N-((2,4-dimethylphenyl)(phenyl)methyl)acetamide (2 mg, yield: 13%). LC-MS: 493.2 [M+H]⁺; Rt: 1.857 min. ¹H NMR (DMSO, 400 MHz): δ 8.89 (d, J=8.0 Hz, 1H), 7.32-7.13 (m, 7H), 6.99-6.95 (m, 4H), 6.69 (s, 1H), 6.17 (d, J=8.4 Hz, 1H), 4.29-4.24 (m, 1H), 3.57 (s, 2H), 2.33 (s, 3H), 2.26 (s, 3H), 2.16 (s, 3H), 2.14 (s, 3H), 1.57 (d, J=7.2 Hz, 3H).

### Example 59

### 2-(2-((3,5-dimethylisoxazol-4-yl)difluoromethyl)benzofuran-5-yl)-N-((2,4-dimethylphenyl)(phenyl)methyl)acetamide

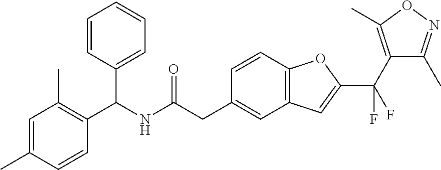

### a) 2-(2-(3,5-dimethylisoxazole-4-carbonyl)benzofuran-5-yl)-N-((2,4-dimethylphenyl)(phenyl)methyl)acetamide

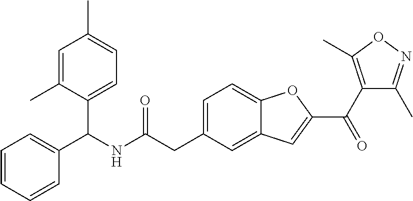 [SMILES: Cc1ccc(C(NC(=O)Cc2ccc3oc(C(=O)c4c(C)noc4C)cc3c2)c2ccccc2)c(C)c1]

MnO₂ (391 mg, 4.5 mmol) was added to a solution of 2-(2-((3,5-dimethylisoxazol-4-yl)(hydroxy)methyl)benzofuran-5-yl)-N-((2,4-dimethylphenyl)(phenyl)methyl)acetamide (741 mg, 1.5 mmol) in CH₂Cl₂ (10 mL) at rt. The reaction mixture was heated to reflux overnight. then cooled to rt and filtered through a pad of Celite. The organic layer was concentrated under reduced pressure to give 2-(2-(3,5-dimethylisoxazole-4-carbonyl)benzofuran-5-yl)-N-((2,4-dimethylphenyl)(phenyl)methyl)acetamide (650 mg, 88%). LC-MS: 493 [M+H]⁺; Rt: 1.810 min.

### b) 2-(2-((3,5-dimethylisoxazol-4-yOdifluoromethyl)benzofuran-5-yl)-N-((2,4-dimethylphenyl)(phenyl)methyl)acetamide

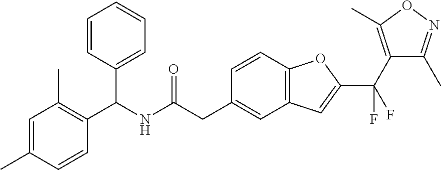

The mixture of 2-(2-(3,5-dimethylisoxazole-4-carbonyl)benzofuran-5-yl)-N-((2,4-dimethylphenyl)(phenyl)methyl)acetamide (20 mg, 0.041 mmol) and DAST (33.8 mg, 0.21 mmol) was heated to 100° C. for 3 h. The mixture was then poured into water, and extracted with ethyl acetate. The combined extracts were washed with brine, dried over sodium sulfate, and concentrated under reduced pressure. The resulting residue was purified by Pre-HPLC to give 2-(24(3,5-dimethylisoxazol-4-yOdifluoromethyl)benzofuran-5-yl)-N-((2,4-dimethylphenyl)(phenyl)methyl)acetamide (13 mg, 62%). LC-MS: 515 [M+H]⁺; Rt: 1.872 min. ¹H NMR (CDCl₃, 500 MHz): δ7.59 (s, 1H), 7.50-7.36 (m, 3H), 7.21-6.73 (m, 7H), 6.31 (d, J=8.0 Hz, 1H), 5.91 (d, J=8.0 Hz, 1H), 3.66 (s, 2H), 2.47 (s, 3H), 2.33 (s, 3H), 2.21 (s, 3H), 2.15 (s, 3H).

### Example 60

### 4-(5-(2-(((2,4-dimethylphenyl)(phenyl)methyl)amino)-2-oxoethyl)benzofuran-2-yl)-5-methylisoxazole-3-carboxylic acid

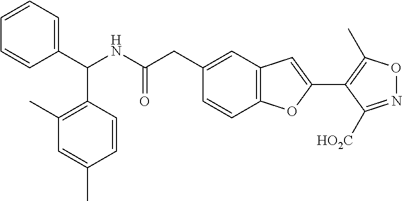 [SMILES: Cc1ccc(C(NC(=O)Cc2ccc3oc(-c4c(C(=O)O)noc4C)cc3c2)c2ccccc2)c(C)c1]

The title compound was synthesized from methyl 4-(5-(2-(((2,4-dimethylphenyl)(phenyl)methyl)amino)-2-oxoethyl)benzofuran-2-yl)-5-methylisoxazole-3-carboxylate following essentially the procedure of Example 31 (b) (6 mg, yield: 36%). LCMS026: 495.2 [M+H]⁺; Rt=1.733 min; Purity=100% (214 nm). ¹H NMR (MeOD, 400 MHz): δ 7.52 (s, 1H), 7.43 (d, J=8.4 Hz, 1H), 7.29 (t, J=7 Hz, 2H), 7.25-7.24 (m, 3H), 7.15 (d, J=7.2 Hz, 2H), 6.98 (s, 1H), 6.95 (d, J=4.0 Hz, 2H), 6.29 (s, 1H), 3.67 (s, 2H), 2.74 (s, 3H), 2.27 (s, 3H), 2.17 (s, 3H).

### Example 61 and 62

### N-((2,4-dimethylphenyl)(phenyl)methyl)-2-(3-(2-methylpyridin-3-yl)quinoxalin-6-yl)acetamide and N-((2,4-dimethylphenyl)(phenyl)methyl)-2-(2-(2-methylpyridin-3-yl)quinoxalin-6-yl)acetamide

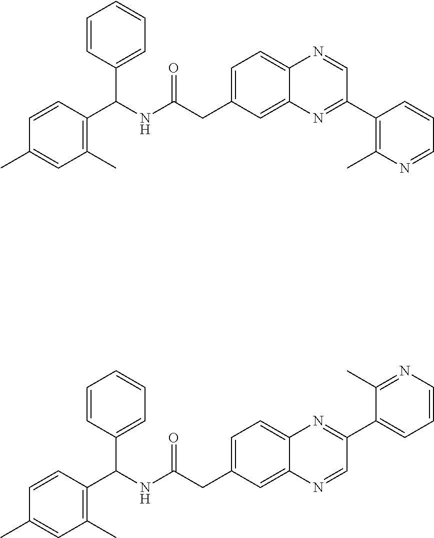 [SMILES: Cc1ccc(C(NC(=O)Cc2ccc3ncc(-c4cccnc4C)nc3c2)c2ccccc2)c(C)c1 ; Cc1ccc(C(NC(=O)Cc2ccc3nc(-c4cccnc4C)cnc3c2)c2ccccc2)c(C)c1]

### a) ethyl 2-(4-acetamido-3-nitrophenyl)acetate

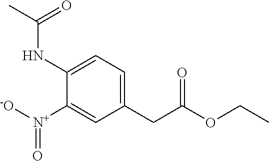

The title compound was synthesized from ethyl 2-(4-aminophenyl)acetate essentially as described in Example 23 (a) (2.1 g, yield: 80%). LC-MS (026): 266.1.0 [M+H]⁺; Rt: 1.777 min, Purity: 53.7% (214 nm).

### b) ethyl 2-(4-amino-3-nitrophenyl)acetate

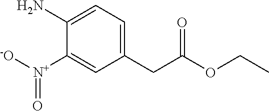

The solution of ethyl 2-(4-acetamido-3-nitrophenyl)acetate (2.1 g, 7.9 mmol) in EtOH (20 mL) and conc. HCl (4 mL) was refluxed overnight. The reaction mixture was then cooled to rt, and concentrated under reduced pressure to ethyl 2-(4-amino-3-nitrophenyl)acetate (1.8 g, yield: 89%). LC-MS (011): 225.10 [M+H]⁺; Rt: 1.816 min, Purity: 88% (254 nm).

### c) ethyl 2-(3,4-diaminophenyl)acetate

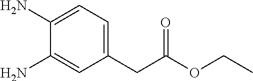

The title compound was synthesized from ethyl 2-(4-amino-3-nitrophenyl)acetate essentially as described in Example 23 (b) (2.2 g, yield: 84.6%). LC-MS (011): 195.10 [M+H]⁺; Rt=1.574 min, purity 83%.

### d) ethyl 2-(3-oxo-3,4-dihydroquinoxalin-6-yl)acetate and ethyl 2-(2-oxo-1,2-dihydroquinoxalin-6-yl)acetate

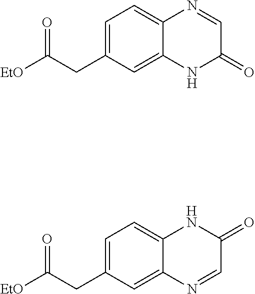 [SMILES: CCOC(=O)Cc1ccc2ncc(=O)[nH]c2c1 ; CCOC(=O)Cc1ccc2[nH]c(=O)cnc2c1]

2-Oxoacetic acid (115 mg, 1.55 mmol) was added to a solution of ethyl 2-(3,4-diaminophenyl)acetate (300 mg, 1.55 mmol) in MeOH (15 mL). The resulting mixture was stirred at rt overnight, then poured into 10 mL of water. The mixture was extracted with EtOAc (10 mL×3). The combined extracts were washed with brine (10 mL), dried over Na₂SO₄, and concentrated under reduced pressure to yield a mixture of ethyl 2-(3-oxo-3,4-dihydroquinoxalin-6-yl)acetate and ethyl 2-(2-oxo-1,2-dihydroquinoxalin-6-yl)acetate (305 mg, yield: 85%). LC-MS (011): 233.10 [M+H]⁺; Rt: 1.379 min, purity 86.7%.

### e) ethyl 2-(3-chloroquinoxalin-6-yl)acetate and ethyl 2-(2-chloroquinoxalin-6-yl)acetate

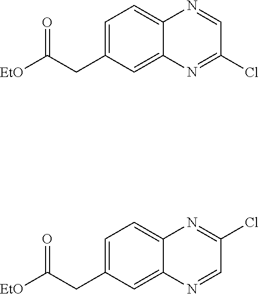 [SMILES: CCOC(=O)Cc1ccc2ncc(Cl)nc2c1 ; CCOC(=O)Cc1ccc2nc(Cl)cnc2c1]

POCl₃ (302.3 mg, 2 mmol) was added to ethyl 2-(3-oxo-3,4-dihydroquinoxalin-6-yl)acetate and ethyl 2-(2-oxo-1,2-dihydroquinoxalin-6-yl)acetate (305 mg, 1.32 mmol), followed by addition of DMF (19.2 mg, 0.263 mmol), then the mixture was refluxed overnight. The reaction mixture was then poured into ice-water, and extracted with EtOAc (15 mL×3). The combined extracts were dried over Na₂SO₄, and concentrated under reduced pressure. The resulting residue was purified by Preh-HPLC to get a mixture of ethyl 2-(3-chloroquinoxalin-6-yl)acetate and ethyl 2-(2-chloroquinoxalin-6-yl)acetate (120 mg, yield: 36%). LC-MS (010): 251.10 [M+H]⁺; Rt=2.058 min.

### f) 2-(3-chloroquinoxalin-6-yl)acetic acid and 2-(2-chloroquinoxalin-6-yl)acetic acid

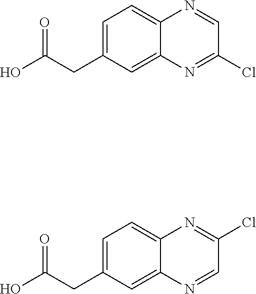 [SMILES: O=C(O)Cc1ccc2ncc(Cl)nc2c1 ; O=C(O)Cc1ccc2nc(Cl)cnc2c1]

The title compounds were synthesized from ethyl 2-(3-chloroquinoxalin-6-yl)acetate and ethyl 2-(2-chloroquinoxalin-6-yl)acetate essentially as described in example 31 (b) (180 mg, yield 82.5%). LC-MS (010): 223.10 [M+H]⁺; Rt=1.87 min, purity 87%.

### g) 2-(3-chloroquinoxalin-6-yl)-N-((2,4-dimethylphenyl)(phenyl)methyl)acetamide and 2-(2-chloroquinoxalin-6-yl)-N4(2,4-dimethylphenyl)(phenyl)methyl)acetamide

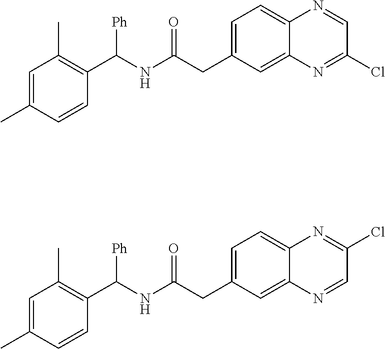 [SMILES: Cc1ccc(C(NC(=O)Cc2ccc3ncc(Cl)nc3c2)c2ccccc2)c(C)c1 ; Cc1ccc(C(NC(=O)Cc2ccc3nc(Cl)cnc3c2)c2ccccc2)c(C)c1]

The title compounds were synthesized from 2-(3-chloroquinoxalin-6-yl)acetic acid and 2-(2-chloroquinoxalin-6-yl)acetic acid and (2,4-dimethylphenyl)(phenyl)methanamine essentially as described in example 2 (b) (200 mg, yield: 30%). LC-MS (024): 416.30 [M+H]⁺; Rt: 1.857 min.

### h) N-((2,4-dimethylphenyl)(phenyl)methyl)-2-(3-(2-methylpyridin-3-yl)quinoxalin-6-yl)acetamide and N-((2,4-dimethylphenyl)(phenyl)methyl)-2-(2-(2-methylpyridin-3-yl)quinoxalin-6-yl)acetamide

The title compounds were synthesized from 2-(3-chloroquinoxalin-6-yl)-N-((2,4-dimethylphenyl)(phenyl)methyl)acetamide and 2-(2-chloroquinoxalin-6-yl)-N-((2,4-dimethylphenyl)(phenyl)methyl)acetamide and (2-methylpyridin-3-yOboronic acid essentially as described in example 11(c):

N-((2,4-dimethylphenyl)(phenyl)methyl)-2-(3-(2-methylpyridin-3-yl)quinoxalin-6-yl)acetamide: LCMS (024): 473.10 [M+H]⁺; Rt=1.422 min. ¹H NMR (DMSO-d₆, 400 MHz): δ 9.00 (s, 1H), 8.65 (d, J=4 Hz, 1H), 8.13 (t, J=8.4 Hz, 1H), 8.04 (s, 1H), 7.90 (t, J=6 Hz, 1H), 7.79 (t, J=5.6 Hz, 1H), 7.35-7.32 (m, 1H), 7.29-7.22 (m, 3H), 7.12 (d, J=7.2 Hz, 2H), 6.98 (s, 1H), 6.92 (d, J=8 Hz, 1H), 6.85 (d, J=7.6 Hz, 1H), 6.40 (d, J=8 Hz, 1H), 6.13 (d, J=8.4 Hz, 1H), 3.88 (s, 2H), 2.71 (s, 3H), 2.28 (s, 3H), 2.24 (s, 3H).

N-((2,4-dimethylphenyl)(phenyl)methyl)-2-(2-(2-methylpyridin-3-yl)quinoxalin-6-yl)acetamide: LCMS (024): 473.10 [M+H]⁺; Rt=1.422 min. 1H NMR (DMSO-d6, 400 MHz): δ 9.00 (s, 1H), 8.65 (d, J=4 Hz, 1H), 8.14 (d, J=8.4 Hz, 1H), 8.04 (s, 1H), 7.88 (d, J=8 Hz, 1H), 7.78 (d, J=8.8 Hz, 1H), 7.35-7.32 (m, 1H), 7.29-7.22 (m, 3H), 7.11 (d, J=6.8 Hz, 2H), 6.98 (s, 1H), 6.92 (d, J=8 Hz, 1H), 6.85 (d, J=7.6 Hz, 1H), 6.40 (d, J=8 Hz, 1H), 6.13 (d, J=8.4 Hz, 1H), 3.88 (s, 2H), 2.71 (s, 3H), 2.27 (s, 3H), 2.23 (s, 3H).

### Example 63

### 2-(2-((3,5-dimethylisoxazol-4-yl)(2-methoxy-2-methylpropoxy)methyl)benzofuran-5-yl)-N-((2,4-dimethylphenyl)(phenyl)methyl)acetamide

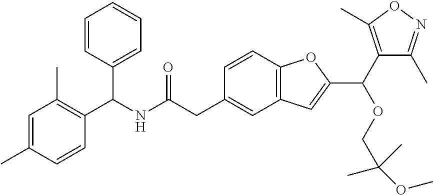

### a) 2-methoxy-2-methylpropan-1-ol

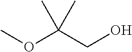

A solution of 2,2-dimethyloxirane (32 g, 0.44 mol) in MeOH (65 mL) was added drop-wise to a solution of concentrated H₂SO₄ (9.8 mL) in MeOH (5 mL). The solution was heated under reflux for 4 h, and then cooled to rt. The pH value of the mixture was adjusted to 7 with a KOH solution in MeOH. The solvent was concentrated under reduced pressure at rt to give yellow oil (60 mL). The oil was distilled at atmospheric pressure to yield 2-methoxy-2-methylpropan-1-ol as colorless liquid (b.p: 137° C., 20 g, yield 43%.)

### b) 2-(2-((3,5-dimethylisoxazol-4-yl)(2-methoxy-2-methylpropoxy)methyl)benzofuran-5-yl)-N-((2,4-dimethylphenyl)(phenyl)methyl)acetamide

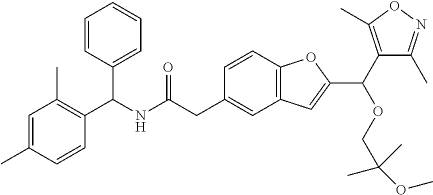

2-Methoxy-2-methylpropan-1-ol (12 mg, 0.12 mmol) and CF₃COOH (41 mg, 0.36 mmol) were added to a solution of compound 2-(2-((3,5-dimethylisoxazol-4-yl)(hydroxy)methyl)benzofuran-5-yl)-N-((2,4-dimethylphenyl)(phenyl)methyl)acetamide (30 mg, 0.06 mmol) in CH₂Cl₂ (10 mL) under N₂. The mixture was stirred at rt for 12 h., and then extracted with ethyl acetate (30 mL×3). The combined extracts were washed with water (30 mL) and brine (15 mL×3), dried over Na₂SO₄, and concentrated under reduced pressure to to give yellow oil. The oil was purified by Pre-TLC using 30% EtOAc in petroleum ether to give 2424(3,5-dimethylisoxazol-4-yl)(2-methoxy-2-methylpropoxy)methyl)benzofuran-5-yl)-N-((2,4-dimethylphenyl)(phenyl)methyl)acetamide as awhite solid (5 mg, yield 14%). LC-MS A024: fragment at 477, Rt=1.93 min. ¹H NMR (MeOD, 400 MHz): δ 7.52 (s, 1H), 7.39 (d, J=8.4 Hz, 1H), 7.28-7.23 (m, 4H), 7.14 (d, J=6.8 Hz, 2H), 6.98 (s, 1H), 6.93-6.92 (m, 2H), 6.76 (s, 1H), 6.28 (s, 1H), 5.63 (s, 1H), 3.67 (s, 2H), 3.48 (d, J=10 Hz, 1H), 3.44 (d, J=10 Hz, 1H), 3.23 (s, 3H), 2.44 (s, 3H), 2.27 (s, 3H), 2.24 (s, 3H), 2.16 (s, 3H), 1.21 (s, 6H).

### Examples 64 and 65

### 2-(2-(((1H-tetrazol-5-yl)methoxy)(3,5-dimethylisoxazol-4-yl)methyl)benzofuran-5-yl)-N-((2,4-dimethylphenyl)(phenyl)methyl)acetamide and 2-(2-((3,5-dimethylisoxazol-4-yl)(5-(hydroxymethyl)-1H-tetrazol-1-yl)methyl)benzofuran-5-yl)-N-((2,4-dimethylphenyl)(phenyl)methyl)acetamide

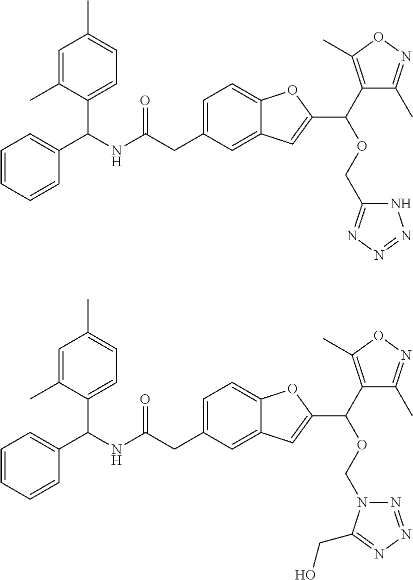 [SMILES: Cc1ccc(C(NC(=O)Cc2ccc3oc(C(OCc4nnn[nH]4)c4c(C)noc4C)cc3c2)c2ccccc2)c(C)c1 ; Cc1ccc(C(NC(=O)Cc2ccc3oc(C(OCn4nnnc4CO)c4c(C)noc4C)cc3c2)c2ccccc2)c(C)c1]

The title compounds were synthesized from 2-(2-((3,5-dimethylisoxazol-4-yl)(hydroxy)methyl)benzofuran-5-yl)-N-((2,4-dimethylphenyl)(phenyl)methyl)acetamide and (1H-tetrazol-5-yl)methanol following essentially the procedure of Example 63 (b):

2-(2-(((1H-tetrazol-5-yl)methoxy)(3,5-dimethylisoxazol-4-yl)methyl)benzofuran-5-yl)-N-((2,4-dimethylphenyl)(phenyl)methyl)acetamide: 4 mg (yield: 3.5%); LC-MS012: fragment at 477.1 [M−99]⁺; Rₜ=1.645 min; ¹H NMR (DMSO, 400 MHz): δ 8.94 (d, J=8.0 Hz, 1H), 7.58-7.55 (m, 3H), 7.36-7.21 (m, 6H), 7.05-6.98 (m, 3H), 6.74 (s, 1H), 6.23 (d, J=8.4 Hz, 1H), 6.16 (t, J=5.8 Hz, 1H), 5.05-4.94 (m, 2H), 3.66 (s, 2H), 2.41 (s, 3H), 2.29 (s, 3H), 2.20 (s, 3H), 2.19 (s, 3H).

2-(2-((3,5-dimethylisoxazol-4-yl)(5-(hydroxymethyl)-1H-tetrazol-1-yl)methyl)benzofuran-5-yl)-N-((2,4-dimethylphenyl)(phenyl)methyl)acetamide: 23 mg (yield: 20%); LC-MS024: fragment at 477.2 [M−99]⁺; Rt=1.724 min; ¹H NMR (DMSO, 400 MHz): δ 8.87 (d, J=8.4 Hz, 1H), 7.90 (s, 1H), 7.52-7.14 (m, 8H), 6.98-6.92 (m, 3H), 6.82 (s, 1H), 6.17 (d, J=8.4 Hz, 1H), 5.64 (t, J=6.0 Hz, 1H), 4.70 (d, J=6.0 Hz, 2H), 3.60 (s, 2H), 2.35 (s, 3H), 2.22 (s, 3H), 2.13 (s, 3H), 2.09 (s, 3H).

### Examples 66 and 67

### methyl 2-((3,5-dimethylisoxazol-4-yl)(5-(2-(((2,4-dimethylphenyl)(phenyl)methyl)amino)-2-oxoethyl)benzofuran-2-yl)methoxy)-2-methylpropanoate and 2-(2-((3,5-dimethylisoxazol-4-yl)(methoxy)methyl)benzofuran-5-yl)-N-((2,4-dimethylphenyl)(phenyl)methyl)acetamide

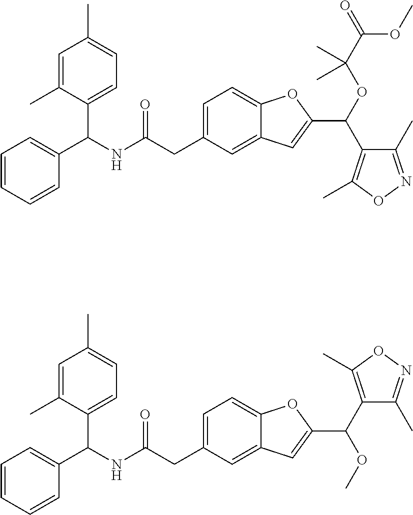 [SMILES: COC(=O)C(C)(C)OC(c1cc2cc(CC(=O)NC(c3ccccc3)c3ccc(C)cc3C)ccc2o1)c1c(C)noc1C ; COC(c1cc2cc(CC(=O)NC(c3ccccc3)c3ccc(C)cc3C)ccc2o1)c1c(C)noc1C]

The title compounds were synthesized from 2-(2-((3,5-dimethylisoxazol-4-yl)(hydroxy)methyl)benzofuran-5-yl)-N-((2,4-dimethylphenyl)(phenyl)methyl)acetamide and methyl 2-hydroxy-2-methylpropanoate following essentially the procedure of Example 63 (b):

methyl 2-((3,5-dimethylisoxazol-4-yl)(5-(2-((2,4-dimethylphenyl)(phenyl)methyl)amino)-2-oxoethyl)benzofuran-2-yOmethoxy)-2-methylpropanoate: 3 mg (yield: 12%); LC-MS: 595.3 [M+H]⁺; Rt: 1.906 min; ¹H NMR (CDCl₃, 400 MHz): δ 7.33-7.30 (m, 2H), 7.19-7.08 (m, 4H), 6.97-6.80 (m, 4H), 6.65 (d, J=7.6 Hz, 1H), 6.46 (s, 1H), 6.29 (d, J=8.4 Hz, 1H), 5.84 (d, J=7.6 Hz, 1H), 5.60 (s, 1H), 3.63 (s, 2H), 3.45 (s, 3H), 2.37 (s, 3H), 2.20 (s, 6H), 2.12 (s, 3H), 1.48 (s, 3H), 1.41 (s, 3H).

2-(2-((3,5-dimethylisoxazol-4-yl)(methoxy)methyl)benzofuran-5-yl)-N-((2,4-dimethylphenyl)(phenyl)methyl)acetamide: 2 mg (yield: 7%) LC-MS: 509.2 [M+H]⁺; Rt=1.856 min; ¹H NMR (CDCl₃, 400 MHz): δ 7.36-7.34 (m, 2H), 7.19-7.10 (m, 4H), 6.98-6.80 (m, 4H), 6.66 (d, J=7.6 Hz, 1H), 6.49 (s, 1H), 6.29 (d, J=8.0 Hz, 1H), 5.85 (d, J=8.0 Hz, 1H), 5.24 (s, 1H), 3.63 (s, 2H), 3.36 (s, 3H), 2.35 (s, 3H), 2.20 (s, 3H), 2.18 (s, 3H), 2.12 (s, 3H).

### Example 68

### 2-(2-(((2-amino-2-oxoethyl)amino)(3,5-dimethylisoxazol-4-yl)methyl)benzofuran-5-yl)-N-((2,4-dimethylphenyl)(phenyl)methyl)acetamide

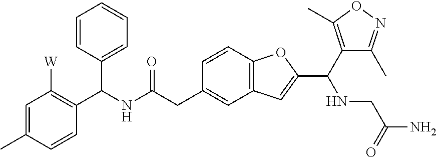

The title compounds were synthesized from 2-(2-(chloro(3,5-dimethylisoxazol-4-yl)methyl)benzofuran-5-yl)-N-((2,4-dimethylphenyl)(phenyl)methyl)acetamide and 2-aminoacetamide hydrochloride following essentially the procedure of Example 37 (a) (10 mg, yield 23%). LC-MSA026: no mass [M+H]⁺, fragment observed at 477; Rt=1.92 min. ¹H NMR (CDCl₃, 400 MHz): δ 7.43 (s, 1H), 7.42 (d, J=8.8 Hz, 1H), 7.39-7.17 (m, 4H), 7.06 (d, J=7.2 Hz, 2H), 6.98 (s, 1H), 6.91 (d, J=8.0 Hz, 1H), 6.75 (d, J=7.6 Hz, 1H), 6.55 (s, 1H), 6.50 (s, 1H), 6.38 (d, J=8.0 Hz, 1H), 5.93 (d, J=8.0 Hz, 1H), 5.52 (s, 1H), 4.95 (s, 1H), 3.71 (s, 2H), 3.38 (s, 2H), 2.43 (s, 3H), 2.28 (s, 3H), 2.27 (s, 3H), 2.22 (s, 3H), 2.10 (s, 1H).

### Example 69

### 2-(2-(3,5-dimethylisoxazol-4-yl)benzofuran-5-yl)-N-(4-methyl-1-(p-tolyl)pentyl)acetamide

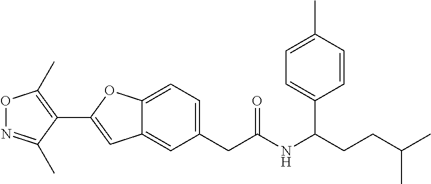 [SMILES: Cc1ccc(C(CCC(C)C)NC(=O)Cc2ccc3oc(-c4c(C)noc4C)cc3c2)cc1]

### a) 4-methyl-1-(p-tolyl)pentan-1-amine

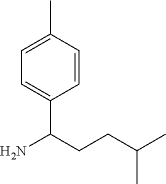

A catalytic amount of I₂ and around 0.2 gram of 1-bromo-3-methylbutane were added to a suspension of Mg (1.87 g, 77.86 mmol) in THF (200 mL). The reaction was initiated by heating, and the remained 1-bromo-3-methylbutane (9.8 g, 64.8 mmol) was added drop-wise. The mixture was stirred at rt for 4 h under nitrogen. 4-Methylbenzonitrile (5 g, 41.83 mmol) was added dropwise into the solution. After the addition, the reaction mixture was refluxed for 5 h. The reaction mixture was then cooled to rt and quenched by addition 20 mL of MeOH. NaBH₄ (4.9 g, 125.5 mmol) and MeOH (100 mL) were then added to the reaction mixture. After stirring at rt overnight, the reaction was quenched by the addition of water (100 mL). Solvent was removed under reduced pressure. The residue was extracted with CH₂Cl₂ (100 mL×3). The combined extracts were concentrated under reduced pressure and the resulting residue was purified by silica gel column chromatography (petroleum ether/EtOAc=1:1) to give 4-methyl-1-(p-tolyl)pentan-1-amine as a yellow solid (500 mg, Yield: 6.26%). LC-MS: MS=176 [M+1]⁺; Rt=1.26 min.

### b) 2-(2-(3,5-dimethylisoxazol-4-yl)benzofuran-5-yl)-N-(4-methyl-1-(p-tolyl-pentyl)acetamide

The title compounds were synthesized from 2-(2-(3,5-dimethylisoxazol-4-yl)benzofuran-5-yl)acetic acid and 4-methyl-1-(p-tolyl)pentan-1-amine following essentially the procedure of Example 2 (b) (30 mg, Yield: 46.9%). LC-MS022: m/z 445.7 [M+H]⁺; Rt=1.986 min. ¹H NMR (DMSO, 400 MHz): δ 8.42 (d, J=5.2 Hz, 1H), 7.52 (d, J=7.6 Hz, 2H), 7.22 (dd, J₁=1.2 Hz, J₂=6.8 Hz, 1H), 7.16 (d, J=6 Hz, 2H), 7.09 (d, J=6.4 Hz, 2H), 7.05 (s, 1H), 4.70-4.65 (m, 1H), 3.54-3.33 (m, 2H), 2.66 (s, 3H), 2.44 (s, 3H), 2.25 (s, 1H), 1.63-1.60 (m, 2H), 1.49-1.44 (m, 1H), 1.13-1.02 (m, 2H), 0.81-0.77 (m, 6H).

### Examples 70 and 71

### 2-(1-(2-amino-2-oxoethyl)-2-(3,5-dimethylisoxazol-4-yl)-1H-benzo[d]imidazol-6-yl)-N-((2,4-dimethylphenyl)(phenyl)methyl)acetamide and 2-(1-(2-amino-2-oxoethyl)-2-(3,5-dimethylisoxazol-4-yl)-1H-benzo[d]imidazol-5-yl)-N-((2,4-dimethylphenyl)(phenyl)methyl)acetamide

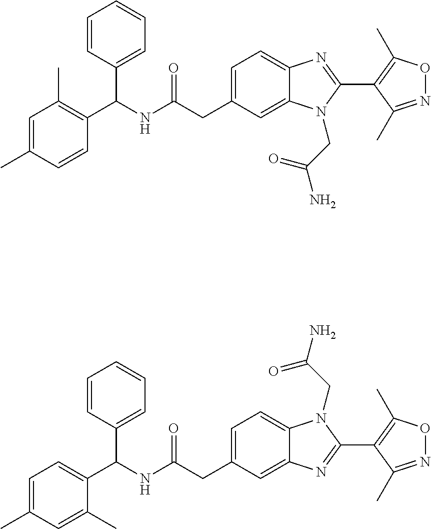 [SMILES: Cc1ccc(C(NC(=O)Cc2ccc3nc(-c4c(C)noc4C)n(CC(N)=O)c3c2)c2ccccc2)c(C)c1 ; Cc1ccc(C(NC(=O)Cc2ccc3c(c2)nc(-c2c(C)noc2C)n3CC(N)=O)c2ccccc2)c(C)c1]

### a) ethyl 2-(4-(3,5-dimethylisoxazole-4-carboxamido)phenyl)acetate

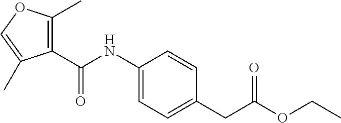 [SMILES: CCOC(=O)Cc1ccc(NC(=O)c2c(C)coc2C)cc1]

A mixture of ethyl 2-(4-aminophenyl)acetate (6.35 g, 0.0355 mol), EDCI (13.6 g, 0.071 mol) and HOBt (4.8 g, 0.0355 mmol) in DCM (100 mL) was stirred at rt for 0.5 hr. Then 3,5-dimethylisoxazole-4-carboxylic acid (5 g, 0.0355 mol) was added, after the addition the mixture was stirred at rt for overnight. The reaction mixture was then washed with water (20 mL×3) and saturated solution of sodium hydrogen carbonate (20 mL×3), dried over anhydrous sodium sulfate, and concentrated under reduced pressure. The resulting residue was purified by column chromatography (petroleum ether/EtOAc:5/1˜2/1) to obtain white solid ethyl 2-(4-(3,5-dimethylisoxazole-4-carboxamido)phenyl)acetate (6.6 g, yield: 62%). LC-MS (022): 303.0 [M+H]⁺; Rt=1.58 min. purity 100% (214 nm).

### b) ethyl 2-(4-(3,5-dimethylisoxazole-4-carboxamido)-3-nitrophenyl)acetate

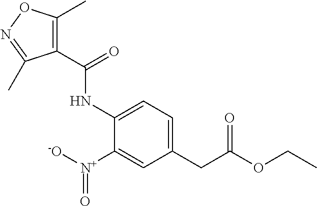

Fuming nitric acid (0.82 g, 0.013 mol) was added to a cooled (−10° C.) solution of ethyl 2-(4-(3,5-dimethylisoxazole-4-carboxamido)phenyl)acetate (3.3 g, 0.011 mol) in trifluoroacetic acid anhydride (30 mL). After the addition, the reaction mixture was stirred at −10° C. for 1 h. The reaction mixture was then poured over ice-water (200 mL) forming yellow a precipitate. The precipitate was collected by filtration and dried under reduced pressure to give ethyl 2-(4-(3,5-dimethylisoxazole-4-carboxamido)-3-nitrophenyl)acetate (3 g, yield: 78.6%). LC-MS (010): 348.0 [M+H]⁺; Rt=2.19 min. purity: 98% (214 nm)

### c) ethyl 2-(2-(3,5-dimethylisoxazol-4-yl)-1H-benzo[d]imidazol-6-yl)acetate

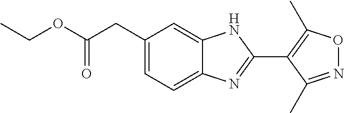 [SMILES: CCOC(=O)Cc1ccc2nc(-c3c(C)noc3C)[nH]c2c1]

Zinc (121.7 mg, 1.87 mmol) was added to a solution of ethyl 2-(4-(3,5-dimethylisoxazole-4-carboxamido)-3-nitrophenyl)acetate (130 mg, 0.375 mmol) in acetic acid (5 mL). The reaction mixture was then stirred at 80° C. for 2 h, filtered and concentrated under reduced pressure. The residue was dissolved in ethyl acetate (30 mL), washed with saturated solution of sodium hydrogen carbonate (5 mL×3), dried over anhydrous sodium sulfate, and concentrated under reduced pressure to yield ethyl 2-(2-(3,5-dimethylisoxazol-4-yl)-1H-benzo[d]imidazol-6-yl)acetate (151 mg), which was used for next step without any purification. LC-MS (010): 300.0 [M+H]⁺; Rt=2.00 min.

### d) tert-butyl 2-(2-(3,5-dimethylisoxazol-4-yl)-6-(2-ethoxy-2-oxoethyl)-1H-benzo[d]imidazol-1-yl)acetate and ethyl 2-(2-(3,5-dimethylisoxazol-4-yl)-1-(2-isoprop oxy-2-oxoethyl)-1H-benzo[d]imidazol-5-yl)acetate

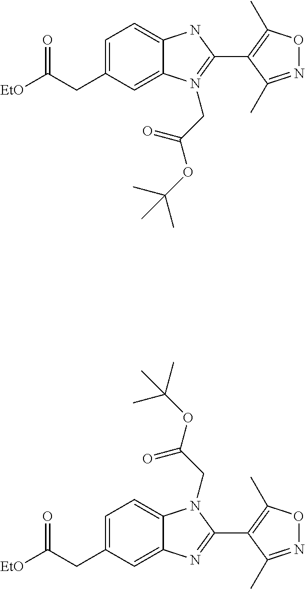

A mixture of ethyl 2-(2-(3,5-dimethylisoxazol-4-yl)-1H-benzo[d]imidazol-6-yl)acetate (1.058 g, 3.54 mmol), tert-butyl 2-bromoacetate (897 mg, 4.60 mmol) and potassium carbonate (977 mg, 7.08 mmol) in tetrahydrofuran (10 mL) was stirred at 80° C. overnight. The reaction mixture was then filtered, and the filtrate was concentrated under reduced pressure. The resulted residue was purified by column chromatography (petroleum ether/EtOAc:3/11/1) to obtain tert-butyl 2-(2-(3,5-dimethylisoxazol-4-yl)-6-(2-ethoxy-2-oxoethyl)-1H-benzo[d]imidazol-1-yl)acetate and ethyl 2-(2-(3,5-dimethylisoxazol-4-yl)-1-(2-isopropoxy-2-oxoethyl)-1H-benzo[d]imidazol-5-yl)acetate as a mixture (912 mg, yield: 62.3%). LC-MS (022): 414.0 [M+H]⁺; Rt=1.55 min.

### e) 2-(2-(3,5-dimethylisoxazol-4-yl)-6-(2-ethoxy-2-oxoethyl)-1H-benzo[d]imidazol-1-yl)acetic acid and 2-(2-(3,5-dimethylisoxazol-4-yl)-5-(2-ethoxy-2-oxoethyl)-1H-benzo[d]imidazol-1-yl)acetic acid

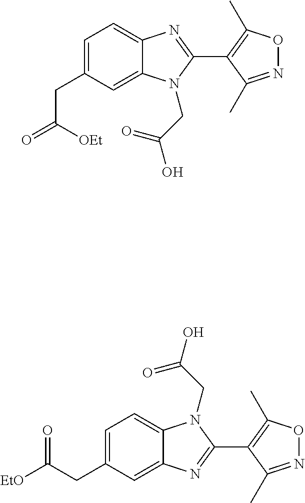 [SMILES: CCOC(=O)Cc1ccc2nc(-c3c(C)noc3C)n(CC(=O)O)c2c1 ; CCOC(=O)Cc1ccc2c(c1)nc(-c1c(C)noc1C)n2CC(=O)O]

The title compounds were synthesized from tert-butyl 2-(2-(3,5-dimethylisoxazol-4-yl)-6-(2-ethoxy-2-oxoethyl)-1H-benzo[d]imidazol-1-yl)acetate and ethyl 2-(2-(3,5-dimethylisoxazol-4-yl)-1-(2-isopropoxy-2-oxoethyl)-1H-benzo[d]imidazol-5-yl)acetate essentially as described in example 39 (f) (420 mg). LC-MS (011): 358.1 [M+H]⁺; Rt=1.30 min. purity: 95% (214 nm)

### f) ethyl 2-(1-(2-amino-2-oxoethyl)-2-(3,5-dimethylisoxazol-4-yl)-1H-benzo[d]imidazol-6-yl)acetate and ethyl 2-(1-(2-amino-2-oxoethyl)-2-(3,5-dimethylisoxazol-4-yl)-1H-benzo[d]imidazol-5-yl)acetate

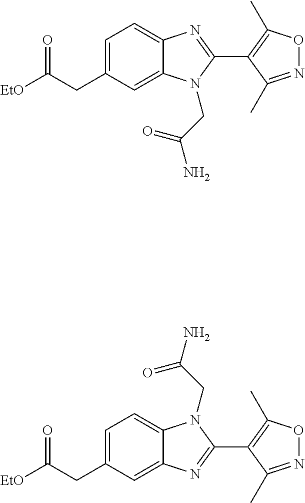 [SMILES: CCOC(=O)Cc1ccc2nc(-c3c(C)noc3C)n(CC(N)=O)c2c1 ; CCOC(=O)Cc1ccc2c(c1)nc(-c1c(C)noc1C)n2CC(N)=O]

The title compounds were synthesized from 2-(2-(3,5-dimethylisoxazol-4-yl)-6-(2-ethoxy-2-oxoethyl)-1H-benzo[d]imidazol-1-yl)acetic acid and 2-(2-(3,5-dimethylisoxazol-4-yl)-5-(2-ethoxy-2-oxoethyl)-1H-benzo[d]imidazol-1-yl)acetic acid essentially as described in example 43, except it was carried onto the next step without purification. LC-MS (022): 329.1 [M+H]⁺; Rt=1.93 min.

### g) 2-(1-(2-amino-2-oxoethyl)-2-(3,5-dimethylisoxazol-4-yl)-1H-benzo[d]imidazol-6-yl)acetic acid and 2-(1-(2-amino-2-oxoethyl)-2-(3,5-dimethylisoxazol-4-yl)-1H-benzo[d]imidazol-5-yl)acetic acid

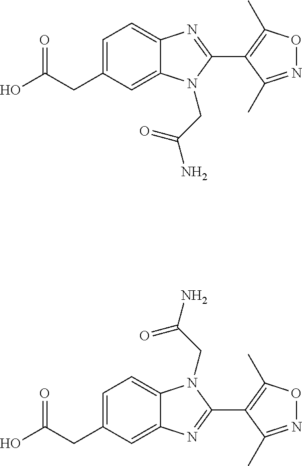 [SMILES: Cc1noc(C)c1-c1nc2ccc(CC(=O)O)cc2n1CC(N)=O ; Cc1noc(C)c1-c1nc2cc(CC(=O)O)ccc2n1CC(N)=O]

The title compounds were synthesized from ethyl 2-(1-(2-amino-2-oxoethyl)-2-(3,5-dimethylisoxazol-4-yl)-1H-benzo[d]imidazol-6-yl)acetate and ethyl 2-(1-(2-amino-2-oxoethyl)-2-(3,5-dimethylisoxazol-4-yl)-1H-benzo[d]imidazol-5-yl)acetate essentially as described in example 1 (f) (230 mg, crude) and they were carried through without further purification

### h) 2-(1-(2-amino-2-oxoethyl)-2-(3,5-dimethylisoxazol-4-yl)-1H-benzo[d]imidazol-6-yl)-N-((2,4-dimethylphenyl)(phenyl)methyl)acetamide and 2-(1-(2-amino-2-oxoethyl)-2-(3,5-dimethylisoxazol-4-yl)-1H-benzo[d]imidazol-5-yl)-N-((2,4-dimethylphenyl)(phenyl)methyl)acetamide

The title compounds were synthesized from 2-(1-(2-amino-2-oxoethyl)-2-(3,5-dimethylisoxazol-4-yl)-1H-benzo[d]imidazol-6-yl)acetic acid and 2-(1-(2-amino-2-oxoethyl)-2-(3,5-dimethylisoxazol-4-yl)-1H-benzo[d]imidazol-5-yl)acetic acid and (2,4-dimethylphenyl)(phenyl)methanamine essentially as described in example 2 (b):

2-(1-(2-amino-2-oxoethyl)-2-(3,5-dimethylisoxazol-4-yl)-1H-benzo[d]imidazol-6-yl)-N-((2,4-dimethylphenyl)(phenyl)methyl)acetamide: (8 mg, yield: 1.8%, 4 steps); LC-MS (022): 522.25 [M+H]⁺; Rt=1.52 min. ¹H NMR (MEOD, 400 MHz): δ 7.70 (s, 1H), 7.46 (d, J=8.4 Hz, 1H), 7.37 (dd, J=8.4 Hz, J=1.2 Hz, 1H), 7.31-7.24 (m, 3H), 7.16 (s, 1H), 7.15 (s, 1H), 6.99-6.95 (m, 3H), 6.31 (s, 1H), 4.83 (s, 2H), 3.75 (s, 2H), 2.42 (s, 3H), 2.28 (s, 3H), 2.23 (s, 3H), 2.18 (s, 3H).

2-(1-(2-amino-2-oxoethyl)-2-(3,5-dimethylisoxazol-4-yl)-1H-benzo[d]imidazol-5-yl)-N-((2,4-dimethylphenyl)(phenyl)methyl)acetamide: (10 mg, yield: 2.1%, 4 steps); LC-MS (022): 522.25 [M+H]⁺; Rt=1.52 min. ¹H NMR (MEOD, 400 MHz): δ 7.66 (d, J=8.4 Hz, 1H), 7.46 (s, 1H), 7.33-7.24 (m, 4H), 7.17 (s, 1H), 7.15 (s, 1H), 7.00-6.96 (m, 3H), 6.32 (s, 1H), 4.80 (s, 2H), 3.76 (s, 2H), 2.42 (s, 3H), 2.28 (s, 3H), 2.23 (s, 3H), 2.19 (s, 3H).

### Examples 72 and 73

### 2-(2-((R)-(2-amino-2-methylpropoxy)(3,5-dimethylisoxazol-4-yl)methyl)benzofuran-5-yl)-N—((S)-(2,4-dimethylphenyl)(phenyl)methyl)acetamide and 2-(2-((S)-(2-amino-2-methylpropoxy)(3,5-dimethylisoxazol-4-yl)methyl)benzofuran-5-yl)-N—((S)-(2,4-dimethylphenyl)(phenyl)methyl)acetamide

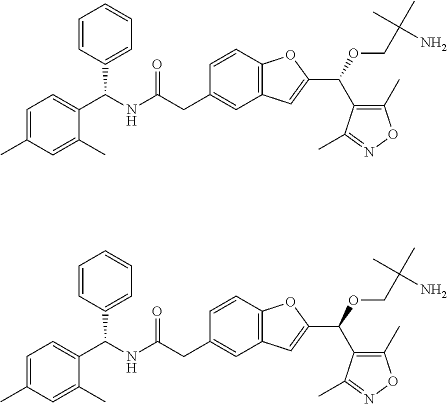 [SMILES: Cc1ccc([C@@H](NC(=O)Cc2ccc3oc([C@H](OCC(C)(C)N)c4c(C)noc4C)cc3c2)c2ccccc2)c(C)c1 ; Cc1ccc([C@@H](NC(=O)Cc2ccc3oc([C@@H](OCC(C)(C)N)c4c(C)noc4C)cc3c2)c2ccccc2)c(C)c1]

### a) 2-(2-((2-amino-2-methylpropoxy)(3,5-dimethylisoxazol-4-yl)methyl)benzofuran-5-yl)-N—((S)-(2,4-dimethylphenyl)(phenyl)methyl)acetamide

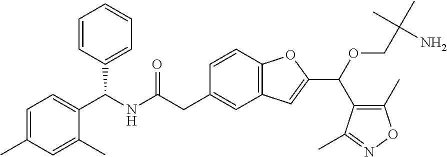 [SMILES: Cc1ccc([C@@H](NC(=O)Cc2ccc3oc(C(OCC(C)(C)N)c4c(C)noc4C)cc3c2)c2ccccc2)c(C)c1]

The title compound was synthesized from 2-(2-((3,5-dimethylisoxazol-4-yl)(hydroxy)methyl)benzofuran-5-yl)acetic acid and (S)-(2,4-dimethylphenyl)(phenyl)methanamine hydrochloride following essentially the procedures of Example 13 (c) and then Example 35 (600 mg, yield 80.4%). LC-MSA024: 566.5 [M+H]⁺, Rt=1.45 min, purity96% (254 nm).

### b) 2-(2-((R)-(2-amino-2-methylpropoxy)(3,5-dimethylisoxazol-4-yl)methyl)benzofuran-5-yl)-N—((S)-(2,4-dimethylphenyl)(phenyl)methyl)acetamide and 2-(2-((S)-(2-amino-2-methylpropoxy)(3,5-dimethylisoxazol-4-yl)methyl)benzofuran-5-yl)-N—((S)-(2,4-dimethylphenyl)(phenyl)methyl)acetamide

2-(2-((2-amino-2-methylpropoxy)(3,5-dimethylisoxazol-4-yl)methyl)benzofuran-5-yl)-N-((S)-(2,4-dimethylphenyl)(phenyl)methyl)acetamide (600 mg) was resolved using the following method:

Instrument: Thar SFC Prep 80 (Thar Technologies, Waters); Column: ChiralPak AD-H, 30 mm I.D.×250 mm Length, 5 μm (Daicel Chemical Industries Co., Ltd); Column Temperature: 35° C.; Mobile Phase: CO₂/MeOH/DEA=75/25/0.1; Flow rate: 80 g/min; Back Pressure: 100 Bar; Wavelength: 214 nm; Cycle time: 9.0 min; Injection Volume: 2.0 mL; Load per injection: 33.3 mg. Said resolution yielded 2 diastereomers:
—peak 1, 103 mg, RT 3.4 min, >99% de; —peak 2, 115 mg, R_{T} 4.8 min, >99% de

Peak 1: LC-MSA024: 566.4 [M+H]⁺; Rt=1.47 min, ¹H NMR (MeOD, 400 MHz): δ 7.52 (s, 1H), 7.39 (d, J=8.0 Hz, 1H), 7.28-7.23 (m, 4H), 7.14 (d, J=6.8 Hz, 2H), 6.98 (s, 1H), 6.94-6.93 (m, 2H), 6.78 (s, 1H), 6.29 (s, 1H), 5.61 (s, 1H), 3.66 (s, 2H), 3.36-3.35 (m, 2H), 2.44 (s, 3H), 2.27 (s, 3H), 2.25 (s, 3H), 2.16 (s, 3H), 1.18 (s, 6H).

Peak 2: LC-MSA024: 566.5[M+H]⁺; Rt=1.49 min. ¹H NMR (MeOD, 400 MHz): δ 7.53 (s, 1H), 7.41 (d, J=8.8 Hz, 1H), 7.28-7.24 (m, 4H), 7.14 (d, J=7.6 Hz, 2H), 6.98 (s, 1H), 6.95-6.94 (m, 2H), 6.82 (s, 1H), 6.29 (s, 1H), 5.71 (s, 1H), 3.67 (s, 2H), 3.56-3.54 (m, 2H), 2.44 (s, 3H), 2.27 (s, 6H), 2.17 (s, 3H), 1.38 (dd, J=8.0 Hz, 3.6 Hz, 6H).

### Example 74

### 2-(2-((2-amino-2-methylpropoxy)(3,5-dimethylisoxazol-4-yl)methyl)benzofuran-5-yl)-N-(1-(2,4-dimethylphenyl)-4-methylpentyl)acetamide

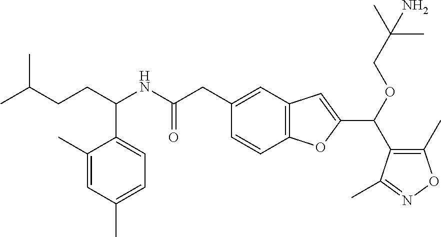 [SMILES: Cc1ccc(C(CCC(C)C)NC(=O)Cc2ccc3oc(C(OCC(C)(C)N)c4c(C)noc4C)cc3c2)c(C)c1]

### a) 2-(2-((3,5-dimethylisoxazol-4-yl)(hydroxy)methyl)benzofuran-5-yl)-N-(1-(2,4-dimethylphenyl)-4-methylpentyl)acetamide

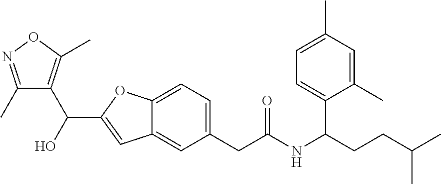 [SMILES: Cc1ccc(C(CCC(C)C)NC(=O)Cc2ccc3oc(C(O)c4c(C)noc4C)cc3c2)c(C)c1]

The title compound was synthesized from 2-(2-((3,5-dimethylisoxazol-4-yl)(hydroxy)methyl)benzofuran-5-yl)acetic acid and 1-(2,4-dimethylphenyl)-4-methylpentan-1-amine following essentially the procedure of Example 2 (b) (251 mg, yield: 62%). LCMSA(022): 489.0 [M+H]⁺; Rt=1.876 min; Purity=75.3% (214 nm).

### b) 2-(2-(chloro(3,5-dimethylisoxazol-4-yl)methyl)benzofuran-5-yl)-N-(1-(2,4-dimethylphenyl)-4-methylpentyl)acetamide

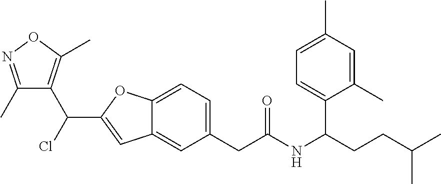 [SMILES: Cc1ccc(C(CCC(C)C)NC(=O)Cc2ccc3oc(C(Cl)c4c(C)noc4C)cc3c2)c(C)c1]

The title compound was synthesized from 2-(2-((3,5-dimethylisoxazol-4-yl)(hydroxy)methyl)benzofuran-5-yl)-N-(1-(2,4-dimethylphenyl)-4-methylpentyl)acetamide following essentially the procedure of Example 28 (a) (143 mg, 100%). LCMSA (022): 503.0 [M-Cl⁺ MeO+H]⁺; Rt: 1.986 min.

### c) 2-(2-((2-amino-2-methylpropoxy)(3,5-dimethylisoxazol-4-yl)methyl)benzofuran-5-yl)-N-(1-(2,4-dimethylphenyl)-4-methylpentyl)acetamide

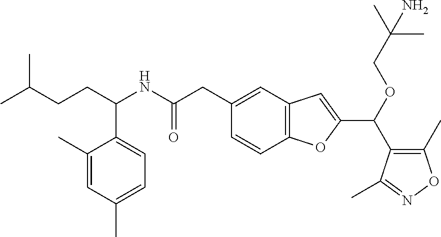 [SMILES: Cc1ccc(C(CCC(C)C)NC(=O)Cc2ccc3oc(C(OCC(C)(C)N)c4c(C)noc4C)cc3c2)c(C)c1]

The title compound was synthesized from 2-(2-(chloro(3,5-dimethylisoxazol-4-yl)methyl)benzofuran-5-yl)-N-(1-(2,4-dimethylphenyl)-4-methylpentyl)acetamide and 2-amino-2-methylpropan-1-ol following essentially the procedure of Example 28 (b) (19 mg, yield: 24%). LCMSA022: 582.0 [M+Na]⁺; Rt=1.533 min. ¹H NMR (MeOD, 400 MHz): δ 7.46 (s, 1H), 7.44 (s, 1H), 7.22 (d, J=8.8 Hz, 1H), 7.05 (d, J=7.6 Hz, 1H), 6.89 (s, 1H), 6.85 (d, J=8.4 Hz, 1H), 6.83 (s, 1H), 5.94 (s, 1H), 4.91 (t, J=7.6 Hz, 1H), 3.75 (d, J=12 Hz, 1H), 3.54-3.49 (m, 3H), 2.46 (s, 3H), 2.25 (s, 3H), 2.18 (s, 3H), 2.15 (s, 1H), 1.63-1.57 (m, 2H), 1.44-1.38 (m, 1H), 1.42-1.37 (m, 1H), 1.34 (s, 3H), 1.10 (s, 3H), 1.08-0.99 (m, 2H), 0.72 (t, J=6.4 Hz, 6H).

### Examples 75 and 76

### 2-(2-((R)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide and 2-(2-((S)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide

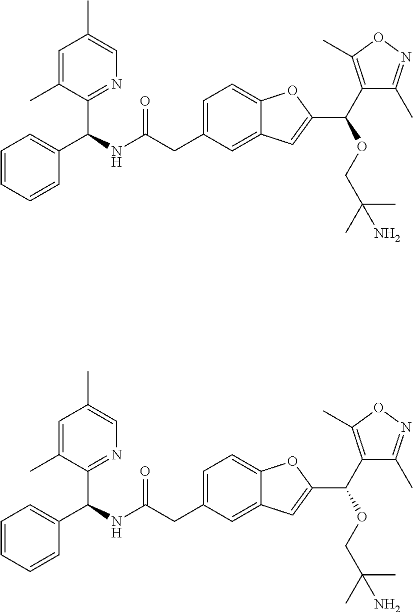 [SMILES: Cc1cnc([C@@H](NC(=O)Cc2ccc3oc([C@H](OCC(C)(C)N)c4c(C)noc4C)cc3c2)c2ccccc2)c(C)c1 ; Cc1cnc([C@@H](NC(=O)Cc2ccc3oc([C@@H](OCC(C)(C)N)c4c(C)noc4C)cc3c2)c2ccccc2)c(C)c1]

### a) 2-(2-((3,5-dimethylisoxazol-4-yl)(hydroxy)methyl)benzofuran-5-yl)-N—((S)-(3,5-dimethylpyridin-2-yl)(phenyl)methyl)acetamide

The title compound was synthesized from (S)-(3,5-dimethylpyridin-2-yl)(phenyl)methanamine and 2-(2-((3,5-dimethylisoxazol-4-yl)(hydroxy)methyl)benzofuran-5-yl)acetic acid following essentially the procedure of Example 2 (b) (600 mg, yield>100%). LC-MSA011: 496.1[M+H]⁺; Rt=1.97 min

### b) 2-(2-((2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide

The title compound was synthesized from 2-(2-((3,5-dimethylisoxazol-4-yl)(hydroxy)methyl)benzofuran-5-yl)-N—((S)-(3,5-dimethylpyridin-2-yl)(phenyl)methyl)acetamide and 2-amino-2-methylpropan-1-ol following essentially the procedure of Example 63 (b) (460 mg, yield>100%). LC-MSA011: 567.3 [M+H]⁺; Rt=2.11 min.

### c) 2-(2-((R)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide and 2-(2-((S)-(2-amino-2-methylpropoxy)(3,5-dimethylisoxazol-4-yl)methyl)benzofuran-5-yl)-N—((S)-(3,5-dimethylpyridin-2-yl)(phenyl)methyl)acetamide

2-(2-((2-amino-2-methylpropoxy)(3,5-dimethylisoxazol-4-yl)methyl)benzofuran-5-yl)-N-((S)-(3,5-dimethylpyridin-2-yl)(phenyl)methyl)acetamide (480 mg) was resolved using the following method:

Instrument: Thar SFC Prep 80 (Thar Technologies, Waters); Column: ChiralCel OD-H, 30 mm I.D.×250 mm Length, 5 μm (Daicel Chemical Industries Co., Ltd); Column Temperature: 35° C.; Mobile Phase: CO₂/MeOH/DEA=70/30/0.5; Flow rate: 70 g/min; Back Pressure: 100 Bar; Wavelength: 214 nm; Cycle time: 3.2 min; Injection Volume: 5.0 mL; Load per injection: 45.8 mg

Said resolution yielded 2 diastereomers:
peak 1, 60 mg, R_{T} 3.6 min, >99% de
peak 2, 50 mg, R_{T} 4.5 min, >99% de

Peak 1: LC-MSA026: 567.3 [M+H]⁺. Rt=2.01 min; ¹H NMR (MeOD, 400 MHz): δ 8.21 (s, 1H), 7.50 (s, 1H), 7.38 (d, J=8.4 Hz, 2H), 7.28-7.19 (m, 6H), 6.78 (s, 1H), 6.27 (s, 1H), 5.62 (s, 1H), 3.70 (s, 2H), 3.36-3.32 (m, 2H), 2.45 (s, 3H), 2.30 (s, 3H), 2.25 (s, 3H), 2.21 (s, 3H), 1.16 (s, 6H).

Peak 2: LC-MSA024: 567.3 [M+H]⁺. Rt=1.35 min; ¹H NMR (MeOD, 400 MHz): δ 8.21 (s, 1H), 7.50 (s, 1H), 7.38 (d, J=8.4 Hz, 2H), 7.28-7.19 (m, 6H), 6.78 (s, 1H), 6.27 (s, 1H), 5.62 (s, 1H), 3.70 (s, 2H), 3.36-3.32 (m, 2H), 2.45 (s, 3H), 2.30 (s, 3H), 2.25 (s, 3H), 2.21 (s, 3H), 1.16 (s, 6H).

### Example 77

### 2-(2-((azetidin-3-yloxy)(3,5-dimethylisoxazol-4-yl)methyl)benzofuran-5-yl)-N-((2,4-dimethylphenyl)(phenyl)methyl)acetamide

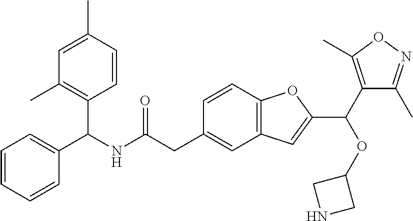 [SMILES: Cc1ccc(C(NC(=O)Cc2ccc3oc(C(OC4CNC4)c4c(C)noc4C)cc3c2)c2ccccc2)c(C)c1]

### a) tert-butyl 3-((3,5-dimethylisoxazol-4-yl)(5-(2-(((2,4-dimethylphenyl)(phenyl)methyl)amino)-2-oxoethyl)benzofuran-2-yl)methoxy)azetidine-1-carboxylate

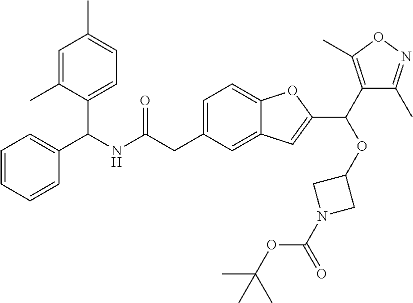 [SMILES: Cc1ccc(C(NC(=O)Cc2ccc3oc(C(OC4CN(C(=O)OC(C)(C)C)C4)c4c(C)noc4C)cc3c2)c2ccccc2)c(C)c1]

The title compound was synthesized from 2-(2-(chloro(3,5-dimethylisoxazol-4-yl)methyl)benzofuran-5-yl)-N-((2,4-dimethylphenyl)(phenyl)methyl)acetamide and tert-butyl 3-hydroxyazetidine-1-carboxylate following essentially the procedure of Example 37 (a) (30 mg, yield 66.7%). LC-MSA011: 672.2[M+H]⁺; Rₜ=1.98 min, purity 91.92% (254 nm).

### b) 2-(2-((azetidin-3-yloxy)(3,5-dimethylisoxazol-4-yl)methyl)benzofuran-5-yl)-N-((2,4-dimethylphenyl)(phenyl)methyl)acetamide

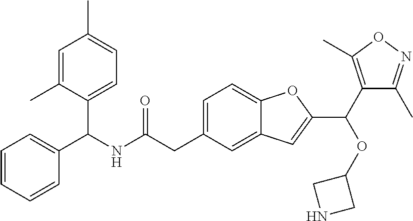 [SMILES: Cc1ccc(C(NC(=O)Cc2ccc3oc(C(OC4CNC4)c4c(C)noc4C)cc3c2)c2ccccc2)c(C)c1]

The title compound was synthesized from tert-butyl 3((3,5-dimethylisoxazol-4-yl)(5-(2-(((2,4-dimethylphenyl)(phenyl)methyl)amino)-2-oxoethyl)benzofuran-2-yl)methoxy)azetidine-1-carboxylate following essentially the procedure of Example 39 (f) (10 mg, yield 97%). ¹H NMR (MeOD, 400 MHz): δ 7.53 (s, 1H), 7.42 (d, J=8.4 Hz, 1H), 7.30-7.24 (m, 4H), 7.14 (d, J=6.8 Hz, 2H), 6.99-6.93 (m, 3H), 6.80 (s, 1H), 6.29 (s, 1H), 5.75 (s, 3H), 4.70-4.67 (m, 1H), 4.27-4.13 (m, 1H), 4.03-3.92 (m, 2H), 3.68 (s, 2H), 2.45 (s, 3H), 2.28 (s, 3H), 2.27 (s, 3H), 2.17 (s, 3H).

### Examples 78 and 79

### 2-(2-((3,5-dimethylisoxazol-4-yl)(1H-tetrazol-1-yl)methyl)benzofuran-5-yl)-N-((2,4-dimethylphenyl)(phenyl)methyl)acetamide and 2-(2-((3,5-dimethylisoxazol-4-yl)(2H-tetrazol-2-yl)methyl)benzofuran-5-yl)-N-((2,4-dimethylphenyl)(phenyl)methyl)acetamide

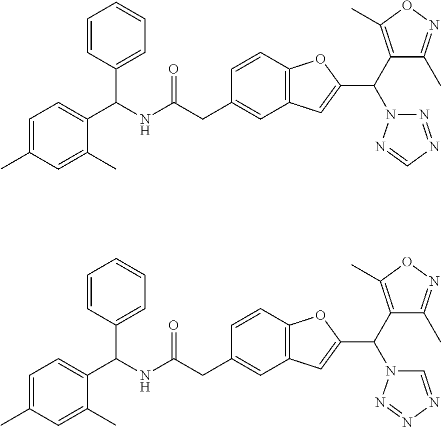

The title compound was synthesized from 2-(2-(chloro(3,5-dimethylisoxazol-4-yl)methyl)benzofuran-5-yl)-N-((2,4-dimethylphenyl)(phenyl)methyl)acetamide and 2H-tetrazole following essentially the procedure of Example 28 (b):

2-(2-((3,5-dimethylisoxazol-4-yl)(2H-tetrazol-2-yl)methyl)benzofuran-5-yl)-N-((2,4-dimethylphenyl)(phenyl)methyl)acetamide: LC-MSA011: No mass [M+H]⁺, fragment at m/z 477; Rt=1.84 min. ¹H NMR (MeOD, 400 MHz): δ 9.44 (s, 1H), 7.56 (s, 1H), 7.50 (s, 1H), 7.45 (d, J=8.4 Hz, 1H), 7.34-7.24 (m, 4H), 7.14 (d, J=7.2 Hz, 2H), 6.99-6.93 (m, 3H), 6.70 (s, 1H), 3.67 (s, 2H), 2.33 (s, 3H), 2.27 (s, 3H), 2.16 (s, 3H), 2.14 (s, 3H).

2-(2-((3,5-dimethylisoxazol-4-yl)(2H-tetrazol-2-yl)methyl)benzofuran-5-yl)-N-((2,4-dimethylphenyl)(phenyl)methyl)acetamide: LC-MSA011: No mass [M+H]⁺ fragment at m/z 477; Rt=1.89 min. ¹H NMR (MeOD, 400 MHz): δ 8.87 (s, 1H), 7.74 (s, 1H), 7.54 (s, 1H), 7.42 (d, J=8.8 Hz, 1H), 7.32-7.26 (m, 4H), 7.14 (d, J=6.8 Hz, 2H), 6.98-6.93 (m, 3H), 6.64 (s, 1H), 6.28 (s, 1H), 3.68 (s, 2H), 2.27 (s, 3H), 2.24 (s, 3H), 2.18 (s, 3H), 2.16 (s, 3H).

### Examples 80 and 81

### 2-(2-((5-amino-2H-tetrazol-2-yl)(3,5-dimethylisoxazol-4-yl)methyl)benzofuran-5-yl)-N-((2,4-dimethylphenyl)(phenyl)methyl)acetamide and 2-(2-(((1H-tetrazol-5-yl)amino)(3,5-dimethylisoxazol-4-yl)methyl)benzofuran-5-yl)-N-((2,4-dimethylphenyl)(phenyl)methyl)acetamide

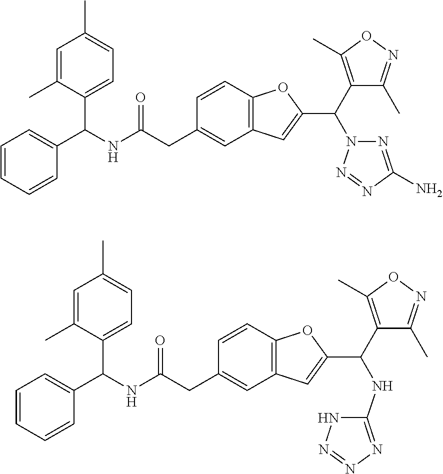 [SMILES: Cc1ccc(C(NC(=O)Cc2ccc3oc(C(c4c(C)noc4C)n4nnc(N)n4)cc3c2)c2ccccc2)c(C)c1 ; Cc1ccc(C(NC(=O)Cc2ccc3oc(C(Nc4nnn[nH]4)c4c(C)noc4C)cc3c2)c2ccccc2)c(C)c1]

The title compound was synthesized from 2-(2-(chloro(3,5-dimethylisoxazol-4-yl)methyl)benzofuran-5-yl)-N-((2,4-dimethylphenyl)(phenyl)methyl)acetamide and 2H-tetrazol-5-amine following essentially the procedure of Example 28 (b):

2-(2-((5-amino-2H-tetrazol-2-yl)(3,5-dimethylisoxazol-4-yl)methyl)benzofuran-5-yl)-N-((2,4-dimethylphenyl)(phenyl)methyl)acetamide: 6 mg, ¹H NMR (MeOD, 400 MHz): δ 7.53 (s, 1H), 7.43 (d, J=8.4 Hz, 1H), 7.30-7.24 (m, 4H), 7.14 (d, J=7.2 Hz, 1H), 6.98-6.91 (m, 4H), 6.48 (s, 1H), 6.28 (s, 1H), 4.65 (s, 1H), 3.67 (s, 2H), 2.29 (s, 3H), 2.27 (s, 3H), 2.15 (s, 3H), 2.13 (s, 3H).

2-(2-(((1H-tetrazol-5-yl)amino)(3,5-dimethylisoxazol-4-yl)methyl)benzofuran-5-yl)-N-((2,4-dimethylphenyl)(phenyl)methyl)acetamide: 8 mg, ¹H NMR (MeOD, 400 MHz): δ 7.53 (s, 1H), 7.43 (d, J=8.4 Hz, 1H), 7.38 (s, 1H), 7.30-7.22 (m, 4H), 7.15 (d, J=7.6 Hz, 2H), 6.98-6.91 (m, 3H), 6.65 (s, 1H), 6.28 (s, 1H), 4.65 (s, 1H), 3.67 (s, 2H), 2.29 (s, 3H), 2.27 (s, 3H), 2.15 (s, 3H), 2.13 (s, 3H).

### Examples 82 and 83

### 2-(2-((R)-(3-aminoazetidin-1-yl)(3,5-dimethylisoxazol-4-yl)methyl)benzofuran-5-yl)-N—((S)-(2,4-dimethylphenyl)(phenyl)methyl)acetamide and 2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide

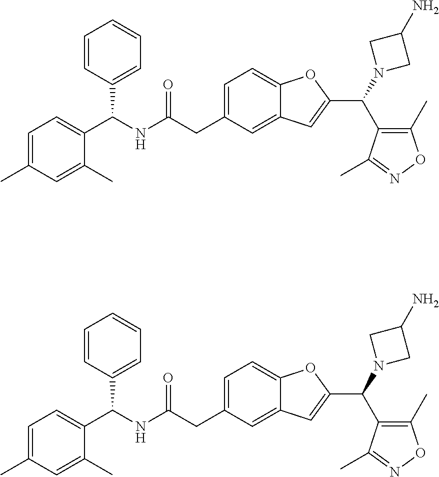

### a) 2-(2-((3-aminoazetidin-1-yl)(3,5-dimethylisoxazol-4-yl)methyl)benzofuran-5-yl)-N—((S)-(2,4-dimethylphenyl)(phenyl)methyl)acetamide

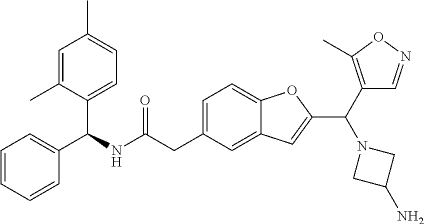 [SMILES: Cc1ccc([C@@H](NC(=O)Cc2ccc3oc(C(c4cnoc4C)N4CC(N)C4)cc3c2)c2ccccc2)c(C)c1]

The title compound was synthesized from (S)-(2,4-dimethylphenyl)(phenyl)methanamine and 2-(2-((3,5-dimethylisoxazol-4-yl)(hydroxy)methyl)benzofuran-5-yl)acetic acid following essentially the procedures of Example 2 (b), Example 28 (a) and Example 38 (a) and (b). ¹H NMR (MeOD, 400 MHz): δ 7.48 (s, 1H), 7.34 (d, J=8.4 Hz, 1H), 7.28-7.20 (m, 4H), 7.14 (d, J=6.8 Hz, 2H), 6.98 (s, 1H), 6.93-6.92 (m, 2H), 6.64 (s, 1H), 6.28 (s, 1H), 4.61 (s, 1H), 3.72-3.70 (m, 1H), 3.65 (s, 2H), 3.53-3.54 (m, 1H), 3.50-3.48 (m, 1H), 2.93-2.92 (m, 1H), 2.84-2.83 (m, 1H), 2.53 (s, 3H), 2.28 (s, 3H), 2.27 (s, 3H), 2.16 (s, 3H).

### b) 2-(2-((R)-(3-aminoazetidin-1-yl)(3,5-dimethylisoxazol-4-yl)methyl)benzofuran-5-yl)-N-((S)-(2,4-dimethylphenyl)(phenyl)methyl)acetamide and 2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide

2-(2-((3-aminoazetidin-1-yl)(3,5-dimethylisoxazol-4-yl)methyl)benzofuran-5-yl)-N—((S)-(2,4-dimethylphenyl)(phenyl)methyl)acetamide (590 mg) was resolved using the following method:

Instrument: SFC80; Column: IC-H, 30×250 mm, 5 μm; Column Temperature: 35° C.; Mobile Phase: CO₂/MeOH/DEA=50/50/0.5; Flow rate: 80 g/min; Back Pressure: 100 Bar; Wavelength: 214 nm; Cycle time: 2.5 min; Injection: 2.0 mL
Said resolution yielded 2 diastereomers:
peak 1, 85 mg, R_{T} 4.45 min, 91% de
peak 2, 93 mg, R_{T} 5.15 min, 95% de

### Examples 84 and 85

### 2-(2-((R)-(3-aminoazetidin-1-yl)(pyridin-4-yl)methyl)benzofuran-5-yl)-N—((S)-(2,4-dimethylphenyl)(phenyl)methyl)acetamide and 2-(2-((S)-(3-aminoazetidin-1-yl)(pyridin-4-yl)methyl)benzofuran-5-yl)-N—((S)-(2,4-dimethylphenyl)(phenyl)methyl)acetamide

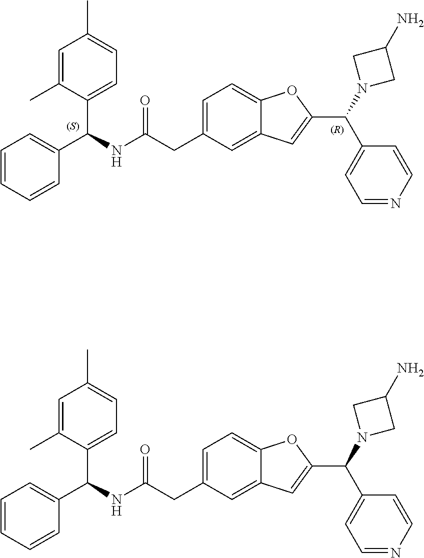

### a) 2-(2-((3-aminoazetidin-1-yl)(pyridin-4-yl)methyl)benzofuran-5-yl)-N—((S)-(2,4-dimethylphenyl)(phenyl)methyl)acetamide

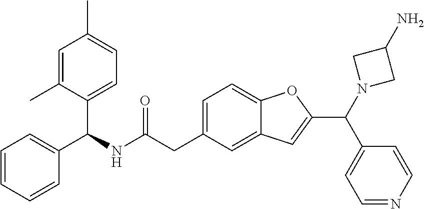 [SMILES: Cc1ccc([C@@H](NC(=O)Cc2ccc3oc(C(c4ccncc4)N4CC(N)C4)cc3c2)c2ccccc2)c(C)c1]

The title compound was synthesized from (S)-(2,4-dimethylphenyl)(phenyl)methanamine and 2-(2-(hydroxy(pyridin-4-yl)methyl)benzofuran-5-yl)acetic acid following essentially the procedures of Example 2 (b), Example 28 (a) and Example 38 (a) and (b). LC-MS (022): 531.2 [M+H]⁺; Rt: 1.51 min

### b) 2-(2-((R)-(3-aminoazetidin-1-yl)(pyridin-4-yl)methyl)benzofuran-5-yl)-N—((S)-(2,4-dimethylphenyl)(phenyl)methyl)acetamide and 2-(2-((S)-(3-aminoazetidin-1-yl)(pyridin-4-yl)methyl)benzofuran-5-yl)-N—((S)-(2,4-dimethylphenyl)(phenyl)methyl)acetamide

2-(2-((3-aminoazetidin-1-yl)(pyridin-4-yl)methyl)benzofuran-5-yl)-N—((S)-(2,4-dimethylphenyl)(phenyl)methyl)acetamide (200 mg) was resolved using the following method:

Instrument: Thar SFC Prep 80 (Thar Technologies, Waters); Column: ChiralCel OJ-H, 30 mm I.D.×250 mm Length, 5 μm (Daicel Chemical Industries Co., Ltd); Column Temperature: 35° C.; Mobile Phase: CO₂/MeOH/DEA=70/30/0.1; Flow rate: 80 g/min; Back Pressure: 100 Bar; Wavelength: 214 nm; Cycle time: 6.7 min; Injection Volume: 4.0 mL; Load per injection: 26 mg

Said resolution yielded 2 diastereomers:
peak 1, 2 mg, R_{T} 2.9 min, >99% de
peak 2, 2 mg, R_{T} 4.6 min, 90% de

Peak 1: LC-MS (012): 531.2 [M+H]⁺; Rt: 1.35 min ¹H NMR (CDCL3, 400 MHz): δ 8.50 (d, J=4.8 Hz, 2H), 7.34-6.97 (m, 8H), 6.95-6.64 (m, 5H), 6.53 (s, 1H), 6.28 (d, J=8.4 Hz, 1H), 5.85 (d, J=6.8 Hz, 1H), 4.42 (s, 1H), 3.66-3.51 (m, 5H), 2.80-2.66 (m, 2H), 2.20 (s, 3H), 2.12 (s, 3H).

Peak 2: LC-MS (012): 531.2 [M+H]⁺; Rt: 1.34 min ¹H NMR (CDCL3, 400 MHz): δ 8.50 (d, J=4.8 Hz, 2H), 7.34-7.04 (m, 8H), 6.97-6.64 (m, 5H), 6.53 (s, 1H), 6.28 (d, J=8.0 Hz, 1H), 5.85 (d, J=7.6 Hz, 1H), 4.42 (s, 1H), 3.66-3.50 (m, 5H), 2.82-2.66 (m, 2H), 2.20 (s, 3H), 2.12 (s, 3H).

### Examples 86 and 87

### ethyl 2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetate and ethyl 2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetate

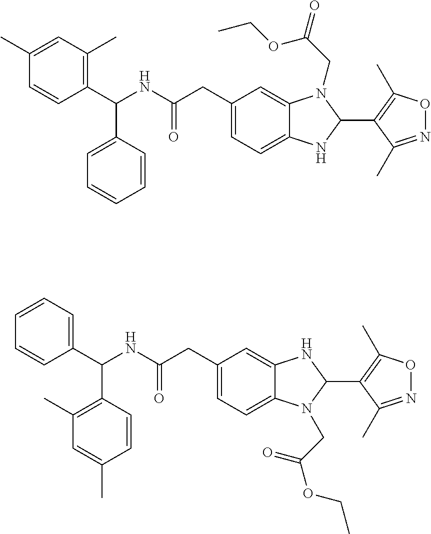 [SMILES: CCOC(=O)CN1c2cc(CC(=O)NC(c3ccccc3)c3ccc(C)cc3C)ccc2NC1c1c(C)noc1C ; CCOC(=O)CN1c2ccc(CC(=O)NC(c3ccccc3)c3ccc(C)cc3C)cc2NC1c1c(C)noc1C]

### a) 2-(2-(3,5-dimethylisoxazol-4-yl)-1H-benzo[d]imidazol-6-yl)acetic acid

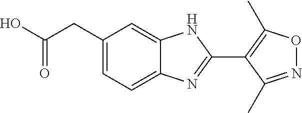 [SMILES: Cc1noc(C)c1-c1nc2ccc(CC(=O)O)cc2[nH]1]

The title compound was synthesized from ethyl 2-(2-(3,5-dimethylisoxazol-4-yl)-1H-benzo[d]imidazol-6-yl)acetate following essentially the procedure of Example 31 (b) (230 mg, crude). LC-MS (010): 272.1 [M+H]⁺; Rₜ=1.91 min. purity: 66% (254 nm)

### b) 2-(2-(3,5-dimethylisoxazol-4-yl)-1H-benzo[d]imidazol-6-yl)acetyl chloride

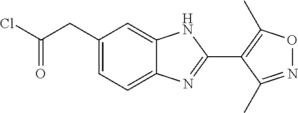 [SMILES: Cc1noc(C)c1-c1nc2ccc(CC(=O)Cl)cc2[nH]1]

SOCl₂ (106 mg, 0.9 mmol) was added to a solution of 2-(2-(3,5-dimethylisoxazol-4-yl)-1H-benzo[d]imidazol-6-yl)acetic acid (230 mg, 0.85 mmol) in dichloromethane (20 mL). The reaction mixture was stirred at rt for 3 h, and then concentrated under reduced pressure to yield 2-(2-(3,5-dimethylisoxazol-4-yl)-1H-benzo[d]imidazol-6-yl)acetyl chloride as a yellow solid (261 mg), which was carried through without any purification.

### c) benzyl 2-(2-(3,5-dimethylisoxazol-4-yl)-1H-benzo[d]imidazol-6-yl)acetate

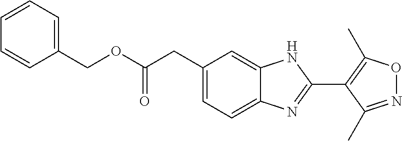 [SMILES: Cc1noc(C)c1-c1nc2ccc(CC(=O)OCc3ccccc3)cc2[nH]1]

Phenylmethanol (97 mg, 0.9 mmol) was added to a solution of 2-(2-(3,5-dimethylisoxazol-4-yl)-1H-benzo[d]imidazol-6-yl)acetyl chloride (261 mg, 0.85 mmol) in dichloromethane (20 mL). The reaction mixture was stirred at rt for 3 h, and then washed with saturated solution of sodium hydrogen carbonate (5 mL×3), dried over anhydrous sodium sulfate and concentrated under reduced pressure. The resulting residue was purified by column chromatography (petroleum ether/EtOAc: 5/1) to yield benzyl 2-(2-(3,5-dimethylisoxazol-4-yl)-1H-benzo[d]imidazol-6-yl)acetate (170 mg, yield: 55%, 3 steps) as a white solid. LC-MS (010): 362.2 [M+H]⁺; Rₜ=1.69 min. purity: 100% (214 nm)

### d) benzyl 2-(2-(3,5-dimethylisoxazol-4-yl)-1-(2-ethoxy-2-oxoethyl)-1H-benzo[d]imidazol-6-yl)acetate and benzyl 2-(2-(3,5-dimethylisoxazol-4-yl)-1-(2-ethoxy-2-oxoethyl)-1H-benzo[d]imidazol-5-yl)acetate

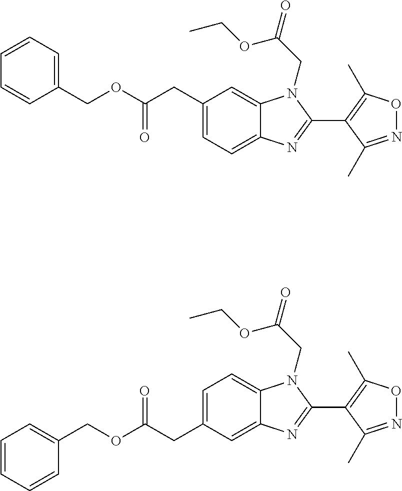 [SMILES: CCOC(=O)Cn1c(-c2c(C)noc2C)nc2ccc(CC(=O)OCc3ccccc3)cc21 ; CCOC(=O)Cn1c(-c2c(C)noc2C)nc2cc(CC(=O)OCc3ccccc3)ccc21]

Ethyl 2-bromoacetate (94 mg, 0.562 mmol) was added to a solution of benzyl 2-(2-(3,5-dimethylisoxazol-4-yl)-1H-benzo[d]imidazol-6-yl)acetate (170 mg, 0.47 mmol) and triethylamine (50.5 mg, 0.5 mmol) in tetrahydrofuran (20 mL). The reaction mixture was stirred at 80° C. overnight, washed with brine (5 mL×3), dried over anhydrous sodium sulfate and concentrated under reduced pressure. The resulting residue was purified by chiral HPLC to obtain compound benzyl 2-(2-(3,5-dimethylisoxazol-4-yl)-1-(2-ethoxy-2-oxoethyl)-1H-benzo[d]imidazol-6-yl)acetate (78 mg) and benzyl 2-(2-(3,5-dimethylisoxazol-4-yl)-1-(2-ethoxy-2-oxoethyl)-1H-benzo[d]imidazol-5-yl)acetate (72 mg) as white solids. LC-MS (010): 362.2 [M+H]⁺

### e) 2-(2-(3,5-dimethylisoxazol-4-yl)-3-(2-ethoxy-2-oxoethyl)-2,3-dihydro-1H-benzo[d]imidazol-5-yl)acetic acid

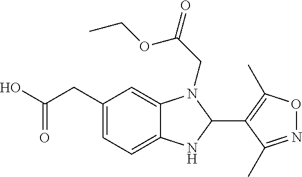 [SMILES: CCOC(=O)CN1c2cc(CC(=O)O)ccc2NC1c1c(C)noc1C]

The mixture of benzyl 2-(2-(3,5-dimethylisoxazol-4-yl)-1-(2-ethoxy-2-oxoethyl)-1H-benzo[d]imidazol-6-yl)acetate (78 mg, 0.17 mmol) and Pd(OH)₂ (1 mg) in ethanol (10 mL) was stirred under H₂ atmosphere at rt for 1 h. The reaction mixture was then filtered and the filtrate was concentrated under reduced pressure to give 2-(2-(3,5-dimethylisoxazol-4-yl)-3-(2-ethoxy-2-oxoethyl)-2,3-dihydro-1H-benzo[d]imidazol-5-yl)acetic acid (50 mg, 82%), which was carried through with out further purification. LC-MS (010): 360.1 [M+H]⁺; Rt=1.42 min.

### f) 2-(2-(3,5-dimethylisoxazol-4-yl)-1-(2-ethoxy-2-oxoethyl)-2,3-dihydro-1H-benzo[d]imidazol-5-yl)acetic acid

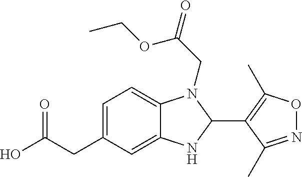 [SMILES: CCOC(=O)CN1c2ccc(CC(=O)O)cc2NC1c1c(C)noc1C]

The title compound was synthesized from benzyl 2-(2-(3,5-dimethylisoxazol-4-yl)-1-(2-ethoxy-2-oxoethyl)-1H-benzo[d]imidazol-5-yl)acetate following essentially the procedure of Examples 86 and 87 (e) (55 mg, 92%). LC-MS (010): 360.1 [M+H]⁺; Rₜ=1.41 min.

### g) ethyl 2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetate

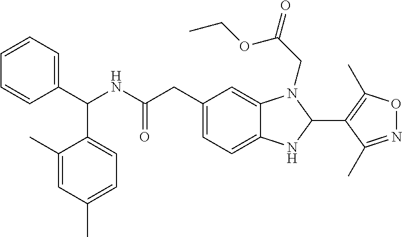 [SMILES: CCOC(=O)CN1c2cc(CC(=O)NC(c3ccccc3)c3ccc(C)cc3C)ccc2NC1c1c(C)noc1C]

The title compound was synthesized from benzyl (2,4-dimethylphenyl)(phenyl)methanamine hydrochloride and 2-(2-(3,5-dimethylisoxazol-4-yl)-3-(2-ethoxy-2-oxoethyl)-2,3-dihydro-1H-benzo[d]imidazol-5-yl)acetic acid following essentially the procedure of Examples 2 (b) (14 mg, yield: 20%). LC-MS022: 553.3 [M+H]⁺; Rt=1.493 min. ¹H NMR (CDCl₃, 400 MHz): δ 10.79 (d, J=6.4 Hz, 1H), 7.75 (d, J=8.4 Hz, 1H), 7.25-7.17 (m, 4H), 7.06 (d, J=7.6 Hz, 2H), 6.97 (s, 1H), 6.91-6.87 (m, 1H), 6.79-6.75 (m, 1H), 6.38 (d, J=8.0 Hz, 1H), 6.05 (d, J=8.0 Hz, 1H), 5.61 (d, J=6.0 Hz, 1H), 4.74 (s, 2H), 4.17 (q, J=7.2 Hz, 2H), 3.76 (s, 2H), 2.27 (s, 3H), 2.21 (s, 3H), 1.86 (s, 3H), 1.80 (s, 3H), 1.24 (t, J=7.2 Hz, 3H).

### h) ethyl 2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetate

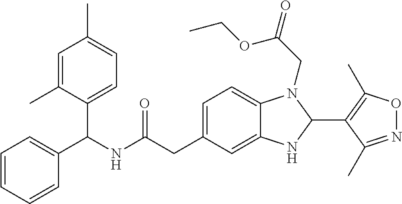 [SMILES: CCOC(=O)CN1c2ccc(CC(=O)NC(c3ccccc3)c3ccc(C)cc3C)cc2NC1c1c(C)noc1C]

The title compound was synthesized from benzyl (2,4-dimethylphenyl)(phenyl)methanamine hydrochloride and 2-(2-(3,5-dimethylisoxazol-4-yl)-1-(2-ethoxy-2-oxoethyl)-2,3-dihydro-1H-benzo[d]imidazol-5-yl)acetic acid following essentially the procedure of Examples 2 (b) (8 mg, yield: 15%). LC-MS022: 553.3 [M+H]⁺; Rₜ=1.513 min. ¹H NMR (CDCl₃, 400 MHz): δ 10.73 (d, J=5.6 Hz, 1H), 7.61 (s, 1H), 7.18-7.13 (m, 4H), 7.00 (d, J=6.8 Hz, 2H), 6.89 (s, 1H), 6.82 (d, J=7.6 Hz, 1H), 6.71 (dd, J=8.0, 2.0 Hz, 1H), 6.31 (d, J=8.4 Hz, 1H), 5.96 (d, J=8.0 Hz, 1H), 5.47 (s, 1H), 4.71 (s, 2H), 4.14 (q, J=7.2 Hz, 2H), 3.71 (s, 2H), 2.20 (s, 3H), 2.14 (s, 3H), 1.79 (s, 3H), 1.74 (s, 3H), 1.18 (t, J=7.2 Hz, 3H).

### Example 88

### 2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetic acid

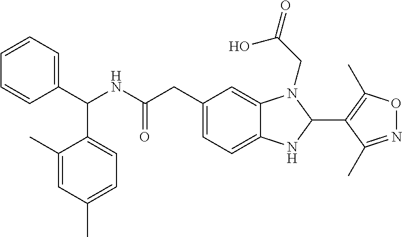 [SMILES: Cc1ccc(C(NC(=O)Cc2ccc3c(c2)N(CC(=O)O)C(c2c(C)noc2C)N3)c2ccccc2)c(C)c1]

The title compound was synthesized from ethyl 2-(2-(3,5-dimethylisoxazol-4-yl)-6-(2-(((2,4-dimethylphenyl)(phenyl)methyl)amino)-2-oxoethyl)-2,3-dihydro-1H-benzo[d]imidazol-1-yl)acetate following essentially the procedure of Examples 31 (b) (8 mg, yield: 67%). LCMSA011: 525.0 [M+H]⁺; Rₜ=1.432 min ¹H NMR (MeOD, 400 MHz): δ 7.59 (d, J=8.0 Hz, 1H), 7.49 (s, 1H), 7.84 (d, J=8.4 Hz, 1H), 7.22-7.12 (m, 3H), 7.06 (d, J=7.2 Hz, 2H), 6.90-6.82 (m, 3H), 6.21-6.19 (m, 1H), 5.25 (t, J=4.8 Hz, 1H), 4.83 (s, 2H), 3.70 (s, 2H), 2.17 (s, 3H), 2.09 (s, 3H), 1.75 (d, J=7.2 Hz, 6H).

### Example 89

### 2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetic acid

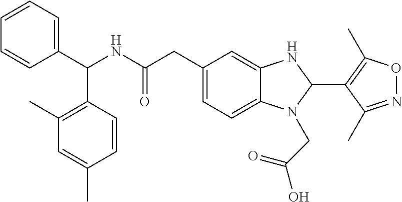 [SMILES: Cc1ccc(C(NC(=O)Cc2ccc3c(c2)NC(c2c(C)noc2C)N3CC(=O)O)c2ccccc2)c(C)c1]

The title compound was synthesized from ethyl 2-(2-(3,5-dimethylisoxazol-4-yl)-5-(2-(((2,4-dimethylphenyl)(phenyl)methyl)amino)-2-oxoethyl)-2,3-dihydro-1H-benzo[d]imidazol-1-yl)acetate following essentially the procedure of Examples 31 (b) (2 mg, yield: 9%). LCMSA012: 525.2 [M+H]⁺; Rₜ=1.070 min. ¹H NMR (MeOD, 400 MHz): δ 8.97 (d, J=7.6 Hz, 1H), 7.70 (s, 1H), 7.59 (d, J=8.4 Hz, 1H), 7.47 (d, J=8.4 Hz, 1H), 7.31-7.24 (m, 3H), 7.16 (d, J=7.6 Hz, 2H), 6.99-6.93 (m, 3H), 6.31-6.29 (m, 1H), 5.35 (t, J=4.8 Hz, 1H), 4.83 (s, 2H), 3.78 (s, 2H), 2.28 (s, 3H), 2.18 (s, 3H), 1.85 (d, J=6.8 Hz, 6H).

### Example 90

### 2-(2-((3,5-dimethylisoxazol-4-yl)(methylsulfonyl)methyl)benzofuran-5-yl)-N-((2,4-dimethylphenyl)(phenyl)methyl)acetamide

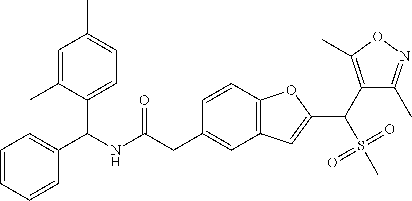 [SMILES: Cc1ccc(C(NC(=O)Cc2ccc3oc(C(c4c(C)noc4C)S(C)(=O)=O)cc3c2)c2ccccc2)c(C)c1]

### a) 2-(2-((3,5-dimethylisoxazol-4-yl)(methylthio)methyl)benzofuran-5-yl)-N-((2,4-dimethylphenyl)(phenyl)methyl)acetamide

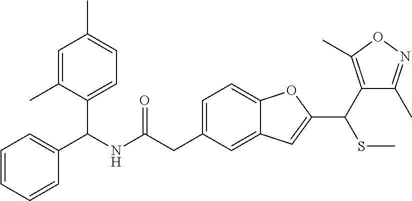 [SMILES: CSC(c1cc2cc(CC(=O)NC(c3ccccc3)c3ccc(C)cc3C)ccc2o1)c1c(C)noc1C]

CH₃CO₂H (11 mg, 0.18 mmol) was added to a solution of sodium methanethiolate (13 mg, 0.18 mmol) in CH₂Cl₂ (5 mL) at 0° C. under N₂. After 30 min, 2-(2-(chloro(3,5-dimethylisoxazol-4-yl)methyl)benzofuran-5-yl)-N-((2,4-dimethylphenyl)(phenyl)methyl)acetamide (26 mg, 0.05 mmol) and Zn1₂ (32 mg, 0.10 mmol) were added also at 0° C. and under N₂. The mixture was stirred at rt for 5 h. Then water (30 mL) was added to the reaction mixture, and the mixture was extracted with ethyl acetate (30 mL×3). The combined extracts were washed with brine (15 mL×3), dried over Na₂SO₄, and concentrated under reduced pressure to give 2-(2-((3,5-dimethylisoxazol-4-yl)(methylthio)methyl)benzofuran-5-yl)-N-((2,4-dimethylphenyl)(phenyl)methyl)acetamide (40 mg, yield >100%) as a yellow oil, which was carried through without further purification. LC-MSA043: 525.3 [M+H]⁺; Rₜ=2.32 min.

### b) 2-(2-((3,5-dimethylisoxazol-4-yl)(methylsulfonyl)methyl)benzofuran-5-yl)-N-((2,4-dimethylphenyl)(phenyl)methyl)acetamide

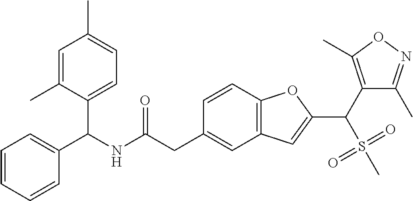 [SMILES: Cc1ccc(C(NC(=O)Cc2ccc3oc(C(c4c(C)noc4C)S(C)(=O)=O)cc3c2)c2ccccc2)c(C)c1]

The title compound was synthesized from 2-(2-((3,5-dimethylisoxazol-4-yl)(methylthio)methyl)benzofuran-5-yl)-N-((2,4-dimethylphenyl)(phenyl)methyl)acetamide following essentially the procedure of Examples 46 (15 mg, yield 35%). LC-MSA011: 557.2[M+H]⁺; Rₜ=1.50 min. ¹H NMR (MeOD, 400 MHz): δ 7.58 (s, 1H), 7.48 (d, J=8.8 Hz, 1H), 7.33-7.24 (m, 5H), 7.16-7.13 (m, 2H), 7.06 (s, 1H), 7.00-6.94 (s, 3H), 6.29 (s, 1H), 3.69 (s, 2H), 3.16 (s, 3H), 2.56 (s, 3H), 2.33 (s, 3H), 2.27 (s, 3H), 2.17 (s, 3H).

### Example 91

### 2-(2-((3,5-dimethylisoxazol-4-yl)(methylsulfonamido)methyl)benzofuran-5-yl)-N-((2,4-dimethylphenyl)(phenyl)methypacetamide

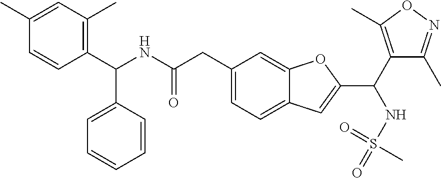

### a) 2-(2-(amino(3,5-dimethylisoxazol-4-yl)methyl)benzofuran-5-yl)-N-((2,4-dimethylphenyl)(phenyl)methyl)acetamide

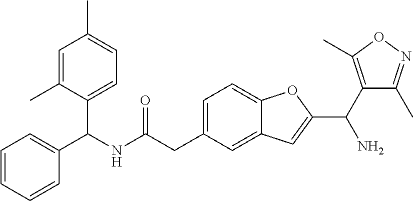 [SMILES: Cc1ccc(C(NC(=O)Cc2ccc3oc(C(N)c4c(C)noc4C)cc3c2)c2ccccc2)c(C)c1]

A mixture of 2-(2-(chloro(3,5-dimethylisoxazol-4-yl)methyl)benzofuran-5-yl)-N-((2,4-dimethylphenyl)(phenyl)methyl)acetamide (20 mg, 0.04 mmol) in an aqueous ammonia solution (5 mL) was stirred at rt for 3 h. The mixture was extracted with CH₂Cl₂ (3×20 mL). The combined organic layers were washed with water and dried over Na₂SO₄. The organic solvent was concentrated to residue which was purified by preparatory HPLC using 10-100% water/acetonitrile with 0.1% TFA to obtain the title compound (8 mg, 41%). LCMS-P1: 494 [M+H]⁺; Rₜ=1.462 min. ¹H NMR (500 MHz, CDCl₃) δ ppm 7.25-6.98 (m, 8H), 6.87-6.15 (m, 6H), 5.31 (s, 1H), 3.47 (s, 2H), 2.17 (s, 6H), 2.05 (s, 3H), 1.99 (s, 3H).

### b) 2-(2-((3,5-dimethylisoxazol-4-yl)(methylsulfonamido)methyl)benzofuran-5-yl)-N-((2,4-dimethylphenyl)(phenyl)methyl)acetamide

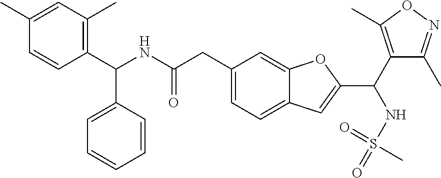

Methanesulfonyl chloride (11.4 mg, 0.1 mmol) was added to a solution of 2-(2-(amino(3,5-dimethylisoxazol-4-yl)methyl)benzofuran-5-yl)-N-((2,4-dimethylphenyl)(phenyl)methyl)acetamide (49.3 mg, 0.1 mmol) and Et₃N (20.2 mg, 0.2 mmol) in 5 mL of CH₂Cl₂ at 0° C. and the mixture was stirred for 2 h. The reaction mixture was then concentrated under reduced pressure. The resulting residue was purified by Pre-HPLC to obtain 2-(2-((3,5-dimethylisoxazol-4-yl)(methylsulfonamido)methyl)benzofuran-5-yl)-N-((2,4-dimethylphenyl)(phenyl)methyl)acetamide (12 mg, 21%). LC-MS024: 572.2.0 [M+H]⁺; Rt=1.47 min. ¹H NMR (CDCL3, 400 MHz): δ7.35-7.11 (m, 6H), 7.01-6.71 (m, 5H), 6.50 (s, 1H), 6.27 (d, J=8.0 Hz, 1H), 5.99 (d, J=7.6 Hz, 1H), 5.73-5.58 (m, 2H), 3.60 (s, 2H), 2.73 (s, 3H), 2.46 (s, 3H), 2.20 (s, 3H), 2.14 (s, 3H), 2.11 (s, 3H).

### Example 92

### N—((S)-(2,4-dimethylphenyl)(phenyl)methyl)-2-(2-(1-hydroxy-1-(pyridin-4-yOethyl)benzofuran-5-yl)acetamide

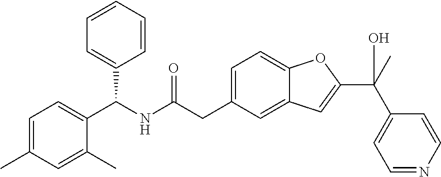 [SMILES: Cc1ccc([C@@H](NC(=O)Cc2ccc3oc(C(C)(O)c4ccncc4)cc3c2)c2ccccc2)c(C)c1]

The title compound was synthesized following essentially the procedure of Examples 46 except using (S)-(2,4-dimethylphenyl)(phenyl)methanamine instead of the racemic amine LC-MS (036): 491.2 [M+H]⁺; Rt: 1.41 min. ¹H NMR (CDCL3, 400 MHz): δ 8.53 (s, 2H), 7.66 (s, 2H), 7.36-7.07 (m, 7H), 7.00-6.62 (m, 6H), 6.28 (d, J=8.0 Hz, 1H), 5.99 (d, J=7.6 Hz, 1H), 3.60 (s, 2H), 2.20 (s, 3H), 2.13 (s, 3H), 1.89 (s, 3H).

### Examples 93 and 94

### 4-((S)-1-(5-(2-(((S)-(2,4-dimethylphenyl)(phenyl)methyl)amino)-2-oxoethyl)benzofuran-2-yl)-1-hydroxyethyl)pyridine 1-oxide and 4-((R)-1-(5-(2-((S)-(2,4-dimethylphenyl)(phenyl)methyl)amino)-2-oxoethyl)benzofuran-2-yl)-1-hydroxyethyl)pyridine 1-oxide

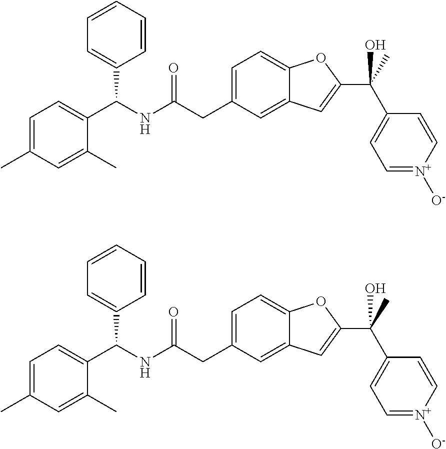 [SMILES: Cc1ccc([C@@H](NC(=O)Cc2ccc3oc([C@@](C)(O)c4cc[n+]([O-])cc4)cc3c2)c2ccccc2)c(C)c1 ; Cc1ccc([C@@H](NC(=O)Cc2ccc3oc([C@](C)(O)c4cc[n+]([O-])cc4)cc3c2)c2ccccc2)c(C)c1]

4-(1-(5-(2-(((S)-(2,4-dimethylphenyl)(phenyl)methyl)amino)-2-oxoethyl)benzofuran-2-yl)-1-hydroxyethyl)pyridine 1-oxide (260 mg) was resolved using the following method: Instrument: Thar SFC Prep 80 (Thar Technologies, Waters); Column: ChiralPak AD-H, 30 mm I.D.×250 mm Length, 5 μm (Daicel Chemical Industries Co., Ltd); Column Temperature: 35° C.; Mobile Phase: CO₂/MeOH=50/50; Flow rate: 60 g/min; Back Pressure: 100 Bar; Wavelength: 214 nm; Cycle time: 13.0 min; Injection Volume: 4.5 mL; Load per injection: 28 mg; Feed solution: 280 mg dissolved in 45 mL MeOH

Said resolution yielded 2 diastereomers:
peak 1, 45 mg, RT 3.2 min, >99% de
peak 2, 40 mg, RT 4.5 min, >99% de

Peak 1: LC-MS (036): 507.2 [M+H]⁺; Rt: 1.56 min. ¹H NMR (CDCL3, 400 MHz): δ 7.92 (d, J=6.4 Hz, 2H), 7.35-7.18 (m, 7H), 7.08-6.80 (m, 6H), 6.58 (s, 1H), 6.41-6.34 (m, 2H), 5.20 (s, 1H), 3.60 (s, 2H), 2.26 (s, 3H), 2.19 (s, 3H), 1.89 (s, 3H).

Peak 2: LC-MS (036): 507.2 [M+H]⁺; Rt: 1.56 min. ¹H NMR (CDCL3, 400 MHz): δ 7.90 (d, J=6.4 Hz, 2H), 7.35-7.18 (m, 7H), 7.08-6.81 (m, 6H), 6.57 (s, 1H), 6.47-6.34 (m, 2H), 5.29 (s, 1H), 3.60 (s, 2H), 2.26 (s, 3H), 2.19 (s, 3H), 1.88 (s, 3H).

### Example 94

### N—((S)-(2,4-dimethylphenyl)(phenyl)methyl)-2-(2-(1-hydroxy-1-(pyrimidin-4-yl)ethyl)furo[3,2-b]pyridin-5-yl)acetamide

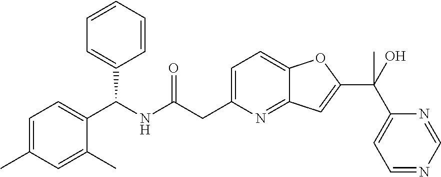 [SMILES: Cc1ccc([C@@H](NC(=O)Cc2ccc3oc(C(C)(O)c4ccncn4)cc3n2)c2ccccc2)c(C)c1]

### a) N-methoxy-N-methylpyrimidine-4-carboxamide

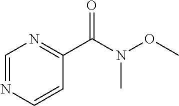

The title compound was synthesized from pyrimidine-4-carboxylic acid and N,O-dimethylhydroxylamine following essentially the procedure of Examples 47 (a) (4.89 g, yield: 59%). LC-MS020: 168.1[M+H]⁺; Rt=0.85 min; Purity: 42.68% (214 nm).

### b) 1-(pyrimidin-4-yl)-3-(triisopropylsilyl)prop-2-yn-1-one

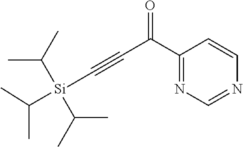 [SMILES: CC(C)[Si](C#CC(=O)c1ccncn1)(C(C)C)C(C)C]

Ethynyltriisopropylsilane (1.28 g, 9.02 mol) and LHMDS (9.02 mmol, 1M, 9.02 mL) were added to a solution of N-methoxy-N-methylpyrimidine-4-carboxamide (1.16 g, 6.94 mol) in 30 mL of THF, and the mixture was stirred at −78° C. for 2 h. The reaction mixture was then quenched with aqueous NaHCO₃ (30 mL) and extracted with EtOAc (30 mL×3). The combined extracts were washed with brine (20 mL), dried over Na₂SO₄, and concentrated under reduced pressure to obtain 1-(pyrimidin-4-yl)-3-(triisopropylsilyl)prop-2-yn-1-one as a brown oil (1.6 g, crude). LCMSA020: 289.2 [M+H]⁺; Rt=2.01 min

### c) 2-(pyrimidin-4-yl)-4-(triisopropylsilyl)but-3-yn-2-ol

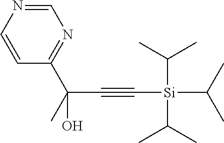 [SMILES: CC(C)[Si](C#CC(C)(O)c1ccncn1)(C(C)C)C(C)C]

The title compound was synthesized from 1-(pyrimidin-4-yl)-3-(triisopropylsilyl)prop-2-yn-1-one following essentially the procedure of Examples 45 (d) (719 mg, crude) and it was carried through without further purification. LCMSA044: 305.2 [M+H]⁺; Rt=1.93 min

### d) 2-(pyrimidin-4-yl)but-3-yn-2-ol

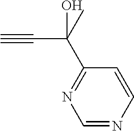

TBAF (2.83 mmol, 1M, 2.9 mL) was added to a solution of 2-(pyrimidin-4-yl)-4-(triisopropylsilyl)but-3-yn-2-ol (719 mg, 2.36 mol) in 20 mL of THF, and the mixture was stirred at rt for 2 h. The reaction mixture then was quenched with water (20 mL) and it was extracted with EtOAc (20 mL×3). The combined extracts were washed with brine (20 mL), dried over Na₂SO₄, and concentrated under reduced pressure to obtain 2-(pyrimidin-4-yl)but-3-yn-2-ol (908 mg, crude) as brown oil, which was carried through without further purification. LCMSA027: 149.1 [M+H]⁺; Rt=0.47 min.

### e) Ethyl 2-(5-hydroxy-6-iodopyridin-2-yl)acetate

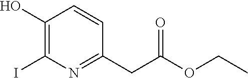

Iodine (716.3 mg, 2.82 mmol) was added to a solution of ethyl 2-(5-hydroxypyridin-2-yl)acetate (510 mg, 2.82 mmol) and sodium carbonate (627.5 mg, 5.92 mmol) in water (20 mL) under nitrogen atmosphere, and the mixture was stirred at rt for 2 h. The reaction mixture was extracted with EtOAc, washed with brine, and dried over sodium sulfate. Removal of solvent gave 600 mg of crude ethyl 2-(5-hydroxy-6-iodopyridin-2-yl)acetate, which was carried through without further purification. LCMS-027: 308.0 [M+H]⁺; Rt: 1.11 min

### f) ethyl 2-(2-(1-hydroxy-1-(pyrimidin-4-yl)ethyl)furo[3,2-b]pyridin-5-yl)acetate

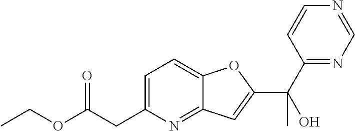 [SMILES: CCOC(=O)Cc1ccc2oc(C(C)(O)c3ccncn3)cc2n1]

2-(Pyrimidin-4-yl)but-3-yn-2-ol (908 mg, 2.36 mmol), CuI (28 mg, 0.15 mmol), and Pd(Ph₃P)₂Cl₂ (87 mg, 0.12 mmol) were added to a solution of ethyl 2-(5-hydroxy-6-iodopyridin-2-yl)acetate (361.08 mg, 1.18 mmol) in 20 mL of Et₃N. The resulting solution was heated to reflux under N₂ for 3 h. After cooling to rt, the solution was filtered through a pad of Celite. The filtrate was concentrated under reduced pressure. EtOAc and water were added to the resulting residue and the EtOAc layer was washed with brine (30 mL), dried over anhydrous Na₂SO₄, and concentrated under reduced pressure. The resulting residue was purified by column chromatography (petroleum ether/EtOAc=5/1) to obtain ethyl 2-(2-(1-hydroxy-1-(pyrimidin-4-yl)ethyl)furo[3,2-b]pyridin-5-yl)acetate (263 mg, yield: 38%) as yellow solid. LCMSA020: 328.1 [M+H]⁺; Rt=1.31 min; Purity=73.32% (214 nm).

### g) 2-(2-(1-hydroxy-1-(pyrimidin-4-yl)ethyl)furo[3,2-b]pyridin-5-yl)acetic acid

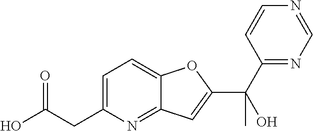 [SMILES: CC(O)(c1ccncn1)c1cc2nc(CC(=O)O)ccc2o1]

The title compound was synthesized from ethyl 2-(2-(1-hydroxy-1-(pyrimidin-4-yl)ethyl)furo[3,2-b]pyridin-5-yl)acetate following essentially the procedure of Examples 31 (b) (181 mg, yield: 75%). LCMSA027: 299.9 [M+H]⁺; Rt=0.75 min

### h) N—((S)-(2,4-dimethylphenyl)(phenyl)methyl)-2-(2-(1-hydroxy-1-(pyrimidin-4-yl)ethyl)furo[3,2-b]pyridin-5-yl)acetamide

The title compound was synthesized from 2-(2-(1-hydroxy-1-(pyrimidin-4-yl)ethyl)furo[3,2-b]pyridin-5-yl)acetic acid and (S)-(2,4-dimethylphenyl)(phenyl)methanamine following essentially the procedure of Examples 2 (b) (21 mg, yield: 7.2%). LC-MS038: 493.2[M+H]⁺; Rt=1.67 min. ¹H NMR (CDCl₃, 400 MHz): δ 9.09 (d, J=1.2 Hz, 1H), 8.86 (d, J=5.6 Hz, 1H), 8.18 (d, J=8.4 Hz, 1H), 7.97-7.96 (m, 1H), 7.52 (d, J=8.4 Hz, 1H), 7.31-7.22 (m, 6H), 7.10-7.00 (m, 5H), 6.33 (s, 1H), 2.29 (s, 3H), 2.21 (s, 3H), 2.04 (s, 3H).

### Example 95

### N—((S)-(2,4-dimethylphenyl)(phenyl)methyl)-2-(2-(1-hydroxy-1-(2-methylpyridin-4-yl)ethyl)furo[2,3-b]pyridin-5-yl)acetamide

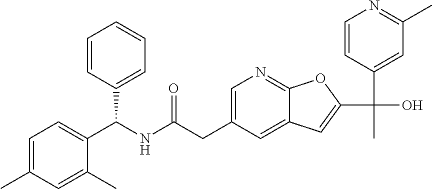

### a) diethyl 2-(6-methoxypyridin-3-yl)malonate

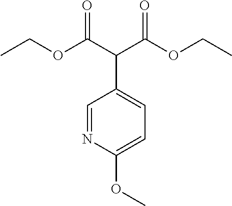 [SMILES: CCOC(=O)C(C(=O)OCC)c1ccc(OC)nc1]

NaH (3.6 g, 90 mmol) was added portion-wise to a solution of 5-bromo-2-methoxypyridine (5.6 g, 30 mmol) in dioxane (100 mL) under N₂ protection, then CuBr (8.6 g, 60 mmol), and then followed by addition of diethyl malonate (9.6 g 60 mmol). Then the mixture was warmed up to 100° C. and stirred overnight. The mixture was then filtered and the filtered residue was washed with EtOAc (3*40 mL). The combined filtrates were concentrated under reduced pressure and the resulting residue was purified by silica-gel-column (petroleum ether:EtOAc=5:1) to get diethyl 2-(6-methoxypyridin-3-yOmalonate (9.2 g), which was carried through without further purification. LC-MS (036): 268.0 [M+H]⁺; Rt: 1.63 min.

### b) 2-(6-methoxypyridin-3-yl)acetic acid

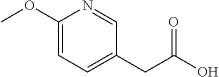

A solution of NaOH (8.97 g, 74.2 mmol) in water (50 mL) was added to a solution of diethyl 2-(6-methoxypyridin-3-yl)malonate (9.2 g, 37.2 mmol) in water (50 mL). Then the mixture was stirred at rt for 2 h. The reaction mixture was then acidified to pH ˜1 by adding 6 M HCl. The resulting mixture was refluxed overnight. The pH of the reaction mixture was then adjusted pH ˜6-7 by adding sat. NaHCO₃. The resulting mixture was extracted with EtOAc (3*60 mL). The combined extracts were concentrated under reduced pressure to give 2-(6-methoxypyridin-3-yl)acetic acid (1.48 g, 23.8%) as a colorless liquid, which was carried through without further purification. LC-MS (036): 168.1 [M+H]⁺; Rt: 1.16 min.

### c) ethyl 2-(6-hydroxypyridin-3-yl)acetate

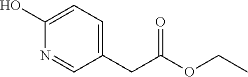

The title compound was synthesized from 2-(6-methoxypyridin-3-yl)acetic acid and ethanol following essentially the procedure of Examples 9 (a) (642 mg, 40.1%). LC-MS (044): 182.1 [M+H]⁺; Rt: 1.21 min; Purity: 51.5% (254 nm).

### d) ethyl 2-(6-hydroxy-5-iodopyridin-3-yl)acetate

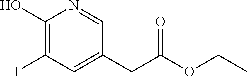

NIS (887 mg, 3.90 mmol) was added to a solution of ethyl 2-(6-hydroxypyridin-3-yl)acetate (642 mg, 3.54 mmol) in CH₃CN (50 mL) under N₂, then the mixture was heated to reflux for 2 h. Then the mixture was allowed to cool to rt and stirred overnight. The mixture was then extracted with EtOAc (80 mL). The combined extracts were washed with H₂O (3×40 mL), dried with sodium sulfate, and concentrated under reduced pressure. The resulting residue was purified by silica-gel-column (petroleum ether:EtOAc=2:1) to get ethyl 2-(6-hydroxy-5-iodopyridin-3-yl)acetate (900 mg, yield 82.4%) as a light yellow solid. LC-MS (036): 308.0 [M+H]⁺; Rt: 1.31 min.

### e) ethyl 2-(2-(1-hydroxy-1-(2-methylpyridin-4-yl)ethyl)furo[2,3-b]pyridin-5-yl)acetate

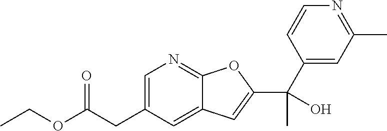 [SMILES: CCOC(=O)Cc1cnc2oc(C(C)(O)c3ccnc(C)c3)cc2c1]

The title compound was synthesized from ethyl 2-(6-hydroxy-5-iodopyridin-3-yl)acetate following essentially the procedure of Examples 94 (f) (160 mg, yield 84.6%) except using Pd(dppf)Cl₂ instead of Pd(Ph₃P)₂Cl₂. LC-MS (039): 162.3 [M+H]⁺; Rt: 1.25 min.

### f) 2-(2-(1-hydroxy-1-(2-methylpyridin-4-yl)ethyl)furo[2,3-b]pyridin-5-yl)acetic acid

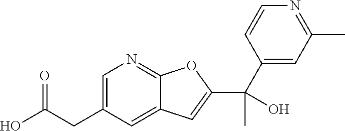 [SMILES: Cc1cc(C(C)(O)c2cc3cc(CC(=O)O)cnc3o2)ccn1]

The title compound was synthesized from ethyl 2-(2-(1-hydroxy-1-(2-methylpyridin-4-yl)ethyl)furo[2,3-b]pyridin-5-yl)acetate following essentially the procedure of Examples 31 (b). LC-MS (039): 313.2 [M+H]⁺; Rt: 1.08 min.

### g) N—((S)-(2,4-dimethylphenyl)(phenyl)methyl)-2-(2-(1-hydroxy-1-(2-methylpyridin-4-yl)ethyl)furo[2, 3-1)]pyridin-5-yl)acetamide

The title compound was synthesized from 2-(2-(1-hydroxy-1-(2-methylpyridin-4-yl)ethyl)furo[2,3-b]pyridin-5-yl)acetic acid and (S)-(2,4-dimethylphenyl)(phenyl)methanamine following essentially the procedure of Examples 2 (b) (19 mg, 19.5%). LC-MS (039): 506.3 [M+H]⁺; Rt: 1.66 min. ¹H-NMR (MeOD, 400 MHz): δ 9.01 (d, J=7.6 Hz, 1H), 8.64 (d, J=6.4 Hz, 1H), 8.18 (d, J=2.0 Hz, 1H), 8.10 (s, 1H), 8.03 (d, J=2.4 Hz, 1H), 7.33-7.26 (m, 3H), 7.17 (d, J=7.2 Hz, 2H), 7.01-6.95 (m, 4H), 6.30 (d, J=8.4 Hz, 1H), 3.73 (s, 3H), 2.78 (s, 3H), 2.29 (s, 3H), 2.19 (s, 3H), 2.04 (s, 3H)

### Example 96

### 1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid

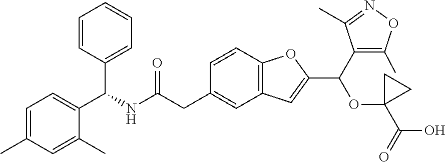 [SMILES: Cc1ccc([C@@H](NC(=O)Cc2ccc3oc(C(OC4(C(=O)O)CC4)c4c(C)noc4C)cc3c2)c2ccccc2)c(C)c1]

### a) ethyl 1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylate

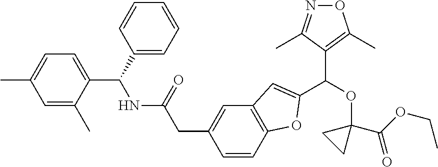 [SMILES: CCOC(=O)C1(OC(c2cc3cc(CC(=O)N[C@@H](c4ccccc4)c4ccc(C)cc4C)ccc3o2)c2c(C)noc2C)CC1]

The title compound was synthesized from 2-(2-((3,5-dimethylisoxazol-4-yl)(hydroxy)methyl)benzofuran-5-yl)-N—((S)-(2,4-dimethylphenyl)(phenyl)methyl)acetamide and ethyl 1-hydroxycyclopropanecarboxylate following essentially the procedure of Examples 63 (b) (40 mg, yield: 46.5%). LC-MSA036: 606.7 [M+H]⁺; Rt=1.906 min.

### b) 1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid

The title compound was synthesized from ethyl 1((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylate following essentially the procedure of Examples 31 (b) (15 mg, yield 30.6%). LC-MS (038): 579.7 [M+H]⁺; Rₜ=1.787 min. ¹H NMR (DMSO-d6, 500 MHz): δ 8.88 (d, J=8.4 Hz, 1H), 7.46 (t, J=8.8 Hz, 2H), 7.30 (t, J=7.2 Hz, 2H), 7.25-7.15 (m, 4H), 7.00-6.93 (m, 3H), 6.87 (s, 1H), 6.18 (d, J=8.0 Hz, 1H), 5.96 (s, 1H), 3.58 (s, 2H), 2.40 (s, 3H), 2.22 (s, 3H), 2.14-2.13 (m, 6H), 1.18-1.10 (m, 4H).

### Example 97

### 2-(2-((3-aminoazetidin-1-yl)(3,5-dimethylisoxazol-4-yl)methyl)benzofuran-5-yl)-N-((4-hydroxy-2-methylphenyl)(phenyl)methyl)acetamide

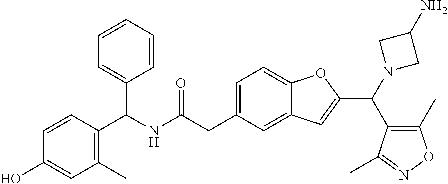 [SMILES: Cc1cc(O)ccc1C(NC(=O)Cc1ccc2oc(C(c3c(C)noc3C)N3CC(N)C3)cc2c1)c1ccccc1]

### a) 4-hydroxy-2-methylbenzonitrile

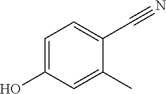

Decanethiol (261 mg, 1.5 mmol) and t-BuOK (168 mg, 1.5 mmol) were added to a solution of 4-methoxy-2-methylbenzonitrile (147 mg, 1 mmol) in DMF (5 mL). The reaction mixture was stirred at 110° C. for 3 h. The mixture was then diluted with water (30 mL) and extracted with EtOAc (10 mL×3). The extracts were washed with brine (10 mL×3), dried over Na₂SO₄, and concentrated under reduced pressure. The resulting residue was purified by silica gel column with (petroleum ether:EtOAc=10:1) to provide 4-hydroxy-2-methylbenzonitrile (70 mg, yield: 52.6%). LC-MS (011): 134.70 [M+H]⁺; Rt: 1.44 min, Purity: 80% (254 nm).

### b) 4-(benzyloxy)-2-methylbenzonitrile

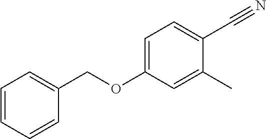 [SMILES: Cc1cc(OCc2ccccc2)ccc1C#N]

BnCl (806.5 mg, 4.69 mmol) and K₂CO₃ (1175.7 mg, 8.52 mmol) were added to a solution of 4-hydroxy-2-methylbenzonitrile (567 mg, 4.26 mmol) in acetonitrile (10 mL). The resulting mixture was stirred overnight at rt. The mixture was then diluted with water (30 mL) and extracted with EtOAc. The combined extracts were washed with brine (10 mL×3), dried over Na₂SO₄, and concentrated under reduced pressure to provide crude 4-(benzyloxy)-2-methylbenzonitrile (500 mg, yield: 52.6%), which used in the next step without further purification. LC-MS (026): 224.7 [M+H]⁺; Rt=1.63 min, purity 58%

### c) (4-(benzyloxy)-2-methylphenyl)(phenyl)methanamine

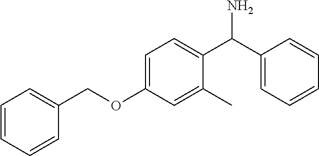 [SMILES: Cc1cc(OCc2ccccc2)ccc1C(N)c1ccccc1]

This compound was synthesized from 4-(benzyloxy)-2-methylbenzonitrile and phenylmagnesium bromide essentially as described in example 1 (e) (400 mg, yield: 37%.). LC-MS (010): 288.7 [M−NH2]⁺; Rt=1.593 min, purity: 100% 214.

### d) N-((4-(benzyloxy)-2-methylphenyl)(phenyl)methyl)-2-(2-((3,5-dimethylisoxazol-4-yl)(hydroxy)methyl)benzofuran-5-yl)acetamide

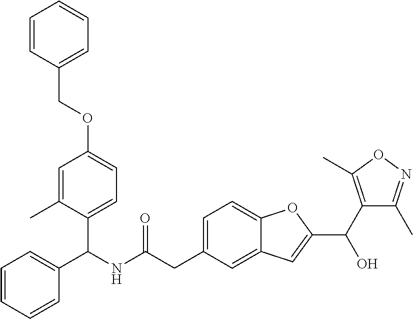 [SMILES: Cc1cc(OCc2ccccc2)ccc1C(NC(=O)Cc1ccc2oc(C(O)c3c(C)noc3C)cc2c1)c1ccccc1]

HOBt (62.3 mg, 0.462 mmol), EDCI (88.2 mg, 0.462 mmol), DIPEA (59.6 mg, 0.462 mmol) and 4-(benzyloxy)-2-methylphenyl)(phenyl)methanamine (70 mg, 0.231 mmol) were added to a solution of 2-(24(3,5-dimethylisoxazol-4-yl)(hydroxy)methyl)benzofuran-5-yl)acetic acid (69.5 mg, 0.231 mmol) in CH₂Cl₂ (5 mL). The resulting mixture was stirred at rt overnight, then additional CH₂Cl₂ (5 mL) was added to the mixture. The mixture was then washed with water (10 mL×3), brine (10 mL×3), dried over Na₂SO₄, and concentrated under reduced pressure to yield a residue that was used directly in the next step without further purification. LC-MS (024): 569.7 [M−OH]⁺; Rt=1.538 min, purity 89%.

### e) tert-butyl (14(5-(2-(((4-(benzyloxy)-2-methylphenyl)(phenyl)methyl)amino)-2-oxoethyl)benzofuran-2-yl)(3,5-dimethylisoxazol-4-yl)methyl)azetidin-3-yl)carbamate

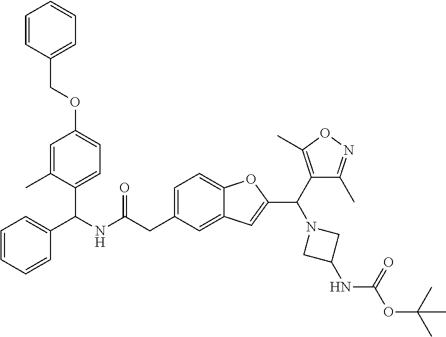 [SMILES: Cc1cc(OCc2ccccc2)ccc1C(NC(=O)Cc1ccc2oc(C(c3c(C)noc3C)N3CC(NC(=O)OC(C)(C)C)C3)cc2c1)c1ccccc1]

The title compound was synthesized from N-((4-(benzyloxy)-2-methylphenyl)(phenyl)methyl)-2-(2-((3,5-dimethylisoxazol-4-yl)(hydroxy)methyl)benzofuran-5-yl)acetamide following essentially the procedure of Example 39 steps (d) and (e) (100 mg, yield 68%). LC-MSA036: 741.70 [M+H]⁺; Rt=1.643 min

### f) tert-butyl (1-((3,5-dimethylisoxazol-4-yl)(5-(2-(((4-hydroxy-2-methylphenyl)(phenyl)methyl)amino)-2-oxoethyl)benzofuran-2-yl)methyl)azetidin-3-yl)carbamate

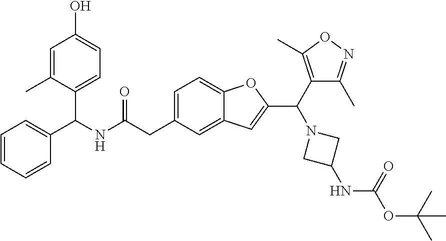 [SMILES: Cc1cc(O)ccc1C(NC(=O)Cc1ccc2oc(C(c3c(C)noc3C)N3CC(NC(=O)OC(C)(C)C)C3)cc2c1)c1ccccc1]

The mixture of tert-butyl (1-((5-(2-(((4-(benzyloxy)-2-methylphenyl)(phenyl)methyl)amino)-2-oxoethyl)benzofuran-2-yl)(3,5-dimethylisoxazol-4-yl)methyl)azetidin-3-yl)carbamate (50 mg, 0.068 mmol) and 10% Pd/C (6 mg) in EtOAc (8 mL) was stirred under hydrogen atmosphere (latm) for 5 h at rt. Then the catalyst was filtered off and the filtrate was concentrated under reduced pressure. The resulting residue was purified by Pre-HPLC to obtain tert-butyl (1-((3,5-dimethylisoxazol-4-yl)(5-(2-(((4-hydroxy-2-methylphenyl)(phenyl)methyl)amino)-2-oxoethyl)benzofuran-2-yl)methyl)azetidin-3-yl)carbamate (30 mg, yield: 68.2%.). LCMSA027: 651.70 [M+H]⁺; Rt=1.045 min.

### g) 2-(2-((3-amino azetidin-1-yl)(3,5-dimethylisoxazol-4-yl)methyl)benzofuran-5-yl)-N-((4-hydroxy-2-methylphenyl)(phenyl)methyl)acetamide

The title compound was synthesized from tert-butyl (1-((3,5-dimethylisoxazol-4-yl)(5-(2-(((4-hydroxy-2-methylphenyl)(phenyl)methyl)amino)-2-oxoethyl)benzofuran-2-yl)methyl)azetidin-3-yl)carbamate following essentially the procedure of Example 39 (f) (8 mg, yield 47%). LCMSA044: 551.70 [M+H]⁺; Rt=1.51 min. ¹H NMR (DMSO, 400 MHz): δ 8.80 (d, J=8.4 Hz, 1H), 8.21 (s, 3H), 7.46 (s, 1H), 741 (d, J=8.4 Hz, 1H), 7.31-7.15 (m, 7H), 6.82 (d, J=8.4 Hz, 1H), 6.77 (s, 1H), 6.57 (s, 1H), 6.51 (d, J=6.4 Hz, 1H), 6.12 (d, J=8.8 Hz, 1H), 4.74 (s, 1H), 3.62-3.17 (m, 7H), 2.50 (s, 3H), 2.21 (s, 3H), 2.11 (s, 3H).

### Example 98

### 2-(2-((3-aminoazetidin-1-yl)(3,5-dimethylisoxazol-4-yl)methyl)benzofuran-5-yl)-N-((4-methoxy-2-methylphenyl)(phenyl)methyl)acetamide

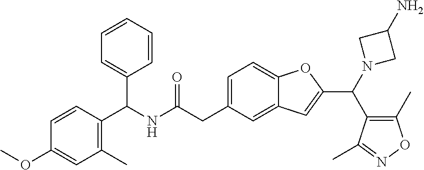

### a) tert-butyl (1-((3,5-dimethylisoxazol-4-yl)(5-(2-(((4-methoxy-2-methylphenyl)(phenyl)methyl)amino)-2-oxoethyl)benzofuran-2-yl)methyl)azetidin-3-yl)carbamate

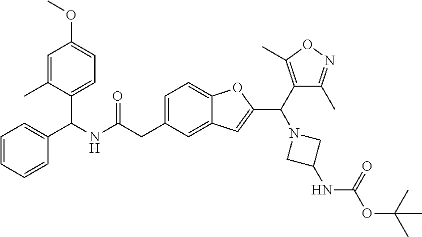 [SMILES: COc1ccc(C(NC(=O)Cc2ccc3oc(C(c4c(C)noc4C)N4CC(NC(=O)OC(C)(C)C)C4)cc3c2)c2ccccc2)c(C)c1]

The title compound was synthesized from tert-butyl (1-((3,5-dimethylisoxazol-4-yl)(5-(2-(((4-hydroxy-2-methylphenyl)(phenyl)methyl)amino)-2-oxoethyl)benzofuran-2-yl)methyl)azetidin-3-yl)carbamate following essentially the procedure of Example 19 (a) (20 mg, yield 65.6%). LCMSA027: 665.70 [M+H]⁺; Rt=1.125 min.

### b) 2-(2-((3-aminoazetidin-1-yl)(3,5-dimethylisoxazol-4-yl)methyl)benzofuran-5-yl)-N-((4-methoxy-2-methylphenyl)(phenyl)methyl)acetamide

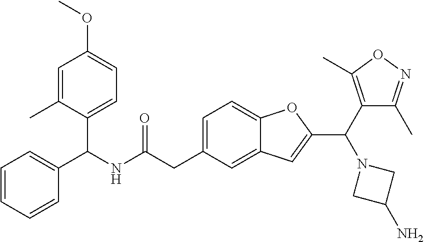 [SMILES: COc1ccc(C(NC(=O)Cc2ccc3oc(C(c4c(C)noc4C)N4CC(N)C4)cc3c2)c2ccccc2)c(C)c1]

The title compound was synthesized from tert-butyl (1-((3,5-dimethylisoxazol-4-yl)(5-(2-(((4-methoxy-2-methylphenyl)(phenyl)methyl)amino)-2-oxoethyl)benzofuran-2-yl)methyl)azetidin-3-yl)carbamate following essentially the procedure of Example 39 (f) (8 mg, yield 47%). LCMSA044: 565.70 [M+H]⁺; Rt=1.639 min; purity: 100% (254 nm). ¹H NMR (DMSO, 400 MHz): δ 8.80 (d, J=8.4 Hz, 1H), 8.21 (s, 3H), 7.46 (s, 1H), 741 (d, J=8.4 Hz, 1H), 7.31-7.15 (m, 7H), 6.82 (d, J=8.4 Hz, 1H), 6.77 (s, 1H), 6.57 (s, 1H), 6.51 (d, J=6.4 Hz, 1H), 6.12 (d, J=8.8 Hz, 1H), 4.74 (s, 1H), 3.62-3.17 (m, 7H), 2.50 (s, 3H), 2.21 (s, 3H), 2.11 (s, 3H).

### Example 99

### 2-(2-((2-amino-2-methylpropoxy)(3,5-dimethylisoxazol-4-yl)methyl)benzofuran-6-yl)-N-((4-hydroxy-2-methylphenyl)(phenyl)methyl)acetamide

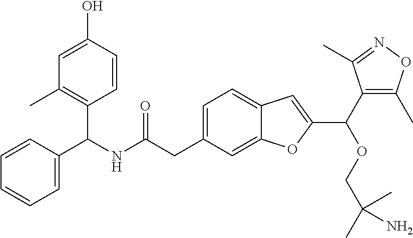 [SMILES: Cc1cc(O)ccc1C(NC(=O)Cc1ccc2cc(C(OCC(C)(C)N)c3c(C)noc3C)oc2c1)c1ccccc1]

The title compound was synthesized from 2-(2-((3,5-dimethylisoxazol-4-yl)(hydroxy)methyl)benzofuran-6-yl)-N-((4-hydroxy-2-methylphenyl)(phenyl)methyl)acetamide and 2-amino-2-methylpropan-1-ol following essentially the procedure of Example 63 (b) (5 mg, yield 15.6%). LCMSA044: 479.70 [M−88]⁺; Rt=1.24 min. ¹H NMR (DMSO, 400 MHz): δ 9.24 (s, 1H), 8.81 (d, J=8.0 Hz, 1H), 7.85 (s, 2H), 7.48 (m, 2H), 7.31-7.16 (m, 6H), 6.98 (d, J=7.6 Hz, 1H), 6.82 (d, J=8.4 Hz, 1H), 6.57 (s, 1H), 6.51 (d, J=8.8 Hz, 1H), 6.12 (d, J=8.4 Hz, 1H), 5.75 (s, 1H), 3.59 (s, 2H), 3.50-3.46 (m, 2H), 2.41 (s, 3H), 2.18 (s, 3H), 2.11 (s, 3H), 1.26 (s, 3H), 1.22 (s, 3H).

### Examples 100 and 101

### 1-((R)-(3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid and 1-((S)-(3,5-dimethylisoxazol-4-yl)(5-(2-((S)-(2,4-dimethylphenyl)(phenyl)methyl)amino)-2-oxoethyl)benzofuran-2-yl)methoxy)cyclopropanecarboxylic acid

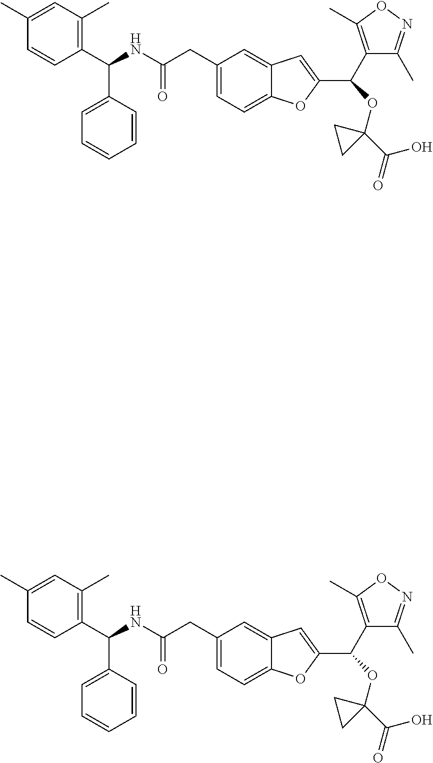 [SMILES: Cc1ccc([C@@H](NC(=O)Cc2ccc3oc([C@H](OC4(C(=O)O)CC4)c4c(C)noc4C)cc3c2)c2ccccc2)c(C)c1 ; Cc1ccc([C@@H](NC(=O)Cc2ccc3oc([C@@H](OC4(C(=O)O)CC4)c4c(C)noc4C)cc3c2)c2ccccc2)c(C)c1]

### a) 2-(2-((S)-(3,5-dimethylisoxazol-4-yl)(hydroxy)methyl)benzofuran-5-yl)-N—((S)-(2,4-dimethylphenyl)(phenyl)methyl)acetamide

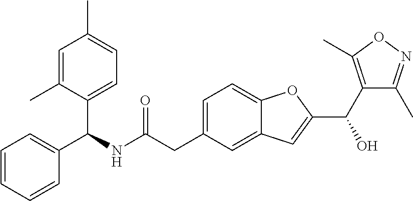 [SMILES: Cc1ccc([C@@H](NC(=O)Cc2ccc3oc([C@@H](O)c4c(C)noc4C)cc3c2)c2ccccc2)c(C)c1]

This compound was synthesized from (S)-(2,4-dimethylphenyl)(phenyl)methanamine and (S)-methyl 2-(2-((3,5-dimethylisoxazol-4-yl)(hydroxy)methyl)benzofuran-5-yl)acetate essentially as described in example 2(b) (85 g, yield: 86.7%) as a white solid.

### b) ethyl 1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylate

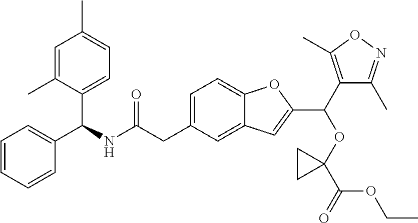 [SMILES: CCOC(=O)C1(OC(c2cc3cc(CC(=O)N[C@@H](c4ccccc4)c4ccc(C)cc4C)ccc3o2)c2c(C)noc2C)CC1]

The title compound was synthesized from 2-(2-((S)-(3,5-dimethylisoxazol-4-yl)(hydroxy)methyl)benzofuran-5-yl)-N—((S)-(2,4-dimethylphenyl)(phenyl)methyl)acetamide following essentially the procedure of Example 63 (b) (420 mg, yield: 34.6%). LC-MS (036): 607.7 [M+H]⁺; Rt: 1.93 min

### c) ethyl 1-((R)-(3,5-dimethylisoxazol-4-yl)(5-(2-((S)-(2,4-dimethylphenyl)(phenyl)methyl)amino)-2-oxoethyl)benzofuran-2-yl)methoxy)cyclopropanecarboxylate and ethyl 1-((S)-(3,5-dimethylisoxazol-4-yl)(5-(2-((S)-(2,4-dimethylphenyl)(phenyl)methyl)amino)-2-oxoethyl)benzofuran-2-yl)methoxy)cyclopropanecarboxylate ethyl 1-((3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylate (420 mg) was resolved using the following method:

Instrument: Thar SFC Prep 80 (Thar Technologies, Waters); Column: ChiralPak AS-H, 30 mm I.D.×250 mm Length, 5 μm (Daicel Chemical Industries Co., Ltd); Column Temperature: 35; Mobile Phase: CO₂/IPA/DEA=60/40/0.1; Flow rate: 70 g/min; Back Pressure: 100 Bar; Wavelength: 214 nm; Cycle time: 7.4 min; Injection Volume: 2.0 mL; Load per injection: 38.2 mg; Feed solution: 420 mg dissolved in 22 mL MeOH

Said resolution yielded 2 diastereomers:
Peak 1, 150 mg, RT: 4.2 min; >99% de
Peak 2, 150 mg, RT: 5.8 min; >99% de

### d) 1-((R)-(3,5-dimethylisoxazol-4-yl)(5-(2-(((S)-(2,4-dimethylphenyl)(phenyl)methyl)amino)-2-oxoethyl)benzofuran-2-yl)methoxy)cyclopropanecarboxylic acid and 1-((S)-(3,5-dimethylisoxazol-4-yl)(5-(2-((S)-(2,4-dimethylphenyl)(phenyl)methyl)amino)-2-oxoethyl)benzofuran-2-yl)methoxy)cyclopropanecarboxylic acid

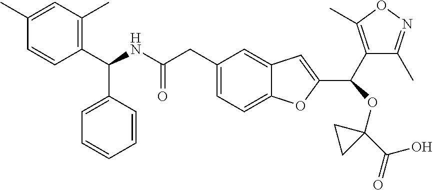 [SMILES: Cc1ccc([C@@H](NC(=O)Cc2ccc3oc([C@H](OC4(C(=O)O)CC4)c4c(C)noc4C)cc3c2)c2ccccc2)c(C)c1]

Peak 1 (150 mg, 0.25 mmol) was dissolved in 5 mL of THF and 3 mL of water. LiOH (21 mg, 0.5 mmol) was added and the mixture was stirred at rt for 3 h. The mixture was then acidified with 1% HCl to pH 6-7, and extracted with ethyl acetate (50 mL×3). The combined extracts were washed with brine (20 mL×3), dried over Na₂SO₄, and concentrated under reduced pressure to yield peak 1-acid (80 mg, yield: 53%). LC-MS (044): 579.3 [M+H]⁺; Rt: 1.51 min. ¹H NMR (CDCL3, 400 MHz): δ 7.34-7.13 (m, 6H), 6.97-6.54 (m, 6H), 6.01-5.81 (m, 3H), 3.60 (s, 2H), 2.34 (s, 3H), 2.19 (s, 3H), 2.14 (s, 3H), 2.10 (s, 3H), 1.28-1.11 (m, 4H).

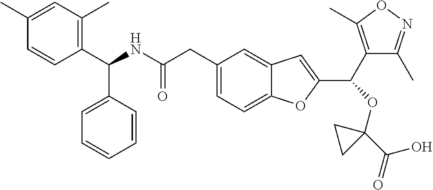 [SMILES: Cc1ccc([C@@H](NC(=O)Cc2ccc3oc([C@@H](OC4(C(=O)O)CC4)c4c(C)noc4C)cc3c2)c2ccccc2)c(C)c1]

Peak 2 (150 mg, 0.25 mmol) was dissolved in 5 mL of THF and 3 mL of water. LiOH (21 mg, 0.5 mmol) was added and the mixture was stirred at rt for 3 h. The mixture was then acidified with 1% HCl to pH ˜6-7, and extracted with ethyl acetate (50 mL×3). The combined extracts were washed with brine (20 mL×3), dried over Na₂SO₄, and concentrated under reduced pressure to yield peak 2-acid (48 mg, yield: 33.2%). LC-MS (044): 579.3 [M+H]⁺; Rt: 1.51 min. ¹H NMR (CDCL3, 400 MHz): δ 7.23-7.11 (m, 6H), 7.09-6.75 (m, 6H), 6.30-5.90 (m, 3H), 3.19 (s, 2H), 2.14 (s, 3H), 2.12 (s, 3H), 2.04 (s, 3H), 1.97 (s, 3H), 1.20-0.77 (m, 4H).

### Examples 102 and 103

### 2-(2-((R)-(3-aminoazetidin-1-yl)(3,5-dimethylisoxazol-4-yl)methyl)benzofuran-5-yl)-N—((S)-(2,4-dimethylphenyl)(phenyl)methyl)acetamide and 2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide

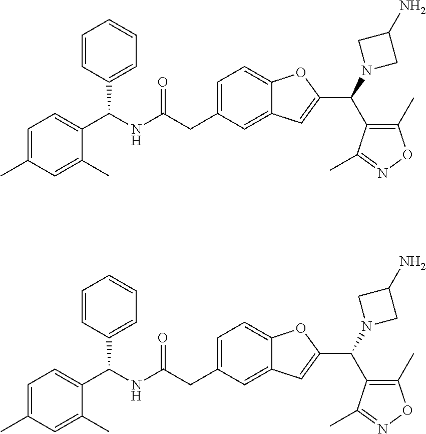

### a) tert-butyl (1-((3,5-dimethylisoxazol-4-yl)(5-(2-(((S)-(2,4-dimethylphenyl)(phenyl)methyl)amino)-2-oxoethyl)benzofuran-2-yl)methyl)azetidin-3-yl)carbamate

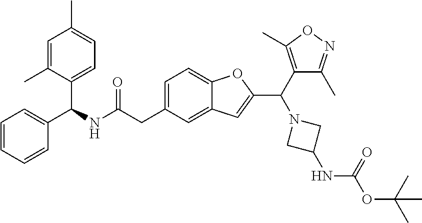 [SMILES: Cc1ccc([C@@H](NC(=O)Cc2ccc3oc(C(c4c(C)noc4C)N4CC(NC(=O)OC(C)(C)C)C4)cc3c2)c2ccccc2)c(C)c1]

This compound was synthesized from (S)-(2,4-dimethylphenyl)(phenyl)methanamine and 2-(2-((3,5-dimethylisoxazol-4-yl)(hydroxy)methyl)benzofuran-5-yl)acetic acid essentially as described in example 39 steps (c), (d) and (e) (5.6 g, yield 52%). LC-MSA036: 649.3[M+H]⁺; Rt=1.603 min, purity 89% (254 nm).

### b) tert-butyl (1-((R)-(3,5-dimethylisoxazol-4-yl)(5-(2-((S)-(2,4-dimethylphenyl)(phenyl)methyl)amino)-2-oxoethyl)benzofuran-2-yl)methyl)azetidin-3-yl)carbamate and tert-butyl (1-((S)-(3,5-dimethylisoxazol-4-yl)(5-(2-(((S)-(2,4-dimethylphenyl)(phenyl)methyl)amino)-2-oxoethyl)benzofuran-2-yl)methyl)azetidin-3-yl)carbamate

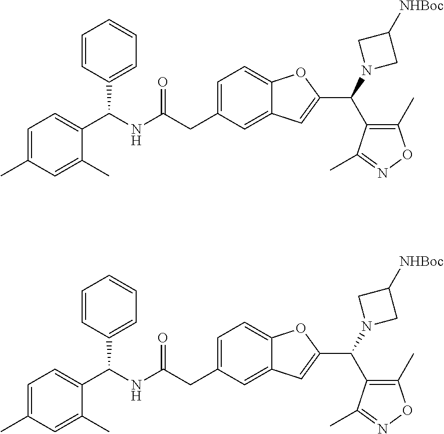

tert-butyl (1-((3,5-dimethylisoxazol-4-yl)(5-(2-(((S)-(2,4-dimethylphenyl)(phenyl)methyl)amino)-2-oxoethyl)benzofuran-2-yl)methyl)azetidin-3-yl)carbamate (5.6 g) was resolved using the following method: Instrument: Thar SFC Prep 200 (Thar Technologies, Waters); Column: RegisCell, 50 mm I.D.×250 mm Length, 5 μm (Regis Technologies); Column Temperature: 35° C.; Mobile Phase: CO₂/MeOH/DEA=60/40/0.1; Flow rate: 150 g/min; Back Pressure: 100 Bar; Wavelength: 214 nm Cycle time: 8.0 min; Injection Volume: 2.0 mL; Load per injection: 273 mg.

Said resolution yielded 2 diastereomers:
Peak 1, 1.59 g, RT: 9.8 min, 95% ee; 98% purity;
Peak 2, 1.68 g, RT: 12.0 min, 99% ee; 99% purity

### c) 2-(2-((R)-(3-aminoazetidin-1-yl)(3,5-dimethylisoxazol-4-yl)methyl)benzofuran-5-yl)-N—((S)-(2,4-dimethylphenyl)(phenyl)methyl)acetamide and 2-(2-((S)-(3-aminoazetidin-1-yl)(3,5-dimethylisoxazol-4-yl)methyl)benzofuran-5-yl)-N—((S)-(2,4-dimethylphenyl)(phenyl)methyl)acetamide

The title compounds were synthesized from tert-butyl (1-((R)-(3,5-dimethylisoxazol-4-yl)(5-(2-(((S)-(2,4-dimethylphenyl)(phenyl)methyl)amino)-2-oxoethyl)benzofuran-2-yl)methyl)azetidin-3-yl)carbamate and tert-butyl (1-((S)-(3,5-dimethylisoxazol-4-yl)(5-(2-(((S)-(2,4-dimethylphenyl)(phenyl)methyl)amino)-2-oxoethyl)benzofuran-2-yl)methyl)azetidin-3-yl)carbamate essentially as described in example 39 steps (f)
product from peak 1: 1.18 g, 100% yield; LC-MSA036: Fragment at 477.2; Rt=1.330 min. ¹H NMR (MeOD, 400 MHz): δ 7.50 (s, 1H), 7.36 (d, J=8.0 Hz, 1H), 7.31-7.23 (m, 4H), 7.15 (d, J=7.2 Hz, 2H), 6.99-6.92 (m, 3H), 6.70 (s, 1H), 6.30 (s, 1H), 4.80-4.78 (m, 1H), 3.98-3.92 (m, 1H), 3.75-3.61 (m, 4H), 3.39-3.31 (m, 2H), 2.56 (s, 3H), 2.32 (s, 3H), 2.28 (s, 3H), 2.17 (s, 3).
product from peak 2 yielded 1.2 g, 100% yield; LC-MSA036: 571.2 [M+Na]⁺; Rt=1.330 min. ¹H NMR (MeOD, 400 MHz): δ 7.50 (s, 1H), 7.36 (d, J=8.0 Hz, 1H), 7.31-7.23 (m, 4H), 7.15 (d, J=7.2 Hz, 2H), 6.99-6.92 (m, 3H), 6.70 (s, 1H), 6.30 (s, 1H), 4.80-4.78 (m, 1H), 3.98-3.92 (m, 1H), 3.75-3.61 (m, 4H), 3.39-3.31 (m, 2H), 2.56 (s, 3H), 2.32 (s, 3H), 2.28 (s, 3H), 2.17 (s, 3).

### Example 104

### 2-(2((3,5-dimethylisoxazol-4-yl)(3-(methylsulfonyl)azetidin-1-yl)methyl)furo[3,2-b]pyridin-5-yl)-N—((S)-(2,4-dimethylphenyl)(phenyl)methyl)acetamide

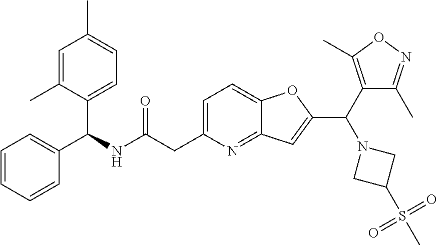 [SMILES: Cc1ccc([C@@H](NC(=O)Cc2ccc3oc(C(c4c(C)noc4C)N4CC(S(C)(=O)=O)C4)cc3n2)c2ccccc2)c(C)c1]

### a) 2-(2-(chloro(3,5-dimethylisoxazol-4-yl)methyl)furo[3,2-b]pyridin-5-yl)-N—((S)-(2,4-dimethylphenyl)(phenyl)methyl)acetamide

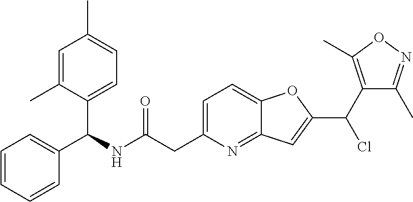 [SMILES: Cc1ccc([C@@H](NC(=O)Cc2ccc3oc(C(Cl)c4c(C)noc4C)cc3n2)c2ccccc2)c(C)c1]

This compound was synthesized from 2-(2-((3,5-dimethylisoxazol-4-yl)(hydroxy)methyl)furo[3,2-b]pyridin-5-yl)-N—((S)-(2,4-dimethylphenyl)(phenyl)methyl)acetamide essentially as described in example 28 (a) (64 mg of crude product). LCMSA044: 538[M−Cl+OMe⁺ H]⁺; Rt: 1.86 min

### b) 2-(2-((3,5-dimethylisoxazol-4-yl)(3-(methylsulfonyl)azetidin-1-yl)methyl)furo[3,2-b]pyridin-5-yl)-N—((S)-(2,4-dimethylphenyl)(phenyl)methyl)acetamide

This compound was synthesized from 2-(2-(chloro(3,5-dimethylisoxazol-4-yl)methyl)furo[3,2-b]pyridin-5-yl)-N—((S)-(2,4-dimethylphenyl)(phenyl)methyl)acetamide and 3-(methylsulfonyl)azetidine essentially as described in example 39 (e) (18 mg, yield: 40%). LCMSA020: 613.2 [M+H]⁺; Rt: 1.83 min. ¹H NMR (MeOD, 400 MHz): δ 7.88 (br, 1H), 7.39-7.20 (m, 6H), 7.06-6.96 (m, 4H), 6.32 (s, 1H), 4.90 (s, 1H), 4.22-4.15 (m, 1H), 3.98-3.93 (m, 1H), 3.81-3.61 (m, 4H), 3.29-3.22 (m, 1H), 3.00 (s, 3H), 2.57 (s, 3H), 2.32 (s, 3H), 2.29 (s, 3H), 2.21 (s, 3H).

### Example 105

### 1-(1-(4-fluorophenyl)-6-methyl-3,4-dihydroisoquinolin-2(1H)-yl)-2-(2-(1-hydroxy-1-(pyridin-4-yl)ethyl)benzofuran-5-yl)ethanone

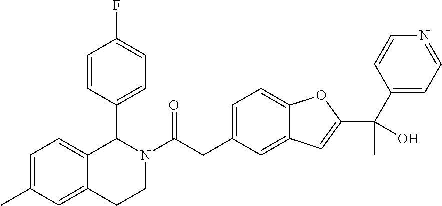 [SMILES: Cc1ccc2c(c1)CCN(C(=O)Cc1ccc3oc(C(C)(O)c4ccncc4)cc3c1)C2c1ccc(F)cc1]

### a) 1-(1-(4-fluorophenyl)-6-methyl-3,4-dihydroisoquinolin-2(1H)-yl)-2-(2-isonicotinoylbenzofuran-5-yl)ethanone

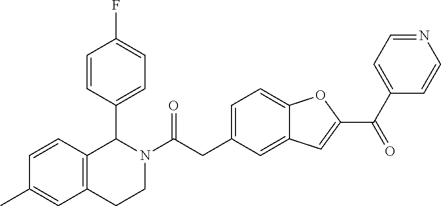 [SMILES: Cc1ccc2c(c1)CCN(C(=O)Cc1ccc3oc(C(=O)c4ccncc4)cc3c1)C2c1ccc(F)cc1]

The title compound was synthesized from 1-(4-fluorophenyl)-6-methyl-1,2,3,4-tetrahydroisoquinoline and 2-(2-isonicotinoylbenzofuran-5-yl)acetic acid following essentially the procedures of Example 2 (b) (560 mg, yield: 125%). LC-MS (044): 505.2 [M+H]⁺; Rt: 1.98 min.

### b) 1-(1-(4-fluorophenyl)-6-methyl-3,4-dihydroisoquinolin-2(1H)-yl)-2-(2-(1-hydroxy-1-(pyridin-4-yl)ethyl)benzofuran-5-yl)ethanone

The title compound was synthesized from 1-(1-(4-fluorophenyl)-6-methyl-3,4-dihydroisoquinolin-2(1H)-yl)-2-(2-isonicotinoylbenzofuran-5-yl)ethanone following essentially the procedures of Example 45 (d) (165 mg, yield: 40.2%). LC-MS (020): 521.2 [M+H]⁺; Rt: 1.90 min. ¹H NMR (MeOD, 400 MHz): δ 8.50 (d, J=4.0 Hz, 2H), 7.56 (d, J=4.0 Hz, 2H), 7.43-7.34 (m, 2H), 7.19-7.15 (m, 3H), 7.01-6.92 (m, 5H), 6.80 (s, 1H), 6.72 (s, 1H), 3.95-3.94 (m, 3H), 3.33-3.32 (m, 1H), 2.65-2.64 (m, 2H), 2.31 (s, 3H), 1.94 (s, 3H).

### Example 106

### 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoic acid

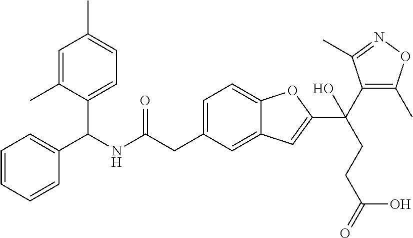 [SMILES: Cc1ccc(C(NC(=O)Cc2ccc3oc(C(O)(CCC(=O)O)c4c(C)noc4C)cc3c2)c2ccccc2)c(C)c1]

### a) ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybut-2-ynoate

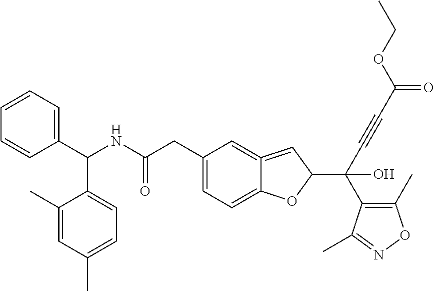

LHMDS (2.1 mL, 2.1 mmol) was added to a solution of ethyl propiolate (206 mg, 2.1 mmol) in THF (50 mL) at −78° C. under nitrogen atmosphere and the mixture was stirred at −78° C. for 1 h, then 2-(2-(3,5-dimethylisoxazole-4-carbonyl)benzofuran-5-yl)-N-((2,4-dimethylphenyl)(phenyl)methyl)acetamide (400 mg, 0.8 mmol) was added to the mixture and the temperature was allowed to warm up r.t. The reaction mixture was quenched with sat ammonia chloride, and extracted with EtOAc (50 mL×3). The combined extracts were washed with brine, dried over sodium sulfate, and concentrated under reduced pressure. The resulting residue was purified by flash column (petroleum ether/EtOAc=3/1) to afford ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybut-2-ynoate (120 mg, yield: 25%). LC-MS (P1): 591.2 [M+H]⁺; Rt: 1.55 min.

### b) ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoate

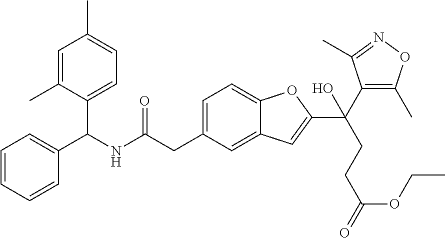 [SMILES: CCOC(=O)CCC(O)(c1cc2cc(CC(=O)NC(c3ccccc3)c3ccc(C)cc3C)ccc2o1)c1c(C)noc1C]

The mixture of ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybut-2-ynoate (30 mg) and Pd/C (5 mg) in 10 mL of ethanol was stirred under hydrogen atmosphere at rt for 3 h. The mixture was then filtered through a pad of Celite. The organic layers were concentrated to obtain ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoate (20 mg, yield: 67%). LC-MS (P1): 595.2 [M+H]⁺; Rt: 1.54 min.

### c) 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoic acid

The title compound was synthesized from ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoate following essentially the procedure of Example 31 (b) (9 mg, yield: 31%). LC-MS (038): 567.2 [M+H]⁺; Rt: 1.73 min. ¹H NMR (MeOD, 400 MHz): δ 8.70 (d, J=8.4 Hz, 1H), 7.44-7.03 (m, 9H), 6.87-6.70 (m, 4H), 6.18 (d, J=4.0 Hz, 1H), 3.56 (s, 2H), 2.68-2.42 (m, 4H), 2.24 (s, 3H), 2.16 (s, 3H), 2.05 (s, 3H), 1.93 (s, 3H).

### Example 107

### 4-(3,5-dimethylisoxazol-4-yl)-4-(5-(2-(((2,4-dimethylphenyl)(phenyl)methyl)amino)-2-oxoethyl)benzofuran-2-yl)butanoic acid

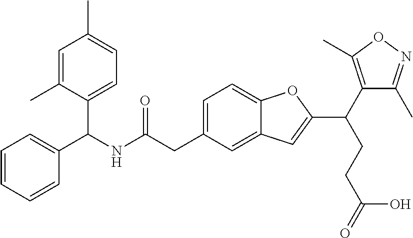 [SMILES: Cc1ccc(C(NC(=O)Cc2ccc3oc(C(CCC(=O)O)c4c(C)noc4C)cc3c2)c2ccccc2)c(C)c1]

### a) 4-(3,5-dimethylisoxazol-4-yl)-4-(5-(2-(((2,4-dimethylphenyl)(phenyl)methyl)amino)-2-oxoethyl)benzofuran-2-yl)but-3-enoic acid

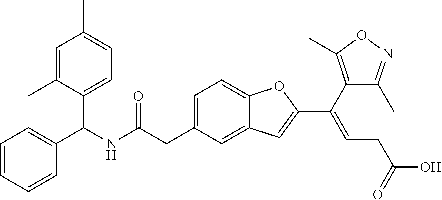 [SMILES: Cc1ccc(C(NC(=O)Cc2ccc3oc(/C(=C/CC(=O)O)c4c(C)noc4C)cc3c2)c2ccccc2)c(C)c1]

The title compound was synthesized from ethyl 4-(3,5-dimethylisoxazol-4-yl)-4-(5-(2-(((2,4-dimethylphenyl)(phenyl)methyl)amino)-2-oxoethyl)benzofuran-2-yl)-4-hydroxybutanoate following essentially the procedure of Example 58 (a) (12 mg, yield: 44%). LC-MS (027): 549.7 [M+H]⁺; Rt: 1.20 min.

### b) 4-(3,5-dimethylisoxazol-4-yl)-4-(5-(2-(((2,4-dimethylphenyl)(phenyl)methyl)amino)-2-oxoethyl)benzofuran-2-yl)butanoic acid

The title compound was synthesized from 4-(3,5-dimethylisoxazol-4-yl)-4-(5-(2-(((2,4-dimethylphenyl)(phenyl)methyl)amino)-2-oxoethyl)benzofuran-2-yObut-3-enoic acid following essentially the procedure of Example 58 (b) (3 mg, yield: 25%). LC-MS (P1): 551.7 [M+H]⁺; Rt: 1.80 min. ¹H NMR (MeOD, 400 MHz): δ 8.68 (d, J=8.0 Hz, 1H), 7.37-7.02 (m, 9H), 6.87-6.17 (m, 5H), 4.11-4.07 (m, 1H), 3.55 (s, 2H), 2.47-2.29 (m, 4H), 2.26 (s, 3H), 2.16 (s, 3H), 2.05 (s, 6H).

### Example 108

### 1-((4-chlorophenyl)(5-(2-(((S)-(2,4-dimethylphenyl)(phenyl)methyl)amino)-2-oxoethyl)benzofuran-2-yl)methoxy)cyclopropanecarboxylic acid

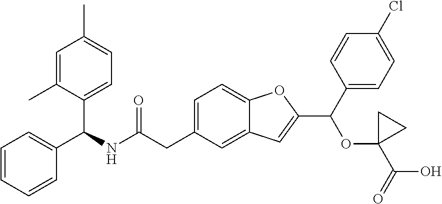 [SMILES: Cc1ccc([C@@H](NC(=O)Cc2ccc3oc(C(OC4(C(=O)O)CC4)c4ccc(Cl)cc4)cc3c2)c2ccccc2)c(C)c1]

### a) (S)-2-(2-(4-chlorobenzoyl)benzofuran-5-yl)-N-((2,4-dimethylphenyl)(phenyl)methyl)acetamide

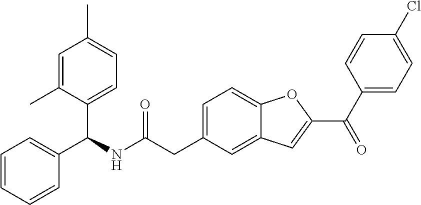 [SMILES: Cc1ccc([C@@H](NC(=O)Cc2ccc3oc(C(=O)c4ccc(Cl)cc4)cc3c2)c2ccccc2)c(C)c1]

The title compound was synthesized from (S)-(2,4-dimethylphenyl)(phenyl)methanamine, ethyl 2-(3-formyl-4-hydroxyphenyl)acetate and 2-bromo-1-(4-chlorophenyl)ethanone following essentially the procedure of Example 45 steps (a), (b) and (c). LC-MS (020): 508.1 [M+H]+; Rt: 1.60 min

### b) 2-(2-((4-chlorophenyl)(hydroxy)methyl)benzofuran-5-yl)-N—((S)-(2,4-dimethylphenyl)(phenyl)methyl)acetamide

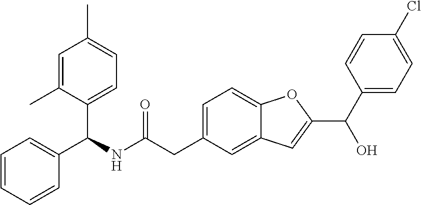 [SMILES: Cc1ccc([C@@H](NC(=O)Cc2ccc3oc(C(O)c4ccc(Cl)cc4)cc3c2)c2ccccc2)c(C)c1]

The title compound was synthesized from (S)-2-(2-(4-chlorobenzoyl)benzofuran-5-yl)-N-((2,4-dimethylphenyl)(phenyl)methyl)acetamide following essentially the procedure of Example 12 (a) (340 mg, 94.4%). LC-MS (P1): 510.1 [M+H]⁺; Rt: 1.68 min.

### c) ethyl 1-((4-chlorophenyl)(5-(2-(((S)-(2,4-dimethylphenyl)(phenyl)methyl)amino)-2-oxoethyl)benzofuran-2-yl)methoxy)cyclopropanecarboxylate

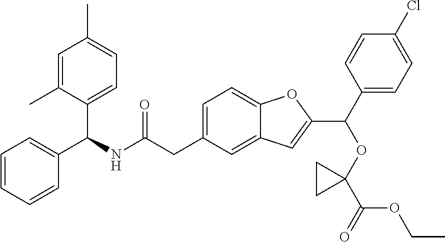 [SMILES: CCOC(=O)C1(OC(c2ccc(Cl)cc2)c2cc3cc(CC(=O)N[C@@H](c4ccccc4)c4ccc(C)cc4C)ccc3o2)CC1]

The title compound was synthesized from ethyl 1-hydroxycyclopropanecarboxylate and 2-(2-((4-chlorophenyl)(hydroxy)methyl)benzofuran-5-yl)-N—((S)-(2,4-dimethylphenyl)(phenyl)methyl)acetamide following essentially the procedure of Example 63 (b) (60 mg, 47.6%). LC-MS (P1): 622.2 [M+H]⁺; Rt: 1.70 min

### d) 1-((4-chlorophenyl)(5-(2-(((S)-(2,4-dimethylphenyl)(phenyl)methyl)amino)-2-oxoethyl)benzofuran-2-yl)methoxy)cyclopropanecarboxylic acid

The title compound was synthesized from ethyl 1-((4-chlorophenyl)(5-(2-((S)-(2,4-dimethylphenyl)(phenyl)methyl)amino)-2-oxoethyl)benzofuran-2-yl)methoxy)cyclopropanecarboxylate following essentially the procedure of Example 31 (b) (20 mg, 35.0%). LC-MS (P1): 594.2 [M+H]⁺; Rt: 1.64 min. ¹H-NMR (DMSO, 400 MHz): δ 8.87 (d, J=8.4 Hz, 1H), 7.47-7.38 (m, 6H), 7.30-7.28 (m, 3H), 7.24-7.23 (m, 3H), 6.99-6.95 (m, 3H), 6.86 (s, 1H), 6.17 (d, J=8.0 Hz, 2H), 3.57 (s, 2H), 2.22 (s, 3H), 2.14 (s, 3H), 0.98-0.92 (m, 2H), 0.65-0.60 (m, 2H)

### Example 109

### 2-(2-((4-chlorophenyl)(methoxy)methyl)benzofuran-5-yl)-N—((S)-(2,4-dimethylphenyl)(phenyl)methyl)acetamide

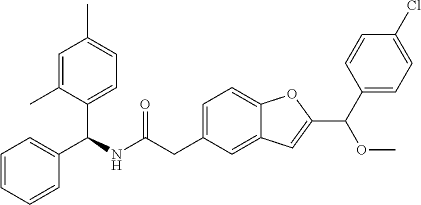 [SMILES: COC(c1ccc(Cl)cc1)c1cc2cc(CC(=O)N[C@@H](c3ccccc3)c3ccc(C)cc3C)ccc2o1]

The title compound was synthesized from 2-(2-((4-chlorophenyl)(hydroxy)methyl)benzofuran-5-yl)-N—((S)-(2,4-dimethylphenyl)(phenyl)methyl)acetamide and MeOH following essentially the procedure of Example 63 (b) (73 mg, 71.5%). LC-MS (038): 492.1 [M−OMe]⁺; Rt: 2.06 min ¹H-NMR (MeOD, 400 MHz): δ 7.49-7.46 (m, 3H), 7.41-7.35 (m, 3H), 7.28-7.22 (m, 4H), 7.14 (d, J=7.6 Hz, 2H), 6.99-6.93 (m, 3H), 6.65 (s, 1H), 6.29 (s, 1H), 5.48 (s, 1H), 3.66 (s, 2H), 3.44 (s, 3H), 2.28 (s, 3H), 2.17 (s, 3H).

### Example 110

### N-((2,4-dimethylphenyl)(phenyl)methyl)-2-(2-(1-hydroxy-1-(2-hydroxypyridin-4-yl)ethyl)furo[3,2-b]pyridin-5-yl)acetamide

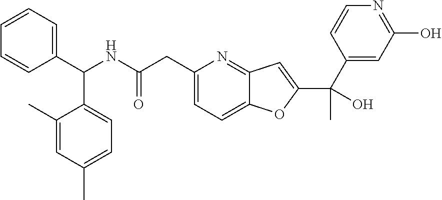 [SMILES: Cc1ccc(C(NC(=O)Cc2ccc3oc(C(C)(O)c4ccnc(O)c4)cc3n2)c2ccccc2)c(C)c1]

A mixture of ethyl 2-(2-(1-hydroxy-1-(2-hydroxypyridin-4-yOethyl)furo[3,2-b]pyridin-5-yl)acetate (80 mg, 0.24 mmol), LiOH (39 mg, 0.94 mmol) in EtOH/H₂O (10/2 mL) was stirred at rt for 1 h. The reaction mixture was then concentrated under reduced pressure. The resulting residue was dissolved in DMF (2 mL), and (2,4-dimethylphenyl)(phenyl)methanamine (53.75 mg, 0.255 mmol), EDCI (88 mg, 0.46 mmol) and HOBt (31 mg, 0.23 mmol) were added to the solution. The resulting mixture was stirred at rt overnight, and then concentrated under reduced pressure. The resulting residue was purified by Prep-HPLC to obtain N-((2,4-dimethylphenyl)(phenyl)methyl)-2-(2-(1-hydroxy-1-(2-hydroxypyridin-4-yl)ethyl)furo[3,2-b]pyridin-5-yl)acetamide (20 mg, yield: 15%). LC-MS (036): 508.1; Rₜ=1.49 min. ¹H NMR (MeOD, 400 MHz): δ 8.19 (t, J=9.2 Hz, 1H), 7.50-7.52 (m, 1H), 7.43 (d, J=6.8 Hz, 1H), 7.34-7.21 (m, 5H), 7.10-7.02 (m, 4H), 6.80 (s, 1H), 6.57 (d, J=7.2 Hz, 1H), 6.33 (s, 1H), 4.04 (s, 2H), 2.30 (s, 3H), 2.20 (s, 3H), 1.96 (s, 3H).

### Example 111

### 1-((3,5-dimethylisoxazol-4-yl)(5-(2-(((S)-(2,4-dimethylphenyl)(phenyl)methyl)amino)-2-oxoethyl)-7-fluorobenzofuran-2-yl)methoxy)cyclopropanecarboxylic acid

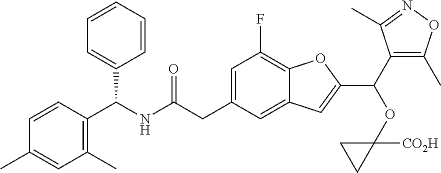

### a) ethyl 2-(3-fluoro-4-hydroxyphenyl)acetate

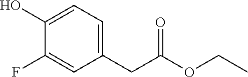

SOCl₂ (1 mL) was added drop-wise to a solution of 2-(3-fluoro-4-hydroxyphenyl)acetic acid (1 g, 5.88 mmol) in EtOH (150 mL) at 0-5° C. The reaction mixture was stirred at rt for 2 h, and concentrated udner reduced pressure to give 1.5 g of ethyl 2-(3-fluoro-4-hydroxyphenyl)acetate, which was carried through without further purification. LC-MS (020): 199.1 [M+H]⁺; Rt: 1.43 min.

### b) ethyl 2-(3-fluoro-4-hydroxy-5-iodophenyl)acetate

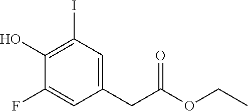

The solution of ethyl 2-(3-fluoro-4-hydroxyphenyl)acetate (1 g, 5 mmol) and NaI (752 mg, 5 mmol) in DMF (20 mL) was cooled to 10° C. An aqueous NaClO₄ solution (10%, 5.6 g) was then added drop-wise. The solution was then stirred for 2 h at 0° C. The mixture was quenched with NaHSO₃ aqueous solution while keeping the temperature below 5° C. Then the mixture stirred for 20 min at rt, and extracted with EtOAc (300 mL×3). The combined extracts were washed with brine, dried over Na₂SO₄, and concentrated under reduced pressure. The resulting residue was purified by column chromatography on silica gel (petroleum ether/EtOAc=10/1) to obtain ethyl 2-(3-fluoro-4-hydroxy-5-iodophenyl)acetate (400 mg, 24.5%). LC-MS (039): 325.0 [M+H]⁺; Rt: 1.55 min

### c) ethyl 2-(2-((3,5-dimethylisoxazol-4-yl)(hydroxy)methyl)-7-fluorobenzofuran-5-yl)acetate

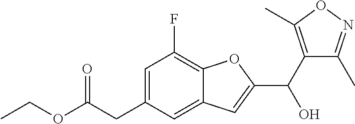 [SMILES: CCOC(=O)Cc1cc(F)c2oc(C(O)c3c(C)noc3C)cc2c1]

The solution of ethyl 2-(3-fluoro-4-hydroxy-5-iodophenyl)acetate (400 mg, 1.23 mmol), 1-(3,5-dimethylisoxazol-4-yl)prop-2-yn-1-ol (279 mg, 1.85 mmol), Pd(PPh₃)₂Cl₂ (7 mg, 0.01 mmol) and CuI (2 mg, 0.01 mmol) in Et₃N (40 mL) was stirred for 3 h at 75° C. under N₂ atmosphere. Water (100 mL) was then added to the solution, and the solution was extracted with EtOAc (50 mL×4). The combined extracts were washed with brine, dried over Na₂SO₄, and concentrated under reduced pressure. The resulting residue was purified by column chromatography on silica gel (petroleum ether/EtOAc=4/1) to obtain ethyl 2424(3,5-dimethylisoxazol-4-yl)(hydroxy)methyl)-7-fluorobenzofuran-5-yl)acetate (300 mg, 70.1%). LC-MS (044): 348.1 [M+H]⁺; Rt: 1.62 min.

### d) 2-(2-((3,5-dimethylisoxazol-4-yl)(hydroxy)methyl)-7-fluorobenzofuran-5-yl)acetic acid

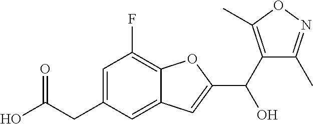 [SMILES: Cc1noc(C)c1C(O)c1cc2cc(CC(=O)O)cc(F)c2o1]

The title compound was synthesized from ethyl 2-(2-((3,5-dimethylisoxazol-4-yl)(hydroxy)methyl)-7-fluorobenzofuran-5-yl)acetate following essentially the procedure of Example 31 (b) (yield: 96.5%). LC-MS (027): 319.9 [M+H]⁺; Rt: 0.98 min.

### e) 2-(2-((3,5-dimethylisoxazol-4-yl)(hydroxy)methyl)-7-fluorobenzofuran-5-yl)-N—((S)-(2,4-dimethylphenyl)(phenyl)methyl)acetamide

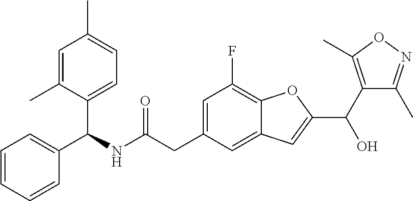 [SMILES: Cc1ccc([C@@H](NC(=O)Cc2cc(F)c3oc(C(O)c4c(C)noc4C)cc3c2)c2ccccc2)c(C)c1]

The title compound was synthesized from 2-(2-((3,5-dimethylisoxazol-4-yl)(hydroxy)methyl)-7-fluorobenzofuran-5-yl)acetic acid and (S)-(2,4-dimethylphenyl)(phenyl)methanamine following essentially the procedure of Example 2 (b) (yield: 71.6%). LC-MS (027): 512.7 [M+H]⁺; Rt: 1.20 min

### f) ethyl 1-((3,5-dimethylisoxazol-4-yl)(5-(2-(((S)-(2,4-dimethylphenyl)(phenyl)methyl)amino)-2-oxoethyl)-7-fluorobenzofuran-2-yl)methoxy)cyclopropanecarboxylate

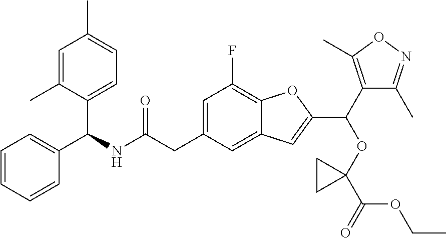 [SMILES: CCOC(=O)C1(OC(c2cc3cc(CC(=O)N[C@@H](c4ccccc4)c4ccc(C)cc4C)cc(F)c3o2)c2c(C)noc2C)CC1]

The title compound was synthesized from 2-(2-((3,5-dimethylisoxazol-4-yl)(hydroxy)methyl)-7-fluorobenzofuran-5-yl)-N—((S)-(2,4-dimethylphenyl)(phenyl)methyl)acetamide following essentially the procedure of Example 63 (b) (60 mg, yield: 33.1%). LC-MS (027): 624.7 [M+H]⁺; Rt: 1.31 min.

### g) 1-((3,5-dimethylisoxazol-4-yl)(5-(2-(((S)-(2,4-dimethylphenyl)(phenyl)methyl)amino)-2-oxoethyl)-7-fluorobenzofuran-2-yl)methoxy)cyclopropanecarboxylic acid

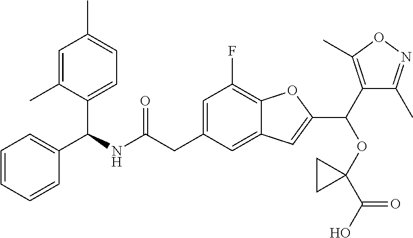 [SMILES: Cc1ccc([C@@H](NC(=O)Cc2cc(F)c3oc(C(OC4(C(=O)O)CC4)c4c(C)noc4C)cc3c2)c2ccccc2)c(C)c1]

The title compound was synthesized from ethyl 1((3,5-dimethylisoxazol-4-yl)(5-(2-((S)-(2,4-dimethylphenyl)(phenyl)methyl)amino)-2-oxoethyl)-7-fluorobenzofuran-2-yl)methoxy)cyclopropanecarboxylate following essentially the procedure of Example 31 (b) (5 mg, yield: 8.38%). LC-MS (044): 597.3 [M+H]⁺; Rt: 1.54 min. ¹H NMR (MeOD, 400 MHz): δ 7.21-7.13 (m, 4H), 7.04 (d, J=8.0 Hz, 2H), 6.94 (d, J=12.0 Hz, 1H), 6.88-6.83 (m, 3H), 6.74 (d, J=4.0 Hz, 1H), 6.19-6.17 (m, 1H), 5.92 (s, 1H), 3.54 (s, 2H), 2.34 (s, 3H), 2.16 (s, 3H), 2.06 (s, 3H), 1.93 (s, 3H), 1.2-1.15 (m, 2H), 1.09-1.07 (m, 2H).

### Example 112

### 1-((2,4-dimethyloxazol-5-yl)(5-(2-((S)-(2,4-dimethylphenyl)(phenyl)methyl)amino)-2-oxoethyl)benzofuran-2-yl)methoxy)cyclopropanecarboxylic acid

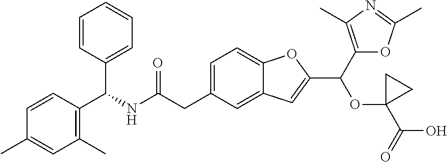 [SMILES: Cc1ccc([C@@H](NC(=O)Cc2ccc3oc(C(OC4(C(=O)O)CC4)c4oc(C)nc4C)cc3c2)c2ccccc2)c(C)c1]

### a) (2,4-dimethyloxazol-5-yl)methanol

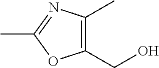

The title compound was synthesized from ethyl 2,4-dimethyloxazole-5-carboxylate following essentially the procedure of Example 9 (b) (7.05 g, yield: 79%). LCMSA038: 128.1 [M+H]⁺; Rt=1.124 min

### b) methyl 2-(2-((2,4-dimethyloxazol-5-yl)(hydroxy)methyl)benzofuran-5-yl)acetate

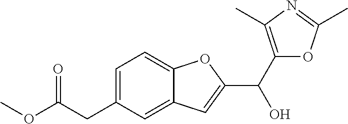

The title compound was synthesized from (2,4-dimethyloxazol-5-yl)methanol and methyl 2-(2-bromobenzofuran-5-yl)acetate following essentially the procedure of Example 15 steps (a) and (b) (898 mg, yield: 20% for two steps). LCMSA039: 316.2 [M+H]⁺; Rt=1.858 min.

### c) 2-(2-((2,4-dimethyloxazol-5-yl)(hydroxy)methyl)benzofuran-5-yl)acetic acid

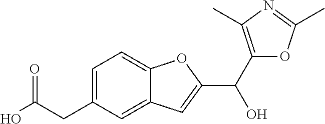 [SMILES: Cc1nc(C)c(C(O)c2cc3cc(CC(=O)O)ccc3o2)o1]

The title compound was synthesized from methyl 2-(2-(((2,4-dimethyloxazol-5-yl)(hydroxy)methyl)benzofuran-5-yl)acetate following essentially the procedure of Example 31 (b) (789 mg, crude). LCMSA036: 302.1 [M+H]⁺; Rt=1.272 min.

### d) 2-(2-((2,4-dimethyloxazol-5-yl)(hydroxy)methyl)benzofuran-5-yl)-N—((S)-(2,4-dimethylphenyl)(phenyemethypacetamide

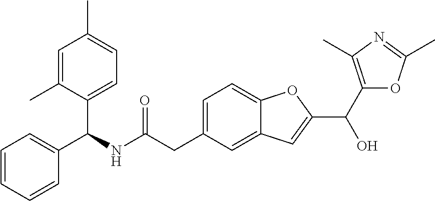 [SMILES: Cc1ccc([C@@H](NC(=O)Cc2ccc3oc(C(O)c4oc(C)nc4C)cc3c2)c2ccccc2)c(C)c1]

The title compound was synthesized from 2-(2-((2,4-dimethyloxazol-5-yl)(hydroxy)methyl)benzofuran-5-yl)acetic acid and (S)-(2,4-dimethylphenyl)(phenyl)methanamine following essentially the procedure of Example 2 (b) (530 mg, yield: 41%). LC-MS020: 495.2 [M+H]⁺; Rt=1.656 min.

### e) ethyl 1-((2,4-dimethyloxazol-5-yl)(5-(2-((S)-(2,4-dimethylphenyl)(phenyl)methyl)amino)-2-oxoethyl)benzofuran-2-yl)methoxy)cyclopropanecarboxylate

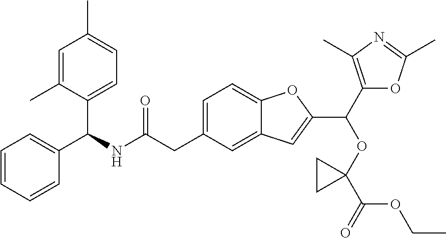 [SMILES: CCOC(=O)C1(OC(c2cc3cc(CC(=O)N[C@@H](c4ccccc4)c4ccc(C)cc4C)ccc3o2)c2oc(C)nc2C)CC1]

The title compound was synthesized from 2-(2-((2,4-dimethyloxazol-5-yl)(hydroxy)methyl)benzofuran-5-yl)-N—((S)-(2,4-dimethylphenyl)(phenyl)methyl)acetamide following essentially the procedure of Example 63 (b) (90 mg, crude). LC-MS020: 607.3 [M+H]⁺; Rt=1.820 min.

### f) 1-((2,4-dimethyloxazol-5-yl)(5-(2-((S)-(2,4-dimethylphenyl)(phenyl)methyl)amino)-2-oxoethyl)benzofuran-2-yl)methoxy)cyclopropanecarboxylic acid

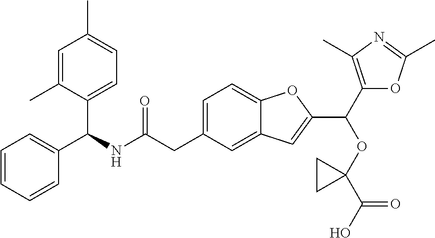 [SMILES: Cc1ccc([C@@H](NC(=O)Cc2ccc3oc(C(OC4(C(=O)O)CC4)c4oc(C)nc4C)cc3c2)c2ccccc2)c(C)c1]

The title compound was synthesized from ethyl 1((2,4-dimethyloxazol-5-yl)(5-(2-(((S)-(2,4-dimethylphenyl)(phenyl)methyl)amino)-2-oxoethyl)benzofuran-2-yl)methoxy)cyclopropanecarboxylate following essentially the procedure of Example 31 (b) (2 mg, yield: 3%). LC-MS020: 579.3 [M+H]⁺; Rt=1.466 min. ¹H NMR (CD₃Cl, 400 MHz): δ 7.47-7.45 (m, 2H), 7.22-7.19 (m, 3H), 7.04 (d, J=7.6 Hz, 2H), 6.97 (s, 1H), 6.89 (d, J=9.2 Hz, 1H), 6.74-6.73 (m, 2H), 6.36 (d, J=8.4 Hz, 1H), 6.01 (s, 1H), 5.88 (d, J=6.8 Hz, 1H), 3.71 (s, 2H), 2.46 (s, 3H), 2.27 (s, 3H), 2.20 (s, 3H), 2.16 (s, 3H), 1.26-1.15 (m, 4H).

### Example 113

### 2-(2-(3,5-dimethylisoxazol-4-yl)-1H-benzo[d]imidazol-6-yl)-N-((2,4-dimethylphenyl)(phenyl)methyl)acetamide

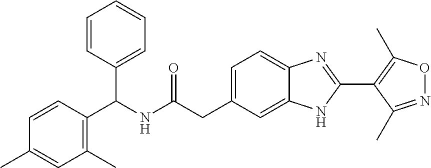 [SMILES: Cc1ccc(C(NC(=O)Cc2ccc3nc(-c4c(C)noc4C)[nH]c3c2)c2ccccc2)c(C)c1]

### a) ethyl 2-(2-(3,5-dimethylisoxazol-4-yl)-1H-benzo[d]imidazol-6-yl)acetate

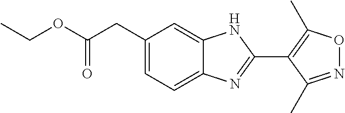 [SMILES: CCOC(=O)Cc1ccc2nc(-c3c(C)noc3C)[nH]c2c1]

The title compound was synthesized from ethyl 2-(4-aminophenyl)acetate and 3,5-dimethylisoxazole-4-carboxylic acid following essentially the procedure of Examples 70 and 71 steps (a), (b) and (c) (151 mg, yield: 49% for the 3 steps). LC-MS (010): 300.0 [M+H]⁺; Rₜ=2.00 min.

### b) 2-(2-(3,5-dimethylisoxazol-4-yl)-1H-benzo[d]imidazol-6-yl)acetic acid

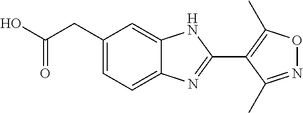 [SMILES: Cc1noc(C)c1-c1nc2ccc(CC(=O)O)cc2[nH]1]

The title compound was synthesized from ethyl 2-(2-(3,5-dimethylisoxazol-4-yl)-1H-benzo[d]imidazol-6-yl)acetate following essentially the procedure of Example 1 (f) (230 mg, crude). LC-MS (010): 272.1 [M+H]⁺; Rₜ=1.91 min.

### c) 2-(2-(3,5-dimethylisoxazol-4-yl)-1H-benzo[d]imidazol-6-yl)-N-((2,4-dimethylphenyl)(phenyl)methyl)acetamide

The title compound was synthesized from 2-(2-(3,5-dimethylisoxazol-4-yl)-1H-benzo[d]imidazol-6-yl)acetic acid and (2,4-dimethylphenyl)(phenyl)methanamine following essentially the procedure of Example 2 (b) (24 mg, yield: 17%). LC-MS022: 465.0 [M+H]⁺; Rt=1.48 min. ¹H NMR (CDCl₃, 400 MHz): δ&0.35 (d, J=8.0 Hz, 1H), 7.30 (7.24 (m, 4H), 7.13 (d, J=6.8 Hz, 2H), 7.00-6.93 (m, 4H), 6.74 (d, J=5.6 Hz, 1H), 6.31 (d, J=8.0 Hz, 1H), 3.60 (s, 2H), 2.53 (s, 3H), 2.35 (s, 3H), 2.27 (s, 3H), 2.20 (s, 3H).

### Example 114

### 2-(2-((2-amino-2-methylpropoxy)(pyridin-4-yl)methyl)benzofuran-5-yl)-N-((2,4-dimethylphenyl)(phenyl)methyl)acetamide

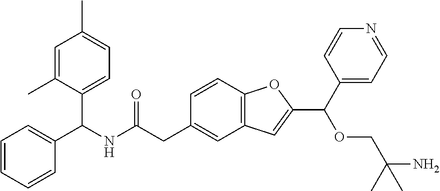 [SMILES: Cc1ccc(C(NC(=O)Cc2ccc3oc(C(OCC(C)(C)N)c4ccncc4)cc3c2)c2ccccc2)c(C)c1]

The title compound was synthesized from 2-(2-(chloro(pyridin-4-yl)methyl)benzofuran-5-yl)-N((2,4-dimethylphenyl)(phenyl)methyl)acetamide following essentially the procedure of Example 37 (a) (20 mg, yield 13%). LC-MSA026: 548.0[M+H]⁺; Rt=1.79 min. ¹H NMR (MeOD, 400 MHz): δ 8.48 (d, J=6.0 Hz, 2H), 7.63 (d, J=6.4 Hz, 2H), 7.46 (s, 1H), 7.36 (d, J=8.4 Hz, 1H), 7.29-7.19 (m, 4H), 7.13-7.12 (m, 2H), 6.98-6.92 (m, 3H), 6.63 (s, 1H), 6.27 (s, 1H), 5.32 (s, 1H), 3.65 (s, 2H), 3.39 (s, 2H), 2.27 (s, 3H), 2.16 (s, 3H), 1.07 (s, 3H), 1.03 (s, 3H).

### Example 115

### N-((4-chloro-2-methylphenyl)(pyridin-2-yl)methyl)-2-(2-(3,5-dimethylisoxazol-4-yl)benzofuran-5-yl)acetamide

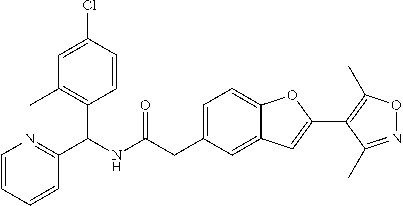 [SMILES: Cc1cc(Cl)ccc1C(NC(=O)Cc1ccc2oc(-c3c(C)noc3C)cc2c1)c1ccccn1]

### a) 2-(2-bromobenzofuran-5-yl)-N-((4-chloro-2-methylphenyl)(pyridin-2-yl)methyl)acetamide

The title compound was synthesized from (4-chloro-2-methylphenyl)(pyridin-2-yl)methanamine and 2-(2-bromobenzofuran-5-yl)acetic acid following essentially the procedure of Example 2 (b) (220 mg, yield: 51%). LC-MS (024): 469 [M+H]⁺; Rt=1.724 min.

### b) N-((4-chloro-2-methylphenyl)(pyridin-2-yl)methyl)-2-(2-(3,5-dimethylisoxazol-4-yl)benzofuran-5-yl)acetamide

The title compound was synthesized from 2-(2-bromobenzofuran-5-yl)-N-((4-chloro-2-methylphenyl)(pyridin-2-yl)methyl)acetamide following essentially the procedure of Example 11 (c) (5 mg, yield: 7%). LC-MS (012): 486 [M+H]⁺; Rt=1.831 min. ¹H NMR (DMSO-d6, 400 MHz): δ 9.02 (d, J=8.0 Hz, 1H), 8.52 (d, J=4.0 Hz, 1H), 7.79-7.75 (m, 1H), 7.53-7.51 (m, 2H), 7.35 (d, J=7.6 Hz, 1H), 7.29-7.05 (m, 6H), 6.23 (d, J=8.0 Hz, 1H), 3.64 (s, 2H), 2.66 (s, 3H), 2.48 (s, 3H), 2.20 (s, 3H).

### Example 116

### N-((4-cyano-2-methylphenyl)(phenyl)methyl)-2-(2-(1-hydroxy-1-(pyridin-4-yl)ethyl)benzofuran-5-yl)acetamide

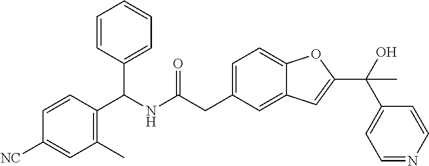 [SMILES: Cc1cc(C#N)ccc1C(NC(=O)Cc1ccc2oc(C(C)(O)c3ccncc3)cc2c1)c1ccccc1]

### a) 4-(amino(phenyl)methyl)-3-methylbenzonitrile

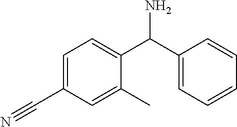 [SMILES: Cc1cc(C#N)ccc1C(N)c1ccccc1]

To a stirred solution of (4-bromo-2-methylphenyl)(phenyl)methanamine (50 mg, 0.18 mmol) in DMF (1 mL) was added cupric (I) cyanide (20 mg, 0.22 mmol) and the suspension was sealed and heated to 200° C. for 2 h. The reaction mixture was filtered through a pad of Celite and washed with CH₂Cl₂ (10 mL), EtOAc (10 mL), MeOH (10 mL) and MeCN (10 mL). The washes were combined and concentrated under reduced pressure. The concentrated residue was purified by preparative HPLC to obtain the title compound (7 mg, 18%) as a green film. LCMS-AMF: 233 [M+H]⁺; Rₜ: 4.909 min.

### b) N-((4-cyano-2-methylphenyl)(phenyl)methyl)-2-(2-(1-hydroxy-1-(pyridin-4-yl)ethyl)benzofuran-5-yl)acetamide

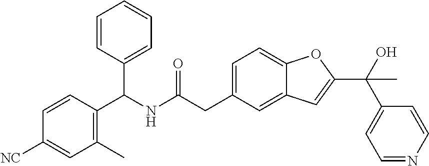 [SMILES: Cc1cc(C#N)ccc1C(NC(=O)Cc1ccc2oc(C(C)(O)c3ccncc3)cc2c1)c1ccccc1]

To a stirred suspension of 2-(2-(1-hydroxy-1-(pyrdin-4-yl)ethyl)benzofuran-5-yl)acetic acid (15 mg, 0.05 mmol) and HATU (21 mg, 0.05 mmol) in CH₂Cl₂ (0.5 mL) was added DIPEA (15 mg, 0.1 mmol). The resulting mixture was allowed to stir at rt for 15 min and then a solution of 4-(amino(phenyl)methyl)-3-methylbenzonitrile (11 mg, 0.05 mmol) in CH₂Cl₂ (0.5 mL) was added to the solution. The resulting mixture was stirred at rt for 18 h. CH₂Cl₂ (20 mL) was added to the mixture and the mixture was washed with 50% aqueous sodium bicarbonate solution (15 mL). After separating the layers, the aqueous layer was back extracted with CH₂Cl₂ (30 mL). The CH₂Cl₂ extracts were combined and concentrated under reduced pressure. The concentrated residue was purified by preparative HPLC to obtain the title compound (7 mg, 29%) as a white solid. LCMS-AMF: 502 [M+H]⁺; Rₜ: 5.326 min. ¹H NMR (400 MHz, CDCl₃) ppm 7.41 (m, 8H), 7.15 (d, J=8.0 Hz, 1H), 7.10 (q, J=4.0 Hz, 2H), 7.00 (t, J=4.0 Hz, 2H), 6.64 (s, 1H), 6.33 (d, J=8.0 Hz, 1H), 6.00 (d, J=8.0 Hz, 1H), 3.70 (s, 2H), 2.22 (s, 3H), 1.96 (s, 3H).

### Example 117

### 3-(5-(2-(((S)-(2,4-dimethylphenyl)(phenyl)methyl)amino)-2-oxoethyl)benzofuran-2-yl)-3-(pyridin-4-yl)propanoic acid

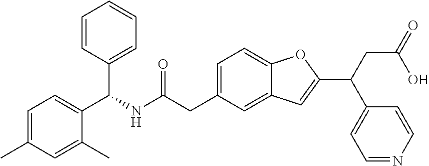 [SMILES: Cc1ccc([C@@H](NC(=O)Cc2ccc3oc(C(CC(=O)O)c4ccncc4)cc3c2)c2ccccc2)c(C)c1]

### a) (S)-ethyl 3-(5-(2-(((2,4-dimethylphenyl)(phenyl)methyl)amino)-2-oxoethyl)benzofuran-2-yl)-3-(pyridin-4-yl)acrylate

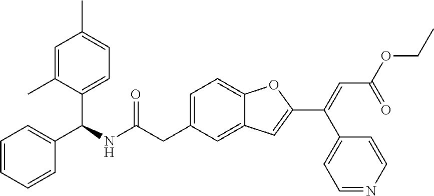 [SMILES: CCOC(=O)/C=C(\c1ccncc1)c1cc2cc(CC(=O)N[C@@H](c3ccccc3)c3ccc(C)cc3C)ccc2o1]

To a mixture of NaH (60% in mineral oil, 17 mg, 0.42 mmol) in THF (0.5 mL) at 0° C. was added triethyl phosphonoacetate (95 mg, 0.42 mmol) slowly. The reaction mixture was stirred at 0° C. for 40 min. A solution of (S)—N-((2,4-dimethylphenyl)(phenyl)methyl)-2-(2-isonicotinoylbenzofuran-5-yl)acetamide (100 mg, 0.21 mmol) in THF (0.5 mL) was added slowly to the reaction mixture at 0° C. After the addition was complete, the reaction mixture was heated to 65° C. After 2 h, the reaction was allowed to cool to rt. Once the reaction mixture at rt, the solvent was removed under reduced pressure. The resulting residue was diluted with EtOAc (35 mL) and washed with saturated aqueous NH₄Cl solution (15 mL). The aqueous layer was separated and back extracted with EtOAc (20 mL). After partitioning the layers, the EtOAc extractions were combined, washed with brine (30 mL), dried over Na₂SO₄ and concentrated under reduced pressure. The resulting oil was purified by on a silica gel column (1:1 hexanes/EtOAc) to obtain the title compound (114 mg, 99%) as a yellow oil. LCMS-TFA: 545 [M+H]⁺; Rₜ: 5.502 min.

### b) ethyl 3-(5-(2-(((S)-(2,4-dimethylphenyl)(phenyl)methyl)amino)-2-oxoethyl)benzofuran-2-yl)-3-(pyridin-4-yl)propanoate

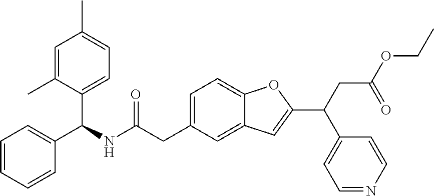 [SMILES: CCOC(=O)CC(c1ccncc1)c1cc2cc(CC(=O)N[C@@H](c3ccccc3)c3ccc(C)cc3C)ccc2o1]

To a stirring solution of (S)-ethyl 3-(5-(2-(((2,4-dimethylphenyl)(phenyl)methyl)amino)-2-oxoethyl)benzofuran-2-yl)-3-(pyridin-4-yl)acrylate (114 mg, 0.21 mmol) in EtOH (10 mL) was added Pd/C (10%, 100 mg) at rt. The reaction mixture was evacuated under reduced pressure and back flushed with H₂ and this procedure was repeated five times. After the final back flush with H₂, the reaction mixture was stirred at rt under H₂ (1 atm) for 1 h. The reaction mixture was filtered through a pad of Celite and washed with CH₂Cl₂ (10 mL), MeOH (10 mL) and MeCN (10 mL). The washes were combined and concentrated under reduced pressure. The resulting residue was used directly in the next step without further purification as a clear oil (114 mg, 99%). LCMS-AMF: 547 [M+14]⁺; Rₜ: 6.345 min.

### c) 3-(5-(2-(((S)-2,4-dimethylphenyl)(phenyl)methyl)amino)-2-oxoethyl)benzofuran-2-yl)-3-(pyridin-4-yl)propanoic acid

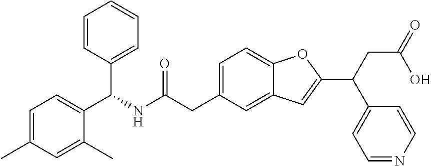 [SMILES: Cc1ccc([C@@H](NC(=O)Cc2ccc3oc(C(CC(=O)O)c4ccncc4)cc3c2)c2ccccc2)c(C)c1]

To a stirring solution of ethyl 3-(5-(2-(((S)-(2,4-dimethylphenyl)(phenyl)methyl)amino)-2-oxoethyl)benzofuran-2-yl)-3-(pyridin-4-yl)propanoate (102 mg, 0.21 mmol) in THF (1 mL) was added a solution of aqueous LiOH (1 M, 1 mL) slowly at rt. After 3 h, the reaction mixture was neutralized with HCl (4 N in 1,4-dioxane, 0.1 mL) until the pH=7. The neutralized reaction mixture was concentrated under reduced pressure. The concentrated residue was purified by preparative HPLC to obtain the title compound (10 mg, 10%) as a sticky yellow solid. LCMS-AMF: 519 [M+H]⁺; Rₜ: 4.282 min. ¹H NMR (400 MHz, CD₃OD) ppm 7.46 (s, 3H), 7.32 (d, J=8.0 Hz, 2H), 7.26 (d, J=8.0 Hz, 2H), 7.21 (d, J=8.0 Hz, 2H), 7.17 (s, 1H), 7.13 (d, J=4.0 Hz, 2H), 6.92 (m, 3H), 6.63 (s, 1H), 6.27 (s, 1H), 4.71 (t, J=4.0 Hz, 1H), 3.64 (s, 2H), 3.24 (m, 1H), 3.10 (m, 3H), 2.26 (s, 3H), 2.17 (s, 3H).

### Example 118

### N—((S)-(2,4-dimethylphenyl)(phenyl)methyl)-2-(2-(2,2,2-trifluoro-1-hydroxy-1-(pyridin-4-yl)ethyl)benzofuran-5-yl)acetamide

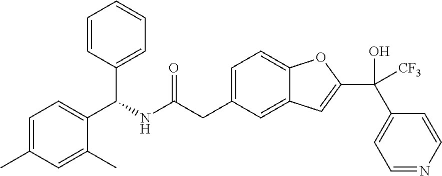 [SMILES: Cc1ccc([C@@H](NC(=O)Cc2ccc3oc(C(O)(c4ccncc4)C(F)(F)F)cc3c2)c2ccccc2)c(C)c1]

To a solution of (S)—N-((2,4-dimethylphenyl)(phenyl)methyl)-2-(2-isonicotinoylbenzofuran-5-yl)acetamide (50 mg, 0.1 mmol) in THF (2 mL) was added trifluoromethyltrimethylsilane (24 mg, 0.17 mmol) at 0° C. After 30 min at 0° C., to the stirring solution was added TBAF (1 M in THF, 0.16 mL) at 0° C. The resulting mixture was allowed to warm to rt over 14 h. EtOAc (30 mL) and water (15 mL) were added to the reaction mixture. After separating the layers, the aqueous layer was back extracted with EtOAc (30 mL). The EtOAc extracts were combined and concentrated under reduced pressure. The concentrated residue was purified by preparative HPLC to obtain the title compound (6.1 mg, 10%) as a white solid. LCMS-AMF: 545 [M+H]⁺; Rₜ: 6.098 min. ¹H NMR (400 MHz, CDCl₃) ppm 8.57 (dd, J=4.0 Hz, 2H), 7.53 (d, J=4.0 Hz, 2H), 7.36 (s, 1H), 7.27 (d, J=8.0 Hz, 1H), 7.16 (m, 4H), 7.07 (d, J=8.0 Hz, 1H), 6.91 (d, J=8.0 Hz, 1H), 6.79 (m, 2H), 6.36 (d, J=8.0 Hz, 1H), 6.04 (d, J=8.0 Hz, 1H), 5.32 (s, 1H), 3.64 (s, 2H), 2.28 (s, 3H), 2.21 (s, 3H).

### Example 119

### 2-(2-(1-hydroxy-1-(pyridin-4-yl)ethyl)benzofuran-5-yl)-N-((3-methylpyridin-4-yl)(phenyl)methyl)acetamide

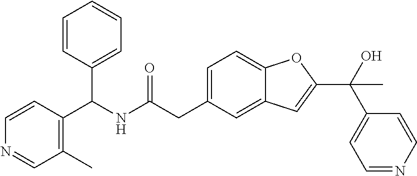 [SMILES: Cc1cnccc1C(NC(=O)Cc1ccc2oc(C(C)(O)c3ccncc3)cc2c1)c1ccccc1]

To a stirred suspension of 2-(2-(1-hydroxy-1-(pyrdin-4-yl)ethyl)benzofuran-5-yl)acetic acid (50 mg, 0.17 mmol) and HATU (77 mg, 0.2 mmol) in CH₂Cl₂ (1 mL) was added DIPEA (45 mg, 0.3 mmol). The resulting mixture was allowed to stir at rt for 20 min and then (3-methylpyridin-4-yl)(phenyl)methanamine (40 mg, 0.2 mmol) was added to the solution. The resulting mixture was stirred at rt for 13 h. CH₂Cl₂ (30 mL) was added to the mixture and the mixture was washed with 50% aqueous sodium bicarbonate solution (15 mL). After separating the layers, the aqueous layer was back extracted with CH₂Cl₂ (25 mL). The CH₂Cl₂ extracts were combined, dried over Na₂SO₄ and the solution was concentrated under reduced pressure. The concentrated residue was purified by preparative HPLC to obtain the title compound (6 mg, 8%) as a white solid. LCMS-AMF: 478 [M+H]⁺; Rₜ: 4.722 min. ¹H NMR (400 MHz, CDCl₃) ppm 8.52 (d, J=4.0 Hz, 2H), 8.23 (d, J=16.0 Hz, 2H), 7.41 (m, 3H), 7.27 (m, 3H), 7.12 (d, J=8.0 Hz, 1H), 7.01 (s, 2H), 6.88 (s, 1H), 6.61 (s, 1H), 6.36 (s, 1H), 6.26 (d, J=8.0 Hz, 1H), 3.67 (s, 2H), 2.11 (s, 3H), 1.95 (s, 3H).

### Example 120

### 2-(2-((3,5-dimethylisoxazol-4-yl)(hydroxy)methyl)benzofuran-5-yl)-1-(5-phenyl-2,3-dihydrobenzo[f][1,4]oxazepin-4(5H)-yl)ethanone

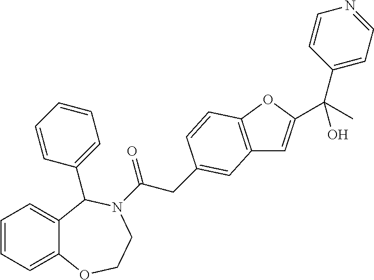 [SMILES: CC(O)(c1ccncc1)c1cc2cc(CC(=O)N3CCOc4ccccc4C3c3ccccc3)ccc2o1]

To a solution of 5-phenyl-2,3,4,5-tetrahydrobenzo[f][1,4]oxazepine (30 mg, 0.13 mmol), THF (1 mL), DMAP (32 mg, 0.27 mmol) and 2-(2-(1-hydroxy-1-(pyridin-4-yl)ethyl)benzofuran-5-yl)acetic acid (47 mg, 0.16 mmol), 127 mg T3P solution (50% w/w) was added were added dropwise and stirred at room temperature for 2 h. After the reaction was complete it was quenched with 1N sodium hydroxide. The organics were diluted with ethyl acetate and washed with water brine solution. After drying over sodium sulfate, the solvent was rotovaped off and the oil was purified by flash chromatography. (Hexanes/EtOAc gradient) and once more by reverse phase hplc to obtain the title compound (30 mg, yield 45%) as a white solid. LCMS: 505 [M+H]⁺; Rₜ=5.426 min. ¹H NMR (400 MHz, CDCl₃-d) d ppm 8.60 (br. s., 2H) 7.31-7.51 (m, 4H) 7.14-7.25 (m, 4H) 6.96-7.14 (m, 4H) 6.78-6.88 (m, 1H) 6.61-6.64 (m, 1H) 6.20 (s, 1H) 4.53-4.62 (m, 1H) 4.13-4.23 (m, 1H) 3.93-4.13 (m, 1H) 3.81-3.93 (m, 1H) 3.50-3.64 (m, 1H) 3.37-3.50 (m, 1H) 3.02-3.12 (m, 1H) 1.96 (s, 3H).

### Example 121

### 2-(2-(1-(3,5-dimethylisoxazol-4-yl)-2-hydroxyethyl)benzofuran-5-yl)-N—((S)-(2,4-dimethylphenyl)(phenyl)methyl)acetamide

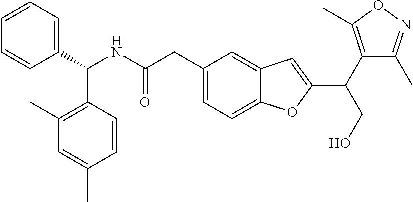 [SMILES: Cc1ccc([C@@H](NC(=O)Cc2ccc3oc(C(CO)c4c(C)noc4C)cc3c2)c2ccccc2)c(C)c1]

### a) 2-(benzyloxy)acetaldehyde

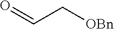 [SMILES: O=CCOCc1ccccc1]

DMSO (12.27 g, 198 mmol) was added to a solution of oxalyl dichloride (12.6 g, 99 mmol) in 120 mL of CH₂Cl₂ at −78° C. under nitrogen protection. After stirring for 15 min at the same temperature, 2-(benzyloxy)ethanol (10 g, 66 mmol) was added. The mixture was then stirred for 20 min at −78° C., then Et₃N (33.33 g, 330 mmol) was added. The resulting mixture was then allowed to warm up to rt slowly. Water (30 mL) was added to the mixture, and the mixture was extracted with CH₂Cl₂ (50 mL×3). The combined extracts were washed with brine (20 mL), dried over Na₂SO₄, and concentrated under reduced pressure. The resulting residue was purified by flash column (petroleum ether/EtOAc 3/1 to 1/1) to give 2-(benzyloxy)acetaldehyde (8.5 g, yield: 86%).

### b) 2-(b enzyloxy)-1-(3,5-dimethylisoxazol-4-yl)ethanol

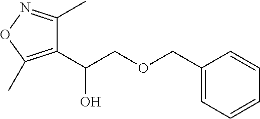 [SMILES: Cc1noc(C)c1C(O)COCc1ccccc1]

n-BuLi (58.7 mmol, 23 mL, 2.5 N in hexanes) was added drop-wise to a solution of 4-iodo-3,5-dimethylisoxazole (11.9 g, 53 mmol) in 160 mL of THF at −78° C. under. After stirring at −78° C. for 1 h, 2-(benzyloxy)acetaldehyde (8 g, 53 mmol) was added. After the addition, the mixture was allowed to warm up to rt slowly. NH₄Cl (aq.) was added to quench the mixture, and the mixture was extracted with EtOAc (70 mL×3). The combined extracts were washed with brine (20 ml), dried over Na₂SO₄, and concentrated under reduced pressure. The resulting residue was purified by flash column (petroleum ether/EtOAc=3/1 to 1/1) to give 2-(benzyloxy)-1-(3,5-dimethylisoxazol-4-yl)ethanol (3.65 g, yield: 27%). LCMSA (038): 248.2 [M+H]⁺; Rt: 1.593 min.

### c) 2-(benzyloxy)-1-(3,5-dimethylisoxazol-4-yl)ethanone

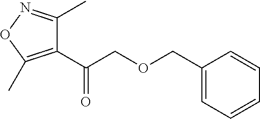 [SMILES: Cc1noc(C)c1C(=O)COCc1ccccc1]

The title compound was synthesized from 2-(benzyloxy)-1-(3,5-dimethylisoxazol-4-yl)ethanol following essentially the procedure of Example 9 (c) (2.58 g, yield: 74%). LCMSA(038): 246.1 [M+H]⁺; Rt: 1.703 min.

### d) 1-(benzyloxy)-2-(3,5-dimethylisoxazol-4-yl)-4-(triisopropylsilyl)but-3-yn-2-ol

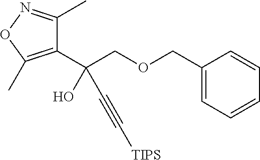

The title compound was synthesized from 2-(benzyloxy)-1-(3,5-dimethylisoxazol-4-yl)ethanone following essentially the procedure of Preparation 13 (a) (100 mg, yield: 23%). LCMSA038: 450.2[M+Na]⁺; Rt: 2.273 min.

### e) 1-(benzyloxy)-2-(3,5-dimethylisoxazol-4-yl)but-3-yn-2-ol

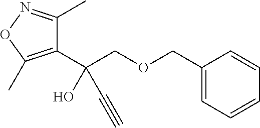 [SMILES: C#CC(O)(COCc1ccccc1)c1c(C)noc1C]

The title compound was synthesized from 1-(benzyloxy)-2-(3,5-dimethylisoxazol-4-yl)-4-(triisopropylsilyl)but-3-yn-2-ol following essentially the procedure of Preparation 13 (d) (0.935 g, yield: 89%). LCMSA038: 272.1[M+H]⁺; Rt: 1.709 min.

### f) methyl 2-(2-(2-(b enzyloxy)-1-(3,5-dimethylisoxazol-4-yl)-1-hydroxyethyl)benzofuran-5-yl)acetate

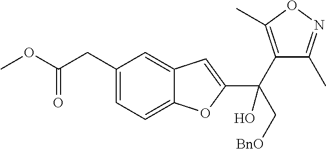

The title compound was synthesized from 1-(benzyloxy)-2-(3,5-dimethylisoxazol-4-yl)but-3-yn-2-ol and methyl 2-(4-hydroxy-3-iodophenyl)acetate following essentially the procedure of Preparation 13 (e) (150 mg, yield: 34%). LCMSA038: 436.1[M+H]⁺; Rt: 1.818 min.

### g) methyl 2-(2-(1-(3,5-dimethylisoxazol-4-yl)-2-hydroxyethyl)benzofuran-5-yl)acetate

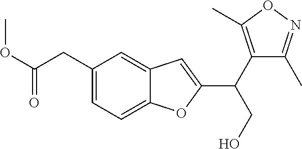 [SMILES: COC(=O)Cc1ccc2oc(C(CO)c3c(C)noc3C)cc2c1]

The title compound was synthesized from methyl 2-(2-(2-(benzyloxy)-1-(3,5-dimethylisoxazol-4-yl)-1-hydroxyethyl)benzofuran-5-yl)acetate following essentially the procedure of Examples 13 (d) and 23 (b) (15 mg, yield: 13%). LCMSA038: 330.1[M+H]⁺; Rt: 1.601 min.

### h) 2-(2-(1-(3,5-dimethylisoxazol-4-yl)-2-hydroxyethyl)benzofuran-5-yl)acetic acid

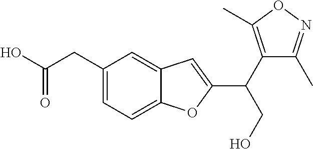 [SMILES: Cc1noc(C)c1C(CO)c1cc2cc(CC(=O)O)ccc2o1]

The title compound was synthesized from methyl 2-(2-(1-(3,5-dimethylisoxazol-4-yl)-2-hydroxyethyl)benzofuran-5-yl)acetic acid following essentially the procedure of Examples 31 (b) (15 mg, crude). LCMSA038: 316.1[M+H]⁺; Rt: 1.445 min.

### i) 2-(2-(1-(3,5-dimethylisoxazol-4-yl)-2-hydroxyethyl)benzofuran-5-yl)-N—((S)-(2,4-dimethylphenyl)(phenyl)methyl)acetamide

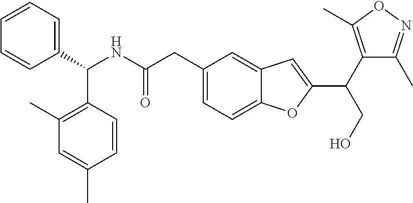 [SMILES: Cc1ccc([C@@H](NC(=O)Cc2ccc3oc(C(CO)c4c(C)noc4C)cc3c2)c2ccccc2)c(C)c1]

The title compound was synthesized from 2-(2-(1-(3,5-dimethylisoxazol-4-yl)-2-hydroxyethyl)benzofuran-5-yl)acetic acid and (S)-(2,4-dimethylphenyl)(phenyl)methanamine following essentially the procedure of Examples 2 (b) (8 mg, yield: 32% in 2 steps). LCMSA038: 509.1[M+H]⁺; Rt: 1.796 min. ¹H NMR (CDCl₃, 400 MHz): δ 7.41 (s, 1H), 7.38 (d, J=8.4 Hz, 1H), 7.25-7.18 (m, 3H), 7.16 (d, J=8.4 Hz, 1H), 7.04 (d, J=7.2 Hz, 2H), 6.96 (s, 1H), 6.89 (d, J=8.0 Hz, 1H), 6.74 (d, J=7.6 Hz, 1H), 6.47 (s, 1H), 6.36 (d, J=8.0 Hz, 1H), 5.93 (d, J=7.6 Hz, 1H), 4.30-4.20 (m, 2H), 4.07-4.03 (m, 1H), 3.69 (s, 2H), 2.34 (s, 3H), 2.27 (s, 3H), 2.19 (s, 3H), 2.18 (s, 3H).

### Examples 122, 123, 124 and 125

### 4-((R)-1-hydroxy-1-(5-(2-((R)-6-methyl-1-phenyl-3,4-dihydroisoquinolin-2(1H)-yl)-2-oxoethyl)benzofuran-2-yl)ethyl)pyridine 1-oxide, 4-((R)-1-hydroxy-1-(5-(2-((S)-6-methyl-1-phenyl-3,4-dihydroisoquinolin-2(1H)-yl)-2-oxoethyl)benzofuran-2-yOethyl)pyridine 1-oxide, 4-((S)-1-hydroxy-1-(5-(2-((R)-6-methyl-1-phenyl-3,4-dihydroisoquinolin-2(1H)-yl)-2-oxoethyl)benzofuran-2-yl)ethyl)pyridine 1-oxide, and 4-((S)-1-hydroxy-1-(5-(2-((S)-6-methyl-1-phenyl-3,4-dihydroisoquinolin-2(1H)-yl)-2-oxoethyl)benzofuran-2-yOethyl)pyridine 1-oxide

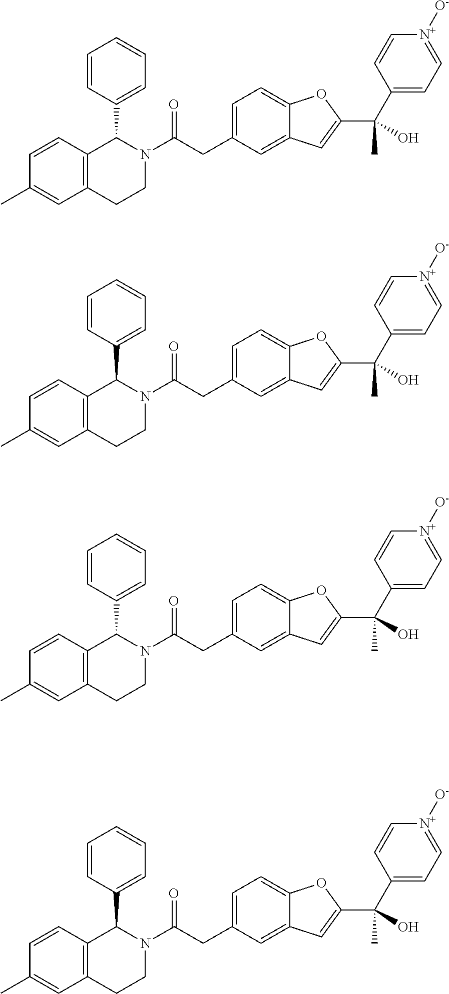 [SMILES: Cc1ccc2c(c1)CCN(C(=O)Cc1ccc3oc([C@@](C)(O)c4cc[n+]([O-])cc4)cc3c1)[C@H]2c1ccccc1 ; Cc1ccc2c(c1)CCN(C(=O)Cc1ccc3oc([C@@](C)(O)c4cc[n+]([O-])cc4)cc3c1)[C@@H]2c1ccccc1 ; Cc1ccc2c(c1)CCN(C(=O)Cc1ccc3oc([C@](C)(O)c4cc[n+]([O-])cc4)cc3c1)[C@H]2c1ccccc1 ; Cc1ccc2c(c1)CCN(C(=O)Cc1ccc3oc([C@](C)(O)c4cc[n+]([O-])cc4)cc3c1)[C@@H]2c1ccccc1]

4-(1-hydroxy-1-(5-(2-(6-methyl-1-phenyl-3,4-dihydroisoquinolin-2(1H)-yl)-2-oxoethyl)benzofuran-2-yl)ethyl)pyridine 1-oxide (450 mg) was resolved using the following method:

Instrument: Thar SFC Prep 80 (Thar Technologies, Waters); Column: ChiralCell OJ-H, 30 mm I.D.×250 mm Length, 5 μm (Daicel Chemical Industries Co., Ltd); Column Temperature: 35; Mobile Phase: CO₂/MeOH/DEA=70/30/0.1; Flow rate: 80 g/min; Back Pressure: 100 Bar; Wavelength: 214 nm; Cycle time: 22.0 min; Injection Volume: 3.0 mL; Load per injection: 75 mg; Feed solution: 450 mg dissolved in 18 mL MeOH.

Said resolution yielded 4 diastereomers:
Peak 1, 32 mg, RT: 6.2 min, 96.5% de
Peak 2, 35 mg, RT: 8.0 min, 96.5% de
Peak 3, 33 mg, RT: 9.9 min, 98.5% de
Peak 4, 44 mg, RT: 22.0 min, 100% de

### Example 126

### 1-(1,8-dimethyl-5-phenyl-2,3-dihydro-1H-benzo[e][1,4]diazepin-4(5H)-yl)-2-(2-(1-hydroxy-1-(pyridin-4-yl)ethyl)benzofuran-5-yl)ethanone

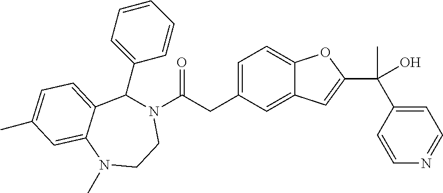 [SMILES: Cc1ccc2c(c1)N(C)CCN(C(=O)Cc1ccc3oc(C(C)(O)c4ccncc4)cc3c1)C2c1ccccc1]

### a) (2-fluoro-4-methylphenyl)(phenyl)methanol

To a stirred solution of 2-fluoro-4-methylbenzaldehyde (1.0 g, 7.24 mmol) in THF (50 mL) was added dropwise a solution of phenylmagnesium bromide (1M in THF, 7.6 mL) at 78° C. under nitrogen. The cooling bath was removed and the reaction mixture was allowed to warm to 0° C. After 2 h, the reaction mixture was diluted with water (60 mL) and diethyl ether (80 mL). The layers were separated and the aqueous layer was back extracted with diethyl ether (50 mL). After separating the layers, the diethyl ether extracts were dried over Na₂SO₄ and concentrated under reduced pressure. The resulting oil (1.56 g, 99%) was used directly in the next step without further purification. LCMS-AMF: 199 (OH frag.)[no M+H ion]⁺; Rₜ: 5.373 min.

### b) (2-fluoro-4-methylphenyl)(phenyl)methanone

To a stirred solution of (2-fluoro-4-methylphenyl)(phenyl)methanol (1.56 g, 7.24 mmol) in CH₂Cl₂ (50 mL) was added Dess-Martin periodinane (3 g, 7.24 mmol) at rt. After 90 min of stirring at rt, the reaction mixture was quenched with saturated aqueous sodium bicarbonate solution (20 mL). The layers were separated and the aqueous layer was extracted with CH₂Cl₂ (40 mL). The CH₂Cl₂ extracts were combined, washed with brine (20 mL), dried over Na₂SO₄, and concentrated under reduced pressure. The resulting oil was purified by on a silica gel column (1:1 hexanes/EtOAc) to obtain the title compound (1.13 g, 73%) as a white crystalline solid. LCMS-AMF: 215 [M+H]⁺; Rₜ: 5.919 min. ¹H NMR (400 MHz, CDCl₃) δ ppm 7.83 (d, J=8.0 Hz, 2H), 7.65 (m, 1H), 7.45 (m, 3H), 7.07 (d, J=8.0 Hz, 1H), 6.98 (d, J=12.0 Hz, 1H), 2.44 (s, 3H).

### c) 1,8-dimethyl-5-phenyl-2,3-dihydro-1H-benzo[e][1,4]diazepine

To a stirred solution of (2-fluoro-4-methylphenyl)(phenyl)methanone (250 mg, 1.17 mmol) in EtOH (2.5 mL) was added N-methylethylenediamine (340 mg, 4.67 mmol) at rt. The reaction solution was irradiated for 20 min in the microwave reactor at 180° C. The reaction mixture was concentrated under reduced pressure. The resulting oil was diluted with EtOAc (40 mL) and water (20 mL). After the layers were separated, the aqueous layer was extracted twice with EtOAc (40 mL). The EtOAc extracts were combined, washed with brine (40 mL), dried over Na₂SO₄, and concentrated under reduced pressure. The resulting oil was purified by on a silica gel column (1:1 hexanes/EtOAc) to obtain the title compound (79 mg, 27%) as a yellow solid. LCMS-AMF: 251 [M+H]⁺; Rₜ: 6.051 min. ¹H NMR (400 MHz, CDCl₃) δ ppm 7.56 (m, 2H), 7.35 (m, 3H), 6.91 (d, J=8.0 Hz, 1H), 6.76 (m, 2H), 3.74 (dd, J=8.0, 4.0 Hz, 2H), 3.61 (dd, J=8.0, 4.0 Hz, 2H), 2.79 (s, 3H), 2.39 (s, 3H).

### d) 1,8-dimethyl-5-phenyl-2,3,4,5-tetrahydro-1H-benzo[e][1,4]diazepine

To a stirred solution of 1,8-dimethyl-5-phenyl-2,3-dihydro-1H-benzo[e][1,4]diazepine (70 mg, 0.28 mmol) in MeOH (1 mL) and AcOH (17 mg, 0.28 mmol) was added sodium borohydride (11 mg, 0.28 mmol) portionwise at rt. The addition of sodium borohydride to the reaction mixture was continued until the starting material was observed to be consumed by LCMS. Once the reaction was determined to be complete by LCMS, the reaction mixture was concentrated under reduced pressure. The resulting residue was stirred in saturated aqueous sodium bicarbonate solution (10 mL) for 10 min. The mixture was diluted with EtOAc (30 mL) and water (20 mL) and the layers were later separated. The aqueous layer was extracted with EtOAc (30 mL). The EtOAc extracts were combined, dried over Na₂SO₄, and concentrated under reduced pressure. The resulting oil was purified by on a silica gel column (5% MeOH in CH₂Cl₂) to obtain the title compound (29 mg, 41%) as a yellow solid. LCMS-AMF: 253 [M+H]⁺; Rₜ: 6.202 min. ¹H NMR (400 MHz, CDCl₃) δ ppm 7.34 (m, 3H), 7.28 (m, 2H), 6.80 (s, 1H), 6.59 (d, J=8.0 Hz, 1H), 6.48 (d, J=8.0 Hz, 1H), 5.20 (s, 1H), 3.16 (m, 3H), 2.93 (s, 3H), 2.83 (m, 1H), 2.31 (s, 3H).

### e) 1-(1,8-dimethyl-5-phenyl-2,3-dihydro-1H-benzo[e][1,4]diazepin-4(5H)-yl)-2-(2-(1-hydroxy-1-(pyridine-4-yl)ethyl)benzofuran-5-yl)ethanone

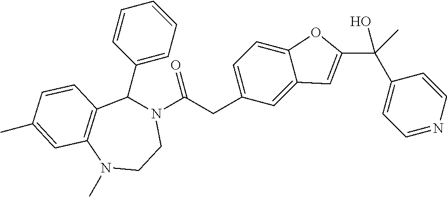 [SMILES: Cc1ccc2c(c1)N(C)CCN(C(=O)Cc1ccc3oc(C(C)(O)c4ccncc4)cc3c1)C2c1ccccc1]

To a stirred suspension of 2-(2-(1-hydroxy-1-(pyrdin-4-yl)ethyl)benzofuran-5-yl)acetic acid (34 mg, 0.11 mmol) and HATU (52 mg, 0.14 mmol) in CH₂Cl₂ (0.5 mL) was added DIPEA (30 mg, 0.23 mmol). The resulting mixture was allowed to stir at rt for 25 min and then a solution of 1,8-dimethyl-5-phenyl-2,3,4,5-tetrahydro-1H-benzo[e][1,4]diazepine (29 mg, 0.11 mmol) in CH₂Cl₂ (0.5 mL) was added to the solution. The resulting mixture was stirred at rt for 14 h. CH₂Cl₂ (20 mL) was added to the mixture and the mixture was washed with 50% aqueous sodium bicarbonate solution (10 mL). After separating the layers, the aqueous layer was back extracted with CH₂Cl₂ (20 mL). The CH₂Cl₂ extracts were combined and concentrated under reduced pressure. The concentrated residue was purified by preparative HPLC to obtain the title compound (15 mg, 25%) as a yellow solid. LCMS-AMF: 532 [M+H]⁺; Rₜ: 6.199 min. ¹H NMR (400 MHz, CDCl₃) δ ppm 7.44-6.99 (m, 14H), 6.67 (m, 2H), 6.08 (s, 1H), 4.25 (m, 1H), 3.89 (s, 2H), 3.70 (m, 1H), 3.44 (m, 1H), 3.27 (m, 1H), 2.77 (s, 3H), 2.33 (s, 3H), 1.95 (s, 3H).

Following essentially the procedure as described in Example 2, the compounds in Table 1 were prepared

TABLE 1

| Example | Structure/Name | NMR | LCMS |
|---|---|---|---|
| 127 | 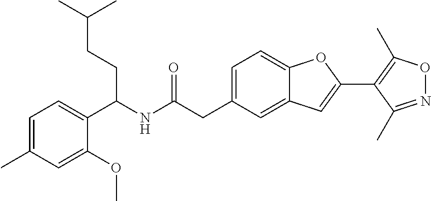 [SMILES: COc1cc(C)ccc1C(CCC(C)C)NC(=O)Cc1ccc2oc(-c3c(C)noc3C)cc2c1] 2-(2-(3,5-dimethylisoxazol-4- yl)benzofuran-5-yl)-N-(1-(2- methoxy-4-methylphenyl)-4- methylpentyl)acetamide | ¹H NMR (400 MHz, DMSO-d₆) δ ppm 8.33 (d, J = 6.8 Hz, 1H), 7.52 (t, J = 3.2 Hz, 2H), 7.23 (d, J = 7.6 Hz, 1H), 7.09 (d, J = 6.4 Hz, 1H), 7.01 (s, 1H), 6.73 (s, 1H), 6.67 (d, J = 6 Hz, 1H), 5.01 (m, 1H), 3.70 (s, 2H), 3.56 (m, 3H), 2.63 (s, 3H), 2.41 (s, 3H), 2.23 (s, 3H), 1.61 (m, 3H), 1.10 (m, 2H), 0.78 (m, 6H) | LCMS-P1: m/z 475.3 [M + H]⁺; Rₜ: 1.99 min |
| 128 | 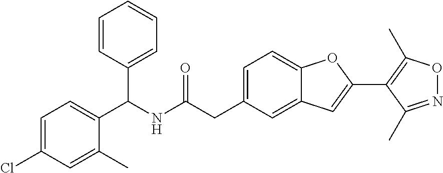 [SMILES: Cc1cc(Cl)ccc1C(NC(=O)Cc1ccc2oc(-c3c(C)noc3C)cc2c1)c1ccccc1] N-((4-chloro-2- methylphenyl)(phenyl)methyl)- 2-(2-(3,5-dimethylisoxazol-4- yl)benzofuran-5-yl)acetamide | ¹H NMR (400 MHz, DMSO-d₆) δ ppm 8.99 (d, 1H), 7.54 (d, 2H), 7.32-7.35 (m, 2H), 7.11-7.33 (m, 6H), 7.07 (s, 1H), 6.20 (d, 1H), 3.62 (s, 2H), 2.68 (s, 3H), 2.45 (s, 3H), 1.90 (s, 3H) | LCMS-X1: m/z 485.3 [M + H]⁺; Rₜ: 7.52 min |
| 129 | 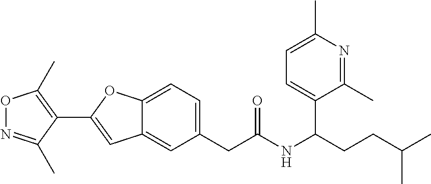 [SMILES: Cc1ccc(C(CCC(C)C)NC(=O)Cc2ccc3oc(-c4c(C)noc4C)cc3c2)c(C)n1] 2-(2-(3,5-dimethylisoxazol-4- yl)benzofuran-5-yl)-N-(1-(2,6- dimethylpyridin-3-yl)-4- methylpentyl)acetamide | ¹H NMR (DMSO, 400 MHz): δ 8.51 (d, J = 8.4 Hz, 1H), 7.48- 7.54 (m, 3H), 7.18 (dd, J = 1.6 Hz, 8 Hz, 1H), 7.03 (d, J = 7.6 Hz, 2H), 4.83 (d, J = 6 Hz, 1H), 3.50 (q, J = 7.6 Hz, 2H), 2.66 (s, 3H), 2.49 (s, 3H), 2.43 (s, 3H), 2.35 (s, 3H), 1.59-1.61 (m, 2H), 1.44-1.49 (m, 1H), 1.05-1.08 (m, 2H), 0.80-0.84 (m, 6H). | LC-MS: m/z 460.1 [M + H]⁺; Rt = 1.38 |
| 130 | 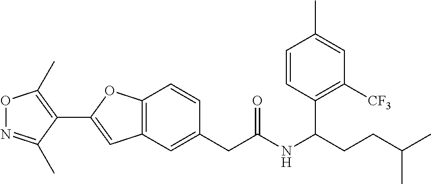 [SMILES: Cc1ccc(C(CCC(C)C)NC(=O)Cc2ccc3oc(-c4c(C)noc4C)cc3c2)c(C(F)(F)F)c1] 2-(2-(3,5-dimethylisoxazol-4- yl)benzofuran-5-yl)-N-(4- methyl-1-(4-methyl-2- (trifluoromethyl)phenyl)pentyl) acetamide | ¹H NMR (DMSO, 400 MHz): δ 8.54 (d, J = 8.4 Hz, 1H), 7.58- 7.43 (m, 5H), 7.20-7.18 (dd, J = 1.6 Hz, 8.4 Hz, 1H), 7.04 (s, 1H), 5.08-5.02 (m, 1H), 3.55 (d, J = 14 Hz, 1H), 3.49 (d, J = 14 Hz, 1H), 2.66 (s, 3H), 2.43 (s, 3H), 2.33 (s, 3H), 1.62-1.43 (m, 2H), 1.10-1.03 (m, 1H), 0.86-0.78 (m, 8H). | LC-MS: m/z 513.7 [M + H]⁺; Rt = 2. 05 min |
| 131 | 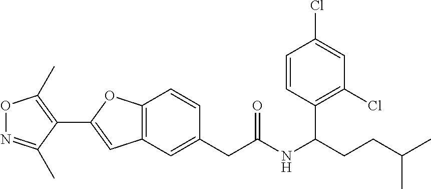 [SMILES: Cc1noc(C)c1-c1cc2cc(CC(=O)NC(CCC(C)C)c3ccc(Cl)cc3Cl)ccc2o1] N-(1-(2,4-dichlorophenyl)-4- methylpentyl)-2-(2-(3,5- dimethylisoxazol-4- yl)benzofuran-5-yl)acetamide | ¹H NMR (DMSO, 400 MHz): δ 8.67 (d, J = 5.6 Hz, 1H), 7.54 (d, J = 11.6 Hz, 3H), 7.42 (s, 2H), 7.22 (d, J = 5.6 Hz, 1H), 7.05 (s, 1H), 5.05 (s, 1H), 3.60 (m, 2H), 2.66 (s, 3H), 2.44 (s, 3H), 1.60 (s, 2H), 1.48 (s, 1H), 1.21 (s, 1H), 1.13 (s, 1H), 0.83 (m, 6H). | LC-MS: m/z 498.9 [M + H]⁺; Rt = 2.10 min |
| 132 | 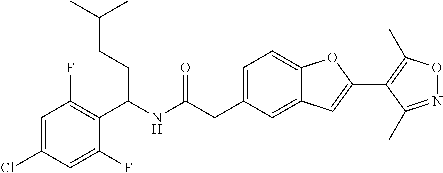 [SMILES: Cc1noc(C)c1-c1cc2cc(CC(=O)NC(CCC(C)C)c3c(F)cc(Cl)cc3F)ccc2o1] N-(1-(4-chloro-2,6- difluorophenyl)-4- methylpentyl)-2-(2-(3,5- dimethylisoxazol-4- yl)benzofuran-5-yl)acetamide | ¹H NMR (CDCl₃, 400 MHz): δ 7.48 (d, J = 8.4 Hz, 1H), 7.44 (d, J = 1.2 Hz, 1H), 7.15 (dd, J = 2.0 Hz, 8.4 Hz, 1H), 6.88 (d, J = 7.6 Hz, 1H), 6.70 (s, 1H), 5.96 (d, J = 9.2 Hz, 1H), 5.44 (q, J = 8.0 Hz, 1H), 3.69 (d, J = 16.0 Hz, 1H), 3.63 (d, J = 16.0 Hz, 1H), 2.71 (s, 3H), 2.52 (s, 3H), 1.70-1.60 (m, 1H), 1.51-1.44 (m, 1H), 1.31-1.26 (m, 1H), 1.17-1.11 (m, 1H), 1.02- 0.97 (m, 1H), 0.81 (d, J = 2.4 Hz, 3H), 0.79 (d, J = 2.4 Hz, 3H). | LCMSA024: 501.1 [M + H]⁺; Rt: 2.061 min |
| 133 | 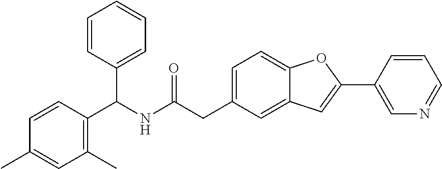 [SMILES: Cc1ccc(C(NC(=O)Cc2ccc3oc(-c4cccnc4)cc3c2)c2ccccc2)c(C)c1] N-((2,4-dimethylphenyl) (phenyl)methyl)-2-(2-(pyridin- 3-yl)benzofuran-5-yl)acetamide | ¹H NMR (400 MHz, DMSO) δ 9.144-9.148 (s, 1 H), 8.91-8.93 (d, 1 H), 8.59-8.60 (d, 1 H), 8.26- 8.28 (d, 1 H), 7.52-7.59 (m, 7 H), 7.22-7.33 (m, 4 H), 7.17-7.18 (m, 2H), 6.95-7.00 (m, 3 H), 6.18- 6.20 (d, 1 H), 3.62 (s, 2 H), 2.18 (s, 3 H), 2.15 (s, 3 H) | LCMS purity 97.90%. |
| 134 | 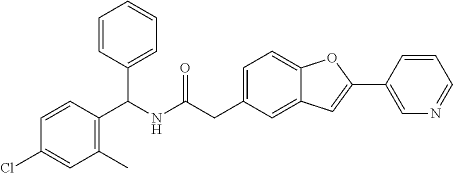 [SMILES: Cc1cc(Cl)ccc1C(NC(=O)Cc1ccc2oc(-c3cccnc3)cc2c1)c1ccccc1] N-((4-chloro-2- methylphenyl)(phenyl)methyl)- 2-(2-(pyridin-3-yl)benzofuran- 5-yl)acetamide | ¹H NMR (400 MHz, DMSO) δ 9.14-9.142 (d, 1 H), 8.99-9.02 (d, 1 H), 8.58-8.60 (dd, 1 H), 8.26- 8.29 (2, 1 H), 7.52-7.59 (m, 4 H), 7.32-7.36 (m, 2 H), 7.25-7.29 (m, 4 H), 7.18-7.23 (m, 2 H), 7.12- 7.14 (d, 1 H), 6.19-6.21 (d, 1 H), 3.60 (s, 2 H), 2.19 (s, 3 H) | LCMS purity 99.48%. |
| 135 | 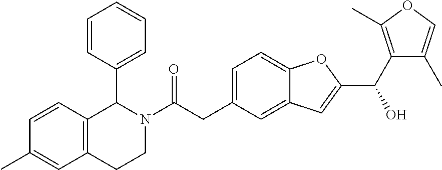 2-(2-((S)-(3,5- dimethylisoxazol-4- yl)(hydroxy)methyl)benzofuran- 5-yl)-1-(6-methyl-1-phenyl- 3,4-dihydroisoquinolin-2(1H)- yl)ethanone | ¹H NMR (MeOD, 400 MHz): δ 7.29-6.79 (m, 11H), 6.69 (s, 1H), 6.53 (s, 1H), 5.74 (s, 1H), 3.82-3.76 (m, 3H), 3.28-3.24 (m, 1H), 2.50-2.46 (m, 2H), 2.24 (s, 3H), 2.17 (s, 3H), 2.08 (s, 3H). | LC-MS (020): 507.2 [M + H]+; Rt: 1.70 min |
| 136 | 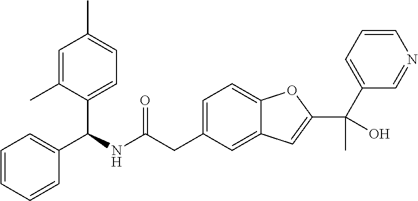 [SMILES: Cc1ccc([C@@H](NC(=O)Cc2ccc3oc(C(C)(O)c4cccnc4)cc3c2)c2ccccc2)c(C)c1] N-((S)-(2,4-dimethylphenyl) (phenyl)methyl)-2-(2-(1- hydroxy-1-(pyridin-3- yl)ethyl)benzofuran-5- yl)acetamide | ¹H NMR (DMSO, 400 MHz): δ 9.81 (s, 1H), 8.96 (s, 1H), 8.80 (d, J = 8.4 Hz, 1H), 8.63 (s, 1H), 8.28 (s, 1H), 7.62 (s, 1H), 7.32- 7.22 (m 4H), 7.15 (d, J = 7.2 Hz, 2H), 7.08 (d, J = 8.4 Hz, 1H), 6.97-6.93 (m, 3H), 6.80 (d, J = 8.4 Hz, 1H), 6.16 (d, J = 8.4 Hz, 1H), 3.38 (s, 2H), 2.22 (s, 3H), 2.14 (s, 3H), 1.77 (s, 3H). | LCMSA044: 491.70[M + H]⁺; Rt: 1.74 min |
| 137 | 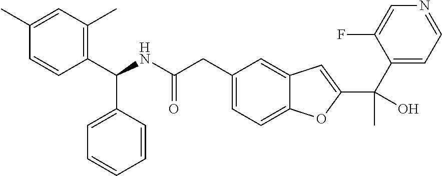 [SMILES: Cc1ccc([C@@H](NC(=O)Cc2ccc3oc(C(C)(O)c4ccncc4F)cc3c2)c2ccccc2)c(C)c1] N-((S)-(2,4- dimethylphenyl)(phenyl)methyl)- 2-(2-(1-(3-fluoropyridin-4- yl)-1-hydroxyethyl)benzofuran- 5-yl)acetamide | 1H NMR (MeOD, 400 MHz): δ 8.48 (d, J = 4.8 Hz, 1H), 8.37 (d, J = 3.2 Hz, 1H), 7.87 (dd, J₁ = 5.2 Hz, J₂ = 6.8 Hz, 1H), 7.52 (s, 1H), 7.35-7.22 (m, 5H), 7.16 (d, J = 1.2 Hz, 1H), 7.14 (s 1H), 6.99 (s, 1H), 6.94 (d, J = 2.4 Hz, 2H), 6.72 (d, J = 0.4 Hz, 1H), 6.30 (s, 1H), 3.67 (s, 2H), 2.28 (s, 3H), 2.17 (s, 3H), 2.04 (s, 3H) | LCMSA039: 509.30[M + H]⁺; Rt: 1.96 min |
| 138 | 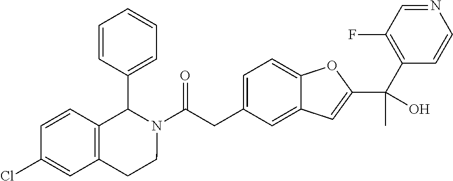 [SMILES: CC(O)(c1cc2cc(CC(=O)N3CCc4cc(Cl)ccc4C3c3ccccc3)ccc2o1)c1ccncc1F] 1-(6-chloro-1-phenyl-3,4- dihydroisoquinolin-2(1H)-yl)- 2-(2-(1-(3-fluoropyridin-4-yl)- 1-hydroxyethyl)benzofuran-5- yl)ethanone | ¹H NMR (MeOD, 400 MHz): δ 8.49 (d, J = 4.0 Hz, 1H), 8.42 (s, 1H), 7.95-7.92 (m, 1H), 7.43 (s, 1H), 7.34-7.05 (m, 10H), 6.86 (s, 1H), 6.71 (s, 1H), 4.00-3.94 (m, 3H), 3.43-3.36 (m, 1H), 2.74-2.64 (m, 2H), 2.03 (s, 3H). | LC-MS (020): 541.2 [M + H]⁺; Rt: 1.94 min |
| 139 | 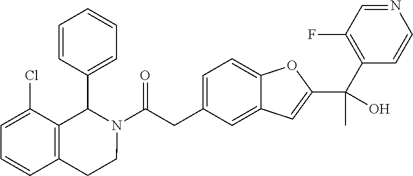 [SMILES: CC(O)(c1cc2cc(CC(=O)N3CCc4cccc(Cl)c4C3c3ccccc3)ccc2o1)c1ccncc1F] 1-(8-chloro-1-phenyl-3,4- dihydroisoquuiolin-2(1H)-yl)- 2-(2-(1-(3-fluoropyridin-4-yl)- 1-hydroxyethyl)benzofuran-5- yl)ethanone | ¹H NMR (MeOD, 400 MHz): δ 8.46 (d, J = 4.0 Hz, 1H), 8.39-8.37 (m, 1H), 7.89-7.87 (m, 1H), 7.46 (s, 1H), 7.34-6.95 (m, 11H), 6.70 (s, 1H), 4.00-3.91 (m, 3H), 3.33- 3.30 (m, 1H), 2.73-2.66 (m, 2H), 2.02 (s, 3H). | LC-MS (020): 541.2 [M + H]⁺; Rt: 1.89 min |
| 140 | 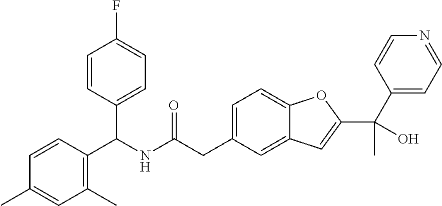 [SMILES: Cc1ccc(C(NC(=O)Cc2ccc3oc(C(C)(O)c4ccncc4)cc3c2)c2ccc(F)cc2)c(C)c1] N-((2,4-dimethylphenyl)(4- fluorophenyl)methyl)-2-(2-(1- hydroxy-1-(pyridin-4- yl)ethyl)benzofuran-5- yl)acetamide | ¹H NMR (MeOD, 400 MHz): δ 8.52 (d, J = 4.8 Hz, 2H), 7.58 (d, J = 6.0 Hz, 2H), 7.52 (s, 1H), 7.37 (d, J = 8.4 Hz, 1H), 7.24 (d, J = 7.2 Hz, 1H), 7.15 (t, J = 8.0 Hz, 2H), 7.04-6.95 (m, 5H), 6.76 (s, 1H), 6.29 (s, 1H), 3.66 (s, 2H), 2.28 (s, 3H), 2.17 (s, 3H), 1.96 (s, 3H). | LCMSA044: 509.20 [M + H]⁺; Rt = 1.85 min |
| 141 | 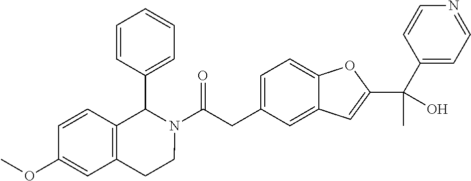 [SMILES: COc1ccc2c(c1)CCN(C(=O)Cc1ccc3oc(C(C)(O)c4ccncc4)cc3c1)C2c1ccccc1] 2-(2-(1-hydroxy-1-(pyridin-4- yl)ethyl)benzofuran-5-yl)-1-(6- methoxy-1-phenyl-3,4- dihydroisoquinolin-2(1H)- yl)ethanone | ¹H NMR (MeOD, 400 MHz): δ 8.51 (d, J = 5.6 Hz, 2H), 7.59-7.44 (m, 3H), 7.37-7.23 (m, 4H), 7.18- 7.12 (m, 3H), 6.98 (d, J = 8.4 Hz, 1H), 6.81-6.72 (m, 4H), 3.96-3.91 (m, 2H), 3.78 (s, 3H), 3.43-3.36 (m, 2H), 2.66 (s, 2H), 1.95 (s, 3H). | LCMSA(020): 518.7 [M + H]⁺; Rt: 1.81 min |
| 142 | 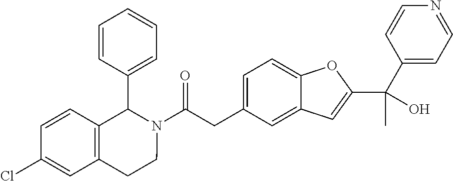 [SMILES: CC(O)(c1ccncc1)c1cc2cc(CC(=O)N3CCc4cc(Cl)ccc4C3c3ccccc3)ccc2o1] 1-(6-chloro-1-phenyl-3,4- dihydroisoquinolin-2(1H)-yl)- 2-(2-(1-hydroxy-1-(pyridin-4- yl)ethyl)benzofuran-5- yl)ethanone | ¹H NMR (MeOD, 400 MHz): δ 8.80 (s, 2H), 8.20 (s, 2H), 7.47 (s, 1H), 7.43-7.34 (m, 4H), 7.20-7.06 (m, 6H), 6.87 (d, J = 8.0 Hz, 2H), 3.96-3.92 (m, 3H), 3.45-2.38 (m, 1H), 2.75-2.66 (m, 2H), 2.03 (s, 3H). | LC-MS (020): 523.2 [M + H]⁺; Rt: 1.89 min |
| 143 | 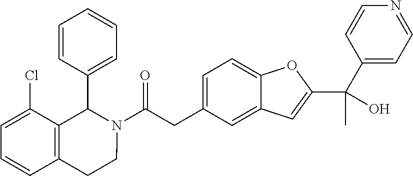 [SMILES: CC(O)(c1ccncc1)c1cc2cc(CC(=O)N3CCc4cccc(Cl)c4C3c3ccccc3)ccc2o1] 1-(8-chloro-1-phenyl-3,4- dihydroisoquinolin-2(1H)-yl)- 2-(2-(1-hydroxy-1-(pyridin-4- yl)ethyl)benzofuran-5- yl)ethanone | ¹H NMR (MeOD, 400 MHz): δ 8.79 (s, 2H), 8.18 (d, J = 4.0 Hz, 2H), 7.50 (s, 1H), 7.37-7.07 (m, 11H), 6.88 (s, 1H), 3.98-3.92 (m, 3H), 3.35-3.32 (m, 1H), 2.75-2.71 (m, 2H), 2.03 (s, 3H). | LC-MS (020): 523.2 [M + H]⁺; Rt: 1.86 min |
| 144 | 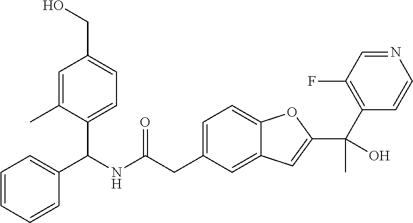 [SMILES: Cc1cc(CO)ccc1C(NC(=O)Cc1ccc2oc(C(C)(O)c3ccncc3F)cc2c1)c1ccccc1] 2-(2-(1-(3-fluoropyridin-4-yl)- 1-hydroxyethyl)benzofuran-5- yl)-N-((4-(hydroxymethyl)-2- methylphenyl)(phenyl)methyl) acetamide | ¹H NMR (DMSO, 400 MHz): δ 8.89 (d, J = 8.0 Hz, 1H), 8.50 (d, J = 4.8 Hz, 1H), 8.44 (d, J = 3.2 Hz, 1H), 7.78-7.75 (m, 1H), 7.48-7.07 (m, 11H), 6.81 (s, 1H), 6.53 (s, 1H), 6.20 (d, J = 8.0 Hz, 1H), 5.09 (t, J = 5.2 Hz, 1H), 4.42 (d, J = 5.6 Hz, 2H), 3.59 (s, 2H), 2.18 (s, 3H), 1.93 (s, 3H) | LCMSA044: 525[MH]⁺; Rt: 1.66 min |
| 145 | 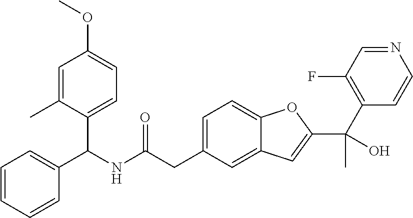 [SMILES: COc1ccc(C(NC(=O)Cc2ccc3oc(C(C)(O)c4ccncc4F)cc3c2)c2ccccc2)c(C)c1] 2-(2-(1-(3-fluoropyridin-4-yl)- 1-hydroxycthyl)benzofuran-5- yl)-N-((4-methoxy-2- methylphenyl)(phenyl)methyl) acetamide | ¹H NMR (DMSO, 400 MHz): δ 8.84 (d, J = 8.4 Hz, 1H), 8.50 (d, J = 5.2 Hz, 1H), 8.44 (d, J = 3.2Hz, 1H), 7.78-7.75 (m, 1H), 7.48 (s, 1H), 7.40-7.16 (m, 7H), 6.97 (d, J = 8.8 Hz, 1H), 6.80-6.69 (m, 3H), 6.53 (s, 1H), 6.16 (d, J = 8.4 Hz, 1H), 3.71 (s, 3H), 3.58 (s, 2H), 2.17 (s, 3H), 1.92 (s, 3H) | LCMSA044: 525[MH]⁺; Rt: 1.80 min |
| 146 | 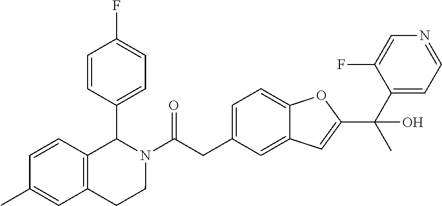 [SMILES: Cc1ccc2c(c1)CCN(C(=O)Cc1ccc3oc(C(C)(O)c4ccncc4F)cc3c1)C2c1ccc(F)cc1] 1-(1-(4-fluorophenyl)-6- methyl-3,4-dihydroisoquinolin- 2(1H)-yl)-2-(2-(1-(3- fluoropyridin-4-yl)-1- hydroxyethyl)benzofuran-5- yl)ethanone | ¹H NMR (MeOD, 400 MHz): δ 8.45 (d, J = 5.2 Hz, 1H), 8.35 (d, J = 3.2 Hz, 1H), 7.86 (s, 1H), 7.42 (s, 1H), 7.34 (d, J = 8.4 Hz, 1H), 7.20-7.15 (m, 3H), 7.03-6.92 (m, 5H), 6.80 (s, 1H), 6.68 (d, J = 4.8 Hz, 1H), 3.96-3.94 (m, 2H), 2.64 (s, 2H), 2.31 (s, 3H), 2.02 (s, 3H), 0.92-0.89 (m, 2H). | LCMS: 539.2 [M + H]⁺; Rt = 1.82 min |
| 147 | 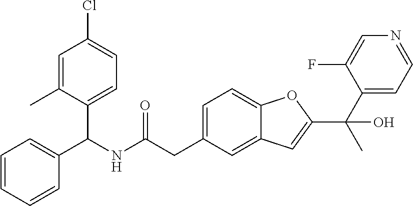 [SMILES: Cc1cc(Cl)ccc1C(NC(=O)Cc1ccc2oc(C(C)(O)c3ccncc3F)cc2c1)c1ccccc1] N-((4-chloro-2- methylphenyl)(phenyl)methyl)- 2-(2-(1-(3-fluoropyridin-4-yl)- 1-hydroxyethyl)benzofuran-5- yl)acetamide | ¹H NMR (MeOD, 400 MHz): δ 9.78 (d, J = 8.4 Hz, 1H), 9.31 (d, J = 5.2 Hz, 1H), 9.24 (s, 1H), 8.59- 8.56 (m, 1H), 8.30-7.87 (m, 9H), 7.88 (d, J = 8.8 Hz, 1H), 7.61 (s, 1H), 7.33 (s, 1H), 6.85 (d, J = 8.8 Hz, 1H), 4.41 (d, J = 5.6 Hz, 1H), 3.03 (s, 3H), 2.73 (s, 3H) | LCMS: 529.1[M + H]⁺; Rt: 1.77 min |
| 148 | 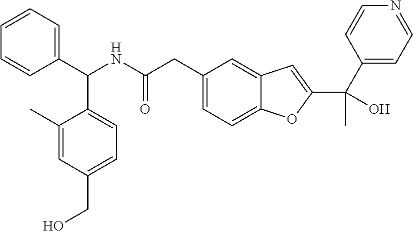 [SMILES: Cc1cc(CO)ccc1C(NC(=O)Cc1ccc2oc(C(C)(O)c3ccncc3)cc2c1)c1ccccc1] 2-(2-(1-hydroxy-1-(pyridin-4- yl)ethyl)benzofuran-5-yl)-N- ((4-(hydroxymethyl)-2- methylphenyl)(phenyl)methyl) acetamide | ¹H NMR (DMSO, 400 MHz): δ 8.75-8.65 (m, 2H), 7.84 (d, J = 6.8 Hz, 2H), 7.41-7.02 (m, 14H), 6.89 (s, 1H), 6.20 (d, J = 8.4 Hz, 1H), 4.42 (s, 2H), 3.59 (s, 2H), 2.17 (s, 3H), 1.90 (s, 3H). | LCMS (036): 507.2[M + H]⁺; Rt: 1.29 min |
| 149 | 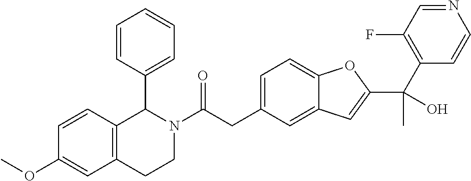 [SMILES: COc1ccc2c(c1)CCN(C(=O)Cc1ccc3oc(C(C)(O)c4ccncc4F)cc3c1)C2c1ccccc1] 2-(2-(1-(3-fluoropyridin-4-yl)- 1-hydroxyethyl)benzofuran-5- yl)-1-(6-methoxy-1-phenyl-3,4- dihydroisoquinolin-2(1H)- yl)ethanone | ¹H NMR (DMSO, 400 MHz): δ 8.50 (d, J = 4.8 Hz, 1 Hz), 8.44 (d, J = 2.8 Hz, 1H), 7.78-7.75 (m, 1H), 7.45-7.07 (m, 9H), 6.78-6.77 (m, 3H), 6.67 (s, 1H), 6.51 (s, 1H), 3.90-3.81 (m, 4H), 3.36-3.31 (m, 1H), 2.69 (s, 2H), 1.92 (s, 3H). | LCMS (038): (537.1[M + H]⁺; Rt: 1.78 min |

Following essentially the procedure as described in Example 3, the compound in Table 2 was prepared.

TABLE 2

| Example | Structure/Name | NMR | LCMS |
|---|---|---|---|
| 150 | 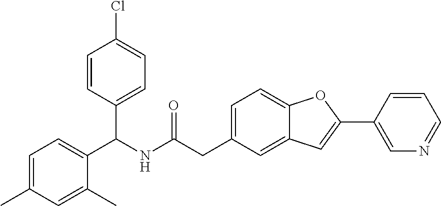 [SMILES: Cc1ccc(C(NC(=O)Cc2ccc3oc(-c4cccnc4)cc3c2)c2ccc(Cl)cc2)c(C)c1] | ¹H NMR (400 MHz, DMSO-d₆) δ ppm 9.14-9.142 (d, 1H), 8.93 (d, 1H), 8.60 (dd, 1H), 8.27 (d, 1H), 7.52-7.59 (m, 4H), 7.37- 7.39 (m, 2H), 7.26 (d, 1H), 7.20 (d, 2H), 6.96 (d, 1H), 6.19 (d, 1H), 3.62 (s, 2H), 2.15 (s, 3H) 1.99 (s, 3H). | LCMS-X1: m/z = 481.5 [M + H]⁺; Rₜ: 7.15 min |
| | N-((4-chlorophenyl)(2,4-dimethylphenyl)methyl)-2-(2- | | |
| | (pyridin-3-yl)benzofuran-5-yl)acetamide | | |

Following essentially the procedure as described in Example 10, the compounds in Table 3 were prepared.

TABLE 3

| Example | Structure/Name | NMR | LCMS |
|---|---|---|---|
| 151 | 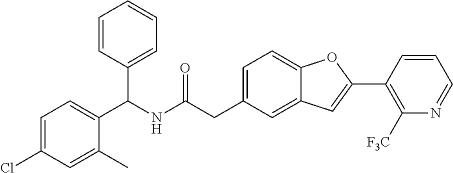 [SMILES: Cc1cc(Cl)ccc1C(NC(=O)Cc1ccc2oc(-c3cccnc3C(F)(F)F)cc2c1)c1ccccc1] | ¹H NMR (500 MHz, CDCl₃) δ ppm 8.82 (d, J = 4.8 Hz, 1H), 8.10 (s, 1H), 7.90-7.89 (m, 1H), 7.58-7.56 (m, 2H), 7.33-7.30 (m, 5H), 7.16-7.04 (m, 4H), 6.88 (d, J = 8.4 Hz, 1H), 6.35 (d, J = 8.0 Hz, 1H), 5.92 (d, J = 7.2 Hz, 1H), 3.76 (s, 2H), 3.21 (s, 3H). | LCMS-P1: m/z = 535 [M + H]⁺; Rₜ: 1.885 min |
| | N-((4-chloro-2-methylphenyl)(phenyl)methyl)- | | |
| | 2-(2-(2-(trifluoromethyl)pyridin-3- | | |
| | yl)benzofuran-5-yl)acetamide | | |
| 152 | 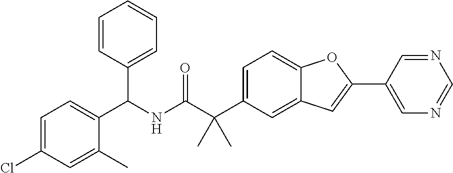 [SMILES: Cc1cc(Cl)ccc1C(NC(=O)C(C)(C)c1ccc2oc(-c3cncnc3)cc2c1)c1ccccc1] | ¹H NMR (500 MHz, DMSO-d6) δ ppm 9.34 (s, 2H), 9.21 (s, 1H), 8.14 (d, J = 8.0 Hz, 1H), 7.73 (s, 1H), 7.63 (d, J = 8.0 Hz, 2H), 7.30-7.24 (m, 5H), 7.15-7.13 (m, 1H), 7.04 (d, J = 7.0 Hz, 2H), 6.93 (d, J = 8.5 Hz, 1H), 6.29 (d, J = 7.0 Hz, 1H), 2.18 (s, 3H), 1.56 (s, 6H). | LCMS-P1: m/z = 496 [M + H]⁺; Rₜ: 1.828 min |
| | N-((4-chloro-2-methylphenyl)(phenyl)methyl)- | | |
| | 2-methyl-2-(2-(pyrimidin-5-yl)benzofuran-5- | | |
| | yl)propanamide | | |
| 153 | 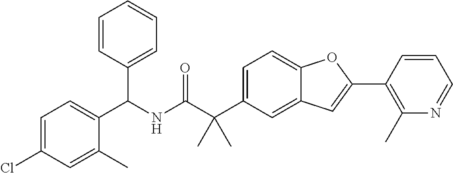 | ¹H NMR (500 MHz, DMSO-d₆) δ ppm 8.52-8.51 (m, 1H), 8.18 (d, J = 6.5 Hz, 1H), 8.12 (d, J = 8.5 Hz, 1H), 7.62-7.58 (m, 2H), 7.42 (d, J = 8.5 Hz, 1H), 7.31-7.13 (m, 6H), 7.13 (dd, J = 6.8, 1.6 Hz, 1H), 7.05 (d, J = 7.0 Hz, 2H), 6.95 (d, J = 9.0 Hz, 1H), 6.30 (d, J = 8.0 Hz, 1H), 2.76 (s, 2H), 2.50 (s, 3H), 2.19 (s, 3H), 1.56 (s, 6H). | LCMS-P1: m/z = 481 [M + H]⁺; Rₜ: 1.540 min |
| | N-((4-chloro-2-methylphenyl)(phenyl)methyl)- | | |
| | 2-methyl-2-(2-(2-methylpyridin-3-yl)benzofuran- | | |
| | 5-yl)propanamide | | |
| 154 | 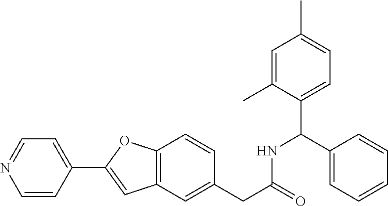 [SMILES: Cc1ccc(C(NC(=O)Cc2ccc3oc(-c4ccncc4)cc3c2)c2ccccc2)c(C)c1] | ¹H NMR (CDCl₃, 400 MHz): δ 8.71 (s, 2H), 7.74 (s, 2H), 7.54 (d, J = 8.8 Hz, 2H), 7.29 (s, 1H), 7.25-7.21 (m, 3H), 7.06 (d, J = 6.8 Hz, 2H), 6.97 (s, 1H), 6.90 (d, J = 7.2 Hz, 1H), 6.76 (d, J = 8.0 Hz, 1H), 6.38 (d, J = 8.4 Hz, 1H), 5.94 (d, J = 8.0 Hz, 1H), 3.74 (s, 2H), 2.28 (s, 3H), 2.21 (s, 3H). | LCMS-A024: 447.2 [M + H]⁺; Rt = 1.256 min. |
| | N-((2,4-dimethylphenyl)(phenyl)methyl)- | | |
| | 2-(2-(pyridin-4-yl)benzofuran-5-yl)acetamide | | |
| 155 | 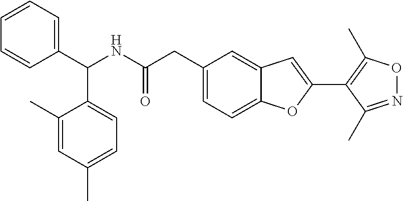 [SMILES: Cc1ccc(C(NC(=O)Cc2ccc3oc(-c4c(C)noc4C)cc3c2)c2ccccc2)c(C)c1] | ¹H NMR (400 MHz, DMSO) δ 8.90-8.92 (d, 1 H), 7.52-7.54 (d, 2 H), 7.29-7.33 (m, 2 H), 7.26 (m, 2 H), 7.22-7.24 (m, 2 H), 7.07-7.18 (m, 1 H), 6.94-6.98 (m, 3 H), 6.18-6.20 (d, 2 H), 3.61 (s, 2 H), 2.68 (s, 3 H), 2.50 (s, 2 H), 2.23 (s, 3 H), 2.15 (s, 3 H) | LCMS purity 99.57 %. |
| | 2-(2-(3,5-dimethylisoxazol-4-yl)benzofuran-5-yl)-N- | | |
| | ((2,4-dimethylphenyl)(phenyl)methyl)acetamide | | |
| 156 | 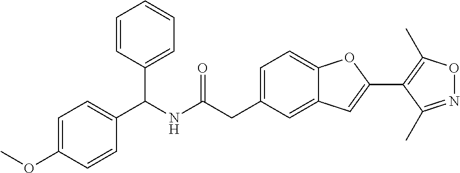 [SMILES: COc1ccc(C(NC(=O)Cc2ccc3oc(-c4c(C)noc4C)cc3c2)c2ccccc2)cc1] | ¹H NMR (400 MHz, DMSO) δ 8.97-8.99 (d, 1 H), 7.52-7.54 (d, 2 H), 7.29-7.33 (m, 2 H), 7.16-7.25 (m, 6 H), 7.07 (s, 1 H), 6.87-6.89 (d, 2 H), 6.03-6.05 (d, 1 H), 3.89 (s, 3 H), 3.72 (s, 2 H), 2.83 (s, 3 H), 2.44 (s, 3 H) | LCMS purity 99.01 %. |
| | 2-(2-(3,5-dimethylisoxazol-4-yl)benzofuran-5-yl)- | | |
| | N-((4-methoxyphenyl)(phenyl)methyl)acetamide | | |
| 157 | 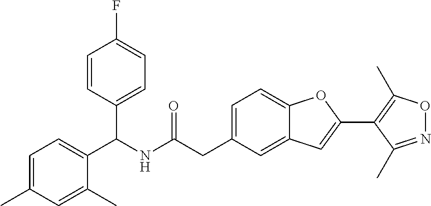 | ¹H NMR (400 MHz, DMSO) δ 8.90-8.92 (d, 1 H), 7.52-7.54 (d, H), 7.12-7.14 (m, 5 H), 7.07 (s, 1 H), 6.17-6.19 (d, 1 H), 3.61 (s, 2 H), 2.68 (s, 3 H), 2.45 (s, 3 H), 2.23 (s, 3 H), 2.14 (s, 3 H) | LCMS purity 299.79 % |
| | 2-(2-(3,5-dimethylisoxazol-4-yl)benzofuran-5-yl)-N- | | |
| | ((2,4-dimethylphenyl)(4-fluorophenyl)methyl)acetamide | | |

Following essentially the procedure as described in Example 17, the compounds in Table 4 were prepared.

TABLE 4

| Example | Structure/Name | NMR | LCMS |
|---|---|---|---|
| 158 | 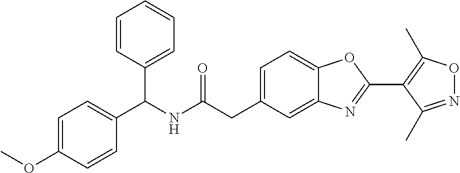 [SMILES: COc1ccc(C(NC(=O)Cc2ccc3oc(-c4c(C)noc4C)nc3c2)c2ccccc2)cc1] | ¹H NMR (400 MHz, DMSO-d₆) δ ppm 9.00 (d, 1H), 7.66-7.71 (m, 2H), 7.17-7.34 (m, 8H), 6.88 (d, 2H), 6.05 (d, 1H), 3.71 (s, 5H), 2.73 (s, 3H), 2.57 (s, 3H). | LCMS-X1: m/z = 468.6 [M + H]⁺; Rₜ: 6.65 min |
| | 2-(2-(3,5-dimethylisoxazol-4-yl)benzo[d]oxazol-5-yl)- | | |
| | N-((4-methoxyphenyl)(phenyl)methyl)acetamide | | |
| 159 | 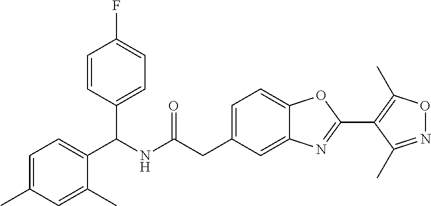 [SMILES: Cc1ccc(C(NC(=O)Cc2ccc3oc(-c4c(C)noc4C)nc3c2)c2ccc(F)cc2)c(C)c1] | ¹H NMR (400 MHz, DMSO-d₆) δ ppm 8.93 (d, 1H), 7.60-7.71 (m, 2H), 7.32 (dd, 1H), 7.12-7.22 (m, 4H), 6.97-6.98 (m, 3H), 6.18 (d, 1H), 3.66 (s, 2H), 2.82 (s, 3H), 2.67 (s, 3H), 2.23 (s, 3H), 2.14 (s, 3H). | LCMS-X1: m/z = 484.7 [M + H]⁺; Rₜ: 7.20 min |
| | 2-(2-(3,5-dimethylisoxazol-4-yl)benzo[d]oxazol-5-yl)-N- | | |
| | ((2,4-dimethylphenyl)(4-fluorophenyl)methyl)acetamide | | |
| 160 | 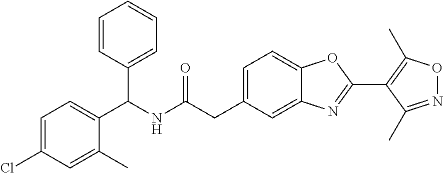 [SMILES: Cc1cc(Cl)ccc1C(NC(=O)Cc1ccc2oc(-c3c(C)noc3C)nc2c1)c1ccccc1] | ¹H NMR (400 MHz, DMSO-d₆) δ ppm 9.01 (d, 1H), 7.66-7.71 (m, 2H), 7.11-7.35 (m, 9H), 6.20 (d, 1H), 3.67 (s, 2H), 2.82 (s, 3H), 2.57 (s, 3H), 2.19 (s, 3H). | LCMS-X1: m/z = 486.3 [M + H]⁺; Rₜ: 7.33 min |
| | N-((4-chloro-2-methylphenyl)(phenyl)methyl)-2-(2-(3,5- | | |
| | dimethylisoxazol-4-yl)benzo[d]oxazol-5-yl)acetamide | | |
| 161 | 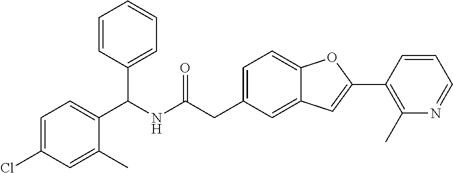 [SMILES: Cc1cc(Cl)ccc1C(NC(=O)Cc1ccc2oc(-c3cccnc3C)cc2c1)c1ccccc1] | ¹H NMR (500 MHz, DMSO-d₆) δ ppm 8.99 (d, J = 8.0 Hz, 1H), 8.57-8.56 (m, 1H), 8.32 (d, J = 8.0 Hz, 1H), 7.59-7.52 (m, 3H), 7.35-6.98 (m, 10H), 6.20 (d, J = 8.0 Hz, 1H), 3.64 (s, 2H), 2.80 (s, 3H), 2.20 (s, 3H). | LC-MS-P1: 481 [M + H]⁺; Rₜ: 1.540 min |
| | N-((4-chloro-2-methylphenyl)(phenyl)methyl)- | | |
| | 2-(2-(2-methylpyridin-3-yl)benzofuran-5-yl)acetamide | | |

Following essentially the procedure as described in Example 18, the compounds in Table 5 were prepared.

TABLE 5

| Example | Structure/Name | NMR | LCMS |
|---|---|---|---|
| 162 | 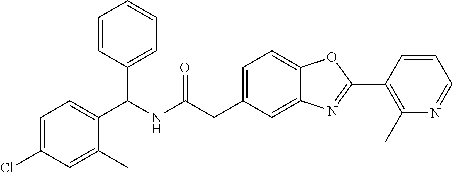 [SMILES: Cc1cc(Cl)ccc1C(NC(=O)Cc1ccc2oc(-c3cccnc3C)nc2c1)c1ccccc1] | ¹H NMR (500 MHz, DMSO-d₆) δ ppm 9.05 (d, J = 8.0 Hz, 1H), 8.69- 8.48 (m, 2H), 7.77-7.14 (m, 12H), 6.29 (d, J = 8.5 Hz, 1H), 3.71 (s, 2H), 2.97 (s, 3H), 2.22 (s, 3H). | LCMS-P1: m/z = 482 ; [M + H]⁺ Rₜ: 1.604 min |
| | N-((4-chloro-2-methylphenyl)(phenyl)methyl)- | | |
| | 2-(2-(2-methylpyridin-3-yl)benzo[d]oxazol-5- | | |
| | yl)acetamide | | |
| 163 | 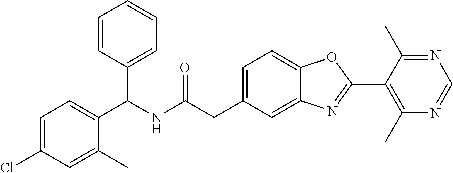 | ¹H NMR (500 MHz, DMSO-d₆) δ ppm 9.09-9.04 (m, 2H), 7.78-7.76 (m, 2H), 7.74-7.14 (m, 9H), 6.21 (d, J = 10.5, 1H), 3.71 (s, 2H), 2.54 (s, 3H), 2.50 (s, 3H), 2.21 (s, 3H). | LCMS-P1: m/z = 515 [M + H]⁺; Rₜ: 1.688 min |
| | N-((4-chloro-2-methylphenyl)(phenyl)methyl)- | | |
| | 2-(2-(4,6-dimethylpyrimidin-5-yl)benzo[d]oxazol- | | |
| | 5-yl)acetamide | | |
| 164 | 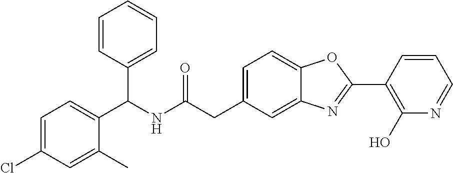 [SMILES: Cc1cc(Cl)ccc1C(NC(=O)Cc1ccc2oc(-c3cccnc3O)nc2c1)c1ccccc1] | ¹H NMR (500 MHz, DMSO-d₆) δ ppm 12.59 (d, J = 5.0 Hz, 1H), 12.11 (s, 1H), 9.87 (s, 1H), 8.94 (d, J = 8.5 Hz, 1H), 8.49-8.38 (m, 2H), 7.78-7.17 (m, 8H), 6.82-6.18 (m, 3H), 3.44 (s, 2H), 2.08 (s, 3H). | LCMS-P1: m/z = 484 [M + H]⁺; Rₜ: 1.551 min |
| | N-((4-chloro-2-methylphenyl)(phenyl)methyl)- | | |
| | 2-(2-(2-hydroxypyridin-3-yl)benzo[d]oxazol- | | |
| | 5-yl)acetamide | | |
| 165 | 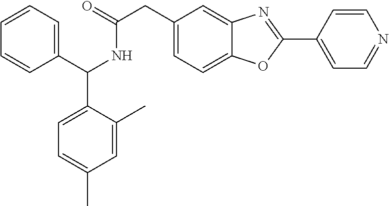 [SMILES: Cc1ccc(C(NC(=O)Cc2ccc3oc(-c4ccncc4)nc3c2)c2ccccc2)c(C)c1] | ¹H NMR (MeOH-d₄, 400 MHz): δ 8.88 (d, J = 5.6 Hz, 2H), 8.37 (d, J = 6.0 Hz, 2H), 7.81-7.49 (m, 3H), 7.33-7.17 (m, 6H), 7.00-6.97 (m, 3H), 6.31 (s, 1H), 3.77 (s, 2H), 2.28 (s, 3H), 2.19 (s, 3H). | LCMS-A012 448.2 [M + H]⁺; Rₜ: 1.48 min |
| | N-((2,4-dimethylphenyl)(phenyl)methyl)-2-(2- | | |
| | (pyridin-4-yl)benzo[d]oxazol-5-yl)acetamide | | |

Following essentially the procedure as described in Example 24, the compound in Table 6 was prepared.

TABLE 6

| Example | Structure/Name | NMR |
|---|---|---|
| 166 | 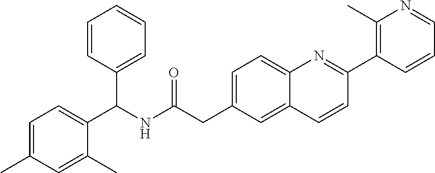 [SMILES: Cc1ccc(C(NC(=O)Cc2ccc3nc(-c4cccnc4C)ccc3c2)c2ccccc2)c(C)c1] | ¹H NMR (400 MHz, CDCl₃) δ ppm 8.61 (dd, J = 4.8, 1.5 Hz, 1H), 8.20 (d, J = 8.3 Hz, 1H), 8.14 (d, J = 8.8 Hz, 1H), 7.85 (dd, J = 7.7, 1.4 Hz, 1H), 7.79 (s, 1H), 7.69 (dd, J = 8.7, 1.9 Hz, 1H), 7.57 (d, J = 8.5 Hz, 1H), 7.31-7.23 (m, 4H), 7.11 (d, J = 6.5 Hz, 2H), 6.99 (s, 1H), 6.92 (d, J = 7.8 Hz, 1H), 6.82 (d, J = 7.8 Hz, 1H), 6.42 (d, J = 8.0 Hz, 1H), 6.04 (d, J = 8.0 Hz, 1H), 3.85 (s, 2H), 2.67 (s, 3H), 2.29 (s, 3H), 2.24 (s, 3H) |
| | N-((2,4-dimethylphenyl)(phenyl)methyl)- | |
| | 2-(2-(2-methylpyridin-3-yl)quinolin-6-yl)acetamide | |

Following essentially the procedure as described in Example 28, the compounds in Table 7 were prepared.

TABLE 7

| Example | Structure/Name | NMR | LCMS |
|---|---|---|---|
| 167 | 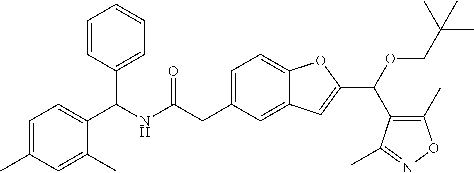 [SMILES: Cc1ccc(C(NC(=O)Cc2ccc3oc(C(OCC(C)(C)C)c4c(C)noc4C)cc3c2)c2ccccc2)c(C)c1] | ¹H NMR (400 MHz, MeOD-d₄) δ ppm 7.52 (s, 1H), 7.38 (d, J = 8.8 Hz, 1H), 7.24-7.28 (m, 6H), 6.93- 7.15 (m, 3H), 6.74 (s, 1H), 6.29 (s, 1H), 5.53 (s, 1H), 3.67 (s, 2H), 3.23 (d, J = 8.0 Hz, 2H), 2.44 (s, 3H), 2.27 (s, 3H), 2.24 (s, 3H), 2.17 (s, 3H), 0.97 (s, 9H). | LCMS-P1: m/z = 565.3 [M + H]⁺; Rₜ: 2.25 min |
| | 2-(2-((3,5-dimethylisoxazol-4- | | |
| | yl)(neopentyloxy)methyl)benzofuran-5-yl)-N- | | |
| | ((2,4-dimethylphenyl)(phenyl)methyl)acetamide | | |
| 168 | 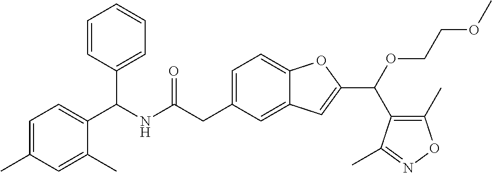 | ¹H NMR (400 MHz, DMSO-d₆) δ ppm 8.87 (d, J = 8.0 Hz, 1H), 7.47-7.44 (m, 2H), 7.29-7.14 (m, 6H), 6.96-6.93 (m, 3H), 6.79 (s, 1H), 6.17 (d, J = 8.4 Hz, 1H), 5.70 (s, 1H), 3.61-3.48 (m, 6H), 3.24 (s, 3H), 2.37 (s, 3H), 2.21 (s, 3H), 2.15 (s, 3H), 2.13 (s, 3H). | LCMS-P1: m/z = 553.2 [M + H]⁺; Rₜ: 1.788 min |
| | 2-(2-((3,5-dimethylisoxazol-4-yl)(2- | | |
| | methoxyethoxy)methyl)benzofuran-5-yl)-N- | | |
| | ((2,4-dimethylphenyl)(phenyl)methyl)acetamide | | |
| 169 | 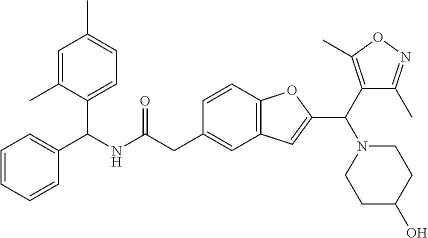 | ¹H NMR (DMSO-d₆, 400 MHz): δ 8.88 (d, J = 8.0 Hz, 2H), 7.43- 7.41 (m, 2H), 7.30-7.22 (m, 3H), 7.15-7.14 (m, 3H), 6.98-6.91 (m, 3H), 6.84 (s, 1H), 6.16 (d, J = 8.4 Hz, 1H) 4.64 (s, 1H), 4.58 (s, 1H), 3.56 (s, 1H), 3.33 (s, 1H), 2.69-2.66 (m, 2H), 2.39 (s, 3H), 2.21 (s, 3H), 2.20 (s, 3H), 2.13 (s, 3H), 2.11-2.00 (m, 2H), 1.71-1.70 (m, 2H), 1.43-1.39 (m, 2H). | LCMS-A012: No mass, [M + H]⁺; Rt = 1.82 min. |
| | 2-(2-((3,5-dimethylisoxazol-4-yl)(4- | | |
| | hydroxypiperidin-1-yl)methyl)benzofuran-5-yl)- | | |
| | N-((2,4-dimethylphenyl)(phenyl)methyl)acetamide | | |
| 170 | 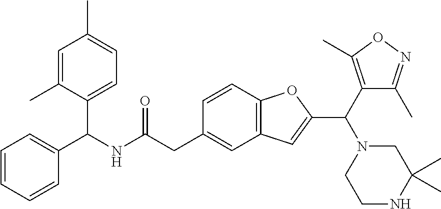 | ¹H NMR (MeOH-d₄, 400 MHz): δ 7.49 (s, 1H), 7.38 (d, J = 8.4 Hz, 1H), 7.29-7.21 (m, 4H), 7.15-7.13 (m, 2H), 6.98-6.90 (m, 3H), 6.78 (s, 1H), 6.28 (s, 1H), 4.51 (s, 1H), 3.66 (s, 2H), 3.30 (br, 4H), 2.95 (s, 2H), 2.48 (s, 3H), 2.36 (s, 3H), 2.27 (s, 3H), 2.16 (s, 3H), 1.19 (s, 6H). | LCMS-A012: No mass [M + H]⁺; Rt = 2.03 min, |
| | 2-(2-((3,5-dimethylisoxazol-4-yl)(3,3- | | |
| | dimethylpiperazin-1-yl)methyl)benzofuran-5-yl)- | | |
| | N-((2,4-dimethylphenyl)(phenyl)methyl)acetamide | | |
| 171 | 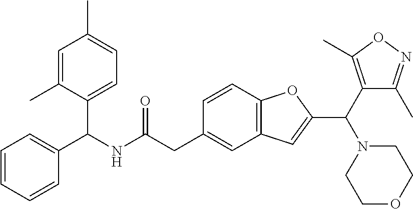 | ¹H NMR (MeOH-d₄, 400 MHz): δ 7.45 (s, 1H), 7.31-7.25 (m, 4H), 7.18-7.14 (m, 2H), 7.00-6.91 (m, 4H), 6.30 (s, 1H), 5.60 (s, 1H), 5.07 (s, 1H), 3.68 (s, 4H), 3.59 (s, 2H), 2.71-2.70 (m, 2H), 2.55-2.53 (m, 2H), 2.44 (s, 3H), 2.28 (s, 3H), 2.27 (s, 3H), 2.19-2.17 (m, 3H). | LCMS-A012: No mass, [M + H]⁺; Rt = 1.90 min. |
| | 2-(2-((3,5-dimethylisoxazol-4- | | |
| | yl)(morpholino)methyl)benzofuran-5-yl)-N- | | |
| | ((2,4-dimethylphenyl)(phenyl)methyl)acetamide | | |
| 172 | 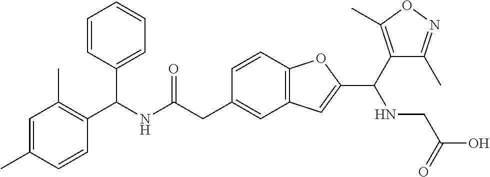 | ¹H NMR (MeOD, 400 MHz): δ 7.49 (s, 1H), 7.35 (d, J = 8.4 Hz, 1H), 7.29-7.19 (m, 4H), 7.13 (d, J = 6.8 Hz, 2H), 6.98 (s, 1H), 6.93 (m, 2H), 6.73 (s, 1H), 6.28 (s, 1H), 5.14 (s, 1H), 3.66 (s, 2H), 3.23 (s, 2H), 2.43 (s, 3H), 2.27 (s, 3H), 2.23 (m, 3H), 2.16 (s, 3H). | LC-MS: no mass [M + H]⁺; fragment at m/z 477. Rt = 1.72 min |
| | 2-(((3,5-dimethylisoxazol-4-yl)(5-(2-(((2,4- | | |
| | dimethylphenyl)(phenyl)methyl)amino)-2- | | |
| | oxoethyl)benzofuran-2-yl)methyl)amino)acetic acid | | |
| 173 | 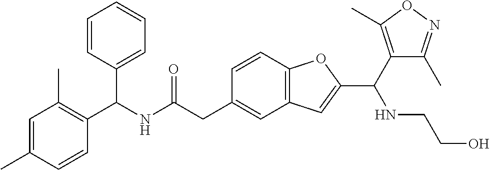 | ¹H NMR (DMSO, 400 MHz): δ 8.86 (d, J = 8.0 Hz, 1H), 7.44 (s, 1H), 7.40 (d, J = 8.8 Hz, 1H), 7.27-7.31 (m, 3H), 7.14-7.21 (m, 3H), 6.93-6.98 (m, 3H), 6.75 (s, 1H), 6.15 (d, J = 8.4 Hz, 1H), 5.01 (s, 1H), 4.58 (t, J = 5.2 Hz, 1H), 3.56 (s, 2H), 3.52-3.48 (m, 2H), 2.55-2.50 (m, 2H), 2.36 (s, 3H), 2.21 (s, 3H), 2.14 (s, 6H). | LC-MSA012: no mass [M + H]⁺; fragment at m/z 477. Rt = 1.65 min |
| | 2-(2-((3,5-dimethylisoxazol-4-yl)((2- | | |
| | hydroxyethyl)amino)methyl)benzofuran-5-yl)-N- | | |
| | ((2,4-dimethylphenyl)(phenyl)methyl)acetamide | | |
| 174 | 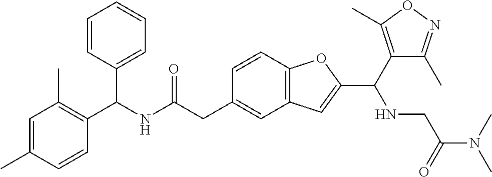 | ¹H NMR (MEOD, 400 MHz): δ 7.50 (s, 1H), 7.36 (d, J = 8.4 Hz, 1H), 7.20-7.36 (m, 4H), 7.13-7.15 (m, 2H), 6.93-6.98 (m, 3H), 6.73 (s, 1H), 6.29 (s, 1H), 5.10 (s, 1H), 3.66 (s, 2H), 3.52 (s, 2H), 2.95 (s, 6H), 2.43 (s, 3H), 2.27 (s, 3H), 2.24 (s, 3H), 2.16 (s, 3H). | LC-MS: no mass [M + H]⁺; fragment at m/z 477; Rt = 1.99 min |
| | 2-(((3,5-dimethylisoxazol-4-yl)(5-(2-(((2,4- | | |
| | dimethylphenyl)(phenyl)methyl)amino)-2- | | |
| | oxoethyl)benzofuran-2-yl)methyl)amino)-N,N- | | |
| | dimethylacetamide | | |
| 175 | 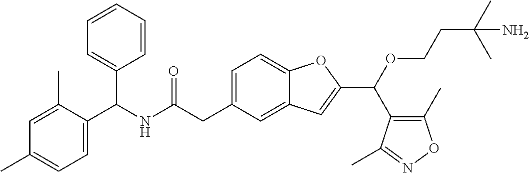 | ¹H NMR (MeOD, 400 MHz): δ 7.46 (s, 1H), 7.36 (d, J = 8.4 Hz, 1H), 7.30-7.19 (m, 4H), 7.14-7.13 (m, 2H), 6.98-6.90 (m, 3H), 6.64 (s, 1H), 6.28 (s, 1H), 5.17 (s, 1H), 3.74 (t, J = 6.8 Hz, 2H), 3.65 (s, 2H), 2.46 (s, 3H), 2.28 (s, 3H), 2.27 (s, 3H), 2.16 (s, 3H), 1.75- 1.71 (m, 2H), 1.15 (s, 6H). | LC-MSA011: no mass [M + H]⁺; fragment at m/z 477; Rₜ = 1.54 min |
| | 2-(2-((3-amino-3-methylbutoxy)(3,5- | | |
| | dimethylisoxazol-4-yl)methyl)benzofuran-5-yl)-N- | | |
| | ((2,4-dimethylphenyl)(phenyl)methyl)acetamide | | |

Following essentially the procedure as described in Example 31, the compounds in Table 8 were prepared.

TABLE 8

| Example | Structure/Name | NMR | LCMS |
|---|---|---|---|
| 176 | 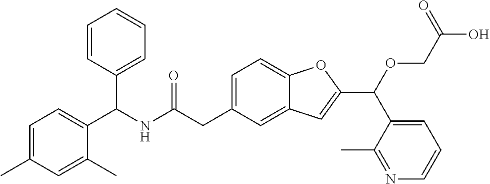 [SMILES: Cc1ccc(C(NC(=O)Cc2ccc3oc(C(OCC(=O)O)c4cccnc4C)cc3c2)c2ccccc2)c(C)c1] | ¹H NMR (400 MHz, DMSO-d₆) ppm 8.91 (d, J = 8.0 Hz, 1H), 8.41 (dd, J = 4.8, 1.6 Hz, 1H), 7.92 (dd, J = 8.0, 1.6 Hz, 1H), 7.48 (s, 1H), 7.42 (d, J = 8.4 Hz, 1H), 7.48 (s, 1H), 7.42 (d, J = 8.4 Hz, 1H), 7.33-7.15 (m, 7H), 7.00-6.92 (m, 3H), 6.82 (s, 1H), 6.17 (d, J = 8.8 Hz, 1H), 6.09 (s, 1H), 3.92 (s, 2H), 3.58 (s, 2H), 2.46 (s, 3H), 2.22 (s, 3H), 2.14 (s, 3H). | LCMS-P1: m/z = 549.0 [M + H]⁺; Rₜ: 1.507 min |
| | 2-((5-(2-(((2,4-dimethylphenyl)(phenyl)methyl) | | |
| | amino)-2-oxoethyl)benzofuran-2-yl)(2-methylpyridin- | | |
| | 3-yl)methoxy)acetic acid | | |
| 177 | 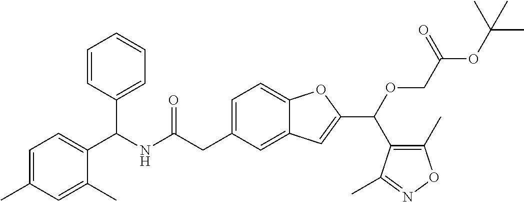 [SMILES: Cc1ccc(C(NC(=O)Cc2ccc3oc(C(OCC(=O)OC(C)(C)C)c4c(C)noc4C)cc3c2)c2ccccc2)c(C)c1] | ¹H NMR (400 MHz, CDCl₃) δ ppm 8.89 (d, J = 8.4 Hz, 1H), 7.50- 7.46 (m, 2H), 7.32-7.15 (m, 6H), 7.00-6.87 (m, 4H), 6.18 (d, J = 8.0 Hz, 1H), 5.80 (s, 1H), 4.09 (s, 2H), 3.59 (s, 2H), 2.41 (s, 3H), 2.22 (s, 3H), 2.16 (s, 3H), 2.14 (s, 3H). | LCMS-P1: m/z = 609 [M + H]⁺; Rₜ: 1.985 min |
| | tert-butyl 2-((3,5-dimethylisoxazol-4-yl)(5-(2- | | |
| | (((2,4-dimethylphenyl)(phenyl)methyl)amino)-2- | | |
| | oxoethyl)benzofuran-2-yl)methoxy)acetate | | |
| 178 | 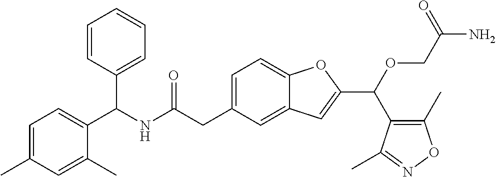 [SMILES: Cc1ccc(C(NC(=O)Cc2ccc3oc(C(OCC(N)=O)c4c(C)noc4C)cc3c2)c2ccccc2)c(C)c1] | ¹H NMR (400 MHz, DMSO-d₆) ppm 8.88 (d, J = 8.4 Hz, 1H), 7.49- 7.44 (m, 2H), 7.31-7.14 (m, 8H), 6.99-6.94 (m, 4H), 6.17 (d, J = 8.4 Hz, 1H), 5.79 (s, 1H), 3.91- 3.88 (m, 2H), 3.58 (s, 2H), 2.40 (s, 3H), 2.21 (s, 3H), 2.15 (s, 3H), 2.13 (s, 3H). | LCMS-P1: m/z = 574.2 [M + Na]⁺; Rₜ: 1.734 min |
| | 2-(2-((2-amino-2-oxoethoxy)(3,5-dimethylisoxazol-4- | | |
| | yl)methyl)benzofuran-5-yl)-N-((2,4- | | |
| | dimethylphenyl)(phenyl)methyl)acetamide | | |
| 179 | 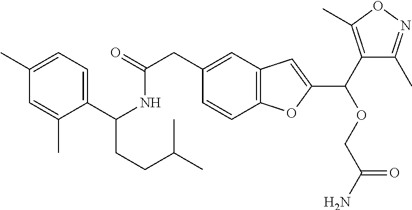 | ¹H NMR (MeOH-d₄, 400 MHz): δ 7.39 (s, 1H), 7.28 (d, J = 8.4 Hz, 1H), 7.12 (dd, J = 8.4 Hz, 1.2 Hz, 1H), 7.04 (d, J = 7.6 Hz, 1H), 6.85 (d, J = 8.8 Hz, 1H); 6.83 (s, 1H), 6.71 (s, 1H), 5.65 (s, 1H), 4.90 (t, J = 7.2 Hz, 2H), 3.95 (s, 2H), 3.48 (d, J = 5.6 Hz, 2H), 2.34 (s, 3H), 2.18 (s, 3H), 2.14 (s, 3H), 2.13 (s, 3H); 1.63-1.55 (m, 2H), 1.43-1.35 (m, 1H), 1.14-0.96 (m, 2H), 0.72 (t, J = 6.4 Hz, 6H). | LCMS-A024: 471.3 (fragment); Rt = 1.825 min. |
| | 2-(2-((2-amino-2-oxoethoxy)(3,5-dimethylisoxazol-4- | | |
| | yl)methyl)benzofuran-5-yl)-N-(1-(2,4-dimethylphenyl)-4- | | |
| | methylpentyl)acetamide | | |
| 180 | 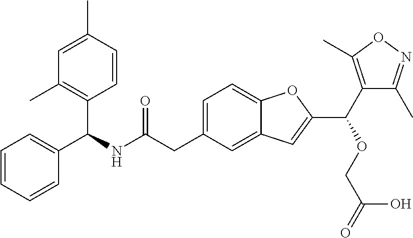 [SMILES: Cc1ccc([C@@H](NC(=O)Cc2ccc3oc([C@@H](OCC(=O)O)c4c(C)noc4C)cc3c2)c2ccccc2)c(C)c1] | ¹H NMR (DMSO, 500 MHz): δ 8.89 (d, J = 8.0 Hz, 1H), 7.50-7.16 (m, 8H), 6.98-6.88 (m, 4H), 6.18 (d, J = 8.0 Hz, 1H), 5.84 (s, 1H), 4.05 (s, 2H), 3.59 (s, 2H), 2.40 (s, 3H), 2.23 (s, 3H), 2.16 (s, 3H), 2.14 (s, 3H). | LC-MS012: 553.2 [M + H]⁺; Rt = 1.367 min |
| | 2-((S)-(3,5-dimethylisoxazol-4-yl)(5-(2-(((S)-(2,4- | | |
| | dimethylphenyl)(phenyl)methyl)amino)-2-oxoethyl)benzofuran- | | |
| | 2-yl)methoxy)acetic acid | | |

Following essential the same procedure as described in example 12, the compounds in table 9 were prepared.

TABLE 9

| | Name | NMR | LCMS |
|---|---|---|---|
| 181 | 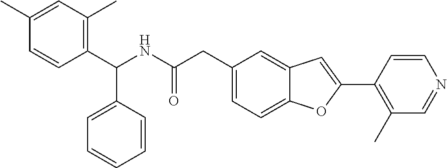 [SMILES: Cc1ccc(C(NC(=O)Cc2ccc3oc(-c4ccncc4C)cc3c2)c2ccccc2)c(C)c1] | 1H NMR (DMSO-d₆, 400 MHz): δ 8.91 (d, J = 8.8 Hz, 1H), 8.57 (s, 1H), 8.53 (d, J = 5.2 Hz, 1H), 7.80 (d, J = 5.2 Hz, 1H), 7.60 (d, J = 9.6 Hz, 2H), 7.50 (s, 1H), 7.31 (t, J = 7.6 Hz, 3H), 7.24 (t, J = 7.2 Hz, 1H), 7.18 (d, J = 7.6 Hz, 2H), 7.01- 6.95 (m, 3H), 6.20 (d, J = 8.0 Hz, 1H), 3.64 (s, 2H), 2.58 (s, 3H), 2.23 (s, 3H), 2.16 (s, 3H). | LCMS-012: 461.70 [M + H]⁺; Rt: 1.89 min. |
| | N-((2,4-dimethylphenyl)(phenyl)methyl)- | | |
| | 2-(2-(3-methylpyridin-4-yl)benzofuran-5-yl)acetamide | | |

Following essential the same procedure as described in example 35, the compounds in table 10 were prepared.

TABLE 10

| | Name | NMR | LCMS |
|---|---|---|---|
| 182 | 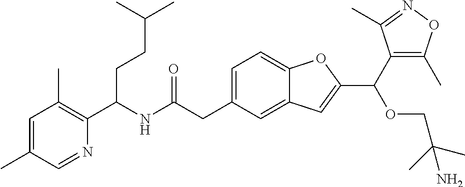 [SMILES: Cc1cnc(C(CCC(C)C)NC(=O)Cc2ccc3oc(C(OCC(C)(C)N)c4c(C)noc4C)cc3c2)c(C)c1] | ¹H NMR (CDCl₃, 400 MHz): δ 9.14 (d, J = 4.8 Hz, 1H), 8.35 (s, 1H), 7.95 (br, 2H), 7.86 (s, 1H); 7.39 (s, 1H), 7.30 (s, 1H), 7.10 (dd, J = 6.8 Hz, 14.8 Hz, 1H), 6.62 (s, 1H), 5.45 (s, 1H), 5.19 (s, 1H), 3.63-3.49 (m, 4H), 2.60 (s, 3H), 2.44 (s, 3H), 2.40 (s, 3H), 2.23(s, 3H), 1.97 (s, 1H), 1.76 (s, 1H), 1.53-1.49 (m, 1H), 1.47 (m, 1H), 1.37-1.25 (m, 5H), 1.06 (s, 1H), 1.26-1.03 (m, 1H), 0.84-0.80 (m, | LCMS-A024: 561.4 [M + H]⁺; Rt: 1.49 min |
| | 2-(2-((2-amino-2-methylpropoxy)(3,5- | 6H). | |
| | dimethylisoxazol-4-yl)methyl)benzofuran-5-yl)-N- | | |
| | (1-(3,5-dimethylpyridin-2-yl)-4-methylpentyl)acetamide | | |

Following essential the same procedure as described in example 36, the compounds in table 11 were prepared.

TABLE 11

| | Name | NMR | LCMS |
|---|---|---|---|
| 183 | 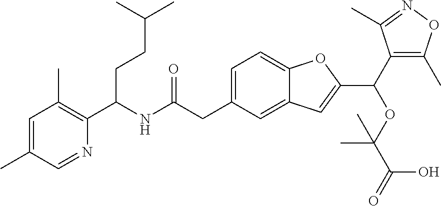 | ¹H NMR (DMSO-d₆, 400 MHz): δ 8.31 (d, J = 8.4 Hz, 1H); 8.21 (s, 1H), 7.41 (s, 1H); 7.37 (d, J = 8.4 Hz, 1H); 7.33 (s. 1H) 7.12 (d, J = 8.4 Hz, 1H), 6.74 (s, 1H), 5.89 (s, 1H), 5.00 (q, J = 7.6 Hz, 1H), 3.53- 3.40 (m, 2H), 2.40 (s, 3H), 2.24 (s, 3H), 2.28 (s, 3H), 2.15 (s, 3H), 1.98-1.62 (m, 2H), 1.45-1.38 (m, 1H), 1.37 (s, 3H) 1.33 (s, 3H), 1.13-1.01 (m, 1H), 0.99-0.91 (m, 1H), 0.75 (d, J = 6.4 Hz, 3H), 0.73 (d, J = 6.4 Hz, 3H). | LCMS-A024: 576.7 [M − H]⁺; Rt: 1.51 min |
| | 2-((3,5-dimethylisoxazol-4-yl)(5-(2-((1-(3,5-dimethylpyridin- | | |
| | 2-yl)-4-methylpentyl)amino)-2-oxoethyl)benzofuran- | | |
| | 2-yl)methoxy)-2-methylpropanoic acid | | |
| 184 | 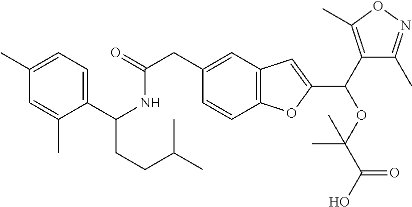 | ¹H NMR (MeOH-d₄, 400 MHz): δ 7.34 (s, 1H), 7.23 (d, J = 8.4 Hz, 1H), 7.07 (dd, J = 1.8 Hz, 8.6 Hz, 1H), 7.03 (d, J = 7.6 Hz, 1H), 6.86-6.83 (m, 2H), 6.56 (s, 1H), 5.73 (s, 1H), 4.90 (t, J = 7.4 Hz, 1H), 3.49 (d, J = 10.4 Hz, 1H), 3.42 (d, J = 10.4 Hz, 1H), 2.34 (s, 3H), 2.18 (s, 3H), 2.13 (s, 3H), 2.12 (s, 3H), 1.62-1.56 (m, 2H), 1.42 (s, 3H), 1.37 (s, 3H), 1.20- 0.99 (m, 3H), 0.72 (d, J = 6.4 Hz, 6H). | LCMS-A024: 575.0 [M + H]⁺; Rt = 1.930 min. |
| | 2-((3,5-dimethylisoxazol-4-yl)(5-(2-((1-(2,4-dimethylphenyl)- | | |
| | 4-methylpentyl)amino)-2-oxoethyl)benzofuran-2- | | |
| | yl)methoxy)-2-methylpropanoic acid | | |
| 185 | 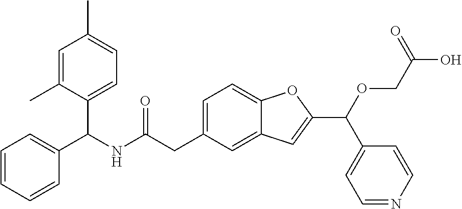 [SMILES: Cc1ccc(C(NC(=O)Cc2ccc3oc(C(OCC(=O)O)c4ccncc4)cc3c2)c2ccccc2)c(C)c1] | ¹H NMR (MeOH-d₄, 400 MHz): δ 8.54 (d, J = 5.2 Hz, 2H), 7.63 (d, J = 5.4 Hz, 2H), 7.52 (s, 1H), 7.37 (d, J = 8.4 Hz, 1H), 7.30-7.23 (m, 4H), 7.14 (d, J = 6.8 Hz, 2H), 6.98 (s, 1H), 6.94-6.92 (m, 2H), 6.87 (s, 1H), 6.28 (s, 1H), 5.90 (s, 1H), 4.07 (dd, J = 15.6, 1.04 Hz, 2H), 3.66 (s, 2H), 2.27 (s, 3H), 2.16 (s, 3H). | LCMS-A012: 535.00 [M + H]⁺; Rt = 1.50 min. |
| | 2-((5-(2-(((2,4-dimethylphenyl)(phenyl)methyl)amino)-2- | | |
| | oxoethyl)benzofuran-2-yl)(pyridin-4-yl)methoxy)acetic acid | | |
| 186 | 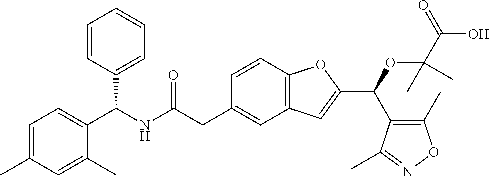 [SMILES: Cc1ccc([C@@H](NC(=O)Cc2ccc3oc([C@@H](OC(C)(C)C(=O)O)c4c(C)noc4C)cc3c2)c2ccccc2)c(C)c1] | ¹H NMR (MeOD, 400 MHz): δ 7.49 (s, 1H), 7.34 (d, J = 6.4 Hz, 1H), 7.30-7.21 (m, 4H), 7.14 (d, J = 6.0 Hz, 2H), 6.99 (s, 1H), 6.96- 6.92 (m, 2H), 6.70 (s, 1H), 6.29 (s, 1H) 5.86 (s, 1H), 3.66 (s, 2H), 2.47 (s, 3H), 2.28 (s, 3H), 2.26 (s, 3H), 2.17 (s, 3H), 1.52 (s, 3H), 1.48 (s, 3H). | LC-MSA026: 581.2 [M + H]⁺; Rt = 1.73min |
| | 2-((S)-(3,5-dimethylisoxazol-4-yl)(5-(2-(((S)-(2,4- | | |
| | dimethylphenyl)(phenyl)methyl)amino)-2-oxoethyl)benzofuran- | | |
| | 2-yl)methoxy)-2-methylpropanoic acid | | |
| 187 | 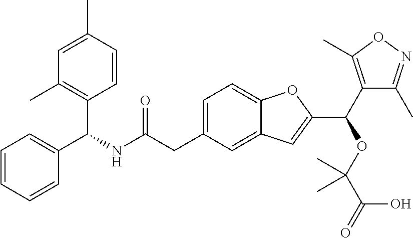 [SMILES: Cc1ccc([C@H](NC(=O)Cc2ccc3oc([C@H](OC(C)(C)C(=O)O)c4c(C)noc4C)cc3c2)c2ccccc2)c(C)c1] | ¹H NMR (DMSO, 400 MHz): δ 8.87 (d, J = 8.4 Hz, 1H), 7.46-7.15 (m, 8H), 6.99-6.95 (m, 3H), 6.78 (s, 1H), 6.17 (d, J = 8.4 Hz, 1H), 5.86 (s, 1H), 3.57 (s, 2H), 2.42 (s, 3H), 2.22 (s, 3H), 2.17 (s, 3H), 2.14 (s, 3H), 1.41 (s, 3H), 1.38 (s, 3H). | LC-MS012: 581.2 [M + H}⁺; Rt = 1.39 min. |
| | 2-((R)-(3,5-dimethylisoxazol-4-yl)(5-(2-(((R)-(2,4- | | |
| | dimethylphenyl)(phenyl)methyl)amino)-2-oxoethyl)benzofuran- | | |
| | 2-yl)methoxy)-2-methylpropanoic acid | | |
| 188 | 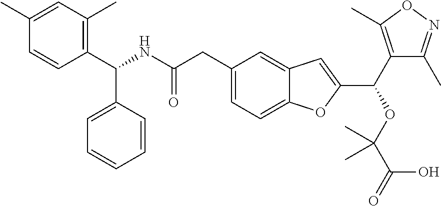 | ¹H NMR (DMSO, 400 MHz): δ 8.87 (d, J = 8.0 Hz, 1H), 7.45-7.14 (m, 8H), 6.98-6.92 (m, 3H), 6.76 (s, 1H), 6.16 (d, J = 8.4 Hz, 1H), 5.87 (s, 1H), 3.56 (s, 2H), 2.41 (s, 3H), 2.21 (s, 3H), 2.16 (s, 3H), 2.13 (s, 3H), 1.39 (s, 3H), 1.36 (s, 3H). | LC-MS012: 581.2 [M + H]⁺; Rt = 1.39 min. |
| | 2-((S)-(3,5-dimethylisoxazol-4-yl)(5-(2-(((R)-(2,4- | | |
| | dimethylphenyl)(phenyl)methyl)amino)-2-oxoethyl)benzofuran- | | |
| | 2-yl)methoxy)-2-methylpropanoic acid | | |
| 189 | 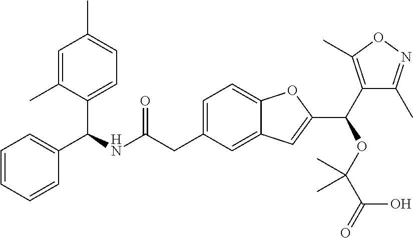 [SMILES: Cc1ccc([C@@H](NC(=O)Cc2ccc3oc([C@H](OC(C)(C)C(=O)O)c4c(C)noc4C)cc3c2)c2ccccc2)c(C)c1] | ¹H NMR (DMSO, 400 MHz): δ 12.67 (s, 1H), 8.86 (d, J = 8.4 Hz, 1H), 7.45-7.04 (m, 2H), 7.31-7.14 (m, 6H), 6.98-6.92 (m, 3H), 6.78 (s, 1H), 6.17 (d, J = 8.0 Hz, 1H), 5.84 (s, 1H), 3.56 (s, 2H), 2.41 (s, 3H), 2.22 (s, 3H), 2.16 (s, 3H), 2.13 (s, 3H), 1.41 (s, 3H), 1.38 (s, 3H). | LC-MS022: 581.0 [M + H]⁺; Rt = 1.818 min. |
| | 2-((R)-(3,5-dimethylisoxazol-4-yl)(5-(2-(((S)-(2,4- | | |
| | dimethylphenyl)(phenyl)methyl)amino)-2-oxoethyl)benzofuran- | | |
| | 2-yl)methoxy)-2-methylpropanoic acid | | |
| 190 | 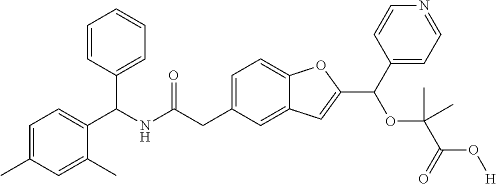 [SMILES: Cc1ccc(C(NC(=O)Cc2ccc3oc(C(OC(C)(C)C(=O)O)c4ccncc4)cc3c2)c2ccccc2)c(C)c1] | ¹H NMR (MeOH, 400 MHz): δ 8.41 (s, 2H), 7.50 (d, J = 5.6 Hz, 2H), 7.38 (s, 1H), 7.23 (d, J = 8.4 Hz, 1H), 7.17-7.09 (m, 5H), 7.02 (d, J = 5.6 Hz, 2H), 6.87-6.82 (m, 3H), 6.01 (s, 1H), 6.17 (s, 1H), 3.54 (s, 2H), 2.16 (s, 3H), 2.05 (s, 3H), 1.41 (s, 3H), 1.40 (s, 3H). | LC-MS A026 m/z 563 [M + H]⁺; Rt = 1.51 min |
| | 2-((5-(2-(((2,4-dimethylphenyl)(phenyl)methyl) | | |
| | amino)-2-oxoethyl)benzofuran-2-yl)(pyridin-4-yl)methoxy)-2- | | |
| | methylpropanoic acid | | |

Following essential the same procedure as described in example 37, the compounds in table 12 were prepared.

TABLE 12

| | Name | NMR | LCMS |
|---|---|---|---|
| 191 | 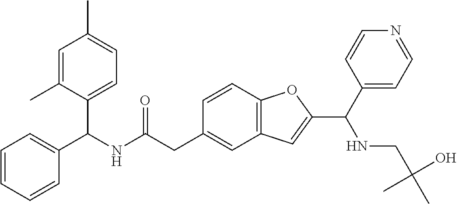 [SMILES: Cc1ccc(C(NC(=O)Cc2ccc3oc(C(NCC(C)(C)O)c4ccncc4)cc3c2)c2ccccc2)c(C)c1] | ¹H NMR (MeOH-d₄, 400 MHz): δ 8.52 (d, J = 5.6 Hz, 2H), 7.60 (d, J = 6.0 Hz, 2H)), 7.49 (s, 1H), 7.34 (d, J = 8.4 Hz, 1H), 7.30-7.20 (m, 4H), 7.14-7.13 (m, 2H), 6.98 (s, 1H), 6.94-6.93 (m, 2H), 6.70 (s, 1H), 6.28 (s, 1H), 5.08 (s, 1H), 3.65 (s, 2H), 2.64-2.50 (m, 2H), 2.27 (s, 3H), 2.16 (s, 3H), 1.25 (s, 3H), 1.23 (s, 3H). | LCMS-A012: 548.0 [M + H]⁺; Rt = 1.79 min. |
| | N-((2,4-dimethylphenyl)(phenyl)methyl)- | | |
| | 2-(2-(((2-hydroxy-2-methylpropyl)amino)(pyridin-4- | | |
| | yl)methyl)benzofuran-5-yl)acetamide | | |

Following essential the same procedure as described in example 38, the compounds in table 13 were prepared.

TABLE 13

| | Name | NMR | LCMS |
|---|---|---|---|
| 192 | 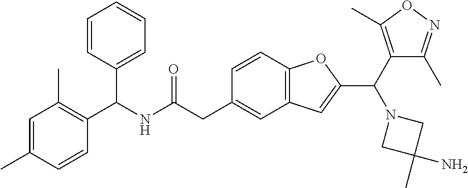 | ¹H NMR (MeOH-d₄, 400 MHz): δ 7.49 (s, 1H), 7.35 (d, J = 8.8 Hz, 1H), 7.30-7.20 (m, 4H), 7.15-7.13 (m, 2H), 7.68-6.93 (m, 3H), 6.67 (s, 1H), 6.28 (s, 1H), 4.73 (s, 1H) 3.66 (s, 2H), 3.36-3.25 (m, 4H), 2.55 (s, 3H), 2.31 (s, 3H), 2.27 (s, 3H), 2.16 (s, 3H), 1.58 (s, 3H). | LCMS-A010: no mass [M + H]⁺, fragment at 477.2, Rt = 1.51 min. |
| | 2-(2-((3-amino-3-methylazetidin-1-yl)(3,5- | | |
| | dimethylisoxazol-4-yl)methyl)benzofuran-5-yl)-N- | | |
| | ((2,4-dimethylphenyl)(phenyl)methyl)acetamide | | |
| 193 | 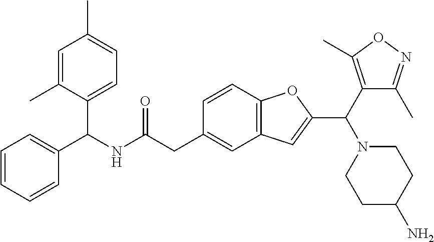 [SMILES: Cc1ccc(C(NC(=O)Cc2ccc3oc(C(c4c(C)noc4C)N4CCC(N)CC4)cc3c2)c2ccccc2)c(C)c1] | ¹H NMR (CDCl₃, 400 MHz): δ 8.88 (d, J = 8.4 Hz, 2H), 7.44-7.41 (m, 2H), 7.31-7.22 (m, 3H), 7.16- 7.14 (m, 3H), 6.98-6.92 (m, 3H), 6.85 (s, 1H), 6.16 (d, J = 8.0 Hz, 1H), 4.68 (s, 1H), 3.56 (s, 1H), 2.82-2.66 (m, 2H), 2.39 (s, 3H), 2.21 (s, 3H), 2.20 (s, 3H), 2.10 (s, 3H), 2.05-1.96 (m, 4H), 1.25-1.22 (m, 2H). | LCMS-A012: no mass [M + H]⁺; fragment at 477, Rt = 1.95 min. |
| | 2-(2-((4-aminopiperidin-1-yl)(3,5-dimethylisoxazol-4- | | |
| | yl)methyl)benzofuran-5-yl)-N-((2,4- | | |
| | dimethylphenyl)(phenyl)methyl)acetamide | | |
| 194 | 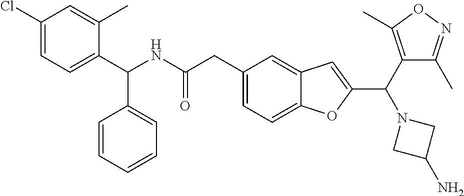 | ¹H NMR (MeOD, 400 MHz): δ 7.48 (s, 1H), 7.36-7.26 (m, 4H), 7.22-7.05 (m, 6H), 6.66 (s, 1H), 6.28 (s, 1H), 4.62 (s, 1H), 3.73 (t, J = 7.0 Hz, 1H), 3.66 (s, 2H), 3.59 (t, J = 6.8 Hz, 1H), 3.48 (t, J = 7.2 Hz, 1H), 2.93 (t, J = 7.2 Hz, 1H), 2.84 (t, J = 7.2 Hz, 1H), 2.54 (s, 3H), 2.29 (s, 3H), 2.19 (s, 3H). | LCMSA036: no mass [M + H]⁺; Rt: 1.34 min |
| | 2-(2-((3-aminoazetidin-1-yl)(3,5-dimethylisoxazol-4- | | |
| | yl)methyl)benzofuran-5-yl)-N-((4-chloro-2- | | |
| | methylphenyl)(phenyl)methyl)acetamide | | |

Following essential the same procedure as described in example 39, the compounds in table 14 were prepared.

TABLE 14

| | Name | NMR | LCMS |
|---|---|---|---|
| 195 | 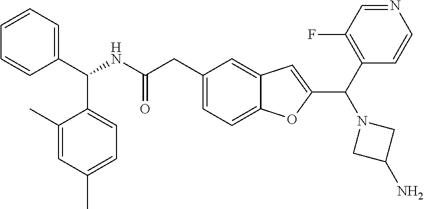 [SMILES: Cc1ccc([C@@H](NC(=O)Cc2ccc3oc(C(c4ccncc4F)N4CC(N)C4)cc3c2)c2ccccc2)c(C)c1] | ¹H NMR (MeOH-d₄, 400 MHz): δ 8.52-8.49 (m, 2H), 7.87-7.84 (m, 1H), 7.51 (d, J = 1.2 Hz, 1H), 7.36 (d, J = 8.8 Hz, 1H), 7.31-7.23 (m, 5H), 7.14 (d, J = 7.2 Hz, 2H), 6.99-6.92 (m, 4H), 6.81 (s, 1H), 6.29 (s, 1H), 5.26 (s, 1H), 3.99- 3.93 (m, 1H), 3.76-3.67 (s, 5H), 3.45-3.42 (m, 1H), 2.28 (s, 3H), 2.17 (s, 3H). | LCMS-036: 549.2 [M + H]⁺; Rt = 1.420 min. |
| | 2-(2-((3-aminoazetidin-1-yl)(3-fluoropyridin-4- | | |
| | yl)methyl)benzofuran-5-yl)-N-((S)-(2,4- | | |
| | dimethylphenyl)(phenyl)methyl)acetamide | | |
| 196 | 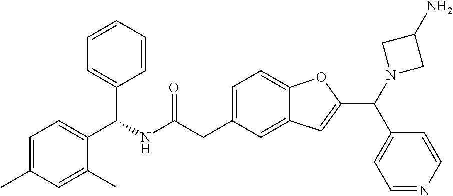 [SMILES: Cc1ccc([C@@H](NC(=O)Cc2ccc3oc(C(c4ccncc4)N4CC(N)C4)cc3c2)c2ccccc2)c(C)c1] | ¹H NMR (MeOD, 400 MHz): δ 8.43 (d, J = 5.6 Hz, 2H), 7.48 (d, J = 7.0 Hz, 2H), 7.38 (s,1H), 7.24- 7.01 (m, 7H), 6.87-6.81 (m, 3H), 6.63 (s, 1H), 6.17 (s, 1H), 4.68 (s, 1H), 4.52 (s, 1H), 3.69-3.48 (m, 5H), 3.21-3.01 (m, 4H), 2.15 (s, 3H), 2.05 (s, 3H). | LC-MS (024): 531.2 [M + H]⁺; Rt: 1.34 min |
| | 2-(2-((3-aminoazetidin-1-yl)(pyridin-4- | | |
| | yl)methyl)benzofuran-5-yl)-N-((2,4- | | |
| | dimethylphenyl)(phenyl)methyl)acetamide | | |
| 197 | 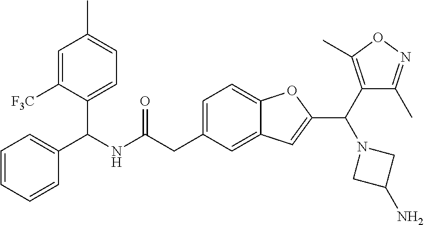 | ¹H NMR (MeOD, 400 MHz): δ 7.54 (s, 1H), 7.48 (s, 1H), 7.39- 7.19 (m, 7H), 7.10 (d, J = 6.8 Hz, 2H), 6.67 (s, 1H), 6.56 (s, 1H), 4.67 (s, 1H), 3.76-3.61 (m, 4H), 3.54 (t, J = 3.4 Hz, 3H), 3.10-3.04 (m, 2H), 2.55 (s, 3H), 2.41 (s, 3H), 2.30 (s, 3H). | LCMSA039: no mass [M + H]⁺; Rt: 1.71 min |
| | 2-(2-((3-aminoazetidin-1-yl)(3,5-dimethylisoxazol- | | |
| | 4-yl)methyl)benzofuran-5-yl)-N-((4-methyl-2- | | |
| | (trifluoromethyl)phenyl)(phenyl)methyl)acetamide | | |
| 198 | 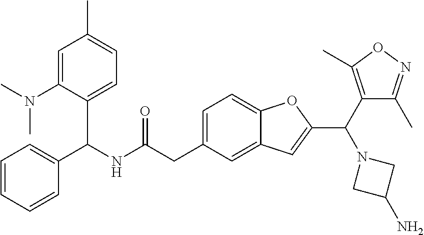 [SMILES: Cc1ccc(C(NC(=O)Cc2ccc3oc(C(c4c(C)noc4C)N4CC(N)C4)cc3c2)c2ccccc2)c(N(C)C)c1] | ¹H NMR (MeOD, 400 MHz): δ 7.50 (s, 1H), 7.37 (d, J = 8.8 Hz, 1H), 7.30-7.13 (m, 8H), 7.03 (d, J = 7.6 Hz, 1H), 6.69 (s, 1H), 6.59 (s, 1H), 4.74 (s, 1H), 4.00-3.93 (m, 1H), 3.70-3.66 (m, 3H), 3.60 (t, J = 7.6 Hz, 1H), 3.28 (t, J = 4.8 Hz, 2H), 2.67 (s, 6H), 2.56 (s, 3H), 2.34 (s, 3H), 2.31 (s, 3H). | LCMSA020: 578 [M + H]⁺; Rt: 1.68 min |
| | 2-(2-((3-aminoazetidin-1-yl)(3,5-dimethylisoxazol-4- | | |
| | yl)methyl)benzofuran-5-yl)-N-((2-(dimethylamino)- | | |
| | 4-methylphenyl)(phenyl)methyl)acetamide | | |
| 199 | 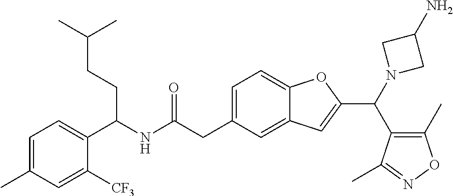 | ¹H NMR (MeOD, 400 MHz): δ 7.48 (s, 3H), 7.39 (d, J = 8.4 Hz, 1H), 7.34 (d, J = 8.4 Hz, 1H), 7.18 (d, J = 8.4 Hz, 1H), 6.65 (s, 1H), 5.20 (t, J = 7.2 Hz, 1H), 4.61 (s, 1H), 3.71 (t, J = 7.2 Hz, 1H), 3.64- 3.57 (m, 3H), 3.48 (t, J = 6.8 Hz, 1H), 2.92 (t, J = 7.2 Hz, 1H), 2.83 (t, J = 6.8 Hz, 1H), 2.53 (s, 3H), 2.39 (s, 3H), 2.29 (s, 3H), 1.71- 1.65 (m, 2H), 1.51-1.43 (m, 1H), 1.31-1.08 (m, 2H), 0.82 (d, J = 6.8 Hz, 3H), 0.80 (d, J = 6.8 Hz, | LCMSA036: no mass [M + H]⁺; Rt: 1.42 min |
| | 2-(2-((3-aminoazetidin-1-yl)(3,5-dimethylisoxazol-4- | 3H). | |
| | yl)methyl)benzofuran-5-yl)-N-(4-methyl-1-(4-methyl- | | |
| | 2-(trifluoromethyl)phenyl)pentyl)acetamide | | |
| 200 | 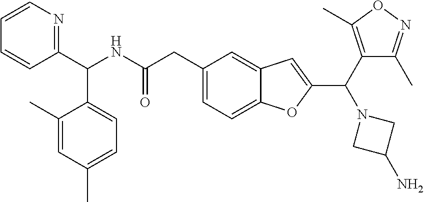 [SMILES: Cc1ccc(C(NC(=O)Cc2ccc3oc(C(c4c(C)noc4C)N4CC(N)C4)cc3c2)c2ccccn2)c(C)c1] | ¹H NMR (MeOD, 400 MHz): δ 8.47 (d, J = 3.2 Hz, 1H), 7.92 (t, J = 7.6 Hz, 1H), 7.42-6.80 (m, 7H), 6.59-6.29 (m, 3H), 4.67 (s, 2H), 3.85-3.12 (m, 7H), 2.44 (s, 3H), 2.20 (s, 3H), 2.16 (s, 6H). | LC-MS (036): 550.2 [M + H]⁺; Rt: 1.22 min |
| | 2-(2-((3-aminoazetidin-1-yl)(3,5-dimethylisoxazol-4- | | |
| | yl)methyl)benzofuran-5-yl)-N-((2,4- | | |
| | dimethylphenyl)(pyridin-2-yl)methyl)acetamide | | |
| 201 | 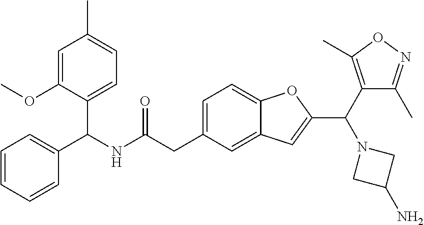 | ¹H NMR (DMSO, 400 MHz): δ 8.75 (d, J = 8.8 Hz, 1H), 7.46-7.21 (s, 1H), 7.41 (d, J = 8.8 Hz, 1H), 7.26 (t, J = 7.2 Hz, 2H), 7.20-7.12 (m, 5H), 6.77 (d, J = 8.8 Hz, 2H), 6.72 (d, J = 6.8 Hz, 1H), 6.31 (t, J = 8.8 Hz, 1H), 4.70 (s, 1H), 3.76- 3.53 (m, 8H), 3.41-3.37 (m, 3H), 3.07-3.05 (m, 2H), 2.27 (s, 3H), 2.20 (s, 3H). | LC-MS (044): 493.2; Rt = 1.65 min, purity 97 % (214 nm). |
| | 2-(2-((3-aminoazetidin-1-yl)(3,5-dimethylisoxazol-4- | | |
| | yl)methyl)benzofuran-5-yl)-N-((2-methoxy-4- | | |
| | methylphenyl)(phenyl)methyl)acetamide | | |

Following essential the same procedure as described in example 1, the compounds in table 15 were prepared.

TABLE 15

| | Name | NMR | LCMS |
|---|---|---|---|
| 202 | 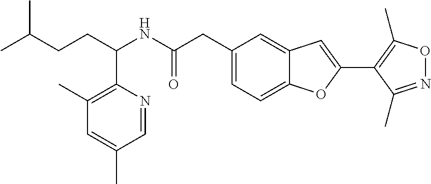 [SMILES: Cc1cnc(C(CCC(C)C)NC(=O)Cc2ccc3oc(-c4c(C)noc4C)cc3c2)c(C)c1] 2-(2-(3,5-dimethylisoxazol-4- yl)benzofuran-5-yl)-N-(1-(3,5- dimethylpyridin-2-yl)-4- methylpentyl)acetamide | ¹H NMR (CDCl₃, 400 MHz): δ 8.29 (s, 1H), 7.72 (s, 1H), 7.49 (d, J = 1.2 Hz, 1H), 7.40 (d, J = 8.4 Hz, 1H), 7.20 (dd, J= 8.8 Hz, J = 2 Hz, 1H), 6.53 (s, 1H), 5.20 (q, J = 7.6 Hz, 1H), 3.68 (d, J = 14.4 Hz, 1H), 3.61 (d, J = 14.4 Hz, 1H), 2.67 (s, 3H), 2.57 (s, 3H), 2.48 (s, 3H), 2.40 (s, 3H), 1.94-1.91 (m, 1H), 1.81-1.72 (m, 1H), 1.52-1.43 (m, 1H), 1.31-1.25 (m, 2H), 1.11- 1.02 (m, 1H), 0.82 (d, J = 7.2 Hz, 3H), 0.80 (d, J = 7.2 Hz, 3H). | LCMS-P1: 460.0 [M + H]⁺; Rt = 1.51 min. |
| 203 | 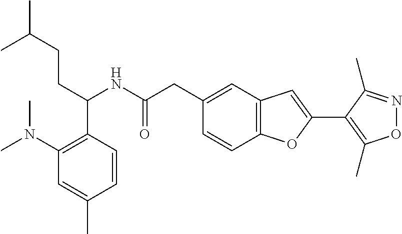 [SMILES: Cc1ccc(C(CCC(C)C)NC(=O)Cc2ccc3oc(-c4c(C)noc4C)cc3c2)c(N(C)C)c1] N-(1-(2-(dimethylamino)-4- methylphenyl)-4-methylpentyl)- 2-(2-(3,5-dimethylisoxazol-4- yl)benzofuran-5-yl)acetamide | ¹H NMR (DMSO-d₆, 400 MHz): δ 8.74 (br, 1H), 7.60-7.11 (m, 7H), 5.12 (s, 1H), 3.63 (d, J = 14 Hz, 1H), 3.56 (d, J = 14 Hz, 1H), 2.88 (br, 6H), 2.73 (s, 3H), 2.56 (s, 3H), 2.35 (s, 3H), 1.79-1.65 (m, 2H), 1.59-1.53 (m, 1H), 1.25-1.06 (m, 2H), 0.89-0.86 (m, 6H). | LCMS-A024: 488.7, [M + H]⁺; Rt = 1.48 min. |

Following essential the same procedure as described in example 40, the compounds in table 16 were prepared.

TABLE 16

| | Name | NMR | LCMS |
|---|---|---|---|
| 204 | 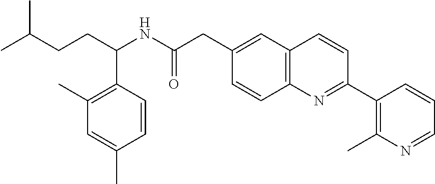 [SMILES: Cc1ccc(C(CCC(C)C)NC(=O)Cc2ccc3nc(-c4cccnc4C)ccc3c2)c(C)c1] N-(1-(2,4-dimethylphenyl)-4- methylpentyl)-2-(2-(2- metehylpyridin-3-yl)quinolin-6- yl)acetamide | ¹H NMR (DMSO-d₆, 400 MHz): δ 8.74 (s, 1H), 8.56 (d, J = 8.4 Hz, 1H), 8.47 (d, J = 8.4 Hz, 1H), 8.28 (d, J = 7.2 Hz, 1H), 7.99 (d, J = 8.8 Hz, 1H), 7.87 (s, 1H), 7.81 (d, J = 8.8 Hz, 1H), 7.73 (dd, J = 1.8 Hz, 8.6 Hz, 1H), 7.70-7.67 (m, 1H), 7.22 (d, J = 8.0 Hz, 1H), 6.97 (d, J = 8.4 Hz, 1H), 6.91 (s, 1H), 4.92- 4.86 (m, 1H), 3.72-3.63 (m, 2H), 2.68 (s, 3H), 2.25 (s, 3H), 2.21 (s, 3H), 1.65-1.56 (m, 2H), 1.51-1.44 (m, 1H), 0.85 (t, J = 6.8 Hz, 2H), 0.79 (t, J = 7.2 Hz, 6H). | LCMS-A024: 466.1 [M + H]⁺; Rt = 1.477 min. |

Following essential the same procedure as described in example 29, the compounds in table 17 were prepared.

TABLE 17

| Ex. | Name | NMR | LCMS |
|---|---|---|---|
| 205 | 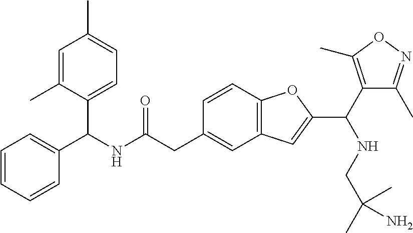 [SMILES: Cc1ccc(C(NC(=O)Cc2ccc3oc(C(NCC(C)(C)N)c4c(C)noc4C)cc3c2)c2ccccc2)c(C)c1] 2-(2-(((2-amino-2- methylpropyl)amino)(3,5- dimethylisoxazol-4-yl) methyl)benzofuran-5-yl)-N-((2,4- dimethylphenyl)(phenyl)methyl) acetamide | ¹H NMR (MeOH-d₄, 400 MHz): δ 7.50 (s, 1H), 7.37 (d, J = 8.4 Hz, 1H), 7.28-7.23 (m, 4H), 7.14 (d, J = 7.2 Hz, 2H), 6.98 (s, 1H), 6.93 (m, 2H), 6.75 (s, 1H), 6.28 (s, 1H), 5.00 (s, 1H), 3.67 (s, 2H), 2.69-2.68 (m, 2H), 2.40 (s, 3H), 2.27 (s, 3H), 2.26 (s, 3H), 2.17 (s, 3H), 1.25 (s, 6H). | LCMS- A024: 587.30 [M + Na]⁺; Rt = 1.46 min. |
| 206 | 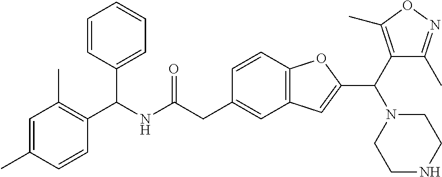 [SMILES: Cc1ccc(C(NC(=O)Cc2ccc3oc(C(c4c(C)noc4C)N4CCNCC4)cc3c2)c2ccccc2)c(C)c1] 2-(2-((3,5-dimethylisoxazol-4- yl)(piperazin-1- yl)methyl)benzofuran-5-yl)-N- ((2,4- dimethylphenyl)(phenyl)methyl) acetamide | ¹H NMR (CDCl₃, 400 MHz): δ 7.49 (s, 1H), 7.37 (d, J = 8.0 Hz, 1H), 7.28-7.21 (m, 4H), 7.13 (d, J = 6.8 Hz, 2H), 6.98 (s, 1H), 6.93 (m, 2H), 6.79 (s, 1H), 6.28 (s, 1H), 4.64 (s, 1H), 4.59 (s, 1H), 3.66 (s, 2H), 3.14 (t, 4H), 2.46-2.50 (m, 4H), 2.47 (s, 3H), 2.34 (s, 3H), 2.27 (s, 3H), 2.16 (s, 3H). | LCMS- A024: 563.5 [M + H]⁺; Rt = 1.45 min. |
| 207 | 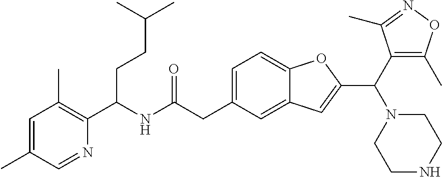 [SMILES: Cc1cnc(C(CCC(C)C)NC(=O)Cc2ccc3oc(C(c4c(C)noc4C)N4CCNCC4)cc3c2)c(C)c1] 2-(2-((3,5-dimethylisoxazol-4- yl)(piperazin-1- yl)methyl)benzofuran-5-yl)-N-(1- (3,5-dimethylpyridin-2-yl)-4- methylpentyl)acetamide] | ¹H NMR (CDCl₃, 400 MHz): δ 9.49 (s, 1H); 8.36 (s, 1H); 7.86 (s, 1H); 7.42 (s, 1H); 7.30 (q, J = 7.6 Hz, 1H); 7.15 (d, J = 8 Hz, 1H), 6.59 (s, 1H), 5.82 (s, 1H); 5.16 (d, J = 5.6 Hz, 1H), 4.45 (s, 1H), 3.62 (q, J = 14 Hz, 2H); 3.21 (br, 3H), 2.70 (br, 3H), 2.60 (s, 3H), 2.44 (s, 6H), 2.30 (s, 3H), 1.99 (br, 1H), 1.75 (br, 1H), 1.51-1.48 (m, 1H), 1.31-1.25 (m, 2H); 1.07-1.05 (m, 1H), 0.84- 0.78 (m, 6H). | LCMS- A024: 558.3 [M + H]⁺; Rt: 1.44 min |
| 208 | 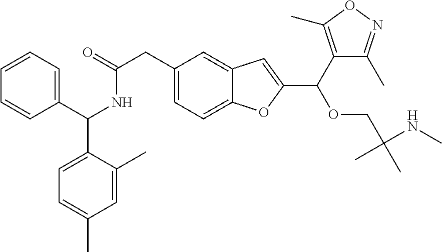 [SMILES: CNC(C)(C)COC(c1cc2cc(CC(=O)NC(c3ccccc3)c3ccc(C)cc3C)ccc2o1)c1c(C)noc1C] 2-(2-((3,5-dimethylisoxazol-4- yl)(2-methyl-2- (methylamino)propoxy)methyl) benzofuran-5-yl)-N-((2,4- dimethylphenyl)(phenyl)methyl) acetamide | ¹H NMR (CDCl₃, 400 MHz): δ 9.12 (br, 1H), 7.35-7.08 (m, 5H), 6.99- 6.64 (m, 7H), 6.27 (d, J = 8.0 Hz, 1H), 5.97 (d, J = 8.0 Hz, 1H), 5.43 (s, 1H), 3.59 (s, 2H), 3.56-3.45 (m, 2H), 2.57 (s, 3H), 2.31 (s, 3H), 2.20 (s, 3H), 2.15 (s, 3H), 2.12 (s, 3H), 1.21 (s, 6H). | LCMS- A024: 580.3 [M + H]⁺; Rt: 1.31 min |

Following essentially the procedure as described in Example 52, the compound in Table 18 was prepared.

TABLE 18

| Example | Structure/Name | NMR | LCMS |
|---|---|---|---|
| 209 | 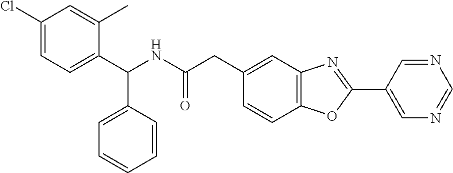 [SMILES: Cc1cc(Cl)ccc1C(NC(=O)Cc1ccc2oc(-c3cncnc3)nc2c1)c1ccccc1] N-((4-chloro-2- methylphenyl)(phenyl)methyl)- 2-(2-(pyrimidin-5- yl)benzo[d]oxazol-5- yl)acetamide | ¹H NMR (DMSO, 500 MHz): δ 9.52 (s, 1H), 9.42 (s, 1H), 9.04 (d, J = 8.5 Hz, 1H), 7.80-7.76 (m, 2H), 7.42- 7.12 (m, 10H), 6.21 (d, J = 8.0 Hz, 1H), 3.71 (s, 2H), 2.20 (s, 3H). | LC-MS: 469 [M + H]⁺; Rt: 1.680 min |
| 210 | 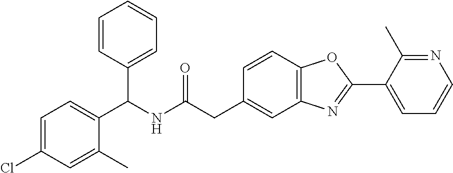 [SMILES: Cc1cc(Cl)ccc1C(NC(=O)Cc1ccc2oc(-c3cccnc3C)nc2c1)c1ccccc1] N-((4-chloro-2- methylphenyl)(phenyl)methyl)- 2-(2-(2-methylpyridin-3- yl)benzo[d]oxazol-5- yl)acetamide | ¹H NMR (DMSO, 500 MHz): δ 9.05 (d, J = 8.0 Hz, 1H), 8.69-8.48 (m, 2H), 7.77-7.14 (m, 14H), 6.29 (d, J = 8.5 Hz, 1H), 3.71 (s, 2H), 2.97 (s, 3H), 2.22 (s, 3H). | LC-MS: 482 [M + H]⁺; Rt: 1.604 min |
| 211 | 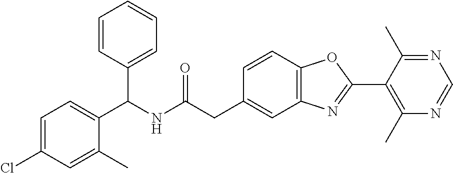 N-((4-chloro-2- methylphenyl)(phenyl)methyl)- 2-(2-(4,6-dimethylpyrimidin-5- yl)benzo[d]oxazol-5- yl)acetamide | ¹H NMR (DMSO, 500 MHz): δ 9.09-9.04 (m, 3H), 7.78-7.76 (m, 2H), 7.74-7.14 (m, 11H), 6.21 (d, J = 10.5, 1H), 3.71 (s, 2H), 2.54 (s, 3H), 2.50 (s, 3H), 2.21 (s, 3H). | LC-MS: 515 [M + H]⁺; Rt: 1.688 min |
| 212 | 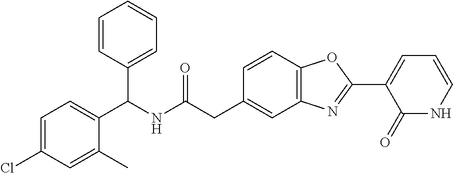 [SMILES: Cc1cc(Cl)ccc1C(NC(=O)Cc1ccc2oc(-c3ccc[nH]c3=O)nc2c1)c1ccccc1] N-((4-chloro-2- methylphenyl)(phenyl)methyl)- 2-(2-(2-oxo-1,2-dihydropyridin- 3-yl)benzo[d]oxazol-5- yl)acetamide | ¹H NMR (DMSO, 500 MHz): δ 12.59 (d, J = 5.0 Hz, 1H), 12.11 (s, 1H), 9.87 (s, 1H), 8.94 (d, J = 8.5 Hz, 1H), 8.49-8.38 (m, 2H), 7.78- 7.17 (m, 8H), 6.82-6.18 (m, 3H), 3.44 (s, 2H), 2.08 (s, 3H). | LC-MS: 484 [M + H]⁺; Rt: 1.551 min; |

Following essentially the procedure as described in Example 11, the compound in Table 19 was prepared.

TABLE 19

| Example | Structure/Name | NMR | LCMS |
|---|---|---|---|
| 213 | 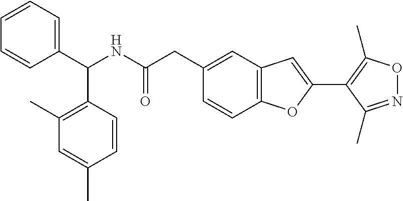 methyl 4-(5-(2-(((2,4- dimethylphenyl)(phenyl)methyl) amino)-2-oxoethyl)benzofuran- 2-yl)-5-methylisoxazole-3- carboxylate | ¹H NMR (CDCl₃, 400 MHz): δ 7.50 (d, J = 1.2 Hz, 1H), 7.46 (d, J = 8.8 Hz, 1H), 7.25 (s, 1H), 7.23 (s, 2H), 7.21 (s, 1H), 7.06 (d, J = 6.4 Hz, 2H), 6.97 (s, 1H), 6.90 (d, J = 7.6 Hz, 1H), 6.75 (d, J = 8.0 Hz, 1H), 6.38 (d, J = 8.4 Hz, 1H), 5.63 (d, J = 7.6 Hz, 1H), 4.02 (s, 3H), 3.73 (s, 2H), 2.81 (s, 3H), 2.28 (s, 3H), 2.21 (s, 3H). | LCMSA026: 509.2 [M + H]⁺; Rt = 2.101 min |

Following essentially the procedure as described in Example 43, the compound in Table 20 was prepared.

TABLE 20

| Example | Structure/Name | NMR | LCMS |
|---|---|---|---|
| 214 | 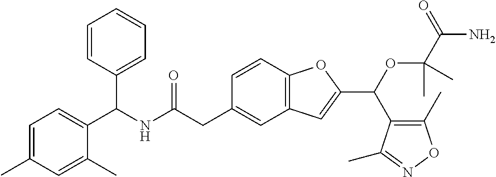 2-((3,5-dimethylisoxazol-4- yl)(5-(2-(((2,4- dimethylphenyl)(phenyl)methyl) amino)-2-oxoethyl)benzofuran- 2-yl)methoxy)-2- methylpropanamide | ¹H NMR (DMSO, 400 MHz): δ 8.86 (d, J = 8.4 Hz, 1H), 7.45-7.41 (m, 2H), 7.31-7.14 (m, 7H), 6.98-6.94 (m, 4H), 6.83 (s, 1H), 6.16 (d, J = 8.4 Hz, 1H), 5.82 (s, 1H), 3.57 (s, 2H), 2.44 (s, 3H), 2.22 (s, 3H), 2.18 (s, 3H), 2.13 (s, 3H), 1.33 (s, 3H), 1.30 (s, 3H). | LC-MS (A026): No mass [M + H]⁺. Rt = 1.91 min |
| 215 | 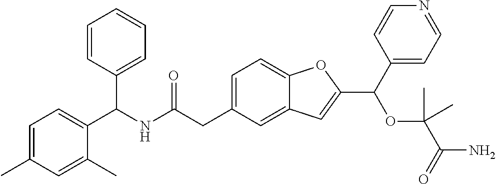 [SMILES: Cc1ccc(C(NC(=O)Cc2ccc3oc(C(OC(C)(C)C(N)=O)c4ccncc4)cc3c2)c2ccccc2)c(C)c1] 2-((5-(2-(((2,4- dimethylphenyl)(phenyl)methyl) amino)-2-oxoethyl)benzofuran- 2-yl)(pyridin-4-yl)methoxy)-2- methylpropanamide | ¹H NMR (MeOH, 400 MHz): δ 8.68 (s, 2H), 7.55 (d, J = 4.8 Hz, 2H), 7.37 (s, 1H), 7.26 (d, J = 8.4 Hz, 1H), 7.17-7.11 (m, 4H), 7.02 (d, J = 6.8 Hz, 2H), 6.87-6.80 (m, 3H), 6.54 (s, 1H), 6.16-6.15 (m, 1H), 5.94 (s, 1H), 3.54 (s, 2H), 2.16 (s, 3H), 2.05 (s, 5H), 1.38 (s, 3H), 1.32 (s, 3H). | LC-MS A012 m/z 562.3 [M + H]⁺; Rt = 1.38 min |

Following essentially the procedure as described in Example 46, the compound in Table 21 was prepared.

TABLE 21

| Example | Structure/Name | NMR | LCMS |
|---|---|---|---|
| 216 | 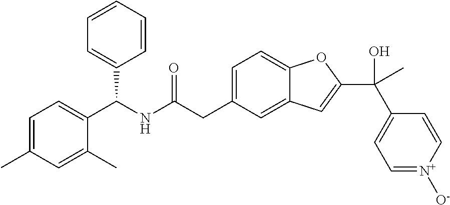 [SMILES: Cc1ccc([C@@H](NC(=O)Cc2ccc3oc(C(C)(O)c4cc[n+]([O-])cc4)cc3c2)c2ccccc2)c(C)c1] 4-(1-(5-(2-(((S)-(2,4- dimethylphenyl)(phenyl)methyl) amino)-2-oxoethyl)benzofuran- 2-yl)-1-hydroxyethyl)pyridine 1- oxide | ¹H NMR (CDCL3, 400 MHz): δ 8.19 (d, J = 6.0 Hz, 2H), 7.44-7.06 (m, 9H), 6.97-6.66 (m, 6H), 6.37 (d, J = 8.0 Hz, 1H), 5.99 (d, J = 7.6 Hz, 1H), 3.69 (s, 2H), 2.28 (s, 3H), 2.21 (s, 3H), 1.95 (s, 3H). | LC-MS (020): 507.2 [M + H]⁺; Rt: 1.57 min |
| 217 | 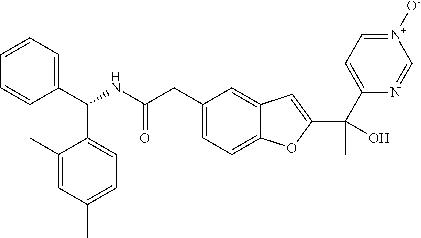 [SMILES: Cc1ccc([C@@H](NC(=O)Cc2ccc3oc(C(C)(O)c4cc[n+]([O-])cn4)cc3c2)c2ccccc2)c(C)c1] 4-(1-(5-(2-(((S)-(2,4- dimethylphenyl)(phenyl)methyl) amino)-2-oxoethyl)benzofuran- 2-yl)-1-hydroxyethyl)pyrimidine 1-oxide | ¹H NMR (CD₃Cl, 400 MHz): δ 8.96 (s, 1H), 8.39 (s, 1H), 7.65 (br, 1H), 7.45 (s, 1H), 7.37 (d, J = 8.0 Hz, 1H), 7.25-7.16 (m, 3H), 7.04 (d, J = 6.4 Hz, 2H), 6.97 (s, 1H), 6.89 (d, J = 8.0 Hz, 1H), 6.74 (d, J = 10.4 Hz, 2H), 6.36 (d, J = 8.4 Hz, 1H), 5.91 (d, J = 8.4 Hz, 1H), 3.70 (s, 2H), 2.28 (s, 3H), 2.20 (s, 3H), 2.01 (s, 3H). | LCMSA038: 508.2 [M + H]⁺; Rt = 1.645 min |
| 218 | 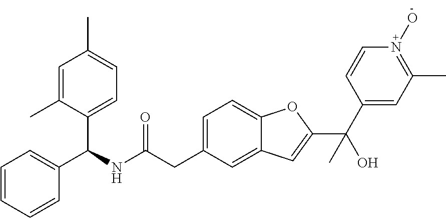 4-(1-(5-(2-(((S)-(2,4- dimethylphenyl)(phenyl)methyl) amino)-2-oxoethyl)benzofuran- 2-yl)-1-hydroxyethyl)-2- methylpyridine 1-oxide | ¹H NMR (MeOD, 400 MHz): δ 8.82 (d, J = 8.0 Hz, 1H), 8.31 (d, J = 6.8 Hz, 1H), 7.68 (d, J = 2.8 Hz, 1H), 7.55-7.53 (m, 2H), 7.38-7.23 (m, 5H), 7.16 (s, 1H), 7.14 (s, 1H), 7.00-6.95 (m, 3H), 6.78 (s, 1H), 6.29 (d, J = 2.8 Hz, 1H), 3.67 (s, 2H), 2.54 (s, 3H), 2.29 (s, 3H), 2.18 (s, 3H), 1.97 (s, 3H) | LCMSA038: 521.20 [M + H]⁺; Rt: 1.66 min |
| 219 | 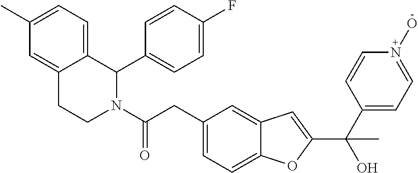 4-(1-(5-(2-(1-(4-fluorophenyl)-6- methyl-3,4-dihydroisoquinolin- 2(1H)-yl)-2- oxoethyl)benzofuran-2-yl)-1- hydroxyethyl)pyridine 1-oxide | ¹H NMR (MeOD, 400 MHz): δ 8.30 (d, J = 4.0 Hz, 2H), 7.69 (d, J = 8.0 Hz, 2H), 7.44-7.35 (m, 2H), 7.20-7.17 (m, 3H), 7.01-6.92 (m, 5H), 6.80-6.76 (m, 2H), 3.95-3.94 (m, 3H), 3.33-3.32 (m, 1H), 2.66- 2.64 (m, 2H), 2.31 (s, 3H), 1.95 (s, 3H). | LC-MS (020): 537.3 [M + H]⁺; Rt: 1.78 min |
| 220 | 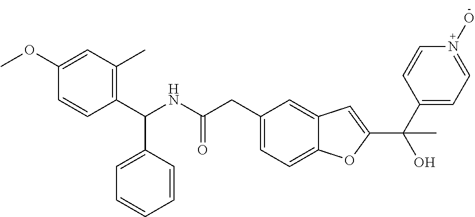 4-(1-hydroxy-1-(5-(2-(((4- methoxy-2- methylphenyl)(phenyl)methyl) amino)-2-oxoethyl)benzofuran-2- yl)ethyl)pyridine 1-oxide | ¹H NMR (MeOD, 400 MHz): δ 8.27 (d, J = 4.0 Hz, 2H), 7.66 (d, J = 4.0 Hz, 2H), 7.51 (s, 1H), 7.36-7.24 (m, 7H), 6.95 (d, J = 8.0 Hz, 1H), 6.77-6.64 (m, 3H), 6.27 (s, 1H), 3.74 (s, 3H), 3.65 (s, 2H), 2.18 (s, 3H), 1.95 (s, 3H). | LC-MS (020): 523.2 [M + H]⁺; Rt: 1.65 min |
| 221 | 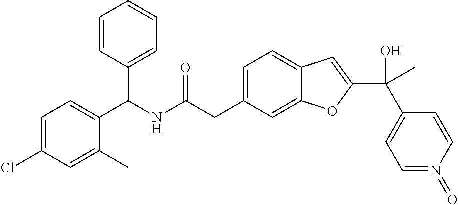 [SMILES: Cc1cc(Cl)ccc1C(NC(=O)Cc1ccc2cc(C(C)(O)c3cc[n+]([O-])cc3)oc2c1)c1ccccc1] 4-(1-(6-(2-(((4-chloro-2- methylphenyl)(phenyl)methyl) amino)-2-oxoethyl)benzofuran-2- yl)-1-hydroxyethyl)pyridine 1- oxide | ¹H NMR (MeOD, 400 MHz): δ 8.31 (d, J = 7.6 Hz, 2H), 7.70 (d, J = 7.2 Hz, 2H), 7.53 (s, 1H), 7.39-7.20 (m, 8H), 7.09 (s, 1H), 7.00 (d, J = 8.0 Hz, 1H), 6.79 (s, 1H), 6.12 (s, 1H), 3.68 (d, J = 2.0 Hz, 2H), 2.26 (s, 3H), 1.97 (s, 3H) | LCMSA020: 527 [M + H]⁺; Rt: 1.74 min |
| 222 | 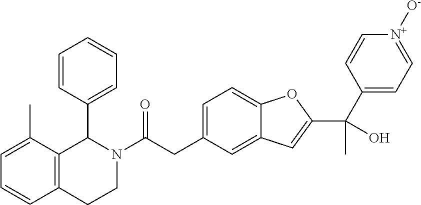 [SMILES: Cc1cccc2c1C(c1ccccc1)N(C(=O)Cc1ccc3oc(C(C)(O)c4cc[n+]([O-])cc4)cc3c1)CC2] 4-(1-hydroxy-1-(5-(2-(8-methyl- 1-phenyl-3,4- dihydroisoquinolin-2(1H)-yl)-2- oxoethyl)benzofuran-2- yl)ethyl)pyridine 1-oxide | ¹H NMR (MeOD, 400 MHz): δ 8.19-8.17 (m, 2H), 7.57-7.25 (m, 6H), 7.08-6.88 (m, 7H), 6.63 (s, 1H), 3.84-3.83 (m, 3H), 3.20-3.22 (m, 1H), 2.57-2.56 (m, 2H), 1.91 (s, 3H), 1.83 (s,3H). | LC-MS (020): 519.2 [M+H]⁺; Rt : 1.59 min |
| 223 | 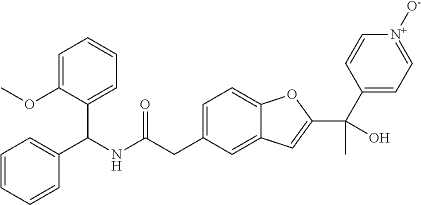 [SMILES: COc1ccccc1C(NC(=O)Cc1ccc2oc(C(C)(O)c3cc[n+]([O-])cc3)cc2c1)c1ccccc1] 4-(1-hydroxy-1-(5-(2-(((2- methoxyphenyl)(phenyl)methyl) amino)-2-oxoethyl)benzofuran- 2-yl)ethyl)pyridine 1-oxide | ¹H NMR (400 MHz, MEOD) δ: 8.32-8.30 (m, 2H), 7.70-7.69 (m, 2H), 7.54 (s, 1H), 7.38 (s, 1H), 7.27-7.14 (m, 8H), 6.93-6.90 (m, 2H), 6.81 (s, 1H), 6.40 (s, 1H), 3.70 (s, 2H), 3.60 (s, 3H), 1.97 (s, 3H). | LC-MS (020): 509.2 [M + H]⁺: Rt: 1.47 min |
| 224 | 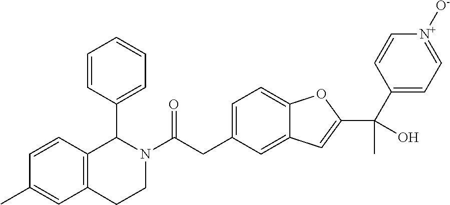 [SMILES: Cc1ccc2c(c1)CCN(C(=O)Cc1ccc3oc(C(C)(O)c4cc[n+]([O-])cc4)cc3c1)C2c1ccccc1] 4-(1-hydroxy-1-(5-(2-(6-methyl- 1-phenyl-3,4- dihydroisoquinolin-2(1H)-yl)-2- oxoethyl)benzofuran-2- yl)ethyl)pyridine 1-oxide | ¹H NMR (MeOD, 400 MHz): δ 8.17 (d, J = 8.0 Hz, 2H), 7.56 (d, J = 4.0 Hz, 2H), 7.32-7.22 (m, 2H), 7.13- 7.03 (m, 6H), 6.90-6.80 (m, 3H), 6.69-6.62 (m, 2H), 3.83-3.78 (m, 3H), 3.28-3.27 (m, 1H), 2.52-2.49 (m, 2H), 2.18 (s, 3H), 1.83 (s, 3H). | LC-MS (020): 519.2 [M + H]⁺; Rt: 1.60 min |
| 225 | 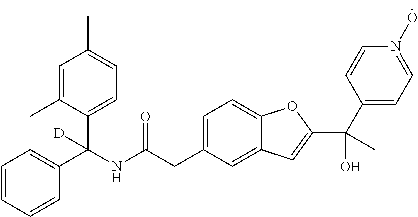 4-(1-(5-(2-((deutero-(2,4- dimethylphenyl)(phenyl)methyl) amino)-2-oxoethyl)benzofuran- 2-yl)-1-hydroxyethyl)pyridine 1- oxide | ¹H NMR (MeOD, 400 MHz): δ 8.30 (d, J = 7.2 Hz, 2H), 7.70 (d, J = 6.8 Hz, 2H), 7.52 (s, 1H), 7.38-7.23 (m, 5H), 7.15 (d, J = 7.2 Hz, 2H), 6.99-6.93 (m, 3H), 6.79 (s, 1H), 3.67 (s, 2H), 2.28 (s, 3H), 2.18 (s, 3H), 1.97 (s, 3H) | LCMSA020: 508 [M + H]⁺; Rt: 1.55 min |
| 226 | 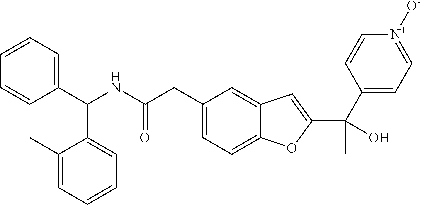 [SMILES: Cc1ccccc1C(NC(=O)Cc1ccc2oc(C(C)(O)c3cc[n+]([O-])cc3)cc2c1)c1ccccc1] 4-(1-hydroxy-1-(5-(2-oxo-2- ((phenyl(o- tolyl)methyl)amino)ethyl) benzofuran-2-yl)ethyl)pyridine 1-oxide | ¹H NMR (400 MHz, MEOD) δ: 8.31 (d, J = 4.0 Hz, 2H), 7.69 (d, J = 8.0 Hz, 2H), 7.53 (s, 1H), 7.35-7.25 (m, 5H), 7.17-7.11 (m, 6H), 6.79 (s, 1H), 6.34 (s, 1H), 3.67 (s, 2H), 2.21 (s, 3H), 1.96 (s, 3H) | LC-MS (020): 493.2 [M + H]⁺; Rt: 1.51 min |
| 227 | 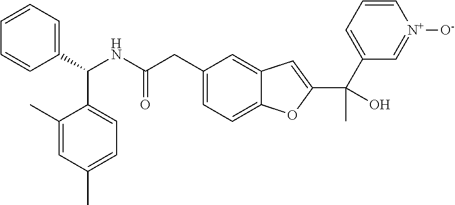 [SMILES: Cc1ccc([C@@H](NC(=O)Cc2ccc3oc(C(C)(O)c4ccc[n+]([O-])c4)cc3c2)c2ccccc2)c(C)c1] 3-(1-(5-(2-(((S)-(2,4- dimethylphenyl)(phenyl)methyl) amino)-2-oxoethyl)benzofuran- 2-yl)-1-hydroxyethyl)pyridine 1- oxide | ¹H NMR (DMSO, 400 MHz): δ 8.807 (d, J = 8.4 Hz, 1H), 8.43 (s, 1H), 8.17 (d, J = 8.0 Hz, 1H), 7.56 (d, J = 8.0 Hz, 1H), 7.43 (t, J = 6.4 Hz, 1H); 7.31-7.14 (m, 7H), 7.08 (d, J = 10 Hz, 1H), 6.97 (d, J = 5.6 Hz, 3H), 6.80 (d, J = 8.0 Hz, 1H), 6.52 (s, 1H), 6.16 (d, J = 7.6 Hz, 1H), 3.35 (s, 2H), 2.23 (s, 3H), 2.14 (s, 3H), 1.73 (s, 3H). | LCMSA020: 507.7 [M + H]⁺; Rt: 1.53 min |
| 228 | 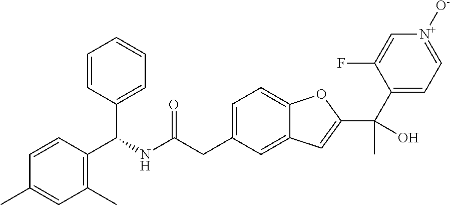 [SMILES: Cc1ccc([C@@H](NC(=O)Cc2ccc3oc(C(C)(O)c4cc[n+]([O-])cc4F)cc3c2)c2ccccc2)c(C)c1] 4-(1-(5-(2-(((S)-(2,4- dimethylphenyl)(phenyl)methyl) amino)-2-oxoethyl)benzofuran- 2-yl)-1-hydroxyethyl)-3- fluoropyridine 1-oxide | ¹H NMR (MeOD, 400 MHz): δ 8.37 (d, J = 5.2 Hz, 1H), 8.28 (d, J = 6.4 Hz, 1H), 7.93 (dd, J₁ = 6.8 Hz, J₂ = 8.4 Hz, 1H), 7.53 (s, 1H), 7.37- 7.24 (m, 5H), 7.15 (d, J = 6.8 Hz, 2H), 6.99-6.92 (m, 3H), 6.78 (s, 1H), 6.30 (s, 1H), 3.67 (s, 2H), 2.28 (s, 3H), 2.17 (s, 3H), 2.03 (s, 3H) | LCMSA038: 525 [M + H]⁺; Rt: 1.68 min |
| 229 | 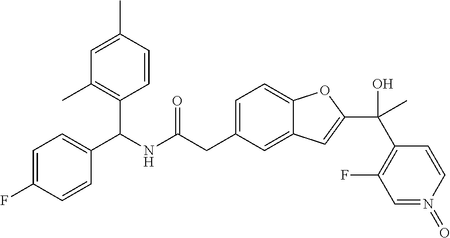 [SMILES: Cc1ccc(C(NC(=O)Cc2ccc3oc(C(C)(O)c4cc[n+]([O-])cc4F)cc3c2)c2ccc(F)cc2)c(C)c1] 4-(1-(5-(2-(((2,4- dimethylphenyl)(4- fluorophenyl)methyl)amino)-2- oxoethyl)benzofuran-2-yl)-1- hydroxyethyl)-3-fluoropyridine 1-oxide | ¹H NMR (MEOD, 400 MHz): δ 8.36-8.34 (m, 1H), 8.28-8.26 (m, 1H), 7.93-7.89 (m, 1H), 7.52 (s, 1H), 7.35 (d, J = 4.0 Hz, 1H), 7.24- 7.22 (m, 1H), 7.16-7.16 (m, 2H), 7.03-6.94 (m, 5H), 6.77 (s, 1H), 6.28 (s, 1H), 3.65 (s, 2H), 2.27 (m, 3H), 2.16 (s, 3H), 2.02 (s, 3H). | LC-MS(020): 543.2 [M + H]⁺; Rt: 1.74 min |
| 230 | 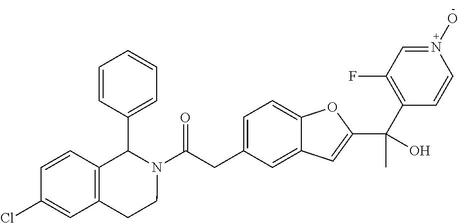 4-(1-(5-(2-(6-chloro-1-phenyl- 3,4-dihydroisoquinolin-2(1H)- yl)-2-oxoethyl)benzofuran-2-yl)- 1-hydroxyethyl)-3- fluoropyridine 1-oxide | ¹H NMR (MEOD, 400 MHz): δ 8.01 (d, J = 8.0 Hz, 1H), 7.94 (s, 1H), 7.66 (s, 1H), 7.31-7.26 (m, 3H), 7.16-7.06 (m, 6H), 6.99 (d, J = 4.0 Hz, 1H), 6.90 (s, 1H), 6.54 (d, J = 4.0 Hz, 1H), 3.86-3.79 (m, 3H), 3.42-3.34 (m, 1H), 2.68 (s, 2H), 2.01 (s, 3H). | LC-MS(020): 557.2 [M + H]⁺; Rt: 1.80 min |
| 231 | 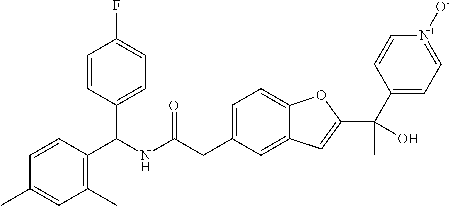 [SMILES: Cc1ccc(C(NC(=O)Cc2ccc3oc(C(C)(O)c4cc[n+]([O-])cc4)cc3c2)c2ccc(F)cc2)c(C)c1] 4-(1-(5-(2-(((2,4- dimethylphenyl)(4- fluorophenyl)methyl)amino)-2- oxoethyl)benzofuran-2-yl)-1- hydroxyethyl)pyridine 1-oxide | ¹H NMR (MeOD, 400 MHz): δ 8.31 (d, J = 6.4 Hz, 2H), 7.69 (d, J = 6.4 Hz, 2H), 7.52 (s, 1H), 7.37 (d, J = 8.4 Hz, 1H), 7.24 (d, J = 1.2 Hz, 1H), 7.15 (m, J = 8.0 Hz, 2H), 7.04- 6.96 (m, 5H), 6.79 (s, 1H), 6.29 (s, 1H), 3.66 (s, 2H), 2.28 (s, 3H), 2.17 (s, 3H), 1.97 (s, 3H). | LCMSA (044): 525.20 [M + H]⁺; Rt: 1.74 min |
| 232 | 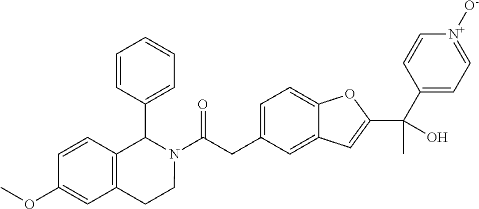 [SMILES: COc1ccc2c(c1)CCN(C(=O)Cc1ccc3oc(C(C)(O)c4cc[n+]([O-])cc4)cc3c1)C2c1ccccc1] 4-(1-hydroxy-1-(5-(2-(6- methoxy-1-phenyl-3,4- dihydroisoquinolin-2(1H)-yl)-2- oxoethyl)benzofuran-2- yl)ethyl)pyridine 1-oxide | ¹H NMR (MeOD, 400 MHz): δ 8.31 (d, J = 12 Hz, 2H), 7.69 (d, J = 6.8 Hz, 2H), 7.45-7.16 (m, 8H), 6.98 (d, J = 8.4 Hz, 1H), 6.81-6.71 (m, 4H), 3.96-3.91 (m, 2H), 3.78 (s, 3H), 3.43-3.36 (m, 2H), 2.66 (s, 2H), 1.96 (s, 3H). | LCMSA(036): 535.20 [M + H]⁺; Rt: 1.55 min |
| 233 | 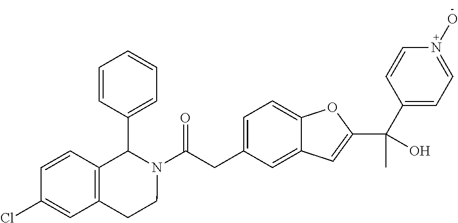 4-(1-(5-(2-(6-chloro-1-phenyl- 3,4-dihydroisoquinolin-2(1H)- yl)-2-oxoethyl)benzofuran-2-yl)- 1-hydroxyethyl)pyridine 1-oxide | ¹H NMR (CDCL3, 400 MHz): δ 8.04 (d, J = 4.0 Hz, 2H), 7.35 (d, J = 8.0 Hz, 3H), 7.30-7.26 (m, 2H), 7.19-7.08 (m, 6H), 7.00 (d, J = 8.0 Hz, 1H), 6.93 (s, 1H), 6.58 (d, J = 4.0 Hz, 1H), 3.94-3.80 (m, 3H), 3.42-3.35 (m, 1H), 2.68 (d, J = 4.0 Hz, 2H), 1.93 (s, 3H). | LC-MS (020): 539.2 [M + H]⁺; Rt: 1.76 min |
| 234 | 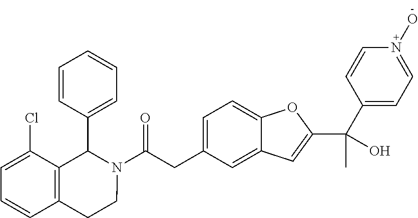 4-(1-(5-(2-(8-chloro-1-phenyl- 3,4-dihydroisoquinolin-2(1H)- yl)-2-oxoethyl)benzofuran-2-yl)- 1-hydroxyethyl)pyridine 1-oxide | ¹H NMR (CDCL3, 400 MHz): δ 8.14 (d, J = 4.0 Hz, 2H), 7.45-7.39 (m, 3H), 7.34-6.99 (m, 11H), 6.62- 6.61 (m, 1H), 3.87-3.78 (m, 3H), 3.32-3.28 (m, 1H), 2.76-2.74 (m, 2H), 2.01 (s, 3H). | LC-MS (020): 539.2 [M + H]⁺; Rt: 1.71 min |
| 235 | 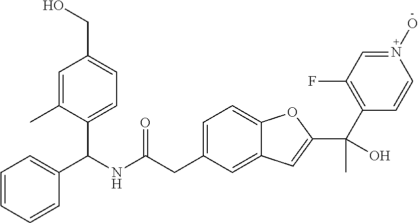 3-fluoro-4-(1-hydroxy-1-(5-(2- (((4-(hydroxymethyl)-2- methylphenyl)(phenyl)methyl) amino)-2-oxoethyl)benzofuran-2- yl)ethyl)pyridine 1-oxide | ¹H NMR (MeOD, 400 MHz): δ 8.39-8.37 (m, 1H), 8.30-8.28 (m, 1H), 7.95-7.91 (m, 1H), 7.53 (s, 1H), 7.37-7.08 (m, 10H), 6.79 (s, 1H), 6.33 (s, 1H), 4.56 (s, 2H), 3.68 (s, 2H), 2.22 (s, 3H), 2.05 (s, 3H) | LCMSA044: 541 [M + H]⁺; Rt: 1.55 min |
| 236 | 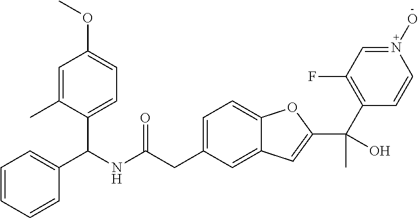 3-fluoro-4-(1-hydroxy-1-(5-(2- (((4-methoxy-2- methylphenyl)(phenyl)methyl) amino)-2-oxoethyl)benzofuran-2- yl)ethyl)pyridine 1-oxide | ¹H NMR (MeOD, 400 MHz): δ 8.38-8.36 (m, 1H), 8.28 (d, J = 5.2 Hz, 1H), 7.94-7.91 (m, 1H), 7.53(s, 1H), 7.37-7.15 (m, 7H), 6.95 (d, J = 6.8 Hz, 1H), 6.78-6.66 (m, 3H), 6.28 (s, 1H), 3.77 (s, 3H), 3.67 (s, 2H), 2.20 (s, 3H), 2.03 (s, 3H) | LCMSA044: 541 [M + H]⁺; Rt: 1.68 min |
| 237 | 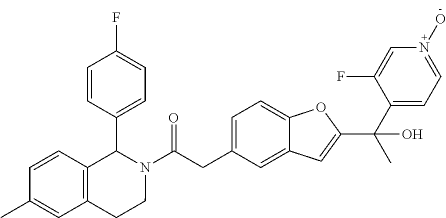 3-fluoro-4-(1-(5-(2-(1-(4- fluorophenyl)-6-methyl-3,4- dihydroisoquinolin-2(1H)-yl)-2- oxoethyl)benzofuran-2-yl)-1- hydroxyethyl)pyridine 1-oxide | ¹H NMR (MeOD, 400 MHz): δ 8.25 (d, J = 6.0 Hz, 1H), 8.17 (d, J = 6.8 Hz, 1H), 7.85 (m, 1H), 7.32 (s, 1H), 7.24 (d, J = 8.4 Hz, 1H), 7.05 (m, 3H), 6.92-6.81 (m, 5H), 6.68 (s, 1H), 6.63 (s, 1H), 3.84 (m, 2H), 2.54 (m, 2H), 2.20 (s, 3H), 1.91 (s, 3H), 1.19 (s, 2H) | LCMS: 555.2 [M + H]⁺; Rt: 1.69 min |
| 238 | 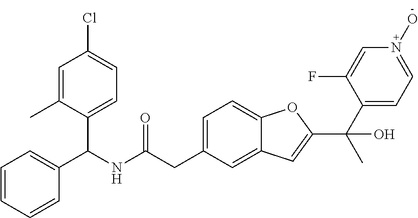 4-(1-(5-(2-(((4-chloro-2- methylphenyl)(phenyl)methyl) amino)-2-oxoethyl)benzofuran-2- yl)-1-hydroxyethyl)-3- fluoropyridine 1-oxide | ¹H NMR (MeOD, 400 MHz): δ 8.38 (d, J = 1.6 Hz, 1H), 8.37-8.27 (m, 1H), 7.93 (d, J = 9.2 Hz, 1H), 7.53 (s, 1H), 7.38-7.20 (m, 8H), 7.00 (s, 1H), 6.98 (d, J = 6.0 Hz, 1H), 6.78 (s, 1H), 6.12 (s, 1H), 3.68 (d, J = 2.8 Hz, 2H), 2.26 (s, 3H), 2.03 (s, 3H) | LCMS: 545.1 [M + H]⁺; Rt: 1.65 min |
| 239 | 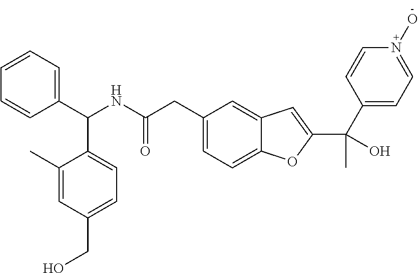 4-(1-hydroxy-1-(5-(2-(((4- (hydroxymethyl)-2- methylphenyl)(phenyl)methyl) amino)-2-oxoethyl)benzofuran-2- yl)ethyl)pyridine 1-oxide | ¹H NMR (MeOD, 400 MHz): δ 8.31 (d, J = 6.8 Hz, 2H), 7.70 (d, J = 6.8 Hz, 2H), 7.53 (s, 1H), 7.36-7.09 (m, 10H), 6.80 (s, 1H), 6.33 (s, 1H), 4.56 (s, 2H), 3.68 (s, 2H), 2.22 (s, 3H), 1.97 (s, 3H) | LCMSA044: 523 [M + H]⁺; Rt: 1.52 min |
| 240 | 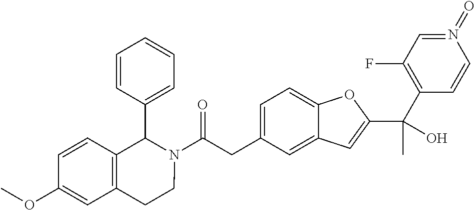 [SMILES: COc1ccc2c(c1)CCN(C(=O)Cc1ccc3oc(C(C)(O)c4cc[n+]([O-])cc4F)cc3c1)C2c1ccccc1] 3-fluoro-4-(1-hydroxy-1-(5-(2- (6-methoxy-1-phenyl-3,4- dihydroisoquinolin-2(1H)-yl)-2- oxoethyl)benzofuran-2- yl)ethyl)pyridine 1-oxide | ¹H NMR (MeOD, 400 MHz): δ 8.35 (d, J = 6.0 Hz, 1H), 8.26 (d, J = 6.8 Hz, 1H), 7.93-7.89 (m, 1H), 7.44- 7.11 (m, 8H), 6.78 (d, J = 8.0 Hz, 1H), 6.80-6.71 (m, 4H), 4.11-3.90 (m, 3H), 3.78 (s, 3H), 3.43-3.36 (m, 1H), 2.69-2.62 (m, 2H), 2.01 (s, 3H). | LCMS (036): 553 [M + H]⁺; Rt: 1.59 min |

Following essentially the procedure as described in Example 94, the compound in Table 22 was prepared.

TABLE 22

| Example | Structure/Name | NMR | LCMS |
|---|---|---|---|
| 241 | 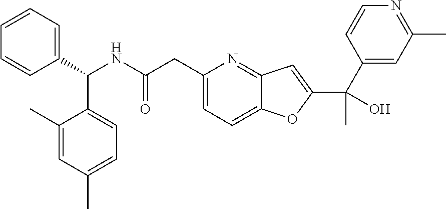 [SMILES: Cc1ccc([C@@H](NC(=O)Cc2ccc3oc(C(C)(O)c4ccnc(C)c4)cc3n2)c2ccccc2)c(C)c1] N-((S)-(2,4- dimethylphenyl)(phenyl) methyl)-2-(2-(1-hydroxy-1-(2- methylpyridin-4- yl)ethyl)furo[3,2-b]pyridin-5- yl)acetamide | ¹H NMR (MeOD, 400 MHz): δ 8.66 (d, J = 6.4 Hz, 1H), 8.14 (s, 1H), 8.07 (d, J = 6.8 Hz, 1H), 7.89 (d, J = 8.4 Hz, 1H), 7.38-7.20 (m, 6H), 7.07- 6.97 (m, 4H), 6.33 (s, 1H), 3.94 (s, 2H), 2.80 (s, 3H), 2.29 (s, 3H), 2.21 (s, 3H), 2.07 (s, 3H). | LCMSA036: 506 [M + H]⁺; Rt: 1.28 min |
| 242 | 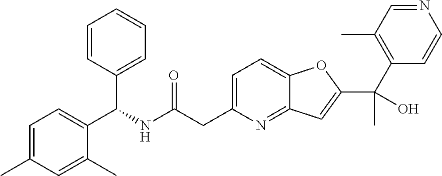 [SMILES: Cc1ccc([C@@H](NC(=O)Cc2ccc3oc(C(C)(O)c4ccncc4C)cc3n2)c2ccccc2)c(C)c1] N-((S)-(2,4- dimethylphenyl)(phenyl) methyl)-2-(2-(1-hydroxy-1-(3- methylpyridin-4- yl)ethyl)furo[3,2-b]pyridin-5- yl)acetamide | ¹H NMR (CDCl₃, 400 MHz): δ 8.42 (d, J = 5.2 Hz, 1H), 8.28 (s, 1H), 7.79 (d, J = 8.4 Hz, 1H), 7.73 (d, J = 5.2 Hz, 1H), 7.30-7.19 (m, 7H), 7.05-6.95 (m, 5H), 6.33 (s, 1H), 3.91 (s, 2H), 2.28 (s, 3H), 2.21 (s, 3H), 2.15 (s, 3H), 2.02 (s, 3H). | LC-MS044: 506.2 [M + H]⁺; Rt = 1.65 min |
| 243 | 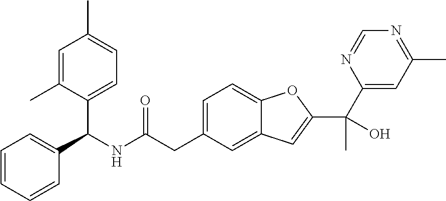 [SMILES: Cc1ccc([C@@H](NC(=O)Cc2ccc3oc(C(C)(O)c4cc(C)ncn4)cc3c2)c2ccccc2)c(C)c1] N-((S)-(2,4- dimethylphenyl)(phenyl) methyl)-2-(2-(1-hydroxy-1-(6- methylpyrimidin-4- yl)ethyl)benzofuran-5- yl)acetamide | ¹H NMR (CDCl₃, 400 MHz): δ 8.94 (s, 1H), 8.79 (s, 1H), 7.79 (s, 1H), 7.51 (s, 1H), 7.34-7.13 (m, 8H), 6.99-6.94 (m, 3H), 6.74 (s, 1H), 6.30-6.28 (m, 1H), 3.66 (s, 2H), 2.59 (s, 3H), 2.28 (s, 3H), 2.17 (s, 3H), 1.98 (s, 3H). | LC-MS036: 506.2 [M + H]⁺; Rt = 1.73 min |
| 244 | 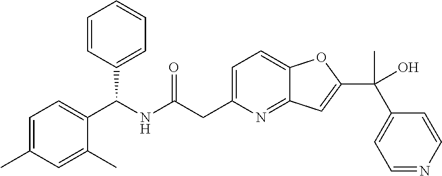 [SMILES: Cc1ccc([C@@H](NC(=O)Cc2ccc3oc(C(C)(O)c4ccncc4)cc3n2)c2ccccc2)c(C)c1] N-((S)-(2,4- dimethylphenyl)(phenyl) methyl)-2-(2-(1-hydroxy-1- (pyridin-4-yl)ethyl)furo[3,2- b]pyridin-5-yl)acetamide | ¹H NMR (MeOD, 400 MHz): δ 8.54 (d, J = 6.0 Hz, 2H), 7.82 (d, J = 8.4 Hz, 1H), 7.62 (d, J = 6.4 Hz, 2H), 7.31-7.25 (m, 4H), 7.21 (d, J = 7.2 Hz, 2H), 7.05-6.96 (m, 3H), 6.92 (s, 1H), 6.33 (s, 1H), 3.90 (s, 2H), 2.30 (s, 3H), 2.21 (s, 3H), 1.99 (s, 3H). | LCMSA(044): 492.20 [M + H]⁺; Rt: 1.62 min |
| 245 | 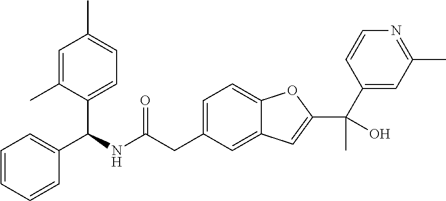 [SMILES: Cc1ccc([C@@H](NC(=O)Cc2ccc3oc(C(C)(O)c4ccnc(C)c4)cc3c2)c2ccccc2)c(C)c1] N-((S)-(2,4- dimethylphenyl)(phenyl) methyl)-2-(2-(1-hydroxy-1-(2- methylpyridin-4- yl)ethyl)benzofuran-5- yl)acetamide | ¹H NMR (MeOD, 400 MHz): δ 8.63 (d, J = 6.4 Hz, 1H), 8.09 (s, 1H), 8.01 (d, J = 6.0 Hz, 1H), 7.55 (s, 1H), 7.36-7.25 (m, 5H), 7.16 (s, 1H), 7.14 (s, 1H), 7.00-6.90 (m, 4H), 6.30 (s, 1H), 3.67 (s, 2H), 2.78 (s, 3H), 2.28 (s, 3H), 2.18 (s, 3H), 2.03 (s, 3H) | LCMSA036: 505.2 [M + H]⁺; Rt: 1.41 min |

Following essentially the procedure as described in Example 47, the compound in Table 23 was prepared.

TABLE 23

| Example | Structure/Name | NMR | LCMS |
|---|---|---|---|
| 246 | 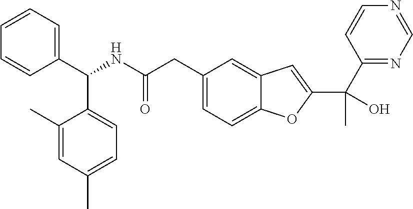 [SMILES: Cc1ccc([C@@H](NC(=O)Cc2ccc3oc(C(C)(O)c4ccncn4)cc3c2)c2ccccc2)c(C)c1] N-((S)-(2,4- dimethylphenyl)(phenyl)methyl)- 2-(2-(1-hydroxy-1-(pyrimidin- 4-yl)ethyl)benzofuran-5- yl)acetamide | ¹H NMR (MeOD, 400 MHz): δ 9.10 (s, 1H), 8.83 (d, J = 4.8 Hz, 1H), 7.91 (d, J = 5.2 Hz, 1H), 7.51 (s, 1H), 7.34-7.21 (m, 5H), 7.14 (d, J = 7.2 Hz, 2H), 6.99-6.92 (m, 3H), 6.76 (s, 1H), 6.29 (s, 1H), 3.66 (s, 2H), 2.28 (s, 3H), 2.17 (s, 3H), 2.00 (d, J = 7.2 Hz, 3H). | LCMSA038: 492.4 [M + H]⁺; Rt = 1.727 min |
| 247 | 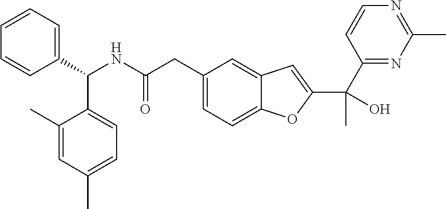 [SMILES: Cc1ccc([C@@H](NC(=O)Cc2ccc3oc(C(C)(O)c4ccnc(C)n4)cc3c2)c2ccccc2)c(C)c1] N-((S)-(2,4- dimethylphenyl)(phenyl)methyl)- 2-(2-(1-hydroxy-1-(2- methylpyrimidin-4- yl)ethyl)benzofuran-5- yl)acetamide | ¹H NMR (MeOD, 400 MHz): δ 8.71 (d, J = 5.6 Hz, 1H), 7.71 (d, J = 5.2 Hz, 1H), 7.51 (s, 1H), 7.34-7.22 (m, 5H), 7.14 (d, J = 6.8 Hz, 2H), 6.99-6.92 (m, 3H), 6.75 (s, 1H), 6.29 (s, 1H), 3.66 (s, 2H), 2.68 (s, 3H), 2.28 (s, 3H), 2.17 (s, 3H), 1.99 (s, 3H). | LCMSA036: 506.2 [M + H]⁺; Rt = 1.739 min |

Following essentially the procedure as described in Example 105, the compound in Table 24 was prepared.

TABLE 24

| Example | Structure/Name | NMR | LCMS |
|---|---|---|---|
| 248 | 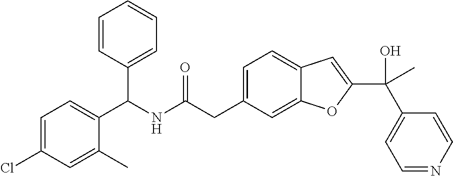 [SMILES: Cc1cc(Cl)ccc1C(NC(=O)Cc1ccc2cc(C(C)(O)c3ccncc3)oc2c1)c1ccccc1] N-((4-chloro-2- methylphenyl)(phenyl)methyl)- 2-(2-(1-hydroxy-1-(pyridin-4- yl)ethyl)benzofuran-6- yl)acetamide | ¹H NMR (MeOD, 400 MHz): δ 8.51 (d, J = 6.0 Hz, 2H), 7.59-7.57 (m, 2H), 7.53 (s, 1H), 7.38-7.20 (m, 8H), 7.08 (s, 1H), 6.99 (d, J = 8.4 Hz, 1H), 6.76 (s, 1H), 6.12 (s, 1H), 3.68 (d, J = 2.8 Hz, 2H), 2.25 (d, J = 2.8 Hz, 3H), 1.96 (s, 3H) | LCMSA020: 511 [M + H]⁺; Rt: 1.85 min |
| 249 | 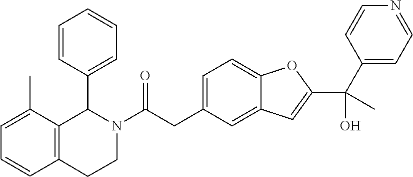 [SMILES: Cc1cccc2c1C(c1ccccc1)N(C(=O)Cc1ccc3oc(C(C)(O)c4ccncc4)cc3c1)CC2] 2-(2-(1-hydroxy-1-(pyridin-4- yl)ethyl)benzofuran-5-yl)-1-(8- methyl-1-phenyl-3,4- dihydroisoquinolin-2(1H)- yl)ethanone | ¹H NMR (MeOD, 400 MHz): δ 8.37- 8.36 (m, 2H), 7.43-7.41 (m, 3H), 7.32-7.19 (m, 4H), 7.13-6.85 (m, 7H), 6.58 (s, 1H), 3.82-3.75 (m, 3H), 3.21-3.15 (m, 1H), 2.55-2.53 (m, 2H), 1.88 (s, 3H), 1.79 (s, 3H). | LC-MS (020): 503.2 [M + H]⁺; Rt: 1.72 min |
| 250 | 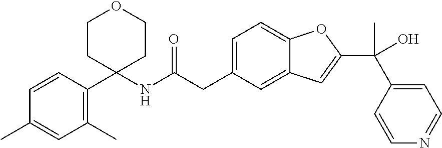 [SMILES: Cc1ccc(C2(NC(=O)Cc3ccc4oc(C(C)(O)c5ccncc5)cc4c3)CCOCC2)c(C)c1] N-(4-(2,4- dimethylphenyl)tetrahydro-2H- pyran-4-yl)-2-(2-(1-hydroxy-1- (pyridin-4-yl)ethyl)benzofuran- 5-yl)acetamide | 1H NMR (MeOD, 400 MHz): δ 8.40 (d, J = 6.0 Hz, 2H), 7.46 (d, J = 6 Hz, 2H), 7.36 (s, 1H), 7.24 (d, J = 8.4 Hz, 1H), 7.15 (d, J = 8.0 Hz, 1H), 7.09 (d, J = 8.4 Hz, 1H), 6.83 (d, J = 8.4 Hz, 1H), 6.77 (s, 1H), 6.63 (s, 1H), 3.68-3.58 (m, 4H), 3.50 (s, 2H), 2.40-2.36 (m, 2H), 2.26 (s, 3H), 2.12 (s, 3H), 1.98-1.91 (m, 2H), 1.84 (s, 3H). | LCMSA044: 485.70 [M + H]⁺; Rt = 1.678 min |
| 251 | 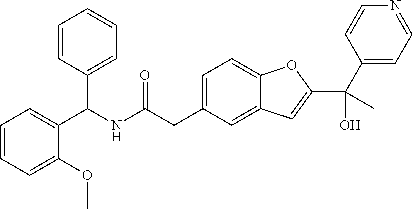 [SMILES: COc1ccccc1C(NC(=O)Cc1ccc2oc(C(C)(O)c3ccncc3)cc2c1)c1ccccc1] 2-(2-(1-hydroxy-1-(pyridin-4- yl)ethyl)benzofuran-5-yl)-N-((2- methoxyphenyl)(phenyl)methyl) acetamide | ¹H NMR (400 MHz, MEOD) δ: 8.52-8.50 (m, 2H), 7.58-7.53 (m, 3H), 7.38 (d, J = 8.0 Hz, 1H), 7.27-6.92 (m, 8H), 6.90-6.77 (m, 2H), 6.40 (s, 1H), 5.50 (s, 1H), 3.69 (s, 2H), 3.58 (s, 3H), 1.96 (s, 3H). | LC-MS (044): 493.2 [M + H]⁺; Rt: 1.70 min |
| 252 | 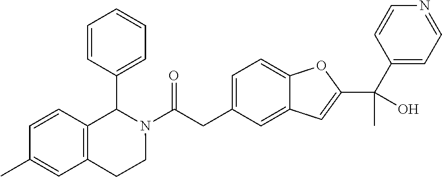 [SMILES: Cc1ccc2c(c1)CCN(C(=O)Cc1ccc3oc(C(C)(O)c4ccncc4)cc3c1)C2c1ccccc1] 2-(2-(1-hydroxy-1-(pyridin-4- yl)ethyl)benzofuran-5-yl)-1-(6- methyl-1-phenyl-3,4- dihydroisoquinolin-2(1H)- yl)ethanone | ¹H NMR (MeOD, 400 MHz): δ 8.39 (d, J = 4.0 Hz, 2H), 7.45-7.44 (m, 2H), 7.31-7.22 (m, 2H), 7.15-7.03 (m, 6H), 6.88-6.70 (m, 3H), 6.70 (s, 1H), 6.59 (s, 1H), 3.83-3.82 (m, 3H), 3.32-3.27 (m, 1H), 2.52 (s, 2H), 2.19 (s, 3H), 1.82 (s, 3H). | LC-MS (020): 503.2 [M + H]⁺; Rt: 1.73 min |
| 253 | 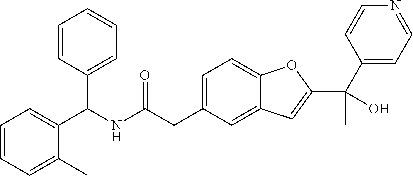 [SMILES: Cc1ccccc1C(NC(=O)Cc1ccc2oc(C(C)(O)c3ccncc3)cc2c1)c1ccccc1] 2-(2-(1-hydroxy-1-(pyridin-4- yl)ethyl)benzofuran-5-yl)-N- (phenyl(o- tolyl)methyl)acetamide | ¹H NMR (400 MHz, dmso) δ: 8.91 (d, J = 8.0 Hz, 1H), 8.51 (d, J = 4.0 Hz, 2H), 7.47-7.38 (m, 4H), 7.31- 7.24 (m, 3H), 7.18-7.11 (m, 7H), 6.79 (s, 1H), 6.34 (s, 1H), 6.22 (d, J = 8.0 Hz, 1H), 3.58 (s, 2H), 2.18 (s, 3H), 1.84 (s, 3H). | LC-MS (020): 477.2 [M + H]⁺; Rt: 1.61 min |
| 254 | 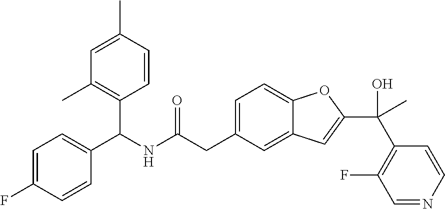 [SMILES: Cc1ccc(C(NC(=O)Cc2ccc3oc(C(C)(O)c4ccncc4F)cc3c2)c2ccc(F)cc2)c(C)c1] N-((2,4-dimethylphenyl)(4- fluorophenyl)methyl)-2-(2-(1-(3- fluoropyridin-4-yl)-1- hydroxyethyl)benzofuran-5- yl)acetamide | ¹H NMR (CDCl₃, 400 MHz): δ 8.48 (d, J = 4.8 Hz, 1H), 8.38 (d, J = 2.8 Hz, 1H), 7.89 (m, 1H), 7.52 (s, 1H), 7.35 (d, J = 8.4 Hz, 1H), 7.24-6.95 (m, 8H), 6.73 (s, 1H), 6.29 (d, J = 4.8 Hz, 1H), 3.67 (s, 2H), 2.28 (s, 3H), 2.17 (s, 3H), 2.04 (s, 3H). | LC-MS (020): 527.2 [M + H]⁺; Rt = 1.86 min |

Following essentially the procedure as described in Example 45, the compound in Table 25 was prepared.

TABLE 25

| Example | Structure/Name | NMR | LCMS |
|---|---|---|---|
| 255 | 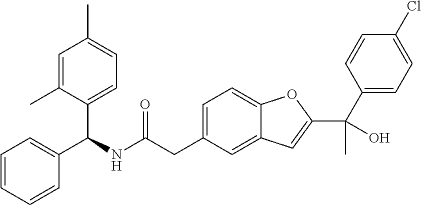 [SMILES: Cc1ccc([C@@H](NC(=O)Cc2ccc3oc(C(C)(O)c4ccc(Cl)cc4)cc3c2)c2ccccc2)c(C)c1] 2-(2-(1-(4-chlorophenyl)-1- hydroxyethyl)benzofuran-5-yl)- N-((S)-(2,4- dimethylphenyl)(phenyl)methyl) acetamide | ¹H-NMR (MeOD, 400 MHz): δ 7.52 (d, J = 4.8 Hz, 2H), 7.48 (s, 1H), 7.37 (d, J = 5.2 Hz, 2H), 7.33 (d, J = 2.8 Hz, 1H), 7.27-7.17 (m, 4H), 7.14 (d, J = 4.4 Hz, 2H), 6.99 (s, 1H), 6.94 (d, J = 0.8 Hz, 2H), 6.66 (s, 1H), 6.30 (s, 1H), 3.67 (s, 2H), 2.28 (s, 3H), 2.17 (s, 3H), 1.94 (s, 3H). | LC-MS (P1): 524.2 [M + H]⁺; Rt: 1.62 min |

Following essentially the procedure as described in Example 28, the compound in Table 26 was prepared.

TABLE 25

| Example | Structure/Name | NMR | LCMS |
|---|---|---|---|
| 256 | 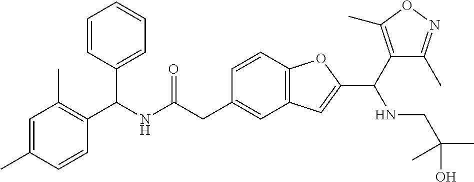 2-(2-((3,5-dimethylisoxazol-4- yl)((2-hydroxy-2- methylpropyl)amino)methyl) benzofuran-5-yl)-N-((2,4- dimethylphenyl)(phenyl)methyl) acetamide | ¹H NMR (MEOD, 400 MHz): δ 7.49 (s, 1H), 7.36 (d, J = 8.8 Hz, 1H), 7.22-7.29 (m, 4H), 7.14 (d, J = 7.2 Hz, 2H), 6.93-6.98 (m, 3H), 6.69 (s, 1H), 6.28 (s, 1H), 5.01 (s, 1H), 2.50- 2.65 (m, 2H), 2.41 (s, 3H), 2.28 (s, 3H), 2.26 (s, 3H), 2.16 (s, 3H), 1.25 (s, 6H). | LC-MS: 477; Rt = 2.04 min |

### Biological Data

As stated above, the compounds according to Formula (I) are RORγ modulators, and are useful in the treatment of diseases mediated by RORγ. The biological activities of the compounds according to Formula (I) can be determined using any suitable assay for determining the activity of a candidate compound as a RORγ modulator, as well as tissue and in vivo models.

### Dual Fluorescence Energy Transfer (FRET) Assay

This assay is based on the knowledge that nuclear receptors interact with cofactors (transcription factors) in a ligand dependent manner. RORγ is a typical nuclear receptor in that it has an AF2 domain in the ligand binding domain (LBD) which interacts with co-activators. The sites of interaction have been mapped to the LXXLL motifs in the co-activator SRC1(2) sequences. Short peptide sequences containing the LXXLL motif mimic the behavior of full-length co-activator.

The assay measures ligand-mediated interaction of the co-activator peptide with the purified bacterial-expressed RORγ ligand binding domain (RORγ-LBD) to indirectly assess ligand binding. RORγ has a basal level of interaction with the co-activator SRC1(2) in the absence of ligand, thus it is possible to find ligands that inhibit or enhance the RORγ/SRC1(2) interaction.

### Materials

### Generation of RORγ-LBD Bacterial Expression Plasmid

Human RORγ Ligand Binding Domain (RORγ-LBD) was expressed in E. coli strain BL21(DE3) as an amino-terminal polyhistidine tagged fusion protein. DNA encoding this recombinant protein was sub-cloned into a modified pET21a expression vector (Novagen). A modified polyhistidine tag (MKKHHHHHHLVPRGS) (SEQ ID No: 1) was fused in frame to residues 263-518 of the human RORγ sequence.

### Protein Purification

Approximately 50 g E. coli cell pellet was resuspended in 300 mL of lysis buffer (30 mM imidazole pH 7.0 and 150 mM NaCl). Cells were lysed by sonication and cell debris was removed by centrifugation for 30 minutes at 20,000 g at 4° C. The cleared supernatant was filtered through a 0.45 μM cellulose acetate membrane filter. The clarified lysate was loaded onto a column (XK-26) packed with ProBond Nickel Chelating resin (InVitrogen), pre-equilibrated with 30 mM imidazole pH 7.0 and 150 mM NaCl. After washing to baseline absorbance with the equilibration buffer, the column was developed with a gradient from 30 to 500 mM imidazole pH 7.0. Column fractions containing the RORγ-LBD protein were pooled and concentrated to a volume of 5 mL. The concentrated protein was loaded onto a Superdex 200 column pre-equilibrated with 20 mM Tris-Cl pH 7.2 and 200 mM NaCl. The fractions containing the desired RORγ-LBD protein were pooled together.

### Protein Biotinylation

Purified RORγ-LBD was buffer exchanged by exhaustive dialysis [3 changes of at least 20 volumes (>8000×)]against PBS [100 mM NaPhosphate, pH 8 and 150 mM NaC1]. The concentration of RORγ-LBD was approximately 30 μM in PBS. Five-fold molar excess of NHS-LC-Biotin (Pierce) was added in a minimal volume of PBS. This solution was incubated with occasional gentle mixing for 60 minutes at ambient room temperature. The modified RORγ-LBD was dialyzed against 2 buffer changes—TBS pH 8.0 containing 5 mM DTT, 2 mM EDTA and 2% sucrose—each at least 20 times of the volume. The modified protein was distributed into aliquots, frozen on dry ice and stored at −80° C. The biotinylated RORγ-LBD was subjected to mass spectrometric analysis to reveal the extent of modification by the biotinylation reagent. In general, approximately 95% of the protein had at least a single site of biotinylation and the overall extent of biotinylation followed a normal distribution of multiple sites ranged from one to five.

A biotinylated peptide corresponding to amino acid 676 to 700 (CPSSHSSLTERHKILHRLLQEGSPS) (SEQ ID No: 2) of the co-activator steroid receptor coactivator SRC1(2) was generated using similar method.

### Assay

Preparation of Europium labeled SRC1(2) peptide: biotinylated SRC1(2) solution was prepared by adding an appropriate amount of biotinylated SRC1(2) from the 100 μM stock solution to a buffer containing 10 mM of freshly added DTT from solid to give a final concentration of 40 nM. An appropriate amount of Europium labeled Streptavidin was then added to the biotinylated SRC1(2) solution in a tube to give a final concentration of 10 nM. The tube was inverted gently and incubated for 15 minutes at room temperature. Twenty-fold excess biotin from the 10 mM stock solution was added and the tube was inverted gently and incubated for 10 minutes at room temperature.

Preparation of APC labeled RORγ-LBD: biotinylated RORγ-LBD solution was prepared by adding an appropriate amount of biotinylated RORγ-LBD from the stock solution to a buffer containing 10 mM of freshly added DTT from solid to give a final concentration of 40 nM. An appropriate amount of APC labeled Streptavidin was then added to the biotinylated RORγ-LBD solution in a tube to give a final concentration of 20 nM. The tube was inverted gently and incubated for 15 minutes at room temperature. Twenty-fold excess biotin from the 10 mM stock solution was then added and the tube was inverted gently and incubated for 10 minutes at room temperature.

Equal volumes of the above-described Europium labeled SRC1(2) peptide and the APC labeled RORγ-LBD were gently mixed together to give 20 nM RORγ-LBD, 10 nM APC-Strepavidin, 20 nM SRC1(2) and 5 nM Europium-Streptavidin. The reaction mixtures were incubated for 5 minutes. Using a Thermo Combi Multidrop 384 stacker unit, 25 μm, of the reaction mixtures per well was added to the 384-well assay plates containing 1 μm, of test compound per well in 100% DMSO. The plates were incubated for 1 hour and then read on ViewLux in Lance mode for EU/APC.

### Results

All exemplified compounds (Examples 1-256) were tested in the dual FRET assay described above and were found to have a pIC₅₀ between 4.8 and 9.

1. A compound according to Formula (I): 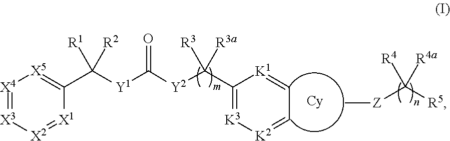
wherein:
m and n are each independently 0, 1, or 2;
X¹, X², X³, X⁴, and X⁵ are each independently selected from N, N⁺—O⁻, CH, and CR⁶, wherein 0-3 of X¹, X², X³, X⁴, and X⁵ are N or N⁺—O⁻ and 0-3 of X¹, X², X³, X⁴, and X⁵ are CR⁶;
one of Y¹ and Y² is O or NR⁸ and the other is a bond;
or X¹ is CR⁶, Y¹ is NR⁸, Y² is a bond, and R⁶ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;
K¹, K², and K³ are each independently selected from N, CH, and CR⁶, wherein 0-2 of K¹, K², and K³ are N and 0-2 of K¹, K², and K³ are CR⁶;
Z is O, C═O, NH, or a bond;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{1a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;
R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;
or R⁴ and R^{4a} are each hydrogen;
or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
R⁵ is phenyl or 5- or 6-membered heteroaryl, wherein said phenyl or heteroaryl is optionally substituted one, two, or three times, independently, by (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)CO₂R⁷, —((C₀-C₃)alkyl)CONR⁷R⁸, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;
R¹⁰ is (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
R¹¹ is (C₁-C₆)alkyl, or (C₁-C₆)haloalkyl; and
Cy taken together with the two carbon atoms of the phenyl or heteroaryl group to which it is fused comprises a five or six membered ring, optionally containing one, two, or three heteroatoms independently selected from oxygen, nitrogen, and sulfur, which ring is optionally substituted one or two times, independently, by R¹⁰;
or a salt thereof.

2. (canceled)

3. The compound or salt according to claim 1, wherein m is 1 and n is 0 or 1.

4. The compound or salt according to claim 1, wherein X¹, X², X³, X⁴, and X⁵ are each independently selected from CH and CR⁶, wherein 0-3 of X¹, X², X³, X⁴, and X⁵ are CR⁶.

5. The compound or salt according to claim 1, wherein X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl((C₁-C₄)alkyl)amino, wherein 2-4 of X², X³, X⁴, and X⁵ are a carbon atom substituted by hydrogen.

6. The compound or salt according to claim 1, wherein Y¹ is NH or NCH₃ and Y² is a bond.

7. The compound or salt according to claim 1, wherein K¹, K², and K³ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 1-3 of K¹, K², and K³ are a carbon atom substituted by hydrogen.

8. The compound or salt according to claim 1, wherein Z is a bond.

9. The compound or salt according to claim 1, wherein R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano (C₁-C₄)alkoxy, or ((C₁-C₄)allyl)((C₁-C₄)allyl)amino.

10. The compound or salt according to claim 1, wherein R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

11. The compound or salt according to claim 1, wherein R² is hydrogen or methyl.

12. The compound or salt according to claim 1, wherein R³ and R^{3a} are each independently hydrogen or methyl.

13. The compound or salt according to claim 1, wherein R⁴ is (C₁-C₄)alkyl, (C₁-C₄)haloalkyl, —SO₂R¹¹, —OR⁹, or —NR⁸R⁹, wherein said (C₁-C₄)alkyl or (C₁-C₄)haloalkyl is optionally substituted by hydroxyl, —OR⁹, —CO₂R⁷, —CONR⁷R⁸, and —NR⁸R⁹.

14. The compound or salt according to claim 1, wherein R⁴ is (C₁-C₄)alkoxy, hydroxy(C₂-C₄)alkoxy, (C₁-C₄)alkoxy(C₂-C₄)alkoxy, amino(C₂-C₄)alko xy, —SO₂(C₁-C₄)allyl, —O((C₁-C₃)allyl)CO₂H, —O((C₁-C₃)alkyl)CO₂(C₁-C₄)allyl, —O((C₁-C₃)alkyl)CONH₂, —O((C₁-C₃)alkyl)CONH(C₁-C₄)allyl, or —O((C₁-C₃)alkyl)CON((C₁-C₄)allyl)((C₁-C₄)allyl).

15. The compound or salt according to claim 1, wherein R^{4a} is hydrogen or methyl.

16. The compound or salt according to claim 1, wherein R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

17. The compound according to claim 1, represented by Formula (Ia): 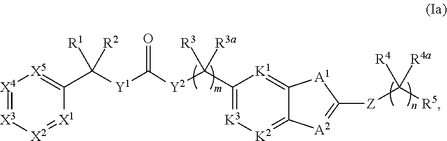
wherein:
m is 1;
n is 0 or 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹ is N, CH, or CR¹⁰;
A² is O, S, NH, or NR⁹;
Z is a bond;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;
or R⁴ and R^{4a} are each hydrogen;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and
R¹¹ is (C₁-C₆)alkyl, or (C₁-C₆)haloalkyl; and
or a pharmaceutically acceptable salt thereof.

18. (canceled)

19. (canceled)

20. The compound according to claim 1, represented by Formula (Ib): 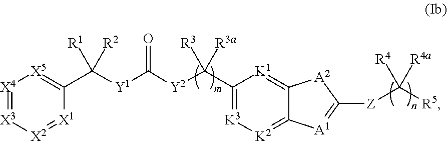
wherein:
m is 1;
n is 0 or 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹ is N, CH, or CR¹⁰;
A² is O, S, NH, or NR⁹;
Z is a bond;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;
or R⁴ and R^{4a} are each hydrogen;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(O—C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and
R¹¹ is (C₁-C₆)alkyl, or (C₁-C₆)haloalkyl;
or a pharmaceutically acceptable salt thereof.

21. (canceled)

22. (canceled)

23. The compound according to claim 1, represented by Formula (Ic): 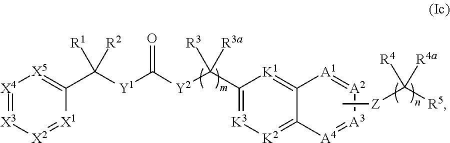
wherein:
m is 1;
n is 0 or 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹, A², A³, and A⁴ are each independently selected from N, C, CH, and CR¹⁰, wherein 0-2 of A¹, A², A³, and A⁴ are N, 0-1 of A¹, A², A³, and A⁴ are CR¹⁰, and 1 of A¹, A², A³, and A⁴ is C to which Z is attached,
Z is a bond;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;
or R⁴ and R^{4a} are each hydrogen;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and
R¹¹ is (C₁-C₆)alkyl, or (C₁-C₆)haloalkyl;
or a pharmaceutically acceptable salt thereof.

24. (canceled)

25. The compound according to claim 1, represented by Formula (Id): 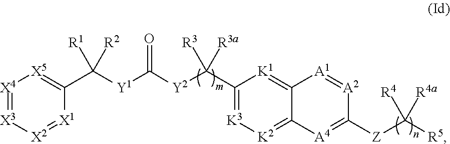
wherein:
mist;
n is 0 or 1;
X¹, X², X³, X⁴, and X⁵ are each independently selected from N, N⁺—O⁻, CH, and CR⁶, wherein 0-2 of X¹, X², X³, X⁴, and X⁵ are N or N⁺—O⁻ and 0-3 of X¹, X², X³, X⁴, and X⁵ are CR⁶;
Y¹ is NH or NCH₃ and Y² is a bond;
K¹, K², and K³ are each independently selected from N, CH, and CR⁶, wherein 0-1 of K¹, K², and K³ are N and 0-1 of K¹, K², and K³ are CR⁶;
A¹, A², and A⁴ are each independently selected from N, CH, and CR¹⁰, wherein 0-2 of A¹, A², and A⁴ are N, and 0-1 of A¹, A², and A⁴ are CR¹⁰;
Z is a bond;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁶;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁶;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
R⁴ is halogen, (C₁-C₆)alkyl, (C₁-C₆)haloalkyl, —SO₂R¹¹, —CO₂R⁷, —CONR⁷R⁸, —OR⁹, or —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹; and R^{4a} is hydrogen, halogen, hydroxyl, amino, or (C₁-C₆)alkyl;
or R⁴ and R^{4a} are each hydrogen;
R⁵ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridinyl N-oxide, pyridazinyl, pyrazinyl, pyrimidinyl, or triazinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;
each R⁶ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by hydroxyl, (C₁-C₄)alkoxy, —CO₂R⁷, —CONH₂, —CONH(C₁-C₄)alkyl, —CON((C₁-C₄)alkyl)((C₁-C₄)alkyl), amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered unsaturated, partially unsaturated or aromatic ring, optionally containing one additional heteroatom selected from oxygen, nitrogen, and sulfur and further optionally containing one or two additional nitrogen atoms, which ring is optionally substituted by one or two substituents independently selected from the group consisting of cyano, (C₁-C₄)alkyl, (C₁-C₄)haloalkyl, (C₃-C₆)cycloalkyl, —CO₂H, —CO₂(C₁-C₄)alkyl, —SO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, and —N((C₁-C₄)alkyl)C(O)R⁷;
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, ((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl; and
R¹¹ is (C₁-C₆)alkyl, or (C₁-C₆)haloalkyl;
or a pharmaceutically acceptable salt thereof.

26-29. (canceled)

30. A pharmaceutical composition comprising the compound, or a pharmaceutically acceptable salt thereof, according to claim 1 and a pharmaceutically acceptable excipient.

31-41. (canceled)


SEQUENCE LISTING (2 sequences)

SEQ ID NO: 1 - protein, 15 aa, Human
MKKHHHHHHLVPRGS

SEQ ID NO: 2 - protein, 25 aa, Artificial Sequence
  Biotinylated peptide
CPSSHSSLTERHKILHRLLQEGSPS